(19) United States
(12) Patent Application Publication
(10) Pub. No.: US 2026/0226073 A1
(43) Pub. Date: Aug. 6, 2026

(54) CBL-B INHIBITORS

(71) Applicant: AURIGENE ONCOLOGY LIMITED, Bangalore (IN)

(72) Inventors: Chandregowda VENKATESHAPPA, Bangalore (IN); Kalisankar BERA, Bangalore (IN); Susanta SAMAJDAR, Bangalore (IN); Olli TÖRMÄKANGAS, Espoo (FI); Ainoleena TURKU, Espoo (FI)

(21) Appl. No.: 19/151,541

(22) PCT Filed: Jan. 26, 2024
(86) PCT No.: PCT/FI2024/050031

§ 371 Date: Jul. 28, 2025

(30) Foreign Application Priority Data
Jan. 28, 2023 (IN) 202311005650
Aug. 19, 2023 (IN) 202311055644

(51) Int. Cl. C07D 491/107; A61K 31/4196; A61K 31/4439; A61K 31/444; A61K 31/454; A61K 31/4545; A61K 31/4725; A61K 31/496; A61K 31/4985; A61K 31/4995; A61K 31/506; A61K 31/5377; A61K 31/55; C07B 59/00; C07D 401/14; C07D 403/10; C07D 403/14; C07D 405/14; C07D 413/14; C07D 417/14; C07D 471/04; C07D 487/04; C07D 487/08; C07D 491/048

(52) U.S. Cl. CPC C07D 491/107; A61K 31/4196; A61K 31/4439; A61K 31/444; A61K 31/454; A61K 31/4545; A61K 31/4725; A61K 31/496; A61K 31/4985; A61K 31/4995; A61K 31/506; A61K 31/5377; A61K 31/55; C07B 59/002; C07D 401/14; C07D 403/10; C07D 403/14; C07D 405/14; C07D 413/14; C07D 417/14; C07D 471/04; C07D 487/04; C07D 487/08; C07D 491/048; C07B 2200/05

(57) ABSTRACT

The present invention relates to a compound of formula (I) wherein Z, M, R₁, R₂ and R₃ are as defined in claim 1, or a pharmaceutically acceptable salt thereof. The compounds of formula (I) possess utility as inhibitors of Cbl-b. The compounds are useful as medicaments in the treatment of diseases or conditions wherein inhibition of CBl-b is desired, such as cancer.

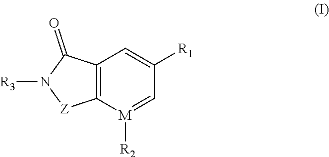

### TECHNICAL FIELD

The present invention relates to therapeutically active compounds useful in the inhibition of E3 ligase Casitas B-lineage lymphoma b enzyme also known as Cbl-b, and to pharmaceutical compositions containing such compounds. The compounds are useful, for example, for modulating immune system in the treatment of various diseases and disorders, such as cancer.

### BACKGROUND OF THE INVENTION

Maintenance of immunological tolerance and induction of T-cell anergy is critical for prevention of autoimmunity. However, in the case of malignancies, tumor-induced T-cell anergy leads to cancer-associated immune paralysis, resulting in uncontrolled tumor growth and metastasis. Proteins that negatively regulate the amplitude and duration of TCR signalling would play critical roles in determining the induction of T cell anergy. Cbl family of E3 ubiquitin ligases, which includes Cbl-b and c-Cbl, have emerged as key proteins that modulate several signalling pathways in T cells, NK cells, and B cells by ubiquitination of several activating tyrosine kinases (Bachmaier, K. et al., Nature, 403, 6766, 211-216, 2000).

Ubiquitination is an important post-transcriptional mechanism that regulates cellular function by proteasomal degradation of proteins involved in several signalling pathways. Ubiquitination of target proteins occurs stepwise with three enzymes. Ubiquitination is initiated by catalyses of ubiquitin activation by first enzyme (E1). The activated ubiquitin is then transferred from E1 to Ubiquitin conjugating enzymes (E2). Finally, the third enzyme (E3 ligases) confers substrate specificity and catatalyse the transfer of ubiquitin from E2 into the target protein substrate. Addition of poly-ubiquitin chains to protein marks it for degradation by the proteasomal degradation machinery (Staub, O. et al., Physiological Reviews, 86, 2, 669-707, 2006).

Ubiquitination regulates multiple biological functions including cell division, DNA repair and cellular signalling. Molecules that can modulate components of the ubiquitin proteosome system can be used as therapeutic agents for a wide range of conditions, including cancer and autoimmune diseases.

E3 ubiquitin ligases confer specificity and their role in fine tuning of immunological responses have been described recently. Casitas B-lineage lymphoma proto-oncogene-b (Cbl-b) stands out as one of the most important gate keepers of immune activation due to its function as a non-redundant negative regulator of immune activation (Chiang, Y. et al., Nature, 403, 6766, 16-220, 2000).

Cbl family of E3 ligases selectively regulate activated PTKs by recognizing specific phospho tyrosine containing motifs. Cbl-b also downregulates signalling from antigen and cytokine receptors through ubiquitination of receptor chains and associated cytosolic tyrosine kinases, leading to inactivation and/or proteasomal degradation of target proteins (Schmidt. M. H. et al., Nat Rev Mol Cell Biol, 2005, 6(12):907-918).

Cbl-b is preferentially expressed in peripheral lymphoid organs and acts as a major regulator of immune cell activation and maintains peripheral tolerance. Cbl-b deficient immune cells display lower activation thresholds and Cbl-b knockout mice rejects tumor mediated by cytotoxic T cells and NK cells (Loeser, S. et al., Journal of Experimental Medicine, 204, 4, 879-891, 2007).

Cbl proteins not only regulate adaptive immune cell functions but are also critically involved in the regulation of innate lymphocyte populations, such as NK cells, dendritic cells and myeloid cells. Thus, targeting Cbl-b provides opportunities to enhance anti-cancer immunity.

Compounds with Cbl-b inhibition activity have been disclosed, for example, in WO 2019/148005, WO 2020/210508, WO 2020/236654, WO 2020/264398, WO 2022/169997, WO 2022/217276 and WO 2022/272248.

### SUMMARY OF THE INVENTION

It has been found that compounds of formula (I) are potent inhibitors of E3 ligase Casitas B-lineage lymphoma b enzyme (Cbl-b). The compounds are therefore useful for the treatment of conditions and diseases where inhibition of Cbl-b is desired. Such conditions and diseases include, but are not limited to, cancer.

The present invention relates to a compound of formula (I) or a pharmaceutically acceptable salt thereof

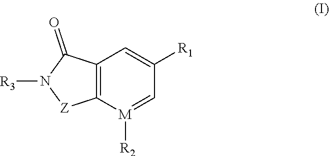

wherein
Z is CH₂ or NH;
M is C or N;
when M is C, then R₂ is hydrogen, halogen, C₁₋₇ alkoxy, cyano, halogen C₁₋₇ alkyl or —NH—C₁₋₇ alkyl;
when M is N, then R₂ is absent;
R₁ is any of the following groups

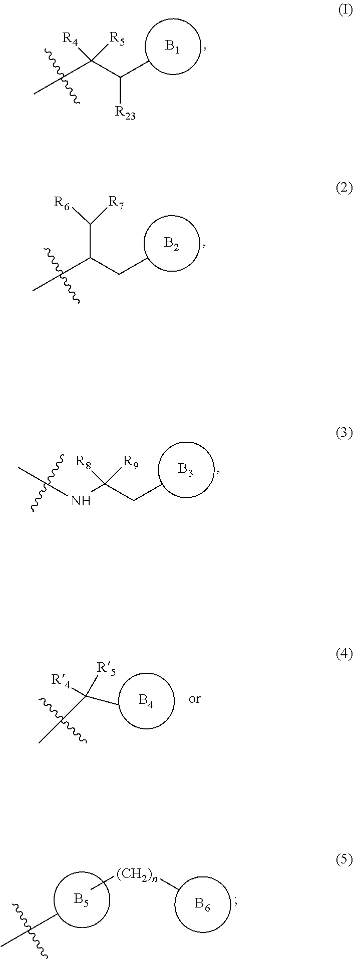

R₄, R₅, R₆, R₇, R₈ and R₉, are, independently, hydrogen, C₁₋₇ alkyl, halogen or halogen C₁₋₇ alkyl, or R₄ and R₅, or R₆ and R₇, or R₈ and R₉ together with the carbon atom to which they are attached form an optionally substituted C₃₋₇ cycloalkyl ring or an optionally substituted 3-6 membered heterocyclic ring having 1-3 heteroatoms as ring atoms independently selected from O, S and N;
R′₄ and R′₅ together with the carbon atom to which they are attached form an optionally substituted C₃-C₆ cycloalkyl ring or an optionally substituted 3-6 membered heterocyclic ring having 1-3 heteroatoms as ring atoms independently selected from O, S and N;
B₁, B₂, B₃, and B₄ are, independently, an optionally substituted 5-6 membered heterocyclic ring having 1-4 heteroatoms as ring atoms independently selected from O, S and N;
B₅ is an optionally substituted phenyl ring or an optionally substituted 5-6 membered heterocyclic ring having 1-4 heteroatoms as ring atoms independently selected from O, S and N;
B₆ is an optionally substituted 5-10 membered monocyclic, bicyclic or spiro bicyclic heterocyclic ring containing 1-4 heteroatoms as ring atoms independently selected from O, S and N;
R₃ is any of the following groups

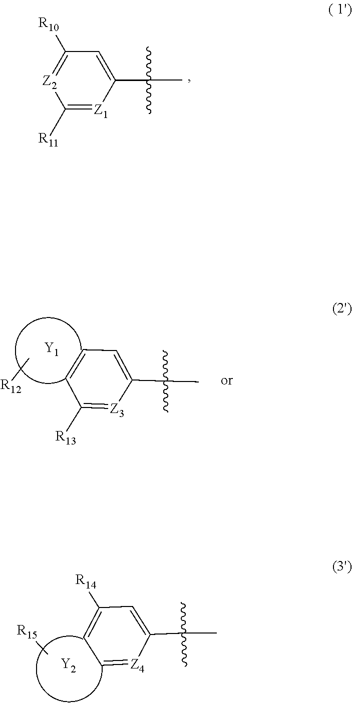

Z₁, Z₂, Z₃ and Z₄ are, independently, CH, CX, C—CF₃ or N;
X is a halogen;
Y₁ and Y₂ are, independently, a 3-6 membered carbocyclic ring or a 5-6 membered heterocyclic ring having 1-4 heteroatoms as ring atoms independently selected from O, S and N;
R₁₁, R₁₃ and R₁₅ are, independently, hydrogen, C₁₋₇ alkyl, C₂₋₇ alkenyl, C₃₋₇ cycloalkyl, C₃₋₇ cycloalkyl C₁₋₇ alkyl, halogen, C₁₋₇ alkoxy, halogen C₁₋₇ alkyl, or a 3-6 membered heterocyclic ring having 1-3 heteroatoms as ring atoms independently selected from O, S and N;
R₁₂ is hydrogen, C₁₋₇ alkyl or —C₁₋₇ alkyl-NR₁₆R₁₇;
R₁₀ and R₁₄, are, independently, —C(O)O—C₁₋₇ alkyl, hydroxy C₁₋₇ alkyl, halogen C₁₋₇ alkyl, —NR₂₁R₂₂, —C₁₋₇ alkyl-NR₂₁R₂₂, -hydroxy C₁₋₇ alkyl-NR₂₁R₂₂, or a group

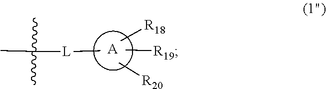

L is a bond, —CH₂—, —CH—, —CH(CH₃)—, —C(CH₂)— or —C(O)—;
A is a 5-10 membered monocyclic, bicyclic or spiro bicyclic heterocyclic ring containing 1-4 heteroatoms as ring atoms independently selected from O, S and N;
R₁₆ and R₁₇ are, independently, hydrogen or C₁₋₇ alkyl;
R₁₈, R₁₉ and R₂₀ are, independently, absent, hydrogen, C₁₋₇ alkyl, C₁₋₇ alkoxy, halogen, cyano, halogen C₁₋₇ alkyl, hydroxy, hydroxy C₁₋₇ alkyl or oxo;
R₂₁ and R₂₂ are, independently, hydrogen, C₁₋₇ alkyl, C₃₋₇ cycloalkyl, C₃₋₇ cycloalkyl C₁₋₇ alkyl, C₁₋₇ alkyl C₃₋₇ cycloalkyl, (C₃₋₇ cycloalkyl)₂ C₁₋₇ alkyl, C₁₋₇ alkyl C₃₋₇ cycloalkyl C₁₋₇ alkyl, C₃₋₇ cycloalkyl halogen C₁₋₇ alkyl, halogen C₁₋₇ alkyl C₃₋₇ cycloalkyl C₁₋₇ alkyl, halogen C₁₋₇ alkyl, halogen C₃₋₇ cycloalkyl, halogen C₃₋₇ cycloalkyl C₁₋₇ alkyl, hydroxy C₁₋₇ alkyl, hydroxy C₁₋₇ alkyl C₃₋₇ cycloalkyl C₁₋₇ alkyl, hydroxy C₃₋₇ cycloalkyl, C₁₋₇ alkoxy C₃₋₇ cycloalkyl, cyano C₃₋₇ cycloalkyl C₁₋₇ alkyl, hydroxy C₃₋₇ cycloalkyl C₁₋₇ alkyl, C₁₋₇ alkoxy C₃₋₇ cycloalkyl C₁₋₇ alkyl, C₁₋₇ alkoxy C₁₋₇ alkyl, aminohydroxy C₁₋₇ alkyl, C₁₋₇ alkyl amide C₁₋₇ alkyl, —SO₂—C₁₋₇ alkyl, —S(O)—C₁₋₇ alkyl, —C₁₋₇ alkyl-(O—C₁₋₇ alkyl)₁₋₃-NH₂, —C₁₋₇ alkyl (C₃₋₇ cycloalkyl)(OH), or a group

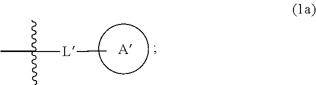

L′ is a bond, C₁₋₇ alkyl or C₂₋₇ alkenyl;
A′ is an optionally substituted 3-6 membered heterocyclic ring having 1-3 heteroatoms as ring atoms independently selected from O, S and N;
R₂₃ is independently, hydrogen, C₁₋₇ alkyl or halogen;
n is 0, 1 or 2;
wherein optional substitution, in each occurrence, is 1-2 substituents independently selected from C₁₋₇ alkyl, C₃₋₇ cycloalkyl, halogen, hydroxy, C₁₋₇ alkoxy, halogen C₁₋₇ alkyl and cyano.

According to one embodiment, the invention provides a method for the treatment of a disease or condition wherein inhibition of Cbl-b is desired comprising administering to a subject in need thereof a therapeutically effective amount of a compound of formula (I) or a pharmaceutically acceptable salt thereof

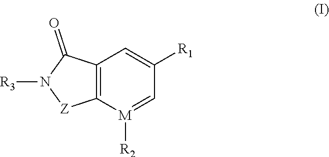

wherein
Z is CH₂ or NH;
M is C or N;
when M is C, then R₂ is hydrogen, halogen, C₁₋₇ alkoxy, cyano, halogen C₁₋₇ alkyl or —NH—C₁₋₇ alkyl;
when M is N, then R₂ is absent;
R₁ is any of the following groups

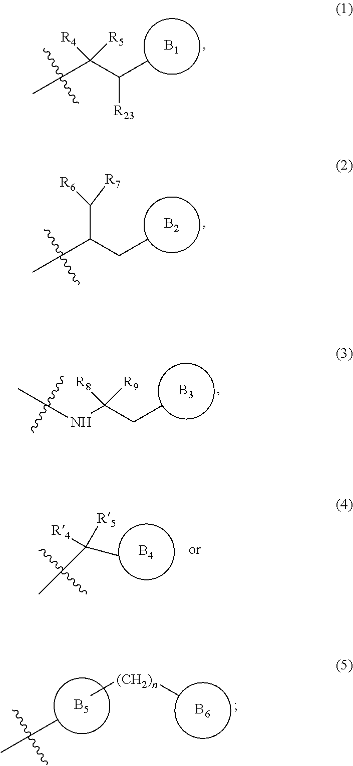

R₄, R₅, R₆, R₇, R₈ and R₉, are, independently, hydrogen, C₁₋₇ alkyl, halogen or halogen C₁₋₇ alkyl, or R₄ and R₅, or R₆ and R₇, or R₈ and R₉ together with the carbon atom to which they are attached form an optionally substituted C₃₋₇ cycloalkyl ring or an optionally substituted 3-6 membered heterocyclic ring having 1-3 heteroatoms as ring atoms independently selected from O, S and N;
R′₄ and R′₅ together with the carbon atom to which they are attached form an optionally substituted C₃-C₆ cycloalkyl ring or an optionally substituted 3-6 membered heterocyclic ring having 1-3 heteroatoms as ring atoms independently selected from O, S and N;
B₁, B₂, B₃, and B₄ are, independently, an optionally substituted 5-6 membered heterocyclic ring having 1-4 heteroatoms as ring atoms independently selected from O, S and N;
B₅ is an optionally substituted phenyl ring or an optionally substituted 5-6 membered heterocyclic ring having 1-4 heteroatoms as ring atoms independently selected from O, S and N;
B₆ is an optionally substituted 5-10 membered monocyclic, bicyclic or spiro bicyclic heterocyclic ring containing 1-4 heteroatoms as ring atoms independently selected from O, S and N;
R₃ is any of the following groups

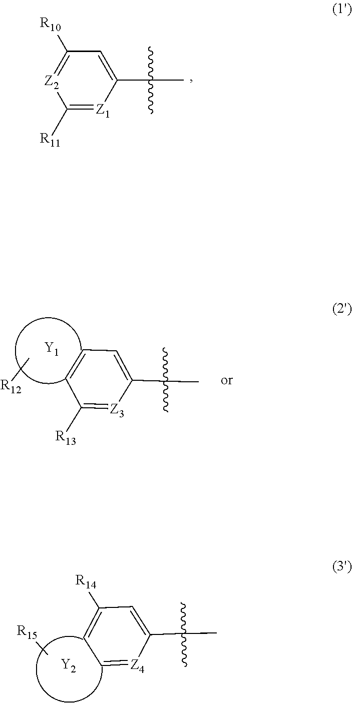

Z₁, Z₂, Z₃ and Z₄ are, independently, CH, CX, C—CF₃ or N;
X is a halogen;
Y₁ and Y₂ are, independently, a 3-6 membered carbocyclic ring or a 5-6 membered heterocyclic ring having 1-4 heteroatoms as ring atoms independently selected from O, S and N;
R₁₁, R₁₃ and R₁₅ are, independently, hydrogen, C₁₋₇ alkyl, C₂₋₇ alkenyl, C₃₋₇ cycloalkyl, C₃₋₇ cycloalkyl C₁₋₇ alkyl, halogen, C₁₋₇ alkoxy, halogen C₁₋₇ alkyl, or a 3-6 membered heterocyclic ring having 1-3 heteroatoms as ring atoms independently selected from O, S and N;
R₁₂ is hydrogen, C₁₋₇ alkyl or —C₁₋₇ alkyl-NR₁₆R₁₇;
R₁₀ and R₁₄, are, independently, —C(O)O—C₁₋₇ alkyl, hydroxy C₁₋₇ alkyl, halogen C₁₋₇ alkyl, —NR₂₁R₂₂, —C₁₋₇ alkyl-NR₂₁R₂₂, -hydroxy C₁₋₇ alkyl-NR₂₁R₂₂, or a group

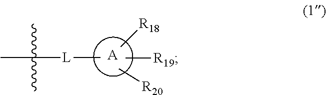

L is a bond, —CH₂—, —CH—, —CH(CH₃)—, —C(CH₂)— or —C(O)—;
A is a 5-10 membered monocyclic, bicyclic or spiro bicyclic heterocyclic ring containing 1-4 heteroatoms as ring atoms independently selected from O, S and N;
R₁₆ and R₁₇ are, independently, hydrogen or C₁₋₇ alkyl;
R₁₈, R₁₉ and R₂₀ are, independently, absent, hydrogen, C₁₋₇ alkyl, C₁₋₇ alkoxy, halogen, cyano, halogen C₁₋₇ alkyl, hydroxy, hydroxy C₁₋₇ alkyl or oxo;
R₂₁ and R₂₂ are, independently, hydrogen, C₁₋₇ alkyl, C₃₋₇ cycloalkyl, C₃₋₇ cycloalkyl C₁₋₇ alkyl, C₁₋₇ alkyl C₃₋₇ cycloalkyl, (C₃₋₇ cycloalkyl)₂ C₁₋₇ alkyl, C₁₋₇ alkyl C₃₋₇ cycloalkyl C₁₋₇ alkyl, C₃₋₇ cycloalkyl halogen C₁₋₇ alkyl, halogen C₁₋₇ alkyl C₃₋₇ cycloalkyl C₁₋₇ alkyl, halogen C₁₋₇ alkyl, halogen C₃₋₇ cycloalkyl, halogen C₃₋₇ cycloalkyl C₁₋₇ alkyl, hydroxy C₁₋₇ alkyl, hydroxy C₁₋₇ alkyl C₃₋₇ cycloalkyl C₁₋₇ alkyl, hydroxy C₃₋₇ cycloalkyl, C₁₋₇ alkoxy C₃₋₇ cycloalkyl, cyano C₃₋₇ cycloalkyl C₁₋₇ alkyl, hydroxy C₃₋₇ cycloalkyl C₁₋₇ alkyl, C₁₋₇ alkoxy C₃₋₇ cycloalkyl C₁₋₇ alkyl, C₁₋₇ alkoxy C₁₋₇ alkyl, aminohydroxy C₁₋₇ alkyl, C₁₋₇ alkyl amide C₁₋₇ alkyl, —SO₂—C₁₋₇ alkyl, —S(O)—C₁₋₇ alkyl, —C₁₋₇ alkyl-(O—C₁₋₇ alkyl)₁₋₃-NH₂, —C₁₋₇ alkyl (C₃₋₇ cycloalkyl)(OH), or a group

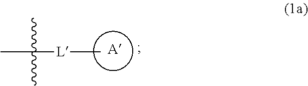

L′ is a bond, C₁₋₇ alkyl or C₂₋₇ alkenyl;
A′ is an optionally substituted 3-6 membered heterocyclic ring having 1-3 heteroatoms as ring atoms independently selected from O, S and N;
R₂₃ is independently, hydrogen, C₁₋₇ alkyl or halogen;
n is 0, 1 or 2;
wherein optional substitution, in each occurrence, is 1-2 substituents independently selected from C₁₋₇ alkyl, C₃₋₇ cycloalkyl, halogen, hydroxy, C₁₋₇ alkoxy, halogen C₁₋₇ alkyl and cyano.

According to one embodiment, the disease or condition wherein inhibition of Cbl-b is desired is cancer, for example, squamous cell carcinoma, bladder cancer, gastric cancer, liver cancer, lung cancer and colon cancer, bone cancer, pancreatic cancer, melanoma, uterine cancer, ovarian cancer, rectal cancer, stomach cancer, testicular cancer, uterine cancer, endometrial cancer, non-Hodgkin's lymphoma or leukemia.

According to one embodiment, the invention provides a pharmaceutical composition comprising a compound of formula (I) or a pharmaceutically acceptable salt thereof together with a pharmaceutically acceptable carrier.

### DETAILED DESCRIPTION OF THE INVENTION

The present application provides novel compounds of formula (I) or pharmaceutically acceptable salts thereof which are useful as Cbl-b inhibitors.

One of the embodiments of the present invention provides a compound of formula (I) or a pharmaceutically acceptable salt thereof

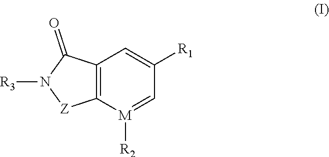

wherein
Z is CH₂ or NH;
M is C or N;
when M is C, then R₂ is hydrogen, halogen, C₁₋₇ alkoxy, cyano, halogen C₁₋₇ alkyl or —NH—C₁₋₇ alkyl;
when M is N, then R₂ is absent;
R₁ is any of the following groups

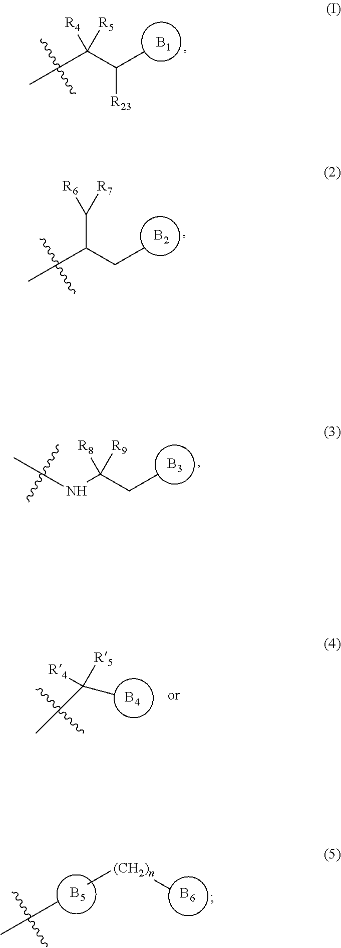

R₄, R₅, R₆, R₇, R₈ and R₉, are, independently, hydrogen, C₁₋₇ alkyl, halogen or halogen C₁₋₇ alkyl, or R₄ and R₅, or R₆ and R₇, or R₈ and R₉ together with the carbon atom to which they are attached form an optionally substituted C₃₋₇ cycloalkyl ring or an optionally substituted 3-6 membered heterocyclic ring having 1-3 heteroatoms as ring atoms independently selected from O, S and N;
R′₄ and R′₅ together with the carbon atom to which they are attached form an optionally substituted C₃-C₆ cycloalkyl ring or an optionally substituted 3-6 membered heterocyclic ring having 1-3 heteroatoms as ring atoms independently selected from O, S and N;
B₁, B₂, B₃, and B₄ are, independently, an optionally substituted 5-6 membered heterocyclic ring having 1-4 heteroatoms as ring atoms independently selected from O, S and N;
B₅ is an optionally substituted phenyl ring or an optionally substituted 5-6 membered heterocyclic ring having 1-4 heteroatoms as ring atoms independently selected from O, S and N;
B₆ is an optionally substituted 5-10 membered monocyclic, bicyclic or spiro bicyclic heterocyclic ring containing 1-4 heteroatoms as ring atoms independently selected from O, S and N;
R₃ is any of the following groups

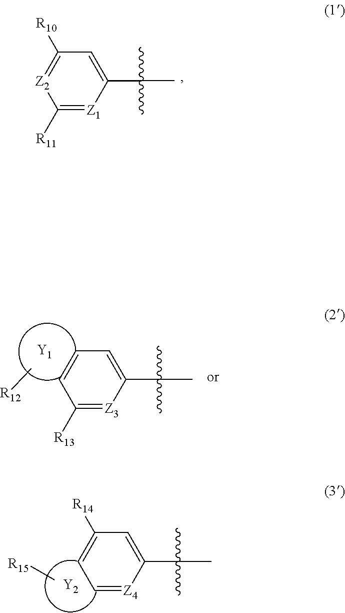

Z₁, Z₂, Z₃ and Z₄ are, independently, CH, CX, C—CF₃ or N;
X is a halogen;
Y₁ and Y₂ are, independently, a 3-6 membered carbocyclic ring or a 5-6 membered heterocyclic ring having 1-4 heteroatoms as ring atoms independently selected from O, S and N;
R₁₁, R₁₃ and R₁₅ are, independently, hydrogen, C₁₋₇ alkyl, C₂₋₇ alkenyl, C₃₋₇ cycloalkyl, C₃₋₇ cycloalkyl C₁₋₇ alkyl, halogen, C₁₋₇ alkoxy, halogen C₁₋₇ alkyl, or a 3-6 membered heterocyclic ring having 1-3 heteroatoms as ring atoms independently selected from O, S and N;
R₁₂ is hydrogen, C₁₋₇ alkyl or —C₁₋₇ alkyl-NR₁₆R₁₇;
R₁₀ and R₁₄, are, independently, —C(O)O—C₁₋₇ alkyl, hydroxy C₁₋₇ alkyl, halogen C₁₋₇ alkyl, —NR₂₁R₂₂, —C₁₋₇ alkyl-NR₂₁R₂₂, -hydroxy C₁₋₇ alkyl-NR₂₁R₂₂, or a group

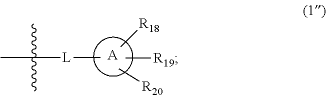

L is a bond, —CH₂—, —CH—, —CH(CH₃)—, —C(CH₂)— or —C(O)—;
A is a 5-10 membered monocyclic, bicyclic or spiro bicyclic heterocyclic ring containing 1-4 heteroatoms as ring atoms independently selected from O, S and N;
R₁₆ and R₁₇ are, independently, hydrogen or C₁₋₇ alkyl;
R₁₈, R₁₉ and R₂₀ are, independently, absent, hydrogen, C₁₋₇ alkyl, C₁₋₇ alkoxy, halogen, cyano, halogen C₁₋₇ alkyl, hydroxy, hydroxy C₁₋₇ alkyl or oxo;
R₂₁ and R₂₂ are, independently, hydrogen, C₁₋₇ alkyl, C₃₋₇ cycloalkyl, C₃₋₇ cycloalkyl C₁₋₇ alkyl, C₁₋₇ alkyl C₃₋₇ cycloalkyl, (C₃₋₇ cycloalkyl)₂ C₁₋₇ alkyl, C₁₋₇ alkyl C₃₋₇ cycloalkyl C₁₋₇ alkyl, C₃₋₇ cycloalkyl halogen C₁₋₇ alkyl, halogen C₁₋₇ alkyl C₃₋₇ cycloalkyl C₁₋₇ alkyl, halogen C₁₋₇ alkyl, halogen C₃₋₇ cycloalkyl, halogen C₃₋₇ cycloalkyl C₁₋₇ alkyl, hydroxy C₁₋₇ alkyl, hydroxy C₁₋₇ alkyl C₃₋₇ cycloalkyl C₁₋₇ alkyl, hydroxy C₃₋₇ cycloalkyl, C₁₋₇ alkoxy C₃₋₇ cycloalkyl, cyano C₃₋₇ cycloalkyl C₁₋₇ alkyl, hydroxy C₃₋₇ cycloalkyl C₁₋₇ alkyl, C₁₋₇ alkoxy C₃₋₇ cycloalkyl C₁₋₇ alkyl, C₁₋₇ alkoxy C₁₋₇ alkyl, aminohydroxy C₁₋₇ alkyl, C₁₋₇ alkyl amide C₁₋₇ alkyl, —SO₂—C₁₋₇ alkyl, —S(O)—C₁₋₇ alkyl, —C₁₋₇ alkyl-(O—C₁₋₇ alkyl)₁₋₃-NH₂, —C₁₋₇ alkyl (C₃₋₇ cycloalkyl)(OH), or a group

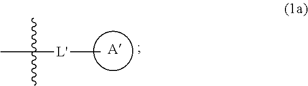

L′ is a bond, C₁₋₇ alkyl or C₂₋₇ alkenyl;
A′ is an optionally substituted 3-6 membered heterocyclic ring having 1-3 heteroatoms as ring atoms independently selected from O, S and N;
R₂₃ is independently, hydrogen, C₁₋₇ alkyl or halogen;
n is 0, 1 or 2;
wherein optional substitution, in each occurrence, is 1-2 substituents independently selected from C₁₋₇ alkyl, C₃₋₇ cycloalkyl, halogen, hydroxy, C₁₋₇ alkoxy, halogen C₁₋₇ alkyl and cyano.

According to one embodiment, specifically provided are compounds according to formula (I) wherein R₁ is group (1) or (5):

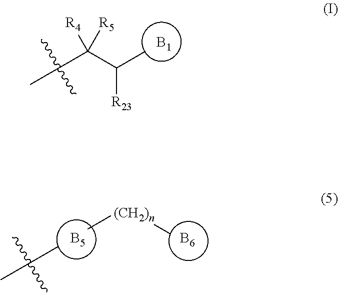

According to yet another embodiment, specifically provided are compounds wherein Z is CH₂. According to yet another embodiment, specifically provided are compounds wherein M is C. According to yet another embodiment, specifically provided are compounds wherein R₂ is hydrogen.

According to yet one embodiment, specifically provided are compounds according to any of the above embodiments wherein R₄ and R₅ are, independently, hydrogen or C₁₋₇ alkyl. According to yet one embodiment, specifically provided are compounds according to any of the above embodiments wherein R₄ is hydrogen and R₅ is or C₁₋₇ alkyl.

According to yet another embodiment, R₄ and R₅ together with the carbon atom to which they are attached form an optionally substituted C₃-C₆ cycloalkyl ring or an optionally substituted 3-6 membered heterocyclic ring having 1-3 heteroatoms as ring atoms independently selected from O, S and N. In a subgroup of the preceding embodiment are compounds, wherein R₄ and R₅ together with the carbon atom to which they are attached form an optionally substituted oxetanyl ring or optionally substituted C₃-C₆ cycloalkyl ring such as optionally substituted cyclobutanyl ring.

According to yet one embodiment, specifically provided are compounds according to any of the above embodiments wherein B₆ is an optionally substituted 5-6 membered heterocyclic ring having 1-4 heteroatoms as ring atoms independently selected from O, S and N.

According to yet one embodiment, specifically provided are compounds according to any of the above embodiments wherein B₁, B₂, B₃, B₄ and B₆ are, independently, a triazolyl ring optionally substituted with one C₁₋₇ alkyl substituent. In a subgroup of the preceding embodiment, the C₁₋₇ alkyl substituent is methyl.

According to yet one embodiment, specifically provided are compounds according to any of the above embodiments wherein R₂₃ is hydrogen.

According to yet one embodiment, specifically provided are compounds according to any of the above embodiments, wherein B₅ is an optionally substituted phenyl ring. According to yet one embodiment, specifically provided are compounds according to any of the above embodiments wherein B₆ is a triazolyl ring optionally substituted with one C₁₋₇ alkyl substituent.

Specifically provided are compounds according to any of the above embodiments wherein n is 0.

According to yet one embodiment, specifically provided are compounds according to any of the above embodiments wherein R₃ is any of the following groups:

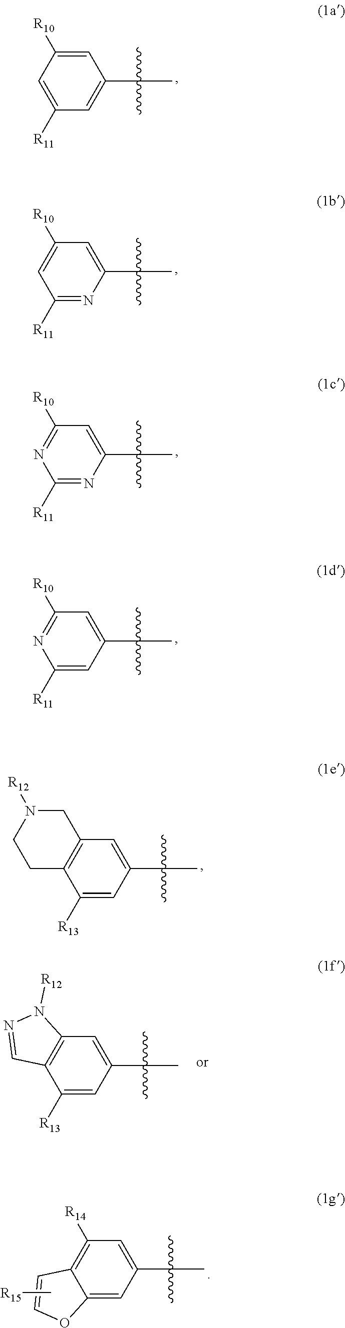

In a subgroup of the preceding embodiment are compounds wherein R₃ is group (1a′), (1b′) or (1c′). In a subgroup of the preceding embodiment are compounds wherein R₃ is group (1b′).

According to yet another embodiment, specifically provided are compounds according to any of the above embodiments wherein R₁₀ and R₁₄ is a group

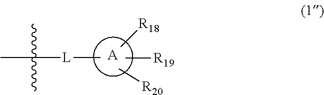

wherein -L- is —CH₂— or —CH(CH₃)—.

In a subgroup of this embodiment are compounds wherein L is —CH₂—.

According to yet another embodiment, specifically provided are compounds according to any of the above embodiments wherein A is a 5-10 membered monocyclic ring containing 1-4 heteroatoms as ring atoms independently selected from O, S and N. In a subgroup of the preceding embodiment, A is any of the following groups:

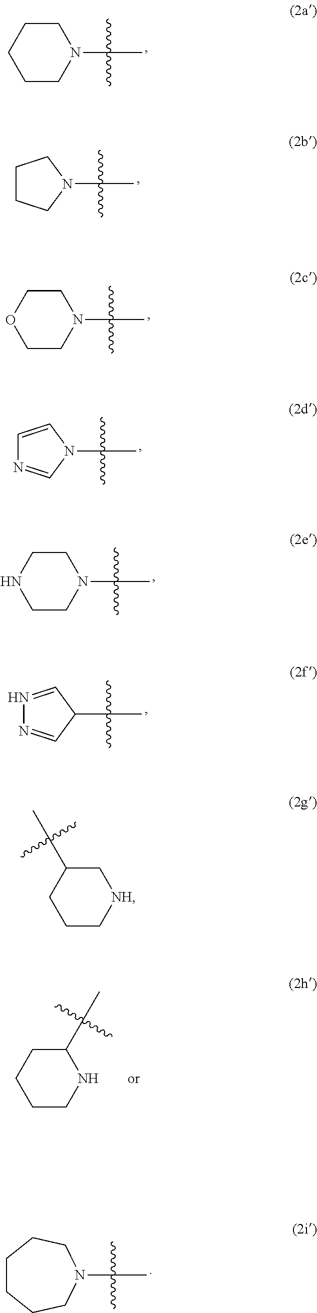

In a subgroup of the preceding embodiment are compounds wherein A is group (2a′), (2b′) or (2l′). In a subgroup of the preceding embodiment are compounds wherein A is group (2a′).

According to yet another embodiment, specifically provided are compounds according to any of the above embodiments wherein A is a 5-10 membered bicyclic ring or spiro bicyclic ring containing 1-4 heteroatoms as ring atoms independently selected from O, S and N. In a subgroup of the preceding embodiment, A is any of the following groups:

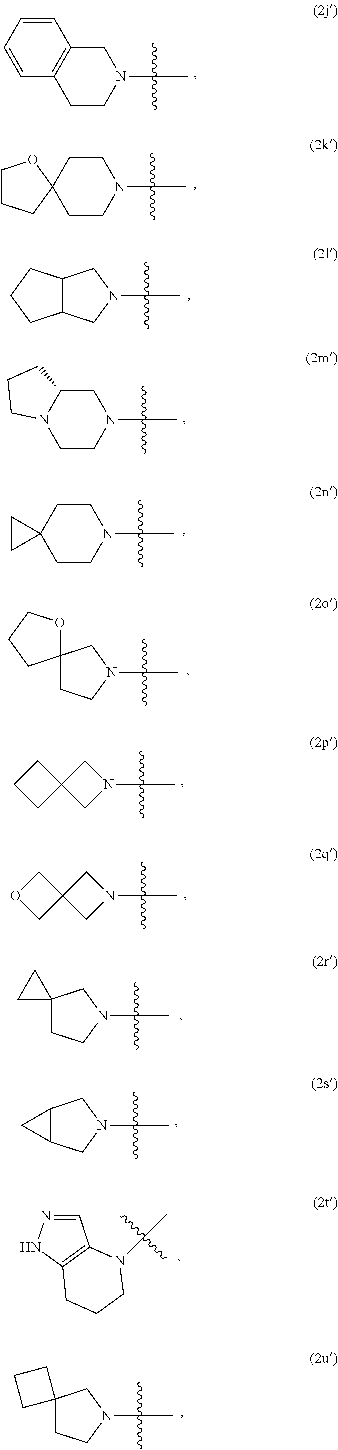

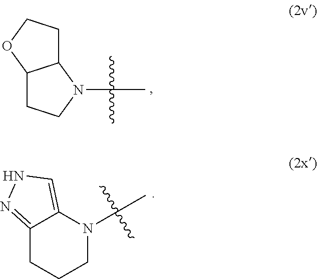 [SMILES: *N1CCC2OCCC21 ; *N1CCCc2n[nH]cc21]

According to one embodiment, specifically provided are compounds according to any of the above embodiments wherein R₁₀ is —C₁₋₇ alkyl-NR₂₁R₂₂. In a subgroup of this embodiment are compounds, wherein —C₁₋₇ alkyl- is —CH₂— or —CH(CH₃)—.

Specifically provided are compounds according to any of the above embodiments wherein R₂₁ is hydrogen.

Specifically provided are compounds according to any of the above embodiments wherein R₂₂ is C₃₋₇ cycloalkyl, C₃₋₇ cycloalkyl C₁₋₇ alkyl, halogen C₁₋₇ alkyl, halogen C₃₋₇ cycloalkyl C₁₋₇ alkyl, hydroxy C₁₋₇ alkyl, C₁₋₇ alkyl C₃₋₇ cycloalkyl C₁₋₇ alkyl, C₁₋₇ alkoxy C₃₋₇ cycloalkyl, hydroxy C₃₋₇ cycloalkyl, hydroxy C₃₋₇ cycloalkyl C₁₋₇ alkyl or a group

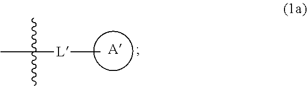

wherein
L′ is a bond or C₁₋₇ alkyl; and
A′ is an optionally substituted 3-6 membered heterocyclic ring having 1-3 heteroatoms as ring atoms independently selected from O, S and N.

In a subgroup of any of the above embodiments are compounds, wherein R₂₁ is hydrogen and R₂₂ is C₃₋₇ cycloalkyl, C₃₋₇ cycloalkyl C₁₋₇ alkyl, halogen C₁₋₇ alkyl, halogen C₃₋₇ cycloalkyl C₁₋₇ alkyl, hydroxy C₁₋₇ alkyl, C₁₋₇ alkyl C₃₋₇ cycloalkyl C₁₋₇ alkyl, C₁₋₇ alkoxy C₃₋₇ cycloalkyl, hydroxy C₃₋₇ cycloalkyl or hydroxy C₃₋₇ cycloalkyl C₁₋₇ alkyl. In a subgroup of this embodiment are compounds, wherein C₃₋₇ cycloalkyl is cyclopropyl, cyclobutyl or cyclopentyl and C₁₋₇ alkyl is C₁₋₃ alkyl, for example —CH₂— or —CH(CH₃)—. In one embodiment provided are compounds wherein halogen C₁₋₇ alkyl is —CH₂—CH₂—CF₃. In one embodiment provided are compounds wherein C₃₋₇ cycloalkyl C₁₋₇ alkyl is —CH₂-cyclopropyl.

Specifically provided are compounds according to any of the above embodiments where compounds are represented by formula (Ia)

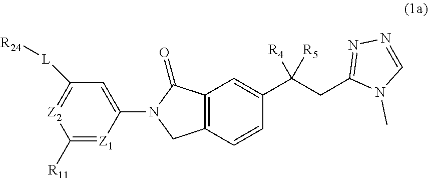

wherein Z₁ and Z₂ are, independently, CH, CX or N;
R₄ and R₅ are, independently hydrogen or C₁₋₇ alkyl, or R₄ and R₅ together with the carbon atom to which they are attached form optionally substituted cyclobutyl ring or an optionally substituted oxetane ring, wherein the optional substitution, in each occurrence, is 1-2 substituents independently selected from C₁₋₇ alkyl, halogen and cyano;
R₁₁ is hydrogen, C₁₋₇ alkyl, C₂₋₇ alkenyl, C₃₋₇ cycloalkyl, C₃₋₇ cycloalkyl C₁₋₇ alkyl, halogen, C₁₋₇ alkoxy or halogen C₁₋₇ alkyl;
L is —CH₂— or —CH(CH₃)—;
R₂₄ is —NR₂₁R₂₂ or a group

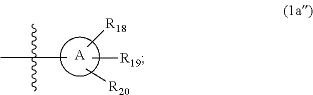

wherein A is a 5-10 membered monocyclic or bicyclic ring or spiro bicyclic heterocyclic ring containing 1-4 heteroatoms as ring atoms independently selected from O, S and N;
R₁₈, R₁₉ and R₂₀ are, independently, hydrogen, C₁₋₇ alkyl, halogen, cyano, halogen C₁₋₇ alkyl, hydroxy, hydroxy C₁₋₇ alkyl or oxo;
R₂₁ and R₂₂ are, independently, hydrogen, C₁₋₇ alkyl, C₃₋₇ cycloalkyl, C₃₋₇ cycloalkyl C₁₋₇ alkyl, C₁₋₇ alkyl C₃₋₇ cycloalkyl, C₁₋₇ alkyl C₃₋₇ cycloalkyl C₁₋₇ alkyl, halogen C₁₋₇ alkyl, halogen C₃₋₇ cycloalkyl or halogen C₃₋₇ cycloalkyl C₁₋₇ alkyl, hydroxy C₁₋₇ alkyl, C₁₋₇ alkoxy C₃₋₇ cycloalkyl, hydroxy C₃₋₇ cycloalkyl C₁₋₇ alkyl or hydroxy C₃₋₇ cycloalkyl.

In a subgroup of the above embodiment are compounds wherein R₄ and R₅ together with the carbon atom to which they are attached form optionally substituted cyclobutyl ring or an optionally substituted oxetane ring, wherein the optional substitution, in each occurrence, is 1-2 substituents independently selected from C₁₋₇ alkyl, halogen and cyano.

In a subgroup of the above embodiment are compounds of formula (Ia) wherein A is any of groups (2a′), (2b′), (2c′), (2d′), (2e′), (2f′), (2g′), (2h′) or (2i′) as defined above. Particularly provided are compounds of formula (Ia), wherein A is group (2a′).

According to yet one embodiment, specifically provided are compounds according to any of the above embodiments wherein R₄ is hydrogen and R₅ is C₁₋₇ alkyl, or R₄ and R₅ together with the carbon atom to which they are attached form a group of formula (3a) or (3b)

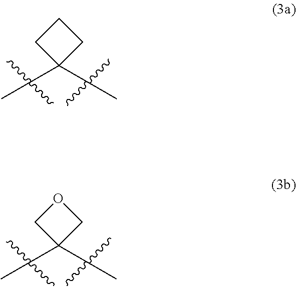 [SMILES: *C1(*)CCC1 ; *C1(*)COC1]

which groups may be optionally substituted with 1-2 substituents independently selected from C₁₋₇ alkyl, halogen and cyano.

According to yet one embodiment, specifically provided are compounds according to any of the above embodiments wherein R₁₈ is hydrogen, and R₁₉ and R₂₀ are, independently, hydrogen, C₁₋₇ alkyl or halogen. In a subgroup of the preceding embodiment are compounds, wherein C₁₋₇ alkyl is methyl. In a subgroup of the preceding embodiment are compounds, wherein R₁₈ and R₁₉ are hydrogen, and R₂₀ is C₁₋₇ alkyl, particularly methyl. In another subgroup are compounds, wherein R₁₈ is hydrogen, and R₁₉ and R₂₀ are C₁₋₇ alkyl, particularly methyl.

Specifically provided are compounds according to any of the above embodiments where compounds are represented by formula (Ib)

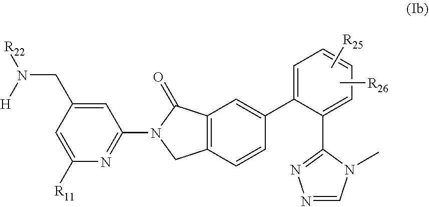

wherein
R₂₅ and R₂₆ are, independently, hydrogen, C₁₋₇ alkyl, C₁₋₇ alkoxy, halogen or cyano;
R₁₁ is hydrogen, C₁₋₇ alkyl or halogen;
R₂₂ is C₁₋₇ alkyl, C₃₋₇ cycloalkyl, C₃₋₇ cycloalkyl C₁₋₇ alkyl, C₁₋₇ alkyl C₃₋₇ cycloalkyl, (C₃₋₇ cycloalkyl)₂ C₁₋₇ alkyl, C₁₋₇ alkyl C₃₋₇ cycloalkyl C₁₋₇ alkyl, C₃₋₇ cycloalkyl halogen C₁₋₇ alkyl, halogen C₁₋₇ alkyl C₃₋₇ cycloalkyl C₁₋₇ alkyl, halogen C₁₋₇ alkyl, halogen C₃₋₇ cycloalkyl, halogen C₃₋₇ cycloalkyl C₁₋₇ alkyl, hydroxy C₁₋₇ alkyl, hydroxy C₁₋₇ alkyl C₃₋₇ cycloalkyl C₁₋₇ alkyl, hydroxy C₃₋₇ cycloalkyl, C₁₋₇ alkoxy C₃₋₇ cycloalkyl, cyano C₃₋₇ cycloalkyl C₁₋₇ alkyl, hydroxy C₃₋₇ cycloalkyl C₁₋₇ alkyl, C₁₋₇ alkoxy C₃₋₇ cycloalkyl C₁₋₇ alkyl, C₁₋₇ alkoxy C₁₋₇ alkyl, aminohydroxy C₁₋₇ alkyl, C₁₋₇ alkyl amide C₁₋₇ alkyl, —SO₂—C₁₋₇ alkyl, —S(O)—C₁₋₇ alkyl, —C₁₋₇ alkyl-(O—C₁₋₇ alkyl)₁₋₃-NH₂, —C₁₋₇ alkyl (C₃₋₇ cycloalkyl)(OH), or a group

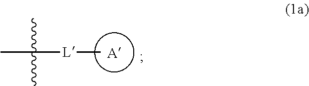

L′ is a bond or C₁₋₇ alkyl;
A′ is an optionally substituted 3-6 membered heterocyclic ring having 1-3 heteroatoms as ring atoms independently selected from O, S and N,
wherein optional substitution, in each occurrence, is 1-2 substituents independently selected from C₁₋₇ alkyl, C₃₋₇ cycloalkyl, halogen, hydroxy, C₁₋₇ alkoxy, halogen C₁₋₇ alkyl and cyano.

In a subgroup R₂₂ is C₃₋₇ cycloalkyl, C₃₋₇ cycloalkyl C₁₋₇ alkyl, halogen C₁₋₇ alkyl, halogen C₃₋₇ cycloalkyl, halogen C₃₋₇ cycloalkyl C₁₋₇ alkyl, C₁₋₇ alkoxy C₃₋₇ cycloalkyl, hydroxy C₃₋₇ cycloalkyl or a group

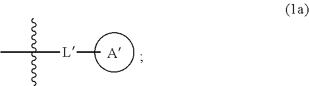

L′ is a bond or C₁₋₇ alkyl;
A′ is an optionally substituted 3-6 membered heterocyclic ring having 1-3 heteroatoms as ring atoms independently selected from O, S and N,
wherein optional substitution, in each occurrence, is 1-2 substituents independently selected from C₁₋₇ alkyl, C₃₋₇ cycloalkyl, halogen, hydroxy, C₁₋₇ alkoxy, halogen C₁₋₇ alkyl and cyano.

In a subgroup of any of the above embodiments are compounds wherein A′ is a furanyl, tetrahydrofuranyl, thiazolyl, pyrazolyl, oxazolyl, oxetanyl, piperidinyl or pyridyl ring.

According to still one embodiment, the present invention provides a method for the treatment of a disease or condition wherein inhibition of Cbl-b is desired, such as cancer, comprising administering to a subject in need thereof a therapeutically effective amount of a compound of formula (I) as defined in any of the above embodiments.

According to one embodiment, the invention provides a method wherein a therapeutically effective amount of a compound of formula (I) is administered in addition to one or more anti-cancer agents.

The compounds of the invention can be prepared by a variety of synthetic routes analogously to the methods known in the literature using suitable starting materials. The compounds according to formula (I) can be prepared e.g. analogously or according to the following reaction Schemes. Some compounds included in the formula (I) can be obtained by converting the functional groups of the other compounds of formula (I) obtained in accordance with the following Schemes, by well known reaction steps such as oxidation, reduction, hydrolysis, acylation, alkylation, amidation, amination, sulfonation and others. It should be noted that any appropriate leaving groups, e.g. N-protecting groups, such as a t-butoxycarbonyl (t-BOC) group or a phenylsulfonyl group, can be used in well known manner during the syntheses in order to improve the selectivity of the reaction steps.

Compounds of formula (I) can be prepared, for example, according to Scheme 1, wherein Z, M, R₁, R₂, R₃ are as defined above and X is a halogen, for example chloro, bromo or iodo. In the method of Scheme 1, the compound of formula [1] is reacted with a halogen compound of formula [2] in a suitable solvent, such as DMF or DMSO, in the presence of a base such as cesium carbonate, and a suitable catalyst system such as a combination Pd(OAc)₂ and 4,5-Bis(diphenylphosphino)-9,9-dimethylxanthene (Xantphos), or copper(I) iodide and potassium iodide, a to produce a compound of formula (I).

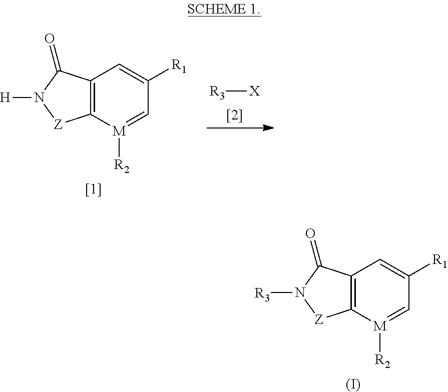

Intermediate compounds can be prepared according to the methods disclosed in the literature or as disclosed in the present disclosure.

For example, intermediate compounds of formula [1], wherein R₁ is a group of formula (1)

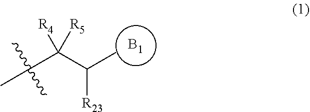

and B₁ is a triazolyl ring optionally substituted with one C₁₋₇ alkyl substituent, can be prepared according to Scheme 2, wherein Z, M, R₂, R₄, R₅ and R₂₃ are as defined above, and X is a halogen, for example chloro or bromo. In the method of Scheme 2, the nitrogen of compound of formula [2a] is first protected with a BOC-group to produce a compound of formula [2b]. The compound of formula [2b] is then reacted with 4,4,5,5-tetramethyl-2-(4,4,5,5-tetramethyl-1,3,2-dioxaborolan-2-yl)-1,3,2-dioxaborolane in a suitable solvent such as 1,4-dioxane in the presence of potassium acetate and [1,1′-bis(diphenylphosphino)ferrocene]dichloropalladium(II) complex. The obtained compound of formula [2c] is then coupled with the compound of formula [2d] in a suitable solvent such as 1,4-dioxane:water mixture in the presence of N,N-diethyl-ethanamine and a suitable catalyst such as [Rh(COD)Cl]₂ to produce a compound of formula [2e]. The protection group is removed and the obtained compound of formula [2f] is treated with hydrazine hydrate. Reacting the compound of formula [2g] with methyl isothiocyanate produces compound of formula [2h] which can be treated with NaOH to produce compound of formula [2i]. Finally, treatment with hydrogen peroxide in a suitable solvent such as DCM in the presence of acetic acid produces the intermediate of formula [1′].

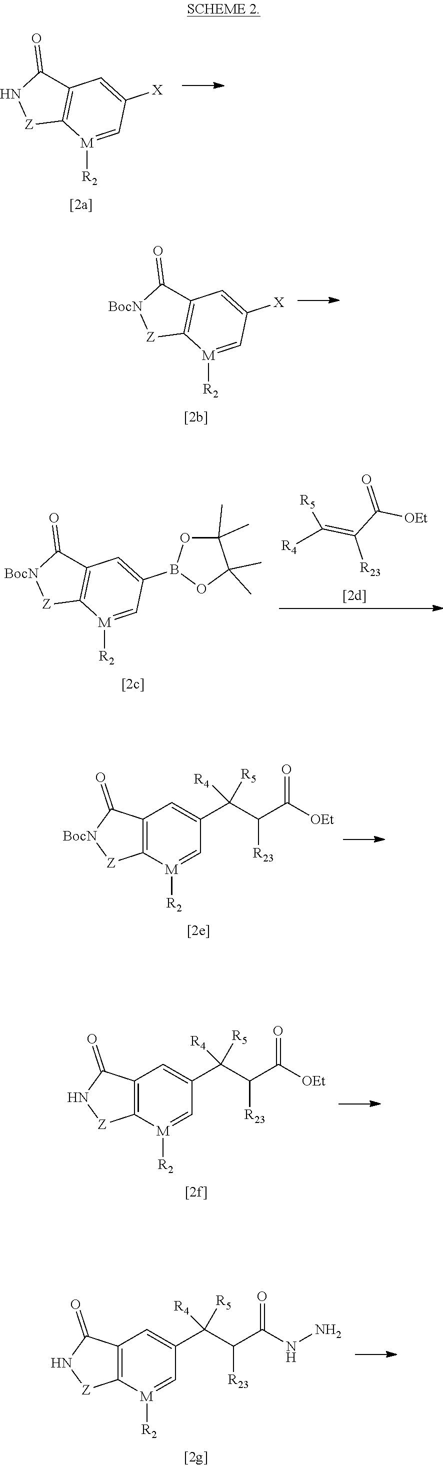

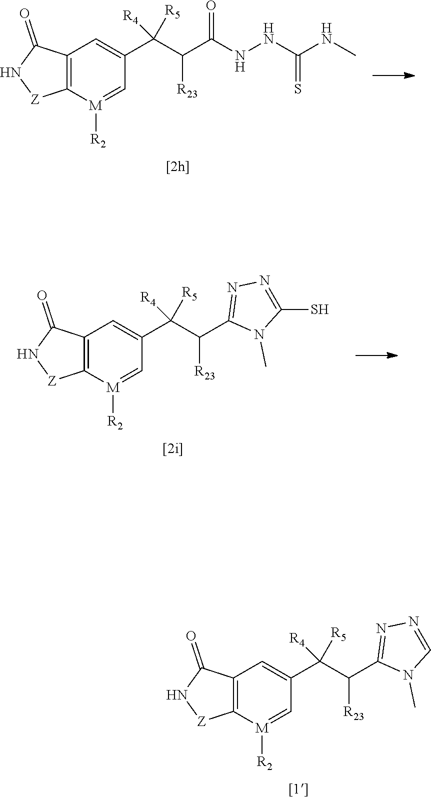

Alternatively, the intermediate of formula [1′] can be prepared according to Scheme 3 wherein Z, M, R₂, R₄, R₅ and R₂₃ are as defined above. In the method of Scheme 3, the compound of formula [3a] is treated with LiOH in a suitable solvent such as a mixture of THF, MeOH and water to produce the compound of formula [3b] which can then be reacted with N-methylhydrazinecarbothioamide in a suitable solvent such as DMF in the presence of DIPEA and HATU to produce the compound of formula [2h]. Following the Scheme 2 the intermediate [1′] is then obtained.

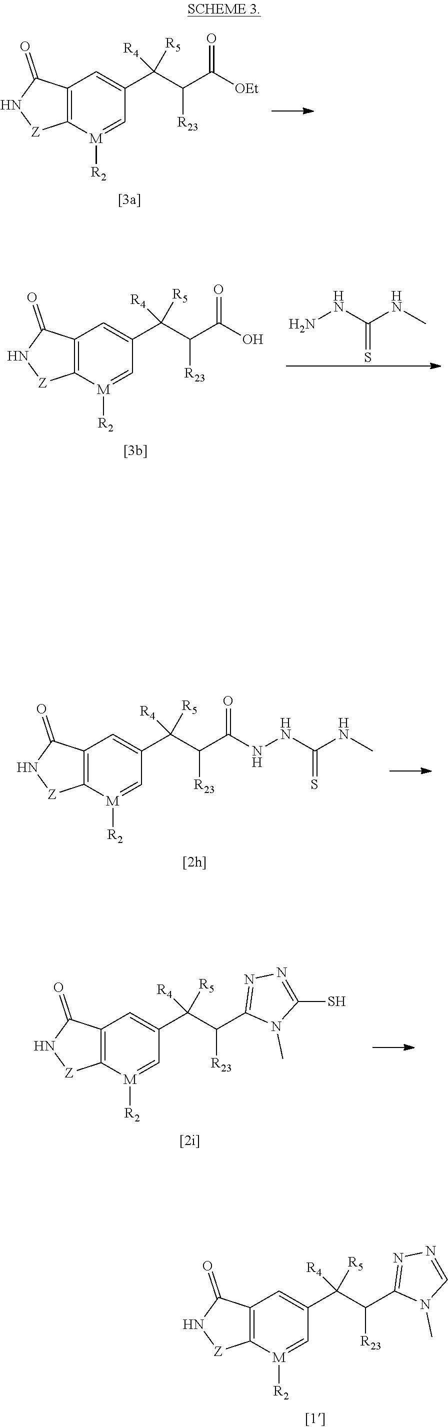

Intermediate compounds R₃—X [2] wherein R₃ is a group of formula (1′) wherein R₁₀ is a group of formula (1″)

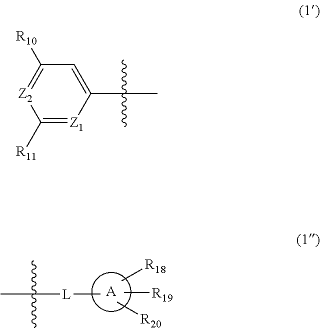

can be prepared, for example, according to Scheme 4, wherein A, Z₁, Z₂, R₁₁, R₁₈, R₁₉ and R₂₀ are as defined above, L is —CH₂— and X is a halogen. In the method of Scheme 4, the cyclic compound of formula [4a] is coupled with the aldehyde compound of formula [4b] in a suitable solvent such as DCM in the presence of DIPEA and sodium tri-acetoxyborohydride (STAB) to produce intermediate [2′].

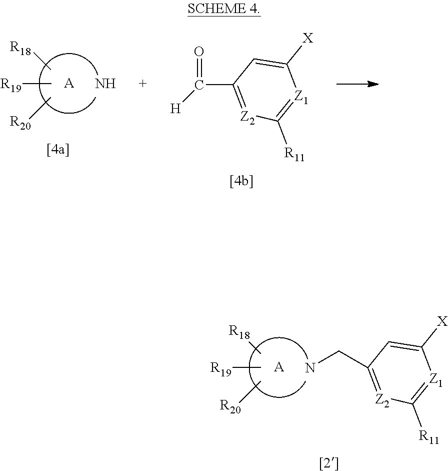

Alternatively, intermediate compounds R₃—X [2] wherein R₃ is a group of formula (1′) wherein R₁₀ is a group of formula (1″)

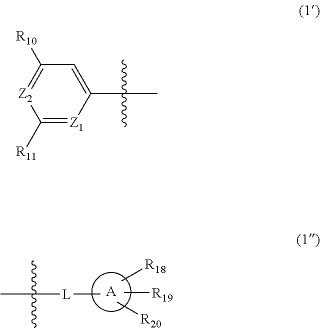

and L is —CH₂— can be prepared according to Scheme 5, wherein A, Z₁, Z₂, R₁₁, R₁₈, R₁₉ and R₂₀ are as defined above, and X is a halogen. In the method of Scheme 5, the aldehyde compound of formula [4b] is reduced with sodium borohydride in a suitable solvent such as THF to produce compound of formula [5a], which is then reacted with methanesulfonyl chloride in a suitable solvent such as DCM in the presence of triethylamine to produce compound of formula [5b]. Coupling the compound of formula [5b] with the compound of formula [4a] in a suitable solvent such as THF in the presence of cesium carbonate produces the intermediate [2′]

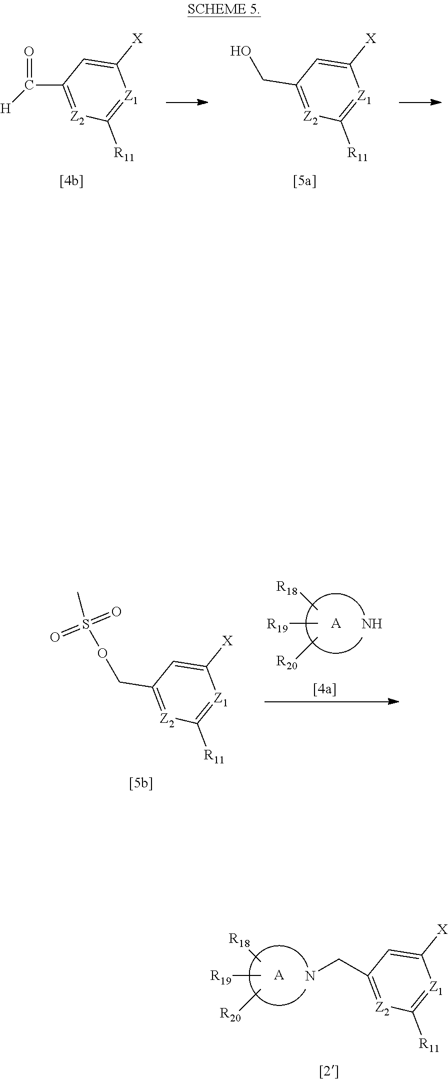

Intermediate compounds R₃—X [2] wherein R₃ is a group of formula (1′) wherein R₁₀ is —C₁₋₇ alkyl-NR₂₁R₂₂

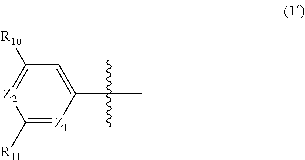

can be prepared, for example, according to Scheme 6, wherein Z₁, Z₂, R₁₁, R₂₁ and R₂₂ are as defined above, and X is a halogen. In the method of Scheme 6, the amine compound of formula [6a] is coupled with the aldehyde compound of formula [4b] in a suitable solvent such as DCM in the presence of acetic acid and sodium triacetoxy-borohydride (STAB) to produce intermediate [3′].

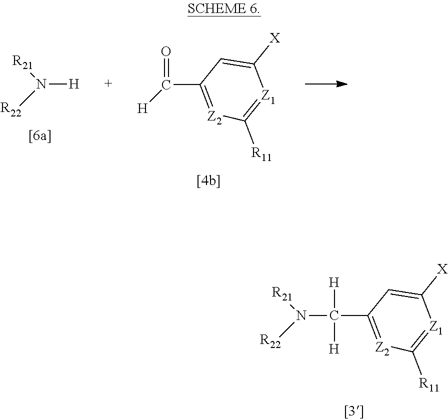

Alternatively, intermediate compounds R₃—X [2] wherein R₃ is a group of formula (1′) wherein R₁₀ is —C₁₋₇ alkyl-NR₂₁R₂₂

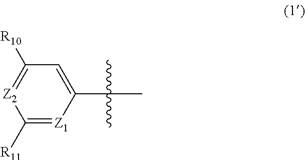

can be prepared, for example, according to Scheme 7, wherein Z₁, Z₂, R₁₁, R₂₁ and R₂₂ are as defined above, and X is a halogen. In the method of Scheme 7, the amine compound of formula [6a] is coupled with carboxylic acid compound of formula [7a] in a suitable solvent such as ACN in the presence of 1-hydroxybenzotriazol (HOBT) and 1-ethyl-3-[3-dimethylaminopropyl]carbodiimide (EDCl) to produce compound of formula [7b], which can be treated with borane dimethylsulfide complex in a suitable solvent such as THF to produce intermediate [4′].

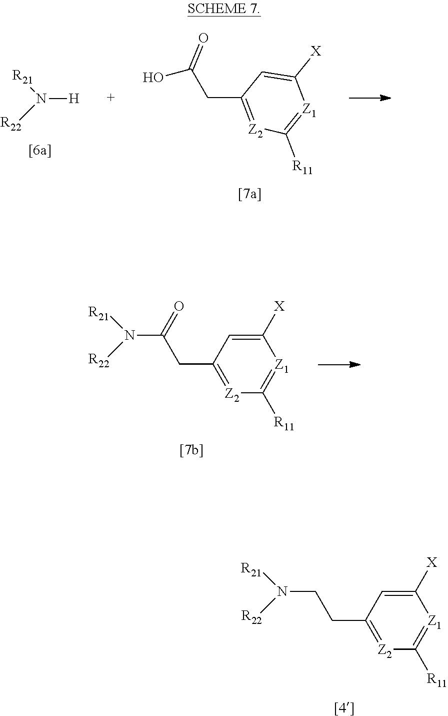

Intermediate compounds R₃—X [2] wherein R₃ is a group of formula (2′)

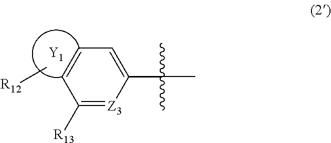

wherein Y₁ a 1H-pyrazolyl ring can be prepared, for example, according to Scheme 8, wherein Z₃ and R₁₃ are as defined above, R₁₂ is hydrogen and X is a halogen. In the method of Scheme 8, the aldehyde compound of formula [8a] is reacted with hydrazine in a suitable solvent such as DMSO in elevated temperature to obtain the intermediate [5′].

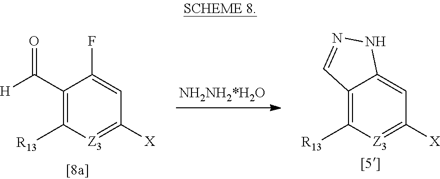

Intermediate compounds R₃—X [2] wherein R₃ is a group of formula (3′) wherein Y₂ is a furanyl ring and R₁₄ is a group of formula (1″) wherein L is —CH₂—

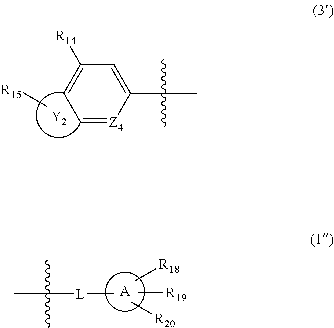

can be prepared, for example, according to Scheme 9, wherein A, Z₄, R₁₈, R₁₉ and R₂₀ are as defined above, L is —CH₂— and X is a halogen. In the method of Scheme 9, the carboxylic acid compound of formula [9a] is treated with H₂SO₄ in MeOH to produce compound of formula [9b], which is reacted with 2-bromo-1,1-diethoxyethane in a suitable solvent such as DMF in the presence of cesium carbonate to produce compound of formula [9c]. Treatment of compound of formula [9c] with polyphosphoric acid in a suitable solvent such as toluene produces compound of formula [9d]. The following reduction with LiBH₄ in a suitable solvent such as THF produces compound of formula [9e], which is then reacted with Dess-Martin periodinane (DMP) in a suitable solvent such as DCM to produce compound of formula [9f]. Coupling of the compound of formula [4a] with the compound of formula [9f] as described in Scheme 4 produces intermediate [6′].

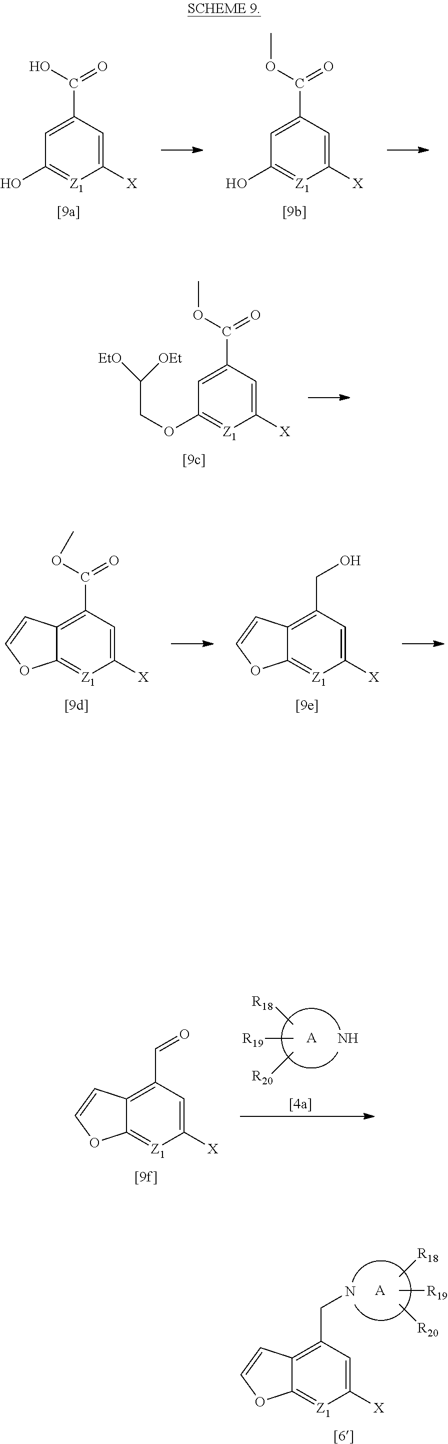

Intermediate compounds R₃—X [2] wherein R₃ is a group of formula (2′)

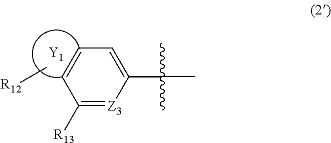

wherein Y₁ a 1,2,3,6-tetrahydropyridinyl ring can be prepared, for example, according to Scheme 10, wherein Z₃, R₁₂ and R₁₃ are as defined above, and X is a halogen. In the method of Scheme 10, the amine compound of formula [10a] is reacted with formaldehyde in a suitable solvent such as acetic acid in the presence of sulfuric acid to produce the intermediate of formula [7′].

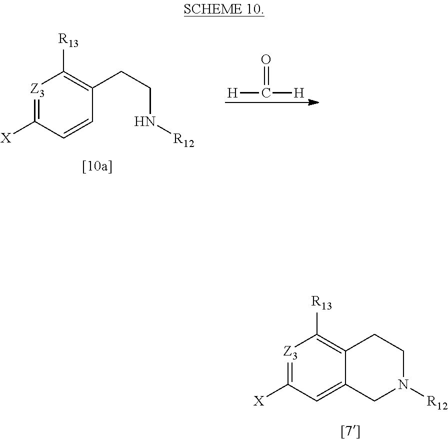

Intermediate compounds R₃—X [2] wherein R₃ is a group of formula (1′) wherein R₁₀ is a group of formula (1″)

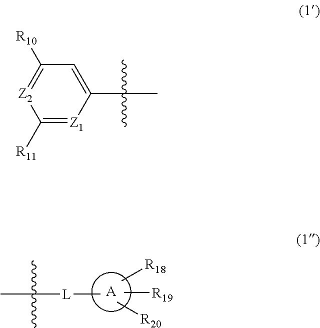

wherein L is —C(O)— can be prepared, for example, according to Scheme 11, wherein A, Z₁, Z₂, R₁₁, R₁₈, R₁₉ and R₂₀ are as defined above, and X is a halogen. In the method of Scheme 11, carboxylic acid compound of formula [11a] is first treated with thionyl chloride in a suitable solvent such as DCM to produce an acid chloride compound of formula [11b] which is then reacted with hydroxyl compound of formula [11c] in a suitable solvent such as DCM in the presence of triethyl amine to produce the intermediate of formula [8′].

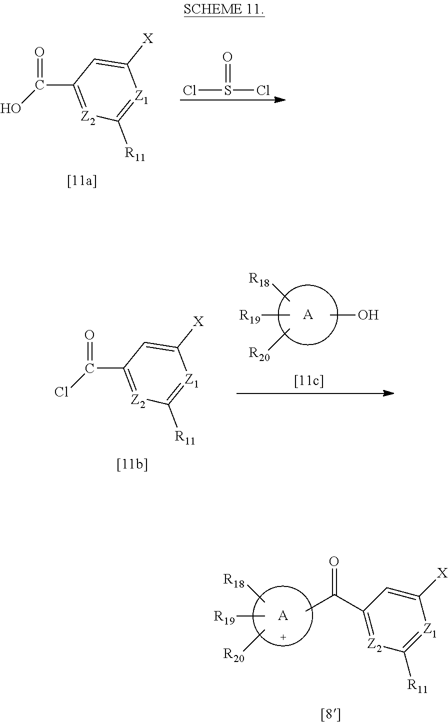

Intermediate compounds R₃—X [2] wherein R₃ is a group of formula (1′) wherein R₁₀ is a group of formula (1″)

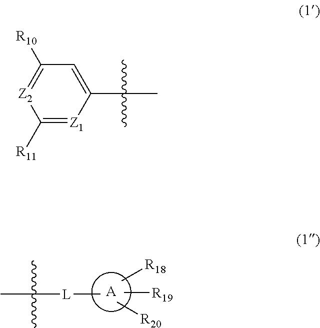

wherein L is —CH(CH₃)— can be prepared, for example, according to Scheme 12, wherein A, Z₁, Z₂, R₁₁, R₁₈, R₁₉ and R₂₀ are as defined above, and X is a halogen. In the method of Scheme 12, the aldehyde compound of formula [4b] is reacted with methyl magnesium bromide in a suitable solvent such as THF to produce hydroxyl compound of [12a], which is then brominated with CBr₄ in a suitable solvent such as DCM in the presence of triphenyl phosphine to produce the compound of formula [12b]. Coupling the compound of formula [4a] with the compound of formula [12b] in a suitable solvent such as DCM in the presence of tetrabutylammonium iodide (TBAI) and DIPEA produces the intermediate of formula [9′].

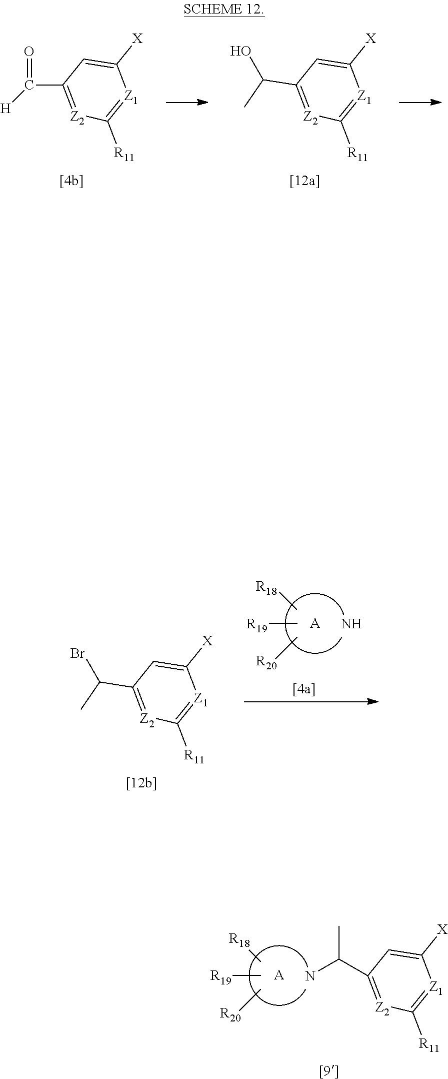

Intermediate compounds of formula [1], wherein R₁ is a group of formula (5)

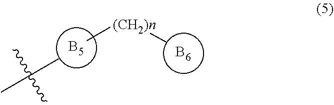

wherein B₅ is phenyl optionally substituted with e.g. chloro substituent, B₆ is a triazolyl ring optionally substituted with one C₁₋₇ alkyl substituent, and n is 0, can be prepared according to Scheme 13, wherein Z, M, R₂ are as defined above, and X is a halogen, for example chloro, bromo or iodo. In the method of Scheme 13, the compound of formula [13a] is first treated with hydrazine hydrate to produce a compound of formula [13b] which is then reacted with methyl isothiocyanate in a suitable solvent such as THF. Treating the obtained compound of formula [13c] with base, such as NaOH, produces the thiol compound of formula [13d]. Treatment with hydrogen peroxide in a suitable solvent such as DCM in the presence of acetic acid produces compound of formula [13e]. Coupling this compound with the isoindolinone compound of formula [13f] in a suitable solvent such as 1,4-dioxane:water mixture in the presence of a base such as K₂CO₃ and a suitable catalyst such as Pd(amphos)Cl₂ produces the intermediate compound of formula [1-a].

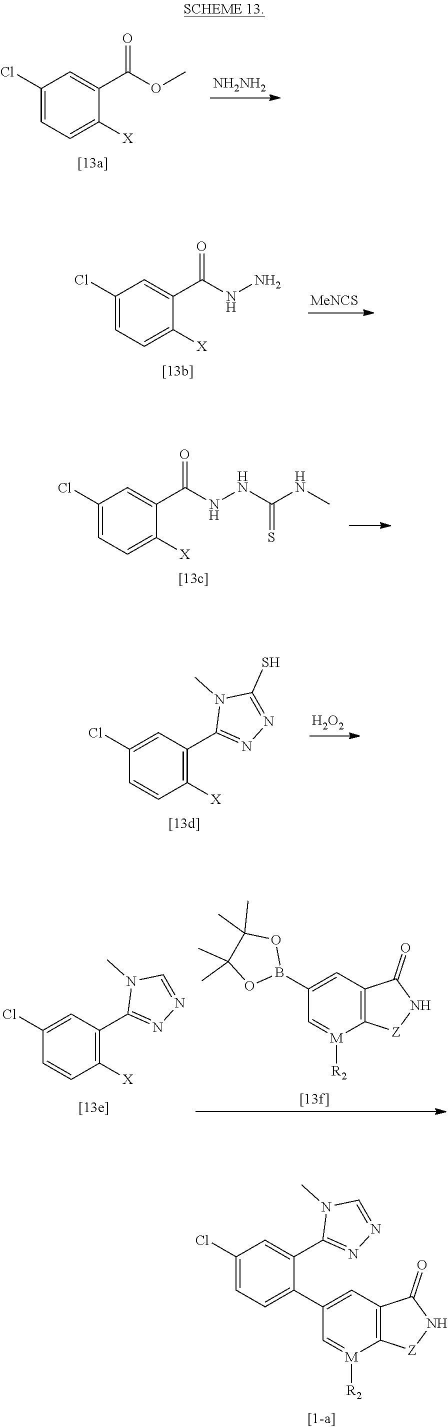

Intermediate compounds of formula [1], wherein R₁ is a group of formula (5)

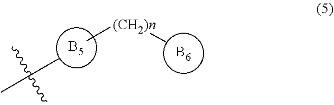

wherein B₅ is optionally substituted phenyl, B₆ is a triazolyl ring optionally substituted with one C₁₋₇ alkyl substituent, and n is 1, can be prepared according to Scheme 14, wherein Z, M, R₂ are as defined above, and X is a halogen, for example chloro, bromo or iodo. In the method of Scheme 14, the compound of formula [14a] is first coupled with the isoindolinone compound of formula [14b] in a suitable solvent such as 1,4-dioxane:water mixture in the presence of a base such as Cs₂CO₃ and a suitable catalyst such as Pd(dppf)Cl₂ to produce the compound of formula [14c]. Treating this compound with hydrazine hydrate produces a compound of formula [14d]. Further reaction with methyl isothiocyanate in a suitable solvent such as THF produces a compound of formula [14e]. Treating the compound of formula [14e] with base, such as NaOH, followed by reaction of the obtained compound of formula [14f] with hydrogen peroxide in a suitable solvent such as DCM in the presence of acetic acid produces the intermediate compound of formula [1-b].

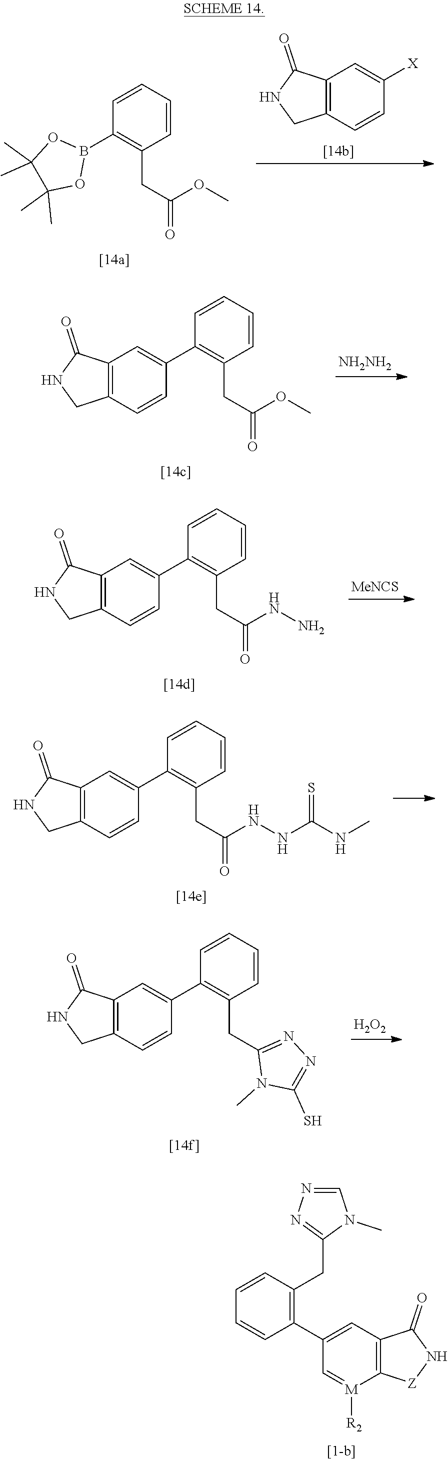

Alternatively, the compounds of formula (I) can be prepared as disclosed in the specific Examples of the present disclosure.

Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “subject”, as employed herein, refers to humans and animals.

The term “halo” or “halogen”, as employed herein as such or as part of another group, refers to chlorine, bromine, fluorine or iodine. Preferred halogens are chlorine and bromine and fluorine.

The term “C₁₋₇ alkyl”, as employed herein as such or as part of another group, refers to a straight or branched chain saturated hydrocarbon group having 1, 2, 3, 4, 5, 6 or 7 carbon atom(s). Representative examples of C₁₋₇ alkyl include, but are not limited to, methyl, ethyl, n-propyl, iso-propyl, n-butyl, iso-butyl, sec-butyl, tert-butyl, n-pentyl, iso-pentyl and n-hexyl. One preferred embodiment of “C₁₋₇ alkyl” is C₁₋₃ alkyl. The term “C₁₋₃ alkyl” refers to an embodiment of “C₁₋₇ alkyl” having 1, 2 or 3 carbon atoms. Examples of “C₁₋₃ alkyl” include, but are not limited to, methyl, ethyl, n-propyl and iso-propyl. One preferred “C₁₋₇ alkyl” is methyl group.

The term “C₂₋₇ alkenyl”, as employed herein as such or as part of another group, refers to an aliphatic hydrocarbon group having 2, 3, 4, 5, 6 or 7 carbon atoms and containing one or several double bonds. Representative examples include, but are not limited to, ethenyl, propenyl and hexenyl. One preferred embodiment of “C₂₋₇ alkenyl” is C₂₋₄ alkenyl. The term “C₂₋₄ alkenyl” refers to a embodiment of “C₂₋₇ alkenyl” having 2, 3 or 4 carbon atoms. Representative examples include, but are not limited to, ethenyl, propenyl and butenyl. One preferred “C₂₋₇ alkenyl” is —CH═CH₂ group.

The term “3-6 membered carbocyclic ring”, as employed herein, refers to a saturated, partially saturated or aromatic ring with 3 to 6 ring atoms consisting of carbon atoms only. Representative examples of a 3-6 membered carbocyclic ring include, but are not limited to, phenyl, cyclohexyl, cyclohexenyl, cyclopentyl, cyclopentenyl and cyclobutyl rings.

The term “C₃₋₇ cycloalkyl”, as employed herein as such or as part of another group, refers to a saturated monocyclic or bicyclic (fused, bridged or in spiro configuration) hydrocarbon ring containing 3, 4, 5, 6 or 7 ring atoms consisting of carbon atoms only. Representative examples of C₃₋₇ cycloalkyl group include, but are not limited to, cyclopropyl, cyclobutyl, cyclopentyl and cyclohexyl and spiro(3,3)heptane. One preferred C₃₋₇ cycloalkyl group is a saturated monocyclic hydrocarbon ring containing 3, 4, 5, 6 or 7 ring atoms consisting of carbon atoms only, for example cyclopropyl group.

The term “C₃₋₆ cycloalkyl C₁₋₇ alkyl”, as employed herein as such or as part of another group, refers to a C₃₋₆ cycloalkyl group as defined herein, appended to the parent molecular moiety through a C₁₋₇ alkyl group, as defined herein.

The term “C₁₋₇ alkyl C₃₋₇ cycloalkyl” as employed herein as such or as part of another group, refers to a C₁₋₇ alkyl group as defined herein, appended to the parent molecular moiety through a C₃₋₇ cycloalkyl group, as defined herein.

The term “halogen C₃₋₇ cycloalkyl” as employed herein as such or as part of another group, refers to at least one halogen, as defined herein, appended to the parent molecular moiety through a C₃₋₇ cycloalkyl group, as defined herein.

The term “halogen C₃₋₇ cycloalkyl C₁₋₇ alkyl” as employed herein as such or as part of another group, refers to at least one halogen, as defined herein, appended to the parent molecular moiety through a C₃₋₇ cycloalkyl C₁₋₇ alkyl group, as defined herein.

The term “hydroxy”, as employed herein as such or as part of another group, refers to an —OH group.

The term “cyano”, as employed herein as such or as part of another group, refers to a —CN group.

The term “carboxy”, as employed herein as such or as part of another group, refers to —COOH group.

The term “carbonyl”, as employed herein as such or as part of another group, refers to a carbon atom double-bonded to an oxygen atom (C═O).

The term “oxo”, as employed herein as such or as part of another group, refers to oxygen atom linked to another atom by a double bond (=O).

The term “C₁₋₇ alkoxy”, as employed herein as such or as part of another group, refers to C₁₋₇ alkyl, as defined herein, appended to the parent molecular moiety through an oxygen atom. Representative examples of C₁₋₇ alkoxy include, but are not limited to methoxy, ethoxy, propoxy, butoxy, isobutoxy, sec-butoxy and tert-butoxy. One preferred embodiment of “C₁₋₇ alkoxy” is C₁₋₃ alkoxy. The term “C₁₋₃ alkoxy” refers to an embodiment of “C₁₋₇ alkoxy” having 1, 2 or 3 carbon atoms. Representative examples of C₁₋₃ alkoxy include, but are not limited to methoxy, ethoxy, propoxy. One preferred “C₁₋₇ alkoxy” group is methoxy.

The term “hydroxy C₁₋₇ alkyl”, as employed herein, refers to at least one hydroxy group, as defined herein, appended to the parent molecular moiety through a C₁₋₇ alkyl group, as defined herein. Representative examples of hydroxy C₁₋₇ alkyl include, but are not limited to, hydroxymethyl, 2,2-dihydroxyethyl, 1-hydroxyethyl, 3-hydroxypropyl, 1-hydroxypropyl, 1-methyl-1-hydroxyethyl and 1-methyl-1-hydroxypropyl.

The term “halogen C₁₋₇ alkyl”, as employed herein, refers to at least one halogen, as defined herein, appended to the parent molecular moiety through a C₁₋₇ alkyl group, as defined herein. Representative examples of halogen C₁₋₇ alkyl include, but are not limited to, fluoromethyl, difluoromethyl, trifluoromethyl, 2-chloroethyl and 3-bromo-propyl. Preferred “halogen C₁₋₇ alkyl” groups are trifluoromethyl, trifluoroethyl and trifluoropropyl.

The term “halogen C₁₋₇ alkoxy”, as employed herein, refers to at least one halogen, as defined herein, appended to the parent molecular moiety through a C₁₋₇ alkoxy group, as defined herein.

The term “C₁₋₇ alkoxy C₁₋₇ alkyl”, as employed herein as such or as part of another group, refers to at least one C₁₋₇ alkoxy group, as defined herein, appended to the parent molecular moiety through a C₁₋₇ alkyl group, as defined herein.

The term “3-6 membered carbocyclic ring”, as employed herein, refers to a saturated, partially saturated or aromatic ring with 3 to 6 ring atoms consisting of carbon atoms only. Representative examples of a 3-6 membered carbocyclic ring include, but are not limited to, phenyl, cyclohexyl, cyclohexenyl, cyclopentyl, cyclopentenyl and cyclobutyl rings.

The term “3-6 membered heterocyclic ring” as employed herein, refers to saturated, partially saturated or aromatic ring with 3-6 ring atoms, of which 1-3 atoms are heteroatoms selected from a group consisting of N, O and S. Representative examples of a 3-6 membered heterocyclic ring include, but are not limited to, oxetanyl, azetidinyl, oxiranyl, pyrazolyl, pyrimidinyl, pyridinyl, piperidinyl, piperazinyl, furanyl, 1,2,4-triazol-3-yl, 1,2,3-triazol-1-yl, 1,2,4-oxadiazolyl, morpholinyl, pyrrolidinyl, thiazolyl, isoxazolyl, pyrazinyl, tetrahydropyranyl, oxazolyl and imidazolyl rings.

The term “5-6 membered heterocyclic ring” as employed herein, refers to saturated, partially saturated or aromatic ring with 5-6 ring atoms, of which 1-4 atoms are heteroatoms selected from a group consisting of N, O and S. Representative examples of a 5-6 membered heterocyclic ring include, but are not limited to, 1,2,4-triazol-3-yl, 1,2,3-triazol-1-yl, 1,2,4-oxadiazolyl, tetrazolyl, pyrazolyl, pyrimidinyl, pyridinyl, piperidinyl, piperazinyl, furanyl, tetrahydrofuranyl, morpholinyl, pyrrolidinyl, thiazolyl, isoxazolyl, pyrazinyl, tetrahydropyranyl, oxazolyl and imidazolyl rings.

The term “5-10 membered monocyclic, bicyclic or spiro bicyclic heterocyclic ring”, as employed herein, refers to a saturated, partially saturated or aromatic monocyclic or bicyclic (fused, bridged or in spiro configuration) ring system with 5 to 10 ring atoms, of which 1-4 atoms are heteroatoms selected from a group consisting of N, O and S. Representative examples of a “5-10 membered monocyclic, bicyclic or spiro bicyclic heterocyclic ring” include, but are not limited to, piperidinyl, pyrrolidinyl, piperazinyl, morpholinyl, imidazolyl, pyrazolyl, 3-azabicyclo[3.1.0]hexan-3-yl, 5-azaspiro[2.4]heptan-5-yl, 2-azaspiro[3.3]heptan-2-yl, 3,4-dihydroisoquinolin-2(1H)-yl, 1-oxa-8-azaspiro[4.5]decan-8-yl, 1-oxa-7-azaspiro[4.4]nonan-7-yl, 2-oxa-6-azaspiro[3.3]heptan-6-yl, hexahydrocyclopenta[c]pyrrol-2(1H)-yl, hexahydropyrrolo-[1,2-a]pyrazin-2(1H)-yl, 2,5-diazabicyclo[2.2.1]heptan-2-yl, 1,5,6,7-tetrahydro-4H-pyrazolo[4,3-b]pyridin-4-yl,1,2,4-triazol-1-yl, 1,2,3-triazol-1-yl, pyrimidinyl, pyridinyl, tetrazolyl, furanyl, tetrahydrofuranyl, thiazolyl, isoxazolyl, pyrazinyl, tetrahydropyranyl, 1,2,4-oxadiazolyl, oxazolyl, indolyl and 4,5-dihydroimidazolyl rings.

The term “spiro bicyclic ring”, as employed herein, refers to a ring system where the two rings have one common carbon atom.

The term “bicyclic ring”, as employed herein, refers to a saturated, partially saturated or aromatic ring system where the two rings are fused (have two common ring atoms) or bridged (share three or more common ring atoms).

The term “substituted” as used herein in connection with various residues refers to, if not otherwise defined, to halogen substituents, such as fluorine, chlorine, bromine, iodine, or C₁₋₇ alkyl, C₃₋₇ cycloalkyl, hydroxy, amino, nitro, cyano, thiol C₁₋₇ alkyl, methylsulfonyl, C₁₋₇ alkoxy, halo C₁₋₇ alkyl, hydroxy C₁₋₇ alkyl or amino C₁₋₇ alkyl substituents. Preferred are halogen, C₁₋₇ alkyl, hydroxy, amino, halo C₁₋₇ alkyl, C₁₋₇ alkoxy and methylsulfonyl substituents. In one group of preferred substituents are 1-2 substituents selected from C₁₋₇ alkyl or halogen substituents, particularly C₁₋₃ alkyl or halogen substituents, particularly methyl, ethyl, chloro, fluoro or bromo substituents.

The “substituted” groups may contain 1 to 3, preferably 1 or 2, of the above mentioned substituents, if not otherwise defined.

Optically active enantiomers or diastereomers of compounds of formula (I) can be prepared e.g. by resolution of the racemic end product by known methods or by using suitable optically active starting materials. Similarly, racemic compounds of formula (I) can be prepared by using racemic starting materials. Resolution of racemic compounds of formula (I) or a racemic starting material thereof can be carried out, for example, by converting the racemic compound into its diastereromeric salt mixture by reaction with an optically active acid and subsequent separation of the diastereomers by crystallization. Representative examples of said optically active acids include, but are not limited to, D-tartaric acid and dibenzoyl-D-tartaric acid. Alternatively, preparative chiral chromatography may be used for resolution of the racemic mixture.

Pharmaceutically acceptable salts are well known in the field of pharmaceuticals. Non-limiting examples of suitable salts include metal salts, ammonium salts, salts with an organic base, salts with an inorganic acid, salts with organic acid, and salts with basic or acidic amino acid. Non-limiting examples of metal salts include alkali metal salts such as sodium salt and potassium salt; alkaline earth metal salts such as calcium salt, and magnesium salt. Non-limiting examples of salts with inorganic or organic acids include chlorides, bromides, sulfates, nitrates, phosphates, sulfonates, methane sulfonates, formates, tartrates, maleates, citrates, benzoates, salicylates, ascorbates, acetates, oxalates, fumarates, hemifumarates, and succinates. Pharmaceutically acceptable esters, when applicable, may be prepared by known methods using pharmaceutically acceptable acids that are conventional in the field of pharmaceuticals and that retain the pharmacological properties of the free form. Non-limiting examples of these esters include esters of aliphatic or aromatic alcohols, e.g. methyl, ethyl, propyl, isopropyl, butyl, isobutyl, sec-butyl, tert-butyl esters. Phosphate esters and carbonate esters, are also within the scope of the invention.

The definition of formula (I) above is inclusive of all the possible isotopes and isomers, such as stereoisomers, of the compounds, including geometric isomers, for example Z and E isomers (cis and trans isomers), and optical isomers, e.g. diastereomers and enantiomers, and prodrug esters, e.g. phosphate esters and carbonate esters.

It will be appreciated by those skilled in the art that the present compounds may contain at least one chiral center. Accordingly, the compounds may exist in optically active or racemic forms. It is to be understood that the formula (I) includes any racemic or optically active form, or mixtures thereof. In one embodiment, the compounds are the pure (R)-isomers. In another embodiment, the compounds are the pure (S)-isomers. In another embodiment, the compounds are a mixture of the (R) and the (S) isomers. In another embodiment, the compounds are a racemic mixture comprising an equal amount of the (R) and the (S) isomers. The compounds may contain two chiral centers. In such case, according to one embodiment, the compounds are a mixture of diasteromers. According to another embodiment, the compounds of the invention are a mixture of enantiomers. According to still another embodiment, the compounds are pure enantiomers. The individual isomers may be obtained using the corresponding isomeric forms of the starting material or they may be separated after the preparation of the end compound according to conventional separation methods. For the separation of optical isomers, e.g. enantiomers or diastereomers, from the mixture thereof the conventional resolution methods, e.g. fractional crystallisation, may be used.

The present compounds may also exist as tautomers or equilibrium mixtures thereof wherein a proton of a compound shifts from one atom to another. Examples of tautomerism include, but are not limited to, amido-imido, keto-enol, phenol-keto, oxime-nitroso, nitro-aci, imine-enamine, annular tautomerism of heterocyclic rings such as pyrozole ring, and the like. Tautomeric forms are intended to be encompassed by compounds of formula (I), even though only one tautomeric form may be depicted.

Examples of preferred compounds of one group of formula (I) include
(S)-6-(3-((4-Methyl-4H-1,2,4-triazol-3-yl)methyl)oxetan-3-yl)-2-(3-((3-methyl-piperidin-1-yl)methyl)-5-(trifluoromethyl)phenyl)isoindolin-1-one (Compound 1);
(S)-6-(3-((4-Methyl-4H-1,2,4-triazol-3-yl)methyl)oxetan-3-yl)-2-(4-((3-methyl-piperidin-1-yl)methyl)-6-(trifluoromethyl)pyridin-2-yl)isoindolin-1-one (Compound 2);
(S)-2-(3-Fluoro-5-((3-methylpiperidin-1-yl)methyl)phenyl)-6-(3-((4-methyl-4H-1,2,4-triazol-3-yl)methyl)oxetan-3-yl)isoindolin-1-one (Compound 3);
(S)-6-(3-((4-Methyl-4H-1,2,4-triazol-3-yl)methyl)oxetan-3-yl)-2-(3-methyl-5-((3-methylpiperidin-1-yl)methyl)phenyl)isoindolin-1-one (Compound 4);
(S)-2-(3-Methoxy-5-((3-methylpiperidin-1-yl)methyl)phenyl)-6-(3-((4-methyl-4H-1,2,4-triazol-3-yl)methyl)oxetan-3-yl)isoindolin-1-one (Compound 5);
(S)-2-(3-Chloro-5-((3-methylpiperidin-1-yl)methyl)phenyl)-6-(3-((4-methyl-4H-1,2,4-triazol-3-yl)methyl)oxetan-3-yl)isoindolin-1-one (Compound 6);
(S)-2-(3-Cyclopropyl-5-((3-methylpiperidin-1-yl)methyl)phenyl)-6-(3-((4-methyl-4H-1,2,4-triazol-3-yl)methyl)oxetan-3-yl)isoindolin-1-one (Compound 7);
(S)-6-(3-((4-Methyl-4H-1,2,4-triazol-3-yl)methyl)oxetan-3-yl)-2-(3-((3-methyl-piperidin-1-yl)methyl)phenyl)isoindolin-1-one (Compound 8);
(S)-6-(3-((4-Methyl-4H-1,2,4-triazol-3-yl)methyl)oxetan-3-yl)-2-(3-((3-methyl-piperidin-1-yl)methyl)-5-vinylphenyl)isoindolin-1-one (Compound 9);
(S)-2-(3-Bromo-5-((3-methylpiperidin-1-yl)methyl)phenyl)-6-(3-((4-methyl-4H-1,2,4-triazol-3-yl)methyl)oxetan-3-yl)isoindolin-1-one (Compound 10);
(S)-2-(3-Ethyl-5-((3-methylpiperidin-1-yl)methyl)phenyl)-6-(3-((4-methyl-4H-1,2,4-triazol-3-yl)methyl)oxetan-3-yl)isoindolin-1-one (Compound 11);
6-(3-((4-Methyl-4H-1,2,4-triazol-3-yl)methyl)oxetan-3-yl)-2-(3-methyl-5-(1-((S)-3-methylpiperidin-1-yl)ethyl)phenyl)isoindolin-1-one (Compound 12);
2-(3-(((Cyclopropylmethyl)amino)methyl)-5-(trifluoromethyl)phenyl)-6-(3-((4-methyl-4H-1,2,4-triazol-3-yl)methyl)oxetan-3-yl)isoindolin-1-one (Compound 13);
(S)-6-(3-((4-Methyl-4H-1,2,4-triazol-3-yl)methyl)oxetan-3-yl)-2-(3-((3-methyl-piperidin-1-yl)methyl)-5-(piperidin-1-yl)phenyl)isoindolin-1-one (Compound 14);
(S)-2-(3-Isopropyl-5-((3-methylpiperidin-1-yl)methyl)phenyl)-6-(3-((4-methyl-4H-1,2,4-triazol-3-yl)methyl)oxetan-3-yl)isoindolin-1-one (Compound 15);
(S)-2-(3-(tert-Butyl)-5-((3-methylpiperidin-1-yl)methyl)phenyl)-6-(3-((4-methyl-4H-1,2,4-triazol-3-yl)methyl)oxetan-3-yl)isoindolin-1-one (Compound 16);
(S)-6-(3-((4-Methyl-4H-1,2,4-triazol-3-yl)methyl)oxetan-3-yl)-2-(2-((3-methyl-piperidin-1-yl)methyl)-6-(trifluoromethyl)pyridin-4-yl)isoindolin-1-one (Compound 17);
2-(3-(((Cyclopropylmethyl)amino)methyl)-5-methylphenyl)-6-(3-((4-methyl-4H-1,2,4-triazol-3-yl)methyl)oxetan-3-yl)isoindolin-1-one (Compound 18);
6-(3-((4-Methyl-4H-1,2,4-triazol-3-yl)methyl)oxetan-3-yl)-2-(3-methyl-5-(((3,3,3-trifluoropropyl)amino)methyl)phenyl)isoindolin-1-one (Compound 19);
2-(3-((((2,2-Difluorocyclopropyl)methyl)amino)methyl)-5-methylphenyl)-6-(3-((4-methyl-4H-1,2,4-triazol-3-yl)methyl)oxetan-3-yl)isoindolin-1-one (Compound 20);
(S)-6-(3-((4-Methyl-4H-1,2,4-triazol-3-yl)methyl)oxetan-3-yl)-2-(3-((3-methyl-piperidin-1-yl)methyl)-5-(pyrrolidin-1-yl)phenyl)isoindolin-1-one (Compound 21);
6-(3-((4-Methyl-4H-1,2,4-triazol-3-yl)methyl)oxetan-3-yl)-2-(3-methyl-5-(((2,2,2-trifluoroethyl)amino)methyl)phenyl)isoindolin-1-one (Compound 22);
(S)-6-(3-((4-Methyl-4H-1,2,4-triazol-3-yl)methyl)oxetan-3-yl)-2-(2-methyl-6-((3-methylpiperidin-1-yl)methyl)pyridin-4-yl)isoindolin-1-one (Compound 23);
(S)-2-(3-(Azetidin-1-yl)-5-((3-methylpiperidin-1-yl)methyl)phenyl)-6-(3-((4-methyl-4H-1,2,4-triazol-3-yl)methyl)oxetan-3-yl)isoindolin-1-one (Compound 24);
6-(3-((4-Methyl-4H-1,2,4-triazol-3-yl)methyl)oxetan-3-yl)-2-(6-(trifluoro-methyl)-4-(((3,3,3-trifluoropropyl)amino)methyl)pyridin-2-yl)isoindolin-1-one (Compound 25);
(S)-2-(6-Methyl-4-((3-methylpiperidin-1-yl)methyl)pyridin-2-yl)-6-(3-((4-methyl-4H-1,2,4-triazol-3-yl)methyl)oxetan-3-yl)isoindolin-1-one (Compound 26);
(S)-6-(3-((4-Methyl-4H-1,2,4-triazol-3-yl)methyl)oxetan-3-yl)-2-(6-(trifluoro-methyl)-4-((3-(trifluoromethyl)piperidin-1-yl)methyl)pyridin-2-yl)isoindolin-1-one (Compound 27);
2-(4-((4,4-Difluoro-3-methylpiperidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 28);
1-((2-(6-(3-((4-Methyl-4H-1,2,4-triazol-3-yl)methyl)oxetan-3-yl)-1-oxoisoindo-lin-2-yl)-6-(trifluoromethyl)pyridin-4-yl)methyl)piperidine-3-carbonitrile (Compound 29);
(S)-2-(4-((3-Hydroxypiperidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 30);
2-(4-((3,4-Dihydroisoquinolin-2(1H)-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 31);
2-(4-((1-Oxa-8-azaspiro[4.5]decan-8-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 32);
2-(4-((3-Fluoropiperidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 33);
(S)-6-(3-((4-Methyl-4H-1,2,4-triazol-3-yl)methyl)oxetan-3-yl)-2-(4-((3-methyl-pyrrolidin-1-yl)methyl)-6-(trifluoromethyl)pyridin-2-yl)isoindolin-1-one (Compound 34);
2-(4-((Hexahydrocyclopenta[c]pyrrol-2(1H)-yl)methyl)-6-(trifluoromethyl)pyri-din-2-yl)-6-(3-((4-methyl-4H-1,2,4-triazol-3-yl)methyl)oxetan-3-yl)isoindolin-1-one (Compound 35);
(S)-2-(4-(3-Hydroxypyrrolidine-1-carbonyl)-6-(trifluoromethyl)pyridin-2-yl)-6-(3-((4-methyl-4H-1,2,4-triazol-3-yl)methyl)oxetan-3-yl)isoindolin-1-one (Compound 36);
2-(4-((3-(Hydroxymethyl)piperidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 37);
6-(3-((4-Methyl-4H-1,2,4-triazol-3-yl)methyl)oxetan-3-yl)-2-(6-(trifluoro-methyl)-4-((4-(trifluoromethyl)piperidin-1-yl)methyl)pyridin-2-yl)isoindolin-1-one (Compound 38);
2-(4-(((3S,5R)-3,5-Dimethylpiperidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 39);
(S)-2-(6-Chloro-4-((3-methylpiperidin-1-yl)methyl)pyridin-2-yl)-6-(3-((4-methyl-4H-1,2,4-triazol-3-yl)methyl)oxetan-3-yl)isoindolin-1-one (Compound 40);
(R)-2-(4-((Hexahydropyrrolo[1,2-a]pyrazin-2(1H)-yl)methyl)-6-(trifluoro-methyl)pyridin-2-yl)-6-(3-((4-methyl-4H-1,2,4-triazol-3-yl)methyl)oxetan-3-yl)iso-indolin-1-one (Compound 41);
2-(4-(((3S,5S)-3,5-Dimethylpiperidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 42);
2-(4-((4-Hydroxypiperidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 43);
2-(4-((6-Azaspiro[2.5]octan-6-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 44);
6-(3-((4-Methyl-4H-1,2,4-triazol-3-yl)methyl)oxetan-3-yl)-2-(4-((2-methyl-piperidin-1-yl)methyl)-6-(trifluoromethyl)pyridin-2-yl)isoindolin-1-one (Compound 45);
(R)-6-(3-((4-Methyl-4H-1,2,4-triazol-3-yl)methyl)oxetan-3-yl)-2-(4-((2-methyl-morpholino)methyl)-6-(trifluoromethyl)pyridin-2-yl)isoindolin-1-one (Compound 46);
2-(4-((3,3-Difluoropiperidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 47);
2-(4-((4,4-Difluoropiperidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 48);
(S)-2-(3-(Cyclopropylmethyl)-5-((3-methylpiperidin-1-yl)methyl)phenyl)-6-(3-((4-methyl-4H-1,2,4-triazol-3-yl)methyl)oxetan-3-yl)isoindolin-1-one (Compound 49);
2-(4-((1-Oxa-7-azaspiro[4.4]nonan-7-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 50);
2-(4-((2-Azaspiro[3.3]heptan-2-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 51);
2-(6-Chloro-4-(((3S,5R)-3,5-dimethylpiperidin-1-yl)methyl)pyridin-2-yl)-6-(3-((4-methyl-4H-1,2,4-triazol-3-yl)methyl)oxetan-3-yl)isoindolin-1-one (Compound 52);
(S)-6-(3-((4-Methyl-4H-1,2,4-triazol-3-yl)methyl)oxetan-3-yl)-2-(2-methyl-6-((3-methylpiperidin-1-yl)methyl)pyrimidin-4-yl)isoindolin-1-one (Compound 53);
2-(4-((2-Methyl-1H-imidazol-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 54);
2-(4-((2-Oxa-6-azaspiro[3.3]heptan-6-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 55);
2-(3-(((3S,5R)-3,5-Dimethylpiperidin-1-yl)methyl)-5-methylphenyl)-6-(3-((4-methyl-4H-1,2,4-triazol-3-yl)methyl)oxetan-3-yl)isoindolin-1-one (Compound 56);
2-(6-Chloro-4-(((cyclopropylmethyl)amino)methyl)pyridin-2-yl)-6-(3-((4-methyl-4H-1,2,4-triazol-3-yl)methyl)oxetan-3-yl)isoindolin-1-one (Compound 57);
2-(6-Chloro-4-(((S)-3-methylpiperidin-1-yl)methyl)pyridin-2-yl)-6-((1s,3R)-3-methyl-1-(4-methyl-4H-1,2,4-triazol-3-yl)cyclobutyl)isoindolin-1-one (Compound 58);
2-(3-(((3S,5S)-3,5-Dimethylpiperidin-1-yl)methyl)-5-methylphenyl)-6-(3-((4-methyl-4H-1,2,4-triazol-3-yl)methyl)oxetan-3-yl)isoindolin-1-one (Compound 59);
2-(6-Chloro-4-(((3S,5S)-3,5-dimethylpiperidin-1-yl)methyl)pyridin-2-yl)-6-(3-((4-methyl-4H-1,2,4-triazol-3-yl)methyl)oxetan-3-yl)isoindolin-1-one (Compound 60);
(S)-2-(4-((3-Fluoropyrrolidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 61);
2-(4-((5-Azaspiro[2.4]heptan-5-yl)methyl)-6-chloropyridin-2-yl)-6-(3-((4-methyl-4H-1,2,4-triazol-3-yl)methyl)oxetan-3-yl)isoindolin-1-one (Compound 62);
2-(4-((4-Methyl-3-oxopiperazin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 63);
2-(6-Chloro-4-(piperidin-1-ylmethyl)pyridin-2-yl)-6-(3-((4-methyl-4H-1,2,4-triazol-3-yl)methyl)oxetan-3-yl)isoindolin-1-one (Compound 64);
2-(3-(((3S,5R)-3,5-Dimethylpiperidin-1-yl)methyl)-5-(trifluoromethyl)phenyl)-6-(3-((4-methyl-4H-1,2,4-triazol-3-yl)methyl)oxetan-3-yl)isoindolin-1-one (Compound 65);
2-(6-Chloro-4-(((3,3,3-trifluoropropyl)amino)methyl)pyridin-2-yl)-6-(3-((4-methyl-4H-1,2,4-triazol-3-yl)methyl)oxetan-3-yl)isoindolin-1-one (Compound 66);
(S)-2-(6-Chloro-4-((3-methylpyrrolidin-1-yl)methyl)pyridin-2-yl)-6-(3-((4-methyl-4H-1,2,4-triazol-3-yl)methyl)oxetan-3-yl)isoindolin-1-one (Compound 67);
(R)-2-(6-Chloro-4-((3-methylpyrrolidin-1-yl)methyl)pyridin-2-yl)-6-(3-((4-methyl-4H-1,2,4-triazol-3-yl)methyl)oxetan-3-yl)isoindolin-1-one (Compound 68);
2-(4-((5-Methyl-1H-imidazol-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 69);
2-(4-((4-Methyl-1H-imidazol-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 70);
2-(4-((3-Azabicyclo[3.1.0]hexan-3-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 71);
2-(4-(((2R,6S)-2,6-Dimethylmorpholino)methyl)-6-(trifluoromethyl)pyridin-2-yl)-6-(3-((4-methyl-4H-1,2,4-triazol-3-yl)methyl)oxetan-3-yl)isoindolin-1-one (Compound 72);
(S)-2-(6-Cyclopropyl-4-((3-methylpiperidin-1-yl)methyl)pyridin-2-yl)-6-(3-((4-methyl-4H-1,2,4-triazol-3-yl)methyl)oxetan-3-yl)isoindolin-1-one (Compound 73);
2-(4-(((3,3-Difluorocyclobutyl)amino)methyl)-6-(trifluoromethyl)pyridin-2-yl)-6-(3-((4-methyl-4H-1,2,4-triazol-3-yl)methyl)oxetan-3-yl)isoindolin-1-one (Compound 74);
2-(3-(2-(Ethylamino)ethyl)-5-(trifluoromethyl)phenyl)-6-(3-((4-methyl-4H-1,2,4-triazol-3-yl)methyl)oxetan-3-yl)isoindolin-1-one (Compound 75);
2-(1-(2-(Dimethylamino)ethyl)-4-methyl-1H-indazol-6-yl)-6-(3-((4-methyl-4H-1,2,4-triazol-3-yl)methyl)oxetan-3-yl)isoindolin-1-one (Compound 76);
2-(2-Ethyl-5-(trifluoromethyl)-1,2,3,4-tetrahydroisoquinolin-7-yl)-6-(3-((4-methyl-4H-1,2,4-triazol-3-yl)methyl)oxetan-3-yl)isoindolin-1-one (Compound 77);
2-(4-(((3S,5R)-3,5-Dimethylpiperidin-1-yl)methyl)-6-methylpyridin-2-yl)-6-(3-((4-methyl-4H-1,2,4-triazol-3-yl)methyl)oxetan-3-yl)isoindolin-1-one (Compound 78);
2-(6-Chloro-4-((diethylamino)methyl)pyridin-2-yl)-6-(3-((4-methyl-4H-1,2,4-triazol-3-yl)methyl)oxetan-3-yl)isoindolin-1-one (Compound 79);
2-(4-(((Cyclopropylmethyl)amino)methyl)-6-(trifluoromethyl)pyridin-2-yl)-6-(3-((4-methyl-4H-1,2,4-triazol-3-yl)methyl)oxetan-3-yl)isoindolin-1-one (Compound 80);
2-(4-((5-Methyl-2,5-diazabicyclo[2.2.1]heptan-2-yl)methyl)-6-(trifluoromethyl)-pyridin-2-yl)-6-(3-((4-methyl-4H-1,2,4-triazol-3-yl)methyl)oxetan-3-yl)isoindolin-1-one (Compound 81);
2-(4-(((3S,5R)-3,5-Dimethylpiperidin-1-yl)methyl)-6-(trifluoromethyl)pyridin-2-yl)-6-(1-((4-methyl-4H-1,2,4-triazol-3-yl)methyl)cyclobutyl)isoindolin-1-one (Compound 82);
2-(6-Chloro-4-(((S)-3-methylpiperidin-1-yl)methyl)pyridin-2-yl)-6-(1-(4-methyl-4H-1,2,4-triazol-3-yl)propan-2-yl)isoindolin-1-one (Compound 83);
2-(6-Chloro-4-(((3S,5R)-3,5-dimethylpiperidin-1-yl)methyl)pyridin-2-yl)-6-(1-(4-methyl-4H-1,2,4-triazol-3-yl)propan-2-yl)isoindolin-1-one (Compound 84);
2-(6-Chloro-4-(((3S,5R)-3,5-dimethylpiperidin-1-yl)methyl)pyridin-2-yl)-6-(1-(4-methyl-4H-1,2,4-triazol-3-yl)propan-2-yl)isoindolin-1-one (Compound 85);
2-(4-(((3S,5R)-3,5-Dimethylpiperidin-1-yl)methyl)-6-(trifluoromethyl)pyridin-2-yl)-6-((S)-1-(4-methyl-4H-1,2,4-triazol-3-yl)propan-2-yl)isoindolin-1-one (Compound 86);
2-(4-(((3S,5R)-3,5-Dimethylpiperidin-1-yl)methyl)-6-(trifluoromethyl)pyridin-2-yl)-6-((R)-1-(4-methyl-4H-1,2,4-triazol-3-yl)propan-2-yl)isoindolin-1-one (Compound 87);
6-(3-((4-Methyl-4H-1,2,4-triazol-3-yl)methyl)oxetan-3-yl)-2-(4-(((1-methyl-cyclobutyl)amino)methyl)-6-(trifluoromethyl)pyridin-2-yl)isoindolin-1-one (Compound 88);
(S)-2-(6-Chloro-4-((3-methylpiperidin-1-yl)methyl)pyridin-2-yl)-6-(1-((4-methyl-4H-1,2,4-triazol-3-yl)methyl)cyclobutyl)isoindolin-1-one (Compound 89);
2-(6-Chloro-4-(((S)-3-methylpiperidin-1-yl)methyl)pyridin-2-yl)-6-((R)-1-(4-methyl-4H-1,2,4-triazol-3-yl)propan-2-yl)isoindolin-1-one (Compound 90);
2-(6-Chloro-4-(((S)-3-methylpiperidin-1-yl)methyl)pyridin-2-yl)-6-((S)-1-(4-methyl-4H-1,2,4-triazol-3-yl)propan-2-yl)isoindolin-1-one (Compound 91);
2-(4-((1-Methyl-1,5,6,7-tetrahydro-4H-pyrazolo[4,3-b]pyridin-4-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 92);
2-(4-((2,5-Dimethylpiperidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 93);
(S)-6-(3-((4-Methyl-4H-1,2,4-triazol-3-yl)methyl)oxetan-3-yl)-2-(4-((3-methyl-piperidin-1-yl)methyl)benzofuran-6-yl)isoindolin-1-one (Compound 94);
2-(4-(1H-Pyrazol-4-yl)-6-(trifluoromethyl)pyridin-2-yl)-6-(3-((4-methyl-4H-1,2,4-triazol-3-yl)methyl)oxetan-3-yl)isoindolin-1-one (Compound 95);
(R)-6-(3-((4-Methyl-4H-1,2,4-triazol-3-yl)methyl)oxetan-3-yl)-2-(4-((3-methyl-piperazin-1-yl)methyl)-6-(trifluoromethyl)pyridin-2-yl)isoindolin-1-one (Compound 96);
(R)-2-(4-((3,4-Dimethylpiperazin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 97);
2-(4-(((3R,5S)-3,5-Dimethylpiperazin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 98);
2-(4-(13-Amino-5,8,11-trioxa-2-azatridecyl)-6-chloropyridin-2-yl)-6-(3-((4-methyl-4H-1,2,4-triazol-3-yl) methyl)oxetan-3-yl)isoindolin-1-one (Compound 99);
2-(3-(1-Hydroxy-2-(isopropylamino) ethyl)-5-(trifluoromethyl)phenyl)-6-(3-((4-methyl-4H-1,2,4-triazol-3-yl)methyl)oxetan-3-yl)isoindolin-1-one (Compound 100);
6-(4-Chloro-2-(4-methyl-4H-1,2,4-triazol-3-yl)phenyl)-2-(4-(((cyclopropyl-methyl)amino)methyl)-6-methylpyridin-2-yl)isoindolin-1-one (Compound 101);
6-(4-Chloro-2-(4-methyl-4H-1,2,4-triazol-3-yl)phenyl)-2-(6-chloro-4-(((cyclo-propylmethyl)amino)methyl)pyridin-2-yl)isoindolin-1-one (Compound 102);
6-(4-Chloro-2-(4-methyl-4H-1,2,4-triazol-3-yl) phenyl)-2-(4-(1-((cyclopropyl-methyl)amino)ethyl)-6-methylpyridin-2-yl)isoindolin-1-one (Compound 103);
6-(4-Chloro-2-(4-methyl-4H-1,2,4-triazol-3-yl)phenyl)-2-(6-chloro-4-(1-(cyclo-pentylamino)ethyl)pyridin-2-yl)isoindolin-1-one [enantiomer 1](Compound 104);
6-(4-Chloro-2-(4-methyl-4H-1,2,4-triazol-3-yl)phenyl)-2-(6-chloro-4-(1-(cyclo-pentylamino)ethyl)pyridin-2-yl)isoindolin-1-one, enantiomer 2 (Compound 105);
6-(4-Chloro-2-(4-methyl-4H-1,2,4-triazol-3-yl)phenyl)-2-(6-chloro-4-((cyclo-pentylamino)methyl)pyridin-2-yl)isoindolin-1-one (Compound 106);
6-(4-Chloro-2-(4-methyl-4H-1,2,4-triazol-3-yl)phenyl)-2-(6-chloro-4-(1-((cyclo-butylmethyl)amino)ethyl)pyridin-2-yl)isoindolin-1-one (Compound 107);
6-(4-Chloro-2-(4-methyl-4H-1,2,4-triazol-3-yl)phenyl)-2-(6-chloro-4-(((1-cyclo-propylethyl)amino)methyl)pyridin-2-yl)isoindolin-1-one [enantiomer 1](Compound 108);
6-(4-Chloro-2-(4-methyl-4H-1,2,4-triazol-3-yl)phenyl)-2-(6-chloro-4-(((1-cyclo-propylethyl)amino)methyl)pyridin-2-yl)isoindolin-1-one [enantiomer 2](Compound 109);
6-(4-Chloro-2-(4-methyl-4H-1,2,4-triazol-3-yl)phenyl)-2-(6-chloro-4-(1-((cyclo-propylmethyl)amino)ethyl)pyridin-2-yl)isoindolin-1-one [enantiomer 1](Compound 110);
6-(4-Chloro-2-(4-methyl-4H-1,2,4-triazol-3-yl)phenyl)-2-(6-chloro-4-(1-((cyclo-propylmethyl)amino)ethyl)pyridin-2-yl)isoindolin-1-one [enantiomer 2](Compound 111);
(S)-4-(2-(6-Chloro-4-((3-methylpiperidin-1-yl)methyl)pyridin-2-yl)-3-oxoiso-indolin-5-yl)-3-(4-methyl-4H-1,2,4-triazol-3-yl)benzonitrile (Compound 112);
(S)-6-(4-Chloro-2-(4-methyl-4H-1,2,4-triazol-3-yl)phenyl)-2-(6-chloro-4-((3-methylpiperidin-1-yl)methyl)pyridin-2-yl)isoindolin-1-one (Compound 113);
(S)-2-(6-Chloro-4-((3-methylpiperidin-1-yl)methyl)pyridin-2-yl)-6-(1-methyl-4-(4-methyl-4H-1,2,4-triazol-3-yl)-1H-pyrazol-5-yl)isoindolin-1-one (Compound 114);
(S)-2-(6-Chloro-4-((3-methylpiperidin-1-yl)methyl)pyridin-2-yl)-6-(2-((4-methyl-4H-1,2,4-triazol-3-yl)methyl)phenyl)isoindolin-1-one (Compound 115);
2-(6-Chloro-4-(((3,3,3-trifluoropropyl)amino)methyl)pyridin-2-yl)-6-(2-(4-methyl-4H-1,2,4-triazol-3-yl)phenyl)isoindolin-1-one (Compound 116);
2-(4-(((3S,5R)-3,5-Dimethylpiperidin-1-yl)methyl)-6-(trifluoromethyl)pyridin-2-yl)-6-(2-(4-methyl-4H-1,2,4-triazol-3-yl)phenyl)isoindolin-1-one (Compound 117);
(S)-2-(6-Chloro-4-((3-methylpiperidin-1-yl)methyl)pyridin-2-yl)-6-(2-(4-methyl-4H-1,2,4-triazol-3-yl)phenyl)isoindolin-1-one (Compound 118);
6-(4-Chloro-2-(4-methyl-4H-1,2,4-triazol-3-yl)phenyl)-2-(3-chloro-5-(((cyclo-propylmethyl)amino)methyl)phenyl)isoindolin-1-one (Compound 119);
2-(4-(((Cyclopropylmethyl)amino)methyl)-6-methylpyridin-2-yl)-6-(4-methyl-3-(4-methyl-4H-1,2,4-triazol-3-yl)phenyl)isoindolin-1-one (Compound 120);
6-(4-Chloro-2-(4-methyl-4H-1,2,4-triazol-3-yl)phenyl)-2-(6-chloro-4-(1-(((3,3-difluorocyclobutyl)methyl)amino)ethyl)pyridin-2-yl)isoindolin-1-one (Compound 121);
6-(4-Chloro-2-(4-methyl-4H-1,2,4-triazol-3-yl)phenyl)-2-(4-(((1-cyclopropyl-ethyl)amino)methyl)-6-methylpyridin-2-yl)isoindolin-1-one (Compound 122);
6-(4-Chloro-2-(4-methyl-4H-1,2,4-triazol-3-yl)phenyl)-2-(6-chloro-4-((((tetra-hydrofuran-2-yl)methyl)amino)methyl)pyridin-2-yl)isoindolin-1-one [enantiomer 1](Compound 123);
6-(4-Chloro-2-(4-methyl-4H-1,2,4-triazol-3-yl)phenyl)-2-(6-chloro-4-((((tetra-hydrofuran-2-yl)methyl)amino)methyl)pyridin-2-yl)isoindolin-1-one [enantiomer 2](Compound 124);
6-(4-Chloro-2-(4-methyl-4H-1,2,4-triazol-3-yl)phenyl)-2-(4-(1-(cyclopentyl-amino)ethyl)-6-methylpyridin-2-yl)isoindolin-1-one [enantiomer 1](Compound 125);
6-(4-Chloro-2-(4-methyl-4H-1,2,4-triazol-3-yl)phenyl)-2-(4-(1-(cyclopentyl-amino)ethyl)-6-methylpyridin-2-yl)isoindolin-1-one [enantiomer 2](Compound 126);
2-(4-(((Cyclopropylmethyl)amino)methyl)-6-methylpyridin-2-yl)-6-(4-fluoro-2-(4-methyl-4H-1,2,4-triazol-3-yl)phenyl)isoindolin-1-one (Compound 127);
and tautomers and pharmaceutically acceptable salts thereof.
Examples of particularly preferred compounds of one group of formula (I) include
(S)-6-(3-((4-Methyl-4H-1,2,4-triazol-3-yl)methyl)oxetan-3-yl)-2-(4-((3-methyl-piperidin-1-yl)methyl)-6-(trifluoromethyl)pyridin-2-yl)isoindolin-1-one (Compound 2);
(S)-2-(3-Fluoro-5-((3-methylpiperidin-1-yl)methyl)phenyl)-6-(3-((4-methyl-4H-1,2,4-triazol-3-yl)methyl)oxetan-3-yl)isoindolin-1-one (Compound 3);
(S)-6-(3-((4-Methyl-4H-1,2,4-triazol-3-yl)methyl)oxetan-3-yl)-2-(3-methyl-5-((3-methylpiperidin-1-yl)methyl)phenyl)isoindolin-1-one (Compound 4);
(S)-2-(3-Chloro-5-((3-methylpiperidin-1-yl)methyl)phenyl)-6-(3-((4-methyl-4H-1,2,4-triazol-3-yl)methyl)oxetan-3-yl)isoindolin-1-one (Compound 6);
(S)-2-(3-Bromo-5-((3-methylpiperidin-1-yl)methyl)phenyl)-6-(3-((4-methyl-4H-1,2,4-triazol-3-yl)methyl)oxetan-3-yl)isoindolin-1-one (Compound 10);
6-(3-((4-Methyl-4H-1,2,4-triazol-3-yl)methyl)oxetan-3-yl)-2-(3-methyl-5-(1-((S)-3-methylpiperidin-1-yl)ethyl)phenyl)isoindolin-1-one (Compound 12);
2-(3-(((Cyclopropylmethyl)amino)methyl)-5-methylphenyl)-6-(3-((4-methyl-4H-1,2,4-triazol-3-yl)methyl)oxetan-3-yl)isoindolin-1-one (Compound 18);
6-(3-((4-Methyl-4H-1,2,4-triazol-3-yl)methyl)oxetan-3-yl)-2-(3-methyl-5-(((3,3,3-trifluoropropyl)amino)methyl)phenyl)isoindolin-1-one (Compound 19);
6-(3-((4-Methyl-4H-1,2,4-triazol-3-yl)methyl)oxetan-3-yl)-2-(6-(trifluoro-methyl)-4-(((3,3,3-trifluoropropyl)amino)methyl)pyridin-2-yl)isoindolin-1-one (Compound 25);
(S)-2-(6-Methyl-4-((3-methylpiperidin-1-yl)methyl)pyridin-2-yl)-6-(3-((4-methyl-4H-1,2,4-triazol-3-yl)methyl)oxetan-3-yl)isoindolin-1-one (Compound 26);
(S)-6-(3-((4-Methyl-4H-1,2,4-triazol-3-yl)methyl)oxetan-3-yl)-2-(6-(trifluoro-methyl)-4-((3-(trifluoromethyl)piperidin-1-yl)methyl)pyridin-2-yl)isoindolin-1-one (Compound 27);
2-(4-(((3S,5R)-3,5-Dimethylpiperidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 39);
(S)-2-(6-Chloro-4-((3-methylpiperidin-1-yl)methyl)pyridin-2-yl)-6-(3-((4-methyl-4H-1,2,4-triazol-3-yl)methyl)oxetan-3-yl)isoindolin-1-one (Compound 40);
2-(4-(((3S,5S)-3,5-Dimethylpiperidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 42);
2-(6-Chloro-4-(((3S,5R)-3,5-dimethylpiperidin-1-yl)methyl)pyridin-2-yl)-6-(3-((4-methyl-4H-1,2,4-triazol-3-yl)methyl)oxetan-3-yl)isoindolin-1-one (Compound 52);
(S)-6-(3-((4-Methyl-4H-1,2,4-triazol-3-yl)methyl)oxetan-3-yl)-2-(2-methyl-6-((3-methylpiperidin-1-yl)methyl)pyrimidin-4-yl)isoindolin-1-one (Compound 53);
2-(6-Chloro-4-(((cyclopropylmethyl)amino)methyl)pyridin-2-yl)-6-(3-((4-methyl-4H-1,2,4-triazol-3-yl)methyl)oxetan-3-yl)isoindolin-1-one (Compound 57);
2-(6-Chloro-4-(((3S,5S)-3,5-dimethylpiperidin-1-yl)methyl)pyridin-2-yl)-6-(3-((4-methyl-4H-1,2,4-triazol-3-yl)methyl)oxetan-3-yl)isoindolin-1-one (Compound 60);
2-(6-Chloro-4-(piperidin-1-ylmethyl)pyridin-2-yl)-6-(3-((4-methyl-4H-1,2,4-triazol-3-yl)methyl)oxetan-3-yl)isoindolin-1-one (Compound 64);
2-(6-Chloro-4-(((3,3,3-trifluoropropyl)amino)methyl)pyridin-2-yl)-6-(3-((4-methyl-4H-1,2,4-triazol-3-yl)methyl)oxetan-3-yl)isoindolin-1-one (Compound 66);
2-(4-(((3S,5R)-3,5-Dimethylpiperidin-1-yl)methyl)-6-methylpyridin-2-yl)-6-(3-((4-methyl-4H-1,2,4-triazol-3-yl)methyl)oxetan-3-yl)isoindolin-1-one (Compound 78);
2-(4-(((Cyclopropylmethyl)amino)methyl)-6-(trifluoromethyl)pyridin-2-yl)-6-(3-((4-methyl-4H-1,2,4-triazol-3-yl)methyl)oxetan-3-yl)isoindolin-1-one (Compound 80);
2-(4-(((3S,5R)-3,5-Dimethylpiperidin-1-yl)methyl)-6-(trifluoromethyl)pyridin-2-yl)-6-(1-((4-methyl-4H-1,2,4-triazol-3-yl)methyl)cyclobutyl)isoindolin-1-one (Compound 82);
2-(6-Chloro-4-(((S)-3-methylpiperidin-1-yl)methyl)pyridin-2-yl)-6-(1-(4-methyl-4H-1,2,4-triazol-3-yl)propan-2-yl)isoindolin-1-one (Compound 83);
2-(6-Chloro-4-(((3S,5R)-3,5-dimethylpiperidin-1-yl)methyl)pyridin-2-yl)-6-(1-(4-methyl-4H-1,2,4-triazol-3-yl)propan-2-yl)isoindolin-1-one (Compound 85);
2-(4-(((3S,5R)-3,5-Dimethylpiperidin-1-yl)methyl)-6-(trifluoromethyl)pyridin-2-yl)-6-((S)-1-(4-methyl-4H-1,2,4-triazol-3-yl)propan-2-yl)isoindolin-1-one (Compound 86);
(S)-2-(6-Chloro-4-((3-methylpiperidin-1-yl)methyl)pyridin-2-yl)-6-(1-((4-methyl-4H-1,2,4-triazol-3-yl)methyl)cyclobutyl)isoindolin-1-one (Compound 89);
2-(6-Chloro-4-(((S)-3-methylpiperidin-1-yl)methyl)pyridin-2-yl)-6-((R)-1-(4-methyl-4H-1,2,4-triazol-3-yl)propan-2-yl)isoindolin-1-one (Compound 90);
2-(6-Chloro-4-(((S)-3-methylpiperidin-1-yl)methyl)pyridin-2-yl)-6-((S)-1-(4-methyl-4H-1,2,4-triazol-3-yl)propan-2-yl)isoindolin-1-one (Compound 91);
(S)-6-(3-((4-Methyl-4H-1,2,4-triazol-3-yl)methyl)oxetan-3-yl)-2-(4-((3-methyl-piperidin-1-yl)methyl)benzofuran-6-yl)isoindolin-1-one (Compound 94);
6-(4-Chloro-2-(4-methyl-4H-1,2,4-triazol-3-yl)phenyl)-2-(4-(((cyclopropyl-methyl)amino)methyl)-6-methylpyridin-2-yl)isoindolin-1-one (Compound 101);
6-(4-Chloro-2-(4-methyl-4H-1,2,4-triazol-3-yl)phenyl)-2-(6-chloro-4-(((cyclo-propylmethyl)amino)methyl)pyridin-2-yl)isoindolin-1-one (Compound 102);
6-(4-Chloro-2-(4-methyl-4H-1,2,4-triazol-3-yl) phenyl)-2-(4-(1-((cyclopropyl-methyl)amino)ethyl)-6-methylpyridin-2-yl)isoindolin-1-one (Compound 103);
(R)-6-(4-Chloro-2-(4-methyl-4H-1,2,4-triazol-3-yl)phenyl)-2-(6-chloro-4-(1-(cyclopentylamino)ethyl)pyridin-2-yl)isoindolin-1-one (Compound 104);
(S)-6-(4-Chloro-2-(4-methyl-4H-1,2,4-triazol-3-yl)phenyl)-2-(6-chloro-4-(1-(cyclopentylamino)ethyl)pyridin-2-yl)isoindolin-1-one (Compound 105);
6-(4-Chloro-2-(4-methyl-4H-1,2,4-triazol-3-yl)phenyl)-2-(6-chloro-4-(1-((cyclo-butylmethyl)amino)ethyl)pyridin-2-yl)isoindolin-1-one (Compound 107);
6-(4-Chloro-2-(4-methyl-4H-1,2,4-triazol-3-yl)phenyl)-2-(6-chloro-4-(((1-cyclo-propylethyl)amino)methyl)pyridin-2-yl)isoindolin-1-one [enantiomer 1](Compound 108);
6-(4-Chloro-2-(4-methyl-4H-1,2,4-triazol-3-yl)phenyl)-2-(6-chloro-4-(((1-cyclo-propylethyl)amino)methyl)pyridin-2-yl)isoindolin-1-one [enantiomer 2](Compound 109);
6-(4-Chloro-2-(4-methyl-4H-1,2,4-triazol-3-yl)phenyl)-2-(6-chloro-4-(1-((cyclo-propylmethyl)amino)ethyl)pyridin-2-yl)isoindolin-1-one [enantiomer 1](Compound 110);
6-(4-Chloro-2-(4-methyl-4H-1,2,4-triazol-3-yl)phenyl)-2-(6-chloro-4-(1-((cyclo-propylmethyl)amino)ethyl)pyridin-2-yl)isoindolin-1-one [enantiomer 2](Compound 111);
(S)-4-(2-(6-Chloro-4-((3-methylpiperidin-1-yl)methyl)pyridin-2-yl)-3-oxoiso-indolin-5-yl)-3-(4-methyl-4H-1,2,4-triazol-3-yl)benzonitrile (Compound 112);
(S)-6-(4-Chloro-2-(4-methyl-4H-1,2,4-triazol-3-yl)phenyl)-2-(6-chloro-4-((3-methylpiperidin-1-yl)methyl)pyridin-2-yl)isoindolin-1-one (Compound 113);
2-(6-Chloro-4-(((3,3,3-trifluoropropyl)amino)methyl)pyridin-2-yl)-6-(2-(4-methyl-4H-1,2,4-triazol-3-yl)phenyl)isoindolin-1-one (Compound 116);
(S)-2-(6-Chloro-4-((3-methylpiperidin-1-yl)methyl)pyridin-2-yl)-6-(2-(4-methyl-4H-1,2,4-triazol-3-yl)phenyl)isoindolin-1-one (Compound 118);
6-(4-Chloro-2-(4-methyl-4H-1,2,4-triazol-3-yl)phenyl)-2-(3-chloro-5-(((cyclo-propylmethyl)amino)methyl)phenyl)isoindolin-1-one (Compound 119);
6-(4-Chloro-2-(4-methyl-4H-1,2,4-triazol-3-yl)phenyl)-2-(6-chloro-4-(1-(((3,3-difluorocyclobutyl)methyl)amino)ethyl)pyridin-2-yl)isoindolin-1-one (Compound 121);
6-(4-Chloro-2-(4-methyl-4H-1,2,4-triazol-3-yl)phenyl)-2-(4-(((1-cyclopropyl-ethyl)amino)methyl)-6-methylpyridin-2-yl)isoindolin-1-one (Compound 122);
6-(4-Chloro-2-(4-methyl-4H-1,2,4-triazol-3-yl)phenyl)-2-(6-chloro-4-((((tetra-hydrofuran-2-yl)methyl)amino)methyl)pyridin-2-yl)isoindolin-1-one [enantiomer 1](Compound 123);
6-(4-Chloro-2-(4-methyl-4H-1,2,4-triazol-3-yl)phenyl)-2-(6-chloro-4-((((tetra-hydrofuran-2-yl)methyl)amino)methyl)pyridin-2-yl)isoindolin-1-one [enantiomer 2](Compound 124);
6-(4-Chloro-2-(4-methyl-4H-1,2,4-triazol-3-yl)phenyl)-2-(4-(1-(cyclopentyl-amino)ethyl)-6-methylpyridin-2-yl)isoindolin-1-one [enantiomer 1](Compound 125);
6-(4-Chloro-2-(4-methyl-4H-1,2,4-triazol-3-yl)phenyl)-2-(4-(1-(cyclopentyl-amino)ethyl)-6-methylpyridin-2-yl)isoindolin-1-one [enantiomer 2](Compound 126);
2-(4-(((Cyclopropylmethyl)amino)methyl)-6-methylpyridin-2-yl)-6-(4-fluoro-2-(4-methyl-4H-1,2,4-triazol-3-yl)phenyl)isoindolin-1-one (Compound 127);
2-(6-Chloro-4-(((1-cyclopropylethyl)amino)methyl)pyridin-2-yl)-6-(3-((4-methyl-4H-1,2,4-triazol-3-yl)methyl)oxetan-3-yl)isoindolin-1-one (Compound 131);
6-(4-Chloro-2-(4-methyl-4H-1,2,4-triazol-3-yl)phenyl)-2-(4-(((cyclopropyl-methyl)amino)methyl)-6-(trifluoromethyl)pyridin-2-yl)isoindolin-1-one (Compound 132);
2-(6-(((Cyclopropylmethyl)amino)methyl)-2-methylpyrimidin-4-yl)-6-(4-fluoro-2-(4-methyl-4H-1,2,4-triazol-3-yl)phenyl)isoindolin-1-one (Compound 133);
2-(4-(((Cyclobutylmethyl)amino)methyl)-6-methylpyridin-2-yl)-6-(4-fluoro-2-(4-methyl-4H-1,2,4-triazol-3-yl)phenyl)isoindolin-1-one (Compound 134);
2-(6-Chloro-4-(((cyclobutylmethyl)amino)methyl)pyridin-2-yl)-6-(3-((4-methyl-4H-1,2,4-triazol-3-yl)methyl)oxetan-3-yl)isoindolin-1-one (Compound 144);
2-(6-Chloro-4-(((cyclopropylmethyl)amino)methyl)pyridin-2-yl)-6-(1-((4-methyl-4H-1,2,4-triazol-3-yl)methyl)cyclobutyl)isoindolin-1-one (Compound 145);
2-(4-(((Cyclopropylmethyl)amino)methyl)-6-methylpyridin-2-yl)-6-(3-((4-methyl-4H-1,2,4-triazol-3-yl)methyl)oxetan-3-yl)isoindolin-1-one (Compound 147);
2-(6-Chloro-4-((3-ethylpiperidin-1-yl)methyl)pyridin-2-yl)-6-(3-((4-methyl-4H-1,2,4-triazol-3-yl)methyl)oxetan-3-yl)isoindolin-1-one (Compound 148);
2-(6-Chloro-4-(((3,3,3-trifluoropropyl)amino)methyl)pyridin-2-yl)-6-(1-((4-methyl-4H-1,2,4-triazol-3-yl)methyl)cyclobutyl)isoindolin-1-one (Compound 151);
(S)-2-(6-Chloro-4-((3-methylpiperidin-1-yl)methyl)pyridin-2-yl)-6-(3,3-difluoro-1-((4-methyl-4H-1,2,4-triazol-3-yl)methyl)cyclobutyl)isoindolin-1-one (Compound 152);
3-(2-(6-Chloro-4-(((3,3,3-trifluoropropyl)amino)methyl)pyridin-2-yl)-3-oxoisoindolin-5-yl)-3-((4-methyl-4H-1,2,4-triazol-3-yl)methyl)cyclobutane-1-carbonitrile (Compound 158);
2-(6-Chloro-4-(((cyclopentylmethyl)amino)methyl)pyridin-2-yl)-6-(3-((4-methyl-4H-1,2,4-triazol-3-yl)methyl)oxetan-3-yl)isoindolin-1-one (Compound 159);
(S)-2-(6-Chloro-4-(((cyclopropylmethyl)amino)methyl)pyridin-2-yl)-6-(1-(4-methyl-4H-1,2,4-triazol-3-yl)propan-2-yl)isoindolin-1-one (Compound 160);
(R)-2-(6-Chloro-4-(((cyclopropylmethyl)amino)methyl)pyridin-2-yl)-6-(1-(4-methyl-4H-1,2,4-triazol-3-yl)propan-2-yl)isoindolin-1-one (Compound 161);
2-(4-(((Cyclobutylmethyl)amino)methyl)-6-(trifluoromethyl)pyridin-2-yl)-6-(3-((4-methyl-4H-1,2,4-triazol-3-yl)methyl)oxetan-3-yl)isoindolin-1-one (Compound 162);
(S)-2-(6-Chloro-4-(((3,3,3-trifluoropropyl)amino)methyl)pyridin-2-yl)-6-(1-(4-methyl-4H-1,2,4-triazol-3-yl)propan-2-yl)isoindolin-1-one (Compound 163);
(R)-2-(6-Chloro-4-(1-((cyclopropylmethyl)amino)ethyl)pyridin-2-yl)-6-(3-((4-methyl-4H-1,2,4-triazol-3-yl)methyl)oxetan-3-yl)isoindolin-1-one (Compound 166);
(S)-2-(6-Chloro-4-(1-((cyclopropylmethyl)amino)ethyl)pyridin-2-yl)-6-(3-((4-methyl-4H-1,2,4-triazol-3-yl)methyl)oxetan-3-yl)isoindolin-1-one (Compound 167);
3-(2-(6-Chloro-4-(((cyclopropylmethyl)amino)methyl)pyridin-2-yl)-3-oxoiso-indolin-5-yl)-3-((4-methyl-4H-1,2,4-triazol-3-yl)methyl)cyclobutane-1-carbonitrile (Compound 168);
2-(6-Chloro-4-((((3,3-difluorocyclobutyl)methyl)amino)methyl)pyridin-2-yl)-6-(3-((4-methyl-4H-1,2,4-triazol-3-yl)methyl)oxetan-3-yl)isoindolin-1-one (Compound 169);
(R)-2-(6-Chloro-4-(((cyclobutylmethyl)amino)methyl)pyridin-2-yl)-6-(1-(4-methyl-4H-1,2,4-triazol-3-yl)propan-2-yl)isoindolin-1-one (Compound 170);
(S)-2-(6-Chloro-4-(((cyclobutylmethyl)amino)methyl)pyridin-2-yl)-6-(1-(4-methyl-4H-1,2,4-triazol-3-yl)propan-2-yl)isoindolin-1-one (Compound 171);
2-(6-Chloro-4-((((1-methylcyclobutyl)methyl)amino)methyl)pyridin-2-yl)-6-(3-((4-methyl-4H-1,2,4-triazol-3-yl)methyl)oxetan-3-yl)isoindolin-1-one (Compound 172);
2-(6-Chloro-4-((((2,2-difluorocyclopropyl)methyl)amino)methyl)pyridin-2-yl)-6-(3-((4-methyl-4H-1,2,4-triazol-3-yl)methyl)oxetan-3-yl)isoindolin-1-one (Compound 175);
(1S,3r)-3-(2-(6-Chloro-4-(((S)-3-methylpiperidin-1-yl)methyl)pyridin-2-yl)-3-oxoisoindolin-5-yl)-3-((4-methyl-4H-1,2,4-triazol-3-yl)methyl)cyclobutane-1-carbo-nitrile (Compound 179);
2-(6-Chloro-4-(((S)-3-methylpiperidin-1-yl)methyl)pyridin-2-yl)-6-((S)-1,1,1-trifluoro-3-(4-methyl-4H-1,2,4-triazol-3-yl)propan-2-yl)isoindolin-1-one (Compound 181);
3-(2-(6-Chloro-4-(((cyclobutylmethyl)amino)methyl)pyridin-2-yl)-3-oxoiso-indolin-5-yl)-3-((4-methyl-4H-1,2,4-triazol-3-yl)methyl)cyclobutane-1-carbonitrile (Compound 182);
2-(6-Chloro-4-((((tetrahydrofuran-2-yl)methyl)amino)methyl)pyridin-2-yl)-6-(3-((4-methyl-4H-1,2,4-triazol-3-yl)methyl)oxetan-3-yl)isoindolin-1-one (Compound 188);
2-(6-Chloro-4-(((2-hydroxycyclopentyl)amino)methyl)pyridin-2-yl)-6-(3-((4-methyl-4H-1,2,4-triazol-3-yl)methyl)oxetan-3-yl)isoindolin-1-one (Compound 193);
(S)-2-(6-Chloro-4-((((tetrahydrofuran-2-yl)methyl)amino)methyl)pyridin-2-yl)-6-(3-((4-methyl-4H-1,2,4-triazol-3-yl)methyl)oxetan-3-yl)isoindolin-1-one (Compound 196);
2-(6-Chloro-4-(1-(((2,2-difluorocyclopropyl)methyl)amino)ethyl)pyridin-2-yl)-6-(3-((4-methyl-4H-1,2,4-triazol-3-yl)methyl)oxetan-3-yl)isoindolin-1-one (Compound 199);
2-(6-Chloro-4-((cyclopentylamino)methyl)pyridin-2-yl)-6-(3-((4-methyl-4H-1,2,4-triazol-3-yl)methyl)oxetan-3-yl)isoindolin-1-one (Compound 201);
2-(6-Chloro-4-(((3,3,3-trifluoro-2-methylpropyl)amino)methyl)pyridin-2-yl)-6-(3-((4-methyl-4H-1,2,4-triazol-3-yl)methyl)oxetan-3-yl)isoindolin-1-one (Compound 202);
2-(6-Chloro-4-(1-(((2,2-difluorocyclopropyl)methyl)amino)ethyl)pyridin-2-yl)-6-(3-((4-methyl-4H-1,2,4-triazol-3-yl)methyl)oxetan-3-yl)isoindolin-1-one (Compound 206);
2-(6-Chloro-4-(((2-hydroxycyclopentyl)amino)methyl)pyridin-2-yl)-6-(3-((4-methyl-4H-1,2,4-triazol-3-yl)methyl)oxetan-3-yl)isoindolin-1-one (Compound 209);
2-(6-Chloro-4-(1-((cyclobutylmethyl)amino)ethyl)pyridin-2-yl)-6-(3-((4-methyl-4H-1,2,4-triazol-3-yl)methyl)oxetan-3-yl)isoindolin-1-one (Compound 213);
2-(6-Chloro-4-((R)-1-((((S)-tetrahydrofuran-2-yl)methyl)amino)ethyl)pyridin-2-yl)-6-(3-((4-methyl-4H-1,2,4-triazol-3-yl)methyl)oxetan-3-yl)isoindolin-1-one (Compound 219);
(S)-2-(6-Chloro-4-(1-(((3,3-difluorocyclobutyl)methyl)amino)ethyl)pyridin-2-yl)-6-(3-((4-methyl-4H-1,2,4-triazol-3-yl)methyl)oxetan-3-yl)isoindolin-1-one (Compound 222);
(R)-2-(6-Chloro-4-(((1-cyclopropylethyl)amino)methyl)pyridin-2-yl)-6-(3-((4-methyl-4H-1,2,4-triazol-3-yl)methyl)oxetan-3-yl)isoindolin-1-one (Compound 223);
3-(2-(6-Chloro-4-(1-((cyclopropylmethyl)amino)ethyl)pyridin-2-yl)-3-oxoiso-indolin-5-yl)-3-((4-methyl-4H-1,2,4-triazol-3-yl)methyl)cyclobutane-1-carbonitrile (Compound 225);
3-(2-(6-Chloro-4-((((3,3-difluorocyclobutyl)methyl)amino)methyl)pyridin-2-yl)-3-oxoisoindolin-5-yl)-3-((4-methyl-4H-1,2,4-triazol-3-yl)methyl)cyclobutane-1-carbo-nitrile (Compound 227);
3-(2-(6-Chloro-4-((((2,2-difluorocyclopropyl)methyl)amino)methyl)pyridin-2-yl)-3-oxoisoindolin-5-yl)-3-((4-methyl-4H-1,2,4-triazol-3-yl)methyl)cyclobutane-1-carbonitrile (Compound 230);
(R)-2-(6-Chloro-4-(1-(cyclopentylamino)ethyl)pyridin-2-yl)-6-(3-((4-methyl-4H-1,2,4-triazol-3-yl)methyl)oxetan-3-yl)isoindolin-1-one (Compound 231);
(S)-2-(6-Chloro-4-(1-(cyclopentylamino)ethyl)pyridin-2-yl)-6-(3-((4-methyl-4H-1,2,4-triazol-3-yl)methyl)oxetan-3-yl)isoindolin-1-one (Compound 232);
(1r,3r)-3-(2-(6-Chloro-4-(((cyclopropylmethyl)amino)methyl)pyridin-2-yl)-3-oxoisoindolin-5-yl)-3-((4-methyl-4H-1,2,4-triazol-3-yl)methyl)cyclobutane-1-carbo-nitrile (Compound 233);
(1s,3s)-3-(2-(6-Chloro-4-(((cyclopropylmethyl)amino)methyl)pyridin-2-yl)-3-oxoisoindolin-5-yl)-3-((4-methyl-4H-1,2,4-triazol-3-yl)methyl)cyclobutane-1-carbonitrile (Compound 234);
2-(6-Chloro-4-((R)-1-(((S)-1-cyclopropylethyl)amino)ethyl)pyridin-2-yl)-6-(3-((4-methyl-4H-1,2,4-triazol-3-yl)methyl)oxetan-3-yl)isoindolin-1-one (Compound 239);
(1r,3r)-3-(2-(6-Chloro-4-(((cyclobutylmethyl)amino)methyl)pyridin-2-yl)-3-oxoisoindolin-5-yl)-3-((4-methyl-4H-1,2,4-triazol-3-yl)methyl)cyclobutane-1-carbonitrile (Compound 244);
2-(6-Chloro-4-(1-((2-cyclopropylpropan-2-yl)amino)ethyl)pyridin-2-yl)-6-(3-((4-methyl-4H-1,2,4-triazol-3-yl)methyl)oxetan-3-yl)isoindolin-1-one (Compound 247);
2-(6-Chloro-4-(1-(cyclopentylamino)propyl)pyridin-2-yl)-6-(3-((4-methyl-4H-1,2,4-triazol-3-yl)methyl)oxetan-3-yl)isoindolin-1-one (Compound 251);
2-(6-Chloro-4-(((2-methoxycyclopentyl)amino)methyl)pyridin-2-yl)-6-(3-((4-methyl-4H-1,2,4-triazol-3-yl)methyl)oxetan-3-yl)isoindolin-1-one (Compound 253);
2-(6-Chloro-4-(((cyclopropylmethyl)amino)methyl)pyridin-2-yl)-6-(2-((4-methyl-4H-1,2,4-triazol-3-yl)methyl)spiro[3.3]heptan-2-yl)isoindolin-1-one (Compound 255);
2-(4-(((Cyclopropylmethyl)amino)methyl)-6-methylpyridin-2-yl)-6-(4-fluoro-2-(4-methyl-4H-1,2,4-triazol-3-yl)phenyl)-4-(trifluoromethyl)isoindolin-1-one (Compound 256);
2-(4-((((3,3-Difluorocyclobutyl)methyl)amino)methyl)-6-methylpyridin-2-yl)-6-(4-fluoro-2-(4-methyl-4H-1,2,4-triazol-3-yl)phenyl)isoindolin-1-one (Compound 260);
2-(4-(((Cyclopropylmethyl)amino)methyl)-5-fluoro-6-methylpyridin-2-yl)-6-(4-fluoro-2-(4-methyl-4H-1,2,4-triazol-3-yl)phenyl)isoindolin-1-one (Compound 261);
6-(5-Chloro-3-(4-methyl-4H-1,2,4-triazol-3-yl)pyridin-2-yl)-2-(4-(((cyclo-propylmethyl)amino)methyl)-6-methylpyridin-2-yl)isoindolin-1-one (Compound 263);
2-(4-(((Cyclopropylmethyl)amino)methyl)-6-(trifluoromethyl)pyridin-2-yl)-6-(4-fluoro-2-(4-methyl-4H-1,2,4-triazol-3-yl)phenyl)isoindolin-1-one (Compound 265);
2-(4-(((Cyclopropylmethyl)amino)methyl)-6-methylpyridin-2-yl)-6-(4-methoxy-2-(4-methyl-4H-1,2,4-triazol-3-yl)phenyl)isoindolin-1-one (Compound 273);
6-(4-Chloro-2-(4-methyl-4H-1,2,4-triazol-3-yl)phenyl)-2-(4-(((cyclobutyl-methyl)amino)methyl)-6-methylpyridin-2-yl)isoindolin-1-one (Compound 274);
6-(4-Chloro-2-(4-methyl-4H-1,2,4-triazol-3-yl)phenyl)-2-(6-chloro-4-(2-((cyclopropylmethyl)amino)propan-2-yl)pyridin-2-yl)isoindolin-1-one (Compound 275);
2-(4-(((Cyclopropylmethyl)amino)methyl)-6-methylpyridin-2-yl)-6-(4-methyl-2-(4-methyl-4H-1,2,4-triazol-3-yl)phenyl)isoindolin-1-one (Compound 277);
6-(4-Chloro-2-(4-methyl-4H-1,2,4-triazol-3-yl)phenyl)-2-(4-(1-((cyclobutyl-methyl)amino)ethyl)-6-methylpyridin-2-yl)isoindolin-1-one (Compound 278);
6-(4-Chloro-2-(4-methyl-4H-1,2,4-triazol-3-yl)phenyl)-2-(4-((((3,3-difluoro-cyclobutyl)methyl)amino)methyl)-6-methylpyridin-2-yl)isoindolin-1-one (Compound 279);
6-(4-Chloro-2-(4-methyl-4H-1,2,4-triazol-3-yl)phenyl)-2-(3-(((cyclopropyl-methyl)amino)methyl)-5-methylphenyl)isoindolin-1-one (Compound 280);
6-(4-Chloro-2-(4-methyl-4H-1,2,4-triazol-3-yl)phenyl)-2-(4-(1-(((3,3-difluoro-cyclobutyl)methyl)amino)ethyl)-6-methylpyridin-2-yl)isoindolin-1-one (Compound 281);
2-(4-(1-((Cyclopropylmethyl)amino)ethyl)-6-methylpyridin-2-yl)-6-(4-fluoro-2-(4-methyl-4H-1,2,4-triazol-3-yl)phenyl)isoindolin-1-one (Compound 282);
2-(4-(((Cyclopropylmethyl)amino)methyl)-6-methylpyridin-2-yl)-6-(4-fluoro-2-(4-methylisoxazol-3-yl)phenyl)isoindolin-1-one (Compound 289);
2-(4-(2-((Cyclopropylmethyl)amino)propan-2-yl)-6-methylpyridin-2-yl)-6-(4-fluoro-2-(4-methyl-4H-1,2,4-triazol-3-yl)phenyl)isoindolin-1-one (Compound 293);
2-(4-(((Cyclopropylmethyl)amino)methyl)-6-methylpyridin-2-yl)-6-(2-(4-ethyl-4H-1,2,4-triazol-3-yl)-4-fluorophenyl)isoindolin-1-one (Compound 294);
2-(4-((sec-Butylamino)methyl)-6-methylpyridin-2-yl)-6-(4-fluoro-2-(4-methyl-4H-1,2,4-triazol-3-yl)phenyl)isoindolin-1-one (Compound 295);
2-(4-(((Dicyclopropylmethyl)amino)methyl)-6-methylpyridin-2-yl)-6-(4-fluoro-2-(4-methyl-4H-1,2,4-triazol-3-yl)phenyl)isoindolin-1-one (Compound 298);
6-(4-Fluoro-2-(4-methyl-4H-1,2,4-triazol-3-yl)phenyl)-2-(4-((isopentylamino)-methyl)-6-methylpyridin-2-yl)isoindolin-1-one (Compound 300);
6-(4-Fluoro-2-(4-methyl-4H-1,2,4-triazol-3-yl)phenyl)-2-(4-(((2-methoxyethyl)-amino)methyl)-6-methylpyridin-2-yl)isoindolin-1-one (Compound 302);
6-(4-Fluoro-2-(4-methyl-4H-1,2,4-triazol-3-yl)phenyl)-2-(4-((isobutylamino)-methyl)-6-methylpyridin-2-yl)isoindolin-1-one (Compound 303);
2-(4-(((2-Cyclopropylethyl)amino)methyl)-6-methylpyridin-2-yl)-6-(4-fluoro-2-(4-methyl-4H-1,2,4-triazol-3-yl)phenyl)isoindolin-1-one (Compound 307);
6-(4-Fluoro-2-(4-methyl-4H-1,2,4-triazol-3-yl)phenyl)-2-(4-(((2-hydroxy-2-methylpropyl)amino)methyl)-6-methylpyridin-2-yl)isoindolin-1-one (Compound 309);
2-(4-(((Cyclopropylmethyl)amino)methyl)-6-methylpyridin-2-yl)-6-(4-fluoro-2-(4-(methyl-d3)-4H-1,2,4-triazol-3-yl)phenyl)isoindolin-1-one (Compound 310);
6-(4-Fluoro-2-(4-methyl-4H-1,2,4-triazol-3-yl)phenyl)-2-(4-((((1R,2R)-2-hydroxycyclopentyl)amino)methyl)-6-methylpyridin-2-yl)isoindolin-1-one (Compound 314);
(R)-6-(4-Fluoro-2-(4-methyl-4H-1,2,4-triazol-3-yl)phenyl)-2-(4-(((1-hydroxy-3-methylbutan-2-yl)amino)methyl)-6-methylpyridin-2-yl)isoindolin-1-one (Compound 315);
6-(4-Fluoro-2-(4-methyl-4H-1,2,4-triazol-3-yl)phenyl)-2-(4-((((1-hydroxy-cyclopentyl)methyl)amino)methyl)-6-methylpyridin-2-yl)isoindolin-1-one (Compound 319);
6-(2-(4-Cyclopropyl-4H-1,2,4-triazol-3-yl)-4-fluorophenyl)-2-(4-(((cyclopropyl-methyl)amino)methyl)-6-methylpyridin-2-yl)isoindolin-1-one (Compound 320);
6-(4-Fluoro-2-(4-methyl-4H-1,2,4-triazol-3-yl)phenyl)-2-(6-methyl-4-((propyl-amino)methyl)pyridin-2-yl)isoindolin-1-one (Compound 323);
6-(4-Fluoro-2-(4-methyl-4H-1,2,4-triazol-3-yl)phenyl)-2-(6-methyl-4-(((oxetan-2-ylmethyl)amino)methyl)pyridin-2-yl)isoindolin-1-one (Compound 325);
6-(4-Fluoro-2-(4-methyl-4H-1,2,4-triazol-3-yl)phenyl)-2-(6-methyl-4-(((3,3,3-trifluoropropyl)amino)methyl)pyridin-2-yl)isoindolin-1-one (Compound 326);
6-(4-Fluoro-2-(4-methyl-4H-1,2,4-triazol-3-yl)phenyl)-2-(6-methyl-4-(((4,4,4-trifluorobutyl)amino)methyl)pyridin-2-yl)isoindolin-1-one (Compound 328);
2-(4-(((Cyclopentylmethyl)amino)methyl)-6-methylpyridin-2-yl)-6-(4-fluoro-2-(4-methyl-4H-1,2,4-triazol-3-yl)phenyl)isoindolin-1-one (Compound 329);
2-(4-(((Cyclopropylmethyl)amino)methyl)-6-methyl-5-(trifluoromethyl)pyridin-2-yl)-6-(4-fluoro-2-(4-methyl-4H-1,2,4-triazol-3-yl)phenyl)isoindolin-1-one (Compound 337);
   and tautomers and pharmaceutically acceptable salts thereof.

Compounds of the invention may be administered to a patient in therapeutically effective amounts which range usually from about 0.5 to about 2000 mg, more typically form about 1 to about 500 mg, for example from about 2 to about 100 mg, daily depending on the age, sex, weight, condition of the patient, condition to be treated, administration route and the active ingredient used. The compounds of the invention can be formulated into dosage forms using the principles known in the art. The compound can be given to a patient as such or in combination with suitable pharmaceutical excipients in the form of tablets, granules, capsules, suppositories, emulsions, suspensions or solutions. Suitable carriers, solvents, gel forming ingredients, dispersion forming ingredients, antioxidants, colours, sweeteners, wetting compounds and other ingredients normally used in this field of technology may also be used. The compositions containing the active compound can be given enterally or parenterally, the oral route being the preferred way. The contents of the active compound in the composition is from about 0.5 to 100%, typically from about 0.5 to about 20%, per weight of the total composition.

The compounds of the invention can be given to the subject as the sole active ingredient or in combination with one of more other active ingredients for treatment of a particular disease.

In the treatment of diseases and conditions wherein inhibition of Cbl-b is desired, such as various cancers, a combination of therapeutic agents and/or other treatments (e.g., radiation therapy) is often advantageous. The second (or third) agent to be administered may have the same or different mechanism of action than the primary therapeutic agent.

Accordingly, a compound of the invention may be administered in combination with other anti-cancer treatments useful in the treatment of cancers. For example, a compound of the invention can be packaged together with instructions that the compound is to be used in combination with other anti-cancer agents and treatments for the treatment of cancer. The present invention further comprises combinations of a compound of the invention and one or more additional agents in kit form, for example, where they are packaged together or placed in separate packages to be sold together as a kit, or where they are packaged to be formulated together.

According to one embodiment of the invention, the therapeutically effective amount of a compound of formula (I) or a pharmaceutically acceptable salt thereof is co-administered with one or more anti-cancer agents or pain reliever agents.

The optional other anti-cancer agents which can be administered in addition to a compound of formula (I) or a pharmaceutically acceptable salt thereof include, but are not limited to,
chemotherapeutic agents (e.g. docetaxel and paclitaxel),
tyrosine kinase inhibitors including EGFR inhibitors (e.g. gefitinib and osimertinib), VEGFR inhibitors (e.g. bevacizumab) and FGFR inhibitors (e.g. erdafitinib);
immune checkpoint inhibitors (e.g. PD-1, PD-L1 or CTLA-4 antagonists such as nivolumab and pembrolizumab),
epigenetic modulators (e.g. BET inhibitors and HDAC inhibitors),
mTOR inhibitors (e.g. everolimus);
AKT inhibitors (e.g. ipatasertib);
radiopharmaceuticals (e.g. alpharadin);
GnRH/LHRH analogues (such as leuprorelin);
PI3K inhibitors (e.g. idelalisib); and
CDK4/6 inhibitors (e.g. ribocyclib)
steroidogenesis inhibitors (e.g. CYP17A1 inhibitors such as abiraterone acetate and seviteronel); and
a non-steroidal androgen receptor antagonist (e.g. enzalutamide, apalutamide and darolutamide).

The above other therapeutic agents, when employed in combination with a compound of the invention can be used, for example, in those amounts indicated in the Physicians' Desk Reference (PDR) or as otherwise determined by one of ordinary skill in the art.

The compounds of the invention can be prepared by a variety of synthetic routes analogously to the methods known in the literature using suitable starting materials. The present invention will be explained in more detail by the following experiments and examples. The experiments and examples are meant only for illustrating purposes and do not limit the scope of the invention defined in claims.

### EXAMPLES

### Intermediate 1. 6-(3-((4-Methyl-4H-1,2,4-triazol-3-yl) methyl) oxetan-3-yl) isoindolin-1-one

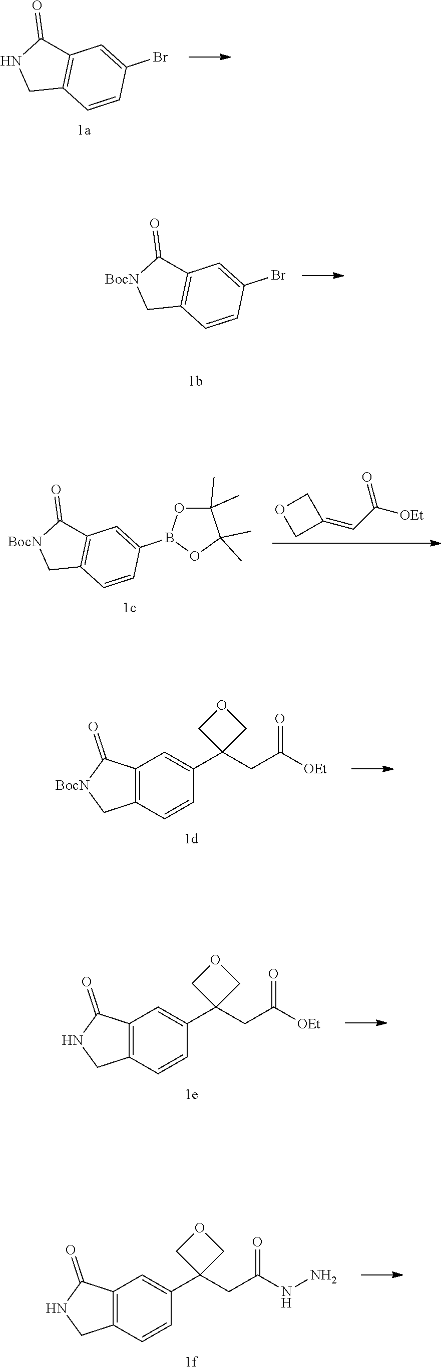

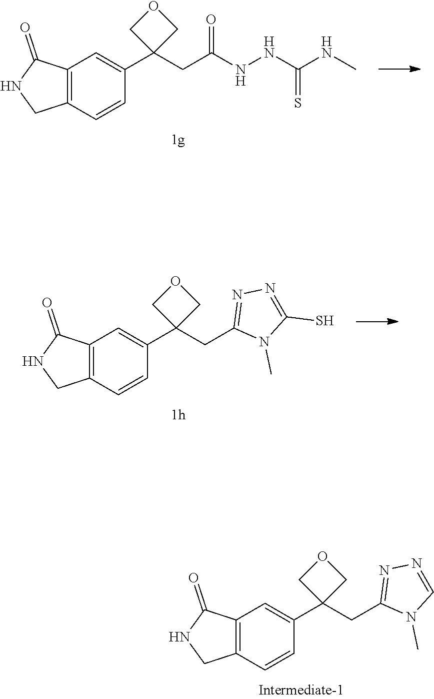 [SMILES: CNC(=S)NNC(=O)CC1(c2ccc3c(c2)C(=O)NC3)COC1 ; Cn1c(S)nnc1CC1(c2ccc3c(c2)C(=O)NC3)COC1 ; Cn1cnnc1CC1(c2ccc3c(c2)C(=O)NC3)COC1]

### a) tert-Butyl 6-bromo-1-oxoisoindoline-2-carboxylate (1b)

To a stirred solution of 6-bromoisoindolin-1-one (70.0 g, 330.11 mmol) and 4-dimethylaminopyridine (4.066 g, 33.01 mmol) in THF (700.0 mL) was added di-tert-butyl di-carbonate (180.12 g, 825.27 mmol) dropwise at 0° C. The mixture was stirred at RT for 1 h. Then the reaction mixture was quenched with saturated aqueous ammonium chloride and extracted with ethyl acetate. The combined organic layer was dried over anhydrous sodium sulphate, filtered, and concentrated. The crude mixture was dissolved with DCM and triturated with pentane to obtain precipitate. The precipitate was filtered, dried over vacuum to afford the title compound (84.0 g, 81.52%). ¹H NMR (400 MHz, CDCl₃) δ: 8.02 (d, 1H), 7.74 (dd, 1H), 7.36 (d, 1H), 4.70 (s, 2H), 1.62 (s, 9H).

### b) tert-Butyl 1-oxo-6-(4,4,5,5-tetramethyl-1,3,2-dioxaborolan-2-yl) isoindoline-2-carboxylate (1c)

To a degassed solution of tert-butyl 6-bromo-1-oxoisoindoline-2-carboxylate (30.0 g, 96.11 mmol), 4,4,5,5-tetramethyl-2-(4,4,5,5-tetramethyl-1,3,2-dioxaborolan-2-yl)-1,3,2-dioxaborolane (29.286 g, 115.32 mmol), and potassium acetate (12.26 g, 124.9 mmol) in 1,4-dioxane (300.0 mL) was added [1,1′-bis(diphenylphosphino)ferrocene]-dichloropalladium(II) complex with DCM (3.921 g, 4.8 mmol) followed by stirring the mixture at 80° C. for 2 h. Then the reaction mixture was cooled to RT, filtered through celite pad, followed by washing the celite pad with ethyl acetate. The filtrate was concentrated, diluted with water, and extracted with 40% ethyl acetate in hexane. The combined organic layers were dried over anhydrous sodium sulphate, filtered, and concentrated. The obtained residue was dissolved in DCM and triturated with mixture of methyl tert-butyl ether (MTBE) and pentane to obtain precipitate. The filtered precipitate was dried under vacuum to afford the title compound (23.0 g, 66.62%). ¹H NMR (400 MHz, CDCl₃) δ: 8.39 (s, 1H), 8.06 (dd, 1H), 7.48 (dd, 1H), 4.78 (s, 2H), 1.63 (s, 9H), 1.37 (s, 12H).

### c) tert-Butyl 6-(3-(2-ethoxy-2-oxoethyl) oxetan-3-yl)-1-oxoisoindoline-2-carboxylate (1d)

To a degassed solution of tert-butyl 1-oxo-6-(4,4,5,5-tetramethyl-1,3,2-dioxa-borolan-2-yl) isoindoline-2-carboxylate (45.0 g, 125.26 mmol), ethyl 2-(oxetan-3-yl-idene) acetate (26.7 g, 187.9 mmol) and N, N-diethylethanamine (25.35 g, 250.05 mmol) in 1,4-dioxane (450.0 mL) and water (400.0 mL) was added [Rh(COD)Cl]₂ (3.09 g, 6.26 mmol) followed by stirring the mixture for 1 h at RT. The reaction mixture was quenched with water and extracted with ethyl acetate. The combined organic layer was dried over anhydrous sodium sulphate, filtered, concentrated, and purified by silica-gel flash column chromatography using 20-40% ethyl acetate in hexane as eluent to afford the title compound (29.0 g, 61.67%). ¹H NMR (400 MHz, CDCl₃) δ: 7.66 (s, 1H), 7.52-7.45 (m, 2H), 4.99 (d, 2H), 4.87 (d, 2H), 4.73 (s, 2H), 4.02-3.96 (m, 2H), 3.15 (s, 2H), 1.60 (s, 9H), 1.12 (t, 3H).

### d) Ethyl 2-(3-(3-oxoisoindolin-5-yl) oxetan-3-yl) acetate (1e)

To a solution of tert-butyl 6-(3-(2-ethoxy-2-oxoethyl) oxetan-3-yl)-1-oxoiso-indoline-2-carboxylate (13.5 g, 35.96 mmol) in 1,1,1,3,3,3-hexafluoro-2-propanol (135.0 mL) was added HCl (5.40 mL, 4 M in dioxane) at 0° C. Then the mixture was slowly warmed to RT and stirred for 4 h. Then the mixture was concentrated and purified by silica-gel flash column chromatography using 0-2.5% methanol in dichloromethane as eluent to afford the title compound as off white solid (6.0 g, 60.61%). LCMS: 276.1 [M+H]⁺.

### e) 2-(3-(3-Oxoisoindolin-5-yl) oxetan-3-yl) aceto hydrazide (1f)

To a solution of ethyl 2-(3-(3-oxoisoindolin-5-yl) oxetan-3-yl) acetate (14.0 g, 50.85 mmol) in ethanol (70.0 mL) was added hydrazine hydrate (38.18 g, 762.8 mmol) followed by stirring the mixture at 75° C. for 24 h. The mixture was concentrated and triturated with 5% methanol in dichloromethane to afford the title compound as off-white solid (8.9 g, 66.98%). LCMS: 262.1 [M+H]⁺.

### f) N-Methyl-2-(2-(3-(3-oxoisoindolin-5-yl) oxetan-3-yl) acetyl) hydrazine-1-carbothioamide (1g)

To a solution of 2-(3-(3-oxoisoindolin-5-yl) oxetan-3-yl) aceto hydrazide (12.50 g, 47.84 mmol) in THF (120.0 mL) was added methyl isothiocyanate (8.74 g, 119.59 mmol) followed by stirring at 70° C. for 6 h. The precipitated solid was filtered, washed with cold THF and dried under vacuum to afford the title compound as off-white solid (14.0 g, 87.51%). LCMS: 335.1 [M+H]⁺.
g) 6-(3-((5-Mercapto-4-methyl-4H-1,2,4-triazol-3-yl) methyl) oxetan-3-yl) isoindolin-1-one (1h)

N-methyl-2-(2-(3-(3-oxoisoindolin-5-yl) oxetan-3-yl) acetyl) hydrazine-1-carbothioamide (5.8 g, 17.344 mmol) in 1 N NaOH (29.0 mL) was stirred at RT for 4 h. Then the reaction mixture was quenched with ice water and pH was adjusted to about 4 with 1 N HCl. The precipitate was filtered and dried under vacuum to afford the title compound as off-white solid (4.70 g, 85.65%), LCMS: 317.1 [M+H]⁺.

### h) 6-(3-((4-Methyl-4H-1,2,4-triazol-3-yl) methyl) oxetan-3-yl) isoindolin-1-one (Intermediate-1)

To a solution of 6-(3-((5-mercapto-4-methyl-4H-1,2,4-triazol-3-yl) methyl) oxetan-3-yl) isoindolin-1-one (4.70 g, 14.86 mmol) and acetic acid (7.8 mL) in dichloromethane (32.0 mL) was added hydrogen peroxide (6.6 mL) at 0° C. The mixture was slowly warmed to RT and stirred for 1 h. The mixture was concentrated and co-evaporated with toluene twice. The obtained semisolid was triturated with methanol, filtered, and dried under reduced pressure to afford the title compound as an off-white solid (3.5 g, 82.87%), LCMS: 285.1 [M+H]⁺.

The following intermediates were prepared according to the procedure described for Intermediate-1 from the starting materials indicated in the Table with appropriate variations in reactants, quantities of reagents, coupling methods and solvents. The characterization data of the compounds are also summarized in the Table.

| | | LCMS | |
|---|---|---|---|
| No. | Structure | [M + H]⁺ | Starting material |
| Int-2 | 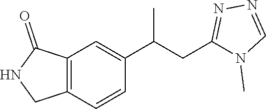 [SMILES: CC(Cc1nncn1C)c1ccc2c(c1)C(=O)NC2] | 257.1 | 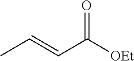 |
| Int-3 | 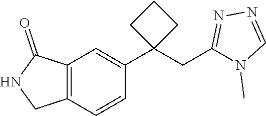 [SMILES: Cn1cnnc1CC1(c2ccc3c(c2)C(=O)NC3)CCC1] | 283.1 | 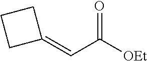 |

### Intermediate-4. (S)-1-(3-Bromo-5-(trifluoromethyl)benzyl)-3-methylpiperidine (Procedure A)

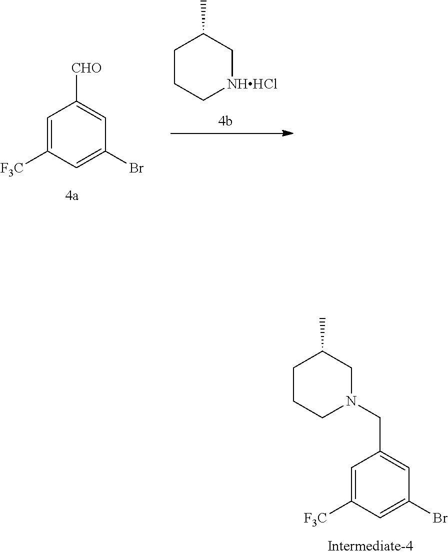

To a solution of (S)-3-methylpiperidine hydrochloride (0.67 g, 4.94 mmol) in DCM (10 mL) was added DIPEA (1.70 mL, 9.75 mmol) followed by stirring for 30 min. To this mixture was added 3-bromo-5-(trifluoromethyl)benzaldehyde (0.50 g, 1.98 mmol) followed by sodium triacetoxyborohydride (STAB) (1.69 g, 7.99 mmol) at 0° C. The mixture was then stirred at RT for 12 h. The reaction mixture was quenched with water and extracted with DCM. The combined organic layer was dried over anhydrous sodium sulphate, filtered, and concentrated. The obtained crude product was purified by silica-gel flash column chromatography using 0-30% ethyl acetate in hexane as eluent to afford the title compound (0.6 g, 90%). LCMS: 338.0 [M+2+H]⁺.

### Intermediate 5. N-(3-Bromo-5-(methyl)benzyl)-1-cyclopropylmethanamine (Procedure B)

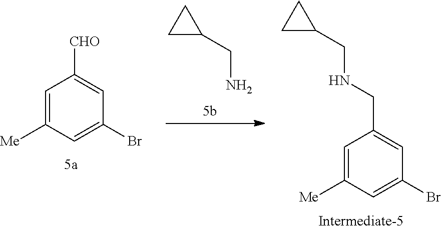 [SMILES: Cc1cc(Br)cc(C=O)c1 ; NCC1CC1 ; Cc1cc(Br)cc(CNCC2CC2)c1]

To a solution of cyclopropylmethanamine (0.36 g, 5.02 mmol) and 3-bromo-5-methylbenzaldehyde (0.40 g, 2.01 mmol) in DCM (10 mL) was added a catalytic amount of acetic acid followed by stirring for 30 min. STAB (2.56 g, 12.06 mmol) was added at 0° C. and the mixture was stirred at RT for 12 h. The reaction mixture was quenched with water and extracted with DCM. The combined organic layer was dried over anhydrous sodium sulphate, filtered, and concentrated. The crude product was purified by silica-gel flash column chromatography using 3-5% MeOH in DCM as eluent to afford the title compound (0.35 g, 68%). LCMS: 256.0 [M+2+H]⁺.

### Intermediate 6. (S)-1-(3-Bromo-5-(piperidin-1-yl)benzyl)-3-methylpiperidine

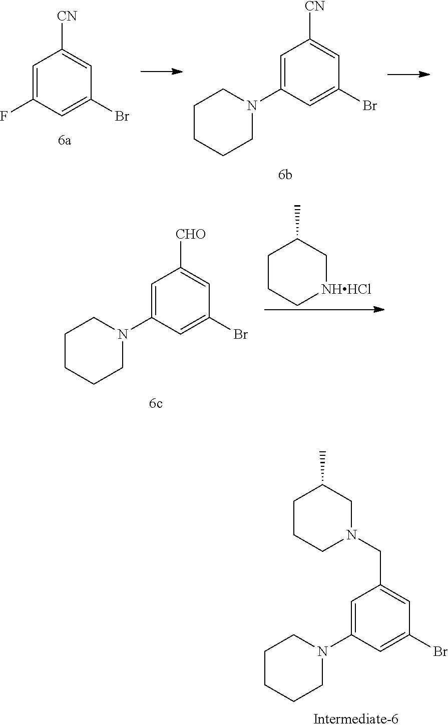

### a) 3-Bromo-5-(piperidin-1-yl) benzonitrile (6b)

A mixture of 3-bromo-5-fluorobenzonitrile (0.5 g, 2.5 mmol), piperidine (0.63 g, 7.5 mmol) and K₂CO₃ (1.0 g, 7.50 mmol) in DMSO (2 mL) was stirred at 120° C. for 16 h. The mixture was cooled to RT, quenched with water and extracted with ethyl acetate. The combined organic layer was dried over anhydrous sodium sulphate, filtered and concentrated. The crude product was purified by silica-gel flash column chromatography using 10% ethyl acetate in hexane as eluent to afford the title compound (0.6 g, 90.51%). LCMS: 267.0 [M+H]⁺.

### b) 3-Bromo-5-(piperidin-1-yl) benzaldehyde (6c)

To a solution of 3-bromo-5-(piperidin-1-yl) benzonitrile (0.47 g, 1.80 mmol) in DCM (5 mL) was added diisobutylaluminium hydride (DIBAL-H) (2.7 mL, 2.7 mmol, 1 M in THF) at 0° C. followed by stirring at RT for 4 h. The reaction mixture was quenched with water and extracted with DCM. The combined organic layer was dried over anhydrous sodium sulphate, filtered, concentrated. The obtained crude product was purified by silica-gel flash column chromatography using 10% ethyl acetate in hexane as eluent to afford the title compound (0.28 g, 58.36%). LCMS: 268.0 [M+H]⁺.

### c) (S)-1-(3-bromo-5-(piperidin-1-yl)benzyl)-3-methylpiperidine (Intermediate-6)

The compound was prepared according to the procedure described for Intermediate-2 with appropriate variations in reactants, quantities of reagents, and solvents. LCMS: 353.1 [M+2+H]⁺.

The following intermediates were prepared according to the procedure described for Intermediate-6 from the starting materials indicated in the Table with appropriate variations in reactants, quantities of reagents, coupling methods and solvents. The characterization data of the compounds are also summarized in the Table.

| | | LCMS | Starting |
|---|---|---|---|
| No. | Structure | [M + H]⁺ | material |
| Int-7 | 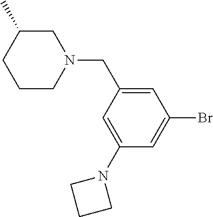 [SMILES: C[C@H]1CCCN(Cc2cc(Br)cc(N3CCC3)c2)C1] | 323.0 | 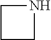 |
| Int-8 | 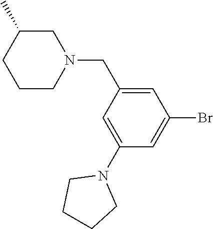 [SMILES: C[C@H]1CCCN(Cc2cc(Br)cc(N3CCCC3)c2)C1] | 339.1 | 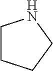 |

### Intermediate 9. (S)-2-Chloro-4-((3-methylpiperidin-1-yl)methyl)-6-(trifluoro-methyl)pyridine

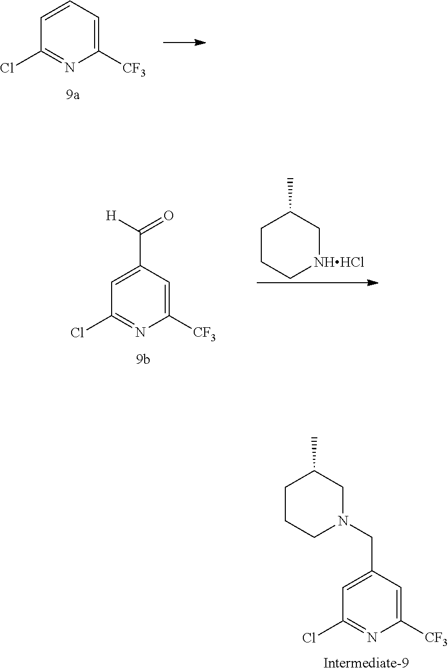

### a) 2-Chloro-6-(trifluoromethyl)isonicotinaldehyde (9b)

To a solution of 2-chloro-6-(trifluoromethyl) pyridine (5.0 g, 27.42 mmol) in dry THF (100 mL) was added 2,2,6,6-Tetramethyl piperidinyl magnesium chloride lithium chloride complex (13.3 g, 55.080 mmol) dropwise at 0° C. followed by stirring at RT for 1 h. Then the mixture was cooled to −78° C. and dry DMF (4.2 mL in 25 mL THF) was added. The mixture was slowly warmed to RT and stirred for 16 h. The mixture was cooled to 0° C., quenched with aqueous ammonium chloride solution and extracted with ethyl acetate. The combined ethyl acetate layer was dried over anhydrous sodium sulphate, filtered, concentrated. The crude product was purified by silica-gel flash column chromatography using 0-10% ethyl acetate in hexane as eluent to afford the title compound as a liquid (0.9 g, 15%). ¹H NMR (CDCl₃, 400 MHz) δ: 10.11 (s, 1H), 8.03 (s, 1H), 7.95 (s, 1H).

### b) (S)-2-Chloro-4-((3-methylpiperidin-1-yl)methyl)-6-(trifluoromethyl)pyridine (Intermediate-9)

The compound was prepared according to the procedure described for Intermediate-4 with appropriate variations in reactants, quantities of reagents, and solvents. LCMS: 293.0 [M+H]⁺.

### Intermediates 10 and 11. (S)-1-(3-Bromo-5-vinylbenzyl)-3-methylpiperidine (Intermediate-10) and (S)-1-(3-Bromo-5-(cyclopropyl methyl) benzyl)-3-methyl-piperidine (Intermediate-11)

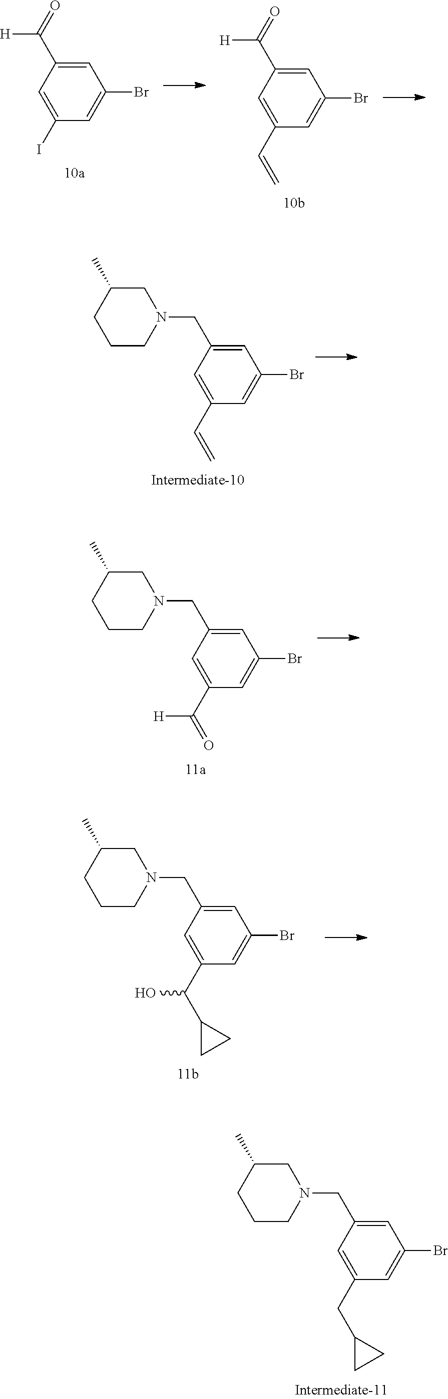 [SMILES: O=Cc1cc(Br)cc(I)c1 ; C=Cc1cc(Br)cc(C=O)c1 ; C=Cc1cc(Br)cc(CN2CCC[C@H](C)C2)c1 ; C[C@H]1CCCN(Cc2cc(Br)cc(C=O)c2)C1 ; C[C@H]1CCCN(Cc2cc(Br)cc(C(O)C3CC3)c2)C1 ; C[C@H]1CCCN(Cc2cc(Br)cc(CC3CC3)c2)C1]

### a) 3-Bromo-5-vinylbenzaldehyde (10b)

To a degassed solution of 3-bromo-5-iodobenzaldehyde (2.70 g, 8.68 mmol), potassium trifluoro(vinyl)borate (1.40 g, 10.42 mmol), and sodium carbonate (1.38 g, 13.02 mmol) in 1,4-dioxane (30.0 mL) and water (18.0 mL) was added bis(triphenyl-phosphine)palladium (II) dichloride (0.61 g, 0.86 mmol) followed by stirring at 70° C. for 2 h. The mixture was cooled to RT, filtered through celite pad, followed by washing the celite pad with ethyl acetate. The filtrate was concentrated, diluted with water, and extracted with ethyl acetate. The combined organic layer was washed with brine, dried over anhydrous sodium sulphate, filtered, and concentrated. The crude product was purified by silica-gel flash column chromatography using 8-10% ethyl acetate in hexane as eluent to afford the title compound as a colourless liquid (1.50 g, 81%). ¹H NMR (400 MHz, CDCl₃) δ: 10.00 (s, 1H), 7.90 (dd, 1H), 7.84 (dd, 1H), 7.80 (dd, 1H), 6.77-6.69 (m, −1H), 5.89 (d, 1H), 5.45 (d, 1H).

### b) (S)-1-(3-Bromo-5-vinylbenzyl)-3-methylpiperidine (Intermediate-10)

The compound was prepared according to the procedure described for Intermediate-4 with appropriate variations in reactants, quantities of reagents, and solvents. LCMS: 294.1 [M+H]⁺.

### c) (S)-3-Bromo-5-((3-methylpiperidin-1-yl) methyl) benzaldehyde (11a)

To a solution of (S)-1-(3-bromo-5-vinylbenzyl)-3-methylpiperidine (1.50 g, 5.10 mmol) and sodium periodate (2.18 g, 10.19 mmol) in THF (20.0 mL) and water (10.0 mL) was added osmium tetroxide (0.97 mL, 0.15 mmol, 4 wt-% in H₂O) dropwise at 0° C. The mixture was slowly warmed to RT and stirred for 4 h. The reaction mixture was quenched with ice water and extracted with ethyl acetate. The combined organic layer was washed with brine, dried over anhydrous sodium sulphate, filtered, and concentrated. The crude product was purified by silica-gel flash column chromatography using 8-9% ethyl acetate in hexane as eluent to afford the title compound as a liquid (0.90 g, 59%). LCMS: 295.95 [M+H]⁺.

### d) (3-Bromo-5-(((S)-3-methylpiperidin-1-yl) methyl)phenyl)(cyclopropyl)-methanol (11 b)

To a solution of (S)-3-bromo-5-((3-methylpiperidin-1-yl) methyl)benzaldehyde (1.20 g, 4.05 mmol) in THF (4.0 mL) was added cyclopropyl magnesium bromide solution (19.50 mL, 9.72 mmol, 0.5 M in THF) at 0° C. Then mixture was slowly warmed to RT and stirred for 16 h. The reaction mixture was quenched with saturated aqueous NH₄Cl solution and extracted with ethyl acetate. The combined organic layer was washed brine, dried over anhydrous sodium sulphate, filtered, and concentrated. The crude product was purified by silica-gel flash column chromatography using 20-25% ethyl acetate in hexane as eluent to afford the title compound as a liquid (0.60 g, 43%). LCMS: 338.10 [M+H]⁺.

### e) (S)-1-(3-Bromo-5-(cyclopropyl methyl) benzyl)-3-methylpiperidine (Intermediate-11)

To a solution of (3-bromo-5-(((S)-3-methylpiperidin-1-yl) methyl) phenyl) (cyclopropyl)methanol (0.45 g, 1.33 mmol) in dichloroethane was added trifluoroacetic acid (0.15 g, 1.35 mmol) and triethyl silane (0.16 g, 1.35 mmol) at 0° C. The resulting mixture was slowly warmed to RT and then stirred at 70° C. for 16 h. The mixture was then concentrated. The crude product was purified by reverse phase HPLC [mobile phase: 0-80% of 0.05% TFA in water and MeCN, column: LUNA OMEG PS C-18 (250×21.2 mm) 5μ] to afford the title compound as colorless liquid (0.20 g, 46%). LCMS: 324.10 [M+H]⁺.

### Intermediate 12. (S)-1-(3-Bromo-5-ethylbenzyl)-3-methylpiperidine

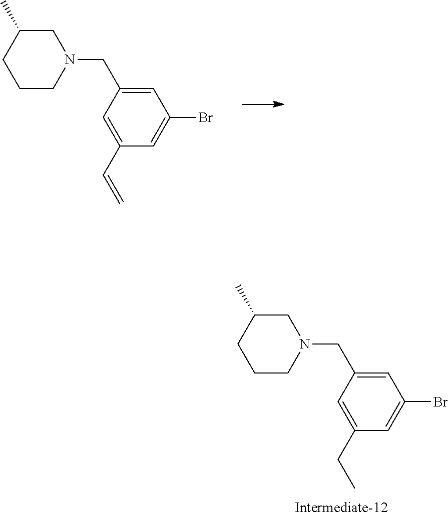 [SMILES: C=Cc1cc(Br)cc(CN2CCC[C@H](C)C2)c1 ; CCc1cc(Br)cc(CN2CCC[C@H](C)C2)c1]

To a solution of (S)-1-(3-bromo-5-vinylbenzyl)-3-methylpiperidine (0.22 g, 0.75 mmol) and sodium acetate trihydrate (0.51 g, 3.74 mmol) in THF was added p-toluene sulfonyl hydrazide (0.70 g, 3.74 mmol) followed by stirring at 75° C. for 12 h. The mixture was filtered and washed with diethyl ether:hexane (1:2). The filtrate was concentrated. The crude product was purified by silica-gel flash column chromatography using 6% ethyl acetate in hexane as eluent to afford the title compound as colorless liquid (0.17 g, 76%). LCMS: 296.10 [M+H]⁺.

### Intermediate-13. (S)-4-Chloro-2-methyl-6-((3-methylpiperidin-1-yl)methyl)-pyrimidine

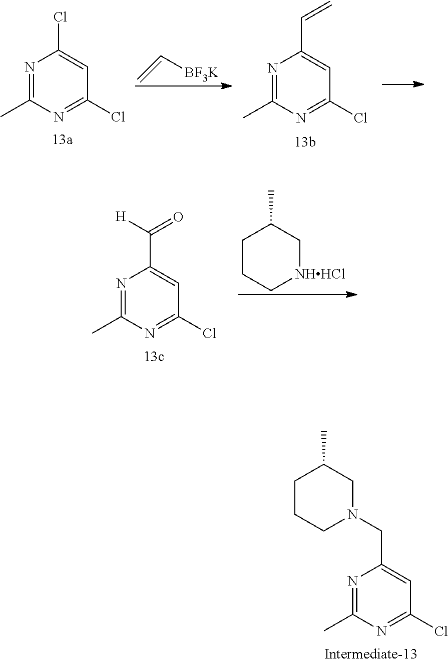

### a) 4-Chloro-2-methyl-6-vinylpyrimidine (13b)

The compound was prepared according to the procedure described for Intermediate-10b with appropriate variations in reactants, quantities of reagents, and solvents. LCMS: 155.10 [M+H]⁺.

### b) 6-Chloro-2-methylpyrimidine-4-carbaldehyde (13c)

The compound was prepared according to the procedure described for Intermediate-11 a with appropriate variations in reactants, quantities of reagents, and solvents. LCMS: 157.10 [M+H]⁺.

### c) (S)-4-Chloro-2-methyl-6-((3-methylpiperidin-1-yl)methyl)pyrimidine (Intermediate-13)

The compound was prepared according to the procedure described for Intermediate-4 with appropriate variations in reactants, quantities of reagents, and solvents. LCMS: 240.1 [M+H]⁺.

### Intermediate 14. (S)-1-(3-Bromo-5-cyclopropylbenzyl)-3-methylpiperidine

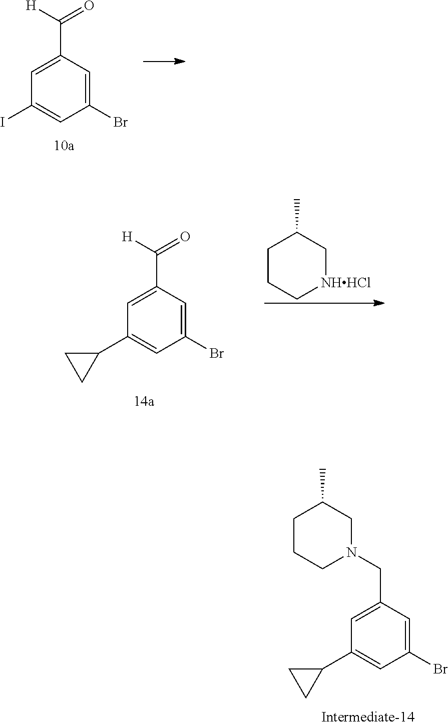

### a) 3-Bromo-5-cyclopropylbenzaldehyde (14a)

The compound was prepared according to the procedure described for Intermediate-11 with appropriate variations in reactants, quantities of reagents, and solvents. ¹H NMR (400 MHz, CDCl₃) δ: 9.93 (s, 1H), 7.99-7.98 (m, 1H), 7.51-7.48 (m, 1H), 2.00-1.93 (m, 1H), 1.11-1.06 (m, 2H), 0.81-0.77 (m, 2H).

### b) (S)-1-(3-Bromo-5-cyclopropylbenzyl)-3-methylpiperidine (Intermediate-14)

The compound was prepared according to the procedure described for Intermediate-4 with appropriate variations in reactants, quantities of reagents, and solvents. LCMS: 310.1 [M+2+H]⁺.

### Intermediate-15. (S)-4-Bromo-2-methyl-6-((3-methylpiperidin-1-yl)methyl)-pyridine

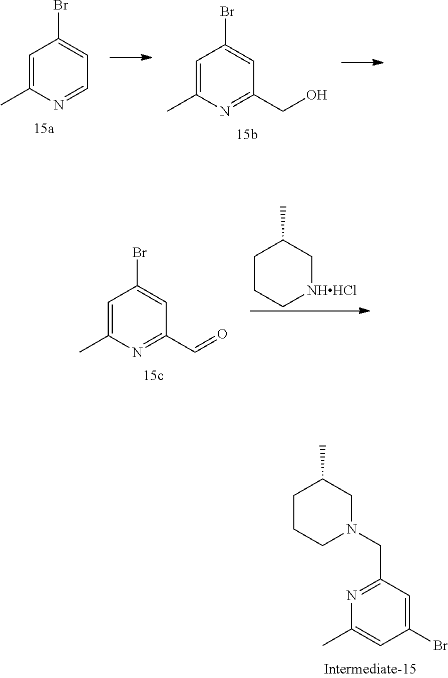

### a) (4-Bromo-6-methylpyridin-2-yl) methanol (15b)

To a solution 4-bromo-2-methylpyridine (3.0 g, 17.44 mmol) and sulfuric acid (0.15 mL) in methanol (75.0 mL) was added ammonium peroxydisulfate (3.979 g, 17.430 mmol) in water (6.6 mL) followed by stirring at 70° C. for 2 h. The mixture was concentrated, diluted with aqueous sodium bicarbonate solution, and extracted with ethyl acetate. The combined organic layer was dried over anhydrous sodium sulphate, filtered and concentrated. The crude product was purified by silica-gel flash column chromatography using 0-50% ethyl acetate in hexane as eluent to afford the title compound (0.950 g, 26.96%). LCMS: 203.9 [M+H]⁺.

### b) 4-Bromo-6-methylpicolinaldehyde (15c)

To a solution (4-bromo-6-methylpyridin-2-yl) methanol (0.90 g, 4.45 mmol) in dichloromethane (10.0 mL) was added Dess-Martin periodinane (DMP) (2.456 g, 5.790 mmol) at 0° C. The mixture was slowly warmed to RT and stirred for 4 h. The mixture was diluted with hexane, filtered through celite pad, followed by washing the celite pad with diethyl ether. The combined organic layer was washed with aqueous sodium thiosulfate solution and aqueous sodium bicarbonate solution, dried over anhydrous sodium sulphate, filtered, and concentrated to afford the title compound (0.600 g) as crude product which was taken in next step as such. LCMS: 201.9 [M+H]⁺.

### c) (S)-4-Bromo-2-methyl-6-((3-methylpiperidin-1-yl)methyl)pyridine (Intermediate-15)

The compound was prepared according to the procedure described for Intermediate-4 with appropriate variations in reactants, quantities of reagents, and solvents. LCMS: 285.1 [M+2+H]⁺.

### Intermediate 16. (S)-4-Bromo-2-((3-methylpiperidin-1-yl)methyl)-6-(trifluoro-methyl)pyridine

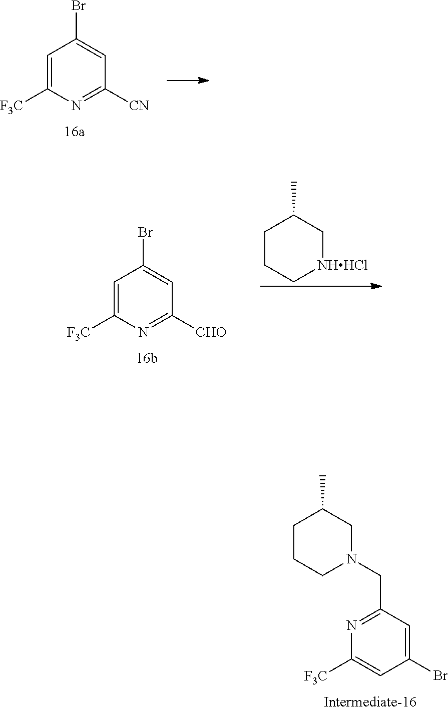

### a) 4-Bromo-6-(trifluoromethyl)picolinaldehyde (16b)

The compound was prepared from 4-bromo-6-(trifluoromethyl) picolinonitrile (prepared following WO 2010/100050) according to the procedure described Intermediate-6c with appropriate variations in reactants, quantities of reagents, and solvents. LCMS: 255.0 [M+H]⁺.

### b) (S)-4-Bromo-2-((3-methylpiperidin-1-yl)methyl)-6-(trifluoromethyl)pyridine (Intermediate 16)

The compound was prepared according to the procedure described for Intermediate-4 with appropriate variations in reactants, quantities of reagents, and solvents. LCMS: 339.0 [M+2+H]⁺.

### Intermediate-17. (S)-2-Chloro-6-methyl-4-((3-methylpiperidin-1-yl)methyl)-pyridine

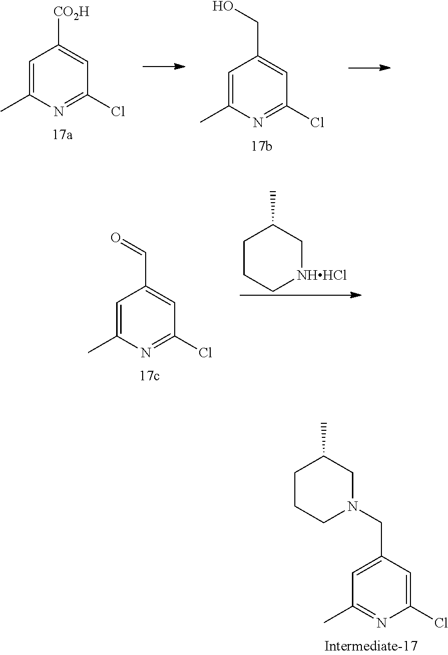

### a) (2-Chloro-6-methylpyridin-4-yl) methanol (17b)

To a solution of 2-chloro-6-methylisonicotinic acid (5.0 g, 29.14 mmol) in THF (50 ml) was added BH₃*THF (50 mL, 1 M in THF) dropwise at 0° C. Then the mixture was slowly warmed to RT and stirred for 16 h. The mixture was cooled to 0° C., quenched with water, and extracted with ethyl acetate. Then combined organic layer was washed with brine, dried over sodium sulphate, and concentrated. The crude product was purified by silica-gel flash column chromatography using 10-20% ethyl acetate in hexane as eluent to obtain the titled product (4.0 g, 87.10%). LCMS: 158.1 [M+H]⁺.

### b) 2-Chloro-6-methylisonicotinaldehyde (17c)

The compound was prepared according to the procedure described for preparing Intermediate-15c with appropriate variations in reactants, quantities of reagents, and solvents. LCMS: 156.0 [M+H]⁺.

### c) (S)-2-Chloro-6-methyl-4-((3-methylpiperidin-1-yl)methyl)pyridine (Intermediate-17)

The compound was prepared according to the procedure described for Intermediate-4 with appropriate variations in reactants, quantities of reagents, and solvents. LCMS: 239.1 [M+H]⁺.

### Intermediate-18 and Intermediate-19. (S)-2,6-Dichloro-4-((3-methylpiperidin-1-yl) methyl)pyridine (Intermediate 18) and (S)-2-Chloro-6-cyclopropyl-4-((3-methyl-piperidin-1-yl) methyl)pyridine (Intermediate 19)

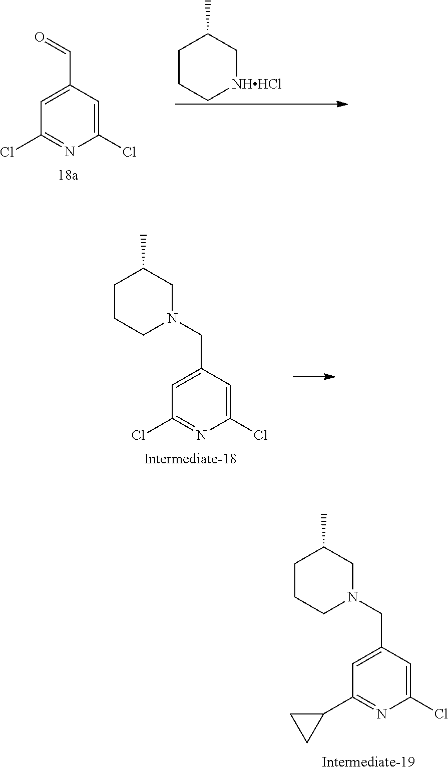

### a) (S)-2,6-Dichloro-4-((3-methylpiperidin-1-yl) methyl)pyridine (Intermediate-18)

The compound was prepared according to the procedure described for Intermediate-4 with appropriate variations in reactants, quantities of reagents, and solvents. LCMS: 261.1 [M+H]⁺.

### b) (S)-2-Chloro-6-cyclopropyl-4-((3-methylpiperidin-1-yl) methyl) pyridine (Intermediate-19)

The compound was prepared according to the procedure described for preparing Intermediate-11 with appropriate variations in reactants, quantities of reagents, and solvents. LCMS: 265.10 [M+H]⁺.

### Intermediate-20. 2-(6-Bromo-4-methyl-1H-indazol-1-yl)-N, N-dimethylethan-1-amine

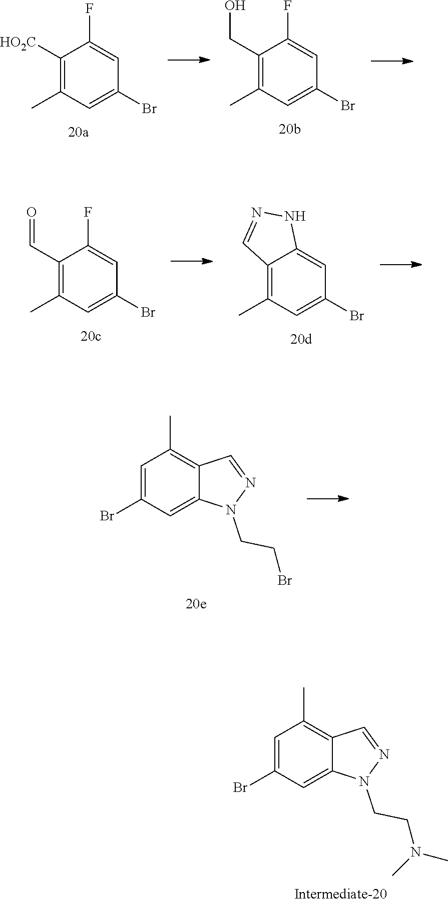 [SMILES: Cc1cc(Br)cc(F)c1C(=O)O ; Cc1cc(Br)cc(F)c1CO ; Cc1cc(Br)cc(F)c1C=O ; Cc1cc(Br)cc2[nH]ncc12 ; Cc1cc(Br)cc2c1cnn2CCBr ; Cc1cc(Br)cc2c1cnn2CCN(C)C]

### a) (4-Bromo-2-fluoro-6-methylphenyl)methanol (20b)

The compound was prepared according to the procedure described for Intermediate-17b with appropriate variations in reactants, quantities of reagents, and solvents. ¹H-NMR (400 MHz, CDCl₃) δ: 7.18 (brs, 1H) 7.12 (dd, 1H), 4.72 (s, 2H), 2.45 (s, 3H).

### b) 4-Bromo-2-fluoro-6-methylbenzaldehyde (20c)

The compound was prepared according to the procedure described for Intermediate-15c with appropriate variations in reactants, quantities of reagents, and solvents. ¹H-NMR (400 MHz, CDCl₃) δ 10.48 (s, 1H) 7.31-7.21 (m, 2H), 2.62 (s, 3H).

### c) 6-Bromo-4-methyl-1H-indazole (20d)

A mixture of 4-bromo-2-fluoro-6-methylbenzaldehyde (2.85 g, 13.13 mmol) and NH₂NH₂*H₂O (6.57 g, 131.31 mmol) in DMSO (20.0 mL) was stirred at 110° C. for 16 h. The mixture was cooled to RT, diluted with water, and extracted with ethyl acetate. The combined organic layer was dried over sodium sulphate, filtered, and concentrated to afford the title compound. (2.83 g, 97.88%). LCMS: 211.0 [M+H]⁺.

### d) 6-Bromo-1-(2-bromoethyl)-4-methyl-1H-indazole (20e)

To a solution of 6-bromo-4-methyl-1H-indazole (0.50 g, 2.37 mmol) and 1,2-dibromoethane (0.89 g, 4.74 mmol) in DMF (5.0 mL) was added CS₂CO₃ (1.16 g, 3.55 mmol) followed by stirring at RT for 12 h. The reaction mixture was quenched with water and extracted with ethyl acetate. The combined organic layer was dried over anhydrous sodium sulphate, filtered, and concentrated to afford the title compound (0.450 g, 59.73%). LCMS: 320.9 [M+H]⁺.

### e) 2-(6-Bromo-4-methyl-1H-indazol-1-yl)-N, N-dimethylethan-1-amine (Intermediate-20)

To a solution of 6-bromo-1-(2-bromoethyl)-4-methyl-1H-indazole (0.45 g, 1.42 mmol) and dimethylamine (0.64 g, 14.15 mmol) in DMF (5.0 mL) was added NaI (0.12 g, 1.41 mmol) followed by stirring at 70° C. for 5 h. The mixture was cooled to RT, diluted with water, and extracted with ethyl acetate. The combined organic layer was dried over sodium sulphate, filtered, and concentrated. The crude product was purified by silica-gel flash column chromatography using 10% MeOH in DCM as eluent to afford the titled compound. LCMS: 284.0 [M+H]⁺.

### Intermediate-21 and Intermediate-22. 1-(7-Bromo-5-(trifluoromethyl)-3,4-dihydroisoquinolin-2(1H)-yl) ethan-1-one (Intermediate-21) and 7-Bromo-2-ethyl-5-(trifluoromethyl)-1,2,3,4-tetrahydroisoquinoline (Intermediate-22)

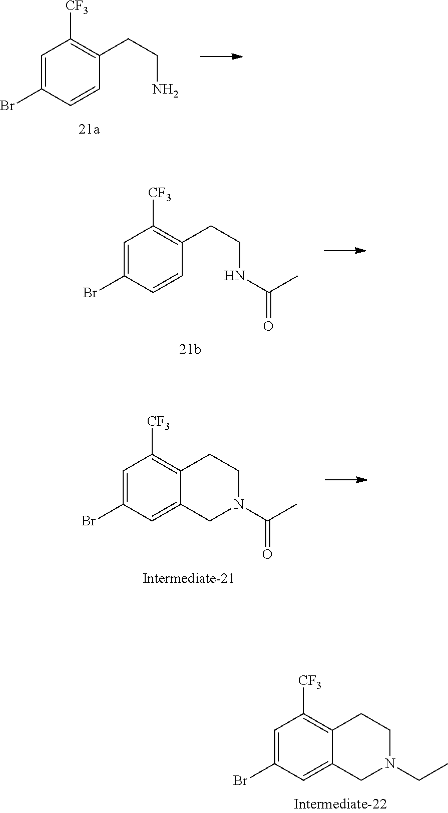 [SMILES: NCCc1ccc(Br)cc1C(F)(F)F ; CC(=O)NCCc1ccc(Br)cc1C(F)(F)F ; CC(=O)N1CCc2c(cc(Br)cc2C(F)(F)F)C1 ; CCN1CCc2c(cc(Br)cc2C(F)(F)F)C1]

### a) N-(4-Bromo-2-(trifluoromethyl)phenethyl) acetamide (21b)

To a solution 2-(4-bromo-2-(trifluoromethyl) phenyl) ethan-1-amine (1.8 g, 6.71 mmol (prepared according to WO 2021/032148) and triethylamine (1.36 g, 13.42 mmol) in dichloromethane (20.0 mL) was added acetyl chloride (0.53 g, 6.71 mmol) at 0° C. followed by stirring for 1 h. The reaction mixture was quenched with water and extracted with ethyl acetate. The combined organic layer was dried over anhydrous sodium sulphate, filtered, and concentrated. The crude product was purified by silica-gel flash column chromatography using 40% ethyl acetate in hexane as eluent to afford the title compound (1.5 g, 72%). LCMS: 311.9 [M+2+H]⁺.

### b) 1-(7-Bromo-5-(trifluoromethyl)-3,4-dihydroisoquinolin-2(1H)-yl) ethan-1-one (Intermediate-21)

To a solution N-(4-bromo-2-(trifluoromethyl) phenethyl) acetamide (1.45 g, 4.68 mmol) and formaldehyde (1.4 g, 46.7 mmol) in acetic acid (16.0 mL) was added sulfuric acid (0.45 mL) followed by stirring at 80° C. for 16 h. The reaction mixture was quenched with water and extracted with ethyl acetate. The combined organic layer was dried over anhydrous sodium sulphate, filtered, and concentrated. The crude product was purified by silica-gel flash column chromatography using 30% ethyl acetate in hexane as eluent to afford the title compound (0.650 g, 43%). LCMS: 323.9 [M+2+H]⁺.

### c) 7-Bromo-2-ethyl-5-(trifluoromethyl)-1,2,3,4-tetrahydroisoquinoline (Intermediate-22)

To a solution 1-(7-bromo-5-(trifluoromethyl)-3,4-dihydroisoquinolin-2(1H)-yl) ethan-1-one (0.05 g, 0.16 mmol) in dry THF (0.5 mL) was added BH₃*SMe₂ (0.18 mL, 0.38 mmol, 2.0 M in THF) at 0° C. Then the mixture was slowly warmed to RT and stirred for 2 h. The reaction mixture was cooled 0° C., quenched with saturated aqueous ammonium chloride and extracted with ethyl acetate. The combined organic layer was dried over anhydrous sodium sulphate, filtered, and concentrated to afford the title compound (0.04 g). LCMS: 310.9 [M+2+H]⁺.

### Intermediate 23. 3-((4-Methyl-4H-1,2,4-triazol-3-yl)methyl)-3-(3-oxoisoindo-lin-5-yl)cyclobutane-1-carbonitrile

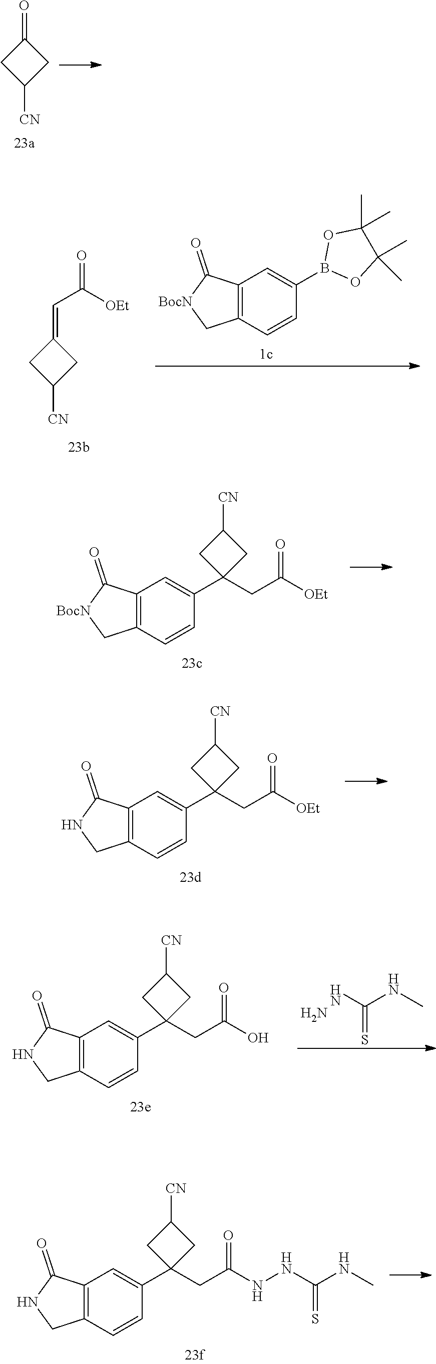

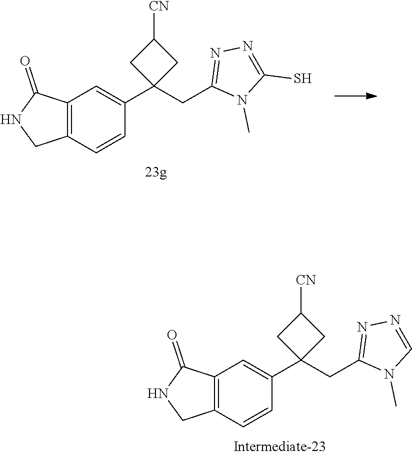 [SMILES: Cn1c(S)nnc1CC1(c2ccc3c(c2)C(=O)NC3)CC(C#N)C1 ; Cn1cnnc1CC1(c2ccc3c(c2)C(=O)NC3)CC(C#N)C1]

### a) Ethyl 2-(3-cyanocyclobutylidene)acetate (23b)

To a solution of 3-oxocyclobutane-1-carbonitrile (3.00 g, 31.545 mmol) in DCM (30.0 mL) was added ethyl 2-(triphenyl-15-phosphaneylidene) acetate (10.990 g, 31.545 mmol) and the mixture was stirred at RT for 15 h. The reaction was quenched with hexane and stirred for 10 min to obtain white precipitate. The precipitate was filtered and the filtrate was dried under vacuum to obtain the title product (2.72 g, 52.20%). ¹H-NMR (400 MHz, CDCl₃) δ: 5.73-5.72 (brm, 1H), 4.18 (q, 2H), 3.68-3.55 (m, 1H), 3.48-3.54 (m, 1H), 3.34-3.22 (m, 3H), 1.29 (t, 3H).

### b) tert-Butyl 6-(3-cyano-1-(2-ethoxy-2-oxoethyl)cyclobutyl)-1-oxoisoindoline-2-carboxylate (23c)

The compound was prepared according to the procedure described for Intermediate-1d with appropriate variations in reactants, quantities of reagents, and solvents. LCMS: 343.0 [M−56]⁺.

### c) Ethyl 2-(3-cyano-1-(3-oxoisoindolin-5-yl)cyclobutyl)acetate (23d)

The compound was prepared according to the procedure described for Intermediate-1e with appropriate variations in reactants, quantities of reagents, and solvents. LCMS: 297.1 [M−H]⁻

### d) 2-(3-Cyano-1-(3-oxoisoindolin-5-yl) cyclobutyl)acetic acid (23e)

To a solution of ethyl 2-(3-cyano-1-(3-oxoisoindolin-5-yl) cyclobutyl)acetate (2.64 g, 8.85 mmol) in THF(12.0 mL), MeOH (2.5 mL) and water (4.7 mL) was added LiOH (0.46 mg, 11.06 mmol) followed by stirring at RT for 4 h. The mixture was concentrated, residue was dissolved in water and pH was adjusted to acidic using 0.5 N of HCl. The precipitate was filtered, dried under vacuum to afford the title compound as white solid. LCMS: 271.1 [M+H]⁺.

### e) 2-(2-(3-Cyano-1-(3-oxoisoindolin-5-yl)cyclobutyl)acetyl)-N-methyl-hydrazine-1-carbothioamide (23f)

To a solution of 2-(3-cyano-1-(3-oxoisoindolin-5-yl)cyclobutyl)acetic acid (1.35 g, 4.99 mmol), N-methylhydrazinecarbothioamide (0.63 g, 5.99 mmol) and DIPEA (1.291 g, 9.990 mmol) in DMF (13.0 mL) was added HATU (1.41 g, 5.99 mmol) and the mixture was stirred at RT for 5 h. The reaction was quenched with ice cold water and stirred for 10 min to obtain brown precipitate. The precipitate was filtered and dried under vacuum to obtain the title product. LCMS: 358.1 [M+H]⁺.

### f) 3-((5-Mercapto-4-methyl-4H-1,2,4-triazol-3-yl)methyl)-3-(3-oxoisoindolin-5-yl)cyclobutane-1-carbonitrile (23g)

The compound was prepared according to the procedure described for Intermediate-1h with appropriate variations in reactants, quantities of reagents, and solvents. LCMS: 340.0 [M+H]⁺.

### g) 3-((4-Methyl-4H-1,2,4-triazol-3-yl)methyl)-3-(3-oxoisoindolin-5-yl)cyclobutane-1-carbonitrile (Intermediate-23)

The compound was prepared according to the procedure described for Intermediate-1 (last step) with appropriate variations in reactants, quantities of reagents, and solvents. LCMS: 308.1 [M+H]⁺.

### Intermediate-24. 6-(3-Methyl-1-(4-methyl-4H-1,2,4-triazol-3-yl)cyclobutyl)-isoindolin-1-one

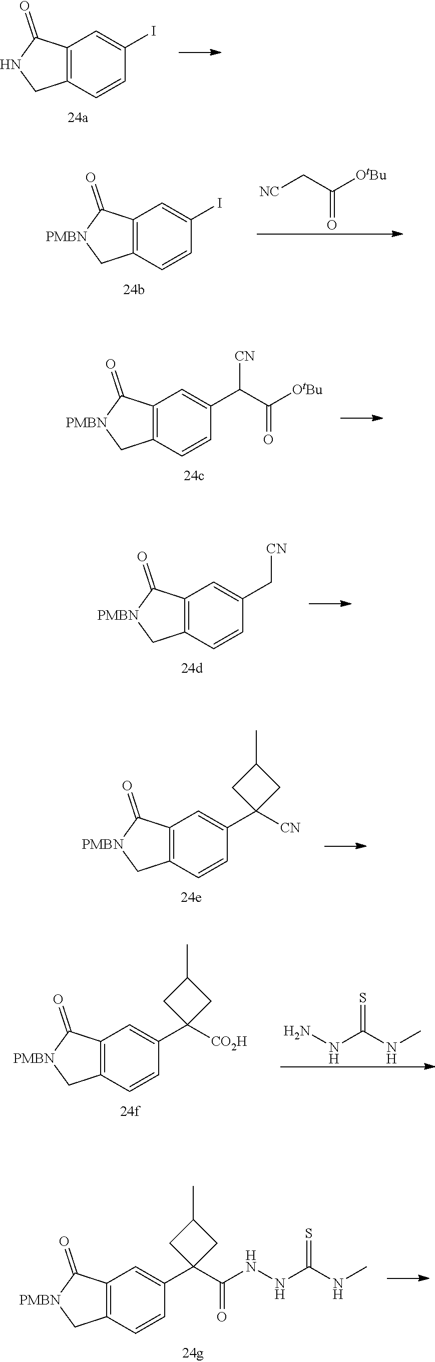

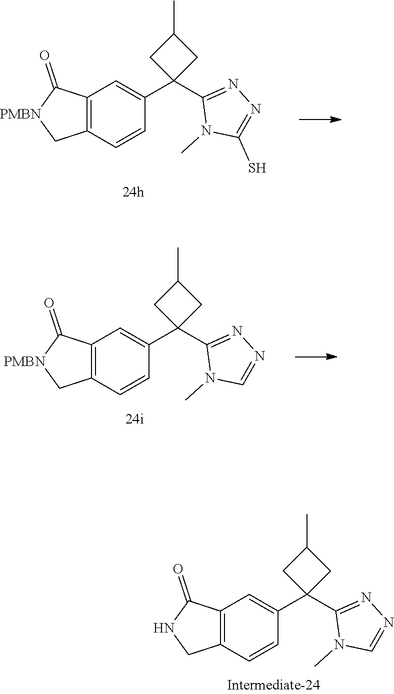

### a) 6-Iodo-2-(4-methoxybenzyl)isoindolin-1-one (24b)

To a solution of 6-iodoisoindolin-1-one (27 g, 104.23 mmol) and CS₂CO₃ (67.92 g, 208.45 mmol) in acetonitrile (270 mL) was added p-methoxybenzylchoride (PMBCl) (27 g, 104.23 mmol) dropwise over 30 min followed by stirring at 60° C. for 12 h. The mixture was cooled to RT and filtered through celite pad. The filtrate was concentrated and purified by silica-gel flash column chromatography using 20% ethyl acetate in hexane as eluent to afford the title compound (21 g, 53.13%). LCMS: 379.9 [M+H]⁺.

### b) tert-Butyl 2-cyano-2-(2-(4-methoxybenzyl)-3-oxoisoindolin-5-yl)acetate (24c)

To a degassed solution of 6-iodo-2-(4-methoxybenzyl) isoindolin-1-one (19.6 g, 51.69 mmol), tert-butyl 2-cyanoacetate (14.59 g, 103.38 mmol), picolinic acid (0.636 g, 5.160 mmol), and CS₂CO₃ (33.68 g, 103.37 mmol) in dioxane (200 mL) was added CuI (0.492 g, 2.580 mmol) followed by stirring at 80° C. for 12 h. The mixture was cooled to RT, filtered through celite pad, and concentrated. The product was diluted with water and extracted with ethyl acetate. The combined organic layer was dried over anhydrous sodium sulphate, filtered, and concentrated. The crude product was purified by silica-gel flash column chromatography using 30% ethyl acetate in hexane as eluent to afford the title compound (17 g, 83.81%). LCMS: 393.2 [M−H]⁺.

### c) 2-(2-(4-Methoxybenzyl)-3-oxoisoindolin-5-yl)acetonitrile (24d)

To a solution of tert-butyl 2-cyano-2-(2-(4-methoxybenzyl)-3-oxoisoindolin-5-yl)acetate (17.00 g, 43.317 mmol) in hexafluoroisopropanol (HFIP) (170.0 mL) was added 4 M HCl in dioxane (69 ml, 281.55 mmol) at 0° C. The mixture was slowly heated to 50° C. and stirred for 12 h. The mixture was cooled to RT, quenched with water, and extracted with ethyl acetate. The combined organic layer was dried over anhydrous sodium sulphate, filtered, and concentrated. The crude product was purified by silica-gel flash column chromatography using 50% ethyl acetate in hexane as eluent to afford the title compound (8.4 g, 66.34%). LCMS: 293.1 [M+H]⁺.

### d) 1-(2-(4-Methoxybenzyl)-3-oxoisoindolin-5-yl)-3-methylcyclobutane-1-carbonitrile (24e)

To a solution of 2-(2-(4-methoxybenzyl)-3-oxoisoindolin-5-yl) acetonitrile (4.00 g, 13.68 mmol) and KOH (1.919 g, 34.20 mmol) in DMSO (40.0 mL) was added 1,3-dibromo-2-methylpropane (2.95 g, 13.68 mmol) at 0° C. The mixture was slowly warmed to RT and stirred for 8 h. The reaction was quenched with water and extracted with ethyl acetate. The combined organic layer was dried over anhydrous sodium sulphate, filtered, and concentrated. The crude product was purified by silica-gel flash column chromatography using 40% ethyl acetate in hexane as eluent to afford the title compound (2.7 g, 56.97%). LCMS: 347.1 [M+H]⁺.

### e) 1-(2-(4-Methoxybenzyl)-3-oxoisoindolin-5-yl)-3-methylcyclobutane-1-carboxylic acid (24f)

To a solution of 1-(2-(4-methoxybenzyl)-3-oxoisoindolin-5-yl)-3-methylcyclo-butane-1-carbonitrile (1.7 g, 4.91 mmol) and KOH (2.49 g, 44.16 mmol) in EtOH (10.0 mL), water (2.5 mL) was added followed by stirring at 110° C. for 12 h. The mixture was cooled to RT, quenched with ice cold water and extracted with 20% MeOH in DCM. The combined organic layer was dried over anhydrous sodium sulphate, filtered, and concentrated to afford the title compound (1.5 g, 83.65%). LCMS: 366.1 [M+H]⁺.

### f) 2-(1-(2-(4-Methoxybenzyl)-3-oxoisoindolin-5-yl)-3-methylcyclobutane-1-carbonyl)-N-methylhydrazine-1-carbothioamide (24g)

The compound was prepared according to the procedure described for Intermediate-23f with appropriate variations in reactants, quantities of reagents, and solvents. LCMS: 453.1 [M+H]⁺.

### g) 6-(1-(5-Mercapto-4-methyl-4H-1,2,4-triazol-3-yl)-3-methylcyclobutyl)-2-(4-methoxybenzyl)isoindolin-1-one (24h)

The compound was prepared according to the procedure described for Inter-mediate-1h with appropriate variations in reactants, quantities of reagents, and solvents. LCMS: 435.2 [M+H]⁺.

### h) 2-(4-Methoxybenzyl)-6-(3-methyl-1-(4-methyl-4H-1,2,4-triazol-3-yl)cyclo-butyl)isoindolin-1-one (24i)

The compound was prepared according to the procedure described for Intermediate-1 (last step) with appropriate variations in reactants, quantities of reagents, and solvents. LCMS: 403.2 [M+H]⁺.

### i) 6-(3-Methyl-1-(4-methyl-4H-1,2,4-triazol-3-yl)cyclobutyl)isoindolin-1-one (Intermediate 24)

A solution of 2-(4-methoxybenzyl)-6-(3-methyl-1-(4-methyl-4H-1,2,4-triazol-3-yl)cyclobutyl)isoindolin-1-one (0.5 g, 1.24 mmol) in TFA (10.0 mL) was irradiated in microwave at 100° C. for 4 h. The mixture was concentrated, diluted with DCM, added solid NaHCO₃ and stirred for 2 h. The organic layer was filtered through sintered funnel, dried over anhydrous sodium sulphate, filtered, and concentrated to afford the title compound. LCMS: 283.1 [M+H]⁺.

### Intermediate-25. 2-Chloro-4-((4,4-difluoro-3-methylpiperidin-1-yl)methyl)-6-(trifluoromethyl)pyridine

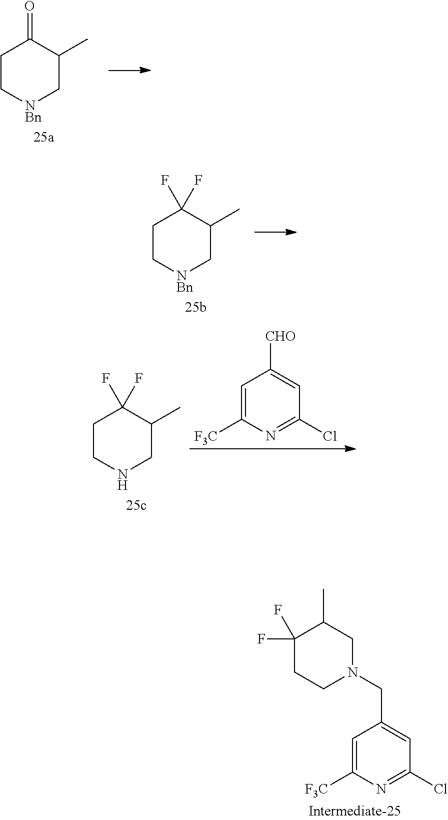

### a) 1-Benzyl-4,4-difluoro-3-methylpiperidine (25b)

To a solution of 1-benzyl-3-methylpiperidin-4-one (1.0 g, 4.918 mmol) in dichloromethane (10 mL) was added (diethylamino) sulphur trifluoride (3.96 g, 24.59 mmol) followed by stirring at RT for 16 h. The reaction mixture was quenched by saturated sodium bicarbonate and extracted with ethyl acetate. The combined organic layer was dried over anhydrous sodium sulphate, filtered, and concentrated. The crude product was purified by silica-gel flash column chromatography using 10-20% ethyl acetate in hexane as eluent to obtain the title product (0.9 g, 81.22%). LCMS: 226.1 [M+H]⁺.

### b) 4,4-Difluoro-3-methylpiperidine (25c)

To a solution of 1-benzyl-4,4-difluoro-3-methylpiperidine (0.9 g, 3.995 mmol) in ethanol (10 ml) was added Pd/C (0.43 g, 0.400 mmol) followed by stirring at RT under hydrogen atmosphere for 24 h. The mixture was filtered through celite pad followed by washing the celite pad with dichloromethane. The filtrate was concentrated to afford the title product (0.750 g, 75%). LCMS: 136.1 [M+H]⁺.

### c) 2-Chloro-4-((4,4-difluoro-3-methylpiperidin-1-yl)methyl)-6-(trifluoromethyl)-pyridine (Intermediate-25)

The compound was prepared according to the procedure described for Intermediate-5 with appropriate variations in reactants, quantities of reagents, and solvents. LC-MS: 331.0 [M+2+H]⁺.

### Intermediate-26. (S)-(2-Chloro-6-(trifluoromethyl)pyridin-4-yl)(3-hydroxy-pyrrolidin-1-yl)methanone

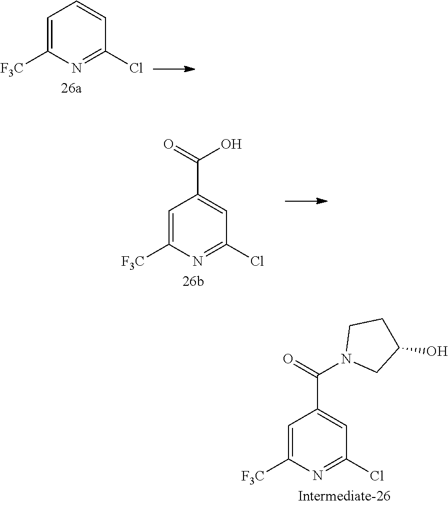 [SMILES: FC(F)(F)c1cccc(Cl)n1 ; O=C(O)c1cc(Cl)nc(C(F)(F)F)c1 ; O=C(c1cc(Cl)nc(C(F)(F)F)c1)N1CC[C@H](O)C1]

### a) 2-Chloro-6-(trifluoromethyl)isonicotinic acid (26b)

To a solution of 2-chloro-6-(trifluoromethyl) pyridine (5.0 g, 27.542 mmol) in dry THF (100.0 ml) was added 2,2,6,6-tetramethylpiperidinylmagnesium chloride lithium chloride complex (1.0 M solution in THF, 33.0 ml, 33.05 mmol) at RT followed by stirring for 1 h. The mixture was cooled to −78° C. and dry ice (16.0 g) was added portionwise. The mixture was slowly warmed to RT and stirred for 16 h. The reaction mixture was quenched with water at 0° C. and extracted with ethyl acetate. The combined organic layer was dried over anhydrous sodium sulphate, filtered, concentrated, and purified by silica-gel flash column chromatography using 5-10% methanol in dichloromethane as eluent to afford the title compound (0.700 g, 11.27%); LCMS: 225.9 [M+H]⁺.

### b) (S)-(2-Chloro-6-(trifluoromethyl)pyridin-4-yl)(3-hydroxypyrrolidin-1-yl)methanone (Intermediate-26)

To a solution of 2-chloro-6-(trifluoromethyl)isnicotinic acid (0.60 g, 2.66 mmol) in DCM (1.0 ml) was added thionyl chloride(0.949 g, 7.98 mmol). The mixture was refluxed at 55° C. for 2 h. The mixture was then cooled to RT and evaporated under argon. The obtained crude acid chloride was dissolved in dichloromethane (6.0 ml), and triethyl amine (0.251 g, 2.47 mmol) was added followed by (S)-pyrrolidin-3-ol (0.157 g, 1.80 mmol). The mixture was stirred for 12 h at RT. The reaction mixture was quenched with water and extracted with dichloromethane. The combined organic layer was dried over anhydrous sodium sulphate, filtered, concentrated, and purified by silica-gel flash column chromatography using 0-5% methanol in dichloromethane as eluent to afford the title compound (0.230 g, 34.63%); LCMS: 295.0 [M+H]⁺.

### Intermediate-27. (3S)-1-(1-(3-bromo-5-methylphenyl) ethyl)-3-methyl-piperidine

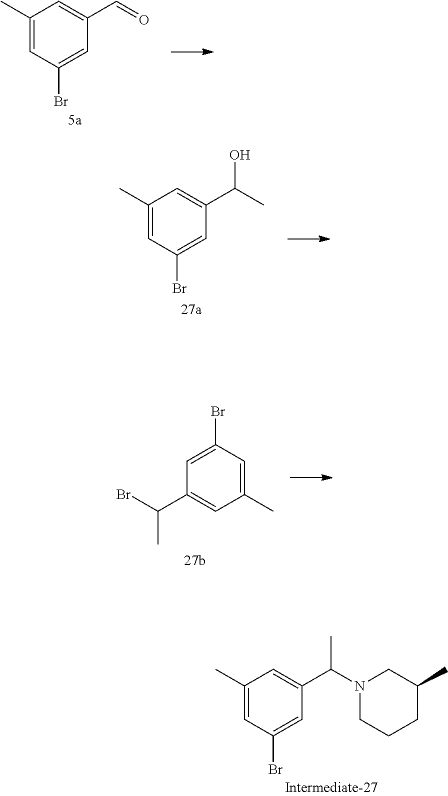

### a) 1-(3-Bromo-5-methylphenyl) ethan-1-ol (27a)

To a solution of 3-bromo-5-methylbenzaldehyde (3.0 g, 15.07 mmol) in THF (30 mL) was added methyl magnesium bromide (60.28 ml, 60.28 mmol, 1 M in THF) dropwise at −78° C. followed by stirring for 1 h. The reaction mixture was quenched with saturated aqueous ammonium chloride solution and extracted with ethyl acetate. The combined organic layer was dried over anhydrous sodium sulphate, filtered, and concentrated. The crude product was purified by silica-gel flash column chromatography using 20-30% ethyl acetate in hexane as eluent to afford the title compound (1.3 g, 40%). ¹H NMR (DMSO, 400 MHz): 7.31 (s, 1H), 7.25-7.21 (m, 1H), 7.14 (d, 1H), 5.24 (d, 1H), 4.69-4.64 (m, 1H), 2.29 (s, 3H), 1.99 (d, 3H).

### b) 1-Bromo-3-(1-bromoethyl)-5-methylbenzene (27b)

To a solution of 1-(3-bromo-5-methylphenyl) ethan-1-ol (0.65 g, 3.02 mmol) and triphenyl phosphine (0.95 g, 3.62 mmol) in DCM (10 mL) was added CBr₄ (1.0 g, 3.62 mmol) followed by stirring at RT for 3 h. The reaction was quenched with water and extracted with DCM. The combined organic layer was dried over anhydrous sodium sulphate, filtered, and concentrated. The crude product was purified by silica-gel flash column chromatography using 5% ethyl acetate in hexane as eluent to afford the title compound (0.5 g, 59%). ¹H NMR (CDCl₃, 400 MHz): 7.38 (s, 1H), 7.26 (d, 1H), 7.16 (s, 1H), 5.01 (q, 1H), 2.33 (s, 3H), 2.01 (d, 3H).

### c) (3S)-1-(1-(3-Bromo-5-methylphenyl) ethyl)-3-methylpiperidine (Intermediate-27)

To a solution of (S)-3-methylpiperidine hydrochloride (0.220 g, 1.61 mmol), tetrabutylammonium iodide (0.399 g, 1.07 mmol), and DIPEA (0.558 g, 4.31 mmol) in DCM (6 mL) was added 1-bromo-3-(1-bromoethyl)-5-methyl benzene (0.3 g, 1.07 mmol) followed by stirring at RT for 12 h. The reaction was quenched with water and extracted with DCM. The combined organic layer was dried over anhydrous sodium sulphate, filtered, and concentrated. The crude product was purified by silica-gel flash column chromatography using 10% ethyl acetate in hexane as eluent to afford the title compound (0.16 g, 50%). LCMS: 298.1 [M+H]⁺.

### Intermediate-28. 2-Chloro-4-((2-methyl-1H-imidazol-1-yl) methyl)-6-(tri-fluoromethyl)pyridine

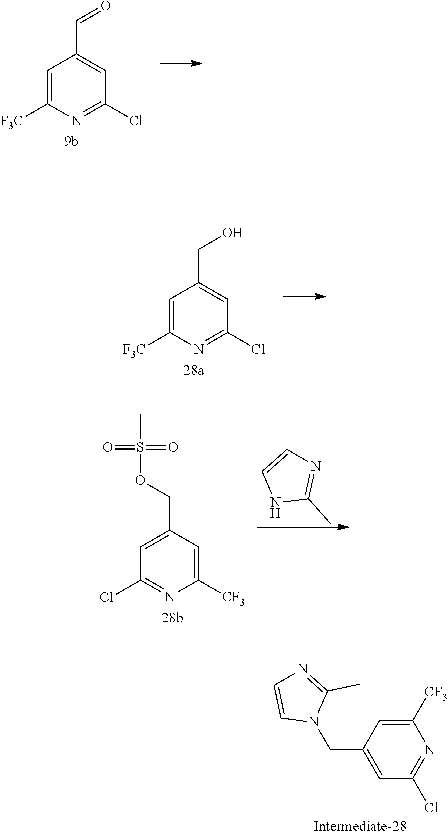

### a) (2-Chloro-6-(trifluoromethyl) pyridin-4-yl) methanol (28a)

To a solution of 2-chloro-6-(trifluoromethyl)isonicotinaldehyde (1.0 g, 4.77 mmol) in THF (10 mL) was added sodium borohydride (0.36 g, 9.54 mmol) portion wise at 0° C. The mixture was slowly warmed to RT and stirred for 2 h. The reaction was quenched with aqueous ammonium chloride solution and extracted with ethyl acetate. The combined organic layer was dried over anhydrous sodium sulphate, filtered, concentrated, and purified by silica-gel flash column chromatography using 20-30% ethyl acetate in hexane as eluent to afford the title compound (0.97 g, 96.08%). LCMS: 212.0 [M+H]⁺.

### b) (2-Chloro-6-(trifluoromethyl) pyridin-4-yl)methyl methane sulfonate (28b)

To a solution of (2-chloro-6-(trifluoromethyl)pyridin-4-yl) methanol (0.35 g, 1.68 mmol) and triethylamine (0.51 g, 5.06 mmol) in DCM (6 mL) was added methanesulfonyl chloride (0.26 mL, 3.37 mmol) at 0° C. and followed by stirring the reaction mixture at RT for 1 h. The reaction mixture was quenched with water and extracted with DCM. The combined organic layer was dried over anhydrous sodium sulphate, filtered, and concentrated to afford the title compound (0.6 g) as crude product which was used in next step as such. LCMS: 289.9 [M+H]⁺.

### c) 2-Chloro-4-((2-methyl-1H-imidazol-1-yl) methyl)-6-(trifluoromethyl)pyridine (Intermediate 28)

To a solution of (2-chloro-6-(trifluoromethyl)pyridin-4-yl) methyl methane sulfonate (0.50 g, 1.72 mmol) in THF (7 mL) was added cesium carbonate (1.12 g, 3.45 mmol) and 2-methyl-1H-imidazole (0.42 g, 5.17 mmol) followed by stirring at RT for 12 h. The reaction mixture was quenched with water and extracted with ethyl acetate. The combined organic layer was dried over anhydrous sodium sulphate, filtered, and concentrated. The crude product was purified by silica-gel flash column chromatography using 5-10% methanol in dichloromethane as eluent to afford the title compound (0.21 g, 44%). LCMS: 276.0 [M+H]⁺.

The following intermediates were prepared according to the procedure described for Intermediate-28 from the starting materials indicated in the Table with appropriate variations in reactants, quantities of reagents, coupling methods and solvents. The characterization data of the compounds are also summarized in the Table.

| | | LCMS | |
|---|---|---|---|
| No. | Structure | [M + H]⁺ | Starting material |
| Int-29 | 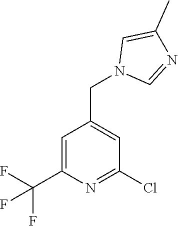 [SMILES: Cc1cn(Cc2cc(Cl)nc(C(F)(F)F)c2)cn1] | 276.0 | 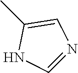 |
| Int-30 | 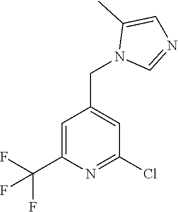 [SMILES: Cc1cncn1Cc1cc(Cl)nc(C(F)(F)F)c1] | 276.0 | 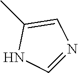 |
| Int-31 | 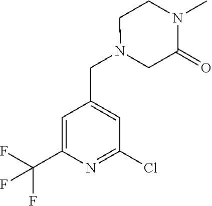 [SMILES: CN1CCN(Cc2cc(Cl)nc(C(F)(F)F)c2)CC1=O] | 308.0 | 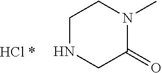 |
| Int-32 | 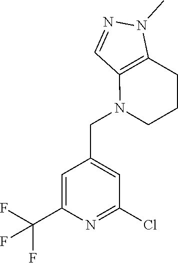 [SMILES: Cn1ncc2c1CCCN2Cc1cc(Cl)nc(C(F)(F)F)c1] | 332.1 | 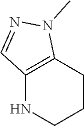 |
| Int-33 | 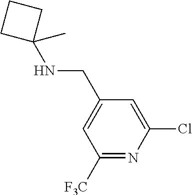 [SMILES: CC1(NCc2cc(Cl)nc(C(F)(F)F)c2)CCC1] | 279.0 | 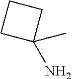 |
| Int-34 | 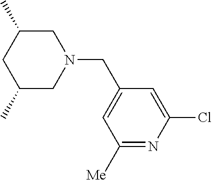 [SMILES: Cc1cc(CN2C[C@H](C)C[C@H](C)C2)cc(Cl)n1] | 255.1 | 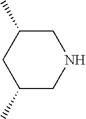 |
| Int-35 | 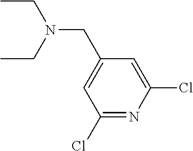 | 233.0 | 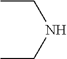 |

### Intermediate-36. 2-Chloro-4-((2,5-dimethylpiperidin-1-yl)methyl)-6-(trifluoro-methyl)pyridine

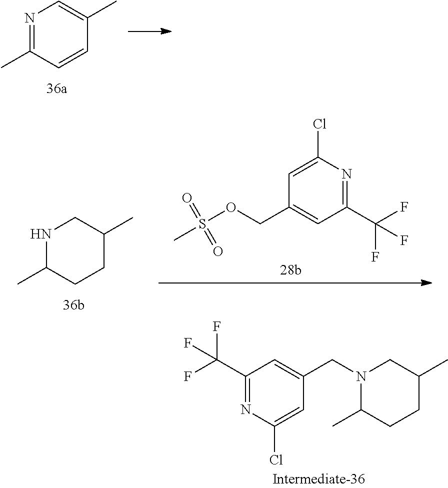

### a) 2,5-Dimethylpiperidine (36b)

To a solution of 2,5-dimethylpyridine (0.5 g, 4.66 mmol) in TFA (10 mL) was added platinum oxide (0.10 g, 0.46 mmol) in autoclave vessel followed by stirring at RT under hydrogen gas of 80 psi pressure for 12 h. The mixture was filtered through celite pad and concentrated. The pH of the crude product was adjusted to 7 with 7 M methanolic ammonia solution, then concentrated and purified by preparative HPLC to afford the title compound (0.4 g, 75.73%) LCMS: 114.1 [M+H]⁺.

### b) 2-Chloro-4-((2,5-dimethylpiperidin-1-yl)methyl)-6-(trifluoromethyl)pyridine (Intermediate-36)

The compound was prepared according to the procedure described for Intermediate-28 with appropriate variations in reactants, quantities of reagents, and solvents. LCMS: 307.2 [M+H]⁺.

### Intermediate-37. 2-(3-Bromo-5-(trifluoromethyl)phenyl)-N-ethylethan-1-amine

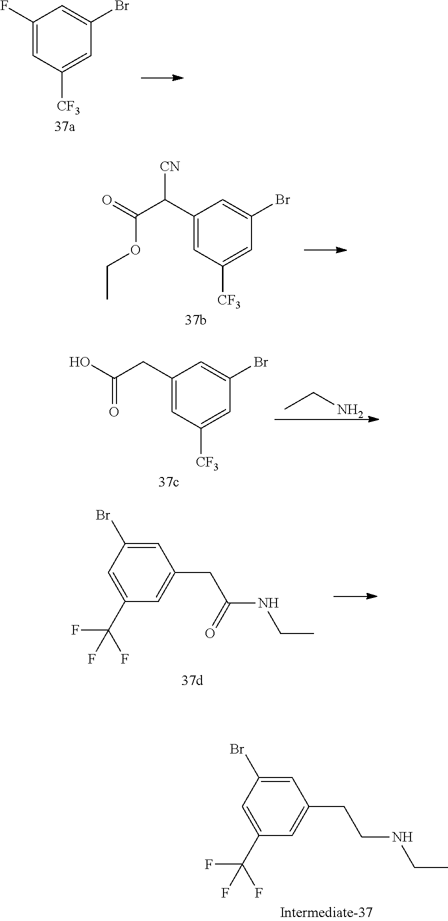

### a) Ethyl 2-(3-bromo-5-(trifluoromethyl)phenyl)2-cyanoacetate (37b)

To a solution of sodium hydride (1.2 g, 53.08 mmol) in N-methyl-2-pyrrolidone (NMP) (12 mL) was added ethyl 2-cyanoacetate (6.00 g, 53.08 mmol) followed by 1-bromo-3-fluoro-5-(trifluoromethyl)benzene (4.3 g, 17.69 mmol). The mixture was stirred at 110° C. for 12 h. The mixture was cooled to 0° C., quenched with 2 N HCl and extracted by ethyl acetate. The combined organic layer was dried over anhydrous sodium sulphate, filtered, concentrated and purified through by silica-gel flash column chromatography using 3-4% ethyl acetate in hexane as eluent to afford the title compound (3.5 g, 58.85%); LCMS: 335.9 [M+H]⁺.

### b) 2-(3-Bromo-5-(trifluoromethyl)phenyl)acetic acid (37c)

To a solution of ethyl 2-(3-bromo-5-(trifluoromethyl)phenyl)-2-cyanoacetate (3.4 g, 10.11 mmol) in water (35 mL) was added aqueous sodium hydroxide (2.0 g, 50.58 mmol) followed by stirring at 95° C. for 6 h. The mixture was concentrated, and pH was adjusted to 3-4 using 1 N HCl. The obtained precipitate was filtered through sintered funnel and dried under vacuum to afford the title compound (1.8 g, 62.8%). ¹H NMR (DMSO-d6, 400 MHz): 12.80-12.50 (bs, 1H), 7.85 (s, 1H), 7.84 (s, 1H), 7.69 (s, 1H), 3.75 (s, 2H).

### c) 2-(3-Bromo-5-(trifluoromethyl)phenyl)-N-ethylacetamide (37d)

To a solution of ethanamine (0.08 g, 1.766 mmol) and 2-(3-bromo-5-(trifluoro-methyl)phenyl)acetic acid (0.5 g, 1.76 mmol) in MeCN (6 mL) was added 1-hydroxy-benzotriazol (HOBT) (0.26 g, 1.76 mmol) and 1-ethyl-3-[3-dimethylaminopropyl]-carbodiimide (EDCl) (0.33 g, 1.76 mmol) followed by stirring at RT for 16 h. The mixture was diluted with water and extracted with DCM. The combined organic layer was washed with 1 N HCl and sodium bicarbonate solution. The organic layer was dried over anhydrous sodium sulphate, filtered, and concentrated to obtain the title compound (0.4 g). LCMS: 312.0 [M+H]⁺.

### d) 2-(3-Bromo-5-(trifluoromethyl)phenyl)-N-ethylethan-1-amine (Intermediate-37)

To a solution of 2-(3-bromo-5-(trifluoromethyl)phenyl)-N-ethylacetamide (0.25 g, 0.80 mmol) in dry THF (3 mL) was added borane dimethylsulfide complex (1.61 mL, 3.22 mmol, 2 M in THF) at 0° C. The mixture was slowly warned to RT and stirred for 1 h. The reaction mixture was quenched with ice-cold water and extracted with ethyl acetate. The combined organic layer was dried over anhydrous sodium sulphate, filtered, concentrated, and purified by silica-gel flash column chromatography using 5% MeOH in DCM as eluent to afford the title compound (0.18 g, 75%). LCMS: 298.0 [M+H]⁺.

### Intermediate-38. (S)-1-((6-Bromobenzofuran-4-yl)methyl)-3-methylpiperidine

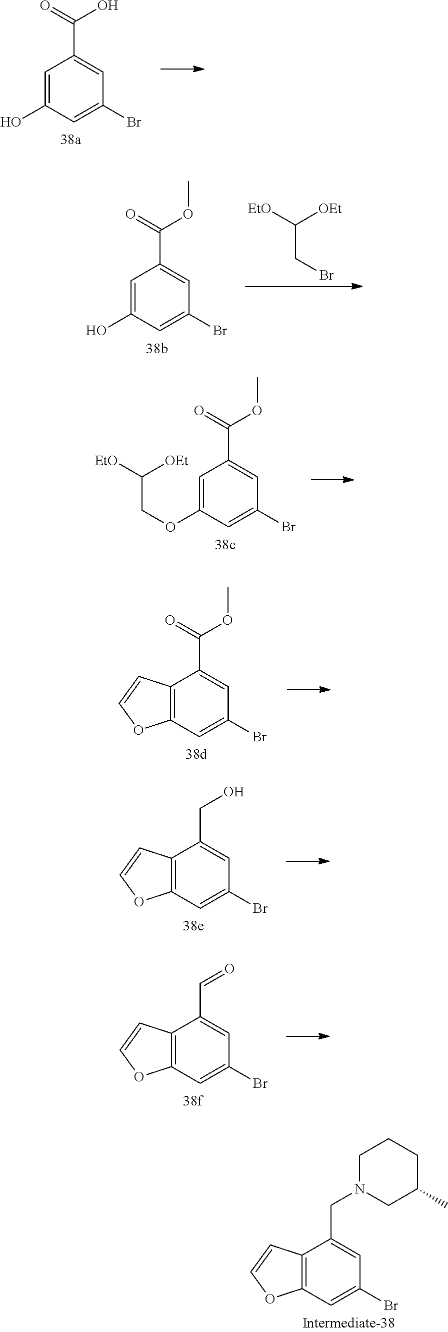

### a) Methyl 3-bromo-5-hydroxybenzoate (38b)

To a solution of 3-bromo-5-hydroxybenzoic acid (10 g, 46.08 mmol) in MeOH (50 mL) was added H₂SO₄ (18.06 g, 184.30 mmol) followed by stirring for 12 h at 70° C. The mixture was concentrated, diluted with water, and extracted with ethyl acetate. The combined organic layer was dried over anhydrous sodium sulphate, filtered, and concentrated. The crude product was purified by silica-gel flash column chromatography using 0-30% ethyl acetate in hexane as eluent to afford the title compound (10 g, 93.93%). LCMS: 233.1 [M+H]⁺.

### b) Methyl 3-bromo-5-(2,2-diethoxyethoxy)benzoate (38c)

To a solution of methyl 3-bromo-5-hydroxybenzoate (9.0 g, 38.95 mmol) and 2-bromo-1,1-diethoxyethane (15.3 g, 77.92 mmol) in DMF (60 mL) was added cesium carbonate (25.38 g, 77.90 mmol) followed by stirring for 12 h at 95° C. The reaction was quenched with water and extracted with ethyl acetate. The combined organic layer was dried over anhydrous sodium sulphate, filtered, and concentrated. The crude product was purified by silica-gel flash column chromatography using 0-30% ethyl acetate in hexane as eluent to afford the title compound (8.0 g, 59.15%). LCMS: 347.2 [M+H]⁺.

### c) Methyl 6-bromobenzofuran-4-carboxylate (38d)

To a solution of methyl 3-bromo-5-(2,2-diethoxyethoxy) benzoate (4.0 g, 11.51 mmol) in toluene (30 mL) was added polyphosphoric acid (PPA) (3.38 g, 34.56 mmol) followed by stirring for 6 h at 115° C. The mixture was cooled to RT, quenched with water, and extracted with ethyl acetate. The combined organic layer was dried over anhydrous sodium sulphate, filtered, and concentrated. The crude product was purified by silica-gel flash column chromatography using 10-30% ethyl acetate in hexane as eluent to afford the title compound (2.5 g, 85.08%). ¹H NMR (400 MHz, CDCl₃) δ: 8.13 (s, 1H), 7.88 (s, 1H), 7.73 (d, 1H), 7.37 (d, 1H), 4.07 (s, 3H).

### d) (6-Bromobenzofuran-4-yl)methanol (38e)

To a solution of methyl 6-bromobenzofuran-4-carboxylate (1.5 g, 5.88 mmol) in dry THF (10 mL) was added LiBH₄ (11.7 mL, 11.760 mmol, 1 M in THF) at −78° C. followed by stirring for 3 h. The reaction was quenched with saturated ammonium chloride solution and extracted with ethyl acetate. The combined organic layer was dried over anhydrous sodium sulphate, filtered, and concentrated. The crude product was purified by silica-gel flash column chromatography using 10-30% ethyl acetate in hexane as eluent to afford the title compound (1.2 g, 88.97%). ¹H NMR (400 MHz, DMSO-d6) δ: 8.03 (s, 1H), 7.84 (s, 1H), 7.55 (d, 1H), 7.01 (d, 1H), 5.47 (t, 1H), 4.88 (d, 2H).

### e) 6-Bromobenzofuran-4-carbaldehyde (38f)

To a solution (6-bromobenzofuran-4-yl)methanol (1.00 g, 4.40 mmol) in dichloromethane (20.0 mL) was added Dess-Martin periodinane (DMP) (3.76 g, 8.80 mmol) at 0° C. The mixture was slowly warmed to RT and stirred for 4 h. The mixture was diluted with hexane, filtered through celite pad, followed by washing the celite pad with diethyl ether. The combined organic layers were washed with aqueous sodium thiosulfate solution and aqueous sodium bicarbonate solution, dried over anhydrous sodium sulphate, filtered, and concentrated to afford the title compound (1.0 g) as crude product which was used in next step as such. ¹H NMR (400 MHz, DMSO-d6) δ: 10.17 (s, 1H), 7.99 (s, 1H), 7.89 (d, 1H), 7.71 (d, 1H), 5.51 (d, 1H).

### f) (S)-1-((6-Bromobenzofuran-4-yl)methyl)-3-methylpiperidine (Intermediate-38)

The compound was prepared according to the procedure described for Intermediate-2 with appropriate variations in reactants, quantities of reagents, and solvents. LCMS: 309.23 [M+H]⁺.

The following intermediates were prepared according to the procedure described for Intermediate 4 (procedure A) and 5 (procedure B) with appropriate starting materials, variations in reactants, quantities of reagents, coupling methods and solvents. The characterization data of the compounds are also summarized in the Table.

| | | | LCMS |
|---|---|---|---|
| No. | Structure | Procedure | [M + H]⁺ |
| Int-39 | 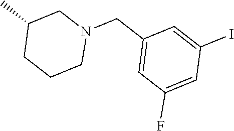 [SMILES: C[C@H]1CCCN(Cc2cc(F)cc(I)c2)C1] | A | 333.9 |
| Int-40 | 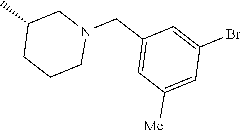 [SMILES: Cc1cc(Br)cc(CN2CCC[C@H](C)C2)c1] | A | 282.1 |
| Int-41 | 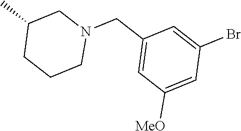 [SMILES: COc1cc(Br)cc(CN2CCC[C@H](C)C2)c1] | A | 298.0 |
| Int-42 | 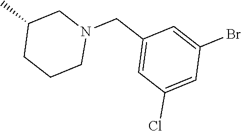 [SMILES: C[C@H]1CCCN(Cc2cc(Cl)cc(Br)c2)C1] | A | 303.85 |
| Int-43 | 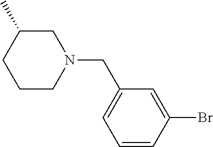 | A | 270.0 |
| Int-44 | 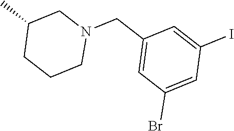 [SMILES: C[C@H]1CCCN(Cc2cc(Br)cc(I)c2)C1] | A | 393.9 |
| Int-45 | 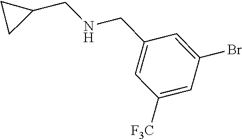 [SMILES: FC(F)(F)c1cc(Br)cc(CNCC2CC2)c1] | B | 309.9 |
| Int-46 | 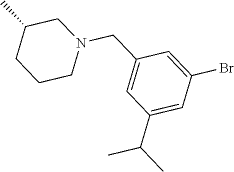 [SMILES: CC(C)c1cc(Br)cc(CN2CCC[C@H](C)C2)c1] | A | 312.0 |
| Int-47 | 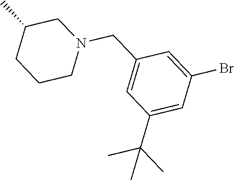 [SMILES: C[C@H]1CCCN(Cc2cc(Br)cc(C(C)(C)C)c2)C1] | A | 326.1 |
| Int-48 | 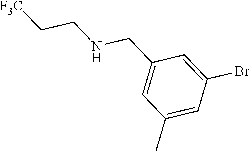 [SMILES: Cc1cc(Br)cc(CNCCC(F)(F)F)c1] | B | 298.0 |
| Int-49 | 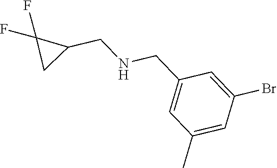 [SMILES: Cc1cc(Br)cc(CNCC2CC2(F)F)c1] | A | 290.0 |
| Int-50 | 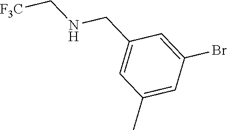 | B | 284.0 |
| Int-51 | 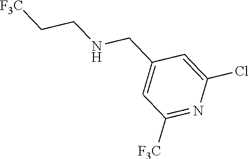 [SMILES: FC(F)(F)CCNCc1cc(Cl)nc(C(F)(F)F)c1] | A | 307.0 |
| Int-52 | 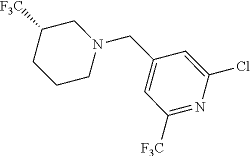 [SMILES: FC(F)(F)c1cc(CN2CCC[C@H](C(F)(F)F)C2)cc(Cl)n1] | A | 347.0 |
| Int-53 | 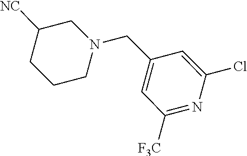 [SMILES: N#CC1CCCN(Cc2cc(Cl)nc(C(F)(F)F)c2)C1] | B | 303.95 |
| Int-54 | 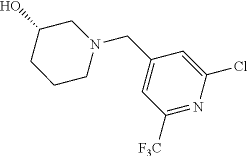 [SMILES: O[C@H]1CCCN(Cc2cc(Cl)nc(C(F)(F)F)c2)C1] | A | 295.0 |
| Int-55 | 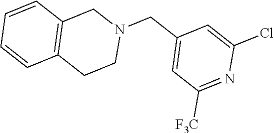 [SMILES: FC(F)(F)c1cc(CN2CCc3ccccc3C2)cc(Cl)n1] | B | 327.1 |
| Int-56 | 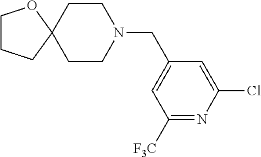 [SMILES: FC(F)(F)c1cc(CN2CCC3(CCCO3)CC2)cc(Cl)n1] | A | 335.0 |
| Int-57 | 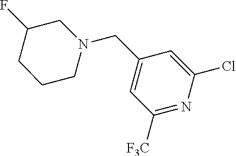 [SMILES: FC1CCCN(Cc2cc(Cl)nc(C(F)(F)F)c2)C1] | A | 297.0 |
| Int-58 | 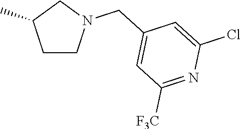 [SMILES: C[C@H]1CCN(Cc2cc(Cl)nc(C(F)(F)F)c2)C1] | A | 279.0 |
| Int-59 | 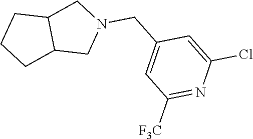 [SMILES: FC(F)(F)c1cc(CN2CC3CCCC3C2)cc(Cl)n1] | B | 305.1 |
| Int-60 | 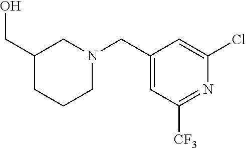 [SMILES: OCC1CCCN(Cc2cc(Cl)nc(C(F)(F)F)c2)C1] | B | 309.2 |
| Int-61 | 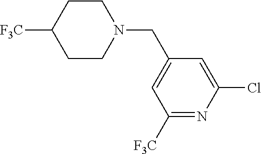 [SMILES: FC(F)(F)c1cc(CN2CCC(C(F)(F)F)CC2)cc(Cl)n1] | B | 347.0 |
| Int-62 | 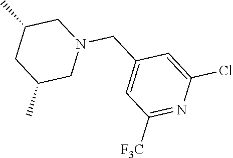 [SMILES: C[C@@H]1C[C@H](C)CN(Cc2cc(Cl)nc(C(F)(F)F)c2)C1] | B | 307.1 |
| Int-63 | 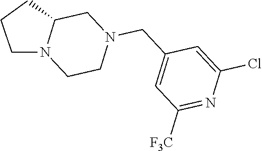 [SMILES: FC(F)(F)c1cc(CN2CCN3CCC[C@@H]3C2)cc(Cl)n1] | B | 320.1 |
| Int-64 | 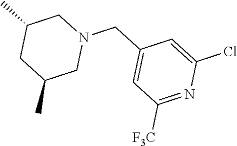 [SMILES: C[C@H]1C[C@H](C)CN(Cc2cc(Cl)nc(C(F)(F)F)c2)C1] | B | 307.0 |
| Int-65 | 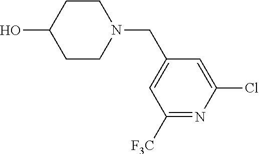 [SMILES: OC1CCN(Cc2cc(Cl)nc(C(F)(F)F)c2)CC1] | B | 295.0 |
| Int-66 | 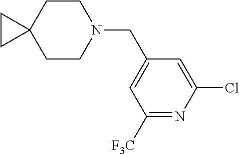 [SMILES: FC(F)(F)c1cc(CN2CCC3(CC2)CC3)cc(Cl)n1] | B | 305.1 |
| Int-67 | 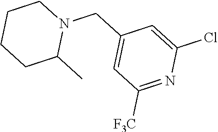 [SMILES: CC1CCCCN1Cc1cc(Cl)nc(C(F)(F)F)c1] | B | 293.0 |
| Int-68 | 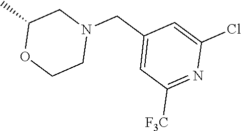 [SMILES: C[C@@H]1CN(Cc2cc(Cl)nc(C(F)(F)F)c2)CCO1] | B | 295.0 |
| Int-69 | 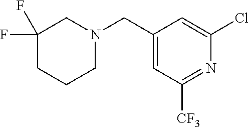 [SMILES: FC1(F)CCCN(Cc2cc(Cl)nc(C(F)(F)F)c2)C1] | B | 315.0 |
| Int-70 | 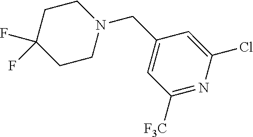 [SMILES: FC1(F)CCN(Cc2cc(Cl)nc(C(F)(F)F)c2)CC1] | B | 315.0 |
| Int-71 | 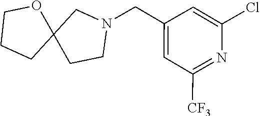 [SMILES: FC(F)(F)c1cc(CN2CCC3(CCCO3)C2)cc(Cl)n1] | A | 321.0 |
| Int-72 | 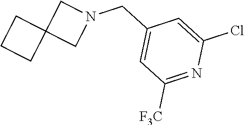 [SMILES: FC(F)(F)c1cc(CN2CC3(CCC3)C2)cc(Cl)n1] | A | 291.0 |
| Int-73 | 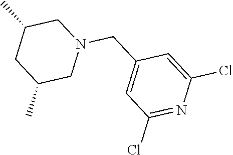 [SMILES: C[C@@H]1C[C@H](C)CN(Cc2cc(Cl)nc(Cl)c2)C1] | B | 273.0 |
| Int-74 | 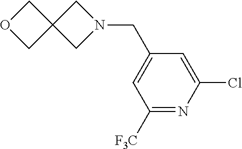 [SMILES: FC(F)(F)c1cc(CN2CC3(COC3)C2)cc(Cl)n1] | B | 293.0 |
| Int-75 | 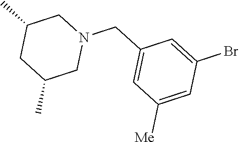 [SMILES: Cc1cc(Br)cc(CN2C[C@H](C)C[C@H](C)C2)c1] | B | 296.1 |
| Int-76 | 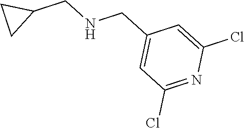 [SMILES: Clc1cc(CNCC2CC2)cc(Cl)n1] | B | 231.0 |
| Int-77 | 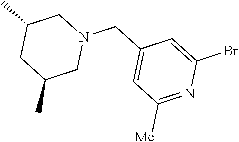 [SMILES: Cc1cc(CN2C[C@@H](C)C[C@H](C)C2)cc(Br)n1] | B | 296.1 |
| Int-78 | 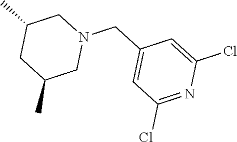 [SMILES: C[C@H]1C[C@H](C)CN(Cc2cc(Cl)nc(Cl)c2)C1] | B | 273.1 |
| Int-79 | 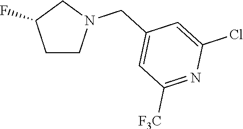 [SMILES: F[C@H]1CCN(Cc2cc(Cl)nc(C(F)(F)F)c2)C1] | A | 283.0 |
| Int-80 | 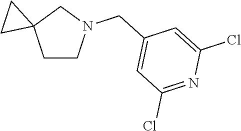 [SMILES: Clc1cc(CN2CCC3(CC3)C2)cc(Cl)n1] | A | 257.0 |
| Int-81 | 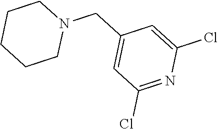 [SMILES: Clc1cc(CN2CCCCC2)cc(Cl)n1] | B | 245.1 |
| Int-82 | 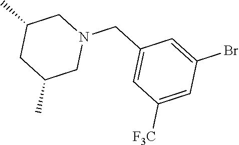 [SMILES: C[C@@H]1C[C@H](C)CN(Cc2cc(Br)cc(C(F)(F)F)c2)C1] | B | 352.0 |
| Int-83 | 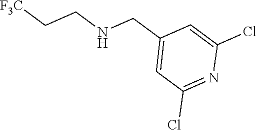 [SMILES: FC(F)(F)CCNCc1cc(Cl)nc(Cl)c1] | B | 273.0 |
| Int-84 | 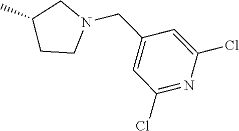 | A | 245.0 |
| Int-85 | 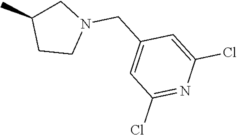 | A | 245.1 |
| Int-86 | 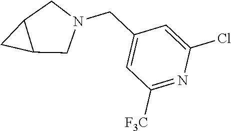 [SMILES: FC(F)(F)c1cc(CN2CC3CC3C2)cc(Cl)n1] | B | 277.0 |
| Int-87 | 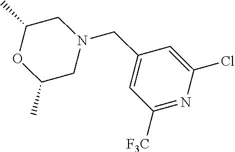 [SMILES: C[C@@H]1CN(Cc2cc(Cl)nc(C(F)(F)F)c2)C[C@H](C)O1] | B | 309.0 |
| Int-88 | 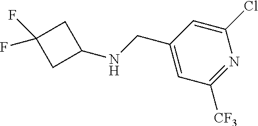 [SMILES: FC1(F)CC(NCc2cc(Cl)nc(C(F)(F)F)c2)C1] | A | 301.0 |
| Int-89 | 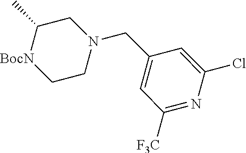 [SMILES: C[C@@H]1CN(Cc2cc(Cl)nc(C(F)(F)F)c2)CCN1C(=O)OC(C)(C)C] | B | 338.1 [M − 56]⁺ |
| Int-90 | 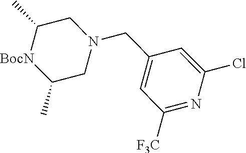 [SMILES: C[C@@H]1CN(Cc2cc(Cl)nc(C(F)(F)F)c2)C[C@H](C)N1C(=O)OC(C)(C)C] | B | 408.1 |
| Int-91 | 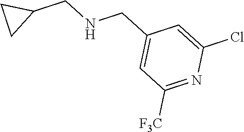 [SMILES: FC(F)(F)c1cc(CNCC2CC2)cc(Cl)n1] | B | 265.0 |
| Int-92 | 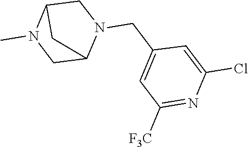 [SMILES: CN1CC2CC1CN2Cc1cc(Cl)nc(C(F)(F)F)c1] | A | 306.1 |

### Intermediate-93. 6-(4-Chloro-2-(4-methyl-4H-1,2,4-triazol-3-yl)phenyl)-isoindolin-1-one

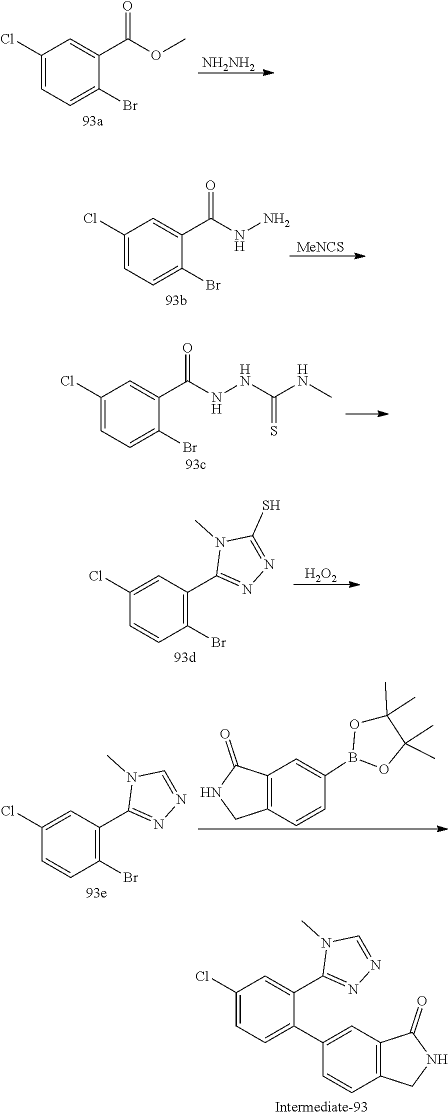

### a) 2-Bromo-5-chlorobenzohydrazide (93b)

To a solution of methyl 2-bromo-5-chlorobenzoate (50.0 g, 200.41 mmol) in ethanol (500.0 mL) was added hydrazine hydrate (50.16 g, 1002.04 mmol) followed by stirring at RT for 12 h. The mixture was concentrated and triturated with pentane to afford the title compound as an off-white solid (49.0 g, 98%). LCMS: 250.9 [M+2+H]

### b) 2-(2-Bromo-5-chlorobenzoyl)-N-methylhydrazine-1-carbothioamide (93c)

To a solution of 2-bromo-5-chlorobenzohydrazide (26 g, 104.21 mmol) in THF (150.0 mL) was added methyl isothiocyanate (11.43 g, 156.32 mmol) followed by stirring at RT for 12 h. The precipitated solid was filtered, washed with cold THF and dried under vacuum to afford the title compound as an off-white solid (28.0 g, 83%). LCMS: 324.0 [M+2+H]⁺.

### c) 5-(2-Bromo-5-chlorophenyl)-4-methyl-4H-1,2,4-triazole-3-thiol (93d)

2-(2-Bromo-5-chlorobenzoyl)-N-methylhydrazine-1-carbothioamide (27.5 g, 85.24 mmol) in 2 N NaOH (137.5 mL) was stirred at 60° C. for 6 h. The mixture was cooled to RT and quenched with ice water. pH was adjusted to ~4 with 1 N HCl. The precipitate was filtered and dried under vacuum to afford the title compound as off-white solid (25.0 g, 96%), LCMS: 305.9 [M+2+H]⁺.

### d) 3-(2-Bromo-5-chlorophenyl)-4-methyl-4H-1,2,4-triazole (93e)

To a solution of 5-(2-bromo-5-chlorophenyl)-4-methyl-4H-1,2,4-triazole-3-thiol (20.0 g, 65.66 mmol) and acetic acid (40 mL) in DCM (200 mL) was added hydrogen peroxide (20 mL) at 0° C. The mixture was slowly warmed to RT and stirred for 1 h. The mixture was concentrated and co-evaporated with toluene twice. The obtained semi-solid was triturated with methanol, filtered, and dried under vacuum to afford the title compound as an off-white solid (16.0 g, 89%), LCMS: 273.9 [M+2+H]⁺.

### e) 6-(4-Chloro-2-(4-methyl-4H-1,2,4-triazol-3-yl) phenyl) isoindolin-1-one (Intermediate-93)

To a degassed solution of 3-(2-bromo-5-chlorophenyl)-4-methyl-4H-1,2,4-triazole (0.5 g, 1.83 mmol), 6-(4,4,5,5-tetramethyl-1,3,2-dioxaborolan-2-yl) isoindolin-1-one (0.713 g, 2.75 mmol), K₂CO₃ (0.76 g, 5.5 mmol) in dioxane (8.0 mL) and water (2.0 mL) was added Pd(amphos)Cl₂ (0.065 g, 0.09 mmol) followed by stirring at 100° C. for 4 h. The mixture was cooled to RT, filtered through celite pad and washed with ethyl acetate. The filtrate was dried over anhydrous sodium sulphate and concentrated. The obtained crude product was purified by silica-gel flash column chromatography using 6-7% MeOH in DCM as eluent to afford the title compound as light brown solid (0.3 g, 50%) LCMS: 325.0 [M+H]⁺.

### Intermediate-94. 6-(2-((4-methyl-4H-1,2,4-triazol-3-yl)methyl)phenyl)isoindo-lin-1-one

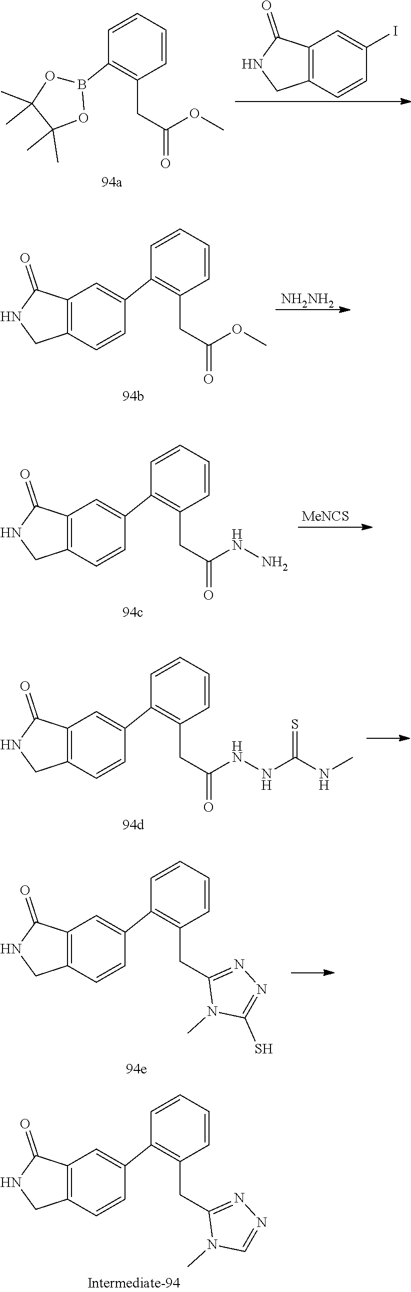

### a) Methyl 2-(2-(3-oxoisoindolin-5-yl) phenyl) acetate (94b)

To a degassed solution of methyl 2-(2-(4,4,5,5-tetramethyl-1,3,2-dioxaborolan-2-yl)phenyl)acetate (0.2 g, 0.724 mmol), 6-iodoisoindolin-1-one (0.19 g, 0.724 mmol), and Cs₂CO₃ (0.47 g, 1.440 mmol) in dioxane (1.3 mL) and water (0.3 mL) was added Pd(dppf)Cl₂ in DCM (0.06 g, 0.070 mmol) followed by stirring at 100° C. for 4 h. The mixture was cooled to RT, filtered through celite pad and washed with ethyl acetate. The filtrate was dried over anhydrous sodium sulphate, concentrated and purified by silica-gel flash column chromatography using 5-6% MeOH in DCM as eluent to afford the title compound as light brown solid (0.065 g, 31%) LCMS: 282.1 [M+H]⁺.

### b) 2-(2-(3-Oxoisoindolin-5-yl)phenyl)acetohydrazide (94c)

The compound was prepared according to the procedure described for Intermediate 93b with appropriate variations in reactants, quantities of reagents, and solvents. LCMS: 282.2 [M+H]⁺.

### c) N-Methyl-2-(2-(2-(3-oxoisoindolin-5-yl)phenyl)acetyl)hydrazine-1-carbothioamide (94d)

The compound was prepared according to the procedure described for Intermediate 93c with appropriate variations in reactants, quantities of reagents, and solvents. LCMS: 355.1 [M+H]⁺.

### d) 6-(2-((5-Mercapto-4-methyl-4H-1,2,4-triazol-3-yl)methyl)phenyl)isoindolin-1-one (94e)

The compound was prepared according to the procedure described for Intermediate 93d with appropriate variations in reactants, quantities of reagents, and solvents. LCMS: 337.1 [M+H]⁺.

### e) 6-(2-((4-Methyl-4H-1,2,4-triazol-3-yl)methyl)phenyl)isoindolin-1-one (Intermediate-94)

The compound was prepared according to the procedure described for Intermediate-93 (last step) with appropriate variations in reactants, quantities of reagents, and solvents. LCMS: 305.1 [M+H]⁺.

The following intermediates were prepared according to the procedure described for Intermediate-93 from the starting materials indicated in the Table with appropriate variations in reactants, quantities of reagents, coupling methods and solvents. The characterization data of the compounds are also summarized in the Table.

| | | LCMS | |
|---|---|---|---|
| No. | Structure | [M + H]⁺ | Starting material |
| Int-95 | 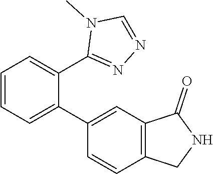 | 580.9 [2M + H]⁺ | 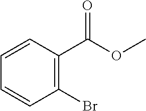 |
| Int-96 | 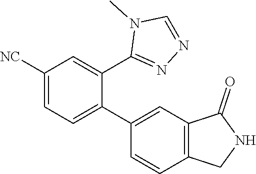 [SMILES: Cn1cnnc1-c1cc(C#N)ccc1-c1ccc2c(c1)C(=O)NC2] | 316.1 | 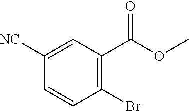 |
| Int-97 | 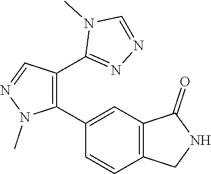 [SMILES: Cn1cnnc1-c1cnn(C)c1-c1ccc2c(c1)C(=O)NC2] | 295.0 | 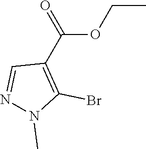 |
| Int-98 | 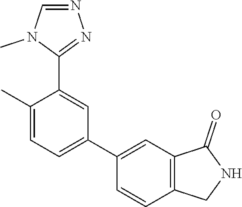 [SMILES: Cc1ccc(-c2ccc3c(c2)C(=O)NC3)cc1-c1nncn1C] | 305.1 | Br |
| Int-99 | 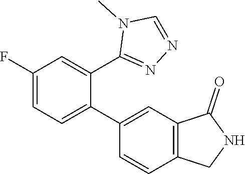 [SMILES: Cn1cnnc1-c1cc(F)ccc1-c1ccc2c(c1)C(=O)NC2] | 309.1 | 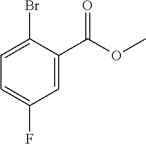 |

### Intermediate-100. 1-(2-Chloro-6-methylpyridin-4-yl)-N-(cyclopropylmethyl)-methanamine

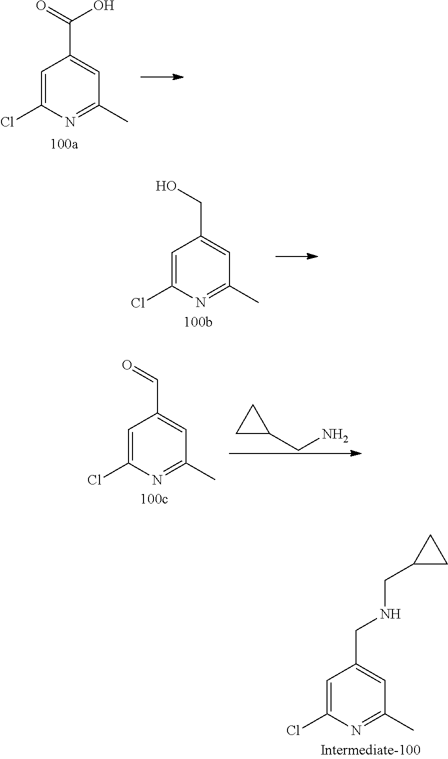

### a) (2-Chloro-6-methylpyridin-4-yl) methanol (100b)

To a solution of 2-chloro-6-methylisonicotinic acid (7.0 g, 40.80 mmol) in THF (70 mL) was added BH₃·THF (81.59 mL, 1 M in THF) dropwise at 0° C. Then the mixture was slowly warmed to RT and stirred for 16 h. The mixture was cooled to 0° C., quenched with methanol (50.0 mL) followed by water (250.0 mL), and extracted with ethyl acetate. The combined organic layer was washed with brine, dried over sodium sulphate, concentrated, and purified by silica-gel flash column chromatography using 15-30% ethyl acetate in hexane as eluent to obtain the title product (5.0 g, 77.10%). LCMS: 158.1 [M+H]⁺.

### b) 2-Chloro-6-methylisonicotinaldehyde (100c)

To a solution of (2-chloro-6-methylpyridin-4-yl) methanol (5.0 g, 31.725 mmol) in DCM (100 mL) was added Dess-Martin periodinane (20.19 g, 47.58 mmol) portion wise at 0° C. Then the mixture was slowly warmed to RT and stirred for 2 h. The mixture was cooled to 0° C., quenched with sodium thiosulphate solution (100 mL) followed by aqueous sodium bicarbonate solution (100 mL), and extracted with DCM. The combined organic layer was dried over sodium sulphate, concentrated, and purified by silica-gel flash column chromatography using 10-30% ethyl acetate in hexane as eluent to obtain the title product (4.0 g, 81%). LCMS: 156.0 [M+H]⁺.

### c) 1-(2-Chloro-6-methylpyridin-4-yl)-N-(cyclopropylmethyl)methanamine (Intermediate-100)

To a solution of 2-chloro-6-methylisonicotinaldehyde (2 g, 12.86 mmol) and cyclopropylmethanamine (2.29 g, 32.14 mmol) in DCM (20 mL) was added catalytic acetic acid at RT followed by stirring for 1 h. STAB (5.45 g, 25.71 mmol) was added into the mixture at 0° C. followed by stirring at RT for 16 h. The mixture was quenched with water and extracted with ethyl acetate. The combined organic layer was dried over anhydrous sodium sulphate, filtered, concentrated, and purified by silica-gel flash column chromatography using 0-30% ethyl acetate in hexane as eluent to afford the title compound (2 g, 73.84%). LCMS: 211.1 [M+H]⁺.

### Intermediate-103. N-(cyclopropylmethyl)-1-(2,6-dichloropyridin-4-yl)ethan-1-amine (Procedure C)

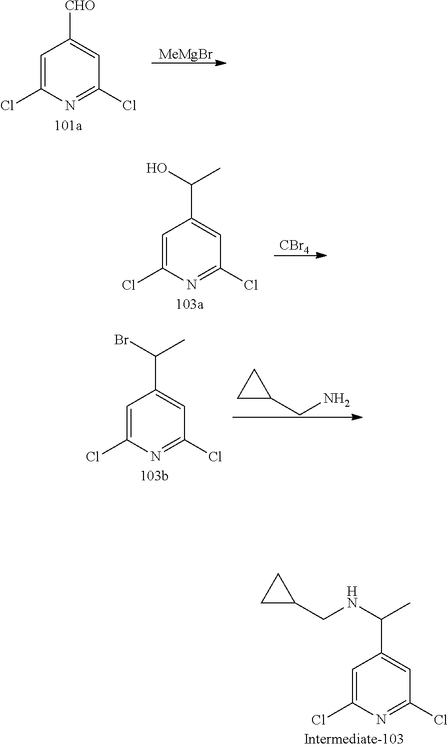

### a) 1-(2,6-Dichloropyridin-4-yl)ethan-1-ol (103a)

To a solution of 2,6-dichloroisonicotinaldehyde (3.0 g, 17.04 mmol) in THF (30 mL) was added methyl magnesium bromide (8.13 g, 68.18 mmol) dropwise at −78° C. followed by stirring for 1 h at the same temperature. The reaction mixture was quenched with saturated ammonium chloride solution and extracted by ethyl acetate. The combined organic layer was dried over anhydrous sodium sulphate, filtered, concentrated, and purified by silica-gel flash column chromatography using 0-5% ethyl acetate in hexane as eluent to afford the title compound (3.5 g, 76.38%) LCMS: 192.0 [M+H]⁺.

### b) 4-(1-Bromoethyl)-2,6-dichloropyridine (103b)

To a solution of 1-(2,6-dichloropyridin-4-yl)ethan-1-ol (0.3 g, 1.56 mmol) in DCM (5 mL) was added triphenylphosphine (0.49 g, 1.87 mmol) and carbon tetra-bromide (0.62 g, 1.87 mmol) at 0° C. followed by stirring at RT for 3 h. The reaction mixture was quenched with water and extracted with DCM. The combined organic layer was dried over anhydrous sodium sulphate, filtered, concentrated, and purified by silica-gel flash column chromatography using 0-5% ethyl acetate in hexane as eluent to afford the title compound (0.23 g, 57.76%). ¹H NMR (CDCl₃, 400 MHz): δ 7.30 (s, 2H), 4.99 (q, 1H), 2.01 (d, 3H).

### c) N-(Cyclopropylmethyl)-1-(2,6-dichloropyridin-4-yl)ethan-1-amine (Intermediate-103)

To a solution of 4-(1-bromoethyl)-2,6-dichloropyridine (0.18 g, 0.70 mmol) and cyclopropylmethanamine (0.06 g, 0.84 mmol) in MeCN (3 mL) was added K₂CO₃ (0.24 g, 1.76 mmol) followed by stirring at RT for 12 h. The mixture was diluted with water and extracted with ethyl acetate. The combined organic layer was dried over anhydrous sodium sulphate, filtered, concentrated, and purified by silica-gel flash column chromatography using 15-30% ethyl acetate in hexane as eluent to afford the title compound (0.19 g). LCMS: 245.0 [M+H]⁺.

### Intermediate-104. 1-(2-Chloro-6-methylpyridin-4-yl)-N-(cyclopropylmethyl)-ethan-1-amine (Procedure D)

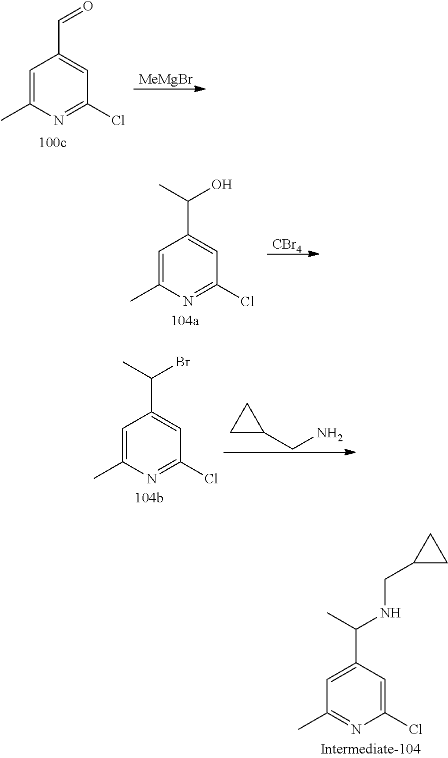

### a) 1-(2-Chloro-6-methylpyridin-4-yl)ethan-1-ol (104a)

To a solution of 2-chloro-6-methylisonicotinaldehyde (2.0 g, 12.855 mmol) in THF (20 mL) was added methyl magnesium bromide (6.13 g, 51.420 mmol) dropwise at −78° C. followed by stirring for 1 h at the same temperature. The reaction mixture was quenched with saturated ammonium chloride solution and extracted by ethyl acetate. The combined organic layer was dried over anhydrous sodium sulphate, filtered, concentrated, and purified by silica-gel flash column chromatography using 0-30% ethyl acetate-hexane as eluent to afford the title compound (1.6 g, 72.5%); LCMS: 172.1 [M+H]⁺.

### b) 4-(1-Bromoethyl)-2-chloro-6-methylpyridine (104b)

To a solution of 1-(2-chloro-6-methylpyridin-4-yl ethan-1-ol (1.60 g, 9.32 mmol) in DCM (50.0 mL) was added triphenylphosphine (2.93 g, 11.18 mmol) and carbon tetrabromide (3.71 g, 11.18 mmol) at 0° C. followed by stirring at RT for 3 h. The reaction mixture was quenched with water and extracted with DCM. The combined organic layer was dried over anhydrous sodium sulphate, filtered, concentrated, and purified by silica-gel flash column chromatography using 0-1% ethyl acetate in hexane as eluent to afford the title compound (1.4 g, 64.03%); LCMS: 233.9 [M+H]⁺.

### c) 1-(2-Chloro-6-methylpyridin-4-yl)-N-(cyclopropylmethyl) ethan-1-amine (Intermediate-104)

To a solution 4-(1-bromoethyl)-2-chloro-6-methylpyridine (0.50 g, 2.13 mmol) and cyclopropylmethanamine (0.30 g, 4.26 mmol) in MeCN (5.0 mL) was added K₂CO₃ (0.74 g, 5.33 mmol) followed by stirring at RT for 12 h. The reaction mixture was quenched with water and extracted with DCM. The combined organic layer was dried over anhydrous sodium sulphate, filtered, concentrated, and purified by silica-gel flash column chromatography using 15-30% ethyl acetate in hexane as eluent to afford the title compound (0.450 g, 93.92%) LCMS: 225.1 [M+H]⁺.

The following intermediates were prepared according to the procedure described for Intermediate-76 (procedure A) or Intermediate-18 (procedure B) from the starting materials indicated in the Table with appropriate variations in reactants, quantities of reagents, coupling methods and solvents. The characterization data of the compounds are also summarized in the Table.

| | | Pro- | LCMS |
|---|---|---|---|
| No. | Structure | cedure | [M + H]⁺ |
| Int-106 | 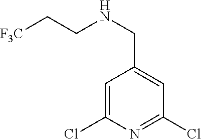 [SMILES: FC(F)(F)CCNCc1cc(Cl)nc(Cl)c1] | B | 273.2 |
| Int-107 | 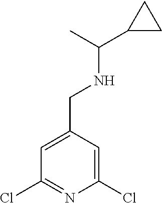 | B | 245.1 |
| Int-108 | 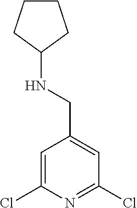 [SMILES: Clc1cc(CNC2CCCC2)cc(Cl)n1] | A | 245.1 |
| Int-109 | 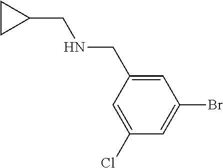 [SMILES: Clc1cc(Br)cc(CNCC2CC2)c1] | A | 274.0 |
| Int-110 | 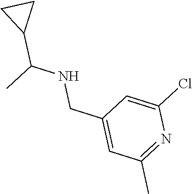 | B | 225.1 |
| Int-111 | 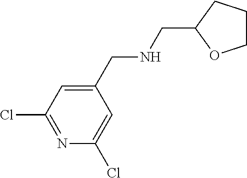 [SMILES: Clc1cc(CNCC2CCCO2)cc(Cl)n1] | A | 261.1 |

The following intermediates were prepared according to the procedure described for Intermediate-103 (procedure C) or Intermediate-104 (procedure D) from the starting materials indicated in the Table with appropriate variations in reactants, quantities of reagents, coupling methods and solvents. The characterization data of the compounds are also summarized in the Table.

| No. | Structure | Procedure | LCMS [M + H]⁺ |
|---|---|---|---|
| Int-112 | 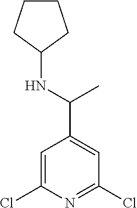 [SMILES: CC(NC1CCCC1)c1cc(Cl)nc(Cl)c1] | C | 259.1 |
| Int-113 | 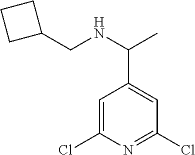 [SMILES: CC(NCC1CCC1)c1cc(Cl)nc(Cl)c1] | C | 259.1 |
| Int-114 | 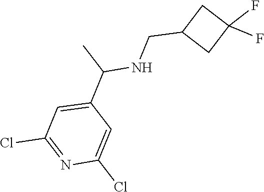 [SMILES: CC(NCC1CC(F)(F)C1)c1cc(Cl)nc(Cl)c1] | C | 295.0 |
| Int-115 | 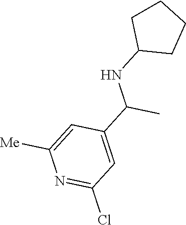 [SMILES: Cc1cc(C(C)NC2CCCC2)cc(Cl)n1] | D | 239.1 |

The following intermediates were prepared according to the procedure described for Intermediate-1 from the starting materials indicated in the Table with appropriate variations in reactants, quantities of reagents, coupling methods and solvents. The characterization data of the compounds are also summarized in the Table.

| | | LCMS | |
|---|---|---|---|
| No. | Structure | [M + H]⁺ | Starting material |
| Int-119 | 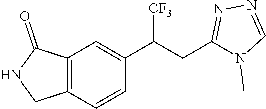 [SMILES: Cn1cnnc1CC(c1ccc2c(c1)C(=O)NC2)C(F)(F)F] | 311.1 | F3C OEt |
| Int-120 | 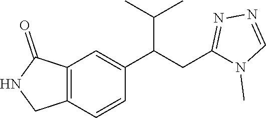 [SMILES: CC(C)C(Cc1nncn1C)c1ccc2c(c1)C(=O)NC2] | 285.0 | OEt |
| Int-121 | 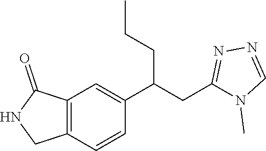 [SMILES: CCCC(Cc1nncn1C)c1ccc2c(c1)C(=O)NC2] | 285.2 | OEt |
| Int-122 | 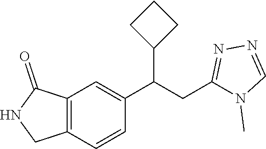 [SMILES: Cn1cnnc1CC(c1ccc2c(c1)C(=O)NC2)C1CCC1] | 297.1 | OEt |
| Int-123 | 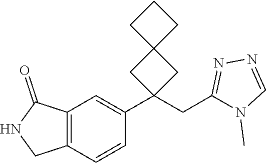 [SMILES: Cn1cnnc1CC1(c2ccc3c(c2)C(=O)NC3)CC2(CCC2)C1] | 323.2 | OEt |

### Intermediate 124. 6-(3,3-Difluoro-1-((4-methyl-4H-1,2,4-triazol-3-yl)methyl)-cyclobutyl)isoindolin-1-one

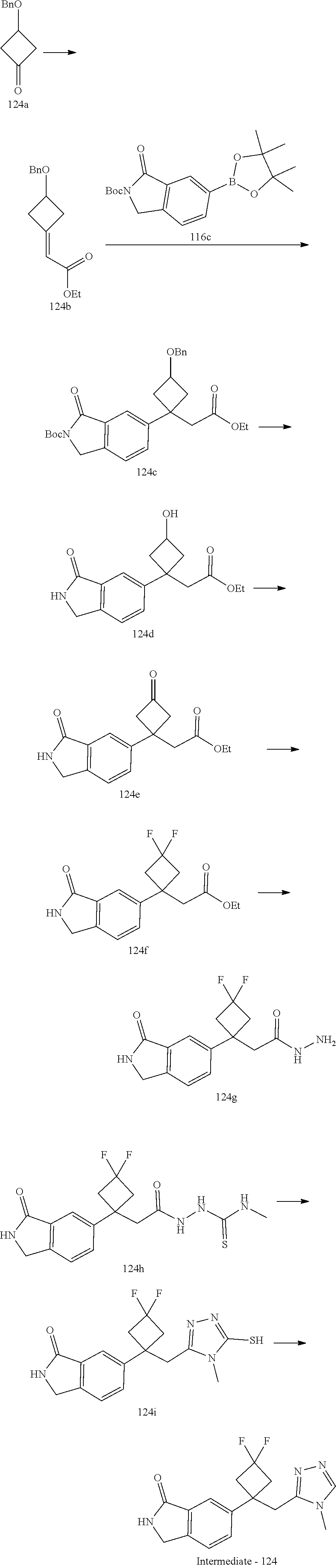

### a) Ethyl 2-(3-(benzyloxy)cyclobutylidene) acetate (124b)

To a stirred solution of 3-(benzyloxy) cyclobutan-1-one (25.0 g, 141.87 mmol) in DCM, was added ethyl (triphenylphosphoranylidene)acetate (64.25 g, 184.43 mmol) portionwise at RT and stirred for 20 h. The mixture was concentrated, the crude mixture was triturated with hexane and filtered. The organic layer was evaporated and purified by silica-gel flash column chromatography using 0-5% ethyl acetate in hexane as eluent to afford the title compound (21.5 g, 61.53%), LCMS: 247.2 [M+H]⁺.

### b) tert-Butyl 6-(3-(benzyloxy)-1-(2-ethoxy-2-oxoethyl) cyclobutyl)-1-oxoiso-indoline-2-carboxylate (124c)

To a degassed solution of tert-butyl 1-oxo-6-(4,4,5,5-tetramethyl-1,3,2-dioxa-borolan-2-yl)isoindoline-2-carboxylate (29.17 g, 81.20 mmol), compound 124b (10.00 g, 40.60 mmol) and N, N-diethylethanamine (8.22 g, 81.19 mmol) in 1,4-dioxane (110.00 mL) and water (66.0 mL) was added [Rh(COD)Cl]₂ (1.001 g, 2.03 mmol) followed by stirring for 3 h at RT. The mixture was quenched with water and extracted with ethyl acetate. The combined organic layer was treated as in example Intermediate-1 step c using 15-25% ethyl acetate in hexane as eluent to afford the title compound (8.0 g, 41.08%). ¹HNMR: NMR (400 MHz, CDCl₃): δ 7.7 (d, 1H), 7.50-7.43 (m, 1H), 7.41-7.28 (m, 6H), 4.74 (s, 2H), 4.45 (s, 2H), 4.31-4.23 (m, 1H), 3.99-3.90 (m, 2H), 2.94-2.87 (m, 2H), 2.72 (s, 2H), 2.43-2.38 (m, 2H), 1.26 (s, 9H), 1.09 (t, 3H).

### c) Ethyl 2-(3-hydroxy-1-(3-oxoisoindolin-5-yl) cyclobutyl) acetate (124d)

To a solution of compound 124c (8.0 g, 16.68 mmol) in DCM (400.0 mL) was added concentrated boron tribromide (16.72 g, 66.72 mmol) at −78° C. The mixture was slowly warm to RT and stirred for 3 h. The mixture was poured into ice cold water and diluted with aqueous sodium bicarbonate solution and extracted with DCM. The combined organic layer was as in example Int-1 step c using 0-10% MeOH in DCM as eluent to afford the title compound (3.5 g, 72.52%). LCMS: 290.1 [M+H]⁺.

### d) Ethyl 2-(3-oxo-1-(3-oxoisoindolin-5-yl) cyclobutyl) acetate (124e)

To a solution of compound 124d (1.5 g, 5.18 mmol) in DCM (20.0 mL) was added Dess-Martin periodinane (3.30 g, 7.77 mmol) at 0° C. The mixture was slowly warmed to RT and stirred for 5 h. The reaction was quenched with aqueous sodium thiosulphate solution followed by addition of aqueous sodium hydrogen carbonate solution. The mixture was stirred for 10 min and extracted with ethyl acetate. The combined organic layer was treated as in example Int-1 step c using 0-5% methanol in DCM as eluent to afford the title compound (1.20 g, 80.57%). LCMS: 288.2 [M+H]⁺.

### e) Ethyl 2-(3,3-difluoro-1-(3-oxoisoindolin-5-yl) cyclobutyl) acetate (124f)

To a solution of compound 124e (2.8 g, 9.74 mmol) in DCM (100.0 mL) was added diethylaminosulfur trifluoride (9.42 g, 58.47 mmol) at −40° C. Then mixture was slowly warmed to RT and stirred for 6 h. The mixture was poured into ice cold water and diluted with aqueous sodium bicarbonate solution and extracted with DCM. The combined organic layer was treated as in example Int-124 step d to afford the title compound (2.0 g, 66.36%). LCMS: 310.1 [M+H]⁺

### f) 2-(3,3-Difluoro-1-(3-oxoisoindolin-5-yl) cyclobutyl)acetohydrazide (124g)

The compound was prepared according to the procedure described for Intermediate-if with appropriate variations in reactants, quantities of reagents, and solvents. LCMS: 296.1 [M+H]⁺.

### g) 2-(2-(3,3-Difluoro-1-(3-oxoisoindolin-5-yl) cyclobutyl) acetyl)-N-methyl-hydrazine-1-carbothioamide (124h)

The compound was prepared according to the procedure described for Intermediate-1g with appropriate variations in reactants, quantities of reagents, and solvents. LCMS: 369.1 [M+H]⁺.

### h) 6-(3,3-Difluoro-1-((5-mercapto-4-methyl-4H-1,2,4-triazol-3-yl) methyl) cyclobutyl) isoindolin-1-one (124i)

The compound was prepared according to the procedure described for Intermediate-1h with appropriate variations in reactants, quantities of reagents, and solvents. LCMS: 351.1 [M+H]⁺.

### i) 6-(3,3-Difluoro-1-((4-methyl-4H-1,2,4-triazol-3-yl)methyl)cyclobutyl)-isoindolin-1-one (Intermediate-124)

The compound was prepared according to the procedure described for Intermediate-1 with appropriate variations in reactants, quantities of reagents, and solvents. LCMS: 351.1 [M+H]⁺.

### Intermediate 125. 6-(3-Methoxy-1-((4-methyl-4H-1,2,4-triazol-3-yl)methyl)-cyclobutyl)isoindolin-1-one

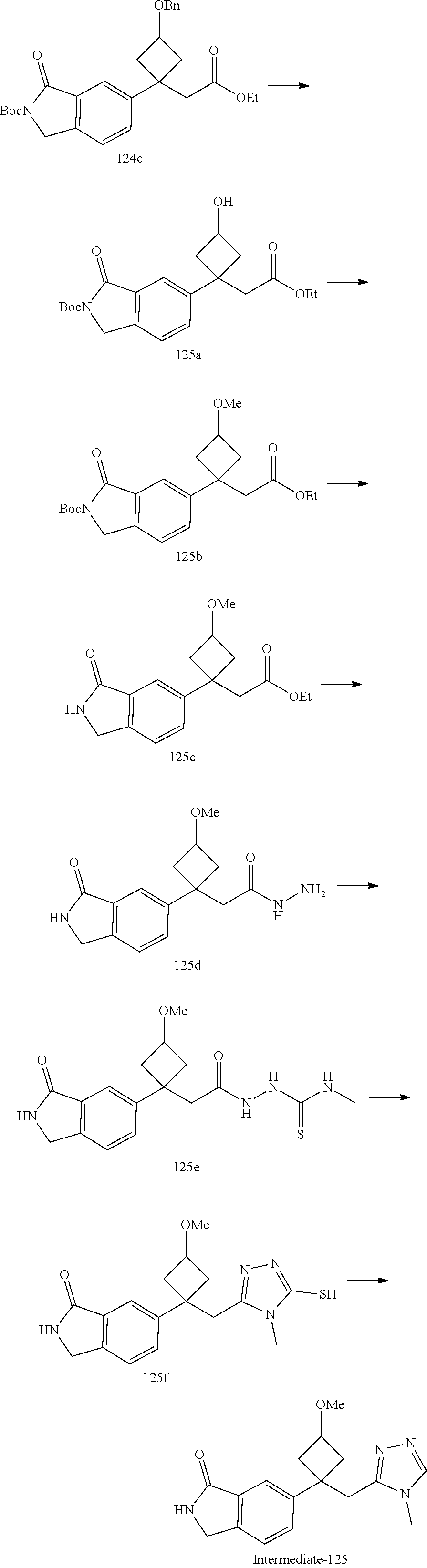

### a) tert-Butyl 6-(1-(2-ethoxy-2-oxoethyl)-3-hydroxycyclobutyl)-1-oxoiso-indoline-2-carboxylate (125a)

To a solution of tert-butyl 6-(3-(benzyloxy)-1-(2-ethoxy-2-oxoethyl) cyclo-butyl)-1-oxoisoindoline-2-carboxylate (5.9 g, 12.30 mmol) in ethanol (250.0 mL) was added moist palladium on activated charcoal (0.66 g, 6.15 mmol) at RT followed by stirring for 12 h at RT. The mixture was filtered through celite, concentrated, and purified by silica-gel flash column chromatography using 0-15% MeOH in DCM as eluent to afford the title compound (4.5 g, 93.92%). LCMS: 290.1 [M−100+H]⁺

### b) tert-Butyl 6-(1-(2-ethoxy-2-oxoethyl)-3-methoxycyclobutyl)-1-oxoiso-indoline-2-carboxylate (125b)

To a solution of compound 125a (3.00 g, 7.70 mmol) in dry THF (30.0 mL) was added sodium hydride (0.22 g, 9.24 mmol) at 0° C. followed by stirring for 30 min. Iodomethane (1.64 g, 11.55 mmol) was added to the mixture and stirred at 0° C. for 1 h. The mixture was quenched with aqueous NH₄Cl solution and extracted with ethyl acetate. The combined organic layer was treated as in example Intermediate-1 step c using 0-60% ethyl acetate in hexane as eluent to afford the title compound (1.1 g, 35.39%). LCMS: 404.3 [M+H]⁺.

### c) Ethyl 2-(3-methoxy-1-(3-oxoisoindolin-5-yl)cyclobutyl)acetate (125c)

The compound was prepared according to the procedure described for Intermediate-1e with appropriate variations in reactants, quantities of reagents, and solvents. LCMS: 304.4 [M+H]⁺.

### d) 2-(3-Methoxy-1-(3-oxoisoindolin-5-yl)cyclobutyl)acetohydrazide (125d)

The compound was prepared according to the procedure described for Intermediate-if with appropriate variations in reactants, quantities of reagents, and solvents. LCMS: 290.1 [M+H]⁺.

### e) 2-(2-(3-Methoxy-1-(3-oxoisoindolin-5-yl) cyclobutyl) acetyl)-N-methyl-hydrazine-1-carbothioamide (125e)

The compound was prepared according to the procedure described for Intermediate-1g with appropriate variations in reactants, quantities of reagents, and solvents. LCMS: 363.05 [M+H]⁺.

### f) 6-(1-((5-Mercapto-4-methyl-4H-1,2,4-triazol-3-yl) methyl)-3-methoxycyclo-butyl) isoindolin-1-one (125f)

The compound was prepared according to the procedure described for Inter-mediate-1h with appropriate variations in reactants, quantities of reagents, and solvents. LCMS: 345.1 [M+H]⁺.

### h) 6-(3-Methoxy-1-((4-methyl-4H-1,2,4-triazol-3-yl)methyl)cyclobutyl)-isoindolin-1-one (Intermediate-125)

The compound was prepared according to the procedure described for Intermediate-1 (last step) with appropriate variations in reactants, quantities of reagents, and solvents. LCMS: 313.2 [M+H]⁺.

### Intermediate 127. 6-(2-(4-Methyl-4H-1,2,4-triazol-3-yl)ethyl)isoindolin-1-one

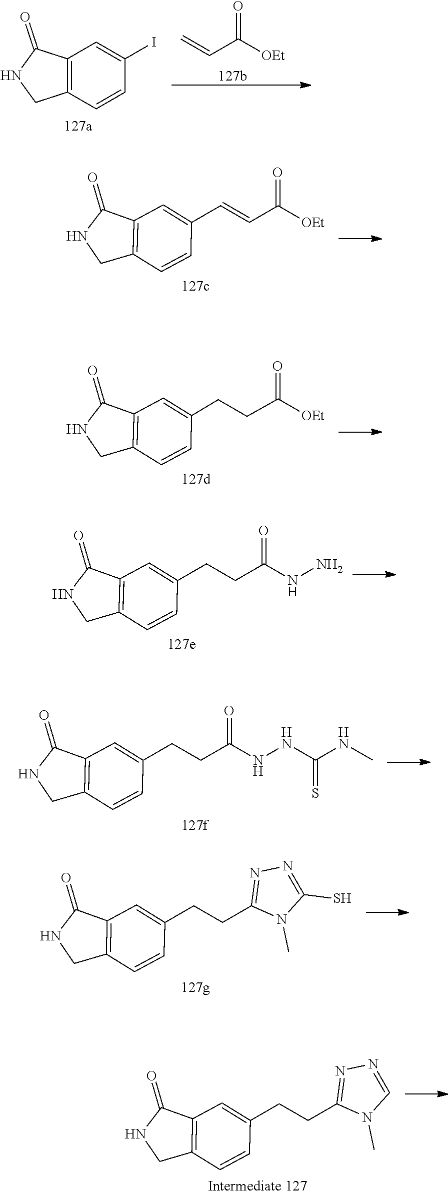

### a) Ethyl (E)-3-(3-oxoisoindolin-5-yl)acrylate (127c)

To a degassed solution of 6-iodoisoindolin-1-one (3.8 g, 14.67 mmol) triethyl-amine and ethyl acrylate (4.406 g, 44.0 mmol) in DMF (35 ml) was added palladium acetate (0.17 g, 0.73 mmol) and tris(2-methylphenyl)phosphane (0.45 g, 1.46 mmol) followed by stirring at 90° C. for 16 h. The mixture was diluted with water and extracted with EtOAc. The combined organic layer was treated as in example Intermediate-1 step c using 80% ethyl acetate in hexane as eluent to afford the title compound (0.9 g, 26.53%), LCMS: 232.1 [M+H]⁺.

### b) Ethyl 3-(3-oxoisoindolin-5-yl)propanoate (127d)

To a stirred solution of compound 127c (0.9 g, 3.89 mmol) in EtOAc (100 ml) was added Pd on carbon and the mixture was stirred at RT in Parr Shaker under hydrogen (80 psi) for 12 h. The mixture was filtered through celite and concentrated to afford title compound (0.8 g, 88.2%), LCMS: 234.1 [M+H]⁺.

### c) 3-(3-Oxoisoindolin-5-yl)propanehydrazide (127e)

The compound was prepared according to the procedure described for Intermediate-if with appropriate variations in reactants, quantities of reagents, and solvents. LCMS: 220.1 [M+H]⁺.

### d) N-Methyl-2-(3-(3-oxoisoindolin-5-yl)propanoyl)hydrazine-1-carbothioamide (127f)

The compound was prepared according to the procedure described for Inter-mediate-1g with appropriate variations in reactants, quantities of reagents, and solvents. LCMS: 293.0 [M+H]⁺.

### e) 6-(2-(5-Mercapto-4-methyl-4H-1,2,4-triazol-3-yl)ethyl)isoindolin-1-one (127g)

The compound was prepared according to the procedure described for Inter-mediate-1h with appropriate variations in reactants, quantities of reagents, and solvents. LCMS: 275.1 [M+H]⁺.

### f) 6-(2-(4-Methyl-4H-1,2,4-triazol-3-yl)ethyl)isoindolin-1-one (Intermediate-127)

The compound was prepared according to the procedure described for Intermediate-1 (last step) with appropriate variations in reactants, quantities of reagents, and solvents. LCMS: 243.1 [M+H]⁺.

### Intermediate 128. 6-((1-(4-Methyl-4H-1,2,4-triazol-3-yl)propan-2-yl)amino)-isoindolin-1-one

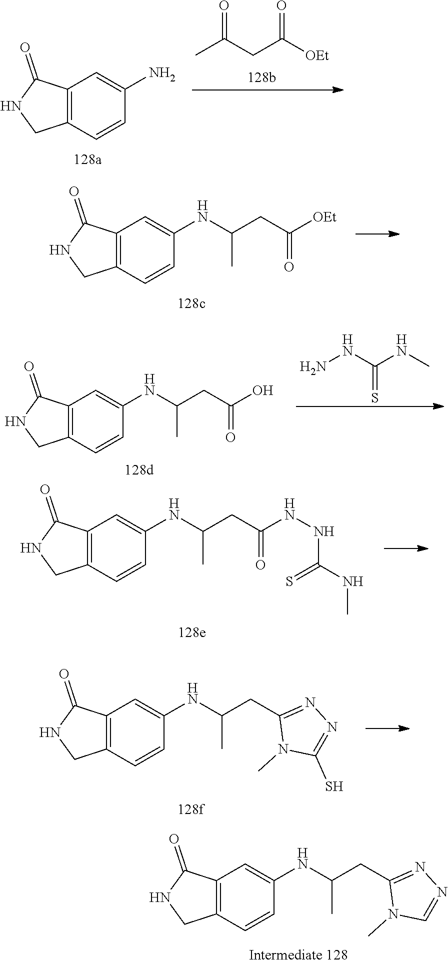

### a) Ethyl 3-((3-oxoisoindolin-5-yl)amino)butanoate (128c)

To a stirred solution of ethyl 3-oxobutanoate (2.50 g, 19.1 mmol) and 6-amino-isoindolin-1-one (2.85 g, 19.21 mmol) in 1,2-dichloroethene was added 2.5 ml of acetic acid followed by stirring at RT for 20 min. Then STAB (8.14 g, 38.42 mmol) was added to the mixture at 0° C. portion wise. The mixture was slowly warmed to RT and stirred for 12 h. The reaction mixture was quenched with water and extracted with DCM. The combined organic layer was dried over anhydrous sodium sulphate, filtered, concentrated to afford the title compound (4.6 g). LC-MS: 263.1 [M+H]⁺.

### b) 3-((3-oxoisoindolin-5-yl)amino)butanoic acid (128d)

The compound was prepared according to the procedure described for Inter-mediate-23e with appropriate variations in reactants, quantities of reagents, and solvents. LCMS: 235.1[M+H]⁺.

### c) N-Methyl-2-(3-((3-oxoisoindolin-5-yl)amino)butanoyl)hydrazine-1-carbo-thioamide (128e)

To a stirred solution of compound 128d (1.0 g, 4.27 mmol) and N-methyl-hydrazinecarbothioamide (0.67 g, 6.4 mmol) in acetonitrile (15.0 mL) was added 1-methylimidazole (1.75 g, 21.34 mmol) followed by stirring for 10 min. Then chloro-dimethylaminomethylidene)-dimethylazanium (1.74 g, 12.80 mmol) was added to the mixture followed by stirring at RT for 12 h. The mixture was concentrated and purified by silica-gel flash column chromatography using 15% methanol in DCM as eluent to afford the title compound (0.90 g). LCMS: 322.1 [M+H]⁺.

### d) 6-((1-(5-Mercapto-4-methyl-4H-1,2,4-triazol-3-yl)propan-2-yl)amino)iso-indolin-1-one (128f)

The compound was prepared according to the procedure described for Intermediate-1h with appropriate variations in reactants, quantities of reagents, and solvents. LCMS: 304.1 [M+H]⁺.

### e) 6-((1-(4-Methyl-4H-1,2,4-triazol-3-yl)propan-2-yl)amino)isoindolin-1-one (Intermediate-128)

The compound was prepared according to the procedure described for Intermediate-1 (last step) with appropriate variations in reactants, quantities of reagents, and solvents. LCMS: 272.2 [M+H]⁺.

The following intermediates were prepared according to the procedure described for Intermediate-93 from the starting materials indicated in the Table with appropriate variations in reactants, quantities of reagents, coupling methods and solvents. The characterization data of the compounds are also summarized in the Table.

| No. | Structure | LCMS [M + H]⁺ | Starting material |
|---|---|---|---|
| Int-131 | 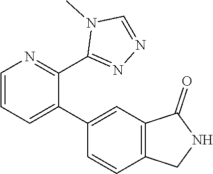 [SMILES: Cn1cnnc1-c1ncccc1-c1ccc2c(c1)C(=O)NC2] | 292.2 | 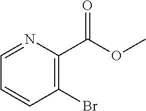 |
| Int-132 | 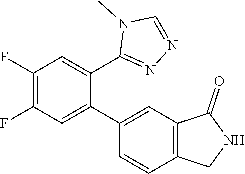 [SMILES: Cn1cnnc1-c1cc(F)c(F)cc1-c1ccc2c(c1)C(=O)NC2] | 327.1 | 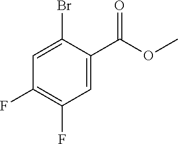 |
| Int-133 | 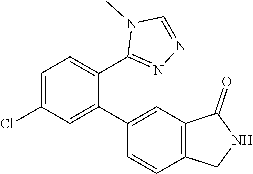 [SMILES: Cn1cnnc1-c1ccc(Cl)cc1-c1ccc2c(c1)C(=O)NC2] | 325.1 | 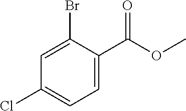 |
| Int-134 | 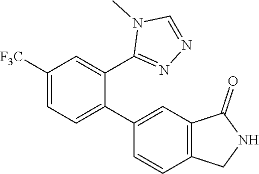 [SMILES: Cn1cnnc1-c1cc(C(F)(F)F)ccc1-c1ccc2c(c1)C(=O)NC2] | 359.1 | 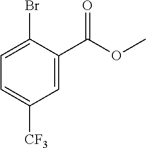 |
| Int-135 | 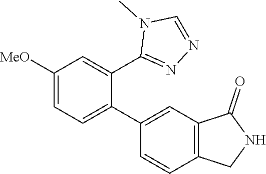 [SMILES: COc1ccc(-c2ccc3c(c2)C(=O)NC3)c(-c2nncn2C)c1] | 321.1 | 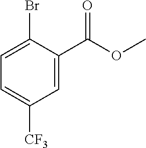 |
| Int-136 | 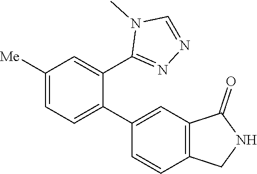 [SMILES: Cc1ccc(-c2ccc3c(c2)C(=O)NC3)c(-c2nncn2C)c1] | 305.1 | 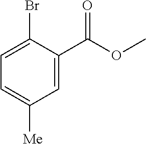 |
| Int-137 | 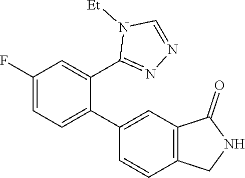 [SMILES: CCn1cnnc1-c1cc(F)ccc1-c1ccc2c(c1)C(=O)NC2] | 323.1 | 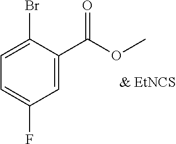 |
| Int-138 | 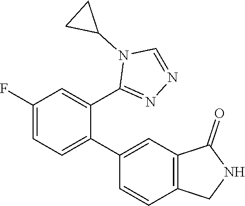 [SMILES: O=C1NCc2ccc(-c3ccc(F)cc3-c3nncn3C3CC3)cc21] | 335.1 | 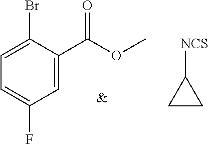 |

### Intermediate 139. 6-(3,4-Difluoro-2-(4-methyl-4H-1,2,4-triazol-3-yl)phenyl)-isoindolin-1-one

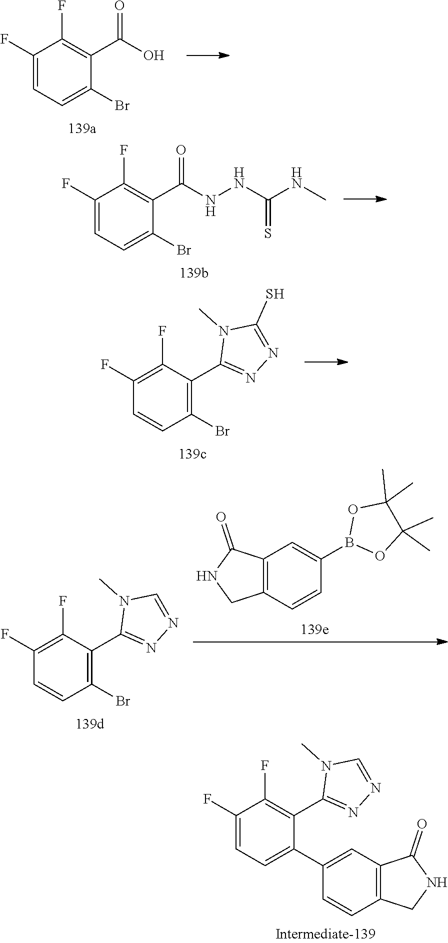

### a) 2-(2-Bromo-5-fluorophenyl)-4,5-dihydro-1H-imidazole (139b)

To a solution of 6-bromo-2,3-difluorobenzoic acid (4.0 g, 16.88 mmol), N-methylhydrazine carbothioamide (2.13 g, 20.54 mmol) and DIPEA (4.36 g, 33.75 mmol) in DMF (30.0 mL) was added HATU (4.77 g, 20.25 mmol) followed by stirring at RT for 12 h. The mixture was concentrated and ice added to afford solid precipitate. Then solid was filtered and triturated with pentane and MTBE and purified by silica-gel flash column chromatography using 15% MeOH in DCM as eluent to afford the title compound as off white solid (2.15 g, 39.30%). LCMS: 323.9 [M+H]⁺.

### b) 5-(6-Bromo-2,3-difluorophenyl)-4-methyl-4H-1,2,4-triazole-3-thiol (139c)

The compound was prepared according to the procedure described for Intermediate-93d with appropriate variations in reactants, quantities of reagents, and solvents. LCMS: 305.9 [M+H]⁺.

### c) 3-(6-Bromo-2,3-difluorophenyl)-4-methyl-4H-1,2,4-triazole (139d)

The compound was prepared according to the procedure described for Intermediate-93e with appropriate variations in reactants, quantities of reagents, and solvents. LCMS: 274.0 [M+H]⁺.

### d) 6-(3,4-Difluoro-2-(4-methyl-4H-1,2,4-triazol-3-yl)phenyl)isoindolin-1-one (Intermediate-139)

The compound was prepared according to the procedure described for Intermediate-93 with appropriate variations in reactants, quantities of reagents, and solvents. LCMS: 327.0 [M+H]⁺.

The following intermediates were prepared according to the procedure described for Intermediate-139 from the starting materials indicated in the Table with appropriate variations in reactants, quantities of reagents, coupling methods and solvents. The characterization data of the compounds are also summarized in the Table.

| | | LCMS | |
|---|---|---|---|
| No. | Structure | [M + H]⁺ | Starting material |
| Int-140 | 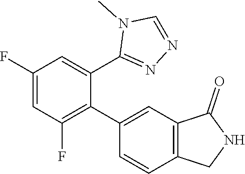 [SMILES: Cn1cnnc1-c1cc(F)cc(F)c1-c1ccc2c(c1)C(=O)NC2] | 327.1 | 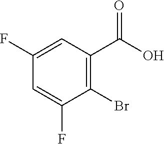 [SMILES: O=C(O)c1cc(F)cc(F)c1Br] |
| Int-141 | 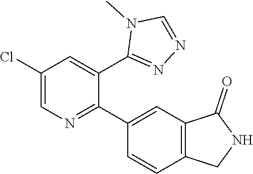 [SMILES: Cn1cnnc1-c1cc(Cl)cnc1-c1ccc2c(c1)C(=O)NC2] | 326.1 | 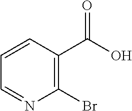 |

### Intermediate-142. 6-(4-Fluoro-2-(4-methyl-4H-1,2,4-triazol-3-yl)phenyl)-4-(trifluoromethyl)isoindolin-1-one

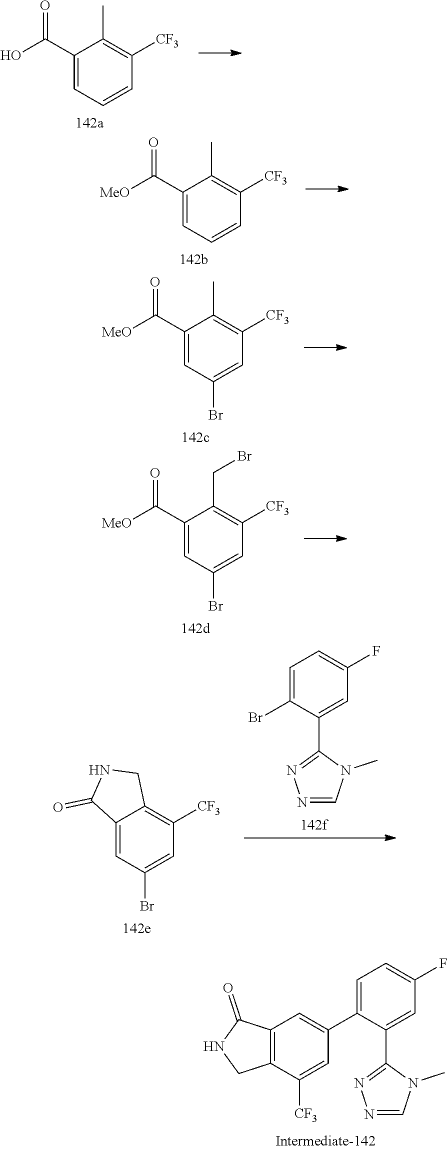

### a) Methyl 2-methyl-3-(trifluoromethyl)benzoate (142b)

To a stirred suspension of 2-methyl-3-(trifluoromethyl) benzoic acid (4.0 g, 19.593 mmol) in MeOH (24.0 mL) was added dropwise concentrated H₂SO₄ (1.922 g, 19.59 mmol) at 0° C. followed by stirring at 80° C. overnight. The reaction mixture was quenched with aqueous NaHCO₃ solution and extracted with hexane. The combined organic layer was dried over anhydrous sodium sulphate, filtered, and concentrated to afford the title compound (3.4 g) as crude product which was used in next step as such. LCMS: 218.9 [M+H]⁺.

### b) Methyl 5-bromo-2-methyl-3-(trifluoromethyl)benzoate (142c)

To a stirred solution of compound 142b (3.4 g, 15.58 mmol) in acetic acid (35.0 mL) and nitric acid (10.41 g, 165.18 mmol) was added bromine (2.86 g, 35.84) dropwise at 0° C. Then silver nitrate (3.63 g, 21.34 mmol) dissolved in 8.0 mL of water was added dropwise followed by stirring for 12 h at RT. The mixture was filtered through filter paper. The filtrate was diluted with water and extracted with ethyl acetate. The combined organic layer was treated as in example Intermediate-1 step c using 0-5% ethyl acetate in hexane as eluent to obtain the desired product (3.2 g, 69.13%). ¹H-NMR (400 MHz, CDCl₃): δ 8.08 (d, 1H), 7.91 (d, 1H), 3.95 (s, 3H), 2.61 (s, 3H).

### c) Methyl 5-bromo-2-(bromomethyl)-3-(trifluoromethyl)benzoate (142d)

To a solution of compound 142c (3.2 g, 10.77 mmol) in CCl₄ (32.0 mL) was added N-bromosuccinimide (2.88 g, 16.15 mmol), followed by benzoyl peroxide (0.26 g, 1.07 mmol) and allowed the mixture to stir at 80° C. for 12 h in dark. The mixture was concentrated. The crude product was diluted with water and extracted with hexane. The combined organic layer was treated as in example Intermediate-1 step c using 0-2% ethyl acetate in hexane as eluent to obtain the desired product (3.0 g, 74.08%). LCMS: 375.3 [M+H]⁺.

### d) 6-Bromo-4-(trifluoromethyl) isoindolin-1-one (142e)

To a solution of compound 142d (3.0 g, 7.98 mmol) in DCM (30.0 mL) was added 7 M methanol-ammonia solution (15.0 mL) at 0° C. followed by stirring at RT for 16 h. The mixture was concentrated and the crude product diluted with water and extracted with ethyl acetate. The combined organic layer was treated as in example Intermediate-1 step c using 30-50% ethyl acetate in hexane as eluent to obtain the desired product (2.2 g, 98.46%). LCMS: 280.0 [M+H]⁺.

### e) 6-(4-Fluoro-2-(4-methyl-4H-1,2,4-triazol-3-yl)phenyl)-4-(trifluoromethyl)iso-indolin-1-one (Intermediate-142)

To a degassed solution of compound 142e (0.05 g, 0.18 mmol), 3-(2-bromo-5-fluorophenyl)-4-methyl-4H-1,2,4-triazole (0.06 g, 0.21 mol), bis(pinacolato)diborane (0.07 g, 0.26 mmol), and CsF (0.13 g, 0.85 mmol) in MeOH was added a mixture of cataCXium-A (0.016 g, 0.04 mmol), and Pd(OAc)2 (0.008 g, 0.03 mmol) in toluene at 50° C. The mixture was heated at 80° C. for 12 h. The mixture was filtered through celite and washed with ethyl acetate. The filtrate was diluted with water and extracted with ethyl acetate. The combined organic layer was treated as in example Intermediate-124 step d using 0 to 7% MeOH in DCM as eluent to afford the title compound as yellow solid (0.017 g, 25%). LCMS: 377.1 [M+H]⁺.

The following intermediates were prepared according to the procedure described for Intermediate-142 from the starting materials indicated in the Table with appropriate variations in reactants, quantities of reagents, coupling methods and solvents. The characterization data of the compounds are also summarized in the Table.

| | | LCMS | |
|---|---|---|---|
| No. | Structure | [M + H]⁺ | Starting material |
| Int-143 | 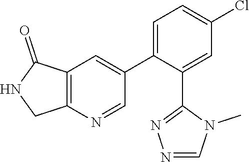 [SMILES: Cn1cnnc1-c1cc(Cl)ccc1-c1cnc2c(c1)C(=O)NC2] | 326.1 | 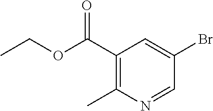 |
| Int-144 | 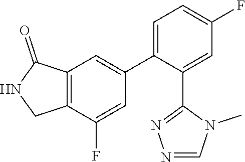 [SMILES: Cn1cnnc1-c1cc(F)ccc1-c1cc(F)c2c(c1)C(=O)NC2] | 327.1 | 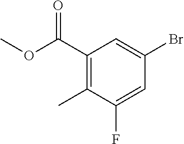 |
| Int-145 | 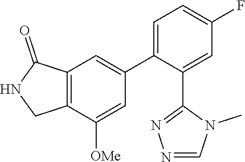 [SMILES: COc1cc(-c2ccc(F)cc2-c2nncn2C)cc2c1CNC2=O] | 339.1 | 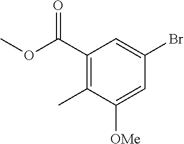 |

### Intermediate-146. 6-(4-Chloro-2-(1H-pyrazol-3-yl)phenyl)isoindolin-1-one

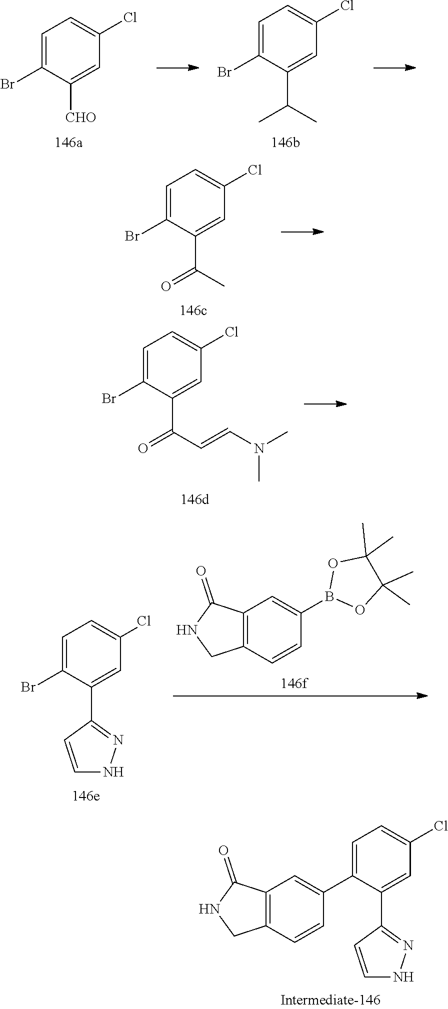

### a) 1-(2-Bromo-5-chlorophenyl) ethan-1-ol (146b)

To a solution 2-bromo-5-chlorobenzaldehyde (1.0 g, 4.56 mmol) in THF (15.0 mL) was added methyl magnesium bromide (6.07 mL, 18.21 mmol, 3.0 M in diethyl ether) at −78° C. followed by stirring at −78° C. for 1h. The reaction mixture was quenched by saturated ammonium chloride solution and extracted with ethyl acetate. The combined organic layer was treated as in example Intermediate-1 step c using 0-10% ethyl acetate in hexane as eluent to obtain the title product (0.90 g, 83.86%). ¹H-NMR: NMR (400 MHz, DMSO-d6): δ 7.59 (s, 1H), 7.57 (d, 1H), 7.27 (dd, 1H), 5.56 (d, 1H), 4.93-4.88 (m, 1H), 1.3 (d, 3H).

### b) 1-(2-Bromo-5-chlorophenyl) ethan-1-one (146c)

To a solution of compound 146b (0.80 g, 3.40 mmol) in DCM (15.0 mL) was added DMP (2.16 g, 5.09 mmol) at 0° C. The mixture was slowly warmed to RT and stirred for 2 h. The mixture was diluted with hexane, filtered through celite pad, and washed with diethyl ether, dried over anhydrous sodium sulphate, filtered, and concentrated to afford the title compound (0.700 g, 88.28% g). ¹H-NMR: NMR (400 MHz, DMSO-d6): δ 7.77 (d, 1H), 7.73 (d, 1H), 7.50 (dd, 1H), 2.50 (s, 3H).

### c) (E)-1-(2-Bromo-5-chlorophenyl)-3-(dimethyl amino) prop-2-en-1-one (146d)

A mixture of compound 146c (8.60 g, 36.832 mmol) and dimethoxymethyl-dimethylazanium (6.72 g, 55.91 mmol) in sealed tube was heated at 120° C. for 12 h. The mixture was concentrated and diluted with water, extracted with ethyl acetate. The combined organic layer was treated as in example Intermediate-1 step c using 10-30% ethyl acetate in hexane as eluent to obtain the title compound (9.0 g, 84.68%). LCMS: 288.0 [M+H]⁺.

### d) 3-(2-Bromo-5-chlorophenyl)-1H-pyrazole (146e)

To a stirred solution of compound 146d (9.00 g, 31.19 mmol) in EtOH (90.0 mL), was added hydrazine hydrate (2.34 g, 46.78 mmol) at RT followed by stirring for 12 h. The mixture was concentrated and diluted with water and extracted with ethyl acetate. The combined organic layer was treated as in example Intermediate-1 step c using 10-15% ethyl acetate in hexane as eluent to obtain the title compound (8.0 g, 99.61%). LCMS: 257.0 [M+H]⁺.

### e) 6-(4-Chloro-2-(1H-pyrazol-3-yl)phenyl)isoindolin-1-one (Intermediate-146)

The compound was prepared according to the procedure described for Intermediate-139 with appropriate variations in reactants, quantities of reagents, and solvents. LCMS: 310.1 [M+H]⁺.

### Intermediate-147. 6-(4-Fluoro-2-(oxazol-2-yl)phenyl)isoindolin-1-one

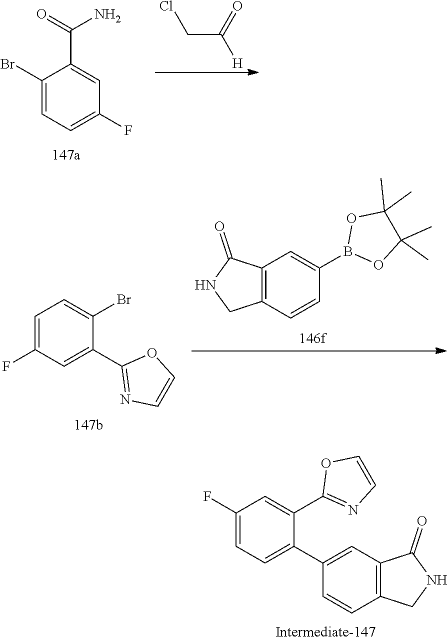

### a) 2-(2-Bromo-5-fluorophenyl)oxazole (147b)

A mixture of 2-bromo-5-fluorobenzamide (7.0 g, 32.00 mmol) and 2-chloro-acetaldehyde (3.02 g, 38.53 mmol) in acetic acid (15.0 mL) was heated at 120° C. for 8 h. The mixture was diluted with aqueous sodium bicarbonate solution and extracted with ethyl acetate. The combined organic layer was treated as in example Intermediate-1 step c using 50% ethyl acetate in hexane as eluent to obtain the title compound (0.7 g, 9.01%). LCMS: 244.0 [M+2+H]⁺.

### b) 6-(4-Fluoro-2-(oxazol-2-yl)phenyl)isoindolin-1-one (Intermediate-147)

The compound was prepared according to the procedure described for Intermediate-146 with appropriate variations in reactants, quantities of reagents, and solvents. LCMS: 295.0 [M+H]⁺.

### Intermediate-148. 6-(4-Fluoro-2-(4-(methyl-d3)-4H-1,2,4-triazol-3-yl)phenyl)-isoindolin-1-one

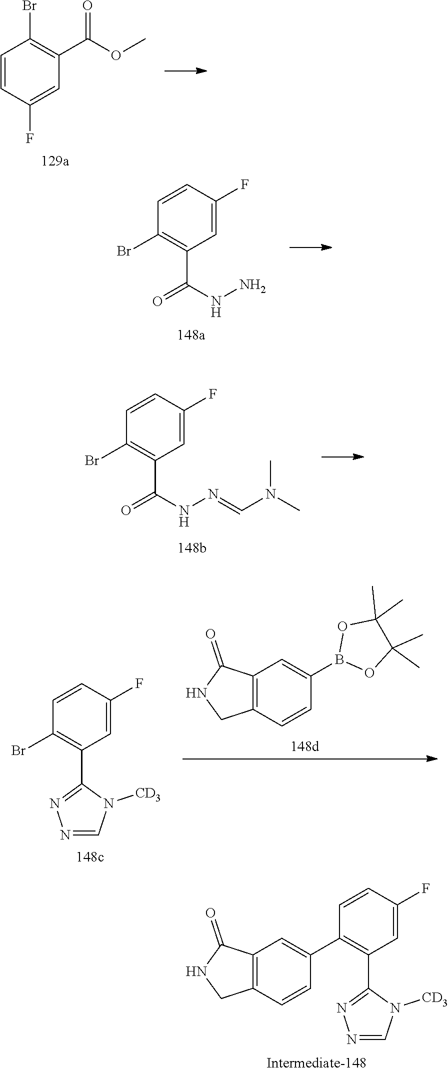 [SMILES: COC(=O)c1cc(F)ccc1Br ; NNC(=O)c1cc(F)ccc1Br ; CN(C)/C=N/NC(=O)c1cc(F)ccc1Br ; [2H]C([2H])([2H])n1cnnc1-c1cc(F)ccc1Br ; CC1(C)OB(c2ccc3c(c2)C(=O)NC3)OC1(C)C ; [2H]C([2H])([2H])n1cnnc1-c1cc(F)ccc1-c1ccc2c(c1)C(=O)NC2]

### a) 2-Bromo-5-fluorobenzohydrazide (148a)

The compound was prepared according to the procedure described for Intermediate-93b with appropriate variations in reactants, quantities of reagents, and solvents. LCMS: 232.9 [M+H]⁺.

### b) (E)-N′-(2-Bromo-5-fluorobenzoyl)-N,N-dimethylformohydrazonamide (148b)

To a solution of compound 148a (5.0 g, 21.45 mmol) in DCM (50.0 mL) was added DMF-DMA (10.2 g, 85.82 mmol) followed by stirring at 40° C. for 4 h. The mixture was cooled to RT, quenched with water, and extracted with ethyl acetate. The combined organic layer was dried over anhydrous sodium sulphate, filtered, and concentrated to afford the crude compound. The crude product was washed with diethyl ether to afford the title compound (6.1 g, 98.68%). LCMS: 290.0 [M+2+H]⁺.

### c) 3-(2-Bromo-5-fluorophenyl)-4-(methyl-d3)-4H-1,2,4-triazole (148c)

To a solution of compound 148b (5.0 g, 17.35 mmol) and CD₃NH₂·HCl (9.79 g, 138.83 mmol) in THF (100.0 mL) was added AcOH (20 ml) and stirred at 80° C. for 12 h. The mixture was cooled to RT, quenched with water, and extracted with 20% MeOH in DCM. The combined organic layer was treated as in example Intermediate-124 step d using 5% MeOH in DCM as eluent to afford the title compound (1.9 g, 42.26%). LCMS: 261.0 [M+2+H]⁺.

### d) 6-(4-Fluoro-2-(4-(methyl-d3)-4H-1,2,4-triazol-3-yl)phenyl)isoindolin-1-one (Intermediate-148)

The compound was prepared according to the procedure described for Inter-mediate-93 with appropriate variations in reactants, quantities of reagents, and solvents. LCMS: 312.1 [M+H]⁺.

### Intermediate-149. 6-(4-Fluoro-2-(4-methylisoxazol-3-yl)phenyl)isoindolin-1-one

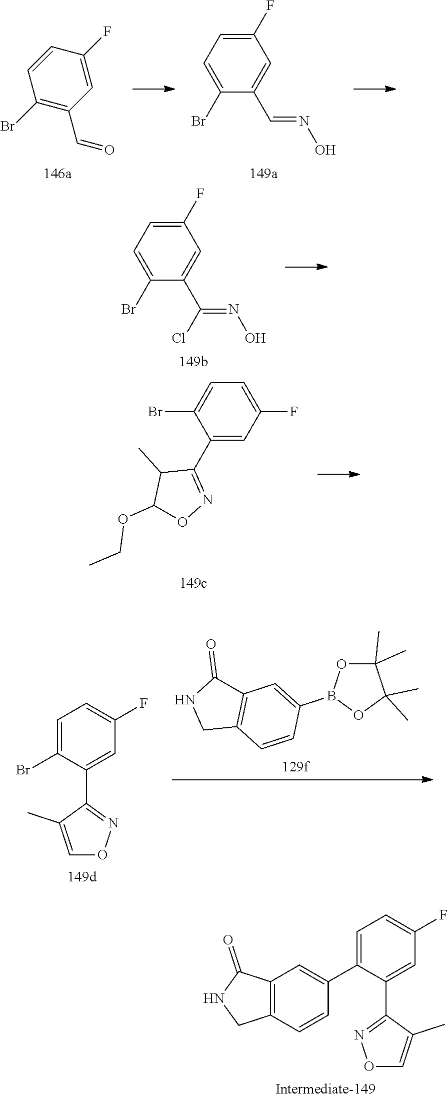

### a) (E)-2-Bromo-5-fluorobenzaldehyde oxime (149a)

To a solution of 2-bromo-5-fluorobenzaldehyde (2.0 g, 9.85 mmol) and NH₂OH·HCl (1.03 g, 14.77 mmol) in EtOH (20 mL) was added NaHCO₃ (0.99 g, 11.82 mmol) in water (10 mL) at 0° C. The mixture was stirred for 12 hr at RT. The mixture was concentrated, diluted with brine, and extracted with DCM. The combined organic layer was treated as in example Intermediate-128 step a to afford the title compound as off-white solid (2.0 g, 93.1%). LCMS: 218.0 [M+H]⁺.

### b) (Z)-2-Bromo-5-fluoro-N-hydroxybenzimidoyl chloride (149b)

To a solution of compound 149a (1.6 g, 7.34 mmol) in DMF (20 mL) was added NCS (0.98 g, 7.33 mmol) at 0° C. The mixture was stirred at 40° C. for 2 h. The mixture was diluted with ice cold water and extracted with ethyl acetate. The combined organic layer was treated as in example Intermediate-128 step a to afford the title compound as yellow liquid (1.6 g, 86.36%). The crude product was proceeded for next step as such.

### c) 3-(2-Bromo-5-fluorophenyl)-5-ethoxy-4-methyl-4,5-dihydroisoxazole (149c)

To a solution of compound 149b (1.6 g, 6.33 mmol) and (E)-1-methoxyprop-1-ene (1.09 g, 12.67 mmol) in DMF (20 mL) was added sodium bicarbonate (1.06 g, 12.67 mmol) at 0° C. The mixture was stirred at RT for 12 h. The reaction mixture was quenched with water and extracted with ethyl acetate. The combined organic layer was treated as in example Int-128 step a to afford the title compound as off white solid (1.5 g, 78.36%). LCMS: 302.0 [M+H]⁺.

### d) 3-(2-Bromo-5-fluorophenyl)-4-methylisoxazole (149d)

A mixture of compound 149c (1.4 g, 4.63 mmol) and TFA (2.0 mL) was heated at 80° C. for 10 h. The reaction mixture was quenched with water and extracted with ethyl acetate. The combined organic layer was treated as in example Intermediate-1 step c using 10-15% ethyl acetate in hexane as eluent to afford the title compound as off white solid (1 g, 84.27%). LCMS: 258.0 [M+2+H]⁺.

### e) 6-(4-Fluoro-2-(4-methylisoxazol-3-yl)phenyl)isoindolin-1-one (Intermediate-149)

The compound was prepared according to the procedure described for Intermediate-93 with appropriate variations in reactants, quantities of reagents, and solvents. LCMS: 309.1 [M+H]⁺.

### Intermediate-150. 6-(4-Fluoro-2-(1-methyl-1H-imidazol-2-yl)phenyl)iso-indolin-1-one

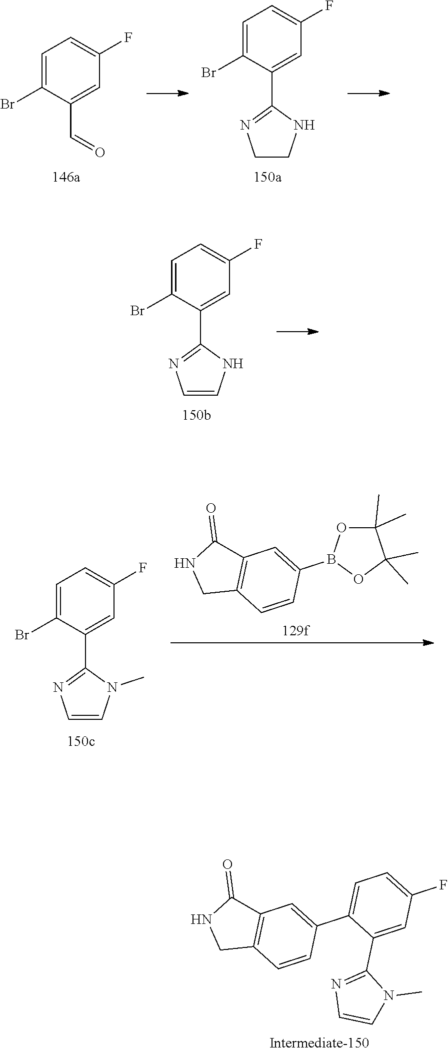 [SMILES: O=Cc1cc(F)ccc1Br ; Fc1ccc(Br)c(C2=NCCN2)c1 ; Fc1ccc(Br)c(-c2ncc[nH]2)c1 ; Cn1ccnc1-c1cc(F)ccc1Br ; CC1(C)OB(c2ccc3c(c2)C(=O)NC3)OC1(C)C ; Cn1ccnc1-c1cc(F)ccc1-c1ccc2c(c1)C(=O)NC2]

### a) 2-(2-Bromo-5-fluorophenyl)-4,5-dihydro-1H-imidazole (150a)

To a solution of 2-bromo-5-fluorobenzaldehyde (3.0 g, 14.77 mmol) in t-BuOH (10 mL) was added ethylenediamine (0.98 g, 16.25 mmol) followed by stirring for 30 min in RT. Then K₂CO₃ (0.99 g, 11.82 mmol) and 12 (0.99 g, 11.82 mmol) was added to followed by stirring for 12 h at 70° C. The reaction mixture was quenched with saturated aqueous solution of Na₂SO₃ until the iodine colour disappeared and then extracted with ethyl acetate. The combined organic layer was treated as in example Intermediate-128 step a to afford the title compound as yellow solid (3.50 g, 97.4%) LCMS: 245.0 [M+2+H]⁺.

### b) 2-(2-Bromo-5-fluorophenyl)-1H-imidazole (150b)

To a solution of compound 150a (2.8 g, 11.58 mmol) in DMSO (20.0 mL) was added K₂CO₃ (1.07 g, 12.67 mmol) and iodobenzene diacetate (4.08 g, 12.67 mmol) and stirred at RT for 12 h. The mixture was diluted with water and extracted with ethyl acetate. The combined organic layer was treated as in example Intermediate-1 step a using 50% ethyl acetate in hexane as eluent to afford the title compound as off white solid (1.0 g, 36%). LCMS: 241.0 [M+H]⁺.

### c) 2-(2-Bromo-5-fluorophenyl)-1-methyl-1H-imidazole (150c)

To a solution of compound 150b (1.0 g, 4.16 mmol) in THF (10 mL) was added NaH (0.11 g, 4.56 mmol), followed by iodomethane (0.65 g, 4.56 mmol) at 0° C. The mixture was stirred for 12 h at RT. The mixture was diluted with water and extracted with ethyl acetate. The combined organic layer was treated as in example Intermediate-128 step a to afford the title compound as transparent liquid. (0.80 g, 75.6%), LCMS: 255.0 [M+H]⁺.

### d) 6-(4-Fluoro-2-(1-methyl-1H-imidazol-2-yl)phenyl)isoindolin-1-one (Inter-mediate-150)

The compound was prepared according to the procedure described for Intermediate-93 with appropriate variations in reactants, quantities of reagents, and solvents. LCMS: 308.1 [M+H]⁺.

### Intermediate-151. 1-(2-Bromo-6-methylpyridin-4-yl)-N-(cyclopropylmethyl)-methanamine (Procedure A′)

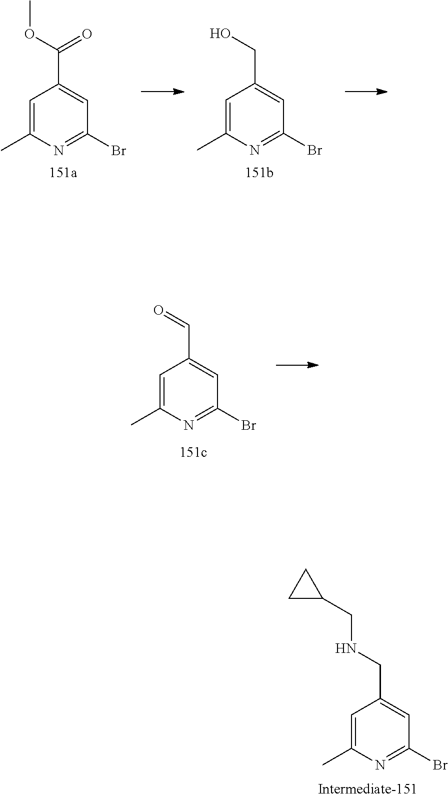

### a) (2-Bromo-6-methylpyridin-4-yl)methanol (151b)

To a solution of methyl 2-bromo-6-methylisonicotinate (25 g, 108.67 mmol) in THF (250 mL) was added LiBH₄ (217.34 mL, 434.68 mmol, 2 M in THF) dropwise at −78° C. The mixture was slowly warmed to RT and stirred for 4 h. The mixture was cooled to 0° C., quenched with saturated aqueous ammonium chloride followed by water (250.0 mL), and extracted with ethyl acetate. Then combined organic layer was treated as in example Intermediate-128 step a to obtain the title compound (22.0 g). ¹H-NMR (400 MHz, DMSO-d6): 7.34 (d, 1H), 7.22 (d, 1H), 5.51 (t, 1H), 4.49 (d, 2H), 2.43 (s, 3H).

### b) 2-Bromo-6-methylisonicotinaldehyde (151c)

To a solution of compound 151b (22.0 g, 108 mmol) in DCM (200 mL) was added Dess-Martin periodinane (69 g, 163.32 mmol) portion wise at 0° C. The mixture was slowly warmed to RT and stirred for 6 h. The mixture was cooled to 0° C., quenched with aqueous sodium thiosulphate solution and aqueous sodium bicarbonate solution, and extracted with DCM. The combined organic layer was treated as in example Intermediate-1 step c using 10-30% ethyl acetate in hexane as eluent to obtain the title compound (18.0 g). ¹H-NMR (400 MHz, DMSO-d6): 10.0 (s, 1H), 7.86 (d, 1H), 7.71 (d, 1H), 2.57 (s, 3H).

### c) 1-(2-Bromo-6-methylpyridin-4-yl)-N-(cyclopropylmethyl)methanamine (Intermediate-151)

To a solution of cyclopropylmethanamine (3.73 g, 52.49 mmol) and compound 151c (7.0 g, 34.99 mmol) in DCM (100 mL) was added a catalytic amount of acetic acid and stirred for 1 h. STAB (14.83 g, 69.98 mmol) was added at 0° C. and followed by stirring at RT for 12 h. The reaction mixture was quenched with water and extracted with DCM. The combined organic layer was was treated as in example Intermediate-1 step c using 60% ethyl acetate in hexane as eluent to afford the title compound (4.1 g, 46%). LCMS: 255.1 [M+H]⁺.

### Intermediate-152. 1-(2-Bromo-6-methylpyridin-4-yl)-N-(cyclobutylmethyl)-methanamine (Procedure B′)

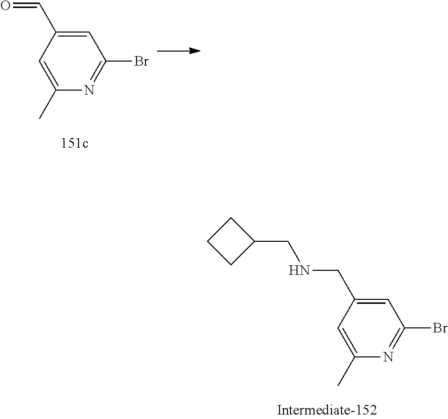

To a solution of cyclobutylmethanamine hydrochloride (0.28 g, 2.25 mmol) in DCM (10 mL) was added DIPEA (0.58 mL, 4.50 mmol) and stirred for 30 min. To this mixture was added 2-bromo-6-methylisonicotinaldehyde (0.30 g, 1.50 mmol) followed by STAB (0.64 g, 3.00 mmol) at 0° C. and stirred at RT for 12 h. The mixture was quenched with water and extracted with DCM. The combined organic layer was treated as in example Intermediate-1 step c using 0-30% ethyl acetate in hexane as eluent to afford the title compound (0.33 g, 82%). LCMS: 271.1 [M+2+H]⁺.

### Intermediate-155. 1-(2,6-Dichloropyridin-4-yl)-N-((1-methylcyclobutyl)-methyl)methanamine (Procedure C′)

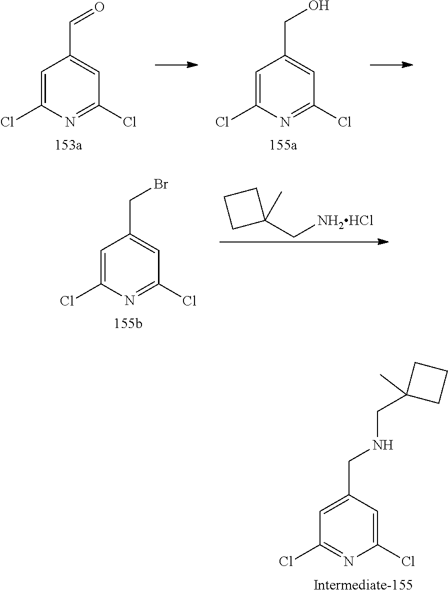

### a) (2,6-Dichloropyridin-4-yl)methanol (155a)

To a solution of 2,6-dichloroisonicotinaldehyde (3.0 g, 17.04 mmol) in MeOH (20 mL) was added NaBH4 (1.26 g, 34.09 mmol) at 0° C. and stirred for 1 h. The reaction mixture was quenched by saturated aqueous ammonium chloride solution and extracted by ethyl acetate. The combined organic layer was treated as in example Intermediate-1 step c using 0-5% ethyl acetate in hexane as eluent to afford the title compound (2.7 g, 90%). LCMS: 178.0 [M+H]⁺.

### b) 4-(Bromomethyl)-2,6-dichloropyridine (155b)

To a solution of compound 155a (2.7 g, 15.16 mmol) in DCM (30 mL) was added triphenylphosphine (4.77 g, 18.2 mmol) and carbon tetrabromide (6.03 g, 18.2 mmol) at 0° C. and stirred at RT for 3 h. The mixture was diluted with water and extracted with DCM. The combined organic layer was treated as in example Intermediate-1 step c using 0-5% ethyl acetate in hexane as eluent to afford the title compound. LCMS: 242.0 [M+2+H]⁺.

### c) 1-(2,6-Dichloropyridin-4-yl)-N-((1-methylcyclobutyl)methyl)methanamine-amine (Intermediate-155)

To a solution of compound 155b (0.6 g, 2.49 mmol) and (1-methylcyclobutyl) methenamine (0.45 g, 2.98 mmol) in MeCN (3 mL) was added, K₂CO₃ (0.86 g, 6.22 mmol) followed by stirring at RT for 12 h. The mixture was diluted with water and extracted with ethyl acetate. The combined organic layer was treated as in example Intermediate-128 step a to afford the title compound (0.6 g, 92%) LCMS: 259.9 [M+H]⁺.

### Intermediate-156. 4-((2-Chloro-6-(trifluoromethyl)pyridin-4-yl)methyl)-2-methyl-4,5,6,7-tetrahydro-2H-pyrazolo[4,3-b]pyridine

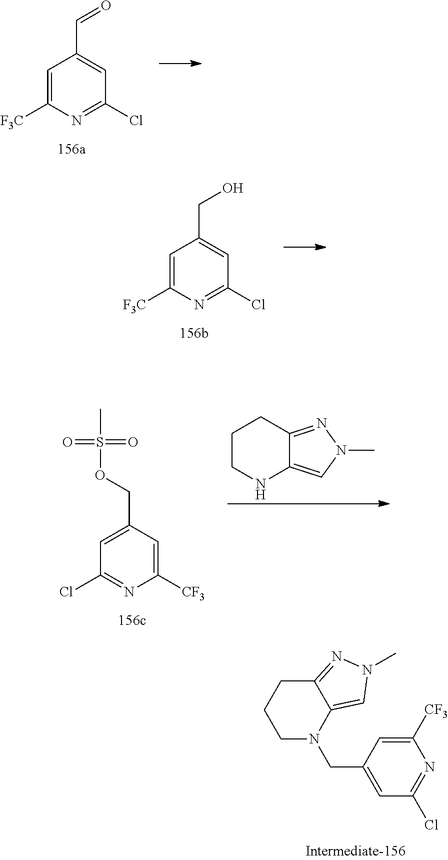

### a) (2-Chloro-6-(trifluoromethyl) pyridin-4-yl) methanol (156b)

To a solution of 2-chloro-6-(trifluoromethyl)isonicotinaldehyde (1.0 g, 4.77 mmol) in THF (10 mL) was added sodium borohydride (0.36 g, 9.54 mmol) portion wise at 0° C. The mixture was slowly warmed to RT followed by stirring for 2 h. The reaction mixture was quenched with aqueous ammonium chloride solution and extracted with ethyl acetate. The combined organic layer was treated as in example Intermediate-1 step c using 20-30% ethyl acetate in hexane as eluent to afford the title compound (0.97 g, 96.08%). LCMS: 212.0 [M+H]⁺.

### b) (2-Chloro-6-(trifluoromethyl) pyridin-4-yl)methyl methane sulfonate (156c)

To a solution of compound 156b (0.35 g, 1.68 mmol) and triethylamine (0.51 g, 5.06 mmol) in DCM (6 mL) was added methanesulfonyl chloride (0.26 mL, 3.37 mmol) at 0° C. followed by stirring at RT for 1 h. The reaction mixture was quenched with water and extracted with DCM. The combined organic layer was treated as in example Intermediate-128 step a to afford the title compound (0.6 g) as crude product which was used in next step as such. LCMS: 289.9 [M+H]⁺.

### c) 4-((2-Chloro-6-(trifluoromethyl)pyridin-4-yl)methyl)-2-methyl-4,5,6,7-tetra-hydro-2H-pyrazolo[4,3-b]pyridine (Intermediate-156)

To the stirred solution of 2-methyl-4,5,6,7-tetrahydro-2H-pyrazolo[4,3-b]pyridi-ne (0.1 g, 0.73 mmol) (prepared according to WO 2014/029732) in THF (5 ml) was added cesium carbonate (0.5 g, 1.82 mmol) at 0° C. followed by stirring for 30 min. Compound 156c (0.27 g, 0.87 mmol) was added to the reaction mixture at 0° C. and then stirred for 12 h at room temperature. The reaction mixture quenched with brine, and extracted with ethyl acetate. The combined organic layer was treated as in example Intermediate-1 step c using 50% ethyl acetate in hexane as eluent to afford the title compound (0.050 g, 20%). LCMS: 332.1 [M+2+H]⁺.

### Intermediate-157. 2,6-Dichloro-4-((4,4-difluoro-3-methylpiperidin-1-yl)-methyl)pyridine

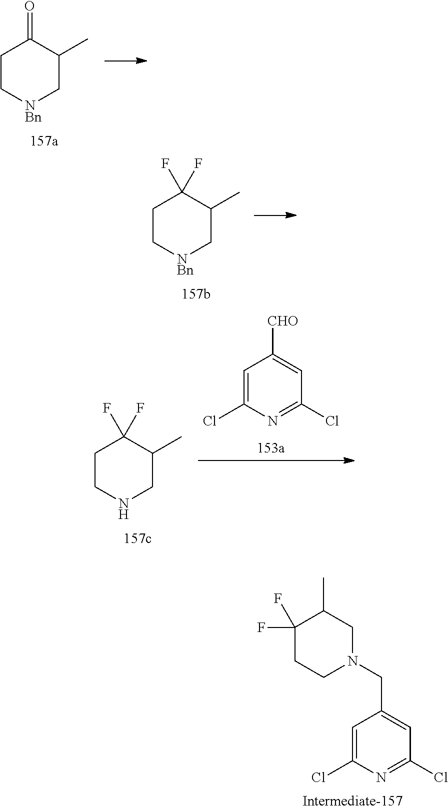

### a) 1-Benzyl-4,4-difluoro-3-methylpiperidine (157b)

To a solution of 1-benzyl-3-methylpiperidin-4-one (1.0 g, 4.918 mmol) in DCM (10 mL) was added (diethylamino) sulphur trifluoride (3.96 g, 24.59 mmol) followed by stirring at RT for 16 h. The reaction mixture was quenched by saturated sodium bi-carbonate and extracted with ethyl acetate. The combined organic layer was dried over anhydrous sodium sulphate, filtered, and concentrated to afford the crude material. The crude was purified by silica-gel flash column chromatography using 10-20% ethyl acetate in hexane as eluent to obtain the title product (0.9 g, 81%). LCMS: 226.1 [M+H]⁺.

### b) 4,4-Difluoro-3-methylpiperidine (157c)

To a solution of compound 157b (0.9 g, 3.995 mmol) in ethanol (10 ml) was added Pd/C (0.43 g, 0.400 mmol) followed by stirring at RT under hydrogen atmosphere for 24 h. The mixture was filtered through celite pad and washed with DCM. The filtrate was concentrated to afford the title product (0.750 g, 75%). LCMS: 136.1 [M+H]⁺.

### c) 2,6-Dichloro-4-((4,4-difluoro-3-methylpiperidin-1-yl)methyl)pyridine (Intermediate-157)

The compound was prepared according to the procedure described for Intermediate 152 with appropriate variations in reactants, quantities of reagents, and solvents. LCMS: 295.0 [M+H]⁺.

### Intermediate-158. 2,6-Dichloro-4-((3-fluoro-3-methylpyrrolidin-1-yl)-methyl)pyridine

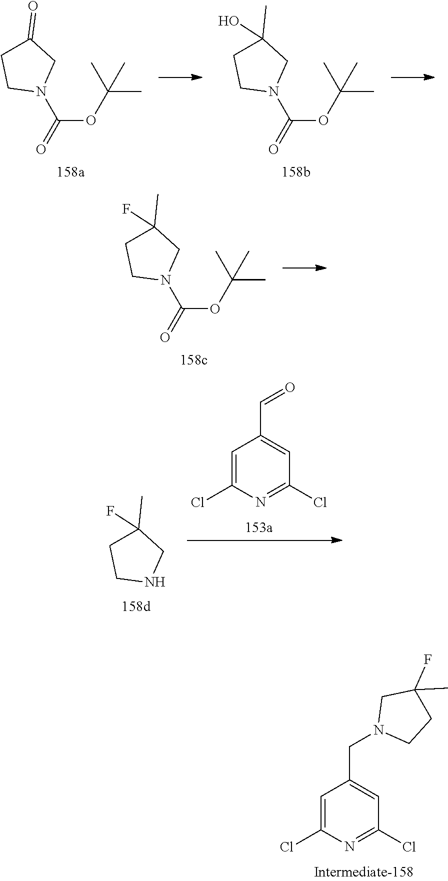

### a) tert-Butyl 3-hydroxy-3-methylpyrrolidine-1-carboxylate (158b)

To a solution of tert-butyl 3-oxopyrrolidine-1-carboxylate (2 g, 10.81 mmol) in diethyl ether (20 mL) was added methyl magnesium bromide (7.2 mL, 21.59 mmol, 3 M in diethyl ether) at 0° C. dropwise followed by stirring at 0° C. for 2 h. The reaction mixture was quenched by saturated aqueous ammonium chloride solution and extracted by ethyl acetate. The combined organic layer was treated as in example Intermediate-1 step c using 0-5% ethyl acetate in hexane as eluent to afford the title compound (1.4 g, 64%). LCMS: 102.0 [M−100+H]⁺.

### b) tert-Butyl 3-fluoro-4-methylpyrrolidine-1-carboxylate (158c)

To a solution of compound 158b (1.2 g, 5.96 mmol) in dried DCM was added diethylaminosulfur trifluoride (1.92 g, 11.92 mmol) under argon atmosphere at 0° C. The mixture was slowly warmed to RT and stirred for 1 h. The reaction mixture was quenched with saturated aqueous NaHCO₃ solution and extracted with DCM. The combined organic layer was treated as in example Intermediate-1 step c using 15-17% ethyl acetate in hexane as eluent to obtain the desired product (0.8 g, 66%).

### c) 3-Fluoro-4-methylpyrrolidine (158d)

To a solution of compound 158c (0.1 g, 0.49 mmol) in DCM (0.3 mL) was added TFA (0.5 mL) at 5° C. followed by stirring at RT for 16 h. The reaction mixture was quenched with saturated aqueous NaHCO₃ solution and extracted with DCM. The combined organic layer was treated as in example Intermediate-128 step a to afford the title compound (0.08 g) as crude product which was used in next step as such. LCMS: 104.2 [M+H]⁺.

### d) 2,6-Dichloro-4-(3-fluoro-4-methylpyrrolidin-1-yl)pyridine (Intermediate-158)

The compound was prepared according to the procedure described for Intermediate-152 with appropriate variations in reactants, quantities of reagents, and solvents. LCMS: 263.0 [M+H]⁺.

### Intermediate-159. 2,6-Dichloro-4-((3-fluoro-4-methylpyrrolidin-1-yl)methyl)-pyridine

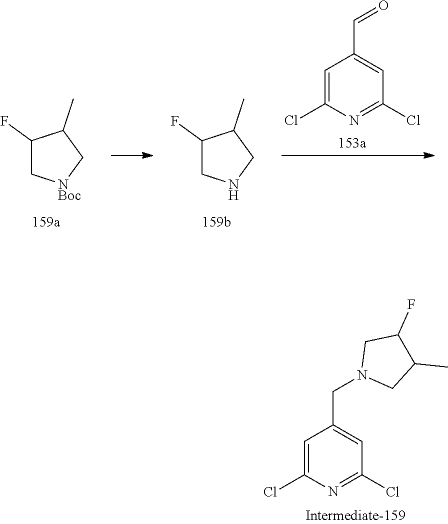

### a) 3-Fluoro-4-methylpyrrolidine (159b)

To a solution of tert-butyl 3-fluoro-4-methylpyrrolidine-1-carboxylate (1.60 g, 7.87 mmol) (prepared according to WO 2014/177060) in dioxane (5.0 mL) was added 4 M HCl in dioxane (15.0 mL) at 0° C. followed by stirring at RT for 16 h. The reaction mixture was quenched with saturated aqueous NaHCO₃ solution and extracted with DCM. The combined organic layer was treated as in example Intermediate-128 step a to afford the title compound (1.10 g) as crude product which was used in next step as such. LCMS: 104.2 [M+H]⁺.

### b) 2,6-Dichloro-4-((3-fluoro-4-methylpyrrolidin-1-yl)methyl)pyridine (Inter-mediate-159)

The compound was prepared according to the procedure described for Intermediate-152 with appropriate variations in reactants, quantities of reagents, and solvents. LCMS: 263.0 [M+H]⁺.

### Intermediate-160. 1-(6-Chloro-3-fluoro-2-methylpyridin-4-yl)-N-(cyclopropylmethyl)methanamine

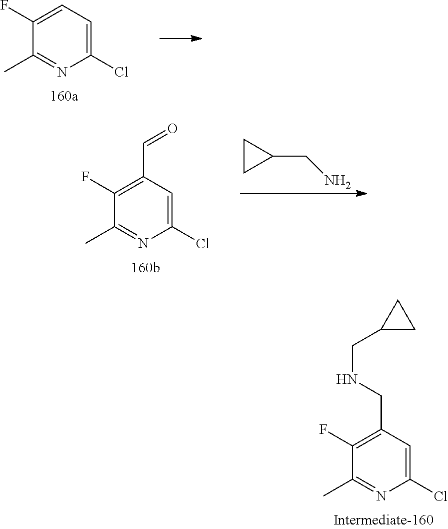

### a) 2,6-Dichloro-3-fluoroisonicotinaldehyde (45b)

To a solution of 6-chloro-3-fluoro-2-methylpyridine (5.0 g, 34.35 mmol) in dry THF (100 mL) was added 2,2,6,6-tetramethyl piperidinyl magnesium chloride lithium chloride complex (16.65 g, 68.70 mmol) dropwise at 0° C. followed by stirring at RT for 1 h. The mixture was cooled to −78° C. and dry DMF (5.0 mL in 25 mL THF) was added. The mixture was slowly warmed to RT and stirred for 16 h. The mixture was cooled to 0° C., quenched with aqueous ammonium chloride solution and extracted with ethyl acetate. The combined ethyl acetate layer was treated as in example Intermediate-1 step c using 0-10% ethyl acetate in hexane as eluent to afford the title compound as a liquid (2.70 g, 45.29%). LCMS: 172.2 [M−H]⁻.

### b) 1-(6-Chloro-3-fluoro-2-methylpyridin-4-yl)-N-(cyclopropylmethyl)methan-amine (Intermediate-160)

The compound was prepared according to the procedure described for Intermediate-151 with appropriate variations in reactants, quantities of reagents, and solvents. LCMS: 229.1 [M+H]⁺.

### Intermediate-161. 2,6-Dichloro-4-(4-methylpiperidin-2-yl)pyridine

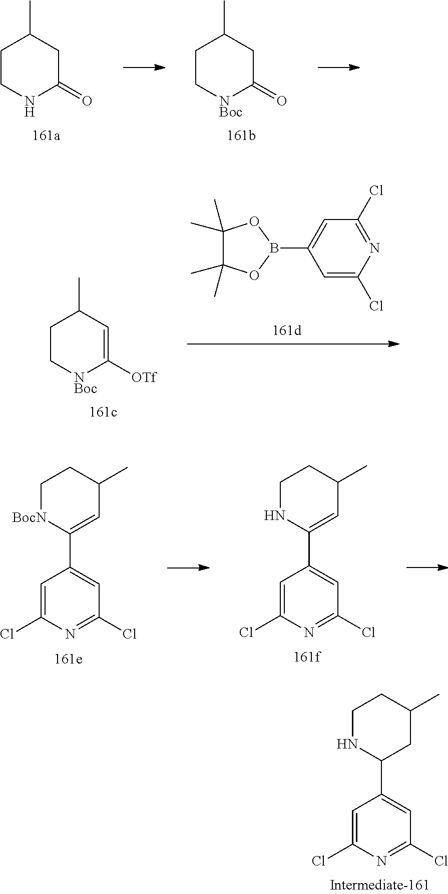

### a) tert-Butyl 4-methyl-2-oxopiperidine-1-carboxylate (161b)

To a solution of 4-methylpiperidin-2-one (5.00 g, 44.19 mmol) and 4-dimethyl-aminopyridine (5.43 g, 44.18 mmol) in DCM (50.0 mL) was added DIPEA (6.16 mL, 88.37 mmol) and di-tert-butyl di-carbonate (19.5 mL, 88.37 mmol) dropwise at 0° C. followed by stirring at RT for 12 h. The reaction mixture was quenched with saturated aqueous ammonium chloride and extracted with ethyl acetate. The combined organic layer was treated as in example Intermediate-1 step c to afford the title compound (8.4 g, 89.14%). LCMS: 214.1 [M+H]⁺.

### b) tert-Butyl 4-methyl-6-(((trifluoromethyl)sulfonyl)oxy)-3,4-dihydropyridine-1(2H)-carboxylate (161c)

To a stirred solution of compound 161b (8.4 g, 39.38 mmol) in THF (80.0 mL) was added LDA (5.06 g, 47.26 mmol) dropwise at −78° C. over 15 min followed by a solution of N-phenyl-bis(trifluoromethanesulfonimide) (16.88 g, 47.26 mmol) in THF (20 mL) dropwise. The mixture was slowly warmed to RT and stirred for 7 h. The reaction mixture was quenched with saturated aqueous ammonium chloride and extracted with ethyl acetate. The combined organic layer was treated as in example Intermediate-1 step c using 10-20% ethyl acetate in hexane as eluent to afford the title compound (2.5 g, 18.38%). LCMS: 346.1 [M+H]⁺.

### c) tert-Butyl 2′,6′-dichloro-4-methyl-5,6-dihydro-[2,4′-bipyridine]-1(4H)-carboxylate (161e)

To a degassed solution of compound 161c (2.3 g, 6.66 mmol), 2,6-dichloro-4-(4,4,5,5-tetramethyl-1,3,2-dioxaborolan-2-yl)pyridine (2.18 g, 7.99 mmol) and K₂CO₃ (1.41 g, 13.32 mmol) in dioxane (30.0 mL) and water (10.0 mL) was added Pd(dppf)Cl₂ (0.244 g, 0.33 mmol) followed by stirring at 100° C. for 4 h. The mixture was cooled to RT, quenched with water, and extracted with ethyl acetate. The combined organic layer was treated as in example Intermediate-1 step c using 10% ethyl acetate in hexane as eluent to afford the title compound (2.0 g, 87.49%). LCMS: 345.1 [M+H]⁺.

### d) 2′,6′-Dichloro-4-methyl-1,4,5,6-tetrahydro-2,4′-bipyridine (161f)

To a solution of compound 161e (0.81 g, 2.37 mmol) in DCM (10.0 mL) was added TFA (1.08 g, 9.49 mmol) and stirred at RT for 2 h. The mixture was concentrated to dryness and the obtained solid was washed with diethyl ether to afford the title compound (0.55 g, 95.29%) LCMS: 243.1 [M+H]⁺.

### e) 2,6-Dichloro-4-(4-methylpiperidin-2-yl)pyridine (Intermediate-161)

To a solution of compound 161f (0.550 g, 2.26 mmol) in THF (10.0 mL) was added TEA (1.33 g, 13.16 mmol) and sodium cyano borohydride (0.413 g, 6.58 mmol) followed by stirring at RT for 12 h. The reaction mixture was quenched with water and extracted with ethyl acetate. The combined organic layer was treated as in example Intermediate-1 step c using 30% ethyl acetate in hexane as eluent to afford the title compound (0.250 g, 45.08%). LCMS: 245.0 [M+H]⁺.

### Intermediate-162. 1-(2,6-Dichloropyridin-4-yl)-N-((5-methyltetrahydrofuran-2-yl)methyl)methanamine

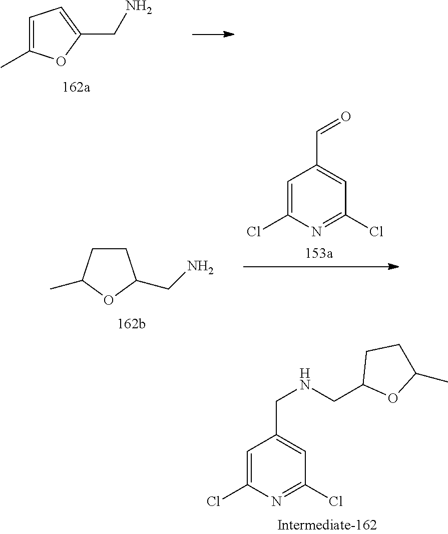

### a) (5-Methyltetrahydrofuran-2-yl)methanamine (162b)

To a solution of (5-methylfuran-2-yl)methanamine (0.80 g, 7.19 mmol) in ethyl acetate (30 mL) was added palladium on carbon (0.077 g, 0.720 mmol, 10% weight) and stirred under hydrogen (50 psi) at RT for 12 h. The mixture was filtered through celite pad. The filtrate was concentrated to afford the title compound (0.60 g, 72%).

### b) 1-(2,6-Dichloropyridin-4-yl)-N-((5-methyltetrahydrofuran-2-yl)methyl)-methanamine (Intermediate-162)

The compound was prepared according to the procedure described for Intermediate-151 with appropriate variations in reactants, quantities of reagents, and solvents. LCMS: 275.0 [M+H]⁺.

### Intermediate-163. 2-(2-Chloro-6-methylpyridin-4-yl)-N-(cyclopropylmethyl)-propan-2-amine

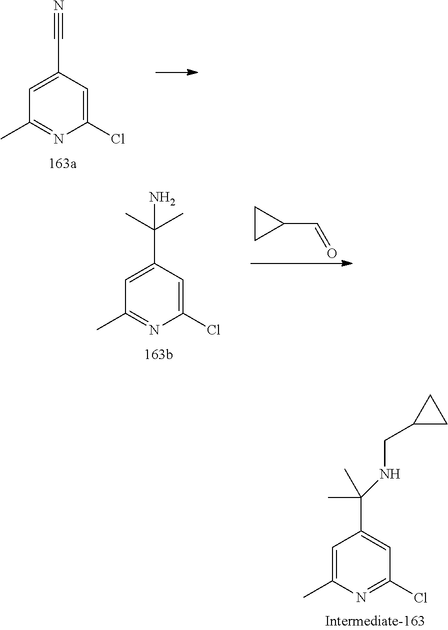

### a) 2-(2-Chloro-6-methylpyridin-4-yl)propan-2-amine (163b)

To a solution of 2-chloro-6-methylisonicotinonitrile (0.3 g, 1.62 mmol) in diethyl ether (10 mL) was added methyl magnesium bromide (0.73 g, 6.55 mmol) followed by Ti(OiPr)₄ (1.86, 6.55 mmol) dropwise at 0° C. followed by stirring for 1 h.

The mixture was heated at 60° C. for 12 h. The reaction mixture was quenched by saturated aqueous ammonium chloride solution and extracted with ethyl acetate. The combined organic layer was treated as in example Intermediate-1 step c using 0-5% ethyl acetate in hexane as eluent to afford the title compound (0.3 g, 49.57%) LCMS: 185.1 [M+H]⁺.

### b) 2-(2-Chloro-6-methylpyridin-4-yl)-N-(cyclopropylmethyl)propan-2-amine (Intermediate-163)

To a solution of compound 163b (0.3 g, 1.62 mmol) in MeOH (50 mL) was added cyclopropanecarbaldehyde (0.34 g, 4.87 mmol) and stirred at RT for 3 h. NaBH₄ (0.037 g, 0.970 mmol) was added at 0° C. followed by stirring at RT for 16 h. The reaction mixture was quenched with water and extracted with DCM. The combined organic layer was treated as in example Intermediate-1 step c using 15-20% ethyl acetate in hexane as eluent to afford the title compound (0.3 g, 77.32%). LCMS: 239.1 [M+H]⁺.

### Intermediate-164. N-(Cyclopropylmethyl)-2-(2,6-dichloropyridin-4-yl)propan-2-amine

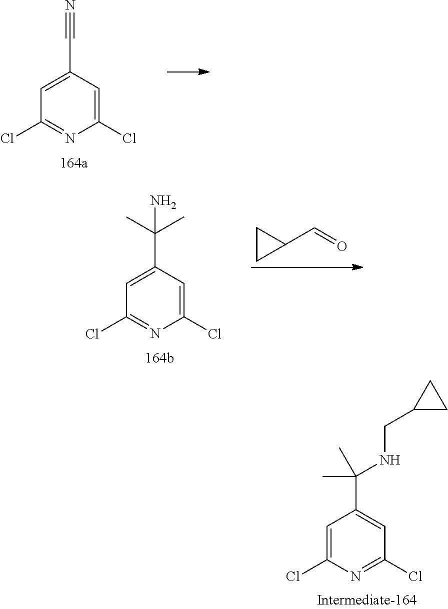 [SMILES: N#Cc1cc(Cl)nc(Cl)c1 ; CC(C)(N)c1cc(Cl)nc(Cl)c1 ; O=CC1CC1 ; CC(C)(NCC1CC1)c1cc(Cl)nc(Cl)c1]

### a) 2-(2,6-Dichloropyridin-4-yl)propan-2-amine

The compound was prepared according to the procedure described for Intermediate-163b with appropriate variations in reactants, quantities of reagents, and solvents. LCMS: 205.1 [M+H]⁺.

### b) N-(Cyclopropylmethyl)-2-(2,6-dichloropyridin-4-yl)propan-2-amine (Intermediate-164)

The compound was prepared according to the procedure described for Intermediate-163 with appropriate variations in reactants, quantities of reagents, and solvents. LC-MS: 259.1 [M+H]⁺.

### Intermediate-165. 1-(6-Chloro-2-methylpyrimidin-4-yl)-N-(cyclopropyl-methyl)methanamine

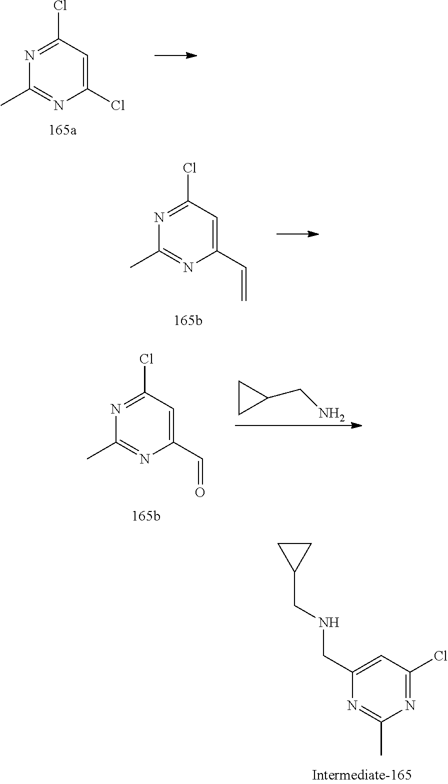

### a) 4-Chloro-2-methyl-6-vinylpyrimidine (165b)

To a degassed solution of 4,6-dichloro-2-methylpyrimidine (10.00 g, 61.35 mmol), potassium trifluoro(vinyl)borate (4.10 g, 30.67 mmol) and sodium carbonate (9.75 g, 92.02 mmol) in 1,4-dioxane (45.0 mL) and water (30.0 mL) was added bis(triphenylphosphine)palladium (II) dichloride (2.50 g, 3.06 mmol) followed by stirring at 70° C. for 2 h. The mixture was cooled to RT, filtered through celite pad, and washed with ethyl acetate. The filtrate was concentrated, diluted with water, and extracted with ethyl acetate. The combined organic layer was washed with brine, dried over anhydrous sodium sulphate, filtered, and concentrated, and purified by silica-gel flash column chromatography using 8-10% ethyl acetate in hexane as eluent to afford the title compound as a colourless liquid (1.50 g, 15%). LCMS: 155.1 [M+H]⁺.

### b) 6-Chloro-2-methylpyrimidine-4-carbaldehyde (165c)

To a solution of compound 165b (1.50 g, 9.70 mmol) and sodium periodate (3.87 g, 18.11 mmol) in THF (20.0 mL) and water (10.0 mL) was added osmium tetroxide (0.069 mL, 0.27 mmol, 4 wt-% in H₂O) dropwise at 0° C. The mixture was slowly warmed to RT and stirred for 4 h. The reaction mixture was quenched with ice water and extracted with ethyl acetate. The combined organic layer was washed with brine, dried over anhydrous sodium sulphate, filtered, concentrated, and purified by silica-gel flash column chromatography using 8-9% ethyl acetate in hexane as eluent to afford the title compound as a liquid (0.60 g, 39%). LCMS: 157.0 [M+H]⁺.

### c)—(6-Chloro-2-methylpyrimidin-4-yl)-N-(cyclopropylmethyl)methanamine (Intermediate-165)

The compound was prepared according to the procedure described for Inter-mediate-151 with appropriate variations in reactants, quantities of reagents, and solvents. LCMS: 212.1 [M+H]⁺.

### Intermediate-166. 4-((2,6-Dichloropyridin-4-yl)methyl)hexahydro-2H-furo[3,2-b]pyrrole

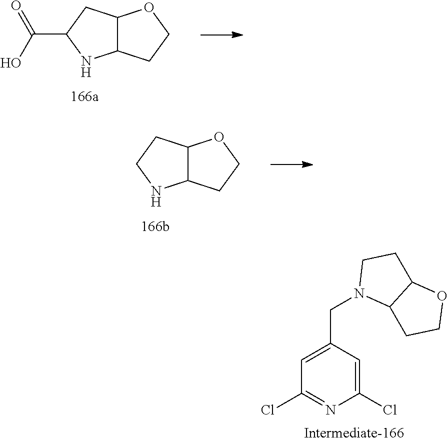 [SMILES: O=C(O)C1CC2OCCC2N1 ; C1CC2OCCC2N1 ; Clc1cc(CN2CCC3OCCC32)cc(Cl)n1]

### a) Hexahydro-2H-furo[3,2-b]pyrrole (166b)

To a solution of hexahydro-2H-furo[3,2-b]pyrrole-5-carboxylic acid (0.45 g, 2.86 mmol) (prepared according to WO 2022/217276) in cyclohexanol (4 mL) was added 4′-methylacetophenone (0.04 g, 0.28 mmol) followed by stirring at 160° C. for 4 h. The mixture was diluted with ethyl acetate and extracted with 1 N HCl. The combined aqueous layer was concentrated to get brown oil. The crude mixture was washed with diethyl ether and dried to afford the title compound as HCl salt in the form of brow oil (0.22 g, 50%). LCMS: 114.1 [M+H]⁺.

### b) 4-((2,6-Dichloropyridin-4-yl)methyl)hexahydro-2H-furo[3,2-b]pyrrole (Intermediate-166)

The compound was prepared according to the procedure described for Intermediate-152 with appropriate variations in reactants, quantities of reagents, and solvents. LCMS: 273.0 [M+H]⁺.

### Intermediate 167 and 168. N-((2-Bromo-6-methylpyridin-4-yl)methyl)-2-methylpropane-2-sulfinamide (Int-167) and N-((2-bromo-6-methylpyridin-4-yl)methyl)-methanesulfonamide (Int-168)

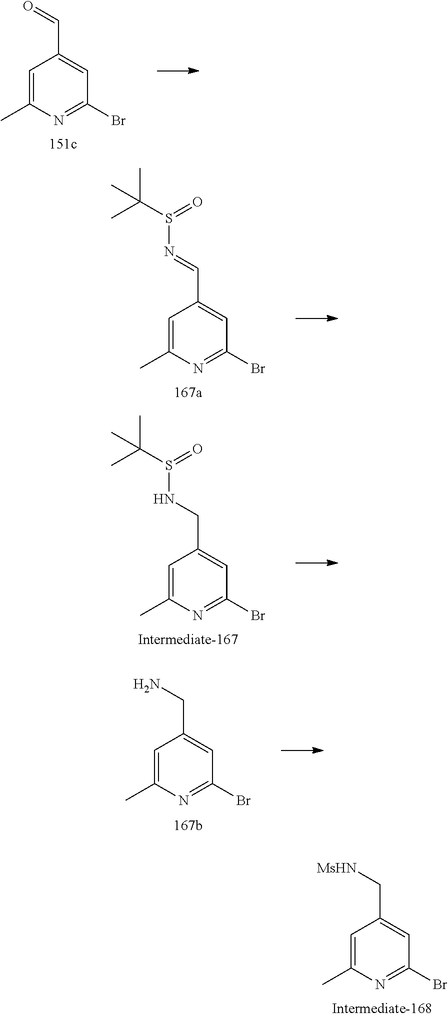

### a) (E)-N-((2-Bromo-6-methylpyridin-4-yl)methylene)-2-methylpropane-2-sulfinamide (167a)

To a solution of 2-bromo-6-methylisonicotinaldehyde (1.0 g, 4.99 mmol) and 2-methylpropane-2-sulfinamide (0.636 g, 5.25 mmol) in DCM (30.0 mL) was added titanium ethoxide (2.28 g, 9.99 mmol) at 0° C. followed by stirring at RT for 12 h. The reaction mixture was quenched with ice cold water and extracted with DCM. The combined organic layer was dried over anhydrous sodium sulphate, filtered, and concentrated to afford the title compound as off white solid (0.54 g, 35.7%); LCMS: 303.0 [M+H]⁺.

### b) N-((2-Bromo-6-methylpyridin-4-yl)methyl)-2-methylpropane-2-sulfinamide (Intermediate-167)

To a solution of compound 167a (0.4 g, 1.32 mmol) in MeOH (4.0 mL) was added sodium borohydride (0.1 g, 2.63 mmol) at 0° C. followed by stirring at RT for 4 h. The reaction mixture was quenched with 1 N HCl and extracted with ethyl acetate. The combined organic layer was treated as in example Intermediate-128 step a to afford the title compound as off-white solid (0.54 g, 35.7%); LCMS: 305.0 [M+H]⁺.

### c) (2-Bromo-6-methylpyridin-4-yl)methanamine (167b)

To a solution of Intermediate-167 (0.4 g, 1.31 mmol) in 1,4-dioxane was added 4 N HCl (2 mL) in 1,4-dioxane at 0° C. followed by stirring at RT for 6 h. The mixture was concentrated and triturated with diethyl ether to obtain the title compound as white solid (0.20 g) which was used in next step as such.

### d) N-((2-Bromo-6-methylpyridin-4-yl)methyl)methanesulfonamide (Intermediate-168)

To a solution of compound 167b (0.3 g, 1.49 mmol) and triethylamine (0.45 g, 4.47 mmol) in DCM (5 mL) was added MsCl (0.21 g, 1.79 mmol) followed by stirring at RT for 12 h. The mixture was diluted with water and extracted with DCM. The combined organic layer was washed with brine, dried over anhydrous sodium sulphate, filtered, concentrated, and purified by silica-gel flash column chromatography using 20% ethyl acetate in hexane as eluent to afford the title compound as a liquid (0.40 g, 96%). LCMS: 279.0 [M+H]⁺.

The following intermediates were prepared according to the procedure described for Intermediate-151 (procedure A′), Intermediate 152 (procedure B′) Intermediates 103, 104 and 155 (procedure C′) with appropriate starting materials, variations in reactants, quantities of reagents, coupling methods and solvents. The characterization data of the compounds are also summarized in the Table.

| | | | LCMS |
|---|---|---|---|
| No. | Structure | Procedure | [M + H]⁺ |
| Int-169 | 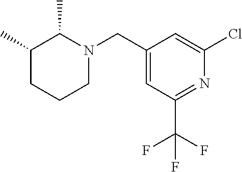 [SMILES: C[C@H]1CCCN(Cc2cc(Cl)nc(C(F)(F)F)c2)[C@H]1C] | B′ | 307.1 |
| Int-170 | 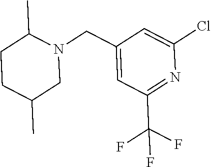 [SMILES: CC1CCC(C)N(Cc2cc(Cl)nc(C(F)(F)F)c2)C1] | B′ | 307.2 |
| Int-172 | 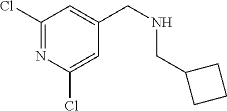 [SMILES: Clc1cc(CNCC2CCC2)cc(Cl)n1] | A′ | 244.8 |
| Int-174 | 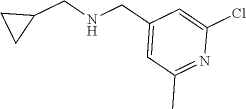 | A′ | 211.1 |
| Int-175 | 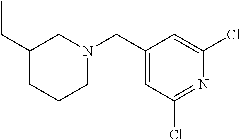 [SMILES: CCC1CCCN(Cc2cc(Cl)nc(Cl)c2)C1] | C′ | 272.95 |
| Int-176 | 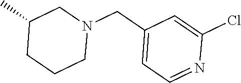 | B′ | 225.2 |
| Int-178 | 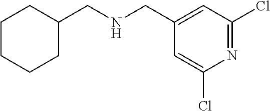 [SMILES: Clc1cc(CNCC2CCCCC2)cc(Cl)n1] | A′ | 273.1 |
| Int-179 | 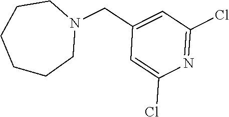 [SMILES: Clc1cc(CN2CCCCCC2)cc(Cl)n1] | A′ | 259.1 |
| Int-180 | 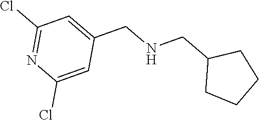 [SMILES: Clc1cc(CNCC2CCCC2)cc(Cl)n1] | B′ | 259.1 |
| Int-181 | 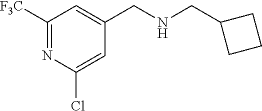 [SMILES: FC(F)(F)c1cc(CNCC2CCC2)cc(Cl)n1] | A′ | 278.95 |
| Int-182 | 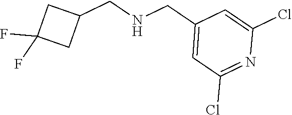 [SMILES: FC1(F)CC(CNCc2cc(Cl)nc(Cl)c2)C1] | C′ | 281.0 |
| Int-183 | 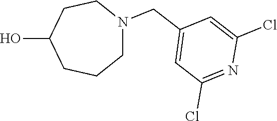 [SMILES: OC1CCCN(Cc2cc(Cl)nc(Cl)c2)CC1] | B′ | 275.1 |
| Int-184 | 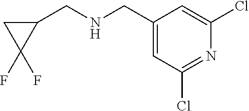 [SMILES: FC1(F)CC1CNCc1cc(Cl)nc(Cl)c1] | B′ | 268.85 [M + 2 + H]⁺ |
| Int-185 | 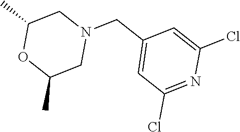 [SMILES: C[C@@H]1CN(Cc2cc(Cl)nc(Cl)c2)C[C@@H](C)O1] | A′ | 275.0 |
| Int-186 | 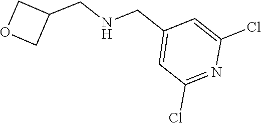 [SMILES: Clc1cc(CNCC2COC2)cc(Cl)n1] | A′ | 247.1 |
| Int-187 | 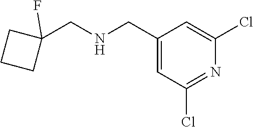 [SMILES: FC1(CNCc2cc(Cl)nc(Cl)c2)CCC1] | C′ | 263.0 |
| Int-188 | 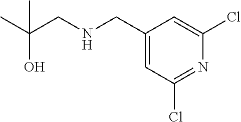 | A′ | 249.1 |
| Int-189 | 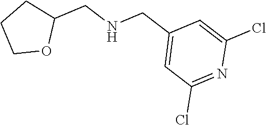 [SMILES: Clc1cc(CNCC2CCCO2)cc(Cl)n1] | A′ | 261.0 |
| Int-190 | 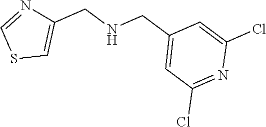 [SMILES: Clc1cc(CNCc2cscn2)cc(Cl)n1] | A′ | 274.0 |
| Int-191 | 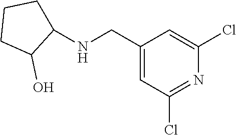 [SMILES: OC1CCCC1NCc1cc(Cl)nc(Cl)c1] | A′ | 261.0 |
| Int-192 | 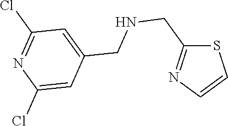 [SMILES: Clc1cc(CNCc2nccs2)cc(Cl)n1] | A′ | 274.0 |
| Int-193 | 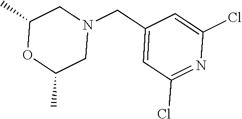 [SMILES: C[C@@H]1CN(Cc2cc(Cl)nc(Cl)c2)C[C@H](C)O1] | A′ | 275.1 |
| Int-194 | 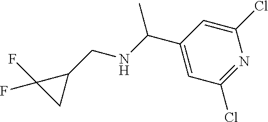 [SMILES: CC(NCC1CC1(F)F)c1cc(Cl)nc(Cl)c1] | C′ | 281.0 |
| Int-195 | 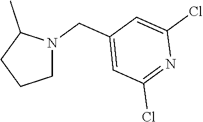 | A′ | 245.0 |
| Int-196 | 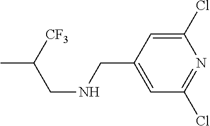 [SMILES: CC(CNCc1cc(Cl)nc(Cl)c1)C(F)(F)F] | C′ | 287.0 |
| Int-197 | 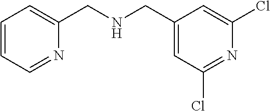 [SMILES: Clc1cc(CNCc2ccccn2)cc(Cl)n1] | C′ | 268.1 |
| Int-198 | 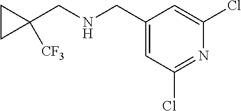 [SMILES: FC(F)(F)C1(CNCc2cc(Cl)nc(Cl)c2)CC1] | A′ | 299.0 |
| Int-199 | 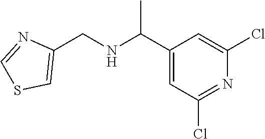 [SMILES: CC(NCc1cscn1)c1cc(Cl)nc(Cl)c1] | C′ | 288.0 |
| Int-200 | 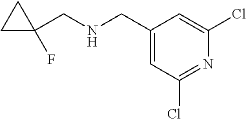 [SMILES: FC1(CNCc2cc(Cl)nc(Cl)c2)CC1] | C′ | 249.0 |
| Int-201 | 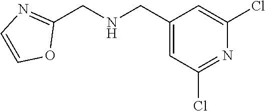 [SMILES: Clc1cc(CNCc2ncco2)cc(Cl)n1] | C′ | 258.0 |
| Int-202 | 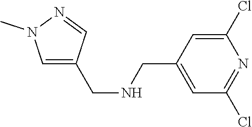 [SMILES: Cn1cc(CNCc2cc(Cl)nc(Cl)c2)cn1] | C′ | 271.20 |
| Int-203 | 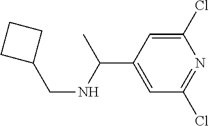 [SMILES: CC(NCC1CCC1)c1cc(Cl)nc(Cl)c1] | C′ | 259.2 |
| Int-204 | 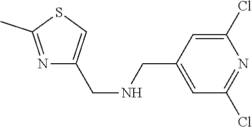 [SMILES: Cc1nc(CNCc2cc(Cl)nc(Cl)c2)cs1] | A′ | 288.0 |
| Int-205 | 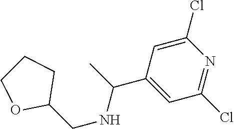 [SMILES: CC(NCC1CCCO1)c1cc(Cl)nc(Cl)c1] | C′ | 275.0 |
| Int-206 | 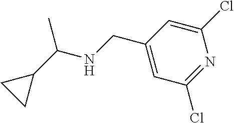 | C′ | 245.0 |
| Int-207 | 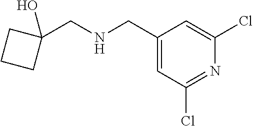 [SMILES: OC1(CNCc2cc(Cl)nc(Cl)c2)CCC1] | C′ | 261.0 |
| Int-208 | 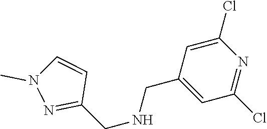 [SMILES: Cn1ccc(CNCc2cc(Cl)nc(Cl)c2)n1] | C′ | 271.1 |
| Int-209 | 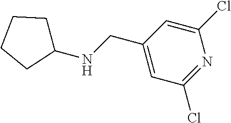 [SMILES: Clc1cc(CNC2CCCC2)cc(Cl)n1] | A′ | 245.1 |
| Int-210 | 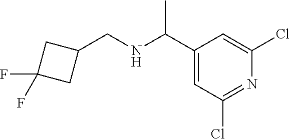 [SMILES: CC(NCC1CC(F)(F)C1)c1cc(Cl)nc(Cl)c1] | C′ | 295.0 |
| Int-211 | 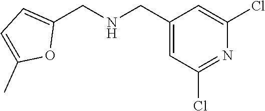 [SMILES: Cc1ccc(CNCc2cc(Cl)nc(Cl)c2)o1] | A′ | 271.1 |
| Int-212 | 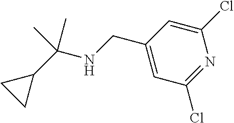 [SMILES: CC(C)(NCc1cc(Cl)nc(Cl)c1)C1CC1] | C′ | 259.1 |
| Int-213 | 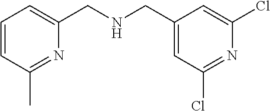 [SMILES: Cc1cccc(CNCc2cc(Cl)nc(Cl)c2)n1] | C′ | 282.1 |
| Int-214 | 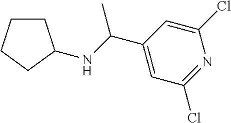 [SMILES: CC(NC1CCCC1)c1cc(Cl)nc(Cl)c1] | C′ | 259.1 |
| Int-215 | 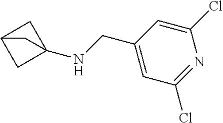 [SMILES: Clc1cc(CNC23CC(C2)C3)cc(Cl)n1] | C′ | 243.1 |
| Int-216 | 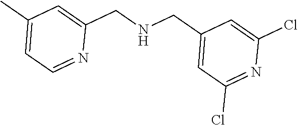 [SMILES: Cc1ccnc(CNCc2cc(Cl)nc(Cl)c2)c1] | C′ | 281.90 |
| Int-217 | 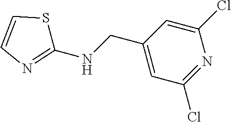 [SMILES: Clc1cc(CNc2nccs2)cc(Cl)n1] | A′ | 260.0 |
| Int-218 | 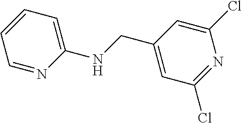 [SMILES: Clc1cc(CNc2ccccn2)cc(Cl)n1] | A′ | 254.0 |
| Int-219 | 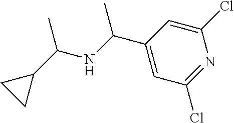 [SMILES: CC(NC(C)C1CC1)c1cc(Cl)nc(Cl)c1] | C′ | 259.1 |
| Int-220 | 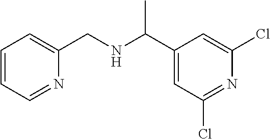 [SMILES: CC(NCc1ccccn1)c1cc(Cl)nc(Cl)c1] | C′ | 281.95 |
| Int-221 | 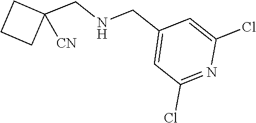 [SMILES: N#CC1(CNCc2cc(Cl)nc(Cl)c2)CCC1] | C′ | 270.1 |
| Int-222 | 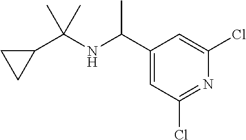 [SMILES: CC(NC(C)(C)C1CC1)c1cc(Cl)nc(Cl)c1] | C′ | 275.1 [M + 2 + H]⁺ |
| Int-223 | 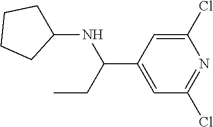 [SMILES: CCC(NC1CCCC1)c1cc(Cl)nc(Cl)c1] | C′ | 273.0 |
| Int-224 | 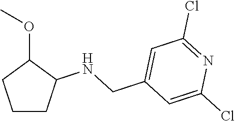 [SMILES: COC1CCCC1NCc1cc(Cl)nc(Cl)c1] | C′ | 275.1 |
| Int-225 | 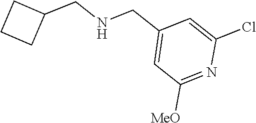 [SMILES: COc1cc(CNCC2CCC2)cc(Cl)n1] | B′ | 241.1 |
| Int-226 | 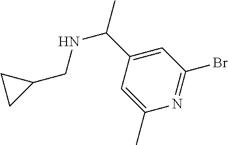 | C′ | 271.0 [M + 2 + H]⁺ |
| Int-227 | 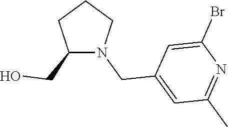 [SMILES: Cc1cc(CN2CCC[C@@H]2CO)cc(Br)n1] | A′ | 287.0 [M + 2 + H]⁺ |
| Int-228 | 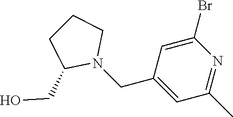 [SMILES: Cc1cc(CN2CCC[C@H]2CO)cc(Br)n1] | A′ | 287.1 [M + 2 + H]⁺ |
| Int-229 | 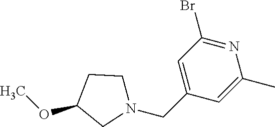 | A′ | 285.10 |
| Int-230 | 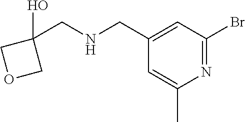 [SMILES: Cc1cc(CNCC2(O)COC2)cc(Br)n1] | A′ | 289.0 [M + 2 + H]⁺ |
| Int-231 | 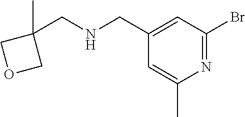 [SMILES: Cc1cc(CNCC2(C)COC2)cc(Br)n1] | A′ | 285.1 |
| Int-232 | 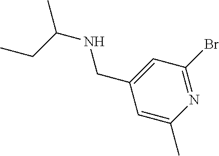 | A′ | 258.1 |
| Int-233 | 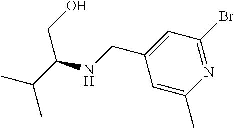 [SMILES: Cc1cc(CN[C@H](CO)C(C)C)cc(Br)n1] | A′ | 289.1 [M + 2 + H]⁺ |
| Int-234 | 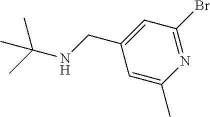 | A′ | 259.10 [M + 2 + H]⁺ |
| Int-235 | 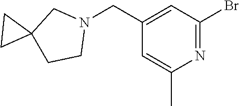 [SMILES: Cc1cc(CN2CCC3(CC3)C2)cc(Br)n1] | B′ | 281.10 |
| Int-236 | 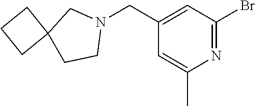 [SMILES: Cc1cc(CN2CCC3(CCC3)C2)cc(Br)n1] | B′ | 295.0 |
| Int-237 | 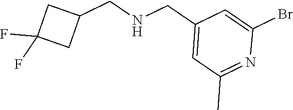 [SMILES: Cc1cc(CNCC2CC(F)(F)C2)cc(Br)n1] | B′ | 307.0 [M + 2 + H]⁺ |
| Int-238 | 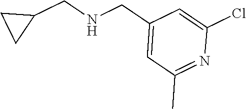 | A′ | 211.20 |
| Int-239 | 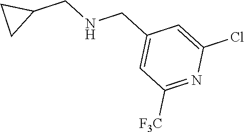 [SMILES: FC(F)(F)c1cc(CNCC2CC2)cc(Cl)n1] | A′ | 265.1 |
| Int-240 | 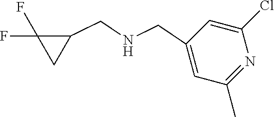 [SMILES: Cc1cc(CNCC2CC2(F)F)cc(Cl)n1] | C′ | 247.00 |
| Int-241 | 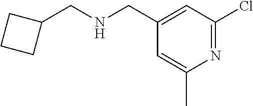 | B′ | 225.10 |
| Int-242 | 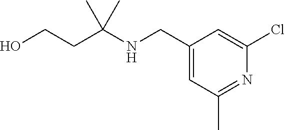 [SMILES: Cc1cc(CNC(C)(C)CCO)cc(Cl)n1] | A′ | 243.10 |
| Int-243 | 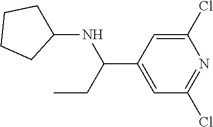 [SMILES: CCC(NC1CCCC1)c1cc(Cl)nc(Cl)c1] | C′ | 239.10 |
| Int-244 | 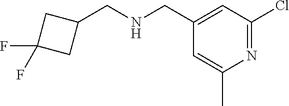 [SMILES: Cc1cc(CNCC2CC(F)(F)C2)cc(Cl)n1] | B′ | 261.10 |
| Int-246 | 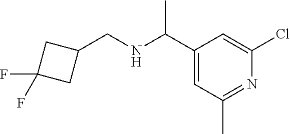 [SMILES: Cc1cc(C(C)NCC2CC(F)(F)C2)cc(Cl)n1] | C′ | 275.10 |

### Intermediate-248. 6-(2-([1,2,4]Triazolo[4,3-a]pyridin-3-yl)-4-chlorophenyl)-isoindolin-1-one

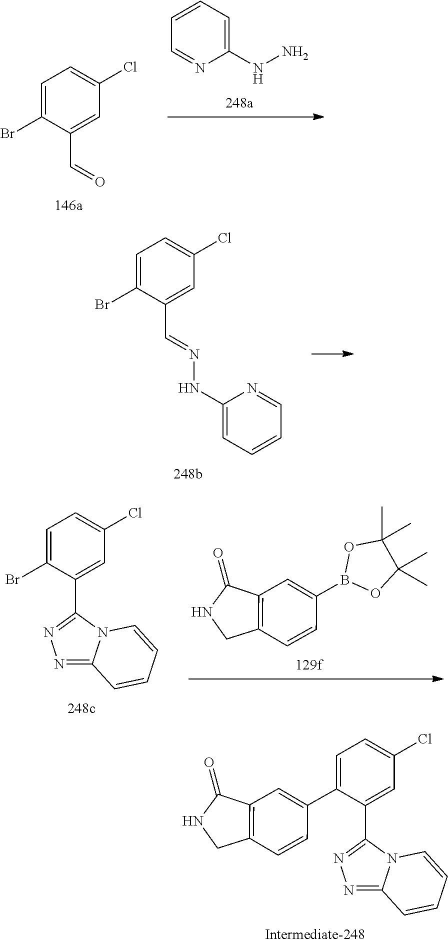

### a) (E)-2-(2-(2-Bromo-5-chlorobenzylidene)hydrazineyl)pyridine (248b)

To a solution of 2-bromo-5-chlorobenzaldehyde (5 g, 22.78 mmol) and 2-hydrazineylpyridine (2.74 g, 25.06 mmol) in MeOH (30 mL) was added AcOH (0.08 g, 1.38) followed by stirring at RT for 16 h. The precipitated solid was filtered, triturated with MeOH and dried to obtain the title compound (6.8 g). LCMS: 310.0 [M+H]⁺.

### b) 3-(2-Bromo-5-chlorophenyl)-[1,2,4]triazolo[4,3-a]pyridine (248c)

To a solution of compound 248b (6 g, 19.32 mmol) in DCM (20 mL) was added [Bis(trifluoroacetoxy)iodo]benzene (9.15 g, 21.27 mmol) at 0° C. The mixture was slowly warmed to RT and stirred for 16 h. The mixture was concentrated and purified by silica-gel flash column chromatography using 65% ethyl acetate in hexane as eluent to obtain titled compound (2.2 g, 37%). LCMS: 307.9 [M+H]⁺.

### c) 6-(2-([1,2,4]Triazolo[4,3-a]pyridin-3-yl)-4-chlorophenyl)isoindolin-1-one (Intermediate-248)

The compound was prepared according to the procedure described for Intermediate-93 with appropriate variations in reactants, quantities of reagents, and solvents. LCMS: 361.1 [M+H]⁺.

### Intermediate-249. 3-(1-(4-Methyl-4H-1,2,4-triazol-3-yl)propan-2-yl)-6,7-dihydro-5H-pyrrolo[3,4-b]pyridin-5-one

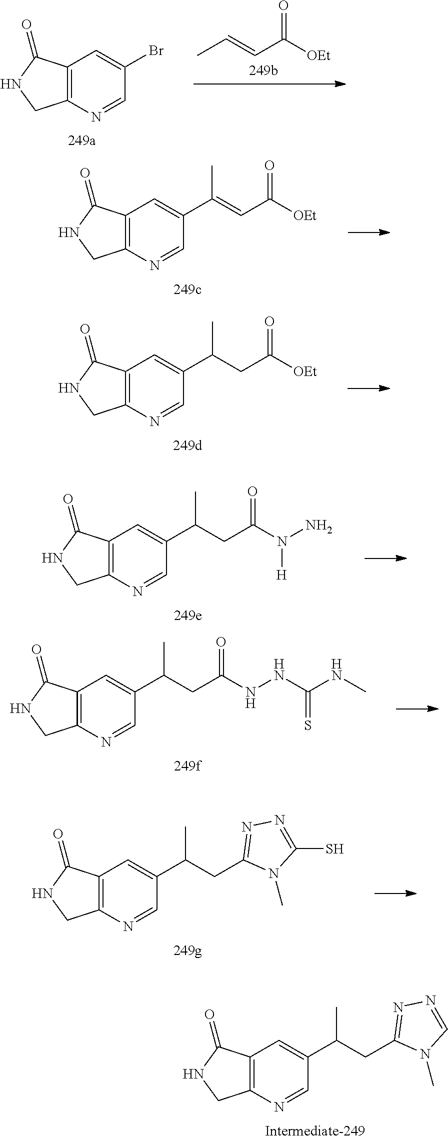

### a) Ethyl (E)-3-(5-oxo-6,7-dihydro-5H-pyrrolo[3,4-b]pyridin-3-yl)but-2-enoate (249c)

To a degassed solution of 3-bromo-6,7-dihydro-5H-pyrrolo[3,4-b]pyridin-5-one (4.5 g, 21.12 mmol) (prepared according to WO 2021/143927), triethylamine (3.21 g, 31.68 mmol) and ethyl (E)-but-2-enoate (7.23 g, 63.37 mmol) in DMF (50 ml) was added palladium acetate (0.24 g, 1.05 mmol) and tris(2-methylphenyl)phosphane (0.64 g, 2.11 mmol) followed by stirring at 90° C. for 16 h. The mixture was diluted with water and extracted with EtOAc. The combined organic layer was treated as in example Intermediate-124 step d using 5% MeOH in DCM as eluent to afford the title compound (3.5 g, 67.3%). LCMS: 245.0 [M−H]⁻.

### b) Ethyl 3-(5-oxo-6,7-dihydro-5H-pyrrolo[3,4-b]pyridin-3-yl)butanoate (249d)

The compound was prepared according to the procedure described for Intermediate-127c with appropriate variations in reactants, quantities of reagents, and solvents. LCMS: 249.1 [M+H]⁺.

### c) 3-(5-Oxo-6,7-dihydro-5H-pyrrolo[3,4-b]pyridin-3-yl)butanehydrazide (249e)

The compound was prepared according to the procedure described for Intermediate-if with appropriate variations in reactants, quantities of reagents, and solvents. LCMS: 235.1 [M+H]⁺.

### d) N-Methyl-2-(3-(5-oxo-6,7-dihydro-5H-pyrrolo[3,4-b]pyridin-3-yl)butanoyl)-hydrazine-1-carbothioamide (249f)

The compound was prepared according to the procedure described for Intermediate-1g with appropriate variations in reactants, quantities of reagents, and solvents. LCMS: 308.1 [M+H]⁺.

### e) 3-(1-(5-Mercapto-4-methyl-4H-1,2,4-triazol-3-yl)propan-2-yl)-6,7-dihydro-5H-pyrrolo[3,4-b]pyridin-5-one (249g)

The compound was prepared according to the procedure described for Intermediate-1h with appropriate variations in reactants, quantities of reagents, and solvents. LCMS: 290.1 [M+H]⁺.

### f) 3-(1-(4-Methyl-4H-1,2,4-triazol-3-yl)propan-2-yl)-6,7-dihydro-5H-pyrrolo[3,4-b]pyridin-5-one (Intermediate-249)

The compound was prepared according to the procedure described for Intermediate-1 (last step) with appropriate variations in reactants, quantities of reagents, and solvents. LCMS: 258.1 [M+H]⁺.

### Example 1. (S)-6-(3-((4-Methyl-4H-1,2,4-triazol-3-yl)methyl)oxetan-3-yl)-2-(3-((3-methylpiperidin-1-yl)methyl)-5-(trifluoromethyl)phenyl)isoindolin-1-one (Compound 1)

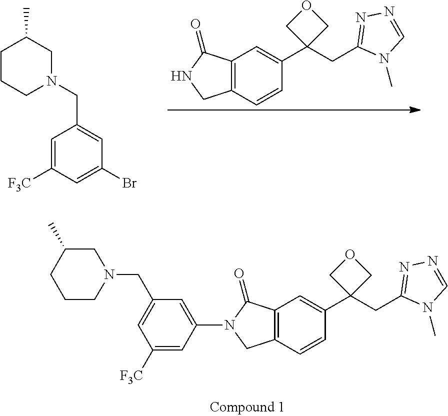 [SMILES: C[C@H]1CCCN(Cc2cc(Br)cc(C(F)(F)F)c2)C1 ; Cn1cnnc1CC1(c2ccc3c(c2)C(=O)NC3)COC1 ; C[C@H]1CCCN(Cc2cc(N3Cc4ccc(C5(Cc6nncn6C)COC5)cc4C3=O)cc(C(F)(F)F)c2)C1]

Conditions A: To a degassed solution of 6-(3-((4-methyl-4H-1,2,4-triazol-3-yl)-methyl)oxetan-3-yl)isoindolin-1-one (0.10 g, 0.35 mmol), (S)-1-(3-bromo-5-(trifluoro methyl)benzyl)-3-methylpiperidine (0.142 g, 0.42 mmol), cesium carbonate (0.23 g, 0.70 mmol), potassium iodide (0.12 g, 0.70 mmol), and N, N′-dimethyl ethylene diamine (40.0 μL, 0.35 mmol) in DMF (4.0 mL) was added copper(I) iodide (6.67 mg, 0.035 mmol) followed by stirring at 90° C. for 16 h. The mixture was cooled to RT, filtered through celite pad followed by washing the celite pad with ethyl acetate. The filtrate was diluted with water and extracted with ethyl acetate. The combined organic layers were dried over anhydrous sodium sulphate, filtered, and concentrated. The obtained crude product was purified by silica-gel preparative thin layer chromatography (TLC) using 5% MeOH in DCM as eluent to afford the title compound as an off-white solid (0.025 g, 13%). LCMS: 540.2 [M+H]⁺. ¹H NMR (400 MHz, CD3OD) δ: 8.25 (brs, 2H), 8.05 (s, 1H), 7.58 (d, 1H), 7.50 (s, 1H), 7.43 (s, 1H), 7.33 (d, 1H), 5.06 (s, 2H), 4.97 (d, 2H), 4.90 (d, 2H), 3.58 (s, 2H), 3.55 (s, 2H), 3.02 (s, 3H), 2.80-2.70 (m, 2H), 1.95-1.89 (m, 1H), 1.73-1.45 (m, 5H), 0.95-0.85 (m, 1H), 0.83 (d, 3H).

### Example 2. ((S)-6-(3-((4-Methyl-4H-1,2,4-triazol-3-yl)methyl)oxetan-3-yl)-2-(4-((3-methylpiperidin-1-yl)methyl)-6-(trifluoromethyl)pyridin-2-yl)isoindolin-1-one (Compound 2)

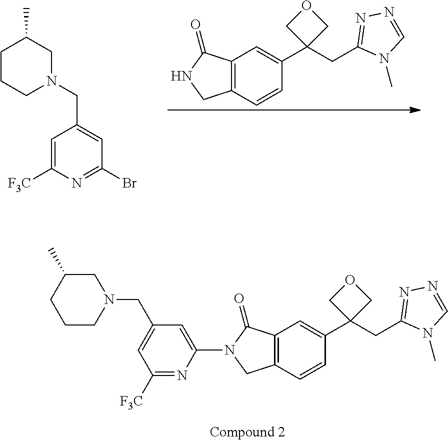 [SMILES: C[C@H]1CCCN(Cc2cc(Br)nc(C(F)(F)F)c2)C1 ; Cn1cnnc1CC1(c2ccc3c(c2)C(=O)NC3)COC1 ; C[C@H]1CCCN(Cc2cc(N3Cc4ccc(C5(Cc6nncn6C)COC5)cc4C3=O)nc(C(F)(F)F)c2)C1]

Conditions B: To a degassed solution of (S)-2-chloro-4-((3-methylpiperidin-1-yl)methyl)-6-(trifluoromethyl)pyridine (0.10 g, 0.34 mmol), 6-(3-((4-methyl-4H-1,2,4-triazol-3-yl)methyl)oxetan-3-yl)isoindolin-1-one (0.10 g, 0.34 mmol), dicesium carbonate (0.33 g, 1.03 mmol) and Xantphos (0.04 g, 0.07 mmol) in DMSO (1.0 ml) was added Pd(OAc)₂ (0.008 g, 0.030 mmol) and heated at 100° C. for 1 h. The mixture was cooled to RT, passed through celite bed followed by washing the celite pad with 10% methanol in DCM. The filtrate was diluted with water and extracted with 10% methanol in DCM. The combined organic layers were washed with brine, dried over anhydrous sodium sulphate, filtered, concentrated, and purified by preparative TLC using 7% methanol in DCMe as eluent to afford the title compound (0.0198 g, 10.71%); LCMS: 542.2 [M+H]⁺. ¹H NMR (400 MHz, CD3OD) δ: 8.81 (s, 1H), 8.20 (s, 1H), 7.64 (dd, 1H), 7.60 (d, 1H), 7.50 (d, 1H), 7.27 (dd, 1H), 5.16 (s, 2H), 5.13 (d, 2H), 5.09 (d, 2H), 3.70 (s, 2H), 3.67 (s, 2H), 2.96 (s, 3H), 2.90-2.80 (m, 2H), 2.10-2.05 (m, 1H), 1.76-1.69 (m, 5H), 1.00-0.90 (m, 1H), 0.90 (d, 3H).

The following compounds were prepared according to the procedure described for Compound 1 (=conditions A) and Compound 2 (=conditions B) with appropriate variations in reactants, quantities of reagents, coupling methods and solvents. The characterization data of the compounds are summarized in the table.

| | Structure, starting material and | |
|---|---|---|
| No. | reaction conditions | Characterization data |
| 3 | 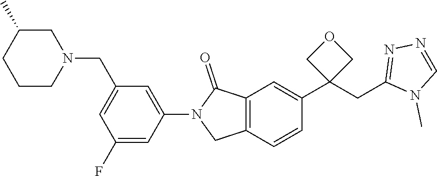 [SMILES: C[C@H]1CCCN(Cc2cc(F)cc(N3Cc4ccc(C5(Cc6nncn6C)COC5)cc4C3=O)c2)C1] | LCMS: 490.20 [M + H]⁺. ¹H NMR (400 MHz, CD₃OD): δ 8.17 (s, 1H), 7.79- 7.75 (m, 1H), 7.64 (brs, 1H), 7.57 (d, 1H), 7.44 (s, 1H), 7.21 (dd, 1H), 6.97 (d, 1H), 5.09 (d, 2H), 5.04 (d, 2H), 4.96 (s, 2H), 3.67 (s, 4H), 3.00-2.85 (m, 2H), 2.92 (s, 3H), 2.05-1.98 (m, 1H), 1.75-1.55 (m, 5H), 1.00-0.90 (m, 1H), 0.87 (d, 3H). |
| | Starting materials: Int-1 and Int-39 | |
| | Reaction conditions: A | |
| 4 | 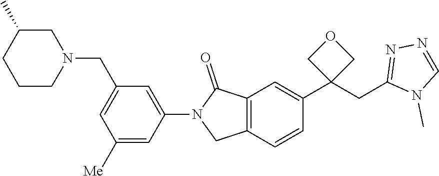 [SMILES: Cc1cc(CN2CCC[C@H](C)C2)cc(N2Cc3ccc(C4(Cc5nncn5C)COC4)cc3C2=O)c1] | LCMS : 486.30 [M + H]⁺. ¹H NMR (400 MHz, CD₃OD): δ 8.17 (s, 1H), 7.71 (brs, 1H), 7.62 (s, 1H), 7.57 (d, 1H), 7.43 (s, 1H), 7.20 (dd, 1H), 7.07 (s, 1H), 5.09 (d, 2H), 5.04 (d, 2H), 4.97 (s, 2H), 3.75 (brs, 2H), 3.67 (s, 2H), 3.08-2.97 (m, 2H), 2.94 (s, 3H), 2.40 (s, 3H), 2.04-1.88 (m, 1H), 1.80-1.55 (m, 5H), 1.03-0.90 (m, 1H), 0.88 (d, 3H). |
| | Starting materials: Int-1 and Int-40 | |
| | Reaction conditions: A | |
| 5 | 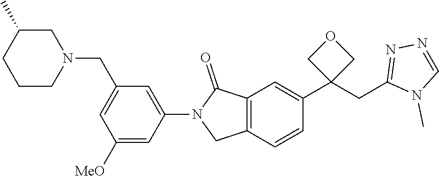 [SMILES: COc1cc(CN2CCC[C@H](C)C2)cc(N2Cc3ccc(C4(Cc5nncn5C)COC4)cc3C2=O)c1] | LCMS: 502.20 [M + H]⁺. ¹H NMR (400 MHz, CD₃OD): δ 8.17 (s, 1H), 7.56 (d, 1H), 7.50 (t, 1H), 7.41 (d, 1H), 7.34 (s, 1H), 7.18 (dd, 1H), 6.80 (s, 1H), 5.09 (d, 2H), 5.04 (d, 2H), 4.95 (s, 2H), 3.83 (s, 3H), 3.66 (s, 2H), 3.58 (s, 2H), 2.98-2.85 (m, 2H), 2.91 (s, 3H), 2.07-1.97 (m, 1H), 1.80-1.50 (m, 5H), 0.95-0.88 (m, 1H), 0.86 (d, 3H). |
| | Starting materials: Int-1 and Int-41 | |
| | Reaction conditions: A | |
| 6 | 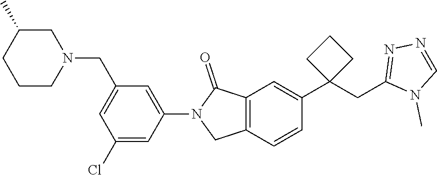 | LCMS: 507.20 [M + H]⁺. ¹H NMR (400 MHz, CD₃OD): δ 8.17 (s, 1H), 7.98 (t, 1H), 7.75 (brs, 1H), 7.57 (d, 1H), 7.44 (s, 1H), 7.23-7.19 (m, 2H), 5.09 (d, 2H), 5.04 (d, 2H), 4.96 (s, 2H), 3.66 (s, 2H), 3.61 (brs, 2H), 2.96-2.84 (m, 2H), 2.93 (s, 3H), 2.08-1.98 (m, 1H), 1.77-1.55 (m, 5H), 0.98-0.89 (m, 1H), 0.86 (d, 3H). |
| | Starting materials: Int-1 and Int-42 | |
| | Reaction conditions: A | |
| 7 | 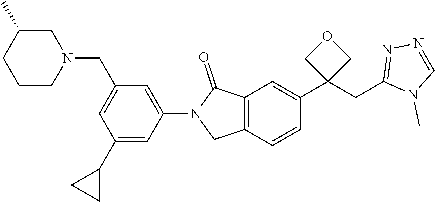 [SMILES: C[C@H]1CCCN(Cc2cc(C3CC3)cc(N3Cc4ccc(C5(Cc6nncn6C)COC5)cc4C3=O)c2)C1] | LCMS: 512.30 [M + H]⁺. ¹H NMR (400 MHz, CD₃OD): δ 8.17 (s, 1H), 7.57 (brs, 1H), 7.54 (d, 2 H), 7.42 (d, 1H), 7.18 (dd, 1H), 6.94 (s, 1H), 5.09 (d, 2H), 5.04 (d, 2H), 4.95 (s, 2H), 3.66 (s, 2H), 3.61 (brs, 2H), 2.98-2.88 (m, 2H), 2.92 (s, 3H), 1.98-1.93 (m, 1H), 1.78-1.58 (m, 6H), 1.05-0.95 (m, 2H), 0.94-0.90 (m, 1H), 0.86 (d, 3H), 0.78- 0.70 (m, 2H). |
| | Starting materials: Int-1 and Int-14 | |
| | Reaction conditions: A | |
| 8 | 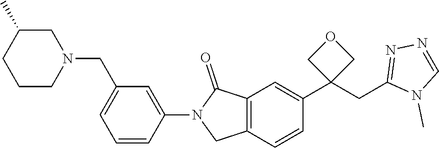 [SMILES: C[C@H]1CCCN(Cc2cccc(N3Cc4ccc(C5(Cc6nncn6C)COC5)cc4C3=O)c2)C1] | LCMS: 472.05 [M + H]+. ¹H NMR (400 MHz, CD₃OD): δ 8.17 (s, 1H), 7.84 (s, 1H), 7.77 (dd, 1 H), 7.57 (d, 1H), 7.42 (s, 1H), 7.39 (d, 1H), 7.20 (s, 1H), 7.18 (dd, 1H), 5.09 (d, 2H), 5.04 (d, 2H), 4.97 (s, 2H), 3.66 (s, 2H), 3.62 (s, 2H), 2.95-2.85 (m, 2H), 2.92 (s, 3H), 2.07-1.90 (m, 1H), 1.87-1.67 (m, 4H), 1.62-1.58 (m, 1H), 0.95-0.86 (m, 1H), 0.85 (d, 3H). |
| | Starting materials: Int-1 and Int-43 | |
| | Reaction conditions: A | |
| 9 | 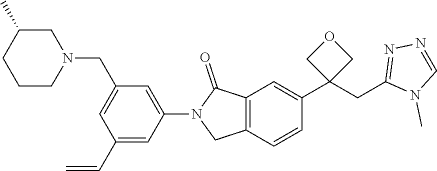 [SMILES: C=Cc1cc(CN2CCC[C@H](C)C2)cc(N2Cc3ccc(C4(Cc5nncn5C)COC4)cc3C2=O)c1] | LCMS: 498.40 [M + H]⁺. ¹H NMR (400 MHz, CD₃OD): δ 8.17 (s, 1H), 7.88 (brs, 1H), 7.74 (s, 1H), 7.57 (d, 1H), 7.43 (d, 1H), 7.30 (s, 1H), 7.19 (dd, 1H), 6.82-6.72 (m, 1H), 5.85 (d, 1H), 5.30 (d, 1H), 5.09 (d, 2H), 5.05 (d, 2H), 4.98 (s, 2H), 3.67 (s, 2H), 3.62 (brs, 2H), 2.98-2.85 (m, 2H), 2.92 (s, 3H), 2.10-2.00 (m, 1H), 1.75- 1.55 (m, 5H), 0.95-0.85 (m, 1H), 0.86 (d, 3H). |
| | Starting materials: Int-1 and Int-10 | |
| | Reaction conditions: A | |
| 10 | 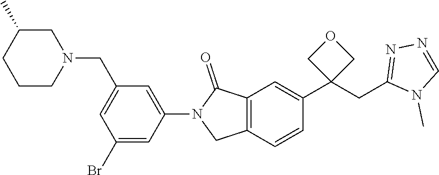 [SMILES: C[C@H]1CCCN(Cc2cc(Br)cc(N3Cc4ccc(C5(Cc6nncn6C)COC5)cc4C3=O)c2)C1] | LCMS: 551.10 [M + H]⁺. ¹H NMR (400 MHz, CD₃OD): δ 8.17 (s, 1H), 8.14 (t, 1H), 7.92 (s, 1H), 7.58 (d, 1H), 7.44 (d, 1H), 7.23 (dd, 2H), 5.08 (d, 2H), 5.02 (d, 2H), 4.96 (s, 2H), 3.86 (s, 2H), 3.70 (s, 2H), 3.15-3.05 (m, 2H), 2.95 (s, 3H), 2.13-2.05 (m, 1H) , 1.82-1.72 (m, 4H), 1.70-1.60 (m, 1H), 1.05-0.95 (m, 1H), 0.87 (d, 3H). |
| | Starting materials: Int-1 and Int-44 | |
| | Reaction conditions: A | |
| 11 | 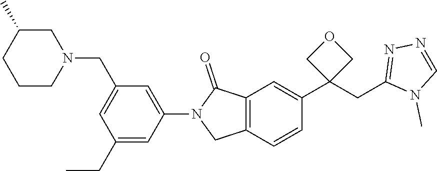 [SMILES: CCc1cc(CN2CCC[C@H](C)C2)cc(N2Cc3ccc(C4(Cc5nncn5C)COC4)cc3C2=O)c1] | LCMS: 500.2 [M + H]⁺. ¹H NMR (400 MHz, CD₃OD): δ 8.19 (brs, 1H), 7.64 (s, 1H), 7.62 (s, 1H), 7.56 (d, 1H), 7.42 (s, 1H), 7.18 (d, 1H), 7.06 (s, 1H), 5.09 (d, 2H), 5.04 (d, 2H), 4.95 (s, 2H), 3.67 (s, 2H), 3.56 (s, 2H), 2.92 (s, 3H), 2.91-2.85 (m, 2H), 2.69 (q, 2H), 2.02- 1.94 (m, 1H), 1.75-1.55 (m, 5H), 1.26 (t, 3H), 0.96-0.86 (m, 1H), 0.85 (d, 3H). |
| | Starting materials: Int-1 and Int-12 | |
| | Reaction conditions: A | |
| 12 | 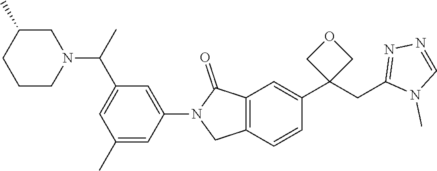 [SMILES: Cc1cc(C(C)N2CCC[C@H](C)C2)cc(N2Cc3ccc(C4(Cc5nncn5C)COC4)cc3C2=O)c1] | LC-MS: 500.2 [M + H]⁺. ¹H NMR (DMSO-d6, 400 MHz): δ 8.21 (s, 1H), 7.67 (bs, 1H), 7.59-7.52 (m, 2H), 7.45 (s, 1H), 7.23 (d, 1H), 6.93 (s, 1H), 5.05-4.95 (m, 4H), 4.91 (d, 2H), 3.57 (s, 2H), 3.44- 3.39 (m, 1H), 3.00 (s, 3H), 2.91-2.78 (m, 1H), 2.33 (s, 3H), 1.89-1.78 (m, 1H), 1.68-1.55 (m, 3H), 1.55-1.49 (m, 2H), 1.33-1.26 (m, 3H), 1.26-1.20 (m, 1H), 0.84-0.75 (m, 3H). |
| | Starting materials: Int-1 and Int-27 | |
| | Reaction conditions: A | |
| 13 | 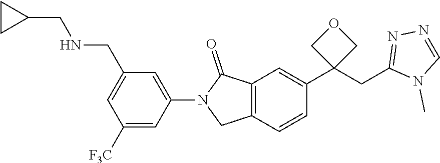 [SMILES: Cn1cnnc1CC1(c2ccc3c(c2)C(=O)N(c2cc(CNCC4CC4)cc(C(F)(F)F)c2)C3)COC1] | LCMS: 512.2 [M + H]⁺. ¹H NMR (400 MHz, CD₃OD): δ 8.33 (s, 1H), 8.21 (s, 1H), 8.04 (s, 1H), 7.63 (d, 1H), 7.55 (s, 1H), 7.50 (d, 1H), 7.25 (dd, 1H), 5.13 (d, 2H), 5.09 (d, 2H), 5.06 (s, 2H), 3.95 (s, 2H), 3.70 (s, 2H), 2.98 (s, 3H), 2.52 (d, 2H), 1.08-0.97 (m, 1H), 0.57-0.52 (m, 2H), 0.21-0.18 (m, 2H). |
| | Starting materials: Int-1 and Int-45 | |
| | Reaction conditions: A | |
| 14 | 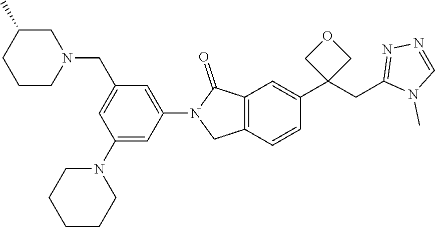 | LC-MS: 599.2 [M + HCO₂H]⁻. ¹H NMR (CD₃OD, 400 MHz): δ 8.17 (s, 1H), 7.55 (d, 1H), 7.49 (bs, 1H), 7.41 (s, 1H), 7.16 (dd, 1H), 7.14 (s, 1H), 6.84 (s, 1H), 5.09 (d, 2H), 5.04 (d, 2H), 4.94 (s, 2H), 3.66 (s, 2H), 3.51 (s, 2H), 3.22-3.18 (m, 4H), 2.90 (s, 3H), 2.86-2.84 (m, 2H), 1.99-1.94 (m, 1H), 1.72-1.66 (m, 6H), 1.65-1.60 (m, 5H), 0.95-0.88 (m, 1H), 0.85 (m, 3H). |
| | Starting materials: Int-1 and Int-6 | |
| | Reaction conditions: A | |
| 15 | 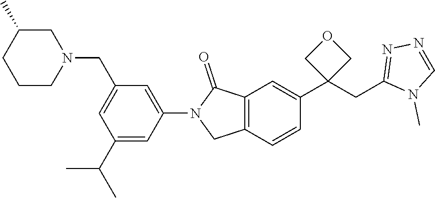 [SMILES: CC(C)c1cc(CN2CCC[C@H](C)C2)cc(N2Cc3ccc(C4(Cc5nncn5C)COC4)cc3C2=O)c1] | LC-MS: 514.3 [M + H]⁺. ¹H NMR (CD₃OD, 400 MHz): δ 8.21 (s, 1H), 7.73 (brs, 1H), 7.70 (brs, 1H), 7.61 (d, 1H), 7.47 (d, 1H), 7.24 (dd, 1H), 7.16 (s, 1H), 5.13 (d, 2H), 5.04 (d, 2H), 5.02 (s, 2H), 3.75 (bs, 2H), 3.71 (s, 2H), 3.10-2.97 (m, 2H), 2.96 (s, 3H), 2.10-2.02 (m, 1H), 1.82 -1.60 (m, 5H), 1.40-1.35 (m, 1H), 1.32 (d, 6H), 1.05-0.95 (m, 1H), 0.92 (d, 3H). |
| | Starting materials: Int-1 and Int-46 | |
| | Reaction conditions: A | |
| 16 | 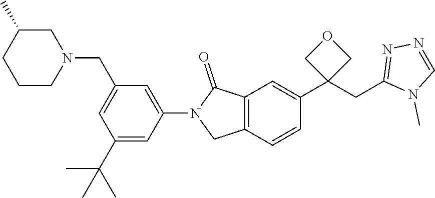 [SMILES: C[C@H]1CCCN(Cc2cc(N3Cc4ccc(C5(Cc6nncn6C)COC5)cc4C3=O)cc(C(C)(C)C)c2)C1] | LCMS: 528.30 [M + H]⁺. ¹H NMR (400 MHz, CD₃OD): δ 8.17 (s, 1H), 7.87 (t, 1H), 7.58-7.52 (m, 2H), 7.42 (d, 1H), 7.26 (s, 1H), 7.17 (dd, 1H), 5.09 (d, 2H), 5.05 (d, 4H), 4.98 (s, 2H), 3.67 (s, 2H), 3.56 (brs, 2H), 2.90 (s, 3H), 2.94-2.84 (m, 2H), 2.02-1.91 (m, 1H), 1.75-1.55 (m, 5H), 1.35 (d, 3H), 0.94-0.84 (m, 1H). |
| | Starting materials: Int-1 and Int-47 | |
| | Reaction conditions: A | |
| 17 | 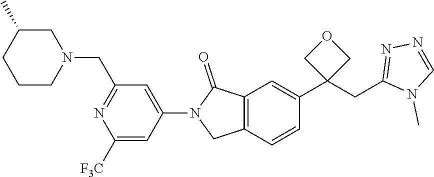 [SMILES: C[C@H]1CCCN(Cc2cc(N3Cc4ccc(C5(Cc6nncn6C)COC5)cc4C3=O)cc(C(F)(F)F)n2)C1] | LCMS: 541.2 [M + H]⁺. ¹H NMR (400 MHz, CD₃OD): δ 8.49 (d, 1H), 8.21 (brs, 1H), 8.16 (d, 1H), 7.65 (d, 1H), 7.54 (d, 1H), 7.31 (dd, 1H), 5.13 (d, 2H), 5.08 (d, 2H), 5.06 (s, 2H), 3.78 (s, 2H), 3.71 (s, 2H), 3.00 (s, 3H), 2.99- 2.90 (m, 2H), 2.20-2.10 (m, 1H), 1.80- 1.70 (m, 5H), 1.00-0.92 (m, 1H), 0.91 d, 3H). |
| | Starting materials: Int-1 and Int-16 | |
| | Reaction conditions: B | |
| 18 | 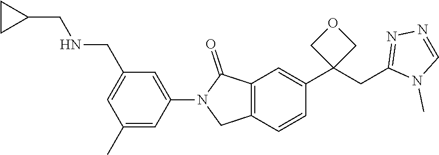 [SMILES: Cc1cc(CNCC2CC2)cc(N2Cc3ccc(C4(Cc5nncn5C)COC4)cc3C2=O)c1] | LCMS: 458.25 [M + H]⁺. ¹H NMR (400 MHz, DMSO-d6): δ 8.19 (s, 1H), 7.60 (bs, 1 H), 7.56 (d, 2H), 7.40 (s, 1H), 7.28 (dd, 1H), 6.98 (s, 1H), 4.98 (d, 2H), 4.94 (s, 2H), 4.89 (d, 2H), 3.55 (s, 2H), 2.97 (s, 3H), 2.37 (d, 2H), 2.32 (s, 3H), 0.97-0.81 (m, 1H), 0.45-0.35 (m, 2H), 0.12-0.06 (m, 2H). |
| | Starting materials: Int-1 and Int-5 | |
| | Reaction conditions: A | |
| 19 | 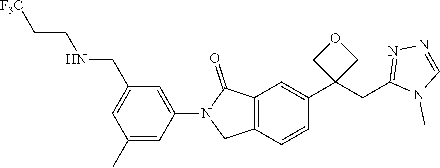 [SMILES: Cc1cc(CNCCC(F)(F)F)cc(N2Cc3ccc(C4(Cc5nncn5C)COC4)cc3C2=O)c1] | LCMS: 500.20 [M + H]⁺. ¹H NMR (400 MHz, DMSO-d6): δ 8.21 (s, 1H), 7.65 (s, 1H), 7.61 (s, 1H), 7.56 (d, 1H), 7.46 (d, 1H), 7.29 (dd, 1H), 6.98 (s, 1H), 4.99-4.90 (m, 4H), 4.91 (d, 2H), 3.70 (brs, 2H), 3.58 (s, 2H), 3.01 (s, 3H), 2.75-2.65 (m, 2H), 2.45-2.35 (m, 2H), 2.34 (s, 3H). |
| | Starting materials: Int-1 and Int-48 | |
| | Reaction conditions: A | |
| 20 | 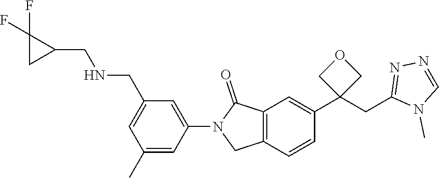 [SMILES: Cc1cc(CNCC2CC2(F)F)cc(N2Cc3ccc(C4(Cc5nncn5C)COC4)cc3C2=O)c1] | LCMS: 494.3 [M + H]⁺. ¹H NMR (DMSO-d6, 400 MHz): δ 8.22 (s, 1H), 7.65 (s, 1H), 7.61 (s, 1H), 7.56 (d, 1H), 7.46 (d, 1H), 7.29 (dd, 1H), 6.99 (s, 1H), 4.99-4.96 (m, 4H), 4.91 (d, 2H), 3.78-3.68 (m, 2H), 3.58 (s, 2H), 3.01 (s, 3H), 2.67- 2.57 (m, 2H), 2.34 (s, 3H), 1.89-1.83 (m, 1H), 1.57-1.48 (m, 1H), 1.23-1.14 (m, 1H). |
| | Starting materials: Int-1 and Int-49 | |
| | Reaction conditions: A | |
| 21 | 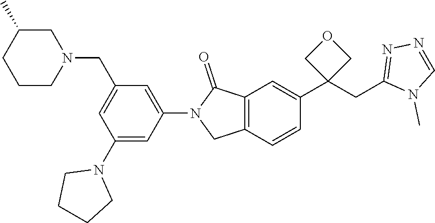 | LCMS: 585.3 [M + HCO₂H] . ¹H NMR (400 MHz, CDCl₃): δ 7.89 (s, 1H), 7.58 (d, 1 H), 7.37 (d, 1H), 7.24 (s, 1H), 6.98 (dd, 1H), 6.86 (s, 1H), 6.42 (s, 1H), 5.16 (d, 2H), 5.11 (d, 2H) 4.86 (s, 2H), 3.60 (s, 2H), 3.51 (s, 2H), 3.38-3.33 (m, 4H), 2.90 (s, 3H), 2.90-2.80 (m, 2H), 2.05-2.02 (m, 4H), 1.95-1.83 (m, 1H), 1.78-1.55 (m, 5H), 0.95-0.88 (m, 1H), 0.86 (d, 3H). |
| | Starting materials: Int-1 and Int-8 | |
| | Reaction conditions: A | |
| 22 | 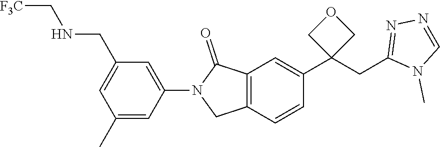 [SMILES: Cc1cc(CNCC(F)(F)F)cc(N2Cc3ccc(C4(Cc5nncn5C)COC4)cc3C2=O)c1] | LCMS: 486.10 [M + H]⁺. ¹H NMR (400 MHz, DMSO-d6): δ 8.29 (brs, 1H), 7.68 (d, 2H), 7.57 (d, 1H), 7.48 (d, 1H), 7.32 (dd, 1H), 7.02 (s, 1H), 4.98 (d, 4H), 4.91 (d, 2H), 3.88 (brs, 2H), 3.59 (s, 2H), 3.40-3.30 (m, 2H) 3.04 (s, 3H), 2.35 (s, 3H). |
| | Starting materials: Int-1 and Int-50 | |
| | Reaction conditions: A | |
| 23 | 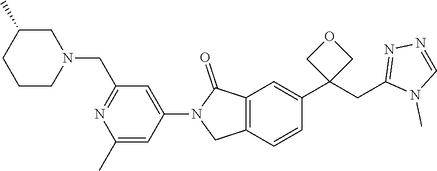 | LCMS: 531.2 [M + HCO₂H]⁻ . ¹H NMR (400 MHz, CD₃OD): δ 8.20 (s, 1H), 7.95 (d, 1H), 7.80 (d, 1H), 7.63 (dd, 1H), 7.50 (d, 1H), 7.27 (dd, 1H), 5.13 (d, 2H), 5.08 (d, 2H), 5.06 (s, 2H), 3.70 (s, 2H), 3.64 (s, 2H), 2.97 (s, 3H), 2.95-2.87 (m, 2H), 2.57 (s, 3H), 2.10-2.04 (m, 1H), 1.77-1.68 (m, 5H), 0.97-0.94 (m, 1H), 0.93 (d, 3H). |
| | Starting materials: Int-1 and Int-15 | |
| | Reaction conditions: B | |
| 24 | 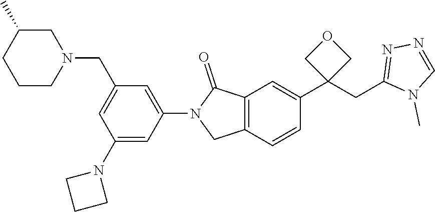 [SMILES: C[C@H]1CCCN(Cc2cc(N3CCC3)cc(N3Cc4ccc(C5(Cc6nncn6C)COC5)cc4C3=O)c2)C1] | LCMS: 527.30 [M + H]⁺. ¹H NMR (400 MHz, CD₃OD): δ 8.20 (s, 1H), 7.58 (dd, 1H), 7.43 (d, 1H), 7.20 (dd, 1H), 7.09 (s, 1H), 7.01 (t, 1H), 6.37 (t, 1H), 5.15 (d, 2H), 5.12 (d, 2H), 4.95 (s, 2H), 3.92 (t, 4H), 3.69 (s, 2H), 3.52 (s, 2H), 2.95-2.85 (m, 2H), 2.94 (s, 3H), 2.55-2.35 (m, 2H), 2.05-1.95 (m, 1H), 1.70-1.65 (m, 4H), 1.65-1.58 (m, 1H), 0.95-0.85 (m, 1H), 0.88 (d, 3H). |
| | Starting materials: Int-1 and Int-7 | |
| | Reaction conditions: A | |
| 25 | 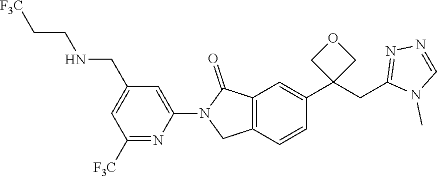 [SMILES: Cn1cnnc1CC1(c2ccc3c(c2)C(=O)N(c2cc(CNCCC(F)(F)F)cc(C(F)(F)F)n2)C3)COC1] | LCMS: 555.1 [M + H]⁺. ¹H NMR (400 MHz, CD₃OD): δ 8.78 (brs, 1H), 8.16 (s, 1H), 7.60 (d, 2H), 7.47 (d, 1H), 7.22 (dd, 1H), 5.12 (s, 2H), 5.09 (d, 2H), 5.05 (d, 2H), 3.93 (s, 2H), 3.66 (s, 2H), 2.92 (s, 3H), 2.85 (t, 2H), 2.46-2.39 (m, 2H). |
| | Starting materials: Int-1 and Int-51 | |
| | Reaction conditions: B | |
| 26 | 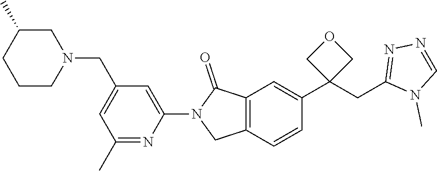 | LCMS: 531.1 [M + HCO₂H]⁻ . ¹H NMR (400 MHz, DMSO-d6): δ 8.32 (s, 1H), 8.20 (s, 1H), 7.62 (dd, 1H), 7.47 (d, 1H), 7.23 (dd, 1H), 7.07 (s, 1H), 5.14 (s, 2H), 5.13 (d, 2H), 5.09 (d, 2H), 3.70 (s, 2H), 3.55 (s, 2H), 2.94 (s, 2H), 2.90-2.80 (m, 2H), 2.52 (s, 3H), 2.10-1.95 (m, 2H), 1.80-1.60 (m, 6H), 0.95-0.90 (m, 1H), 0.89 (d, 3H). |
| | Starting materials: Int-1 and Int-17 | |
| | Reaction conditions: B | |
| 27 | 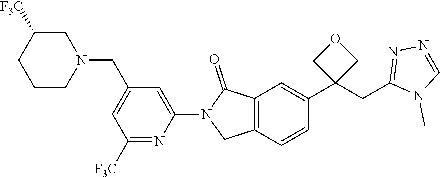 [SMILES: Cn1cnnc1CC1(c2ccc3c(c2)C(=O)N(c2cc(CN4CCC[C@H](C(F)(F)F)C4)cc(C(F)(F)F)n2)C3)COC1] | LCMS: 595.2 [M + H]⁺. ¹H NMR (400 MHz, CD₃OD): δ 8.80 (s, 1H), 8.16 (s, 1H), 7.59 (d, 1H), 7.55 (s, 1H), 7.46 (s, 1H), 7.23 (dd, 1H), 5.12 (s, 2H), 5.09 (d, 2H), 5.02 (d, 2H), 3.71 (d, 2H), 3.66 (s, 2H), 3.02 (d, 1H), 2.91 (s, 3H), 2.85 (d, 1H), 2.51-2.44 (m, 1H), 2.15- 1.94 (m, 3H), 1.82-1.62 (m, 1H), 1.39- 1.27 (m, 1H). |
| | Starting materials: Int-1 and Int-52 | |
| | Reaction conditions: B | |
| 28 | 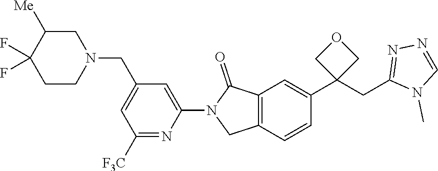 [SMILES: CC1CN(Cc2cc(N3Cc4ccc(C5(Cc6nncn6C)COC5)cc4C3=O)nc(C(F)(F)F)c2)CCC1(F)F] | LCMS: 577.20 [M + H]⁺. ¹H NMR (400 MHz, DMSO-d6): δ 8.78 (s, 1H), 8.18 (s, 1H), 7.61 (d, 1H), 7.57 (s, 1H), 7.51 (s, 1H), 7.34 (d, 1H), 5.03 (s, 2H), 4.94 (d, 2H), 4.87 (d, 2H), 3.76-3.66 (m, 2H), 3.54 (s, 2H), 2.99 (s, 3H), 2.79-2.68 (m, 2H), 2.40-2.28 (m, 1H), 2.13-1.88 (m, 4H), 0.92 (d, 3H). |
| | Starting materials: Int-1 and Int-25 | |
| | Reaction conditions: B | |
| 29 | 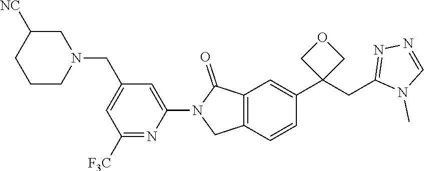 | LCMS: 596.2 [M + HCO₂H]⁻ . ¹H NMR (DMSO-d6, 400 MHz): δ 8.74 (s, 1H), 8.21 (s, 1H), 7.67 (s, 1H), 7.66 (d, 1H), 7.53 (d, 1H), 7.38 (dd, 1H), 5.08 (s, 2H), 4.98 (d, 2H), 4.91 (d, 2H), 3.74 (q, 2H), 3.58 (s, 2H), 3.10-3.04 (m, 1H), 3.03 (s, 3H), 2.61-2.51 (m, 1H), 2.43-2.35 (m, 1H), 2.03-1.99 (m, 1H), 1.76-1.68 (m, 2H), 1.62-1.53 (m, 1H), 1.49-1.41 (m, 1H). |
| | Starting materials: Int-1 and Int-53 | |
| | Reaction conditions: B | |
| 30 | 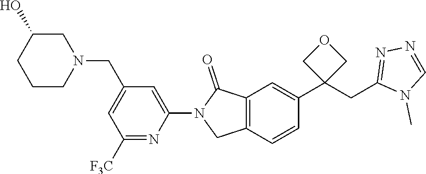 [SMILES: Cn1cnnc1CC1(c2ccc3c(c2)C(=O)N(c2cc(CN4CCC[C@H](O)C4)cc(C(F)(F)F)n2)C3)COC1] | LCMS: 543.2[M + H]⁺. ¹H NMR (DMSO- d6, 400 MHz): δ 8.78 (s, 1H), 8.22 (s, 1H), 7.66 (d, 1H), 7.59 (s, 1H), 7.53 (d, 1H), 7.38 (dd, 1H), 5.07 (s, 2H), 4.98 (d, 2H), 4.91 (d, 2H), 4.64 (d, 2H), 3.71 (s, 1H), 3.63 (s, 1H), 3.58 (s, 2H), 3.55-3.49 (m, 1H), 3.03 (s, 3H), 2.85-2.78 (m, 1H), 2.71-2.64 (m, 1H), 2.01-1.94 (m, 1H), 1.88-1.79 (m, 2H), 1.69-1.61 (m, 1H), 1.54-1.41 (m, 1H), 1.14-1.02 (m, 1H). |
| | Starting materials: Int-1 and Int-54 | |
| | Reaction conditions: B | |
| 31 | 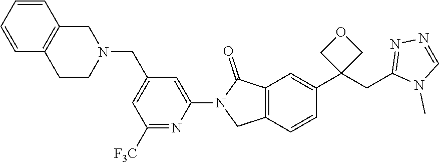 | LCMS: 619.1 [M + HCO₂H]⁻. ¹H NMR (DMSO-d6, 400 MHz): δ 8.87 (s, 1H), 8.21 (s, 1H), 7.66 (d, 2H), 7.51 (d, 1H), 7.38 (dd, 1H), 7.18-7.09 (m, 3H), 7.06- 7.01 (m, 1H), 5.08 (s, 2H), 4.98 (d, 2H), 4.90 (d, 2H), 3.88 (s, 2H), 3.66 (s, 2H), 3.57 (s, 2H), 3.02 (s, 3H), 2.91-2.86 (m, 2H), 2.78-2.73 (m, 2H). |
| | Starting materials: Int-1 and Int-55 | |
| | Reaction conditions: B | |
| 32 | 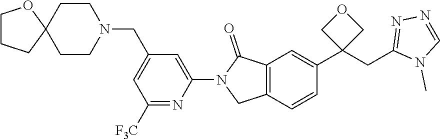 | LCMS: 627.2 [M + HCO₂H]⁻. ¹H NMR (CD₃OD, 400 MHz): δ 8.84 (s, 1H), 8.20 (s, 1H), 7.64 (d, 1H), 7.62 (d, 1H) 7.50 (d, 1H), 7.28 (dd, 1H), 5.16 (s, 2H), 5.13 (d, 2H), 5.09 (d, 2H), 3.87 (t, 2H), 3.71 (s, 4H), 3.58 (s, 2H), 2.97 (s, 3H), 2.58-2.49 (m, 4H), 1.82 (t, 2H), 1.72-1.67 (m, 4H). |
| | Starting materials: Int-1 and Int-56 | |
| | Reaction conditions: B | |
| 33 | 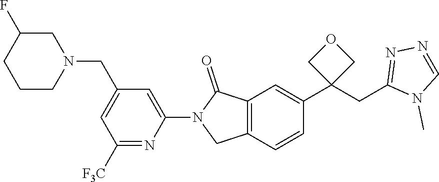 [SMILES: Cn1cnnc1CC1(c2ccc3c(c2)C(=O)N(c2cc(CN4CCCC(F)C4)cc(C(F)(F)F)n2)C3)COC1] | LCMS: 545.2 [M + H]⁺. ¹H NMR (CD₃OD, 400 MHz): δ 8.82 (s, 1H), 8.20 (s, 1H), 7.65-7.60 (m, 2H), 7.50 (d, 1H), 7.27 (dd, 1H), 5.16 (s, 2H), 5.13 (d, 2H), 5.09 (d, 2H), 4.80-4.55 (m, 1H), 3.74 (s, 2H), 3.70 (s, 2H), 2.96 (s, 3H), 2.84-2.80 (m, 1H), 2.57-2.52 (m, 2H), 2.45-2.43 (m, 1H), 1.97-1.85 (m, 2H), 1.70-1.54 (m, 2H). |
| | Starting materials: Int-1 and Int-57 | |
| | Reaction conditions: B | |
| 34 | 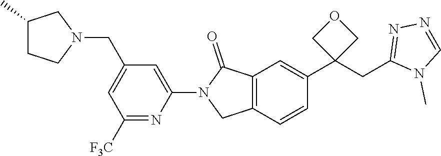 | LCMS: 571.1 [M + HCO₂H]⁻. ¹H NMR (DMSO-d6, 400 MHz): δ 8.79 (s, 1H), 8.22 (s, 1H), 7.66 (d, 1H), 7.58 (d, 1H), 7.53 (d, 1H), 7.38 (dd, 1H), 5.07 (s, 2H), 4.98 (d, 2H), 4.91 (d, 2H), 3.77 (q, 2H), 3.58 (s, 2H), 3.03 (s, 3H), 2.78-2.70 (m, 1H), 2.65-2.52 (m, 2H), 2.28-2.19 (m, 1H), 2.15-2.05 (m, 1H), 2.03-1.91 (m, 1H), 1.38-1.25 (m, 1H), 1.01 (d, 3H). |
| | Starting materials: Int-1 and Int-58 | |
| | Reaction conditions: B | |
| 35 | 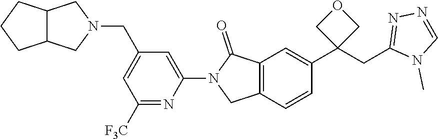 [SMILES: Cn1cnnc1CC1(c2ccc3c(c2)C(=O)N(c2cc(CN4CC5CCCC5C4)cc(C(F)(F)F)n2)C3)COC1] | LCMS: 553.2 [M + H]⁺. ¹H NMR (DMSO- d6, 400 MHz): δ 8.81 (s, 1H), 8.22 (s, 1H), 7.65 (d, 1H), 7.57 (s, 1H), 7.55 (d, 1H), 7.36 (dd, 1H), 5.07 (s, 2H), 4.98 (d, 2H), 4.91 (d, 2H), 3.71 (s, 2H), 3.58 (s, 2H), 3.03 (s, 3H), 2.66-2.55 (m, 2H), 2.36-2.28 (m, 2H), 1.75-1.62 (m, 4H), 1.50-1.35 (m, 4H). |
| | Starting materials: Int-1 and Int-59 | |
| | Reaction conditions: B | |
| 36 | 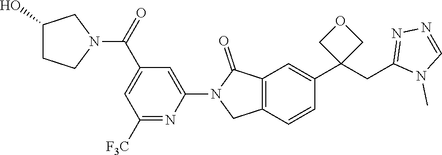 [SMILES: Cn1cnnc1CC1(c2ccc3c(c2)C(=O)N(c2cc(C(=O)N4CC[C@H](O)C4)cc(C(F)(F)F)n2)C3)COC1] | LCMS: 543.1 [M + H]⁺. ¹H NMR (400 MHz, DMSO-d6): δ 8.83 (d, 1H), 8.21 (s, 1H), 7.74 (s, 1H), 7.67 (d, 1H), 7.55 (s, 1H), 7.39 (dd, 1H), 5.09 (s, 2H), 5.08 (brs, 1H), 4.97 (d, 2H), 4.90 (d, 2H), 4.40-4.35 (brm, 1H), 4.30-4.24 (brm, 1H), 3.65-3.53 (m, 3H), 3.48-3.40 (m, 1H), 3.20-3.18 (brm, 1H), 3.03 (s, 3H), 2.00-1.87 (m, 2H). |
| | Starting materials: Int-1 and Int-26 | |
| | Reaction conditions: B | |
| 37 | 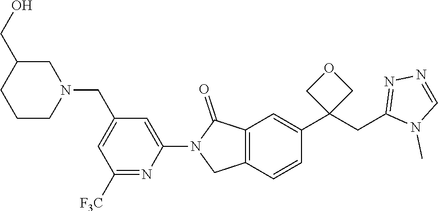 [SMILES: Cn1cnnc1CC1(c2ccc3c(c2)C(=O)N(c2cc(CN4CCCC(CO)C4)cc(C(F)(F)F)n2)C3)COC1] | LCMS: 557.2 [M + H]⁺. ¹H NMR (DMSO-d6, 400 MHz): δ 8.80 (s, 1H), 8.22 (s, 1H), 7.66 (d, 1H), 7.59 (s, 1H), 7.53 (d, 1H), 7.38 (dd, 1H), 5.07 (s, 2H), 4.98 (d, 2H), 4.91 (d, 2H), 4.41 (t, 1H), 3.64 (s, 2H), 3.58 (s, 2H), 3.32-3.28 (m, 1H), 3.25-3.17 (m, 1H), 3.02 (s, 3H), 2.89- 2.80 (m, 1H), 2.75-2.70 (m, 1H), 2.02 (t, 1H), 1.79 (t, 1H), 1.72-1.60 (m, 3H), 1.59-1.42 (m, 1H), 1.10-0.89 (m, 1H). |
| | Starting materials: Int-1 and Int-60 | |
| | Reaction conditions: B | |
| 38 | 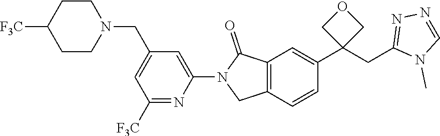 [SMILES: Cn1cnnc1CC1(c2ccc3c(c2)C(=O)N(c2cc(CN4CCC(C(F)(F)F)CC4)cc(C(F)(F)F)n2)C3)COC1] | LCMS: 595.2 [M + H]⁺. ¹H NMR (DMSO- d6, 400 MHz): δ 8.81 (s, 1H), 8.22 (s, 1H), 7.66 (d, 1H), 7.59 (s, 1H), 7.55 (d, 1H), 7.38 (dd, 1H), 5.07 (s, 2H), 4.98 (d, 2H), 4.91 (d, 2H), 3.70 (s, 2H), 3.58 (s, 2H), 3.03 (s, 3H), 2.95-2.89 (m, 2H), 2.38-2.28 (m, 1H), 2.15-2.05 (m, 2H), 1.85-1.78 (m, 2H), 1.60-1.49 (m, 2H). |
| | Starting materials: Int-1 and Int-61 | |
| | Reaction conditions: B | |
| 39 | 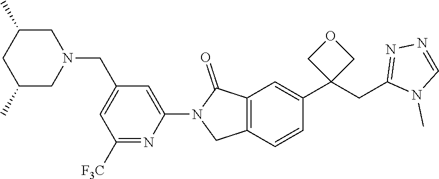 [SMILES: C[C@@H]1C[C@H](C)CN(Cc2cc(N3Cc4ccc(C5(Cc6nncn6C)COC5)cc4C3=O)nc(C(F)(F)F)c2)C1] | LCMS: 555.5 [M + H]⁺. ¹H NMR (DMSO- d6, 400 MHz): δ 8.79 (s, 1H), 8.22 (s, 1H), 7.66 (d, 1H), 7.57 (s, 1H), 7.53 (d, 1H), 7.38 (dd, 1H), 5.07 (s, 2H), 4.98 (d, 2H), 4.91 (d, 2H), 3.64 (s, 2H), 3.58 (s, 2H), 3.03 (s, 3H), 2.80-2.74 (m, 2H), 1.74-1.63 (m, 3H), 1.60-1.52 (m, 2H), 0.81 (d, 6H), 0.54 (q, 1H). |
| | Starting materials: Int-1 and Int-62 | |
| | Reaction conditions: B | |
| 40 | 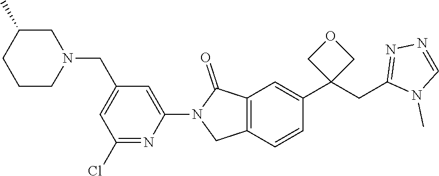 [SMILES: C[C@H]1CCCN(Cc2cc(Cl)nc(N3Cc4ccc(C5(Cc6nncn6C)COC5)cc4C3=O)c2)C1] | LCMS: 507.2 M + H]⁺. ¹H-NMR (400 MHz, DMSO-d6): δ 8.49 (s, 1H), 8.21 (s, 1 H), 7.62 (d, 1H), 7.51 (d, 1H), 7.36 (dd, 1H), 7.20 (s, 1H), 5.02 (s, 2H), 4.98 (d, 2H), 4.90 (d, 2H), 3.57 (s, 2H), 3.53 (s, 2H), 3.02 (s, 3H), 2.78-2.70 (m, 2H), 1.98-1.90 (m, 1H), 1.71-1.57 (m, 5H), 0.91-0.82 (m, 1H), 0.84 (d, 3H). |
| | Starting materials: Int-1 and Int-16 | |
| | Reaction conditions: B | |
| 41 | 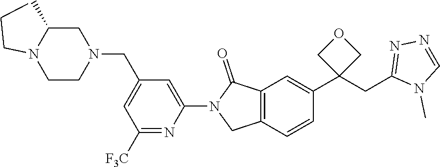 | LCMS: 612.2 [M + HCO₂H]⁻. ¹H-NMR (400 MHz, DMSO-d6): δ 8.80 (s, 1H), 8.22 (s, 1 H), 7.66 (d, 1H), 7.58 (s, 1H), 7.53 (d, 1H), 7.38 (dd, 1H), 5.07 (s, 2H), 4.98 (d, 2H), 4.91 (d, 2H), 3.82-3.65 (m, 2H), 3.58 (s, 2H), 3.03 (s, 3H), 2.88-2.85 (m, 3H), 2.80-2.72 (m, 1H), 2.28-2.15 (m, 2H), 2.12-2.01 (m, 2H), 1.92 (t, 1H), 1.75-1.60 (m, 3H), 1.31-1.18 (m, 1H). |
| | Starting materials: Int-1 and Int-63 | |
| | Reaction conditions: B | |
| 42 | 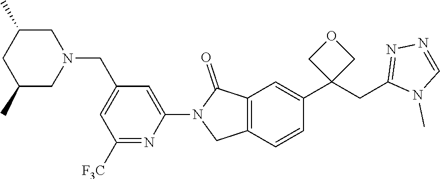 [SMILES: C[C@H]1C[C@H](C)CN(Cc2cc(N3Cc4ccc(C5(Cc6nncn6C)COC5)cc4C3=O)nc(C(F)(F)F)c2)C1] | LCMS: 555.2 [M + H]⁺. ¹H NMR (DMSO- d6, 400 MHz): δ 8.84 (s, 1H), 8.23 (bs, 1H), 7.66 (d, 1H), 7.57 (d, 2H), 7.37 (dd, 1H), 5.07 (s, 2H), 4.97 (d, 2H), 4.91 (d, 2H), 3.72-3.65 (m, 1H), 3.60 (s, 2H), 3.52 (d, 1H), 3.04 (s, 3H), 2.46-2.38 (m, 2H), 2.13-2.03 (m, 2H), 1.96-1.88 (m, 2H), 1.35-1.28 (m, 2H), 0.98 (d, 6H). |
| | Starting materials: Int-1 and Int-64 | |
| | Reaction conditions: B | |
| 43 | 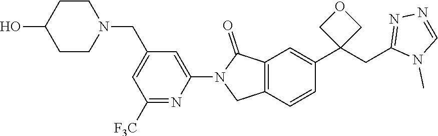 [SMILES: Cn1cnnc1CC1(c2ccc3c(c2)C(=O)N(c2cc(CN4CCC(O)CC4)cc(C(F)(F)F)n2)C3)COC1] | LCMS: 543.2 [M + H]⁺. ¹H NMR (400 MHz, DMSO-d6): δ 8.76 (s, 1H), 8.18 (s, 1H), 7.62 (d, 1H), 7.54 (s, 1H), 7.49 (s, 1H), 7.34 (dd, 1H), 5.03 (s, 2H), 4.94 (d, 2H), 4.87 (s, 2H), 4.55 (d, 1H), 3.60 (s, 2H), 3.54 (s, 2H), 3.48-3.44 (m, 1H), 2.99 (s, 3H), 2.68-2.62 (m, 2H), 2.15-2.08 (m, 2H), 1.72-1.68 (m, 2H), 1.46-1.38 (m, 2H). |
| | Starting materials: Int-1 and Int-65 | |
| | Reaction conditions: B | |
| 44 | 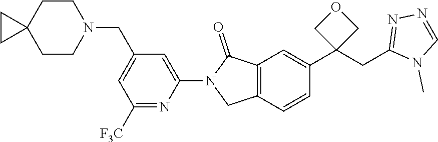 [SMILES: Cn1cnnc1CC1(c2ccc3c(c2)C(=O)N(c2cc(CN4CCC5(CC4)CC5)cc(C(F)(F)F)n2)C3)COC1] | LCMS: 553.3 [M + H]⁺. ¹H NMR (400 MHz, DMSO-d6): δ 8.78 (s, 1H), 8.18 (s, 1H), 7.62 (d, 1H), 7.56 (s, 1H), 7.49 (s, 1H), 7.34 (dd, 1H), 5.03 (s, 2H), 4.94 (d, 2H), 4.88 (d, 2H), 3.66 (s, 2H), 3.54 (s, 2H), 2.99 (s, 3H), 2.48-2.43 (m, 4H), 1.35-1.30 (m, 4H), 0.23 (s, 4H). |
| | Starting materials: Int-1 and Int-66 | |
| | Reaction conditions: B | |
| 45 | 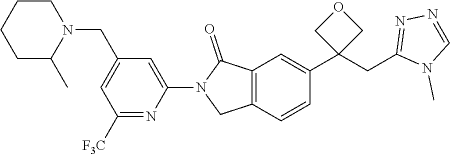 | LCMS: 585.2 [M + HCO₂H]⁻. ¹H NMR (CD3OD, 400 MHz): δ 8.83 (s, 1H), 8.20 (s, 1H), 7.64 (dd, 1H), 7.60 (s, 1H), 7.49 (s, 1H), 7.27 (dd, 1H), 5.16 (s, 2H), 5.15 (d, 2H), 5.10 (d, 2H), 4.15 (d, 1H), 3.70 (s, 2H), 3.45 (d, 1H), 2.96 (s, 3H), 2.81- 2.73 (m, 1H), 2.49-2.40 (m, 1H), 2.19- 2.10 (m, 1H), 1.74 (d, 2H), 1.63-1.54 (m, 2H), 1.35-1.30 (m, 2H), 1.20 (d, 3H). |
| | Starting materials: Int-1 and Int-67 | |
| | Reaction conditions: B | |
| 46 | 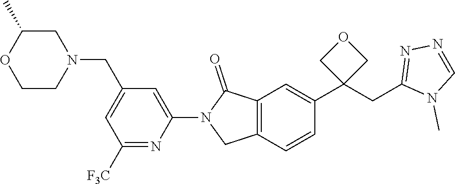 [SMILES: C[C@@H]1CN(Cc2cc(N3Cc4ccc(C5(Cc6nncn6C)COC5)cc4C3=O)nc(C(F)(F)F)c2)CCO1] | LCMS: 543.2 [M + H]⁺. ¹H NMR (400 MHz, DMSO-d6): δ 8.79 (s, 1H), 8.21 (s, 1H), 7.65 (d, 1H), 7.59 (s, 1H), 7.52 (brs, 1H), 7.37 (dd, 1H), 5.06 (s, 2H), 4.97 (d, 2H), 4.90 (d, 2H), 3.76 (d, 1H), 3.66 (s, 2H), 3.60-3.50 (m, 2H), 3.57 (s, 2H), 3.02 (s, 3H), 2.72 (d, 1H), 2.64 (d, 1H), 2.16-2.21 (m, 1H), 1.83 (t, 1H), 1.04 (d, 3H). |
| | Starting materials: Int-1 and Int-68 | |
| | Reaction conditions: B | |
| 47 | 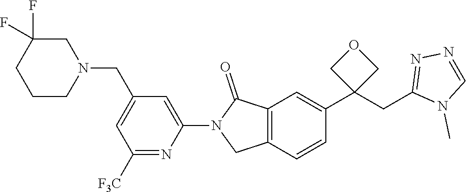 [SMILES: Cn1cnnc1CC1(c2ccc3c(c2)C(=O)N(c2cc(CN4CCCC(F)(F)C4)cc(C(F)(F)F)n2)C3)COC1] | LCMS: 563.2 [M + H]⁺. ¹H NMR (DMSO- d6, 400 MHz): δ 8.78 (s, 1H), 8.21 (s, 1H), 7.65 (d, 1H), 7.59 (s, 1H), 7.53 (s, 1H), 7.37 (dd, 1H), 5.06 (s, 2H), 4.97 (d, 2H), 4.90 (d, 2H), 3.79 (s, 2H), 3.57 (s, 2H), 3.02 (s, 3H), 2.78-2.69 (m, 2H), 2.48-2.43 (m, 2H), 1.95-1.86 (m, 2H), 1.73-1.66 (m, 2H). |
| | Starting materials: Int-1 and Int-69 | |
| | Reaction conditions: B | |
| 48 | 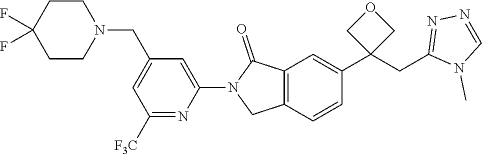 | LCMS: 607.1 [M + H]⁺. ¹H NMR (CD₃OD, 400 MHz): δ 8.86 (s, 1H), 7.64 (d, 1H), 7.60 (s, 1H), 7.50 (d, 1H), 7.28 (dd, 1H), 5.16 (s, 2H), 5.15 (d, 2H), 5.10 (d, 2H), 3.77 (s, 2H), 3.70 (s, 2H), 2.97 (s, 3H), 2.70-2.61 (m, 4H), 2.12-2.00 (m, 4H). |
| | Staring materials: Int-1 and Int-70 | |
| | Reaction conditions: B | |
| 49 | 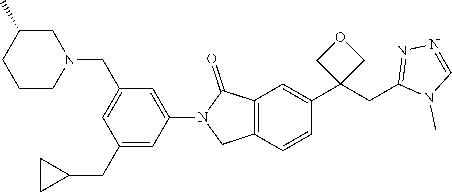 [SMILES: C[C@H]1CCCN(Cc2cc(CC3CC3)cc(N3Cc4ccc(C5(Cc6nncn6C)COC5)cc4C3=O)c2)C1] | LCMS: 526.20 [M +H]⁺. ¹H NMR (400 MHz, DMSO-d6): δ 8.21 (s, 1H), 7.67 (s, 2H), 7.56 (d, 1H), 7.45 (d, 1H), 7.29 (dd, 1H), 7.00 (s, 1H), 5.05-4.95 (m, 4H), 4.90 (d, 2H), 3.57 (s, 2H), 3.43 (s, 2H), 3.00 (s, 3H), 2.80-2.70 (m, 2H), 2.53 (d, 2H), 1.92-1.82 (m, 1H), 1.70- 1.55 (m, 4H), 1.52-1.42 (m, 1H), 1.05- 0.95 (m, 1H), 0.92-0.83 (m, 1H), 0.82 (d, 3H), 0.55-0.45 (m, 2H), 0.25-0.18 (m, 2H). |
| | Starting materials: Int-1 and Int-11 | |
| | Reaction conditions: A | |
| 50 | 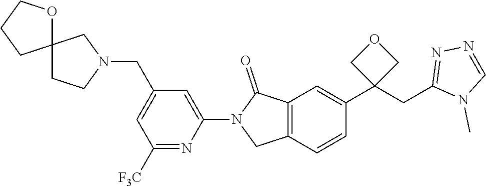 [SMILES: Cn1cnnc1CC1(c2ccc3c(c2)C(=O)N(c2cc(CN4CCC5(CCCO5)C4)cc(C(F)(F)F)n2)C3)COC1] | LCMS: 569.2 [M + H]⁺. ¹H NMR (400 MHz, DMSO-d6): δ 8.74 (s, 1H), 8.18 (s, 1H), 7.62 (d, 1H), 7.55 (s, 1H), 7.49 (d, 1H), 7.34 (dd, 1H), 5.03 (s, 2H), 4.94 (d, 2H), 4.87 (d, 2H), 3.78-3.58 (m, 4H), 3.54 (s, 2H), 2.99 (s, 3H), 2.65-2.50 (m, 4H), 1.89-1.74 (m, 6H). |
| | Starting materials: Int-1 and Int-56 | |
| | Reaction conditions: B | |
| 51 | 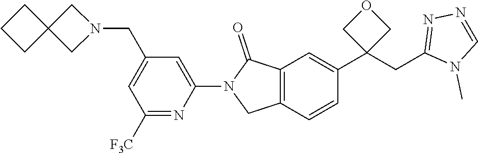 [SMILES: Cn1cnnc1CC1(c2ccc3c(c2)C(=O)N(c2cc(CN4CC5(CCC5)C4)cc(C(F)(F)F)n2)C3)COC1] | LCMS: 539.2 [M + H]⁺. ¹H NMR (400 MHz, DMSO-d6): δ 8.68 (s, 1H), 8.18 (s, 1H), 7.61 (d, 1H), 7.49 (d, 2H), 7.34 (dd, 1H), 5.02 (s, 2H), 4.94 (d, 2H), 4.87 (d, 2H), 3.66 (s, 2H), 3.54 (s, 2H), 3.17 (s, 2H), 2.99 (s, 2H), 2.04 (t, 4H), 1.77-1.67 (m, 2H). |
| | Starting materials: Int-1 and Int-72 | |
| | Reaction conditions: B | |
| 52 | 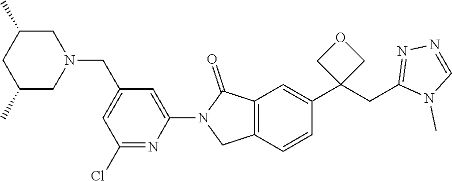 [SMILES: C[C@@H]1C[C@H](C)CN(Cc2cc(Cl)nc(N3Cc4ccc(C5(Cc6nncn6C)COC5)cc4C3=O)c2)C1] | LCMS: 521.2 [M + H]⁺. ¹H NMR (400 MHz, DMSO-d6): δ 8.49 (s, 1H), 8.21 (s, 1 H), 7.62 (d, 1H), 7.51 (d, 1H), 7.36 (dd, 1H), 7.20 (s, 1H), 5.03 (s, 2H), 4.98 (d, 2H), 4.91 (d, 2H), 3.57 (s, 2H), 3.54 (s, 2H), 3.02 (s, 3H), 2.77-2.74 (m, 2H), 1.75-1.60 (m, 2H), 1.5 (t, 2H), 0.90-0.85 (m, 1H), 0.81 (d, 6H), 0.52 (q, 1H). |
| | Starting materials: Int-1 and Int-73 | |
| | Reaction conditions: B | |
| 53 | 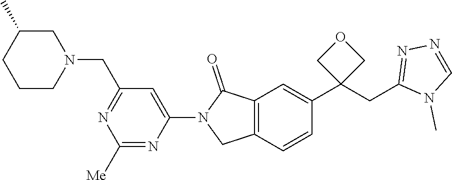 [SMILES: Cc1nc(CN2CCC[C@H](C)C2)cc(N2Cc3ccc(C4(Cc5nncn5C)COC4)cc3C2=O)n1] | LCMS: 488.20 [M + H]⁺. ¹H NMR (400 MHz, CD₃OD): δ 8.50 (s, 1H), 8.16 (s, 1H), 7.59 (d, 1H), 7.46 (s, 1H), 7.25 (dd, 1H), 5.09 (d, 4H), 5.04 (d, 2H), 3.66 (s, 2H), 3.57 (s, 2H), 2.93 (s, 3H), 2.90- 2.80 (m, 2H), 2.61 (s, 3H), 2.10-2.00 (m, 1H), 1.78-1.68 (m, 4H), 1.68-1.62 (m, 1H), 0.98-0.88 (m, 1H), 0.87 (d, 3H). |
| | Starting materials: Int-1 and Int-13 | |
| | Reaction conditions: B | |
| 54 | 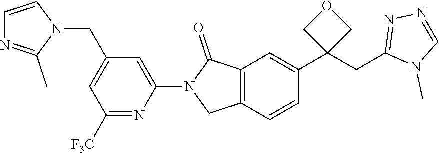 [SMILES: Cc1nccn1Cc1cc(N2Cc3ccc(C4(Cc5nncn5C)COC4)cc3C2=O)nc(C(F)(F)F)c1] | LCMS: 524.2 [M + H]⁺. ¹H NMR (DMSO- d6, 400 MHz): δ 8.46 (s, 1H), 8.21 (s, 1H), 7.65 (d, 1H), 7.51 (d, 1H), 7.39 (s, 1H), 7.38 (d, 1H), 7.20 (d, 1H), 6.85 (d, 1H), 5.42 (s, 2H), 5.05 (s, 2H), 4.97 (d, 2H), 4.90 (d, 2H), 3.57 (s, 2H), 3.02 (s, 3H), 2.24 (s, 3H). |
| | Starting materials: Int-1 and Int-28 | |
| | Reaction conditions: B | |
| 55 | 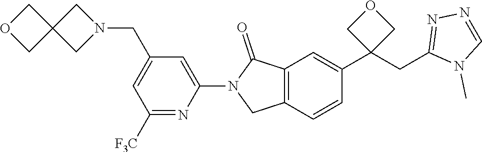 [SMILES: Cn1cnnc1CC1(c2ccc3c(c2)C(=O)N(c2cc(CN4CC5(COC5)C4)cc(C(F)(F)F)n2)C3)COC1] | LCMS: 541.1 [M + H]⁺. δH NMR (400 MHz, DMSO-d6): δ 8.67 (brs, 1H), 8.18 (s, 1H), 7.62 (d, 1H), 7.53-7.42 (m, 2H), 7.35 (d, 1H), 5.02 (s, 2H), 4.94 (d, 2H), 4.87 (d, 2H), 4.60 (s, 4H), 3.68 (brs, 2H), 3.54 (s, 2H), 3.38 (brs, 4H), 2.99 (s, 3H). |
| | Starting materials: Int-1 and Int-74 | |
| | Reaction conditions: B | |
| 56 | 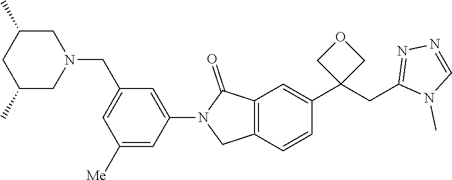 [SMILES: Cc1cc(CN2C[C@H](C)C[C@H](C)C2)cc(N2Cc3ccc(C4(Cc5nncn5C)COC4)cc3C2=O)c1] | LCMS: 500.3 [M + H]⁺. ¹H NMR (CD₃OD, 400 MHz): δ 8.17 (s, 1H), 7.63 (s, 1H), 7.61 (s, 1H), 7.56 (d, 1H), 7.42 (d, 1H), 7.17 (dd, 1H), 7.03 (s, 1H), 5.09 (d, 2H), 5.04 (d, 2H), 4.95 (s, 2H), 3.67 (s, 2H), 3.56 (s, 2H), 2.91 (s, 3H), 2.90-2.86 (m, 2H), 2.38 (s, 3H), 1.77-1.67 (m, 3H), 1.64-1.54 (m, 2H), 0.85 (d, 6H), 0.6-0.50 (m, 1H). |
| | Starting materials: Int-1 and Int-75 | |
| | Reaction conditions: A | |
| 57 | 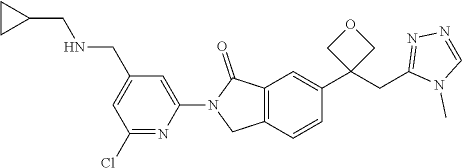 [SMILES: Cn1cnnc1CC1(c2ccc3c(c2)C(=O)N(c2cc(CNCC4CC4)cc(Cl)n2)C3)COC1] | LCMS: 479.1 [M + H]⁺. ¹H NMR (400 MHz, DMSO-d6): δ 8.49 (s, 1H), 8.21 (s, 1 H), 7.62 (d, 1H), 7.50 (d, 1H), 7.37 (dd, 1H), 7.27 (d, 1H), 5.03 (s, 2H), 4.98 (d, 2H), 4.91 (d, 2H), 3.83 (s, 2H), 3.57 (s, 2H), 3.03 (s, 3H), 2.40 (d, 2H), 0.98-0.70 (m, 1H), 0.47-0.38 (m, 2H), 0.16-0.10 (m, 2H). |
| | Starting materials: Int-1 and Int-76 | |
| | Reaction conditions: B | |
| 58 | 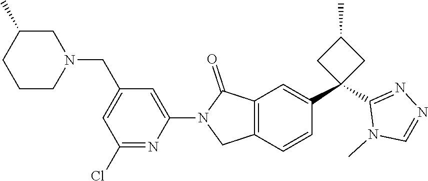 [SMILES: C[C@H]1CCCN(Cc2cc(Cl)nc(N3Cc4ccc([C@]5(c6nncn6C)C[C@@H](C)C5)cc4C3=O)c2)C1] | LCMS: 507.2 [M + 2 + H]⁺. ¹H NMR (400 MHz, DMSO-d6): δ 8.51 (s, 1H), 8.31 (s, 1H), 7.75-7.65 (m, 3H), 7.20 (s, 1H), 5.05 (s, 2H), 3.54 (s, 2H), 3.18 (s, 3H), 2.91-2.84 (m, 2H), 2.78-2.68 (m, 2H), 2.62-2.55 (m, 2H), 2.05-1.91 (m, 1H), 1.70-1.60 (m, 4H), 1.55-1.45 (m, 1H), 1.10 (d, 3H), 0.92-0.80 (m, 1H), 0.82 (d, 3H). |
| | Starting materials: Int-1 and Int-18 | |
| | Reaction conditions: B | |
| 59 | 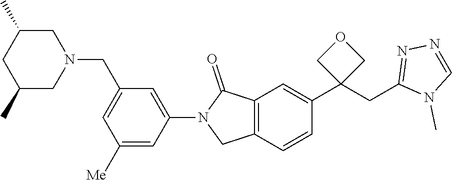 [SMILES: Cc1cc(CN2C[C@@H](C)C[C@H](C)C2)cc(N2Cc3ccc(C4(Cc5nncn5C)COC4)cc3C2=O)c1] | LCMS: 500.2 [M + H]⁺. ¹H NMR (DMSO, 400 MHz): δ 8.21 (s, 1H), 7.69 (s, 1H), 7.57-7.54 (m, 2H), 7.47 (d, 1H), 7.28 (dd, 1H), 6.93 (s, 1H), 4.98 (d, 2H), 4.94 (s, 2H), 4.91 (d, 2H), 3.57 (s, 2H), 3.44 (d, 1H), 3.33 (d, 1H), 3.01 (s, 3H), 2.40-2.35 (m, 2H), 2.34 (s, 3H), 2.03-2.01 (m, 2H), 1.90-1.80 (m, 2H), 1.28-1.21 (m, 2H), 0.94 (d, 6H). |
| | Starting materials: Int-1 and Int-77 | |
| | Reaction conditions: A | |
| 60 | 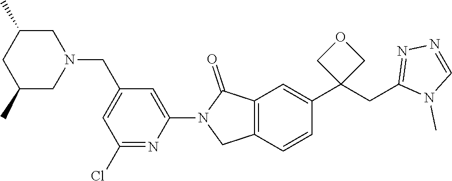 [SMILES: C[C@H]1C[C@H](C)CN(Cc2cc(Cl)nc(N3Cc4ccc(C5(Cc6nncn6C)COC5)cc4C3=O)c2)C1] | LCMS: 522.2 [M + H]⁺. ¹H NMR (400 MHz, CD3OD): δ 8.55 (d, 1H), 8.20 (s, 1H), 7.62 (dd, 1H), 7.49 (d, 1H), 7.25 (dd, 1H), 7.20 (d, 1H), 5.13 (d, 2H), 5.10 (s, 2H), 5.08 (d, 2H), 3.70 (s, 2H), 3.57 (d, 1H), 3.45 (d, 1H), 2.96 (s, 3H), 2.47 (m, 2H), 2.19- 2.10 (m, 2H), 2.01-1.93 (m, 2H), 1.40- 1.31 (m, 2H), 1.03 (d, 6H). |
| | Starting materials: Int-1 and Int-78 | |
| | Reaction conditions: B | |
| 61 | 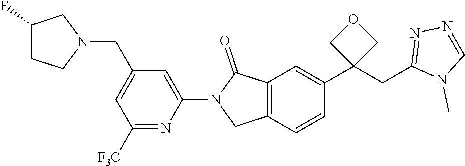 [SMILES: Cn1cnnc1CC1(c2ccc3c(c2)C(=O)N(c2cc(CN4CC[C@H](F)C4)cc(C(F)(F)F)n2)C3)COC1] | LCMS: 531.2 [M + H]⁺. ¹H NMR (400 MHz, DMSO-d6): δ 8.78 (s, 1H), 8.22 (s, 1H), 7.66 (d, 1H), 7.61 (s, 1H), 7.52 (d, 1H), 7.38 (dd, 1H), 5.35-5.10 (m, 1H), 5.08 (s, 2H), 4.98 (d, 2H), 4.91 (d, 2H), 3.83 (s, 2H), 3.58 (s, 2H), 3.02 (s, 3H), 2.92-2.82 (m, 2H), 2.78-2.72 (m, 1H), 2.48-2.42 (m, 1H), 2.28-2.12 (m, 1H), 2.05-1.95 (m, 1H). |
| | Starting materials: Int-1 and Int-79 | |
| | Reaction conditions: B | |
| 62 | 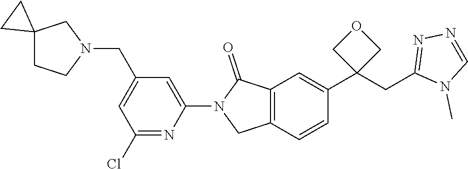 [SMILES: Cn1cnnc1CC1(c2ccc3c(c2)C(=O)N(c2cc(CN4CCC5(CC5)C4)cc(Cl)n2)C3)COC1] | LCMS: 505.2 [M + H]⁺. ¹H NMR (400 MHz, DMSO-d6): δ 8.49 (d, 1H), 8.22 (s, 1H), 7.62 (d, 1H), 7.50 (d, 1H), 7.37 (dd, 1H), 7.23 (d, 1H), 5.03 (s, 2H), 4.98 (d, 2H), 4.91 (d, 2H), 3.68 (s, 1H), 3.57 (s, 2H), 3.02 (s, 3H), 2.72 (t, 2H), 2.48 (s, 2H), 1.78 (t, 2H), 0.52 (m, 4H). |
| | Starting materials: Int-1 and Int-80 | |
| | Reaction conditions: B | |
| 63 | 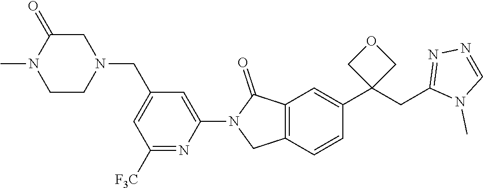 [SMILES: CN1CCN(Cc2cc(N3Cc4ccc(C5(Cc6nncn6C)COC5)cc4C3=O)nc(C(F)(F)F)c2)CC1=O] | LCMS: 556.2 [M + H]⁺. ¹H NMR (CD₃OD, 400 MHz): δ 8.87 (s, 1H), 8.20 (s, 1H), 7.64 (dd, 1H), 7.60 (d, 1H), 7.49 (d, 1H), 7.28 (dd, 1H), 5.16 (s, 2H), 5.13 (d, 2H), 5.09 (d, 2H), 3.80 (s, 2H), 3.70 (s, 2H), 3.49-3.42 (m, 2H), 3.21 (s, 2H), 3.00 (s, 3H), 2.96 (s, 3H), 2.83-2.81 (m, 2H). |
| | Starting materials: Int-1 and Int-31 | |
| | Reaction conditions: B | |
| 64 | 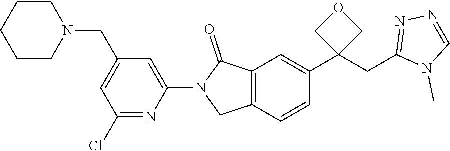 [SMILES: Cn1cnnc1CC1(c2ccc3c(c2)C(=O)N(c2cc(CN4CCCCC4)cc(Cl)n2)C3)COC1] | LCMS: 493.2 [M + H]⁺. ¹H NMR (400 MHz, CD₃OD): δ 8.49 (s, 1H), 8.21 (s, 1H), 7.62 (d, 1H), 7.50 (d, 2H), 7.37 (dd, 1H), 7.20 (d, 1H), 5.03 (s, 2H), 4.98 (d, 2H), 4.91 (d, 2H), 3.57 (s, 2H), 3.53 (s, 2H), 3.02 (s, 3H), 2.40-2.30 (m, 4H), 1.60-1.50 (m, 4H), 1.45-1.37 (m, 2H) |
| | Starting materials: Int-1 and Int-81 | |
| | Reaction conditions: B | |
| 65 | 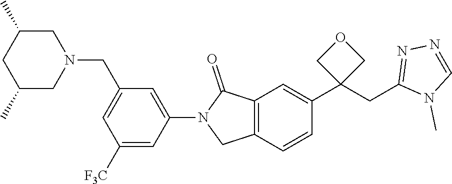 | LCMS: 598.2 [M + HCO₂H]⁻. ¹H NMR (400 MHz, DMSO-d6): δ 8.25 (s, 1H), 8.22 (s, 1H), 8.06 (s, 1H), 7.59 (d, 1H), 7.50 (d, 1H), 7.43 (s, 1H), 7.34 (dd, 1H), 5.07 (s, 2H), 4.97 (d, 2H), 4.92 (d, 2H), 3.58 (s, 2H), 3.57 (s, 2H), 3.02 (s, 3H), 2.80-2.75 (m, 2H), 1.72-1.62 (m, 3H), 1.55-1.45 (m, 2H), 0.81 (d, 6H), 0.55- 0.45 (m, 1H). |
| | Starting materials: Int-1 and Int-82 | |
| | Reaction conditions: A | |
| 66 | 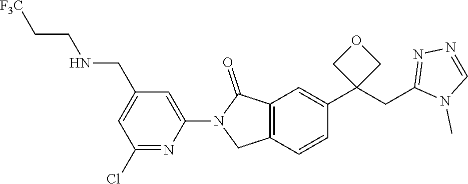 [SMILES: Cn1cnnc1CC1(c2ccc3c(c2)C(=O)N(c2cc(CNCCC(F)(F)F)cc(Cl)n2)C3)COC1] | LCMS: 521.1 [M + H]⁺. ¹H NMR (400 MHz, DMSO-d6) δ 8.47 (s, 1H), 8.21 (s, 1 H), 7.62 (d, 1H), 7.50 (d, 1H), 7.37 (dd, 1H), 7.27 (d, 1H), 5.03 (s, 2H), 4.98 (d, 2H), 4.91 (d, 2H), 3.81 (s, 2H), 3.57 (s, 2H), 3.01 (s, 3H), 2.72 (t, 2H), 1.98-1.89 (m, 2H). |
| | Starting materials: Int-1 and Int-83 | |
| | Reaction conditions: B | |
| 67 | 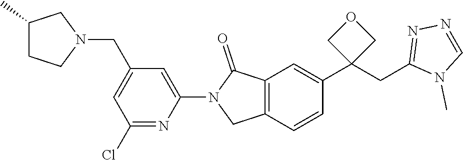 [SMILES: C[C@H]1CCN(Cc2cc(Cl)nc(N3Cc4ccc(C5(Cc6nncn6C)COC5)cc4C3=O)c2)C1] | LCMS: 493.0 [M + H]⁺. ¹H NMR (400 MHz, DMSO-d6): δ 8.49 (s, 1H), 8.21 (s, 1H), 7.62 (d, 1H), 7.50 (d, 1H), 7.37 (dd, 1H), 7.21 (brs, 1H), 5.03 (s, 2H), 4.98 (d, 2H), 4.91 (d, 2H), 3.71- 3.61 (m, 2H), 3.57 (s, 2H), 3.02 (s, 3H), 2.78-2.69 (m, 1H), 2.62-2.58 (m, 1H), 2.25-2.15 (m, 1H), 2.12-2.06 (m, 1H), 2.04-1.92 (m, 2H), 1.38-1.28 (m, 1H), 1.01 (d, 3H). |
| | Starting materials: Int-1 and Int-84 | |
| | Reaction conditions: B | |
| 68 | 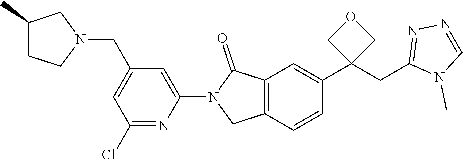 [SMILES: C[C@@H]1CCN(Cc2cc(Cl)nc(N3Cc4ccc(C5(Cc6nncn6C)COC5)cc4C3=O)c2)C1] | LCMS: 493.0 [M + H]⁺. ¹HNMR (400 MHz, DMSO-d6): δ 8.49 (d, 1H), 8.21 (s, 1H), 7.62 (d, 1H), 7.50 (d, 1H), 7.36 (dd, 1H), 7.21 (d, 1H), 5.03 (s, 2H), 4.98 (d, 2H), 4.91 (d, 2H), 3.71-3.61 (ABq, 2H), 3.57 (s, 2H), 3.02 (s, 3H), 2.75-2.70 (m, 1H), 2.62-2.58 (m, 1H), 2.28-2.18 (m, 1H), 2.11-2.06 (m, 1H), 2.03-1.93 (m, 2H), 1.36-1.28 (m, 1H), 1.01 (d, 3H). |
| | Starting materials: Int-1 and Int-85 | |
| | Reaction conditions: B | |
| 69 | 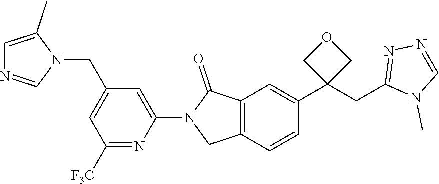 [SMILES: Cc1cncn1Cc1cc(N2Cc3ccc(C4(Cc5nncn5C)COC4)cc3C2=O)nc(C(F)(F)F)c1] | LCMS: 524.2 [M + H]⁺. ¹H NMR (DMSO-d6, 400 MHz): δ 8.43 (s, 1H), 8.21 (s, 1H), 7.73 (s, 1H), 7.64 (d, 1H), 7.51 (d, 1H), 7.39-7.36 (m, 2H), 6.76 (s, 1H), 5.44 (s, 2H), 5.05 (s, 2H), 4.97 (d, 2H), 4.90 (d, 2H), 3.56 (s, 2H), 3.02 (s, 3H), 2.08 (d, 3H). |
| | Starting materials: Int-1 and Int-30 | |
| | Reaction conditions: B | |
| 70 | 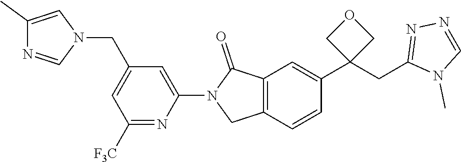 [SMILES: Cc1cn(Cc2cc(N3Cc4ccc(C5(Cc6nncn6C)COC5)cc4C3=O)nc(C(F)(F)F)c2)cn1] | LCMS: 524.1 [M + H]⁺. ¹H NMR (DMSO-d6, 400 MHz): δ 8.57 (s, 1H), 8.21 (s, 1H), 7.68 (d, 1H), 7.65 (d, 1H), 7.51 (d, 1H), 7.48 (s, 1H), 7.38 (dd, 1H), 6.93 (s, 1H), 5.38 (s, 2H), 5.05 (s, 2H), 4.97 (d, 2H), 4.90 (d, 2H), 3.57 (s, 2H), 3.02 (s, 3H), 2.10 (s, 3H). |
| | Starting materials: Int-1 and Int-29 | |
| | Reaction conditions: B | |
| 71 | 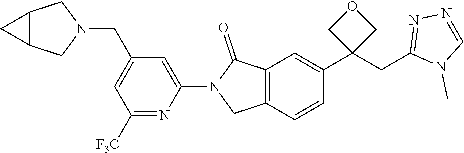 [SMILES: Cn1cnnc1CC1(c2ccc3c(c2)C(=O)N(c2cc(CN4CC5CC5C4)cc(C(F)(F)F)n2)C3)COC1] | LCMS: 525.2 [M + H]⁺. ¹H NMR (400 MHz, DMSO-d6): δ 8.77 (s, 1H), 8.22 (s, 1 H), 7.65 (d, 1H), 7.54 (d, 1H), 7.52 (s, 1H), 7.34 (dd, 1H), 5.06 (s, 2H), 4.98 (d, 2H), 4.91 (d, 2H), 3.79 (s, 2H), 3.58 (s, 2H), 3.10 (s, 3H), 2.96 (d, 2H), 2.41-2.37 (m, 2H), 1.48-1.40 (m, 2H), 0.77-0.73 (m, 1H), 0.47- 0.39 (m, 1H). |
| | Starting materials: Int-1 and Int-86 | |
| | Reaction conditions: B | |
| 72 | 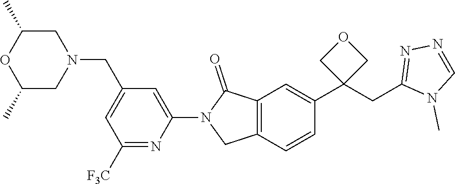 [SMILES: C[C@@H]1CN(Cc2cc(N3Cc4ccc(C5(Cc6nncn6C)COC5)cc4C3=O)nc(C(F)(F)F)c2)C[C@H](C)O1] | LCMS: 557.2 [M + H]⁺. ¹HNMR (400 MHz, DMSO-d6): δ 8.79 (s, 1H), 8.22 (s, 1 H), 7.66 (d, 1H), 7.59 (d, 2H), 7.54 (d, 1H), 7.38 (dd, 1H), 5.07 (s, 2H), 4.98 (d, 2H), 4.91 (d, 2H), 3.65 (s, 2H), 3.64-3.56 (m, 2H), 3.58 (s, 2H), 3.03 (s, 3H), 2.72 (d, 2H), 1.76 (t, 2H), 1.05 (d, 6H). |
| | Starting materials: Int-1 and Int-87 | |
| | Reaction conditions: B | |
| 73 | 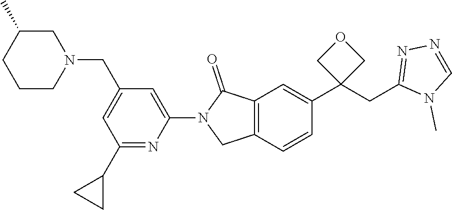 | LCMS: 557.2 [M + HCO₂H]⁻. ¹H NMR (400 MHz, DMSO-d6): δ 8.23 (bs, 1H), 8.21 (s, 1 H), 7.62 (d, 1H), 7.46 (d, 1H), 7.31 (bd, 1H), 7.04 (bs, 1H), 5.01 (s, 2H), 4.98 (d, 2H), 4.90 (d, 2H), 3.57 (s, 2H), 3.49 (s, 2H), 2.99 (s, 3H), 2.78-2.70 (m, 1H), 2.13-2.09 (m, 1H), 2.04-1.98 (m, 1H), 1.98-1.89 (m, 1H), 1.71-1.48 (m, 5H), 1.05-0.91 (m, 4H), 0.90-0.80 (m, 4H). |
| | Starting materials: Int-1 and Int-19 | |
| | Reaction conditions: B | |
| 74 | 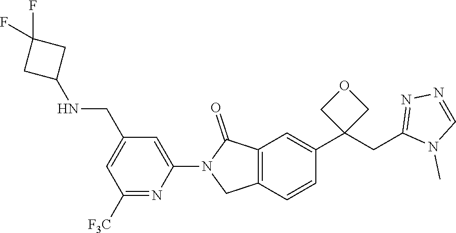 [SMILES: Cn1cnnc1CC1(c2ccc3c(c2)C(=O)N(c2cc(CNC4CC(F)(F)C4)cc(C(F)(F)F)n2)C3)COC1] | LCMS: 549.1 [M + H]⁺. ¹H NMR (400 MHz, DMSO-d6): δ 8.76 (s, 1H), 8.22 (s, 1H), 7.67-7.65 (m, 2H), 7.53 (d, 1H), 7.39 (dd, 1H), 5.07 (s, 2H), 4.98 (d, 2H), 4.91 (d, 2H), 3.83 (d, 2H), 3.58 (s, 2H), 3.21-3.12 (m, 1H), 3.03 (s, 3H), 3.10-2.95 (m, 1H), 2.80-2.70 (m, 2H), 2.48-2.32 (m, 1H). |
| | Starting materials: Int-1 and Int-88 | |
| | Reaction conditions: B | |
| 75 | 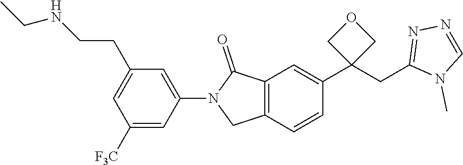 [SMILES: CCNCCc1cc(N2Cc3ccc(C4(Cc5nncn5C)COC4)cc3C2=O)cc(C(F)(F)F)c1] | LCMS: 500.2 [M + H]⁺. ¹H NMR (400 MHz, DMSO-d6): δ 8.24 (brs, 1H), 8.22 (s, 1H), 7.95 (brs, 1H), 7.58 (d, 1H), 7.50 (d, 1H), 7.39 (brs, 1H), 7.34 (dd, 1H), 5.06 (s, 2H), 4.98 (d, 2H), 4.91 (d, 2H), 3.58 (s, 2H), 3.03 (s, 3H), 2.86-2.80 (m, 4H), 2.62-2.55 (m, 2H), 1.01 (t, 3H). |
| | Starting materials: Int-1 and Int-37 | |
| | Reaction conditions: A | |
| 76 | 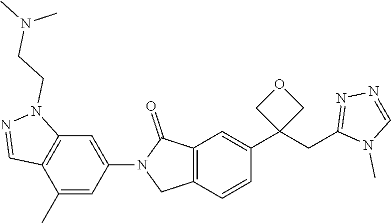 [SMILES: Cc1cc(N2Cc3ccc(C4(Cc5nncn5C)COC4)cc3C2=O)cc2c1cnn2CCN(C)C] | LCMS: 486.2 [M + H]⁺. ¹H NMR (400 MHz, DMSO-d6): δ 8.23 (s, 1H), 8.08 (bs, 1 H), 7.98 (s, 1H), 7.59 (d, 1H), 7.54 (s, 1H), 7.50 (d, 1H), 7.32 (dd, 1H), 5.08 (s, 2H), 4.99 (d, 2H), 4.92 (d, 2H), 4.50-4.40 (m, 2H), 3.59 (s, 2H), 3.03 (s, 3H), 2.75-2.69 (m, 2H), 2.57 (s, 3H), 2.19 (bs, 6H). |
| | Starting materials: Int-1 and Int-20 | |
| | Reaction conditions: A | |
| 77 | 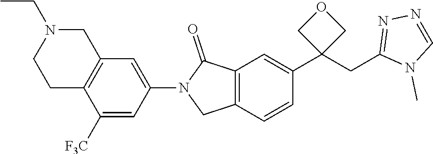 [SMILES: CCN1CCc2c(cc(N3Cc4ccc(C5(Cc6nncn6C)COC5)cc4C3=O)cc2C(F)(F)F)C1] | LCMS: 512.2 [M + H]⁺. ¹H NMR (400 MHz, DMSO-d6): δ 8.25 (brs, 1H), 8.22 (s, 1H), 7.79 (brs, 1H), 7.58 (d, 1H), 7.49 (d, 1H), 7.33 (dd, 1H), 5.02 (s, 2H), 4.98 (d, 2H), 4.90 (d, 2H), 3.66 (s, 2H), 3.58 (s, 2H), 3.03 (s, 3H), 2.92-2.89 (m, 2H), 2.73-2.67 (m, 2H), 2.58-2.53 (m, 2H), 1.12 (t, 3H). |
| | Starting materials: Int-1 and Int-22 | |
| | Reaction conditions: A | |
| 78 | 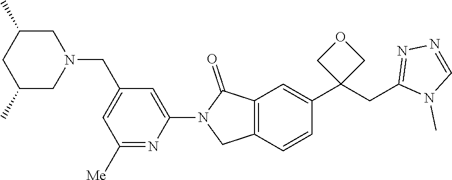 [SMILES: Cc1cc(CN2C[C@H](C)C[C@H](C)C2)cc(N2Cc3ccc(C4(Cc5nncn5C)COC4)cc3C2=O)n1] | LCMS: 501.3 [M + H]⁺. ¹H NMR (DMSO, 400 MHz): δ 8.30 (s, 1H), 8.21 (s, 1H), 7.61 (d, 1H), 7.47 (d, 1H), 7.33 (dd, 1H), 6.98 (s, 1H), 5.06 (s, 2H), 4.98 (d, 2H), 4.91 (d, 2H), 3.57 (s, 2H), 3.46 (s, 2H), 3.00 (s, 3H), 2.79-2.72 (m, 2H), 2.33 (s, 3H), 1.72-1.62 (m, 3H), 1.48 (t, 2H), 0.80 (s, 6H), 0.55-0.48 (m, 1H). |
| | Starting materials: Int-1 and Int-34 | |
| | Reaction conditions: B | |
| 79 | 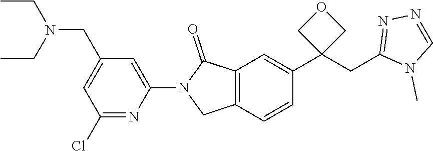 [SMILES: CCN(CC)Cc1cc(Cl)nc(N2Cc3ccc(C4(Cc5nncn5C)COC4)cc3C2=O)c1] | LCMS: 481.1 [M + H]⁺. ¹H NMR (400 MHz, CD₃OD): δ 8.54 (d, 1H), 8.20 (s, 1H), 7.61 (d, 1H), 7.47 (d, 1H), 7.24 (dd, 1H), 7.20 (s, 1H), 5.13 (d, 2H), 5.10 (s, 2H), 5.08 (d, 2H), 3.70 (s, 2H), 3.69 (s, 2H), 2.95 (s, 3H), 2.60 (q, 4H), 1.11 (t, 6H). |
| | Starting materials: Int-1 and Int-35 | |
| | Reaction conditions: B | |
| 80 | 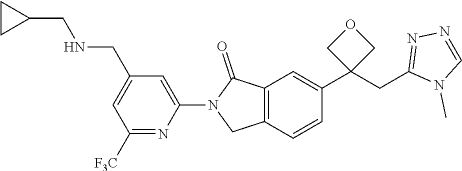 [SMILES: Cn1cnnc1CC1(c2ccc3c(c2)C(=O)N(c2cc(CNCC4CC4)cc(C(F)(F)F)n2)C3)COC1] | LCMS: 513.3 [M + H]⁺. ¹H NMR (400 MHz, DMSO-d6): δ 8.78 (s, 1H), 8.21 (s, 1H), 7.67-7.64 (m, 2H), 7.52 (d, 1H), 7.38 (dd, 1H), 5.07 (d, 2H), 4.98 (s, 2H), 4.91 (d, 2H), 3.90 (s, 2H), 3.58 (s, 2H), 3.32 (s, 3H), 2.40 (d, 2H), 0.95- 0.85 (m, 1H), 0.45-0.39 (m, 2H), 0.15-0.11 (m, 2H). |
| | Starting materials: Int-1 and Int-91 | |
| | Reaction conditions: B | |
| 81 | 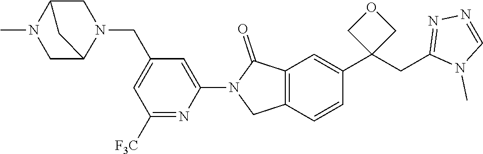 [SMILES: CN1CC2CC1CN2Cc1cc(N2Cc3ccc(C4(Cc5nncn5C)COC4)cc3C2=O)nc(C(F)(F)F)c1] | LCMS: 554.3 [M+H]+. 1H NMR (400 MHz, DMSO-d6): δ 8.84 (s, 1H), 8.22 (s, 1H), 7.66 (d, 1H), 7.59 (s, 1H), 7.53 (d, 1H), 7.38 (dd, 1H), 5.07 (s, 2H), 4.98 (d, 2H), 4.91 (d, 2H), 3.89 (ABq, 2H), 3.58 (s, 2H), 3.30 (s, 1H), 3.18 (s, 1H), 3.03 (s, 3H), 2.78-2.68 (m, 2H), 2.63-2.58 (m, 2H), 2.30 (s, 3H), 1.71-1.62 (m, 2H). |
| | Starting materials: Int-1 and Int-92 | |
| | Reaction conditions: B | |
| 82 | 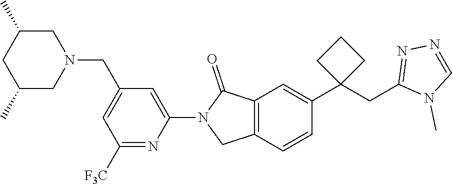 [SMILES: C[C@@H]1C[C@H](C)CN(Cc2cc(N3Cc4ccc(C5(Cc6nncn6C)CCC5)cc4C3=O)nc(C(F)(F)F)c2)C1] | LCMS: 553.2 [M + H]⁺. ¹H NMR (DMSO- d6, 400 MHz): δ 8.78 (s, 1H), 8.15 (s, 1H), 7.60 (d, 1H), 7.57 (d, 1H), 7.35 (d, 1H), 7.23 (dd, 1H), 5.05 (s, 2H), 3.64 (s, 2H), 3.25 (s, 2H), 2.80-2.75 (m, 5H), 2.61- 2.53 (m, 3H), 2.37-2.33 (m, 1H), 2.25- 2.17 (m, 1H), 1.90-1.80 (m, 1H), 1.72- 1.62 (m, 3H), 1.52 (t, 2H), 0.81 (d, 6H), 0.58-0.51 (m, 1H). |
| | Starting materials: Int-3 and Int-62 | |
| | Reaction conditions: B | |
| 83 | 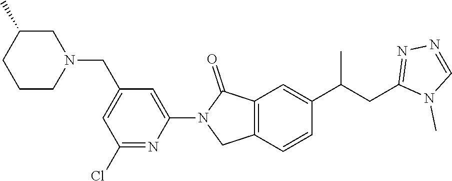 [SMILES: CC(Cc1nncn1C)c1ccc2c(c1)C(=O)N(c1cc(CN3CCC[C@H](C)C3)cc(Cl)n1)C2] | LCMS: 479.3 [M + H]⁺. ¹H NMR (MeOD, 400 MHz): δ 8.60 (s, 1H), 8.36 (brs, 1H), 8.30 (s, 1H), 7.76 (s, 1H), 7.58-7.62 (m, 2H), 7.28 (s, 1H), 5.08 (s, 2H), 3.90 (s, 2H), 3.53 (s, 3H), 3.51-3.46 (m, 1H), 3.23-3.11 (m, 4H), 2.39 (t, 1H), 2.11 (t, 1H), 1.82-1.70 (m, 4H), 1.46 (d, 3H), 1.06-1.04 (m, 1H), 0.95 (d, 3H). |
| | Starting materials: Int-2 and Int-18 | |
| | Reaction conditions: B | |
| 84 | 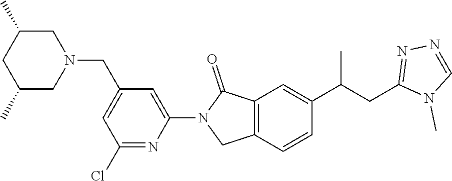 [SMILES: CC(Cc1nncn1C)c1ccc2c(c1)C(=O)N(c1cc(CN3C[C@H](C)C[C@H](C)C3)cc(Cl)n1)C2] | LCMS: 527.4 [M + H]⁺. ¹H NMR (MeOD, 400 MHz): δ 8.83 (s, 1H), 8.29 (s, 1H), 7.78 (s, 1H), 7.61-7.59 (m, 3H), 5.14 (s, 2H), 3.68 (s, 2H), 3.52 (s, 3H), 3.51-348 (m, 1H), 3.23-3.11 (m, 2H), 2.87-2.84 (m, 2H), 1.80-1.76 (m, 2H), 1.66-1.63 (m, 2H), 1.47 (d, 3H), 0.89 (d, 6H) 0.65- 0.55 (m, 1H). |
| | Starting materials: Int-2 and Int-73 | |
| | Reaction conditions: B | |
| 85 | 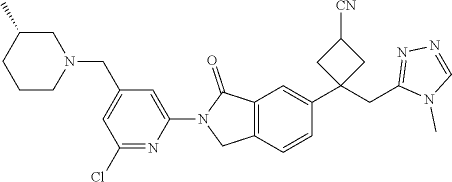 [SMILES: C[C@H]1CCCN(Cc2cc(Cl)nc(N3Cc4ccc(C5(Cc6nncn6C)CC(C#N)C5)cc4C3=O)c2)C1] | LCMS: 532.2 [M + 2 + H]⁺. ¹H NMR (400 MHz, CD₃OD): δ 8.53 (s, 1H), 8.18 (s, 1H), 7.64-7.59 (m, 2H), 7.44- 7.38 (m, 1H), 7.24 (s, 1H), 5.11 (s, 2H), 3.60 (s, 2H), 3.47 (s, 2H), 3.25-3.15 (m, 1H), 3.10-3.05 (m, 2H), 2.87 (s, 3H), 2.86- 2.78 (2 H), 2.10-2.00 (m, 4H), 1.80-1.60 (m, 5H), 1.00-0.85 (m, 4H). |
| | Starting materials: Int-23 and | |
| | Int-18 | |
| | Reaction conditions: B | |
| 86 | 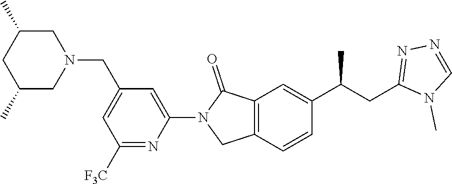 [SMILES: C[C@@H]1C[C@H](C)CN(Cc2cc(N3Cc4ccc([C@@H](C)Cc5nncn5C)cc4C3=O)nc(C(F)(F)F)c2)C1] | LCMS: 527.4 [M + H]⁺. ¹H NMR (MeOD, 400 MHz): δ 8.83 (s, 1H), 8.29 (s, 1H), 7.78 (s, 1H), 7.61-7.59 (m, 3H), 5.14 (s, 2H), 3.68 (s, 2H), 3.52 (s, 3H), 3.51-3.48 (m, 1H), 3.23-3.11 (m, 2H), 2.87-2.84 (m, 2H), 1.79-1.78 (m, 3H), 1.66-1.60 (m, 2H), 1.47 (d, 3H), 0.89 (d, 6H) 0.65-0.53 (m, 1H). |
| | Starting material: Compound 84 | |
| | Reaction conditions: Separated by | |
| | chiral HPLC, Enantiomer 2 | |
| 87 | 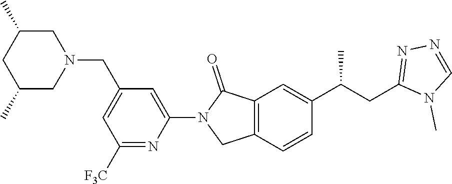 [SMILES: C[C@@H]1C[C@H](C)CN(Cc2cc(N3Cc4ccc([C@H](C)Cc5nncn5C)cc4C3=O)nc(C(F)(F)F)c2)C1] | LCMS: 527.3 [M + H]⁺. ¹H NMR (MeOD, 400 MHz): δ 8.83 (s, 1H), 7.78 (s, 1H), 7.63-7.58 (m, 3H), 5.14 (s, 2H), 3.68 (s, 2H), 3.52 (s, 3H), 3.52-3.46 (m, 1H), 3.21-3.11 (m, 2H), 2.88-2.85 (m, 2H), 1.85-1.70 (m, 3H), 1.63 (t, 2H), 1.47 (d, 3H), 0.89 (d, 6H), 0.62-0.58 (m, 1H). |
| | Starting material: Compound 84 | |
| | Reaction conditions: Separated by | |
| | chiral HPLC, Enantiomer 1 | |
| 88 | 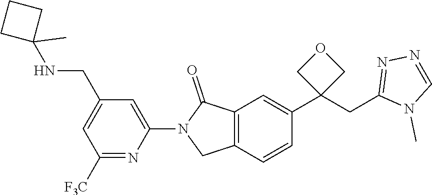 [SMILES: Cn1cnnc1CC1(c2ccc3c(c2)C(=O)N(c2cc(CNC4(C)CCC4)cc(C(F)(F)F)n2)C3)COC1] | LCMS: 527.2 [M + H]⁺. ¹H NMR (400 MHz, DMSO): δ 8.82 (s, 1H), 8.22 (s, 1H), 7.69-7.62 (m, 2H), 7.52 (s, 1H), 7.39 (d, 1H), 5.07 (s, 1H), 4.98 (d, 2H), 4.91 (d, 2H), 3.82 (brs, 2H), 3.58 (s, 2H), 3.51 (s, 1H), 3.03 (s, 3H), 2.05-1.95 (m, 2H), 1.80- 1.60 (m, 4H), 1.24 (brs, 3H). |
| | Staring materials: Int-1 and Int-33 | |
| | Reaction conditions: B | |
| 89 | 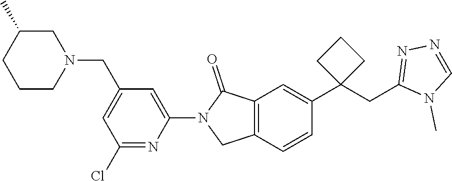 [SMILES: C[C@H]1CCCN(Cc2cc(Cl)nc(N3Cc4ccc(C5(Cc6nncn6C)CCC5)cc4C3=O)c2)C1] | LCMS: 505.4 [M + H]⁺. ¹H NMR (DMSO-d6, 400 MHz): δ 8.49 (d, 1H), 8.16 (s, 1H), 7.57 (dd, 1H), 7.33 (d, 1H), 7.23-7.19 (m, 2H), 5.01 (s, 2H), 3.53 (s, 2H), 3.25 (s, 2H), 2.76 (s, 3H), 2.74-2.70 (m, 2H), 2.58- 2.56 (m, 2H), 2.38-2.31 (m, 2H), 2.23-2.16 (m, 1H), 2.02-1.92 (m, 1H), 1.88-1.72 (m, 1H), 1.70-1.60 (m, 4H), 1.55-1.47 (m, 1H), 0.90-0.82 (m, 4H). |
| | Staring materials: Int-3 and Int-18 | |
| | Reaction conditions: B | |
| 90 | 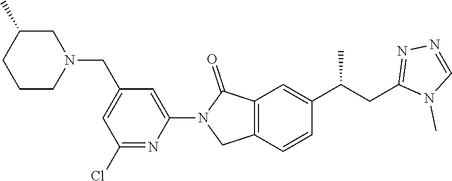 [SMILES: C[C@H]1CCCN(Cc2cc(Cl)nc(N3Cc4ccc([C@H](C)Cc5nncn5C)cc4C3=O)c2)C1] | LCMS: 479.3 [M + H]⁺. ¹H NMR (DMSO-d6, 400 MHz): δ 8.51 (s, 1H), 8.28 (s, 1H), 7.73 (s, 1H), 7.65- 7.60 (m, 2H), 7.20 (d, 1H), 5.02 (s, 2H), 3.54 (s, 2H), 3.49 (s, 3H), 3.44-3.42 (m, 1H), 3.07-3.04 (m, 2H), 2.75-2.70 (m, 2H), 1.98-1.92 (m, 1H), 1.69-1.60 (m, 4H), 1.53-1.49 (m, 1H), 1.34 (d, 3H), 0.86-0.83 (m, 4H). |
| | Staring material: Compound 83 | |
| | Reaction conditions: Separated by | |
| | chiral HPLC, Enantiomer 1 | |
| 91 | 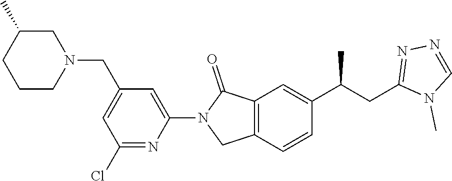 [SMILES: C[C@H]1CCCN(Cc2cc(Cl)nc(N3Cc4ccc([C@@H](C)Cc5nncn5C)cc4C3=O)c2)C1] | LCMS: 479.3 [M + H]⁺. ¹H NMR (DMSO-d6, 400 MHz): δ 8.51 (s, 1H), 8.28 (s, 1H), 7.73 (s, 1H), 7.64- 7.60 (m, 2H), 7.19 (s, 1H), 5.02 (s, 2H), 3.54 (s, 2H), 3.49 (s, 3H), 3.44- 3.42 (m, 1H), 3.07-3.04 (m, 2H), 2.78- 2.70 (m, 2H), 1.99-1.95 (m, 1H), 1.70- 1.69 (m, 4H), 1.58-1.40 (m, 1H), 1.33 (d, 3H), 0.90-0.82 (m, 4H). |
| | Staring materials: Compound 83 | |
| | Reaction conditions: Separated by | |
| | chiral HPLC, Enantiomer 2 | |
| 92 | 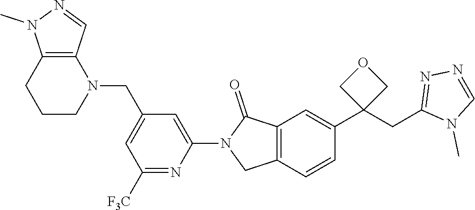 [SMILES: Cn1cnnc1CC1(c2ccc3c(c2)C(=O)N(c2cc(CN4CCCc5c4cnn5C)cc(C(F)(F)F)n2)C3)COC1] | LCMS: 579.2 [M + H]⁺. ¹H NMR (DMSO, 400 MHz): δ 8.83 (s, 1H), 8.37 (s, 1H), 7.68-7.63 (m, 2H), 7.52 (d, 1H), 7.39 (dd, 1H), 6.89 (s, 1H), 5.08 (d, 2H), 4.97 (d, 2H), 4.91 (d, 2H), 4.24 (s, 2H), 3.63 (s, 3H), 3.61 (s, 2H), 3.06 (s, 3H), 2.88-2.84 (m, 2H), 2.64 (t, 2H), 1.99-1.93 (m, 2H). |
| | Staring materials: Int-1 and Int-32 | |
| | Reaction conditions: B | |
| 93 | 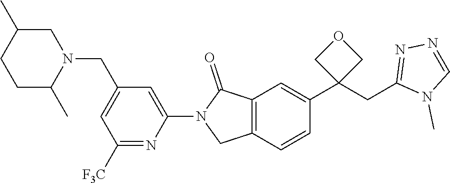 [SMILES: CC1CCC(C)N(Cc2cc(N3Cc4ccc(C5(Cc6nncn6C)COC5)cc4C3=O)nc(C(F)(F)F)c2)C1] | LCMS: 555.4 [M + H]⁺. ¹H NMR (DMSO-d6, 400 MHz): δ 8.84-8.82 (m, 1H), 8.22 (s, 1H), 7.65 (d, 1H), 7.58 (d, 1H), 7.54 (dd, 1H), 7.39- 7.35 (m, 1H), 5.07 (s, 2H), 4.97 (d, 2H), 4.91 (d, 2H), 4.12 (d, 1H), 3.80 (d, 1H), 3.70 (d, 1H), 3.58 (s, 2H), 3.03 (s, 3H), 2. 85-2.78 (m, 0.5H), 2.69-2.65 (m, 0.5H), 2.33-2.21 (m, 2H), 1.72-1.48 (m, 4H), 1.25-1.35 (m, 0.5H), 1.08 (d, 1.5H), 1.03 (d, 1.5H), 1.0-0.9 (m, 0.5H), 0.89 (d, 1.5H), 0.77 (d, 1.5H). |
| | Staring materials: Int-1 and Int-36 | |
| | Reaction conditions: B | |
| 94 | 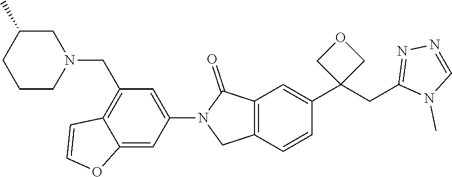 [SMILES: C[C@H]1CCCN(Cc2cc(N3Cc4ccc(C5(Cc6nncn6C)COC5)cc4C3=O)cc3occc23)C1] | LCMS: 512.4 [M+H]+. 1H NMR (DMSO, 400 MHz): δ 8.22 (s, 1H), 8.09 (s, 1H), 7.96 (d, 1H), 7.73 (d, 1H), 7.58 (d, 1H), 7.49 (d, 1H), 7.29 (dd, 1H), 7.09 (dd, 1H), 5.05 (s, 2H), 4.98 (d, 2H), 4.91 (d, 2H), 3.70 (s, 2H), 3.58 (s, 2H), 3.01 (s, 3H), 2.82- 2.75 (m, 2H), 2.03-1.92 (m, 1H), 1.68- 1.42 (m, 5H), 0.90-0.84 (m, 1H) , 0.82 (d, 3H). |
| | Staring materials: Int-1 and Int-38 | |
| | Reaction conditions: A | |

### Example 3. 2-(4-(1H-Pyrazol-4-yl)-6-(trifluoromethyl)pyridin-2-yl)-6-(3-((4-methyl-4H-1,2,4-triazol-3-yl)methyl)oxetan-3-yl)isoindolin-1-one (Compound 95)

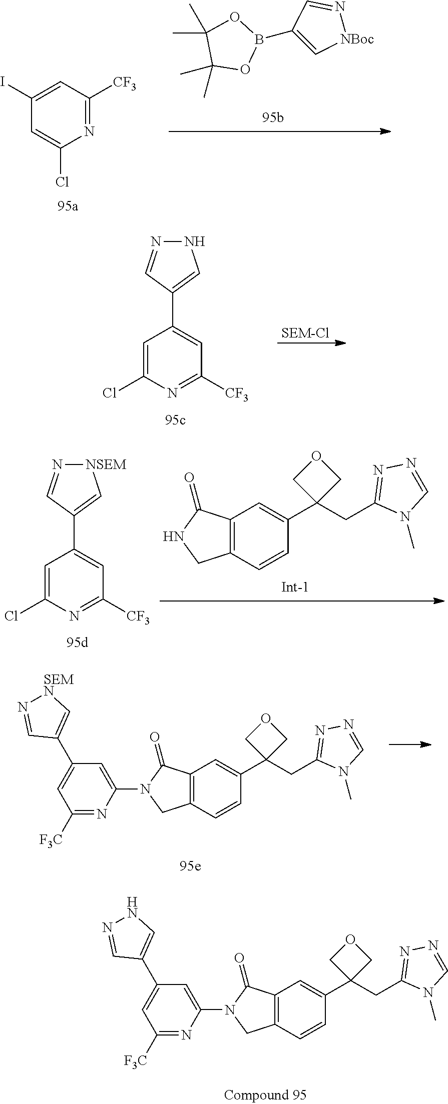

### a) 2-Chloro-4-(1H-pyrazol-4-yl)-6-(trifluoromethyl)pyridine (95c)

To a degassed solution of 2-chloro-4-iodo-6-(trifluoromethyl)pyridine (1.0 g, 3.25 mmol), tert-butyl 4-(4,4,5,5-tetramethyl-1,3,2-dioxaborolan-2-yl)-1H-pyrazole-1-carboxylate (1.05 g, 3.57 mmol) and sodium carbonate (0.86 g, 8.13 mmol) in 1,4 dioxane (20 mL) and water (5 mL) was added Pd(dppf)Cl₂*DCM (0.265 g, 0.325 mmol) followed by stirring at 80° C. for 4 h. The mixture was cooled to RT and filtered through celite pad followed by washing the celite pad with ethyl acetate and concentrating the filtrate. The crude product was purified by silica-gel flash column chromatography to afford the title compound (0.5 g, 62.08%). LCMS: 248.2 [M+H]⁺.

### b) 2-Chloro-6-(trifluoromethyl)-4-(1-((2-(trimethylsilyl)ethoxy)methyl)-1H-pyrazol-4-yl)pyridine (95d)

To a solution of 2-chloro-4-(1H-pyrazol-4-yl)-6-(trifluoromethyl)pyridine (0.4 g, 1.62 mmol) and N,N-diisopropylethylamine (0.41 g, 2.42 mmol) in DCM (8 mL) was added 2-(trimethylsilyl)ethoxymethyl chloride (SEM-C₁) (0.42 mL, 2.42 mmol) at 0° C. followed by stirring at RT for 4 h. The mixture was diluted with water and extracted with DCM. Combined organic layer was dried over anhydrous sodium sulphate, filtered, and concentrated to afford the title compound. (0.4 g, 65.55%). LCMS: 278.2 [M+H]⁺.

### c) 6-(3-((4-Methyl-4H-1,2,4-triazol-3-yl)methyl)oxetan-3-yl)-2-(6-(trifluoro-methyl)-4-(1-((2-(trimethylsilyl)ethoxy)methyl)-1H-pyrazol-4-yl)pyridin-2-yl)iso-indolin-1-one (95e)

The compound was prepared according to the procedure described in Example 2 with appropriate variations in coupling methods, reactants, quantities of reagents and solvents. LCMS: 626.7 [M+H]⁺.

### d) 2-(4-(1H-Pyrazol-4-yl)-6-(trifluoromethyl)pyridin-2-yl)-6-(3-((4-methyl-4H-1,2,4-triazol-3-yl)methyl)oxetan-3-yl)isoindolin-1-one (Compound 95)

To a solution of 6-(3-((4-methyl-4H-1,2,4-triazol-3-yl)methyl)oxetan-3-yl)-2-(6-(trifluoromethyl)-4-(1-((2-(trimethylsilyl)ethoxy)methyl)-1H-pyrazol-4-yl)pyridin-2-yl)isoindolin-1-one (0.05 g, 0.08 mmol) in DCM (5 mL) was added TFA followed by stirring at RT for 12 h. The mixture was concentrated and purified by silica-gel flash column chromatography using 10% methanol in chloroform as eluent to afford the title compound (0.033 g, 83.26%); LCMS: 496.05 [M+H]⁺. ¹H NMR (DMSO-d6, 400 MHz): δ 13.37 (s, 1H), 8.92 (d, 1H), 8.60. (s, 1H), 8.22 (s, 1H), 8.17 (s, 1H), 7.93 (d, 1H), 7.67 (d, 1H), 7.52 (d, 1H), 7.40 (dd, 1H), 5.09 (s, 2H), 4.99 (d, 2H), 4.92 (d, 2H), 3.58 (s, 2H), 3.03 (s, 3H).

### Example 4. (R)-6-(3-((4-Methyl-4H-1,2,4-triazol-3-yl)methyl)oxetan-3-yl)-2-(4-((3-methylpiperazin-1-yl)methyl)-6-(trifluoromethyl)pyridin-2-yl)isoindolin-1-one (Compound 96) and (R)-2-(4-((3,4-Dimethylpiperazin-1-yl)methyl)-6-(trifluoromethyl)-pyridin-2-yl)-6-(3-((4-methyl-4H-1,2,4-triazol-3-yl)methyl)oxetan-3-yl)isoindolin-1-one (Compound 97)

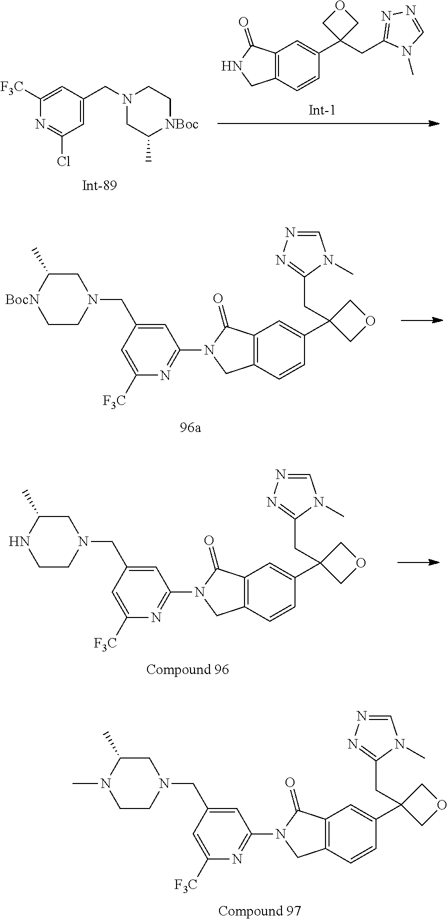 [SMILES: C[C@@H]1CN(Cc2cc(Cl)nc(C(F)(F)F)c2)CCN1C(=O)OC(C)(C)C ; Cn1cnnc1CC1(c2ccc3c(c2)C(=O)NC3)COC1 ; C[C@@H]1CN(Cc2cc(N3Cc4ccc(C5(Cc6nncn6C)COC5)cc4C3=O)nc(C(F)(F)F)c2)CCN1C(=O)OC(C)(C)C ; C[C@@H]1CN(Cc2cc(N3Cc4ccc(C5(Cc6nncn6C)COC5)cc4C3=O)nc(C(F)(F)F)c2)CCN1 ; C[C@@H]1CN(Cc2cc(N3Cc4ccc(C5(Cc6nncn6C)COC5)cc4C3=O)nc(C(F)(F)F)c2)CCN1C]

### a) tert-Butyl (R)-2-methyl-4-((2-(6-(3-((4-methyl-4H-1,2,4-triazol-3-yl)methyl)-oxetan-3-yl)-1-oxoisoindolin-2-yl)-6-(trifluoromethyl)pyridin-4-yl)methyl)piperazine-1-carboxylate (96a)

The compound was prepared according to procedure described in Example 2 with appropriate variations in coupling methods, reactants, quantities of reagents, and solvents. LCMS: 642.2 [M+H]⁺.

### b) (R)-6-(3-((4-Methyl-4H-1,2,4-triazol-3-yl)methyl)oxetan-3-yl)-2-(4-((3-methylpiperazin-1-yl)methyl)-6-(trifluoromethyl)pyridin-2-yl)isoindolin-1-one (Compound 96)

To a solution of tert-butyl (R)-2-methyl-4-((2-(6-(3-((4-methyl-4H-1,2,4-triazol-3-yl)methyl)oxetan-3-yl)-1-oxoisoindolin-2-yl)-6-(trifluoromethyl)pyridin-4-yl)-methyl)piperazine-1-carboxylate (0.23 g, 0.35 mmol) in DCM (2 mL) was added TFA (0.5 mL) at 0° C. The mixture was slowly warmed to RT and stirred for 1 h. The mixture was concentrated, basified with aqueous bicarbonate solution and extracted with DCM. The combined organic layer was dried over anhydrous sodium sulphate, filtered, concentrated and purified by silica-gel preparative TLC to afford the title compound (0.19 g, 98%). LCMS: 542.2 [M+H]⁺. ¹H NMR (DMSO-d6, 400 MHz): δ 8.79 (s, 1H), 8.22 (s, 1H), 7.66 (d, 1H), 7.58 (s, 1H), 7.53 (d, 1H), 7.38 (dd, 1H), 5.07 (s, 2H), 4.98 (d, 2H), 4.91 (d, 2H), 3.63 (s, 2H), 3.58 (s, 2H), 3.03 (s, 3H), 2.85-2.81 (m, 1H), 2.78-2.72 (m, 2H), 2.71-2.63 (m, 2H), 2.01-1.98 (m, 1H), 1.68 (t, 1H), 0.92 (d, 3H).

### c) (R)-2-(4-((3,4-Dimethylpiperazin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 97)

To a solution of (R)-6-(3-((4-methyl-4H-1,2,4-triazol-3-yl)methyl)oxetan-3-yl)-2-(4-((3-methylpiperazin-1-yl)methyl)-6-(trifluoromethyl)pyridin-2-yl)isoindolin-1-one (0.1 g, 0.18 mmol) in MeOH (3 mL) was added formic acid (0.03 g, 0.74 mmol) and formaldehyde (0.05 g, 1.85 mmol) followed by stirring at 70° C. for 6 h. The mixture was cooled to RT, concentrated, basified with aqueous bicarbonate solution, and extracted with ethyl acetate. The combined organic layer was dried over anhydrous sodium sulphate, filtered, concentrated, and purified by silica-gel preparative TLC using 7% MeOH in DCM to afford the title compound (0.018 g, 17.51%). LCMS: 556.2[M+H]⁺. ¹H NMR (DMSO-d6, 400 MHz): δ 8.80 (s, 1H), 8.22 (s, 1H), 7.65 (d, 1H), 7.58 (d, 1H), 7.54 (d, 1H), 7.38 (dd, 1H), 5.07 (s, 2H), 4.98 (d, 2H), 4.91 (d, 2H), 3.70-3.61 (m, 2H), 3.57 (s, 2H), 3.03 (s, 3H), 2.69-2.66 (m, 3H), 2.22-2.18 (m, 5H), 2.02-1.98 (m, 1H), 1.89 (t, 1H), 0.95 (d, 3H).

### Example 5. 2-(4-(((3R,5S)-3,5-Dimethylpiperazin-1-yl)methyl)-6-(trifluoro-methyl)pyridin-2-yl)-6-(3-((4-methyl-4H-1,2,4-triazol-3-yl)methyl)oxetan-3-yl)-isoindolin-1-one (Compound 98)

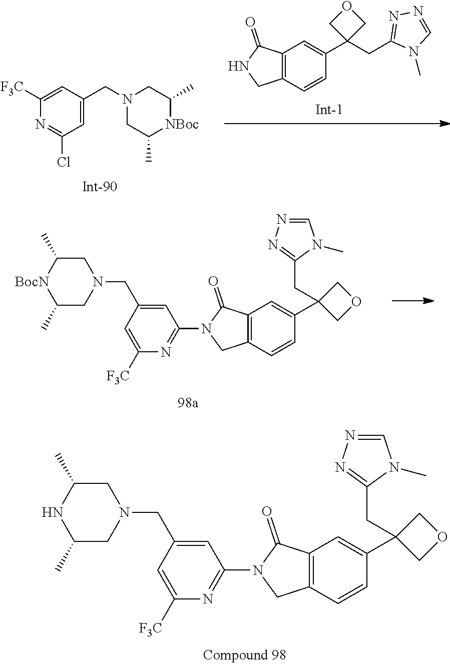 [SMILES: C[C@@H]1CN(Cc2cc(Cl)nc(C(F)(F)F)c2)C[C@H](C)N1C(=O)OC(C)(C)C ; Cn1cnnc1CC1(c2ccc3c(c2)C(=O)NC3)COC1 ; C[C@@H]1CN(Cc2cc(N3Cc4ccc(C5(Cc6nncn6C)COC5)cc4C3=O)nc(C(F)(F)F)c2)C[C@H](C)N1C(=O)OC(C)(C)C ; C[C@@H]1CN(Cc2cc(N3Cc4ccc(C5(Cc6nncn6C)COC5)cc4C3=O)nc(C(F)(F)F)c2)C[C@H](C)N1]

### a) tert-Butyl (2R,6S)-2,6-dimethyl-4-((2-(6-(3-((4-methyl-4H-1,2,4-triazol-3-yl)methyl)oxetan-3-yl)-1-oxoisoindolin-2-yl)-6-(trifluoromethyl)pyridin-4-yl)methyl)-piperazine-1-carboxylate (98a)

The compound was prepared according to the procedure described in Example 2 with appropriate variations in coupling methods, reactants, quantities of reagents, and solvents. LCMS: 656.2 [M+H]⁺.

### b) 2-(4-(((3R,5S)-3,5-Dimethylpiperazin-1-yl)methyl)-6-(trifluoromethyl)-pyridin-2-yl)-6-(3-((4-methyl-4H-1,2,4-triazol-3-yl)methyl)oxetan-3-yl)isoindolin-1-one (Compound 98)

The compound was prepared according to the procedure described in Example 4 with appropriate variations in coupling methods, reactants, quantities of reagents, and solvents. LCMS: 556.2 [M+H]⁺. ¹H NMR (DMSO-d6, 400 MHz): δ 8.78 (s, 1H), 8.22 (s, 1H), 7.66 (d, 1H), 7.57 (s, 1H), 7.53 (d, 1H), 7.38 (dd, 1H), 5.07 (s, 2H), 4.98 (d, 2H), 4.91 (d, 2H), 3.63 (s, 2H), 3.58 (s, 2H), 3.03 (s, 3H), 2.88-2.75 (m, 2H), 2.72-2.62 (m, 2H), 1.61 (t, 2H), 0.92 (d, 6H).

### Example 6. 2-(4-(13-Amino-5,8,11-trioxa-2-azatridecyl)-6-chloropyridin-2-yl)-6-(3-((4-methyl-4H-1,2,4-triazol-3-yl)methyl)oxetan-3-yl)isoindolin-1-one (Compound 99)

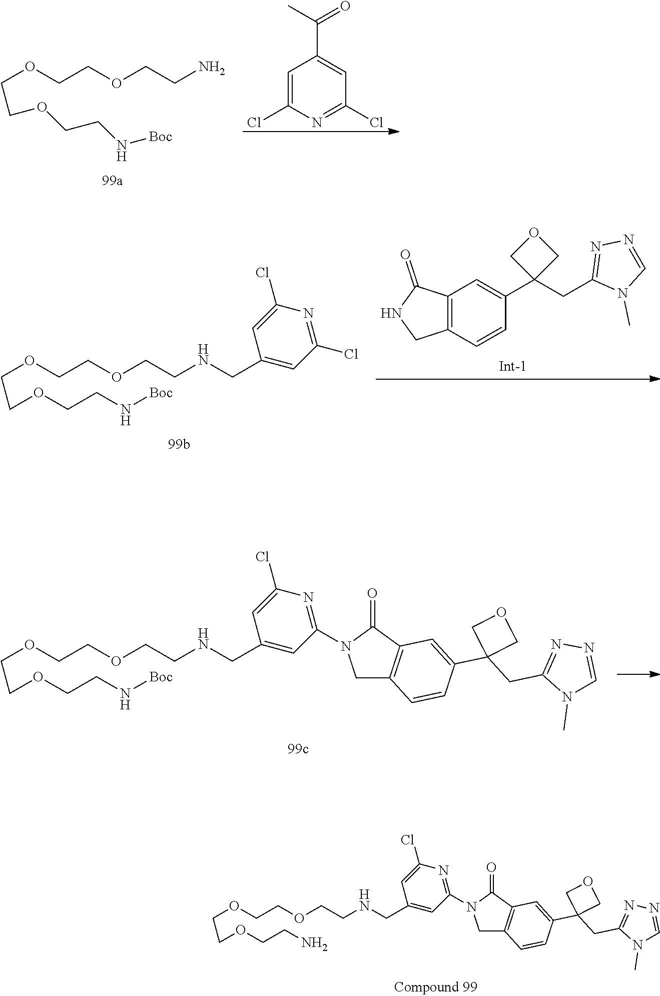 [SMILES: CC(C)(C)OC(=O)NCCOCCOCCOCCN ; CC(=O)c1cc(Cl)nc(Cl)c1 ; CC(C)(C)OC(=O)NCCOCCOCCOCCNCc1cc(Cl)nc(Cl)c1 ; Cn1cnnc1CC1(c2ccc3c(c2)C(=O)NC3)COC1 ; Cn1cnnc1CC1(c2ccc3c(c2)C(=O)N(c2cc(CNCCOCCOCCOCCNC(=O)OC(C)(C)C)cc(Cl)n2)C3)COC1 ; Cn1cnnc1CC1(c2ccc3c(c2)C(=O)N(c2cc(CNCCOCCOCCOCCN)cc(Cl)n2)C3)COC1]

### a) tert-Butyl (1-(2,6-dichloropyridin-4-yl)-5,8,11-trioxa-2-azatridecan-13-yl) carbamate (99b)

To a solution of tert-butyl (2-(2-(2-(2-aminoethoxy) ethoxy)ethoxy)ethyl) carbamate (0.40 g, 1.37 mmol) and 2,6-dichloroisonicotinaldehyde (0.60 g, 3.42 mmol) in DCM (4.0 mL) was added acetic acid (0.1 mL) at 0° C. The mixture was warmed to RT and followed by stirring for 12 h. Then STAB (0.58 g, 2.73 mmol) was added to the mixture followed by stirring for 6 h. The mixture was diluted with aqueous sodium bicarbonate and extracted with in DCM. The combined organic layer was dried over anhydrous sodium sulphate, filtered, concentrated and purified by silica-gel flash column chromatography using 0-20% methanol in DCM as eluent to afford the title compound (0.390 g, 63.02%). LCMS: 454.0 [M+H]⁺.

### b) tert-Butyl(1-(2-chloro-6-(6-(3-((4-methyl-4H-1,2,4-triazol-3-yl) methyl)-oxetan-3-yl)-1-oxoisoindolin-2-yl)pyridin-4-yl)-5,8,11-trioxa-2-azatridecan-13-yl)-carbamate (99c)

To a degassed solution of tert-butyl (1-(2,6-dichloropyridin-4-yl)-5,8,11-trioxa-2-azatridecan-13-yl)carbamate (0.33 g, 0.73 mmol), 6-(3-((4-methyl-4H-1,2,4-triazol-3-yl)methyl)oxetan-3-yl)isoindolin-1-one (0.21 g, 0.73 mmol), Cs₂CO₃ (0.71 g, 2.18 mmol) and Xantphos (0.084 g, 0.140 mmol) in DMSO (3.3 mL) was added Pd(OAc)₂ (0.016 g, 0.07 mmol) followed by stirring at 80° C. for 1 h. The mixture was cooled to RT and passed through celite bed which was washed with 10% methanol in DCM. The filtrate was diluted with water and extracted with 10% methanol in DCM. The organic layer was washed with brine, dried over anhydrous sodium sulphate, filtered, concentrated, and purified by silica-gel flash column chromatography using 0-20% methanol in DCM as eluent to afford the title compound (0.135 g, 26.45%). LCMS: 701.3 [M+H]⁺.

### c) 2-(4-(13-Amino-5,8,11-trioxa-2-azatridecyl)-6-chloropyridin-2-yl)-6-(3-((4-methyl-4H-1,2,4-triazol-3-yl) methyl)oxetan-3-yl)isoindolin-1-one (Compound 99)

To a solution of tert-butyl (1-(2-chloro-6-(6-(3-((4-methyl-4H-1,2,4-triazol-3-yl)methyl)oxetan-3-yl)-1-oxoisoindolin-2-yl)pyridin-4-yl)-5,8,11-trioxa-2-azatridecan-13-yl)carbamate (0.065 g, 0.093 mmol) in DCM (2.0 mL) was added trifluoroacetic acid (0.11 g, 0.93 mmol) at 0° C. The mixture was slowly warmed to RT followed by stirring for 4 h. The mixture was concentrated and triturated with diethyl ether. The crude mixture was dissolved by 10% MeOH in DCM, and bicarbonate anion exchange resin was added followed by stirring for 12 h at RT. The resin was filtered, concentrated, and purified by preparative HPLC to afford the title compound (0.019 g, yield: 34.04%). LCMS: 600.15 [M+H]⁺. ¹H NMR ((400 MHz, DMSO): δ 8.47 (d, 1H), 8.30 (s, 2H), 8.22 (s, 1H), 7.63 (d, 1H), 7.49 (d, 1H), 7.40 (dd, 1H), 7.26 (d, 1H), 5.03 (s, 2H), 4.98 (d, 2H), 4.91 (d, 2H), 3.82 (s, 2H), 3.58-3.54 (m, 9H), 3.51 (t, 3H), 3.04 (s, 3H), 2.90 (t, 2H), 2.68-2.66 (m, 2H).

### Example 7. 2-(3-(1-Hydroxy-2-(isopropylamino)ethyl)-5-(trifluoromethyl)-phenyl)-6-(3-((4-methyl-4H-1,2,4-triazol-3-yl)methyl)oxetan-3-yl)isoindolin-1-one (Compound 100)

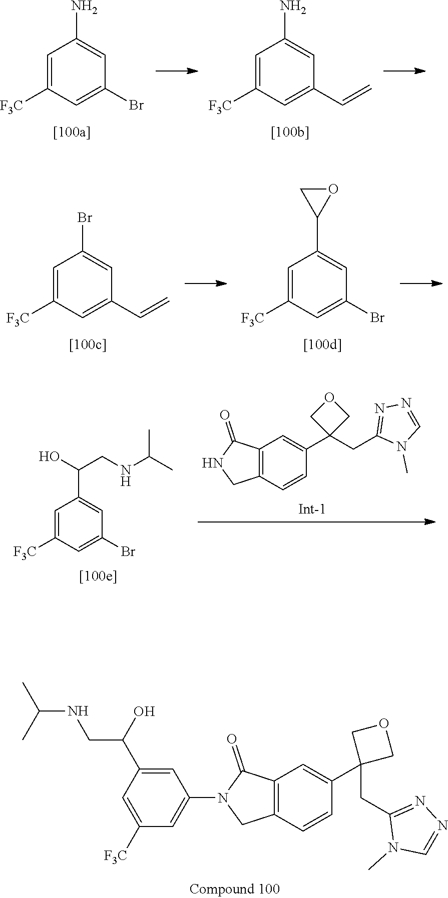

### a) 3-(Trifluoromethyl)-5-vinylaniline [100b]

To a degassed solution of 3-bromo-5-(trifluoromethyl)aniline (15.0 g, 62.49 mmol), potassium vinyl trifluoroborate (7.88 g, 187.48 mmol) and Cs₂CO₃ (50.90 g, 156.23 mmol) in 1,4-dioxane (100 mL) and water (20 mL) was added Pd(dppf)Cl₂*DCM followed by stirring at 100° C. for 6 h. The mixture was cooled to RT, quenched with water and extracted with DCM. Then organic layer was dried over sodium sulphate, filtered, and concentrated to afford the crude product. The crude product was purified by silica-gel flash column chromatography using 10-20% ethyl acetate in hexane as eluent to afford the title compound (8.0 g, 68.40%) LCMS: 188.0 [M+H]⁺.

### b) 1-Bromo-3-(trifluoromethyl)-5-vinylbenzene [100c]

To a solution of 3-(trifluoromethyl)-5-vinylaniline (0.5 g, 2.67 mmol) and cupric bromide (0.60 g, 2.67 mmol) in acetonitrile (8 mL) was added tert-butyl nitrite at 0° C. followed by stirring for 30 min. The mixture was filtered through celite, and filtrate was concentrated to afford the crude product. The crude product was purified by silica-gel flash column chromatography using 5-20% ethyl acetate in hexane as eluent to afford the title compound (0.2 g, 29.83%). ¹H NMR ((400 MHz, DMSO): δ 8.06 (s, 1H), 7.87 (s, 1H), 7.85 (s, 1H), 6.79 (dd, 1H), 6.09 (d, 1H), 5.43 (d, 1H).

### c) 2-(3-Bromo-5-(trifluoromethyl)phenyl)oxirane [100d]

To a solution of 1-bromo-3-(trifluoromethyl)-5-vinylbenzene (0.25 g, 1.0 mmol) in DCM (2 mL) was added meta-chloroperoxybenzoic acid (MCPBA) (0.26 g, 1.4 mmol) at 0° C. The mixture was warmed to RT and stirred for 16 h. The mixture was quenched by saturated sodium bicarbonate and extracted with ethyl acetate. The organic layer was dried over sodium sulphate and concentrated to afford the crude product. The crude product was purified by silica gel flash column chromatography using 10-30% ethyl acetate in hexane as eluent to afford the title compound (0.170 g, 63.92%). 1H NMR ((400 MHz, DMSO): δ 7.92 (s, 1H), 7.82 (s, 1H), 7.69 (s, 1H), 4.12-4.10 (m, 1H), 3.19-3.16 (m, 1H), 2.99-2.96 (m, 1H).

### e) 1-(3-Bromo-5-(trifluoromethyl)phenyl)-2-(isopropyl amino)ethan-1-ol [100e]

To a solution of 2-(3-bromo-5-(trifluoromethyl)phenyl)oxirane (0.10 g, 0.37 mmol) in methanol (2 mL) was added isopropyl amine (0.022 g, 0.37 mmol) followed by stirring at 80° C. for 1 h. The mixture was concentrated to afford the title compound (0.095 g, 77.88%). LC-MS: 328.0 [M+2+H]⁺.

### f) 2-(3-(1-Hydroxy-2-(isopropylamino) ethyl)-5-(trifluoromethyl)phenyl)-6-(3-((4-methyl-4H-1,2,4-triazol-3-yl)methyl)oxetan-3-yl)isoindolin-1-one (Compound 100)

The compound was prepared according to the procedure described in Example 2 with appropriate variations in coupling methods, reactants, quantities of reagents and solvents. LCMS: 530.2 [M+H]⁺. 1H NMR ((400 MHz, CD₃OD): δ 8.67 (s, 1H), 8.30 (s, 1H), 8.22 (s, 1H), 7.67-7.64 (m, 2H), 7.54 (s, 1H), 7.39 (dd, 1H), 5.07-5.14 (m, 7H), 3.80 (s, 2H), 3.54-3.47 (m, 1H), 3.37-3.35 (m, 2H), 3.25-3.15 (m, 1H), 3.17 (s, 3H), 1.40 (t, 6H, two doublet partially overlapped).

### Example 8. 6-(4-Chloro-2-(4-methyl-4H-1,2,4-triazol-3-yl)phenyl)-2-(4-(((cyclopropylmethyl)amino)methyl)-6-methylpyridin-2-yl)isoindolin-1-one (Compound 101)

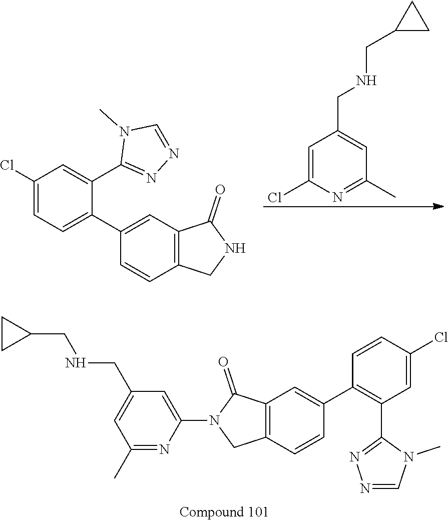 [SMILES: Cn1cnnc1-c1cc(Cl)ccc1-c1ccc2c(c1)C(=O)NC2 ; Cc1cc(CNCC2CC2)cc(Cl)n1 ; Cc1cc(CNCC2CC2)cc(N2Cc3ccc(-c4ccc(Cl)cc4-c4nncn4C)cc3C2=O)n1]

To a degassed solution of 6-(4-chloro-2-(4-methyl-4H-1,2,4-triazol-3-yl) phenyl)isoindolin-1-one (0.30 g, 0.92 mmol), 1-(2-chloro-6-methylpyridin-4-yl)-N-(cyclopropylmethyl)methanamine (0.234 g, 1.11 mmol), Cs₂CO₃ (0.90 g, 2.77 mmol) and XantPhos (0.08 g, 0.14 mmol) in DMSO (3.0 mL) was added Pd(OAc)₂ (0.02 g, 0.09 mmol) followed by stirring at 100° C. for 6 h. The mixture was cooled to RT, filtered through celite pad and washed with 10% MeOH in DCM. The filtrate was washed with brine, dried over anhydrous sodium sulphate, concentrated and purified by silica-gel flash column chromatography using 8% MeOH in DCM as eluent to afford the title compound as an off-white solid (0.024 g). LCMS: 499.2 [M+H]⁺. ¹H NMR (400 MHz, DMSO-d6): δ 8.44 (s, 1H), 8.31 (s, 1H), 7.79 (dd, 1H), 7.72-7.70 (m, 2H), 7.65 (d, 1H), 7.52 (d, 1H), 7.39 (dd, 1H), 7.04 (s, 1H), 5.09 (s, 2H), 3.76 (s, 2H), 3.12 (s, 3H), 2.45 (s, 3H), 2.39 (d, 2H), 0.92-0.86 (m, 1H), 0.43-0.38 (m, 2H), 0.13-0.09 (m, 2H).

### Example 9. 6-(4-Chloro-2-(4-methyl-4H-1,2,4-triazol-3-yl)phenyl)-2-(6-chloro-4-(((cyclopropylmethyl)amino)methyl)pyridin-2-yl)isoindolin-1-one (Compound 102)

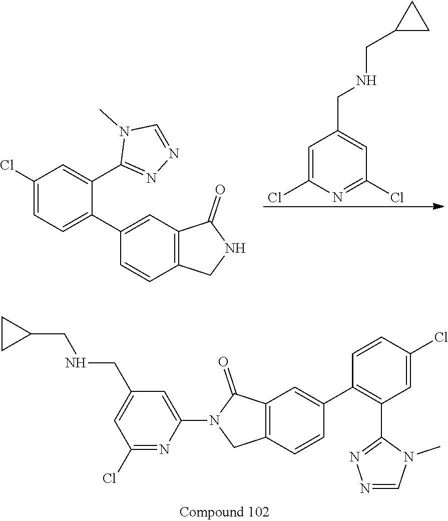 [SMILES: Cn1cnnc1-c1cc(Cl)ccc1-c1ccc2c(c1)C(=O)NC2 ; Clc1cc(CNCC2CC2)cc(Cl)n1 ; Cn1cnnc1-c1cc(Cl)ccc1-c1ccc2c(c1)C(=O)N(c1cc(CNCC3CC3)cc(Cl)n1)C2]

To a degassed solution of 6-(4-chloro-2-(4-methyl-4H-1,2,4-triazol-3-yl)-phenyl)isoindolin-1-one (0.30 g, 0.94 mmol), 1-cyclopropyl-N-((2,6-dichloropyridin-4-1) methyl) methanamine (0.28 g, 1.20 mmol), Cs₂CO₃ (0.90 g, 2.78 mmol) and Xantphos (0.05 g, 0.09 mmol) in dioxane (5 ml) was added Pd₂(dba)₃ (0.08 g, 0.09 mmol) followed by stirring at 90° C. for 6 h. The mixture was cooled to RT, filtered through celite pad and washed with 10% MeOH in DCM. The filtrate was washed with brine, dried over anhydrous sodium sulphate, concentrated, and purified by silica-gel flash column chromatography using 8% MeOH in DCM as eluent to afford the title compound as an off-white solid (0.03 g). LCMS: 519.2 [M+H]⁺.

### Example 10. 6-(4-Chloro-2-(4-methyl-4H-1,2,4-triazol-3-yl) phenyl)-2-(4-(1-((cyclopropylmethyl)amino)ethyl)-6-methylpyridin-2-yl)isoindolin-1-one (Compound 103)

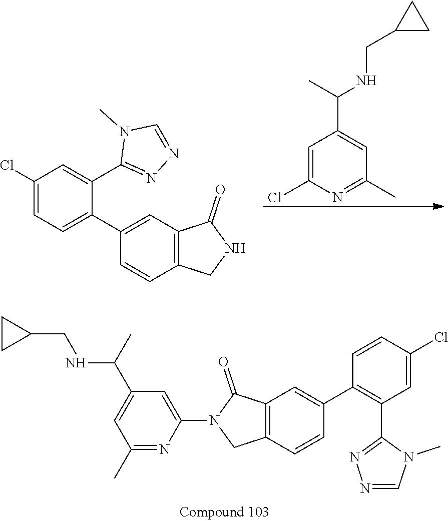 [SMILES: Cn1cnnc1-c1cc(Cl)ccc1-c1ccc2c(c1)C(=O)NC2 ; Cc1cc(C(C)NCC2CC2)cc(Cl)n1 ; Cc1cc(C(C)NCC2CC2)cc(N2Cc3ccc(-c4ccc(Cl)cc4-c4nncn4C)cc3C2=O)n1]

To a degassed solution of 6-(4-chloro-2-(4-methyl-4H-1,2,4-triazol-3-yl)-phenyl)isoindolin-1-one (0.30 g, 0.92 mmol), 1-(2-chloro-6-methylpyridin-4-yl)-N-(cyclopropylmethyl)ethan-1-amine (0.31 g, 1.38 mmol), Cs₂CO₃ (0.90 g, 2.77 mmol) and DavePhos (0.07 g, 0.18 mmol) in DMSO (6.0 mL) was added Pd(OAc)₂ (0.02 g, 0.09 mmol) followed by stirring at 100° C. for 2 h. The mixture was cooled to RT, filtered through celite pad and washed with DCM. The filtrate was washed with brine, dried over anhydrous sodium sulphate, concentrated, and purified by silica-gel flash column chromatography using 5% MeOH in DCM as eluent to afford the title compound as an off-white solid. The mixture was further purified by preparative TLC using 5% methanol in dichloromethane as eluent to afford the title compound (0.05 g, 10.63%). LCMS: 513.2 [M+H]⁺. ¹H NMR (400 MHz, DMSO-d6): δ 8.44 (s, 1H), 8.30 (s, 1H), 7.80 (dd, 1H), 7.72-7.70 (m, 2H), 7.66 (dd, 1H), 7.51 (d, 1H), 7.40 (dd, 1H), 7.05 (s, 1H), 5.08 (s, 2H), 3.75 (q, 1H), 3.11 (s, 3H), 2.45 (s, 3H), 2.27-2.17 (m, 2H), 1.26 (d, 3H), 0.90-0.82 (m, 1H), 0.41-0.32 (m, 2H), 0.07-0.03 (m, 2H).

The following examples were prepared according to the procedure described for Example 8 (reaction conditions A′), Example 9 (reaction conditions B′) or Example 10 (reaction conditions C′) with appropriate variations in reactants, quantities of reagents, coupling methods and solvents. The characterization data of the compounds are also summarized in the Table.

| | Structure, starting material and | |
|---|---|---|
| No. | reaction conditions | Characterization data |
| 104 | 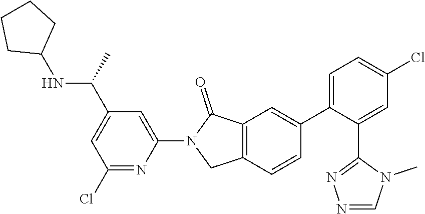 [SMILES: C[C@@H](NC1CCCC1)c1cc(Cl)nc(N2Cc3ccc(-c4ccc(Cl)cc4-c4nncn4C)cc3C2=O)c1] | LCMS: 547.2 [M + 1H]⁺. ¹H NMR (400 MHz, DMSO-d6): δ 8.48 (d, 1H), 8.44 (s, 1H), 7.80 (dd, 1H), 7.72-7.70 (m, 2H), 7.67 (dd, 1H), 7.52 (d, 1H), 7.43 (dd, 1H), 7.32 (d, 1H), 5.06 (s, 2H), 3.84-3.78 (m, 1H), 3.12 (s, 3H), 2.81- 2.75 (m, 1H), 1.69-1.53 (m, 4H), 1.45- 1.30 (m, 3H), 1.29-1.22 (m, 4H). |
| | Starting materials: Int-93 and Int-112. | |
| | Reaction conditions: A′. | |
| | Single enantiomer of unknown | |
| | absolute configuration, separated by | |
| | chiral HPLC. | |
| 105 | 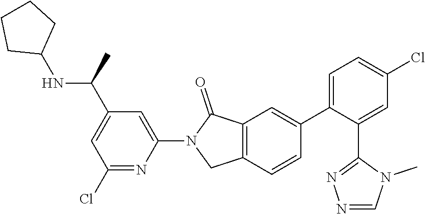 [SMILES: C[C@H](NC1CCCC1)c1cc(Cl)nc(N2Cc3ccc(-c4ccc(Cl)cc4-c4nncn4C)cc3C2=O)c1] | LCMS: 547.2 [M + 1H]⁺. ¹H NMR (400 MHz, DMSO-d6): δ 8.48 (d, 1H), 8.44 (s, 1H), 7.80 (dd, 1H), 7.72-7.69 (m, 2H), 7.66 (d, 1H), 7.52 (d, 1H), 7.43 (dd, 1H), 7.32 (d, 1H), 5.06 (s, 2H), 3.84-3.78 (m, 1H), 3.12 (s, 3H), 2.82- 2.74 (m, 1H), 1.70-1.52 (m, 4H), 1.48- 1.30 (m, 3H), 1.29-1.21 (m, 4H). |
| | Starting materials: Int-93 and Int-112. | |
| | Reaction conditions: A′. | |
| | Single enantiomer of unknown | |
| | absolute configuration, separated by | |
| | chiral HPLC. | |
| 106 | 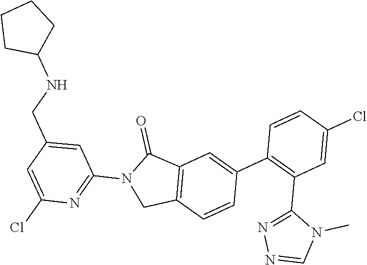 [SMILES: Cn1cnnc1-c1cc(Cl)ccc1-c1ccc2c(c1)C(=O)N(c1cc(CNC3CCCC3)cc(Cl)n1)C2] | LCMS: 533.2 [M + H]⁺; ¹H NMR (400 MHz, CD₃OD): δ 8.51 (d, 1H), 8.40 (s, 1H), 7.80 (dd, 1H), 7.73-7.70 (m, 3H), 7.64 (dd, 1H), 7.48 (dd, 1H), 7.27 (d, 1H), 5.12 (s, 2H), 3.86 (s, 2H), 3.19 (s, 3H), 3.18-3.10 (m, 1H), 1.98- 1.89 (m, 2H), 1.81-1.70 (m, 2H), 1.63-1.53 (m, 2H), 1.49-1.39 (m, 2H). |
| | Starting materials: Int-93 and Int-108. | |
| | Reaction conditions: A′ | |
| 107 | 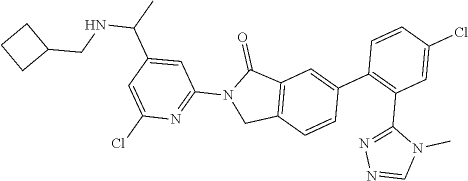 [SMILES: CC(NCC1CCC1)c1cc(Cl)nc(N2Cc3ccc(-c4ccc(Cl)cc4-c4nncn4C)cc3C2=O)c1] | LCMS: 547.2 [M + H]⁺. ¹H-NMR (400 MHz, DMSO-d6): δ 8.47 (s, 1H), 8.44 (s, 1H), 7.80 (dd, 1H), 7.78-7.70 (m, 2H), 7.66 (d, 1H), 7.53 (d, 1H), 7.42 (dd, 1H), 7.29 (s, 1H), 5.06 (s, 2H), 3.74 (q, 1H), 3.12 (s, 3H), 2.48-2.41 (m, 1H), 2.40-2.28 (m, 2H), 2.04-1.92 (m, 2H), 1.85-1.70 (m, 2H), 1.68-1.55 (m, 2H), 1.26 (d, 3H). |
| | Starting materials: Int-93 and Int-113. | |
| | Reaction conditions: A′ | |
| 108 | 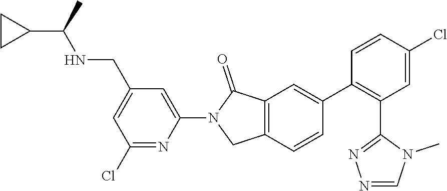 [SMILES: C[C@@H](NCc1cc(Cl)nc(N2Cc3ccc(-c4ccc(Cl)cc4-c4nncn4C)cc3C2=O)c1)C1CC1] | LCMS: 533.1 [M + H]⁺. ¹H NMR (400 MHz, CD₃OD): δ 8.59 (s, 1H), 8.40 (s, 1H), 7.79 (dd, 1H), 7.73-7.70 (m, 3H), 7.66 (dd, 1H), 7.51 (dd, 1H), 7.32 (d, 1H), 5.13 (s, 2H), 4.17 (ABq, 2H), 3.20 (s, 3H), 2.38-2.28 (m, 1H), 1.37 (d, 3H), 0.98-0.88 (m, 1H), 0.78-0.68 (m, 1H), 0.67-0.58 (m, 1H), 0.51-0.42 (m, 1H), 0.28-0.20 (m, 1H). |
| | Starting materials: Int-93 and Int-107. | |
| | Reaction conditions: B′. | |
| | Single enantiomer of unknown | |
| | absolute configuration, separated by | |
| | chiral HPLC. | |
| 109 | 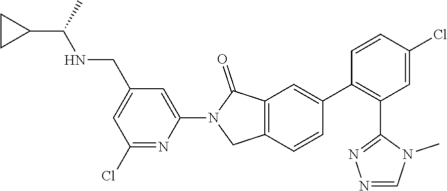 [SMILES: C[C@H](NCc1cc(Cl)nc(N2Cc3ccc(-c4ccc(Cl)cc4-c4nncn4C)cc3C2=O)c1)C1CC1] | LCMS: 533.1 [M + H]⁺. ¹H NMR (400 MHz, CD₃OD): δ 8.55 (d, 1H), 8.40 (s, 1H), 7.79 (dd, 1H), 7.73-7.70 (m, 3H), 7.65 (dd, 1H), 7.49 (dd, 1H), 7.29 (d, 1H), 5.12 (s, 2 H), 4.06 (ABq, 2H), 3.19 (s, 3H), 2.18-2.10 (m, 1H), 1.29 (d, 3H), 0.88-0.80 (m, 1H), 0.69-0.61 (m, 1H), 0.58-0.52 (m, 1H), 0.41-0.33 (m, 1H), 0.19-0.10 (m, 1H). |
| | Starting materials: Int-93 and Int-107. | |
| | Reaction conditions: B′. | |
| | Single enantiomer of unknown | |
| | absolute configuration, separated by | |
| | chiral HPLC. | |
| 110 | 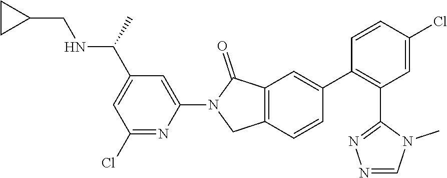 [SMILES: C[C@@H](NCC1CC1)c1cc(Cl)nc(N2Cc3ccc(-c4ccc(Cl)cc4-c4nncn4C)cc3C2=O)c1] | LCMS: 533.2 [M + H]⁺. ¹H NMR (400 MHz, CD₃OD): δ 8.48 (d, 1H), 8.40 (s, 1H), 7.79 (dd, 1H), 7.73-7.70 (m, 3H), 7.64 (dd, 1H), 7.48 (dd, 1H), 7.25 (d, 1H), 5.12 (s, 2H), 3.91 (q, 1H), 3.18 (s, 3H), 2.41 (dd, 1H), 2.27 (dd, 1H), 1.42 (d, 3H), 1.01-0.91 (m, 1H), 0.53-0.48 (m, 2H), 0.18-0.07 (m, 2H). |
| | Starting materials: Int-93 and Int-103. | |
| | Reaction conditions: B′; | |
| | Single enantiomer of unknown | |
| | absolute configuration, separated by | |
| | chiral HPLC. | |
| 111 | 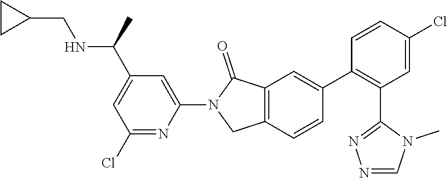 [SMILES: C[C@H](NCC1CC1)c1cc(Cl)nc(N2Cc3ccc(-c4ccc(Cl)cc4-c4nncn4C)cc3C2=O)c1] | LCMS: 533.1 [M + H]⁺. ¹H NMR (400 MHz, CD₃OD): δ 8.48 (s, 1H), 8.40 (s, 1H), 7.79 (dd, 1H), 7.73-7.70 (m, 3H), 7.65 (dd, 1H), 7.48 (dd, 1H), 7.26 (d, 1H), 5.12 (s, 2H), 3.93 (q, 1H), 3.18 (s, 3H), 2.43 (dd, 1H), 2.29 (dd, 1H), 1.43 (d, 3H), 1.01-0.90 (m, 1H), 0.55-0.48 (m, 2H), 0.19-0.10 (m, 2H). |
| | Starting materials: Int-93 and Int-103. | |
| | Reaction conditions: B′ | |
| | Single enantiomer of unknown | |
| | absolute configuration, separated by | |
| | chiral HPLC. | |
| 112 | 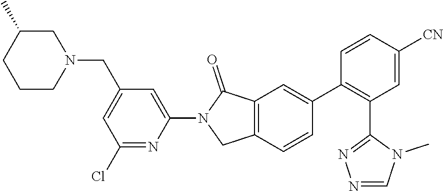 [SMILES: C[C@H]1CCCN(Cc2cc(Cl)nc(N3Cc4ccc(-c5ccc(C#N)cc5-c5nncn5C)cc4C3=O)c2)C1] | LCMS: 538.3 [M + H]⁺. ¹H NMR (400 MHz, DMSO-d6): δ 8.49 (s, 1H), 8.48 (s, 1H), 8.19 (dd, 1H), 8.14 (d, 1H), 7.90 (d, 1H), 7.69 (d, 1H), 7.59 (d, 1H), 7.46 (dd, 1H), 7.21 (s, 1H), 5.08 (s, 2H), 3.53 (s, 2H), 3.17 (s, 3H), 2.78-2.65 (m, 2H), 2.03-1.90 (m, 1H), 1.70-1.58 (m, 4H), 1.57-1.44 (s, 1H), 0.92-0.80 (m, 4H). |
| | Starting materials: Int-96 and Int-18 | |
| | Reaction conditions: B′ | |
| 113 | 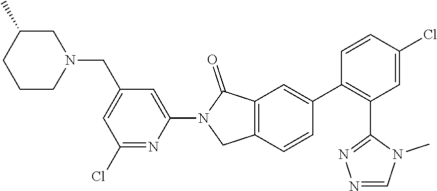 [SMILES: C[C@H]1CCCN(Cc2cc(Cl)nc(N3Cc4ccc(-c5ccc(Cl)cc5-c5nncn5C)cc4C3=O)c2)C1] | LCMS: 547.2 [M + H]⁺. ¹H-NMR (400 MHz, CD₃OD): δ 8.53 (s, 1H), 8.40 (s, 1H), 7.80 (dd, 1H), 7.73-7.70 (m, 3H), 7.64 (d, 1H), 7.48 (dd, 1H), 7.24 (s, 1H), 5.13 (s, 2H), 3.61 (s, 2H), 3.20 (s, 3H), 2.91-2.80 (m, 2H), 2.11-2.01 (m, 1H), 1.80-1.60 (m, 5H), 1.01-0.89 (m, 4H). |
| | Starting materials: Int-93 and Int-18 | |
| | Reaction conditions: A′ | |
| 114 | 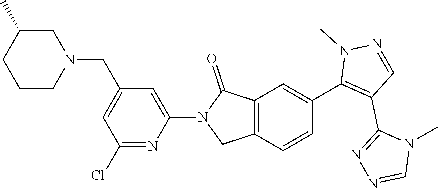 [SMILES: C[C@H]1CCCN(Cc2cc(Cl)nc(N3Cc4ccc(-c5c(-c6nncn6C)cnn5C)cc4C3=O)c2)C1] | LCMS: 517.3 [M + H]⁺. ¹H NMR (400 MHz, DMSO-d6): δ 8.52 (s, 1H), 8.45 (s, 1H), 7.94 (s, 1H) , 7.87 (s, 1H), 7.81 (dd, 1H), 7.78 (dd, 1H), 7.22 (s, 1H), 5.13 (s, 2H), 3.86 (s, 3H), 3.54 (s, 2H), 3.47 (s, 3H), 2.78-2.69 (m, 2H), 2.02- 1.95 (m, 1H), 1.72-1.58 (m, 5H), 0.87- 0.82 (m, 4H). |
| | Starting materials: Int-97 and Int-18. | |
| | Reaction conditions: A′ | |
| 115 | 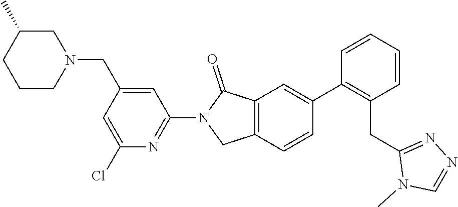 [SMILES: C[C@H]1CCCN(Cc2cc(Cl)nc(N3Cc4ccc(-c5ccccc5Cc5nncn5C)cc4C3=O)c2)C1] | LCMS: 527.40 [M + H]⁺. ¹H NMR (400 MHz, DMSO-d6): δ 8.52 (s, 1H), 8.31 (s, 1H), 7.78 (d, 1H), 7.72 (d, 2H), 7.39-7.37 (m, 2H), 7.33-7.31 (m, 1H), 7.22 (s, 1H), 7.16-7.14 (m, 1H), 5.12 (s, 2H), 4.05 (s, 2H), 3.32 (s, 3H), 2.78-2.70 (m, 2H), 2.05-1.92 (m, 1H), 1.71-1.45 (m, 5H), 0.90-0.80 (m, 4H). |
| | Starting materials: Int-94 and Int-18. | |
| | Reaction conditions: A′ | |
| 116 | 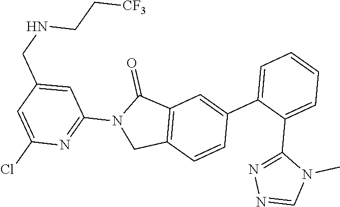 [SMILES: Cn1cnnc1-c1ccccc1-c1ccc2c(c1)C(=O)N(c1cc(CNCCC(F)(F)F)cc(Cl)n1)C2] | LCMS: 527.1 [M + H]⁺. ¹H NMR (400 MHz, DMSO-d₆): δ 8.48 (s, 1H), 8.42 (s, 1 H), 7.76-7.70 (m, 1H), 7.69-7.62 (m, 2H), 7.61-7.58 (m, 2H), 7.54 (d, 1H), 7.45 (dd, 1H), 7.28 (s, 1H), 5.07 (s, 2H), 3.81 (s, 2H), 3.09 (s, 3H), 2.78-2.70 (m, 2H), 2.04-1.94 (m, 2H). |
| | Starting materials: Int-95 and Int-106. | |
| | Reaction conditions: A′ | |
| 117 | 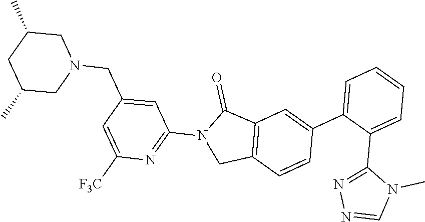 | LCMS: 561.2 [M + H]⁺; ¹H-NMR (400 MHz, DMSO-d6): δ 8.78 (s, 1H), 8.42 (s, 1 H), 7.76-7.67 (m, 3H), 7.65-7.60 (m, 2H), 7.59-7.54 (dd, 2H), 7.45 (dd, 1H), 5.11 (s, 2H), 3.64 (s, 2H), 3.10 (s, 3H), 2.77 (br d, 2H), 1.75-1.61 (m, 3H), 1.55 (t, 2H), 0.81 (d, 6H), 0.60 - 0.48 (m, 1H). |
| | Starting materials: Int-95 and Int-62 | |
| | Reaction conditions: A′ | |
| 118 | 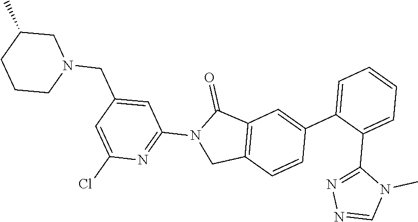 [SMILES: C[C@H]1CCCN(Cc2cc(Cl)nc(N3Cc4ccc(-c5ccccc5-c5nncn5C)cc4C3=O)c2)C1] | LCMS: 513.4 [M + H]+. 1H NMR (400 MHz, DMSO-d6): δ 8.49 (d, 1H), 8.42 (s, 1 H), 7.75-7.71 (m, 1H), 7.69-7.64 (m, 2H), 7.62-7.56 (m, 2H), 7.55 (d, 1H), 7.44 (dd, 1H), 7.21 (s, 1H), 5.07 (s, 2H), 3.53 (s, 2H), 3.09 (s, 3H), 2.69-2.56 (m, 2H), 2.05-1.9 (m, 1H), 1.70-1.59 (m, 4H), 1.55-1.47 (m, 1H), 0.90-0.80 (m, 4H). |
| | Starting materials: Int-95 and Int-18. | |
| | Reaction conditions: A′ | |
| 119 | 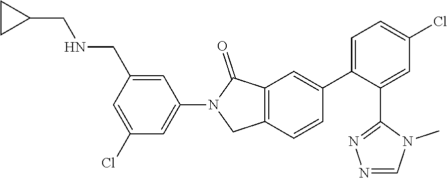 [SMILES: Cn1cnnc1-c1cc(Cl)ccc1-c1ccc2c(c1)C(=O)N(c1cc(Cl)cc(CNCC3CC3)c1)C2] | LCMS: 518.2 [M + H]⁺. ¹H NMR (400 MHz, DMSO-d6): δ 8.44 (s, 1H), 7.96 (dd, 1H), 7.80 (dd, 1H), 7.75 (s, 1H), 7.72-7.70 (m, 2H), 7.60 (d, 1H), 7.53 (dd, 1H), 7.38 (dd, 1H), 7.23 (d, 1H), 5.04 (s, 2H), 3.76 (s, 2H), 3.12 (s, 3H), 2.38 (d, 2H), 0.94-0.85 (m, 1H), 0.46- 0.36 (m, 2H), 0.13-0.06 (m, 2H). |
| | Starting materials: Int-93 and Int-109. | |
| | Reaction conditions: B′ | |
| 120 | 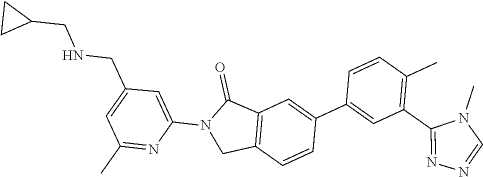 [SMILES: Cc1cc(CNCC2CC2)cc(N2Cc3ccc(-c4ccc(C)c(-c5nncn5C)c4)cc3C2=O)n1] | LCMS: 479.20 [M + H]⁺. ¹H NMR (400 MHz, CD3OD): 8 8.65 (s, 1H), 8.35 (d, 1H), 8.13 (d, 1H), 8.00 (dd, 1H), 7.87 (dd, 1H), 7.77-7.75 (m, 2H), 7.58 (d, 1H), 7.07 (d, 1H), 5.20 (s, 2H), 3.87 (s, 2H), 3.67 (s, 3H), 2.53 (m, 3H), 2.52 (d, 2H), 2.29 (s, 3H), 1.08-0.99 (m, 1H), 0.58-0.52 (m, 2H), 0.23-0.20 (m, 2H). |
| | Starting materials: Int-98 and Int-100. | |
| | Reaction conditions: C′ | |
| 121 | 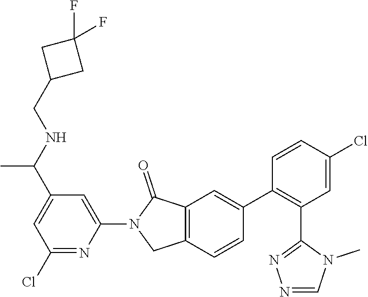 [SMILES: CC(NCC1CC(F)(F)C1)c1cc(Cl)nc(N2Cc3ccc(-c4ccc(Cl)cc4-c4nncn4C)cc3C2=O)c1] | LCMS: 583.3 [M + H]⁺. ¹H NMR (400 MHz, DMSO-d6): δ 8.49 (s, 1H), 8.44 (s, 1H), 7.80 (dd, 1H), 7.73-7.70 (m, 2H), 7.66 (dd, 1H), 7.53 (d, 1H), 7.42 (dd, 1H), 7.30 (s, 1H), 5.06 (s, 2H), 3.80-3.73 (br m, 1H), 3.12 (s, 3H), 2.65-2.50 (m, 3H), 2.40-2.33 (m, 1H), 2.31-2.12 (m, 3H), 1.28 (d, 3H). |
| | Starting materials: Int-93 and Int-114. | |
| | Reaction conditions: C′ | |
| 122 | 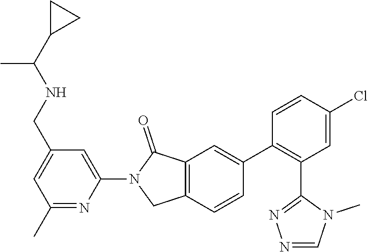 [SMILES: Cc1cc(CNC(C)C2CC2)cc(N2Cc3ccc(-c4ccc(Cl)cc4-c4nncn4C)cc3C2=O)n1] | LCMS: 513.2 [M + H]⁺. ¹H NMR (400 MHz, DMSO-d6): δ 8.44 (s, 1H), 8.31 (s, 1 H), 7.80 (dd, 1H), 7.72-7.70 (m, 2H), 7.65 (d, 1H), 7.52 (d, 1H), 7.39 (dd, 1H), 7.05 (s, 1H), 5.08 (s, 2H), 3.82 (ABq, 2H), 3.12 (s, 3H), 2.45 (s, 3H), 1.94-1.82 (m, 1H), 1.09 (d, 3H), 0.72-0.61 (m, 1H), 0.50-0.41 (m, 1H), 0.38-0.30 (m, 1H), 0.26-0.19 (m, 1H), 0.05-0.01 (m, 1H). |
| | Starting materials: Int-93 and Int-110. | |
| | Reaction conditions: A′ | |
| 123 | 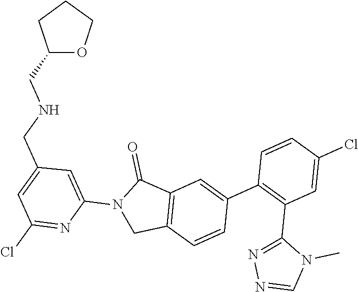 [SMILES: Cn1cnnc1-c1cc(Cl)ccc1-c1ccc2c(c1)C(=O)N(c1cc(CNC[C@@H]3CCCO3)cc(Cl)n1)C2] | LCMS: 549.2 [M + H]⁺. ¹H NMR (400 MHz, DMSO-d₆): δ 8.48 (d, 1H), 8.44 (s, 1 H), 7.80 (dd, 1H), 7.73-7.70 (m, 2H), 7.66 (d, 1H), 7.54 (d, 1H), 7.42 (dd, 1H), 7.27 (d, 1H), 5.07 (s, 2H), 3.95-3.85 (m, 1H), 3.82 (s, 2H), 3.80- 3.70 (m, 1H), 3.65-3.58 (m, 1H), 3.12 (s, 3H), 2.62-2.43 (m, 2H), 1.95-1.88 (m, 1H), 1.86-1.75 (m, 2H), 1.60-1.52 (m, 1H). |
| | Starting materials: Int-93 and Int-111. | |
| | Reaction conditions: C′ | |
| | Single enantiomer of unknown | |
| | absolute configuration, separated by | |
| | chiral HPLC. | |
| 124 | 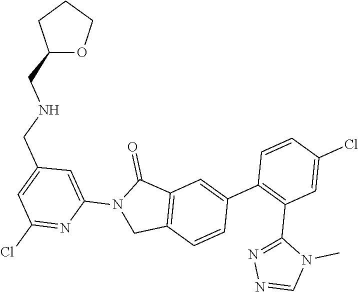 [SMILES: Cn1cnnc1-c1cc(Cl)ccc1-c1ccc2c(c1)C(=O)N(c1cc(CNC[C@H]3CCCO3)cc(Cl)n1)C2] | LCMS: 549.2 [M + H]⁺. ¹H NMR (400 MHz, DMSO-d₆): δ 8.48 (d, 1H), 8.44 (s, 1 H), 7.80 (dd, 1H), 7.73-7.69 (m, 2H), 7.66 (d, 1H), 7.54 (d, 1H), 7.42 (dd, 1H), 7.26 (d, 1H), 5.07 (s, 2H), 3.94-3.85 (m, 1H), 3.81 (s, 2H), 3.80- 3.70 (m, 1H), 3.65-3.58 (m, 1H), 3.12 (s, 3H), 2.60-2.40 (m, 2H), 1.98-1.88 (m, 1H), 1.87-1.76 (m, 2H), 1.61-1.51 (m, 1H). |
| | Starting materials: Int-93 and Int-111. | |
| | Reaction conditions: C′ | |
| | Single enantiomer of unknown | |
| | absolute configuration, separated by | |
| | chiral HPLC. | |
| 125 | 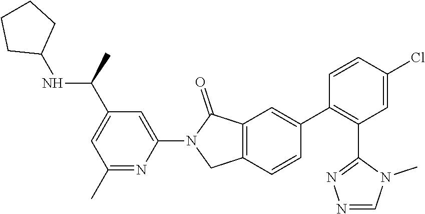 [SMILES: Cc1cc([C@H](C)NC2CCCC2)cc(N2Cc3ccc(-c4ccc(Cl)cc4-c4nncn4C)cc3C2=O)n1] | LCMS: 527.20 [M + H]⁺. ¹H NMR (400 MHz, CD₃OD): δ 8.40 (s, 1H), 8.29 (s, 1H), 7.79 (dd, 1H), 7.73-7.70 (m, 3H), 7.63 (dd, 1H), 7.45 (dd, 1H), 7.08 (s, 1H), 5.16 (s, 2H), 3.86 (q, 1H), 3.18 (s, 3H), 2.92-2.86 (m, 1H), 2.53 (s, 3H), 1.92-1.65 (m, 4H), 1.55-1.44 (m, 2H), 1.40 (d, 3H), 1.39-1.30 (m, 2H). |
| | Starting materials: Int-93 and Int-115. | |
| | Reaction conditions: C′ | |
| | Single enantiomer of unknown | |
| | absolute configuration, separated by | |
| | chiral HPLC. | |
| 126 | 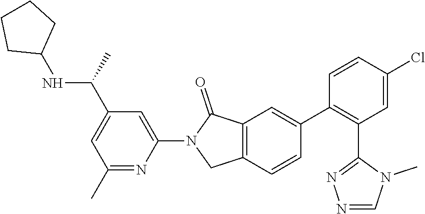 [SMILES: Cc1cc([C@@H](C)NC2CCCC2)cc(N2Cc3ccc(-c4ccc(Cl)cc4-c4nncn4C)cc3C2=O)n1] | LCMS: 527.20 [M + H]⁺. ¹H-NMR (400 MHz, CD₃OD): δ 8.40 (s, 1H), 8.29 (s, 1H), 7.79 (dd, 1H), 7.73-7.70 (m, 3H), 7.63 (dd, 1H), 7.45 (dd, 1H), 7.08 (s, 1H), 5.15 (s, 2H), 3.86 (q, 1H), 3.18 (s, 3H), 2.92-2.86 (m, 1H), 2.53 (s, 3H), 1.92-1.65 (m, 4H), 1.55-1.45 (m, 2H), 1.40 (d, 3H), 1.39-1.29 (m, 2H). |
| | Starting materials: Int-93 and Int-115. | |
| | Reaction conditions: C′ | |
| | Single enantiomer of unknown | |
| | absolute configuration, separated by | |
| | chiral HPLC. | |
| 127 | 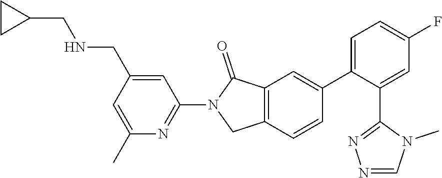 [SMILES: Cc1cc(CNCC2CC2)cc(N2Cc3ccc(-c4ccc(F)cc4-c4nncn4C)cc3C2=O)n1] | LCMS: 483.3 [M + H]⁺. 1H-NMR (400 MHz, DMSO-d6): δ 8.44 (s, 1H), 8.31 (s, 1H), 7.73 (dd, 1H), 7.64 (dd, 1H), 7.58 (ddd, 1H), 7.53-7.50 (m, 2H), 7.38 (dd, 1H), 7.04 (s, 1H), 5.08 (s, 2H), 3.75 (s, 2H), 3.14 (s, 3H), 2.45 (s, 3H), 2.39 (br d, 2H), 0.99-0.86 (m, 1H), 0.45-0.37 (m, 2H), 0.14-0.07 (m, 2H). |
| | Starting materials: Int-99 and Int-100. | |
| | Reaction conditions: B′ | |

### Example 11. 2-(4-((2-Methyl-2,5,6,7-tetrahydro-4H-pyrazolo[4,3-b]pyridin-4-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 128)

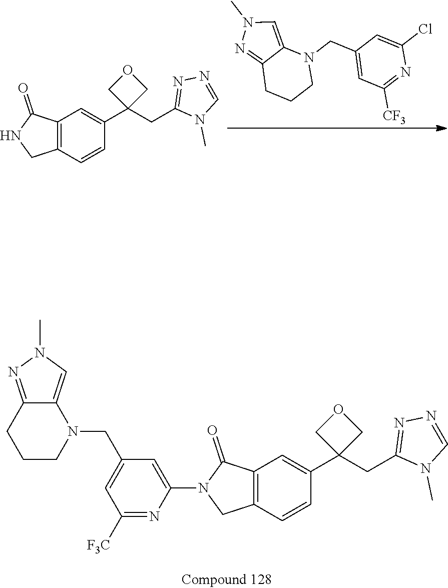 [SMILES: Cn1cnnc1CC1(c2ccc3c(c2)C(=O)NC3)COC1 ; Cn1cc2c(n1)CCCN2Cc1cc(Cl)nc(C(F)(F)F)c1 ; Cn1cc2c(n1)CCCN2Cc1cc(N2Cc3ccc(C4(Cc5nncn5C)COC4)cc3C2=O)nc(C(F)(F)F)c1]

To a degassed solution of 4-((2-chloro-6-(trifluoromethyl)pyridin-4-yl)methyl)-2-methyl-4,5,6,7-tetrahydro-2H-pyrazolo[4,3-b]pyridine (0.1 g, 0.30 mmol), 6-(3-((4-methyl-4H-1,2,4-triazol-3-yl)methyl)oxetan-3-yl)isoindolin-1-one (0.103 g, 0.36 mmol), Cs₂CO₃ (0.25 g, 0.75 mmol) and Xantphos (0.03 g, 0.06 mmol) in DMSO (5.0 ml) was added Pd(OAc)₂ (0.014 g, 0.06 mmol) and heated at 100° C. for 1 h. The mixture was cooled to RT, passed through celite bed followed by washing the celite pad with 10% methanol in DCM. The filtrate was diluted with water and extracted with 10% methanol in DCM. The combined organic layers were washed with brine, dried over anhydrous sodium sulphate, filtered, concentrated, and purified by preparative TLC using 7% methanol in DCM as eluent to afford the title compound (0.035 g, 20%). LCMS: 579.4 [M+H]⁺. ¹HNMR (400 MHz, DMSO-d6): δ 8.81 (s, 1H), 8.61 (bs, 1H), 7.68 (d, 1H), 7.61 (s, 1H), 7.57 (s, 1H), 7.43 (dd, 1H), 6.93 (s, 1H), 5.08 (s, 2H), 4.95 (d, 2H), 4.92 (d, 2H), 4.22 (s, 2H), 3.67 (s, 2H), 3.48 (s, 3H), 3.15 (s, 3H), 2.94-2.90 (m, 2H), 2.60-2.56 (m, 2H), 1.97-1.95 (m, 2H).

### Example 12. 2-(6-Chloro-4-((((2-methylthiazol-4-yl)methyl)amino)methyl)-pyridin-2-yl)-6-(3-((4-methyl-4H-1,2,4-triazol-3-yl)methyl)oxetan-3-yl)isoindolin-1-one (Compound 129)

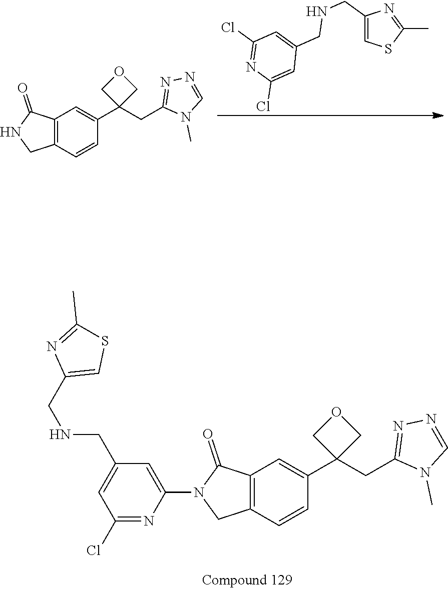 [SMILES: Cn1cnnc1CC1(c2ccc3c(c2)C(=O)NC3)COC1 ; Cc1nc(CNCc2cc(Cl)nc(Cl)c2)cs1 ; Cc1nc(CNCc2cc(Cl)nc(N3Cc4ccc(C5(Cc6nncn6C)COC5)cc4C3=O)c2)cs1]

To a degassed solution of 6-(3-((4-methyl-4H-1,2,4-triazol-3-yl)methyl)oxetan-3-yl)isoindolin-1-one (0.2 g, 0.70 mmol), 1-(2,6-dichloropyridin-4-yl)-N-((2-methyl-thiazol-4-yl)methyl)methanamine (0.24 g, 0.84 mmol), Cs₂CO₃ (0.69 g, 2.10 mmol) and Xantphos (0.041 g, 0.07 mmol) in dioxane (5.0 ml) was added Pd(OAc)₂ (0.016 g, 0.07 mmol) and heated at 90° C. for 2 h. The mixture was cooled to RT, passed through celite bed followed by washing the celite pad with 10% methanol in DCM. The filtrate was diluted with water and extracted with 10% methanol in DCM. The combined organic layer was treated as in Example 11 to afford the title compound (0.006 g, 15%); LCMS: 536.1 [M+H]⁺; ¹H-NMR (400 MHz, DMSO-d6): δ 8.48 (s, 1H), 8.21 (s, 1H), 7.62 (d, 1H), 7.50 (s, 1H), 7.37 (dd, 1H), 7.28 (d, 1H), 7.27 (s, 1H), 5.03 (s, 2H), 4.97 (d, 2H), 4.91 (d, 2H), 3.84 (s, 2H), 3.76 (s, 2H), 3.57 (s, 2H), 3.02 (s, 3H), 2.63 (s, 3H).

### Example 13: 2-(6-Chloro-4-((((1-fluorocyclopropyl)methyl)amino)methyl)-pyridin-2-yl)-6-(3-((4-methyl-4H-1,2,4-triazol-3-yl)methyl)oxetan-3-yl)isoindolin-1-one (Compound 130)

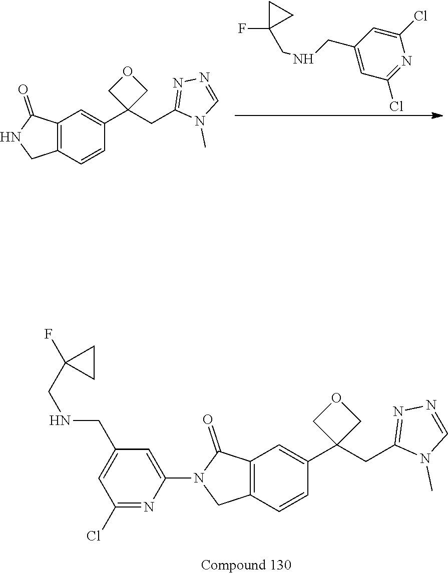 [SMILES: Cn1cnnc1CC1(c2ccc3c(c2)C(=O)NC3)COC1 ; FC1(CNCc2cc(Cl)nc(Cl)c2)CC1 ; Cn1cnnc1CC1(c2ccc3c(c2)C(=O)N(c2cc(CNCC4(F)CC4)cc(Cl)n2)C3)COC1]

To a degassed solution of 6-(3-((4-methyl-4H-1,2,4-triazol-3-yl)methyl)oxetan-3-yl)isoindolin-1-one (0.15 g, 0.53 mmol), 1-(2,6-dichloropyridin-4-yl)-N-((1-fluoro-cyclopropyl)methyl)methanamine (0.16 g, 0.63 mmol), Cs₂CO₃ (0.34 g, 1.05 mmol) and Xantphos (0.06 g, 0.10 mmol) in dioxane (5.0 ml) was added Pd₂(dba)₃ (0.05 g, 0.05 mmol) and heated at 90° C. for 2 h. The mixture was cooled to RT, passed through celite bed followed by washing the celite pad with 10% methanol in DCM. The filtrate was diluted with water and extracted with 10% methanol in DCM. The combined organic layer was treated as in Example 11 using 5% methanol in DCM as eluent to afford the title compound (0.02 g, 7.62%). LCMS: 497.3 [M+H]⁺. ¹HNMR (400 MHz, DMSO-d6): δ 8.49 (s, 1H), 8.21 (s, 1H), 7.62 (dd, 1H), 7.51 (s, 1H), 7.36 (dd, 1H), 7.28 (s, 1H), 5.05 (s, 2H), 4.97 (d, 2H), 4.92 (d, 2H), 3.89 (s, 2H), 3.57 (s, 2H), 3.03 (s, 3H), 2.90 (d, 2H), 2.65-2.58 (m, 1H), 0.90-1.01 (m, 2H), 0.71-0.65 (m, 2H).

### Example 14. 2-(6-Chloro-4-(((1-cyclopropylethyl)amino)methyl)pyridin-2-yl)-6-(3-((4-methyl-4H-1,2,4-triazol-3-yl)methyl)oxetan-3-yl)isoindolin-1-one (Compound 131)

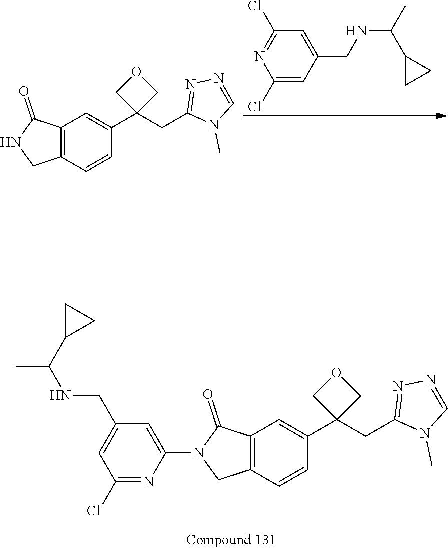

To a degassed solution of 6-(3-((4-methyl-4H-1,2,4-triazol-3-yl)methyl)oxetan-3-yl)isoindolin-1-one (0.20 g, 0.70 mmol), 1-cyclopropyl-N-((2,6-dichloropyridin-4-yl)methyl)ethan-1-amine (0.224 g, 0.91 mmol), Cs₂CO₃ (0.46 g, 1.40 mmol) and DavePhos (0.055 g, 0.14 mmol) in DMSO (2.0 ml) was added Pd(OAc)₂ (0.016 g, 0.07 mmol) and heated at 90° C. for 2 h. The mixture was cooled to RT, passed through celite bed followed by washing the celite pad with 10% methanol in DCM. The filtrate was diluted with water and extracted with 10% methanol in DCM. The combined organic layer was treated as in Example 11 using 5% methanol in DCM as eluent to afford the title compound (0.075 g, 22%). LCMS: 493.15 [M+H]⁺. ¹H-NMR (400 MHz, DMSO-d6): δ 8.50 (s, 1H), 8.21 (s, 1H), 7.62 (d, 1H), 7.51 (s, 1H), 7.36 (dd, 1H), 7.29 (d, 1H), 5.02 (s, 2H), 4.97 (d, 2H), 4.91 (d, 2H), 3.88 (s, 2H), 3.57 (s, 2H), 3.03 (s, 3H), 1.92-1.85 (m, 1H), 1.09 (d, 3H), 0.70-0.65 (m, 1H), 0.49-0.42 (m, 1H), 0.40-0.30 (m, 1H), 0.29-0.19 (m, 1H).

### Example 15. 6-(4-Chloro-2-(4-methyl-4H-1,2,4-triazol-3-yl)phenyl)-2-(4-(((cyclopropylmethyl)amino)methyl)-6-(trifluoromethyl)pyridin-2-yl)isoindolin-1-one (Compound 132)

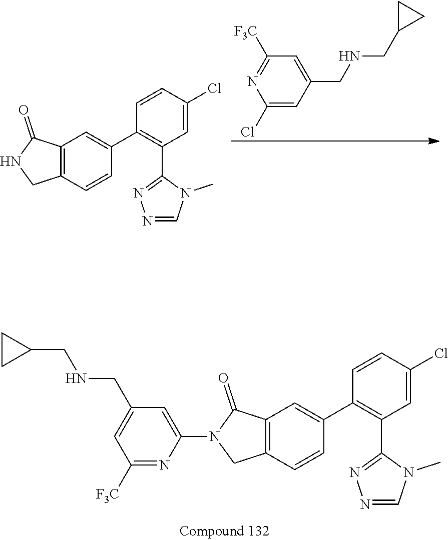

To a degassed solution of 1-(2-chloro-6-(trifluoromethyl)pyridin-4-yl)-N-(cyclo-propylmethyl)methanamine (0.21 g, 0.80 mmol), 6-(4-chloro-2-(4-methyl-4H-1,2,4-triazol-3-yl)phenyl)isoindolin-1-one (0.20 g, 0.62 mmol), Cs₂CO₃ (0.50 g, 1.54 mmol) and Xantphos (0.07 g, 0.12 mmol) in DMSO (2.0 ml) was added Pd(OAc)₂ (0.014 g, 0.06 mmol) and heated at 80° C. for 1 h. The mixture was cooled to RT, passed through celite bed followed by washing the celite pad with 10% methanol in DCM. The filtrate was diluted with water and extracted with 9% methanol in DCM. The combined organic layer was treated as in Example 11 to afford the title compound (0.053 g, 16%). LC-MS: 553.2 [M+H]⁺. ¹H-NMR (400 MHz, DMSO-d6): δ 8.78 (s, 1H), 8.45 (s, 1H), 7.8 (dd, 1H), 7.26-7.66 (m, 4H), 7.55 (d, 1H), 7.43 (dd, 1H), 5.11 (s, 2H), 3.90 (s, 2H), 3.13 (s, 3H), 2.41 (s, 2H), 0.92-0.87 (m, 1H), 0.44-0.39 (m, 2H), 0.14-0.10 (m, 2H).

### Example 16. 2-(6-(((Cyclopropylmethyl)amino)methyl)-2-methylpyrimidin-4-yl)-6-(4-fluoro-2-(4-methyl-4H-1,2,4-triazol-3-yl)phenyl)isoindolin-1-one (Compound 133)

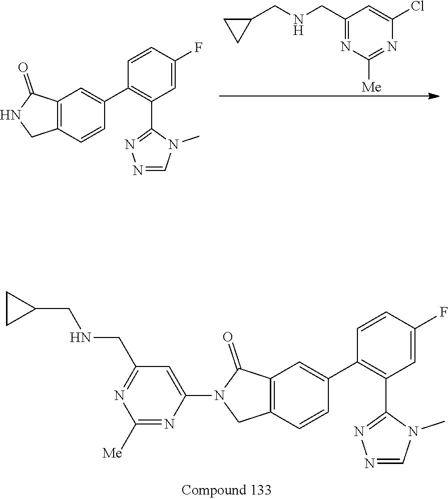

To a degassed solution of 6-(4-fluoro-2-(4-methyl-4H-1,2,4-triazol-3-yl)phenyl)-isoindolin-1-one (0.15 g, 0.49 mmol), 1-(6-chloro-2-methylpyrimidin-4-yl)-N-(cyclo-propylmethyl)methanamine (0.13 g, 0.63 mmol), Cs₂CO₃ (0.48 g, 1.46 mmol) and Xantphos (0.014 g, 0.02 mmol) in dioxane (3.0 ml) was added Pd(OAc)₂ (0.003 g, 0.01 mmol) and heated at 65° C. for 4 h. The mixture was cooled to RT, passed through celite bed followed by washing the celite pad with 10% methanol in DCM. The filtrate was diluted with water and extracted with 10% methanol in DCM. The combined organic layer was treated as in Example 11 using 8% methanol in DCM as eluent to afford the title compound (0.06 g, 25%). LC-MS: 484.30 [M+H]⁺. ¹H-NMR (400 MHz, DMSO-d6): δ 8.45 (s, 1H), 8.41 (s, 1H), 7.75-7.71 (m, 1H), 7.69 (d, 1H), 7.61-7.50 (m, 3H), 7.43 (dd, 1H), 5.07 (s, 2H), 3.79 (s, 2H), 3.16 (s, 3H), 2.67 (s, 3H), 2.44 (d, 2H), 0.95-0.90 (m, 1H), 0.44-0.40 (m, 2H), 0.15-0.13 (m, 2H).

### Example 17. 2-(4-(((Cyclobutylmethyl)amino)methyl)-6-methylpyridin-2-yl)-6-(4-fluoro-2-(4-methyl-4H-1,2,4-triazol-3-yl)phenyl)isoindolin-1-one (Compound 134)

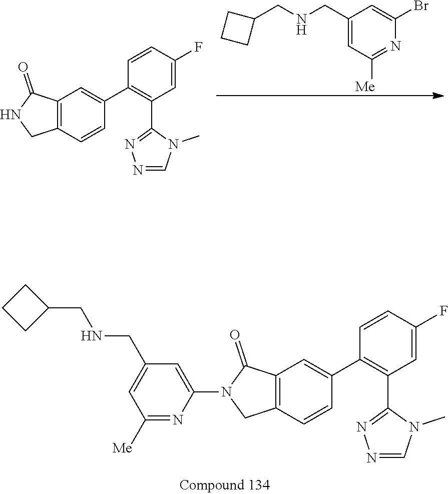

To a degassed solution of 6-(4-fluoro-2-(4-methyl-4H-1,2,4-triazol-3-yl)-phenyl)isoindolin-1-one (0.15 g, 0.49 mmol), 1-(2-bromo-6-methylpyridin-4-yl)-N-(cyclobutylmethyl)methanamine (0.17 g, 0.63 mmol), Cs₂CO₃ (0.48 g, 1.46 mmol) and Xantphos (0.014 g, 0.02 mmol) in dioxane (5.0 ml) was added Pd₂(dba)₃ (0.022 g, 0.02 mmol) and heated at 60° C. for 6 h. The mixture was cooled to RT, passed through celite bed followed by washing the celite pad with 10% methanol in DCM. The filtrate was diluted with water and extracted with 10% methanol in DCM. The combined organic layer was treated as in Example 11 using 5% methanol in DCM as eluent to afford the title compound (0.08 g, 33%). LCMS: 497.2 [M+H]⁺. ¹H-NMR (400 MHz, DMSO-d6): δ 8.44 (s, 1H), 8.30 (s, 1H), 7.72 (dd, 1H), 7.64 (d, 1H), 7.62-7.55 (m, 1H), 7.54-7.48 (m, 2H), 7.38 (dd, 1H), 7.03 (s, 1H), 5.08 (s, 2H), 3.71 (s, 2H), 3.13 (s, 3H), 2.54-2.51 (m, 2H), 2.45 (s, 3H), 2.45-2.38 (m, 1H), 2.05-1.95 (m, 2H), 1.90-1.78 (m, 2H), 1.73-1.59 (m, 2H).

### Example 18. 6-(4-Chloro-2-(4-methyl-4H-1,2,4-triazol-3-yl)phenyl)-2-(4-(1-((cyclopropylmethyl)amino)ethyl)-6-methylpyridin-2-yl)isoindolin-1-one (Compound 135)

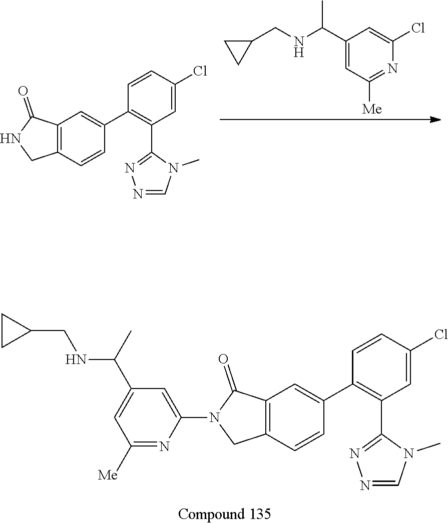

To a degassed solution of 6-(4-chloro-2-(4-methyl-4H-1,2,4-triazol-3-yl)-phenyl)isoindolin-1-one (0.30 g, 0.92 mmol), 1-(2-chloro-6-methylpyridin-4-yl)-N-(cyclopropylmethyl)ethan-1-amine (0.31 g, 1.34 mmol), Cs₂CO₃ (0.90 g, 2.77 mmol) and DavePhos (0.073 g, 0.18 mmol) in DMSO (6.0 ml) was added Pd(OAc)₂ (0.021 g, 0.09 mmol) and heated at 100° C. for 2 h. The mixture was cooled to RT, passed through celite bed followed by washing the celite pad with 10% methanol in DCM. The filtrate was diluted with water and extracted with 10% methanol in DCM. The combined organic layer was treated as in Example 11 using 5% methanol in DCM as eluent to afford the title compound (0.05 g, 12%). LCMS: 513.2 [M+H]⁺. ¹H-NMR (400 MHz, DMSO-d6): δ 8.44 (s, 1H), 8.30 (s, 1H), 7.80 (dd, 1H), 7.72-7.70 (m, 2H), 7.66 (d, 1H), 7.50 (d, 1H), 7.40 (dd, 1H), 7.05 (s, 1H), 5.08 (s, 2H), 3.75 (q, 1H), 3.11 (s, 3H), 2.45 (s, 3H), 2.28-2.18 (m, 2H), 1.26 (d, 3H), 0.88-0.80 (m, 1H), 0.40-0.32 (m, 2H), 0.08-0.02 (m, 2H).

### Example 19. 6-(4-Fluoro-2-(4-methyl-4H-1,2,4-triazol-3-yl)phenyl)-2-(4-(hydroxymethyl)-6-methylpyridin-2-yl)isoindolin-1-one (Compound 136)

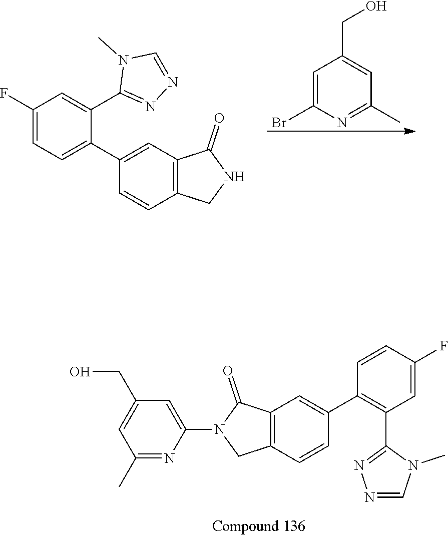

To a degassed solution of 6-(4-fluoro-2-(4-methyl-4H-1,2,4-triazol-3-yl) phenyl) isoindolin-1-one (3.00 g, 9.73 mmol), (2-bromo-6-methylpyridin-4-yl) methanol (2.36 g, 11.68 mmol), and Cs₂CO₃ (9.51 g, 29.19 mmol) in 1,4-dioxane (60.0 mL) was added Xantphos-Pd-G3 (0.93 g, 0.97 mmol) and stirred at 100° C. for 6 h. The mixture was cooled to RT, filtered through celite pad followed by washing with 10% MeOH in DCM. The filtrate was washed with brine, dried over anhydrous sodium sulphate, concentrated, and purified by silica-gel flash column chromatography using 6% MeOH in DCM as eluent to afford the title compound as an off-white solid (3.00 g, 72%). LCMS: 430.1 [M+H]⁺. ¹H-NMR (400 MHz, DMSO-d6): δ 8.45 (s, 1H), 8.32 (s, 1H), 7.73 (dd, 1H), 7.64 (d, 1H), 7.58 (dt, 1H), 7.55-7.50 (m, 2H), 7.38 (dd, 1H), 7.00 (s, 1H), 5.46 (t, 1H), 5.09 (s, 2H), 4.55 (d, 2H), 3.15 (s, 3H), 2.46 (s, 3H).

### Example 20: 6-(4-Fluoro-2-(4-methyl-4H-1,2,4-triazol-3-yl)phenyl)-2-(4-((((1-methoxycyclopropyl)methyl)amino)methyl)-6-methylpyridin-2-yl)isoindolin-1-one (Compound 137)

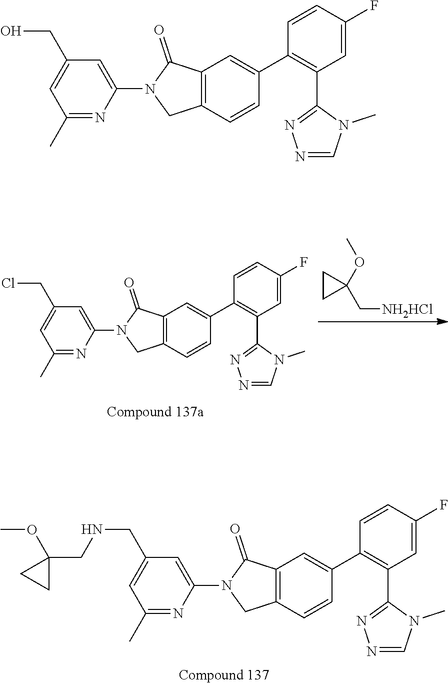

### a) 2-(4-(Chloromethyl)-6-methylpyridin-2-yl)-6-(4-fluoro-2-(4-methyl-4H-1,2,4-triazol-3-yl) phenyl) isoindolin-1-one (Compound 137a)

To a solution of 6-(4-fluoro-2-(4-methyl-4H-1,2,4-triazol-3-yl) phenyl)-2-(4-(hydroxymethyl)-6-methylpyridin-2-yl) isoindolin-1-one (2.40 g, 5.58 mmol) in DCM (20.0 mL) was added thionyl chloride (1.60 mL, 21.80 mmol) 0° C. and then stirred at RT for 16 h. The mixture was concentrated, diluted with saturated sodium bicarbonate solution and extracted with DCM. The combined organic layer was dried over anhydrous sodium sulphate, filtered, concentrated, and purified by silica-gel flash column chromatography using 4% MeOH in DCM as eluent to afford the title compound (2.1 g, 83.91%). LCMS: 448.1 [M+H]⁺.

### b) 6-(4-Fluoro-2-(4-methyl-4H-1,2,4-triazol-3-yl)phenyl)-2-(4-((((1-methoxy-cyclopropyl)methyl)amino)methyl)-6-methylpyridin-2-yl)isoindolin-1-one (Compound 137)

To a solution of compound 137a (0.070 g, 0.15 mmol) and (1-methoxycyclo-propyl)methanamine hydrochloride (0.032 g, 0.234 mmol) in acetonitrile was added K₂CO₃ (0.108 g, 0.78 mmol) and the mixture was stirred at RT for 12 h. The mixture was diluted with water and extracted with 10% methanol in DCM. The combined organic layer was treated as in Example 11 using 5% MeOH in DCM as eluent to afford title compound as off white solid, (0.020 g, 25.01%). LCMS: 513.2 [M+H]⁺. ¹H NMR (400 MHz, DMSO-d6): δ 8.44 (s, 1H), 8.33 (s, 1H), 7.73 (dd, 1H), 7.64 (d, 1H), 7.58 (td, 1H), 7.55-7.48 (m, 2H), 7.38 (dd, 1H), 7.05 (s, 1H), 5.08 (s, 2H), 3.81 (s, 2H), 3.22 (s, 3H), 3.14 (s, 3H), 2.68 (s, 2H), 2.46 (s, 3H), 0.68-0.64 (m, 2H), 0.52-0.48 (m, 2H).

The following compounds were prepared according to the procedure described for Example 11 and 15 (=conditions A″), Example 12 and 16 (=conditions B″), Example 13 and 17 (=conditions C″), Example 14 and 18 (=conditions D″) and Example 20 (=conditions E) with appropriate variations in reactants, quantities of reagents, coupling methods and solvents. The characterization data of the compounds are summarized in the Table.

| | Structure, starting material and | |
|---|---|---|
| No. | reaction conditions | Characterization data |
| 138 | 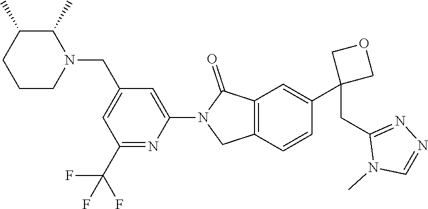 [SMILES: C[C@H]1CCCN(Cc2cc(N3Cc4ccc(C5(Cc6nncn6C)COC5)cc4C3=O)nc(C(F)(F)F)c2)[C@H]1C] Starting materials: Int-1 and Int-169 Reaction conditions: A″ | LCMS: 555.3 [M + H]⁺. ¹HNMR (400 MHz, DMSO-d₆): δ 8.86 (s, 1H), 8.21 (s, 1H), 7.65 (d, 1H), 7.59-7.54 (m, 2H), 7.37 (dd, 1H), 5.07 (s, 2H), 4.97 (d, 2H), 4.91 (d, 2H), 3.75 (ABq, 2H), 3.57 (s, 2H), 3.30-3.29 (m, 1H), 3.03 (s, 3H), 2.78-2.76 (m, 1H), 2.30-2.27 (m, 1H), 1.91-1.81(m, 1H), 1.57-1.42 (m, 3H), 1.30-1.24 (m, 1H), 0.90 (d, 3H), 0.86 (d, 3H). |
| 139 | 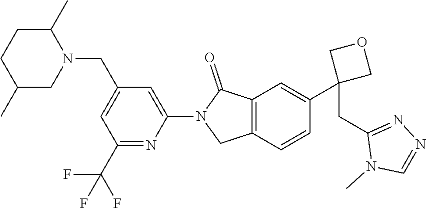 [SMILES: CC1CCC(C)N(Cc2cc(N3Cc4ccc(C5(Cc6nncn6C)COC5)cc4C3=O)nc(C(F)(F)F)c2)C1] Enantiomer 1 of diastereomer 1 Starting materials: Int-1 and Int-170 Reaction conditions: A″ | LCMS: 555.3 [M + H]⁺. ¹HNMR (400 MHz, DMSO-d₆): δ 8.82 (s, 1H), 8.22 (s,1H), 7.66 (d, 1H), 7.58 (s, 1H), 7.53 (d, 1H), 7.38 (dd, 1H), 5.07 (s, 2H), 4.98 (d, 2H), 4.91 (d, 2H), 4.13 (d, 1H), 3.58 (s, 2H), 3.03 (s, 3H), 2.38- 2.19 (m, 1H), 1.70-1.56 (m, 3H), 1.35- 1.28 (m, 2H), 1.08 (d, 3H), 0.99-0.91 (m, 1H), 0.77 (d, 3H). |
| 140 | 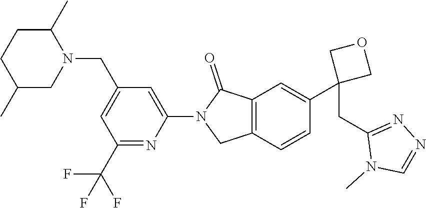 [SMILES: CC1CCC(C)N(Cc2cc(N3Cc4ccc(C5(Cc6nncn6C)COC5)cc4C3=O)nc(C(F)(F)F)c2)C1] Enantiomer 2 of diastereomer 2 Starting materials: Int-1 and Int-170 Reaction conditions: A″ | LCMS: 555.4 [M + H]⁺; ¹HNMR (400 MHz, DMSO-d₆): δ 8.84 (s, 1H), 8.22 (s,1H), 7.65 (d, 1H), 7.60 (s, 1H), 7.54 (d, 1H), 7.37 (dd, 1H), 5.06 (s, 2H), 4.97 (d, 2H), 4.91 (d, 2H), 3.80 (d, 1H), 3.66 (d, 1H), 3.58 (s, 2H), 3.03 (s, 3H), 2.82-2.80 (m, 1H), 2.33-2.09 (m, 2H), 1.69-1.68 (m, 2H), 1.51-1.49 (m, 2H), 1.02 (d, 3H), 0.89 (d, 3H), 0.88- 0.82 (m, 1H). |
| 141 | 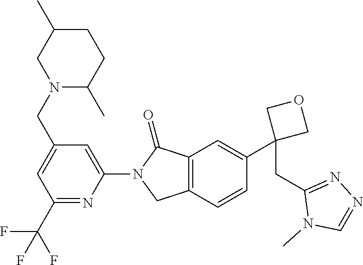 [SMILES: CC1CCC(C)N(Cc2cc(N3Cc4ccc(C5(Cc6nncn6C)COC5)cc4C3=O)nc(C(F)(F)F)c2)C1] Enantiomer 2 of diastereomer 1 Starting materials: Int-1 and Int-170 Reaction conditions: A″ | LCMS: 555.6 [M + H]⁺. ¹HNMR (400 MHz, DMSO-d₆): δ 8.82 (s, 1H), 8.22 (s,1H), 7.66 (d, 1H), 7.58 (s, 1H), 7.53 (d, 1H), 7.38 (dd, 1H), 5.07 (s, 2H), 4.98 (d, 2H), 4.91 (d, 2H), 4.13(d, 1H), 3.57 (s, 2H), 3.03 (s, 3H), 2.68-2.66 (m, 1H), 2.29-2.16 (m, 1H), 1.71-1.64 (m, 2H), 1.60-1.29 (m, 2H), 1.09 (d, 3H), 0.98-0.90 (m, 1H), 0.77 (d, 3H). |
| 142 | 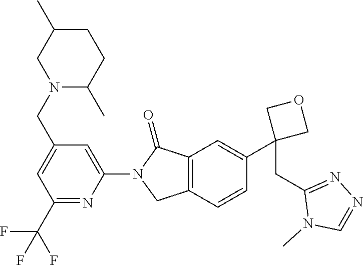 [SMILES: CC1CCC(C)N(Cc2cc(N3Cc4ccc(C5(Cc6nncn6C)COC5)cc4C3=O)nc(C(F)(F)F)c2)C1] Enantiomer 1 of diastereomer 2 Starting materials: Int-1 and Int-170 Reaction conditions: A″ | LCMS: 555.4 [M + H]⁺; ¹HNMR (400 MHz, DMSO-d₆): δ 8.83 (s, 1H), 8.22 (s,1H), 7.65 (d, 1H), 7.60 (s, 1H), 7.54 (d, 1H), 7.37 (dd, 1H), 5.06 (s, 2H), 4.97 (d, 2H), 4.91 (d, 2H), 3.80 (d, 1H), 3.66 (d, 1H), 3.58 (s, 2H), 3.02 (s, 3H), 2.82-2.80 (m, 1H), 2.30-2.29 (m, 1H), 2.26-2.24 (m, 1H), 1.70-1.67 (m, 2H), 1.53-1.48 (m, 2H), 1.02 (d, 3H), 0.89 (d, 3H), 0.88-0.8 (m, 1H). |
| 143 | 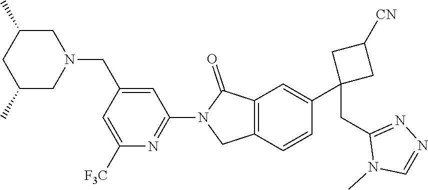 [SMILES: C[C@@H]1C[C@H](C)CN(Cc2cc(N3Cc4ccc(C5(Cc6nncn6C)CC(C#N)C5)cc4C3=O)nc(C(F)(F)F)c2)C1] Starting materials: Int-23 and Int-62 Reaction conditions: A″ | LCMS: 578.0 [M + H]⁺. ¹H-NMR (400 MHz, CDCl₃) 8: 8.77 (s, 1H), 7.89(s, 1 H), 7.75 (d, 1H), 7.54 (s, 1H), 7.46 (d, 1H), 7.22 (dd, 1H), 5.16 (s, 2H), 3.61 (s, 2H), 3.50-3.40 (m, 1H), 3.39 (s, 2H), 3.30-3.21 (m, 1H), 3.20-3.02 (m, 4H), 2.87 (s, 3H), 2.83-2.74 (m, 2H), 1.88-1.70 (m, 3H), 0.86 (d, 6H), 0.61- 0.52 (m, 1H). |
| 144 | 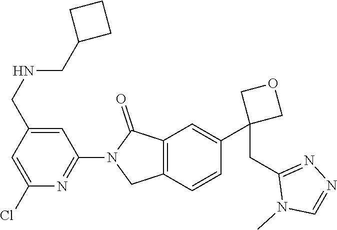 [SMILES: Cn1cnnc1CC1(c2ccc3c(c2)C(=O)N(c2cc(CNCC4CCC4)cc(Cl)n2)C3)COC1] Starting materials: Int-1 and Int-172 Reaction conditions: A″ | LCMS: 495.1 [M + 2 + H]⁺. ¹H-NMR (400 MHz, DMSO-d₆): δ 8.48 (s, 1H), 8.21 (s, 1H), 7.62 (d, 1H), 7.51 (d, 1H), 7.36 (dd, 1H), 7.25 (s, 1H), 5.03 (s, 2H), 4.97 (d, 2H), 4.91 (d, 2H ), 3.77 (s, 2H), 3.57 (s, 2H), 3.03 (s, 3H), 2.53 (s, 2H), 2.45-2.35 (m, 1H), 2.06-1.96 (m, 2H), 1.88-1.75 (m, 2H), 1.72-1.62 (m, 2H). |
| 145 | 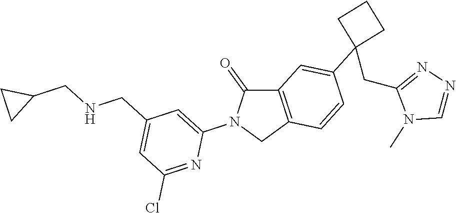 [SMILES: Cn1cnnc1CC1(c2ccc3c(c2)C(=O)N(c2cc(CNCC4CC4)cc(Cl)n2)C3)CCC1] Starting materials: Int-3 and Int-76 Reaction conditions: A″ | LCMS: 477.2 [M + H]⁺. ¹H-NMR (400 MHz, DMSO): δ 8.48 (d, 1H), 8.15 (s, 1H), 7.57 (d, 1H), 7.32 (d, 1H), 7.26 (s, 1H), 7.23 (dd, 1H), 5.01 (s, 2H), 3.80 (s, 2H), 3.30 (s, 2H), 2.77 (s, 3H), 2.63-2.55 (m, 2H), 2.39-2.31 (m, 5H), 2.23-2.15 (m, 1H), 0.95-0.85 (m, 1H), 0.42-0.38 (m, 2H), 0.13-0.09 (m, 2H). |
| 146 | 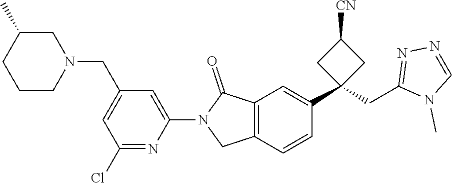 [SMILES: C[C@H]1CCCN(Cc2cc(Cl)nc(N3Cc4ccc([C@]5(Cc6nncn6C)C[C@H](C#N)C5)cc4C3=O)c2)C1] Single enantiomer Starting materials: Int-23 and Int-18 Reaction conditions: A″ | LCMS: 530.3 [M + H]⁺. ¹H-NMR (400 MHz, DMSO-d₆): δ 8.49 (s, 1H), 8.19 (s, 1H), 7.61 (d, 1H), 7.51 (d, 1H), 7.39 (dd, 1H), 7.19 (s, 1H), 5.03 (s, 2H), 3.52 (s, 2H), 3.36 (s, 2H), 3.30- 3.20 (m, 1H), 2.95-2.89 (m, 4H), 2.87 (s, 3H), 2.77-2.65 (m, 2H), 2.0-1.90 (m, 1H), 1.71-1.58 (m, 4H), 1.57-1.45 (m, 1H), 0.92-0.84 (m, 1H). 0.84 (d, 3H). |
| 147 | 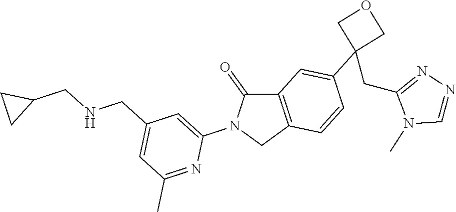 [SMILES: Cc1cc(CNCC2CC2)cc(N2Cc3ccc(C4(Cc5nncn5C)COC4)cc3C2=O)n1] Starting materials: Int-1 and Int-174 Reaction conditions: A″ | LCMS: 459.2 [M + H]⁺. ¹H-NMR (400 MHz, DMSO): δ 8.31 (s, 1H), 8.21 (s, 1H), 7.61 (d, 1H), 7.42 (d, 1H), 7.33 (dd, 1H), 7.03 (s, 1H), 5.05 (s, 2H), 4.98 (d, 2H), 4.91 (d, 2H), 3.75 (s, 2H), 3.57 (s, 2H), 3.01 (s, 3H), 2.45 (s, 3H), 2.38 (d, 2H), 0.94-0.85 (m, 1H), 0.43- 0.39 (m, 2H), 0.13-0.10 (m, 2H). |
| 148 | 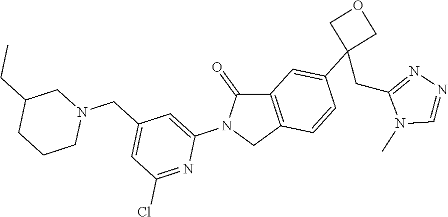 [SMILES: CCC1CCCN(Cc2cc(Cl)nc(N3Cc4ccc(C5(Cc6nncn6C)COC5)cc4C3=O)c2)C1] Starting materials: Int-1 and Int-175 Reaction conditions: A″ | LCMS: 521.3 [M + H]⁺. ¹H-NMR (400 MHz, DMSO-d6): δ 8.49 (s, 1H), 8.21 (s, 1H), 7.61 (d, 1H), 7.51 (d, 1H), 7.41 (d, 1H), 7.12(s, 1H), 5.03 (s, 2H), 4.98 (dd, 2H), 4.91 (dd, 2H), 3.57-3.52 (m, 4H), 3.02 (s, 3H), 2.01-1.96 (m, 2H), 1.79-1.37 (m, 5H), 1.22-1.17 (m, 2H), 0.86-0.83 (m, 4H). |
| 149 | 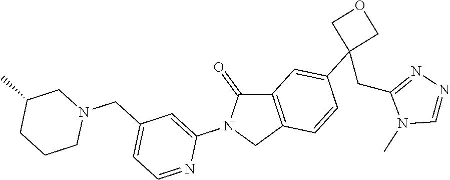 [SMILES: C[C@H]1CCCN(Cc2ccnc(N3Cc4ccc(C5(Cc6nncn6C)COC5)cc4C3=O)c2)C1] Starting materials: Int-1 and Int-176 Reaction conditions: A″ | LCMS: 473.26 [M + H]⁺. ¹H-NMR (400 MHz, DMSO): δ 8.50 (s, 1H), 8.35 (d, 1H), 8.21 (s, 1H), 7.61 (d, 1H), 7.49 (s, 1H), 7.33 (dd, 1H), 7.13 (d, 1H), 5.06 (s, 2H), 4.98 (d, 2H), 4.91 (d, 2H), 3.57 (s, 2H), 3.51 (s, 2H), 3.00 (s, 3H), 2.72-2.67 (m, 2H), 1.99-1.92 (m, 1H), 1.68-1.59 (m, 4H), 1.56-1.45 (m, 1H), 0.92-0.82 (m, 4H). |
| 150 | 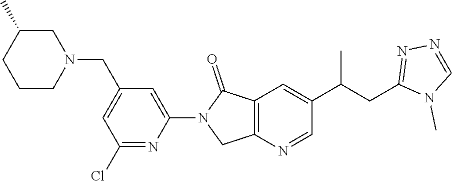 [SMILES: CC(Cc1nncn1C)c1cnc2c(c1)C(=O)N(c1cc(CN3CCC[C@H](C)C3)cc(Cl)n1)C2] Starting materials: Int-249 and Int-18 Reaction conditions: A″ | LCMS: 480.20 [M + H]⁺. ¹H-NMR (400 MHz, DMSO-d₆): δ 8.79 (d, 1H), 8.50 (s, 1H), 8.29 (s, 1H), 8.21 (d, 1H), 7.22 (s, 1H), 5.02 (s, 2H), 3.55 (s, 3H), 3.54 (s, 2H), 3.54-3.49 (m, 1H) 3.13 (d, 2H), 2.74-2.68 (m, 2H), 2.0- 1.92 (m, 1H), 1.72-1.59 (m, 4H), 1.58- 1.48 (m, 1H), 1.38 (d, 3H), (0.95-0.85 (m, 1H), 0.84 (d, 3H). |
| 151 | 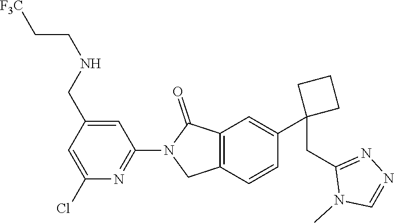 [SMILES: Cn1cnnc1CC1(c2ccc3c(c2)C(=O)N(c2cc(CNCCC(F)(F)F)cc(Cl)n2)C3)CCC1] Starting materials: Int-3 and Int-106 Reaction conditions: A″ | LCMS: 521.1 [M + 2 + H]⁺. ¹H-NMR (400 MHz, DMSO-d₆): δ 8.47 (s, 1H), 8.15 (s, 1H), 7.57 (d, 1H), 7.32 (d, 1H), 7.26 (d, 1H), 7.23 (dd, 1H), 5.02 (s, 2H), 3.81 (d, 2H), 3.25 (s, 2H), 2.77 (d, 3H), 2.72-2.68 (m, 2H), 2.62-2.55 (m, 5H), 2.46-2.45 (m, 1H), 2.38-2.36 (m, 1H), 2.23-2.16 (m, 1H). |
| 152 | 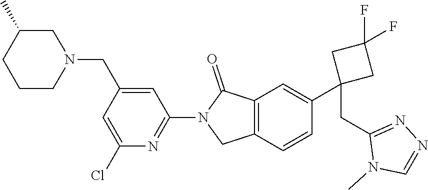 [SMILES: C[C@H]1CCCN(Cc2cc(Cl)nc(N3Cc4ccc(C5(Cc6nncn6C)CC(F)(F)C5)cc4C3=O)c2)C1] Starting materials: Int-18 and Int-124 Reaction conditions: A″ | LCMS: 541.2 [M + H]⁺. ¹H-NMR (400 MHz, DMSO): δ 8.48 (d, 1H), 8.20 (s, 1H), 7.61(d, 1H), 7.50 (d, 1H), 7.36 (dd, 1H), 7.27 (d, 1H), 5.03 (s, 2H), 3.81 (s, 2H), 3.35-3.24 (m, 4H), 3.11- 3.00 (m, 2H), 2.80 (s, 3H), 2.78-2.67 (m, 2H), 2.01-1.90 (m, 1H), 1.72-1.58 (m, 4H), 1.58-0.95 (m, 1H), 0.93-0.86 (m, 1H), 0.84 (d, 3H). |
| 153 | 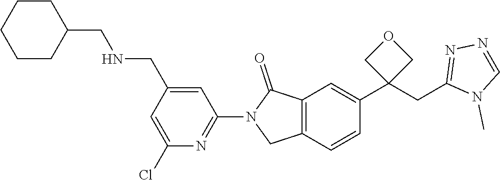 [SMILES: Cn1cnnc1CC1(c2ccc3c(c2)C(=O)N(c2cc(CNCC4CCCCC4)cc(Cl)n2)C3)COC1] Starting materials: Int-1 and Int-178 Reaction conditions: A″ | LCMS: 523.2 [M + 2 + H]⁺. ¹H-NMR (400 MHz, DMSO-d6): δ 8.48 (s, 1H), 8.21 (s, 1H), 7.62 (d, 1H), 7.51 (d, 1H), 7.36 (dd, 1H), 7.26 (s, 1H), 5.03 (s, 2H), 4.98 (d, 2H), 4.91 (d, 2H), 3.77 (s, 2H), 3.57 (s, 2H), 3.03 (s, 3H), 1.82-1.72 (m, 2H), 1.71-1.56 (m, 3H), 1.53-1.34 (m, 1H) 1.20-1.10 (m, 2H), 0.95-0.78 (m, 3H). |
| 154 | 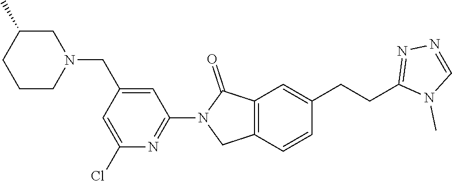 [SMILES: C[C@H]1CCCN(Cc2cc(Cl)nc(N3Cc4ccc(CCc5nncn5C)cc4C3=O)c2)C1] Starting materials: Int-127 and Int-18 Reaction conditions: A″ | LCMS: 465.2 [M + H]⁺. ¹H-NMR (400 MHz, DMSO-d6): δ 8.51 (d, 1H), 8.34 (s, 1H), 7.73 (s, 1H), 7.63 (s, 2H), 7.20 (d, 1H), 5.03 (s, 2H), 3.55 (s, 3H), 3.54 (s, 2H), 3.18-3.12 (m, 2H), 3.08-3.02 (m, 2H), 2.76-2.70 (m, 2H), 2.04-1.89 (m, 1H), 1.61-1.58 (m, 4H), 1.57-1.46 (m, 1H), 0.92-0.85 (m, 1H), 0.84 (d, 3H). |
| 155 | 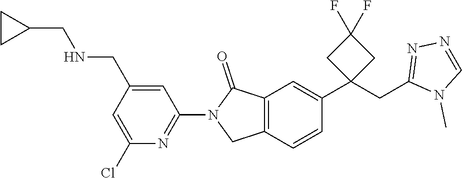 [SMILES: Cn1cnnc1CC1(c2ccc3c(c2)C(=O)N(c2cc(CNCC4CC4)cc(Cl)n2)C3)CC(F)(F)C1] Starting materials: Int-76 and Int-124 Reaction conditions: A″ | LCMS: 513.2 [M + H]⁺. ¹H-NMR (400 MHz, DMSO): δ 8.48 (d, 1H), 8.20 (s, 1H), 7.61 (d, 1H), 7.50 (d, 1H), 7.36 (dd, 1H), 7.27 (d, 1H), 5.03 (s, 2H), 3.81 (s, 2H), 3.35-3.25 (m, 4H), 3.11- 3.00 (m, 2H), 2.80 (s, 3H), 2.38 (d, 2H), 0.93-0.86 (m, 1H), 0.43-0.39 (m, 2H), 0.13-0.10 (m, 2H). |
| 156 | 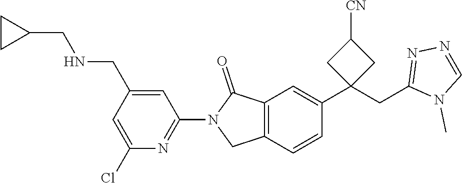 [SMILES: Cn1cnnc1CC1(c2ccc3c(c2)C(=O)N(c2cc(CNCC4CC4)cc(Cl)n2)C3)CC(C#N)C1] Starting materials: Int-23 and Int-76 Reaction conditions: A″ | LCMS: 502.2 [M + H]⁺. ¹H-NMR (400 MHz, DMSO-d₆): δ 8.57 (s, 1H), 8.18 (s, 1H), 8.02-7.98 (m, 1H), 7.68-7.60 (m, 2H), 7.29 (d, 1H), 5.12 (s, 2H), 4.05 (s, 2H), 3.48 (s, 2H), 3.09-3.02 (d, 4H), 2.89 (s, 3H), 2.70 (d, 2H), 2.10-2.02 (m, 1H), 1.68-1.59 (m, 1H), 0.68-0.60 (m, 2H), 0.34-0.28 (m, 2H). |
| 157 | 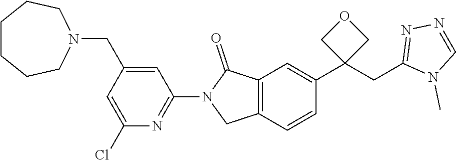 [SMILES: Cn1cnnc1CC1(c2ccc3c(c2)C(=O)N(c2cc(CN4CCCCCC4)cc(Cl)n2)C3)COC1] Starting materials: Int-1 and Int-179 Reaction conditions: A″ | LCMS: 507.23 [M + H]⁺; ¹H-NMR (400 MHz, DMSO-d₆): δ 8.57 (s, 1H), 8.21 (s, 1H), 7.62 (d, 1H), 7.53 (s, 1H), 7.36 (dd, 1H), 7.20 (s, 1H), 5.03 (s, 2H), 4.97 (d, 2H), 4.91 (d, 2H), 3.72 (s, 2H), 3.57 (s, 2H), 3.02 (s, 3H), 2.62- 2.60 (m, 4H), 1.68-1.59 (m, 8H). |
| 158 | 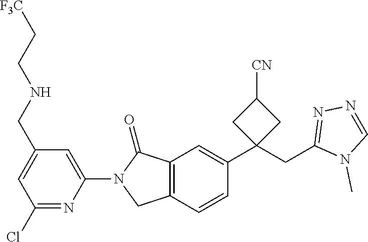 [SMILES: Cn1cnnc1CC1(c2ccc3c(c2)C(=O)N(c2cc(CNCCC(F)(F)F)cc(Cl)n2)C3)CC(C#N)C1] Starting materials: Int-23 and Int-106 Reaction conditions: A″ | LCMS: 544.1 [M + H]⁺; ¹H-NMR (400 MHz, DMSO-d₆): δ 8.47 (s, 1H), 8.19 (s, 1H), 7.62 (d, 1H), 7.51 (d, 1H), 7.40 (dd, 1H), 7.27 (s, 1H), 5.03 (s, 2H), 3.81 (s, 2H), 3.36 (s, 2H), 3.28-3.20 (m, 1H), 2.95-2.88 (m, 4H), 2.88 (s, 3H), 2.79-2.70 (m, 3H), 2.49-2.40 (m, 1H). |
| 159 | 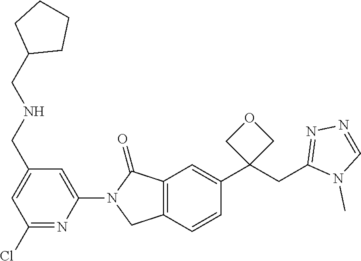 [SMILES: Cn1cnnc1CC1(c2ccc3c(c2)C(=O)N(c2cc(CNCC4CCCC4)cc(Cl)n2)C3)COC1] Starting materials: Int-1 and Int-180 Reaction conditions: A″ | LCMS: 507.2 [M + H]⁺; ¹H-NMR (400 MHz, DMSO-d6): δ 8.49 (s, 1H), 8.21 (s, 1H), 7.62 (d, 1H), 7.51 (d, 1H), 7.36 (dd, 1H), 7.26 (s, 1H), 5.03 (s, 2H), 4.97 (d, 2H), 4.91 (d, 2H), 3.79 (s, 2H), 3.57 (s, 2H), 3.03 (s, 3H), 2.42 (d, 2H), 2.04-1.94 (m, 1H), 1.77-1.66 (m, 2H), 1.60-1.42 (m, 4H), 1.35- 1.12 (m, 2H). |
| 160 | 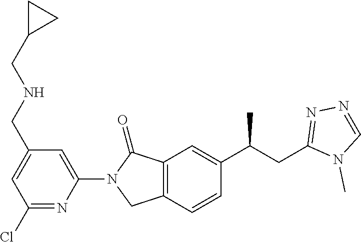 [SMILES: C[C@@H](Cc1nncn1C)c1ccc2c(c1)C(=O)N(c1cc(CNCC3CC3)cc(Cl)n1)C2] Enantiomer 1 Starting materials: Int-2 and Int-76 Reaction conditions: A″ | LCMS: 451.4 [M + H]⁺; ¹HNMR (400 MHz, CD₃OD): δ 8.51 (d, 1H), 8.29 (s, 1H), 7.76 (s, 1H), 7.59-7.58 (m, 2H), 7.24 (s, 1H), 5.07 (s, 2H), 3.89 (s, 2H), 3.52 (s, 3H), 3.50-3.48 (m, 1H), 3.10-3.23 (m, 2H), 2.51 (d, 2H), 1.47 (d, 3H), 1.04-1.00 (m, 1H), 0.57- 0.52 (m, 2H), 0.25-0.17 (m, 2H). |
| 161 | 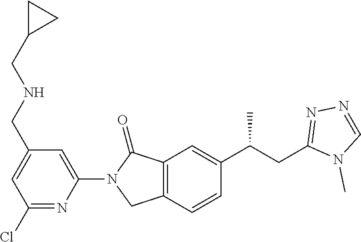 [SMILES: C[C@H](Cc1nncn1C)c1ccc2c(c1)C(=O)N(c1cc(CNCC3CC3)cc(Cl)n1)C2] Enantiomer 2 Starting materials: Int-2 and Int-76 Reaction conditions: A″ | LCMS: 451.4 [M + H]⁺; ¹HNMR (400 MHz, CD₃OD): δ 8.51 (d, 1H), 8.29 (s, 1H), 7.76 (s, 1H), 7.61-7.56 (m, 2H), 7.24 (s, 1H), 5.06 (s, 2H), 3.89 (s, 2H), 3.52 (s, 3H), 3.50-3.46 (m, 1H), 3.22-3.10 (m, 2H), 2.51 (d, 2H), 1.46 (d, 3H), 1.04-1.00 (m, 1H), 0.57- 0.52 (m, 2H), 0.22-0.18 (m, 2H). |
| 162 | 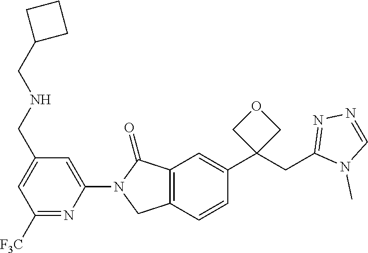 [SMILES: Cn1cnnc1CC1(c2ccc3c(c2)C(=O)N(c2cc(CNCC4CCC4)cc(C(F)(F)F)n2)C3)COC1] Starting materials: Int-1 and Int-181 Reaction conditions: A″ | LCMS: 527.2 [M + H]⁺; ¹H-NMR (400 MHz, DMSO-d6): δ 8.78 (s, 1H), 8.21 (s, 1H), 7.66-7.64 (m, 2H), 7.53 (d, 1H), 7.37 (dd, 1H), 5.07 (s, 2H), 4.98 (d, 2H), 4.91 (d, 2H), 3.86 (s, 2H), 3.58 (s, 2H), 3.03 (s, 3H), 2.49-2.40 (m, 1H), 2.01-1.98 (m, 2H), 1.86-1.75 (m, 2H), 1.72-1.62 (m, 2H). |
| 163 | 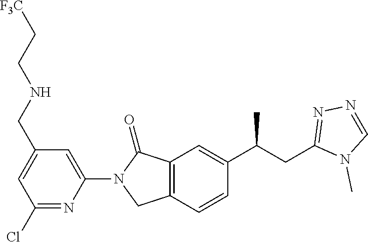 [SMILES: C[C@@H](Cc1nncn1C)c1ccc2c(c1)C(=O)N(c1cc(CNCCC(F)(F)F)cc(Cl)n1)C2] Enantiomer 1 Starting materials: Int-2 and Int-106 Reaction conditions: A″ | LCMS: 495.1 [M + 2 + H]⁺; ¹HNMR (400 MHz, CD₃OD): δ 8.54 (d, 1H), 8.29 (s, 1H), 7.77 (s, 1H), 7.62-7.57 (m, 2H), 7.25 (s, 1H), 5.08 (s, 2H), 3.88 (s, 2H), 3.52 (s, 3H), 3.51-3.46 (m, 1H), 3.22-3.10 (m, 2H), 2.88 (dd, 2H), 2.50-2.40 (m, 2H), 1.47 (d, 3H). |
| 164 | 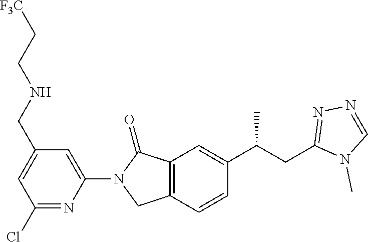 [SMILES: C[C@H](Cc1nncn1C)c1ccc2c(c1)C(=O)N(c1cc(CNCCC(F)(F)F)cc(Cl)n1)C2] Enantiomer 2 Starting materials: Int-2 and Int-106 Reaction conditions: A″ | LCMS: 495.1 [M + 2 + H]⁺; ¹HNMR (400 MHz, CD₃OD): δ 8.53 (d, 1H), 8.29 (s, 1H), 7.77 (s, 1H), 7.62-7.57 (m, 2H), 7.24 (s, 1H), 5.08 (s, 2H), 3.88 (s, 2H), 3.52 (s, 3H), 3.50-3.49 (m, 1H), 3.22-3.10 (m, 2H), 2.88 (dd, 2H), 2.49-2.44 (m, 2H), 1.47 (d, 3H). |
| 165 | 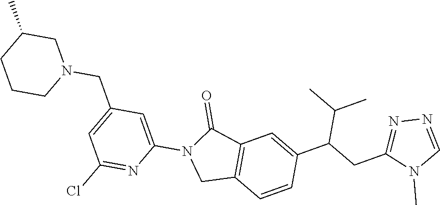 [SMILES: CC(C)C(Cc1nncn1C)c1ccc2c(c1)C(=O)N(c1cc(CN3CCC[C@H](C)C3)cc(Cl)n1)C2] Starting materials: Int-120 and Int-18 Reaction conditions: A″ | LCMS: 507.2 [M + H]⁺; ¹H-NMR (400 MHz, DMSO-d6): δ 8.50 (s, 1H), 8.19 (s, 1H), 7.61 (s,1H), 7.59-7.52 (m, 2H), 7.19 (s, 1H), 5.00 (s, 2H), 3.52 (s, 2H), 3.46 (s, 3H), 3.22-3.12 (m, 3H), 2.78- 2.69 (m, 2H), 2.34-1.92 (m, 2H), 1.70- 1.44 (m, 5H), 1.01 (d, 3H), 0.90-0.85 (m, 1H), 0.84 (d, 3H), 0.75 (d, 3H). |
| 166 | 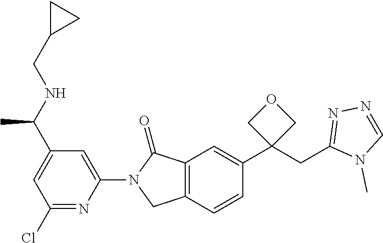 [SMILES: C[C@@H](NCC1CC1)c1cc(Cl)nc(N2Cc3ccc(C4(Cc5nncn5C)COC4)cc3C2=O)c1] Enantiomer 1 Starting materials: Int-1 and Int-103 Reaction conditions: A″ | LCMS: 493.2 [M + H]⁺; ¹HNMR (400 MHz, DMSO-d₆): δ 8.47 (s, 1H), 8.21 (s, 1H), 7.62 (d, 1H), 7.49 (s, 1H), 7.39 - 7.33 (m, 1H), 7.28 (s, 1H), 5.02 (s, 2H), 4.98 (d, 2H), 4.90 (d, 2H), 3.85- 3.79 (m, 1H), 3.57 (s, 2H), 3.00 (s, 3H), 2.30-2.23 (m, 1H), 2.20-2.11 (m, 1H), 1.27 (d, 3H), 0.88-0.84 (m, 1H), 0.41-0.32 (m, 2H), 0.07-0.05 (m, 2H). |
| 167 | 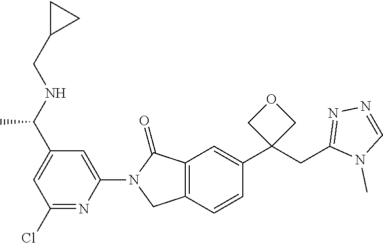 [SMILES: C[C@H](NCC1CC1)c1cc(Cl)nc(N2Cc3ccc(C4(Cc5nncn5C)COC4)cc3C2=O)c1] Enantiomer 2 Starting materials: Int-1 and Int-103 Reaction conditions: A″ | LCMS: 493.3 [M + H]⁺; ¹HNMR (400 MHz, DMSO-d₆): δ 8.47 (s, 1H), 8.21 (s, 1H), 7.62 (d, 1H), 7.49 (d, 1H), 7.35 (dd, 1H), 7.28 (s, 1H), 5.02 (s, 2H), 4.98 (d, 2H), 4.91 (d, 2H), 3.83-3.80 (m, 1H), 3.57 (s, 2H), 3.00 (s, 3H), 2.30-2.21 (m, 2H), 2.19-2.16 (m, 1H), 1.27 (d, 3H), 0.88-0.80 (m, 1H), 0.40-0.30 (m, 2H), 0.08 0.05 (m, 2H). |
| 168 | 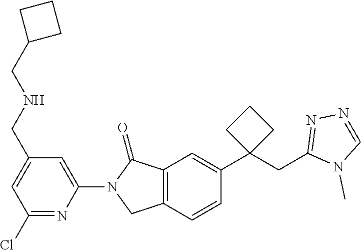 [SMILES: Cn1cnnc1CC1(c2ccc3c(c2)C(=O)N(c2cc(CNCC4CCC4)cc(Cl)n2)C3)CCC1] Starting materials: Int-3 and Int-172 Reaction conditions: A″ | LCMS: 491.15 [M + H]⁺; ¹HNMR (400 MHz, DMSO-d₆): δ 8.48 (s, 1H), 8.15 (s, 1H), 7.57 (d, 1H), 7.33 (s, 1H), 7.25 (s, 1H), 7.22 (dd, 1H), 5.01 (s, 2H), 3.76 (s, 2H), 3.25 (s, 2H), 2.76 (s, 3H), 2.60-2.54 (m, 3H), 2.45-2.34 (m, 2H), 2.24-2.15 (m, 1H), 2.05-1.93 (m, 3H), 1.89-1.77 (m, 3H), 1.71- 1.61 (m, 2H). |
| 169 | 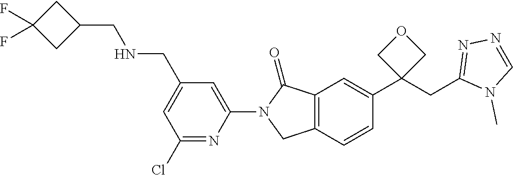 [SMILES: Cn1cnnc1CC1(c2ccc3c(c2)C(=O)N(c2cc(CNCC4CC(F)(F)C4)cc(Cl)n2)C3)COC1] Starting materials: Int-1 and Int-182 Reaction conditions: A″ | LCMS: 529.3 [M + H]⁺; ¹HNMR (400 MHz, DMSO-d₆): δ 8.49 (s, 1H), 8.21 (s, 1H), 7.62 (d, 1H), 7.52 (s, 1H), 7.36 (dd, 1H), 7.26 (s, 1H), 5.03 (s, 2H), 4.97 (d, 2H), 4.91 (d, 2H), 3.79 (s, 2H), 3.57 (s, 2H), 3.03 (s, 3H), 2.70-2.58 (m, 4H), 2.25-2.35 (m, 3H). |
| 170 | 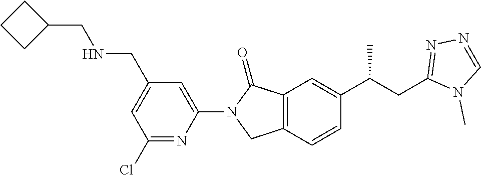 [SMILES: C[C@H](Cc1nncn1C)c1ccc2c(c1)C(=O)N(c1cc(CNCC3CCC3)cc(Cl)n1)C2] Enantiomer 1 Starting materials: Int-2 and Int-172 Reaction conditions: A″ | LCMS: 465.3 [M + H]⁺; ¹HNMR (400 MHz, CD₃OD): δ 8.51 (s, 1H), 8.29 (s, 1H), 7.77 (s, 1H), 7.61-7.58 (m, 2H), 7.23 (s, 1H), 5.08 (s, 2H), 3.84 (s, 2H), 3.52 (s, 3H), 3.50-3.47 (m, 1H), 3.21 - 3.10 (m, 2H), 2.66 (d, 2H), 2.58- 2.54 (m, 1H), 2.15-2.10 (m, 2H), 1.98 - 1.89 (m, 2H), 1.77-1.72 (m, 2H), 1.47 (d, 3H). |
| 171 | 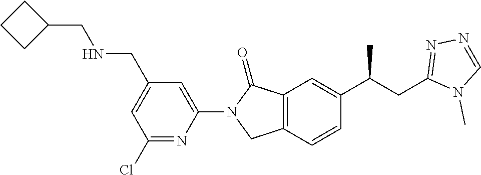 [SMILES: C[C@@H](Cc1nncn1C)c1ccc2c(c1)C(=O)N(c1cc(CNCC3CCC3)cc(Cl)n1)C2] Enantiomer 2 Starting materials: Int-2 and Int-172 Reaction conditions: A″ | LCMS: 465.3 [M + H]⁺; ¹HNMR (400 MHz, CD₃OD): δ 8.51 (s, 1H), 8.29 (s, 1H), 7.77 (s, 1H), 7.52-7.58 (m, 2H), 7.23 (s, 1H), 5.08 (s, 2H), 3.84 (s, 2H), 3.52 (s, 3H), 3.51-3.45 (m, 1H), 3.22 - 3.10 (m, 2H), 2.65 (d, 2H), 2.63- 2.50 (m, 1H), 2.14-2.12 (m, 2H), 2.01 - 1.83 (m, 2H), 1.80-1.70 (m, 2H), 1.47 (d, 3H). |
| 172 | 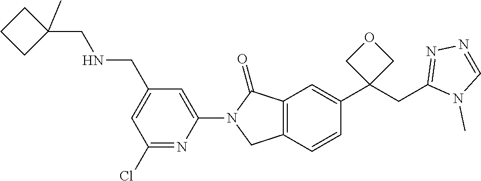 [SMILES: Cn1cnnc1CC1(c2ccc3c(c2)C(=O)N(c2cc(CNCC4(C)CCC4)cc(Cl)n2)C3)COC1] Starting materials: Int-1 and Int-155 Reaction conditions: A″ | LCMS: 507.3 [M + H]⁺; ¹HNMR (400 MHz, DMSO-d6): δ 8.52 (s, 1H), 8.21 (s, 1H), 7.62 (d, 1H), 7.52 (s, 1H), 7.35 (dd, 1H), 7.27 (s, 1H), 5.03 (s, 2H), 4.97 (d, 2H), 4.91 (d, 2H), 3.82 (s, 2H), 3.57 (s, 2H), 3.03 (s, 3H), 2.43 (s, 2H), 1.87-1.86 (m, 3H), 1.80-1.72 (m, 1H), 1.65-1.57 (m, 2H), 1.14 (s, 3H). |
| 173 | 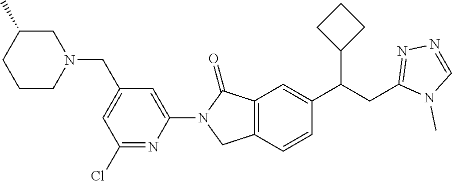 [SMILES: C[C@H]1CCCN(Cc2cc(Cl)nc(N3Cc4ccc(C(Cc5nncn5C)C5CCC5)cc4C3=O)c2)C1] Starting materials: Int-122 and Int-18 Reaction conditions: A″ | LCMS: 521.2 [M + H]⁺; +HNMR (400 MHz, DMSO-d6): δ 8.50 (s, 1H), 8.21 (s, 1H), 7.62 (s, 1H), 7.58-7.54 (m, 2H), 7.18 (s, 1H), 5.00 (s, 2H), 3.53 (s, 2H), 3.42 (s, 3H), 3.28-3.20 (m, 1H), 3.10-2.90 (m, 2H), 2.75-2.70 (m, 2H), 2.10-1.90 (m, 2H), 1.8-1.45 (m, 11H), 0.90-0.85 (m, 1H), 0.84 (d, 3H). |
| 174 | 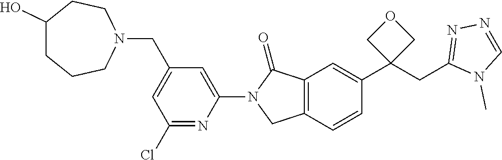 [SMILES: Cn1cnnc1CC1(c2ccc3c(c2)C(=O)N(c2cc(CN4CCCC(O)CC4)cc(Cl)n2)C3)COC1] Starting materials: Int-1 and Int-183 Reaction conditions: A″ | LCMS: 523.2 [M + H]⁺; ¹HNMR (400 MHz, DMSO-d₆): δ 8.54 (s, 1H), 8.21 (s, 1H), 7.62 (d, 1H), 7.52 (s, 1H), 7.36 (dd, 1H), 7.20 (s, 1H), 5.02 (s, 2H), 4.97 (d, 2H), 4.92 (d, 2H), 4.41 (d, 1H), 3.78-3.75 (m, 1H), 3.68 (s, 2H), 3.57 (s, 2H), 3.03 (s, 3H), 2.70-2.59 (m, 3H), 2.49-2.47 (m, 1H), 1.80- 1.50 (m, 2H), 1.75-1.68 (m, 1H), 1.65 - 1.56 (m, 2H), 1.53-1.43 (m, 1H). |
| 175 | 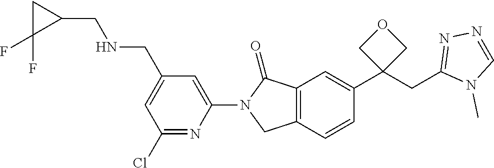 [SMILES: Cn1cnnc1CC1(c2ccc3c(c2)C(=O)N(c2cc(CNCC4CC4(F)F)cc(Cl)n2)C3)COC1] Starting materials: Int-1 and Int-184 Reaction conditions: A″ | LCMS: 515.2 [M + H]⁺; ¹HNMR (400 MHz, DMSO-d₆): δ 8.48 (s, 1H), 8.21 (s, 1H), 7.62 (d, 1H), 7.50 (d, 1H), 7.36 (dd, 1H), 7.27 (s, 1H), 5.03 (s, 2H), 4.98 (d, 2H), 4.91 (d, 2H), 3.88-3.78 (m, 2H), 3.57 (s, 2H), 3.03 (s, 3H), 2.68-2.56 (m, 2H), 1.90-1.80 (m, 1H), 1.605-1.50 (m, 1H), 1.24-1.15 (m, 1H). |
| 176 | 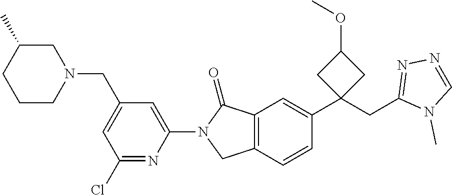 [SMILES: COC1CC(Cc2nncn2C)(c2ccc3c(c2)C(=O)N(c2cc(CN4CCC[C@H](C)C4)cc(Cl)n2)C3)C1] Starting materials: Int-23 and Int-18 Reaction conditions: D″ | LCMS: 535.2 [M + H]⁺; ¹H-NMR (400 MHz, CD3OD): δ 8.18 (s, 1H), 7.47 (d, 1H), 7.40 (d, 1H), 7.13 (dd, 1H), 7.02 (s, 1H), 7.69 (s, 1H), 5.52-5.47 (m, 1H), 4.49 (s, 2H), 3.36 (s, 2H), 3.34 (s, 2H), 3.19 (s, 3H), 2.84-2.77 (m, 2H), 2.76 (s, 3H), 2.52-2.47 (m, 2H), 2.01 - 1.92 (m, 1H), 1.80-1.60 (m, 5H), 0.95-0.87 (m, 1H), 0.88 (d, 3H). |
| 177 | 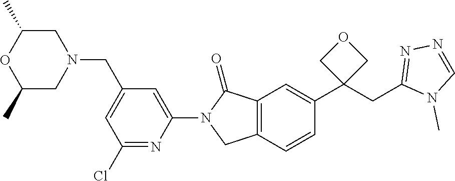 [SMILES: C[C@@H]1CN(Cc2cc(Cl)nc(N3Cc4ccc(C5(Cc6nncn6C)COC5)cc4C3=O)c2)C[C@@H](C)O1] Starting materials: Int-1 and Int-185 Reaction conditions: A″ | LCMS: 523.2 [M + H]⁺; ¹H-NMR (400 MHz, DMSO-d6): δ 8.57 (s, 1H), 8.21 (s, 1H), 7.61 (d, 1H) , 7.55 (d, 1H), 7.36 (dd, 1H), 7.21 (s, 1H), 5.03 (s, 2H), 5.00 (d, 2H), 4.91 (d, 2H), 3.90- 4.00 (m, 2H), 3.58 (d, 1H), 3.57 (s,1H), 3.48 (d, 1H), 3.03 (s, 3H), 2.48-2.41 (m, 2H), 2.1-2.2 (m, 2H), 1.18 (d, 6H). |
| 178 | 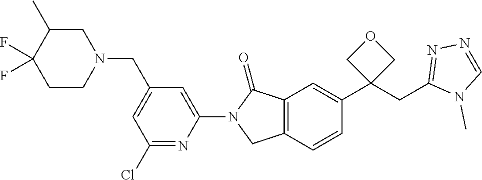 [SMILES: CC1CN(Cc2cc(Cl)nc(N3Cc4ccc(C5(Cc6nncn6C)COC5)cc4C3=O)c2)CCC1(F)F] Starting materials: Int-1 and Int-157 Reaction conditions: A″ | LCMS: 543.2 [M + H]⁺; ¹H-NMR (400 MHz, DMSO-d6): δ 8.51 (s, 1H), 8.22 (s, 1H), 7.62 (d, 1H), 7.52 (d, 1H), 7.37 (dd, 1H), 7.23 (s, 1H), 5.03 (s, 2H), 4.97 (d, 2H), 4.91 (d, 2H), 3.65 (s, 2H), 3.57 (s, 2H), 3.04 (s, 3H), 2.80- 2.65 (m, 2H), 2.40-2.30 (m, 1H), 2.20 - 1.86 (m, 4H), 0.97 (d, 3H). |
| 179 | 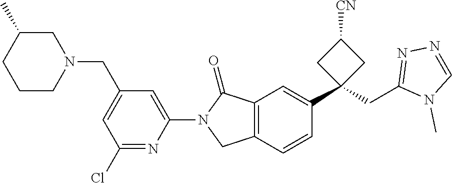 [SMILES: C[C@H]1CCCN(Cc2cc(Cl)nc(N3Cc4ccc([C@]5(Cc6nncn6C)C[C@@H](C#N)C5)cc4C3=O)c2)C1] Enantiomer 1 Starting materials: Int-18 and Int-23 Reaction conditions: A″ | LCMS: 530.2 [M + H]⁺; ¹H-NMR (400 MHz, DMSO-d6): δ 8.49 (s, 1H), 8.17 (s, 1H), 7.59 (d, 1H), 7.35 (d, 1H), 7.25-7.16 (m, 2H), 5.02 (s, 2H), 3.82 - 3.72 (m, 1H), 3.53 (s, 2H), 3.29 (s, 2H), 3.12-3.04 (m, 2H), 2.76 (s, 3H), 2.76-2.60 (m, 4H), 2.00-1.90 (m, 1H), 1.72-1.45 (m, 5H), 0.90-0.84 (m, 1H), 0.84 (d, 3H). |
| 180 | 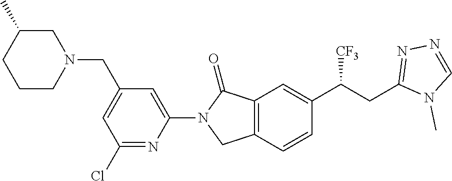 [SMILES: C[C@H]1CCCN(Cc2cc(Cl)nc(N3Cc4ccc([C@@H](Cc5nncn5C)C(F)(F)F)cc4C3=O)c2)C1] Enantiomer 1 Starting materials: Int-119 and Int-18 Reaction conditions: A″ | LCMS: 533.2 [M + H]⁺; ¹H-NMR (400 MHz, DMSO): δ 8.18 (s, 1H), 7.47 (d, 1H), 7.40 (d, 1H), 7.13 (dd, 1H), 7.02 (s, 1H), 7.69 (s, 1H), 5.05 (s, 2H), 4.60 - 4.49 (m, 1H), 3.65-3.60 (m, 1H), 3.60 (s, 3H), 3.54 (s, 2H), 3.50-3.42 (m, 1H), 2.74-2.68 (m, 2H), 2.01- 1.92 (m, 1H), 1.70-1.40 (m, 5H), 0.92-0.87 (m, 4H). |
| 181 | 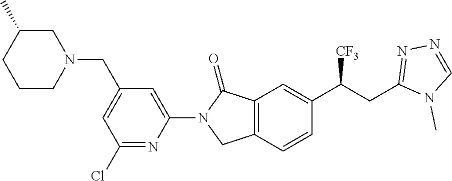 [SMILES: C[C@H]1CCCN(Cc2cc(Cl)nc(N3Cc4ccc([C@H](Cc5nncn5C)C(F)(F)F)cc4C3=O)c2)C1] Enantiomer 2 Starting materials: Int-119 and Int-18 Reaction conditions: A″ | LCMS: 533.2 [M + H]⁺; ¹H-NMR (400 MHz, DMSO): δ 8.50 (s, 1H), 8.28 (s, 1H), 7.95 (s, 1H), 7.84 (d, 1H), 7.70 (d, 1H), 7.20 (s, 1H), 5.05 (s, 2H), 4.57-4.45 (m, 1H), 3.65-3.60 (m, 1 H), 3.60 (s, 3H), 3.54 (s, 2H), 3.53- 3.45 (m, 1H), 2.75-2.70 (m, 2H), 2.01 - 1.90 (m, 1H), 1.70-1.60 (m, 4H), 1.60-1.40 (m, 1H), 0.86-0.83 (m, 4H). |
| 182 | 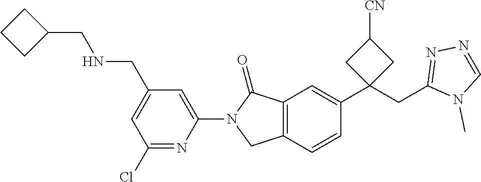 [SMILES: Cn1cnnc1CC1(c2ccc3c(c2)C(=O)N(c2cc(CNCC4CCC4)cc(Cl)n2)C3)CC(C#N)C1] Starting materials: Int-23 and Int-172 Reaction conditions: A″ | LCMS: 518.2 [M + 2 + H]⁺; ¹H-NMR (400 MHz, DMSO-d6): δ 8.48 (s, 1H), 8.19 (s, 1H), 7.61 (d, 1H), 7.51 (d, 1H), 7.39 (dd, 1H), 7.25 (s, 1H), 5.02 (s, 2H), 3.76 (s, 2H), 3.36 (s, 2H), 3.30- 3.20 (m, 1H), 2.92 (s, 2H), 2.90 (s, 2H), 2.87 (s, 3H), 2.05-1.95 (m, 2H), 1.90-1.74 (m, 2H), 1.72-1.60 (m, 2H). |
| 183 | 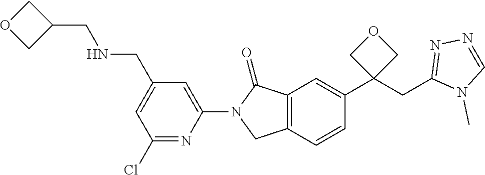 [SMILES: Cn1cnnc1CC1(c2ccc3c(c2)C(=O)N(c2cc(CNCC4COC4)cc(Cl)n2)C3)COC1] Starting materials: Int-1 and Int-186 Reaction conditions: A″ | LCMS: 495.2 [M + H]⁺; ¹H-NMR (400 MHz, DMSO-d6): δ 8.50 (d, 1H), 8.21 (s, 1H), 7.62 (d, 1H), 7.52 (d, 1H), 7.36 (dd, 1H), 7.25 (s, 1H), 5.03 (s, 2H), 4.97 (d, 2H), 4.91 (d, 2H), 4.63 (dd, 2H), 4.29 (dd, 2H), 3.79 (s, 2H), 3.57 (s, 2H), 3.10-3.0 (m, 1H), 3.03 (s, 3H), 2.78 (d, 2H). |
| 184 | 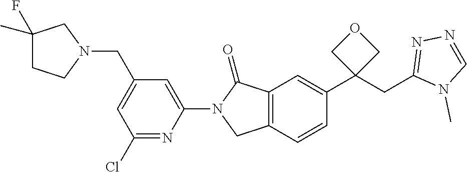 [SMILES: Cn1cnnc1CC1(c2ccc3c(c2)C(=O)N(c2cc(CN4CCC(C)(F)C4)cc(Cl)n2)C3)COC1] Starting materials: Int-1 and Int-158 Reaction conditions: A″ | LCMS: 511.2 [M + H]⁺; ¹H-NMR (400 MHz, DMSO-d₆): δ 8.49 (s, 1H), 8.21 (s, 1H), 7.62 (d, 1H), 7.50 (d, 1H), 7.37 (dd, 1H), 7.22 (s, 1H), 5.03 (s, 2H), 4.98 (d, 2H), 4.91 (d, 2H), 3.72 (s, 2H), 3.57 (s, 2H), 3.02 (s, 3H), 2.89-2.80 (m, 2H), 2.67-2.56 (m, 2H), 2.15- 1.87 (m, 2H), 1.34 (d, 3H). |
| 185 | 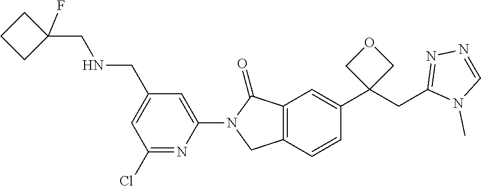 [SMILES: Cn1cnnc1CC1(c2ccc3c(c2)C(=O)N(c2cc(CNCC4(F)CCC4)cc(Cl)n2)C3)COC1] Starting materials: Int-1 and Int-187 Reaction conditions: A″ | LCMS: 511.2 [M + H]⁺; ¹H-NMR (400 MHz, DMSO-d6): δ 8.52 (d, 1H), 8.21 (s, 1H), 7.62 (d, 1H) , 7.52 (d, 1H), 7.36 (dd, 1H), 7.27 (d, 1H), 5.03 (s, 2H), 4.97 (d, 2H), 4.91 (d, 2H), 3.86 (s, 2H), 3.57 (s, 2H), 3.03 (s, 3H), 2.76 (d, 2H), 2.19-2.23 (m, 4H), 1.66-1.73 (m, 1H), 1.35-1.51 (m, 1H). |
| 186 | 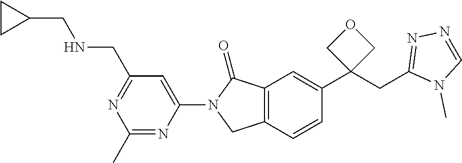 [SMILES: Cc1nc(CNCC2CC2)cc(N2Cc3ccc(C4(Cc5nncn5C)COC4)cc3C2=O)n1] Starting materials: Int-1 and Int-165 Reaction conditions: A″ | LCMS: 460.2 [M + H]⁺; ¹H-NMR (400 MHz, CD3OD): δ 8.45 (s, 1H), 8.20 (s, 1H), 7.64 (d, 1H), 7.52 (s, 1H), 7.31 (dd, 1H), 5.14 (s, 2H), 5.12 (d, 2H), 5.08 (d, 2H), 4.02 (s, 2H), 3.70 (s, 2H), 2.99 (s, 3H), 2.67-2.65 (m, 5H), 1.10 - 1.05 (m, 1H), 0.63-0.55 (m, 2H), 0.30-0.28 (m, 2H). |
| 187 | 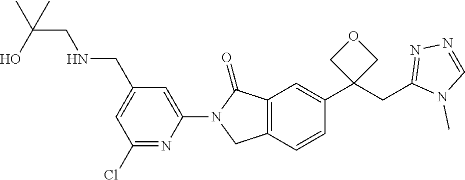 [SMILES: Cn1cnnc1CC1(c2ccc3c(c2)C(=O)N(c2cc(CNCC(C)(C)O)cc(Cl)n2)C3)COC1] Starting materials: Int-1 and Int-188 Reaction conditions: A″ | LCMS: 497.2 [M + H]⁺; ¹H-NMR (400 MHz, DMSO-d₆): δ 8.50 (s, 1H), 8.21 (s, 1H), 7.62 (d, 1H), 7.52 (s, 1H), 7.36 (dd, 1H), 7.27 (s, 1H), 5.02 (s, 1H), 4.97 (d, 2H), 4.91 (d, 2H), 3.84 (s, 2H), 3.57 (s, 2H), 3.02 (s, 3H), 2.40 (s, 2H), 1.13 (s, 6H). |
| 188 | 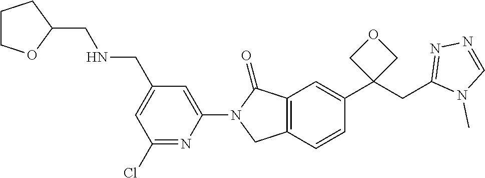 [SMILES: Cn1cnnc1CC1(c2ccc3c(c2)C(=O)N(c2cc(CNCC4CCCO4)cc(Cl)n2)C3)COC1] Starting materials: Int-1 and Int-189 Reaction conditions: A″ | LCMS: 509.2 [M + H]⁺; ¹H-NMR (400 MHz, DMSO-d6): δ 8.48 (s, 1H), 8.21 (s, 1H), 7.62 (d, 1H), 7.51 (d, 1H), 7.36 (dd, 1H), 7.26 (s, 1H), 5.03 (s, 2H), 4.98 (d, 2H), 4.91 (d, 2H), 3.95- 3.84 (m, 1H), 3.77 (s, 2H), 3.80-3.70 (m, 1H), 3.65-3.58 (m, 1H), 3.57 (s, 2H), 3.02 (s, 3H), 2.54 (d, 2H), 1.98- 1.87 (m, 1H), 1.86-1.74 (m, 2H), 1.62- 1.50 (m, 1H). |
| 189 | 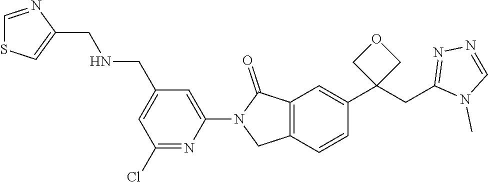 [SMILES: Cn1cnnc1CC1(c2ccc3c(c2)C(=O)N(c2cc(CNCc4cscn4)cc(Cl)n2)C3)COC1] Starting materials: Int-1 and Int-190 Reaction conditions: A″ | LCMS: 522.1 [M + H]⁺; ¹H-NMR (400 MHz, DMSO-d6): δ 9.06 (d, 1H), 8.50 (d, 1H), 8.21 (s, 1H), 7.62 (d, 1H), 7.52 (d, 1H), 7.50 (d, 1H), 7.37 (dd, 1H), 7.29 (s, 1H), 5.03 (s, 2H), 4.98 (d, 2H), 4.91 (d, 2H), 3.86 (s, 2H), 3.84 (s, 2H), 3.57 (s, 2H), 3.02 (s, 3H). |
| 190 | 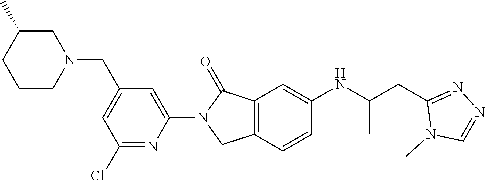 [SMILES: CC(Cc1nncn1C)Nc1ccc2c(c1)C(=O)N(c1cc(CN3CCC[C@H](C)C3)cc(Cl)n1)C2] Starting materials: Int-18 and Int-128 Reaction conditions: A″ | LCMS: 496.2 [M + 2 + H]⁺; ¹H-NMR (400 MHz, DMSO-d₆): δ 8.49 (s, 1H), 8.34 (s, 1H), 7.39 (d, 1H), 7.17 (s, 1H), 6.96 (dd, 1H), 6.90 (d, 1H), 5.98 (d, 1H), 4.88 (s, 2H), 4.13-3.93 (m, 1H), 3.58 (s, 3H), 3.52 (s, 2H), 2.96 (dd, 1H), 2.82 (dd, 1H), 2.77-2.65 (m, 2H), 2.02-1.89 (m, 1H), 1.72-1.56 (m, 4H), 1.55-1.44 (m, 1H), 1.23 (d, 3H), 0.92-0.84 (m, 1H), 0.84 (d, 3H). |
| 191 | 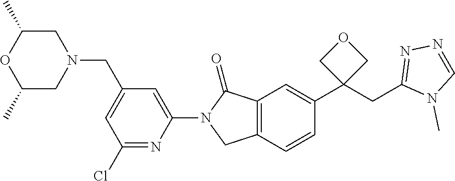 [SMILES: C[C@@H]1CN(Cc2cc(Cl)nc(N3Cc4ccc(C5(Cc6nncn6C)COC5)cc4C3=O)c2)C[C@H](C)O1] Starting materials: Int-1 and Int-193 Reaction conditions: A″ | LCMS: 525.2 [M + 2 + H]⁺; ¹H-NMR (400 MHz, DMSO-d6): δ 8.49 (s, 1H), 8.21 (s, 1H), 7.62 (d, 1H) , 7.51 (s, 1H), 7.36 (dd, 1H), 7.22 (s, 1H), 5.03 (s, 2H), 4.97 (d, 2H), 4.90 (d, 2H), 3.61 - 3.67 (m, 2H), 3.57 (s, 2H), 3.55 (s, 2H), 3.03 (s, 3H), 2.65-2.75 (m, 2H), 1.73 (t, 2H), 1.04 (d, 6H). |
| 192 | 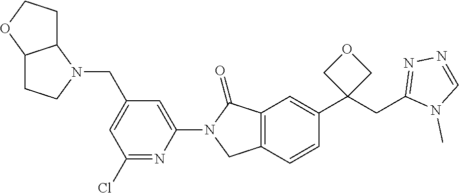 [SMILES: Cn1cnnc1CC1(c2ccc3c(c2)C(=O)N(c2cc(CN4CCC5OCCC54)cc(Cl)n2)C3)COC1] Starting materials: Int-1 and Int-166 Reaction conditions: A″ | LCMS: 521.2 [M + H]⁺; ¹H-NMR (400 MHz, DMSO-d₆): δ 8.52 (s, 1H), 8.21 (s, 1H), 7.62 (d, 1H), 7.51 (d, 1H), 7.37 (dd, 1H), 7.22 (s, 1H), 5.03 (s, 2H), 4.98 (d, 2H), 4.91 (d, 2H), 4.53-4.45 (m, 1H), 3.96 (d, 1H), 3.81-3.75 (m, 1H), 3.75-3.78 (m, 1H), 3.57 (s, 2H), 3.52 (d, 1H), 3.28-3.21 (m, 1H), 2.85 - 2.79 (m, 1H), 2.29-2.20 (m, 1H), 2.08-1.98 (m, 1H), 1.85-1.78 (m, 1H), 1.70-1.55 (m, 2H). |
| 193 | 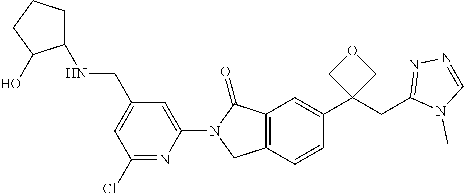 [SMILES: Cn1cnnc1CC1(c2ccc3c(c2)C(=O)N(c2cc(CNC4CCCC4O)cc(Cl)n2)C3)COC1] Starting materials: Int-1 and Int-191 Reaction conditions: A″ | LCMS: 509.1[M + H]⁺; ¹H-NMR (400 MHz, DMSO-d₆): δ 8.48 (s, 1H), 8.21 (s, 1H), 7.62 (d, 1H), 7.50 (s, 1H), 7.37 (dd, 1H), 7.28 (s, 1H), 5.03 (s, 2H), 4.96 (d, 2H), 4.89 (d, 2H), 4.54 (bs, 1H), 3.82-3.75 (m, 3H), 3.57 (s, 2H), 3.02 (s, 3H), 2.80-2.75 (m, 1H), 1.88- 1.79 (m, 2H), 1.62-1.55 (m, 2H), 1.44- 1.38 (m, 1H), 1.37-1.27 (m, 1H). |
| 194 | 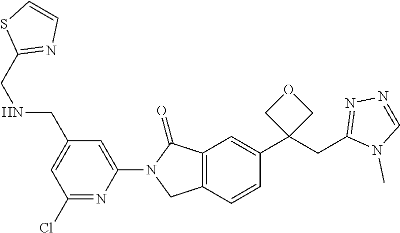 [SMILES: Cn1cnnc1CC1(c2ccc3c(c2)C(=O)N(c2cc(CNCc4nccs4)cc(Cl)n2)C3)COC1] Starting materials: Int-1 and Int-192 Reaction conditions: A″ | LCMS: 522.1 [M + H]⁺; ¹H-NMR (400 MHz, DMSO-d6): δ 8.54 (s, 1H), 8.21 (s, 1H), 7.73 (d, 1H) , 7.66-7.60 (m, 2H), 7.51 (d, 1H), 7.37 (dd, 1H), 7.31 (d, 1H), 5.03 (s, 2H), 5.00 (d, 2H), 4.91 (d, 2H), 4.01 (d, 2H), 3.90 (d, 2H), 3.57 (s, 2H), 3.02 (s, 3H). |
| 195 | 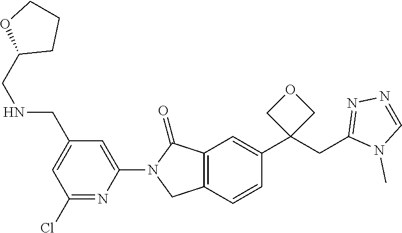 [SMILES: Cn1cnnc1CC1(c2ccc3c(c2)C(=O)N(c2cc(CNC[C@H]4CCCO4)cc(Cl)n2)C3)COC1] Enantiomer 1 Starting materials: Int-1 and Int-189 Reaction conditions: A″ | LCMS: 509.2 [M + H]⁺; ¹H-NMR (400 MHz, CD3OD): δ 8.51 (s, 1H), 8.20 (s, 1H), 7.62 (d, 1H), 7.48 (s, 1H), 7.26- 7.24 (m, 1H), 5.13 (d, 2H), 5.10 (s, 1H), 5.08 (d, 2H), 4.10-4.02 (m, 1H), 3.90 (s, 2H), 3.90-3.85 (m, 1H), 3.80 - 3.76 (m, 1H), 3.70 (s, 2H), 2.96 (s, 3H), 2.72-2.61 (m, 2H), 2.10-2.00 (m, 1H), 1.96-1.88 (m, 2H), 1.67- 1.57 (m, 1H). |
| 196 | 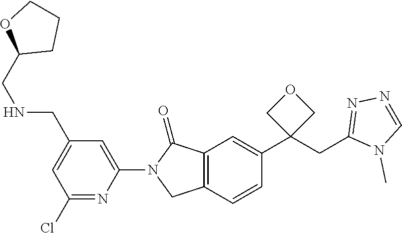 [SMILES: Cn1cnnc1CC1(c2ccc3c(c2)C(=O)N(c2cc(CNC[C@@H]4CCCO4)cc(Cl)n2)C3)COC1] Enantiomer 2 Starting materials: Int-1 and Int-189 Reaction conditions: A″ | LCMS: 509.2 [M + H]⁺; ¹H-NMR (400 MHz, CD3OD): δ 8.51 (s, 1H), 8.20 (s, 1H), 7.62 (d, 1H), 7.48 (s, 1H), 7.26- 7.24 (m, 1H), 5.13 (d, 2H), 5.10 (s, 1H), 5.08 (d, 2H), 4.10-4.02 (m, 1H), 3.90 (s, 2H), 3.90-3.85 (m, 1H), 3.80 - 3.76 (m, 1H), 3.70 (s, 2H), 2.96 (s, 3H), 2.72-2.61 (m, 2H), 2.10-2.00 (m, 1H), 1.96-1.88 (m, 2H), 1.67- 1.57 (m, 1H). |
| 197 | 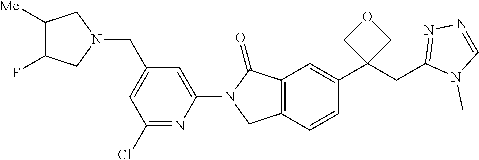 [SMILES: CC1CN(Cc2cc(Cl)nc(N3Cc4ccc(C5(Cc6nncn6C)COC5)cc4C3=O)c2)CC1F] Starting materials: Int-1 and Int-159 Reaction conditions: A″ | LCMS: 511.20 [M + H]⁺; ¹HNMR (400 MHz, DMSO-d₆): δ 8.48 (s, 1H), 8.21 (s, 1H), 7.62 (d, 1H), 7.50 (s, 1H), 7.36 (dd, 1H), 7.21 (s, 1H), 5.11- 5.09 (m, 0.5H), 5.02 (s, 2H), 4.98- 4.94 (m, 2.5H), 4.90 (d, 2H), 3.82- 3.70 (m, 2H), 3.57 (s, 2H), 3.25-3.10 (m, 1H), 3.02 (s, 3H), 2.87-2.80 (m, 1H), 2.78-2.63 (m, 1H), 2.45-2.22 (m, 2H), 1.04-1.00 (m, 3H). |
| 198 | 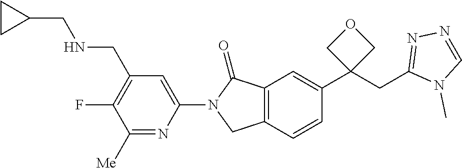 [SMILES: Cc1nc(N2Cc3ccc(C4(Cc5nncn5C)COC4)cc3C2=O)cc(CNCC2CC2)c1F] Starting materials: Int-1 and Int-160 Reaction conditions: A″ | LCMS: 477.2 [M + H]⁺; ¹HNMR (400 MHz, DMSO-d₆): δ 8.50 (d, 1H), 8.21 (s, 1H), 7.61 (d, 1H), 7.48 (s, 1H), 7.34 (dd, 1H), 5.03 (s, 2H), 4.98 (d, 2H), 4.91 (d, 2H), 3.83 (s, 2H), 3.57 (s, 2H), 3.01 (s, 3H), 2.44-2.42 (m, 5H), 0.95-0.92 (m, 1H), 0.43-0.41 (m, 2H), 0.15-0.14 (m, 2H). |
| 199 | 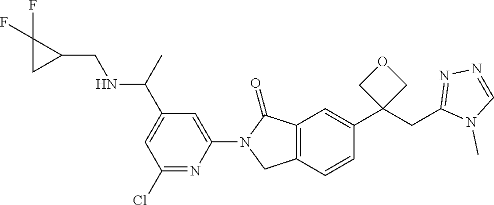 [SMILES: CC(NCC1CC1(F)F)c1cc(Cl)nc(N2Cc3ccc(C4(Cc5nncn5C)COC4)cc3C2=O)c1] Starting materials: Int-1 and Int-194 Reaction conditions: A″ | LCMS: 529.1 [M+ H]+; ¹HNMR (400 MHz, DMSO-d6): δ 8.48 (d, 1H), 8.21 (s, 1H), 7.62 (d, 1H), 7.49 (s, 1H), 7.36 (dd, 1H), 7.30 (d, 1H), 5.02 (s, 2H), 4.98 (d, 2H), 4.91 (d, 2H), 3.84- 3.81 (m, 1H), 3.57 (s, 2H), 3.01 (s, 3H), 2.45-2.30 (m, 1H), 1.88-1.72 (m, 1H), 1.56-1.44 (m, 1H), 1.33- 1.22 (m, 4H), 1.21-1.10 (m, 1H), 0.88-0.86 (m, 1H). |
| 200 | 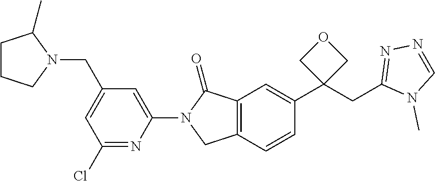 [SMILES: CC1CCCN1Cc1cc(Cl)nc(N2Cc3ccc(C4(Cc5nncn5C)COC4)cc3C2=O)c1] Starting materials: Int-1 and Int-195 Reaction conditions: A″ | LCMS: 493.20 [M + H]⁺; ¹H-NMR (400 MHz, DMSO-d6): δ 8.48 (s, 1H), 8.21 (s, 1H), 7.62 (d, 1H) , 7.49 ( s, 1H), 7.37 (dd, 1H), 7.21 (s, 1H), 5.03 (s, 2H), 4.98 (d, 2H), 4.90 (d, 2H), 4.02 (d, 1H), 3.57 (s, 2H), 3.38 (d, 1H), 3.02 (s, 3H), 2.92-2.83 (m, 1H), 2.48 - 2.42 (m, 1H), 2.13 (q, 1H), 2.01- 1.90 (m, 1H), 1.71-1.60 (m, 2H), 1.43-1.31 (m, 1H), 1.10 (d, 3H). |
| 201 | 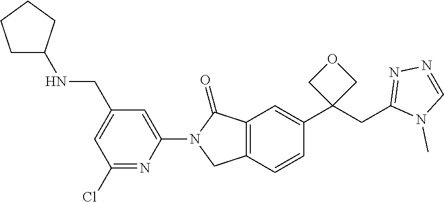 [SMILES: Cn1cnnc1CC1(c2ccc3c(c2)C(=O)N(c2cc(CNC4CCCC4)cc(Cl)n2)C3)COC1] Starting materials: Int-1 and Int-209 Reaction conditions: A″ | LCMS: 493.3 [M + H]⁺; ¹H-NMR (400 MHz, DMSO-d6): δ 8.57 (s, 1H), 8.20 (s, 1H), 7.63 (d, 1H), 7.51 (s, 1H), 7. 42-7.38 (m, 2H), 5.03 (s, 2H), 4.97 (d, 2H), 4.90 (d, 2H), 4.15-4.09 (m, 2H), 3.57 (s, 2H), 3.40-3.35 (m, 1H), 3.05 (s, 3H), 1.95-1.85 (m, 2H), 1.72- 1.65 (m, 2H), 1.60-1.49 (m, 4H). |
| 202 | 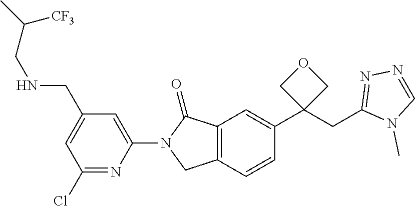 [SMILES: CC(CNCc1cc(Cl)nc(N2Cc3ccc(C4(Cc5nncn5C)COC4)cc3C2=O)c1)C(F)(F)F] Starting materials: Int-1 and Int-196 Reaction conditions: A″ | LCMS: 535.3 [M + 1H]⁺; ¹HNMR (400 MHz, DMSO-d₆): δ 8.50 (s, 1H), 8.21 (s, 1H), 7.62 (d, 1H), 7.52 (s, 1H), 7.37 (dd, 1H), 7.26 (s, 1H), 5.03 (s, 2H), 4.97 (d, 2H), 4.91 (d, 2H), 3.86- 3.78 ( m, 2H), 3.57 (s, 2H), 3.03 (s, 3H), 2.80-2.75 (m, 1H), 2.62-2.55 (m, 2H), , 1.16 (d, 3H). |
| 203 | 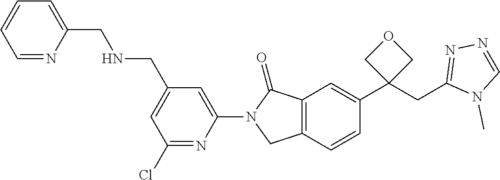 [SMILES: Cn1cnnc1CC1(c2ccc3c(c2)C(=O)N(c2cc(CNCc4ccccn4)cc(Cl)n2)C3)COC1] Starting materials: Int-1 and Int-197 Reaction conditions: A″ | LCMS: 516.2 [M + H]⁺; ¹HNMR (400 MHz, CD3OD): δ 8.54-8.51 (m, 2H), 8.20 (s, 1H), 7.87-7.83 (m, 1H), 7.63 (d, 1H), 7.56 (d, 1H), 7.48 (s, 1H), 7.36-7.32 ( m, 1H), 7.27-7.25 (m, 2H), 5.13 (d, 2H), 5.10-5.07 (m, 4H), 3.96 (s, 2H), 3.91 (s, 2H), 3.70 (s, 2H), 2.96 (s, 3H). |
| 204 | 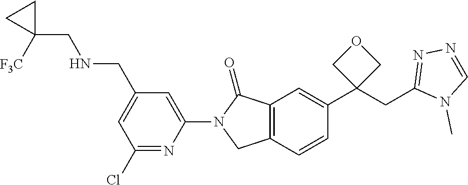 [SMILES: Cn1cnnc1CC1(c2ccc3c(c2)C(=O)N(c2cc(CNCC4(C(F)(F)F)CC4)cc(Cl)n2)C3)COC1] Starting materials: Int-1 and Int-198 Reaction conditions: A″ | LCMS: 547.2 [M + H]⁺; ¹HNMR (400 MHz, CD3OD ): δ 8.51 (s, 1H), 8.20 (s, 1H), 7.62 (d, 1H), 7.49 (s, 1H), 7.26 - 7.24 (m, 2H), 5.13 (d, 2H), 5.10- 5.07 (m, 4H), 3.87 (s, 2H), 3.70 (s, 2H), 2.96 (s, 3H), 2.84 (s, 2H), 1.05- 0.98 (m, 2H), 0.88-0.83 (m, 2H). |
| 205 | 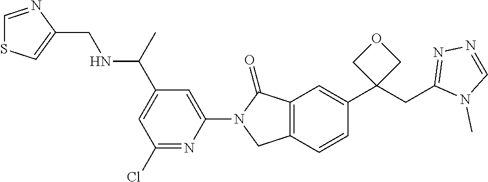 [SMILES: CC(NCc1cscn1)c1cc(Cl)nc(N2Cc3ccc(C4(Cc5nncn5C)COC4)cc3C2=O)c1] Starting materials: Int-1 and Int-199 Reaction conditions: A″ | LCMS: 536.3 [M + H]⁺; ¹HNMR (400 MHz, DMSO-d₆): δ 9.04 (s, 1H), 8.50 (s, 1H), 8.21 (s, 1H), 8.14 (s, 1H), 7.62 (d, 1H), 7.48 (s, 2H), 7.36 (dd, 1H), 7.32 (s, 1H), 5.03 (s, 2H), 4.98 (d, 2H), 4.91 (d, 2H), 3. 85 (q, 1H), 3.72 (s, 2H), 3.57 (s, 2H), 3.00 (s, 3H), 1.32 (d, 3H). |
| 206 | 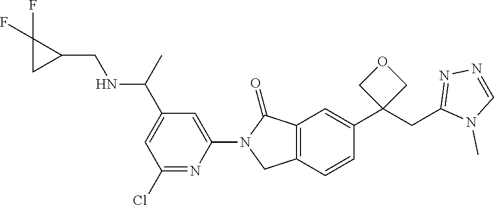 [SMILES: CC(NCC1CC1(F)F)c1cc(Cl)nc(N2Cc3ccc(C4(Cc5nncn5C)COC4)cc3C2=O)c1] Diastereomer 1 Starting materials: Int-1 and Int-194 Reaction conditions: A″ | LCMS: 529.3 [M + H]⁺; ¹HNMR (400 MHz, DMSO-d6): δ 8.48 (d, 1H), 8.21 (s, 1H), 7.62 (d, 1H), 7.49 (d, 1H), 7.36 (dd, 1H), 7.30 (d, 1H), 5.02 (s, 2H), 4.98 (d, 2H), 4.91 (d, 2H), 3.88-3.80 (m, 1H), 3.57 (s, 2H), 3.01 (s, 3H), 2.45-2.35 (m, 1H), 1.85-1.75 (m, 1H), 1.55-1.45 (m, 1H), 1.28 (d, 3H), 1.21-1.08 (m, 1H). |
| 207 | 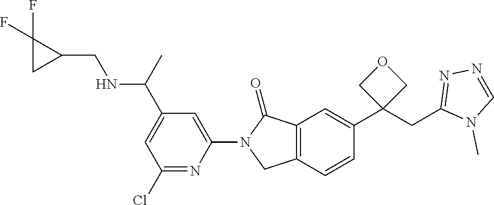 [SMILES: CC(NCC1CC1(F)F)c1cc(Cl)nc(N2Cc3ccc(C4(Cc5nncn5C)COC4)cc3C2=O)c1] Diastereomer 2 Starting materials: Int-1 and Int-194 Reaction conditions: A″ | LCMS: 529.4 [M + H]⁺; ¹H-NMR (400 MHz, DMSO-d6): δ 8.47 (s, 1H), 8.21 (s, 1H), 7.62 (d, 1H), 7.49 (s, 1H), 7.36 (dd, 1H), 7.31 (d, 1H), 5.02 (s, 2H), 4.98(d, 2H), 4.91 (d, 2H), 3.83- 3.78 (m, 1H), 3.57 (s, 2H), 3.01 (s, 3H), 2.42-2.37 (m, 1H), 1.85-1.75 (m, 1H), 1.55-1.45-(m, 1H), 1.29 (d, 3H), 1.20-1.10 (m, 1H). |
| 208 | 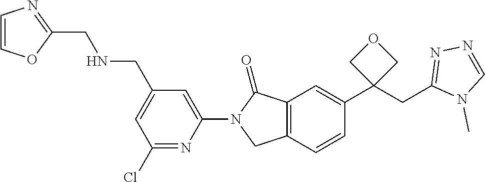 [SMILES: Cn1cnnc1CC1(c2ccc3c(c2)C(=O)N(c2cc(CNCc4ncco4)cc(Cl)n2)C3)COC1] Starting materials: Int-1 and Int-201 Reaction conditions: A″ | LCMS: 506.1 [M + H]⁺; ¹H-NMR (400 MHz, DMSO-d6): δ 8.46(s, 1H), 8.21 (s, 1H), 8.05 (s, 1H), 7.62 (d, 1H), 7.50 (s, 1H), 7.37 (dd, 1H), 7.25 (s, 1H), 7.15 (s, 1H), 5.03 (s, 2H), 4.98 (d, 2H), 4.91(d, 2H), 3.84 (s, 2H), 3.82 (s, 2H), 3.57 (s, 2H), 3.15-3.10 (m, 1H), 3.02 (s, 3H). |
| 209 | 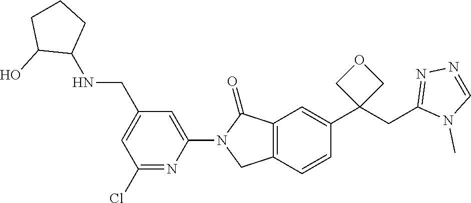 [SMILES: Cn1cnnc1CC1(c2ccc3c(c2)C(=O)N(c2cc(CNC4CCCC4O)cc(Cl)n2)C3)COC1] Diastereomer 1 Starting materials: Int-1 and Int-191 Reaction conditions: A″ | LCMS: 509.2 [M + H]⁺; ¹H-NMR (400 MHz, CD3OD ): δ 8.52 (s, 1H), 8.20 (s, 1H), 7.63 (d, 1H), 7.49 (s, 1H), 7.28-7.25 (m, 2H), , 5.13 (d, 2H), 5.11 (s, 2H), 5.08 (d, 2H), 3.98-3.95 (m, 1H), 3.91 (bs, 2H), 3.70 (s, 2H), 2.96 (s, 3H), 2.95-2.89 (m, 1H), 2.10 - 1.97 (m, 2H), 1.75-1.70 (m, 2H), 1.61-1.56 (m, 1H), 1.48-1.40 (m, 1H). |
| 210 | 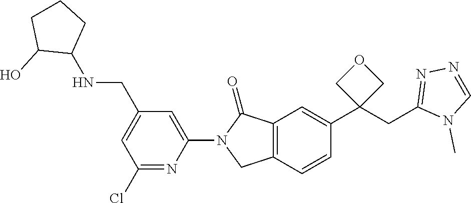 [SMILES: Cn1cnnc1CC1(c2ccc3c(c2)C(=O)N(c2cc(CNC4CCCC4O)cc(Cl)n2)C3)COC1] Diastereomer 2 Starting materials: Int-1 and Int-191 Reaction conditions: A″ | LCMS: 509.2 [M + H]⁺; ¹H-NMR (400 MHz, CD3OD ): δ 8.52 (s, 1H), 8.20 (s, 1H), 7.63 (d, 1H), 7.49 (s, 1H), 7.28-7.25 (m, 2H), 5.13 (d, 2H), 5.10 (s, 2H), 5.08 (d, 2H), 3.98-3.95 (m, 1H), 3.91 (bs, 2H), 3.70 (s, 2H), 2.96 (s, 3H), 2.95-2.89 (m, 1H), 2.10-1.98 (m, 2H), 1.75-1.70 (m, 2H), 1.61-1.56 (m, 1H), 1.50-1.44 (m, 1H). |
| 211 | 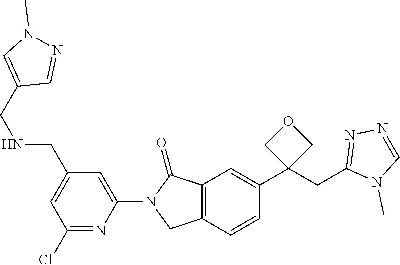 [SMILES: Cn1cc(CNCc2cc(Cl)nc(N3Cc4ccc(C5(Cc6nncn6C)COC5)cc4C3=O)c2)cn1] Starting materials: Int-1 and Int-202 Reaction conditions: A″ | LCMS: 521.1 [M + 2 + H]⁺; ¹HNMR (400 MHz, DMSO-d₆): δ 8.49 (s, 1H), 8.21 (s, 1H), 7.62 (d, 1H), 7.58 (s, 1H), 7.51 (s, 1H), 7.38-7.35 (m, 2H), 7.27 (s, 1H), 5.03 (s, 2H), 4.97 (d, 2H), 4.91 (d, 2H), 3.80 (s, 3H), 3.76 (s, 2H), 3.58 (s, 2H), 3.54 (s, 2H), 3.03 (s, 3H). |
| 212 | 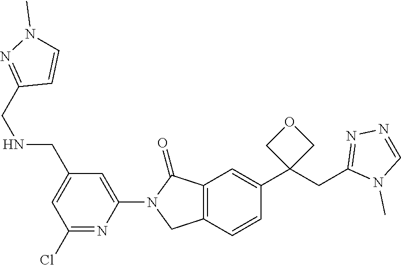 [SMILES: Cn1ccc(CNCc2cc(Cl)nc(N3Cc4ccc(C5(Cc6nncn6C)COC5)cc4C3=O)c2)n1] Starting materials: Int-1 and Int-208 Reaction conditions: A″ | LCMS: 519.2 [M + H]⁺; ¹H-NMR (400 MHz, DMSO-d6): δ 8.48 (s, 1H), 8.20 (s, 1H), 7.62 (s, 1H), 7.60 (s, 1H), 7.58 (d, 1H), 7.49 (d, 1H), 7.35 (dd, 2H), 7.25 (s, 1H), 6.19 (d, 1H), 5.02 (s, 2H), 4.96 (d, 2H), 4.89 (d, 2H), 3.77 (s, 5H), 3.61 (brs, 2H), 3.56 (s, 2H), 3.00 (s, 3H). |
| 213 | 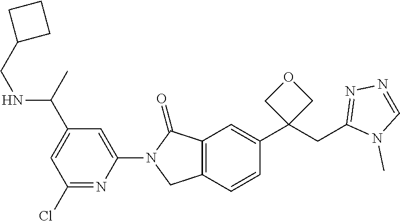 [SMILES: CC(NCC1CCC1)c1cc(Cl)nc(N2Cc3ccc(C4(Cc5nncn5C)COC4)cc3C2=O)c1] Enantiomer 1 Starting materials: Int-1 and Int-203 Reaction conditions: A″ | LCMS: 507.2 [M + H]⁺; ¹H-NMR (400 MHz, DMSO-d6): δ 8.48 (s, 1H), 8.21 (s, 1H), 7.61 (d, 1H), 7.49 (s, 1H), 7.35 (dd, 1H), 7.28 (s, 1H), 5.02 (s, 2H), 4.97 (d, 2H), 4.91 (d, 2H), 3.75 (q,1H), 3.57 (s, 2H), 3.01 (s, 3H), 2.49-2.41 (m, 1H), 2.39-2.31 (m, 1H), 2.30-2.28 (m, 1H), 1.99-1.95 (m, 2H), 1.83-1.76 (m, 2H), 1.64-1.57 (m, 2H), 1.25 (d, 3H). |
| 214 | 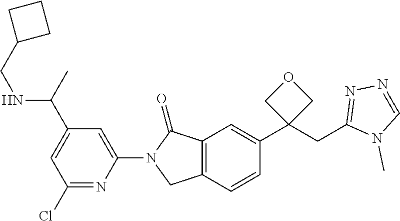 [SMILES: CC(NCC1CCC1)c1cc(Cl)nc(N2Cc3ccc(C4(Cc5nncn5C)COC4)cc3C2=O)c1] Enantiomer 2 Starting materials: Int-1 and Int-203 Reaction conditions: A″ | LCMS: 507.2 [M + H]⁺; ¹H-NMR (400 MHz, DMSO-d6): δ 8.48 (s, 1H), 8.21 (s, 1H), 7.62 (d, 1H), 7.49 (s, 1H), 7.35 (dd, 1H), 7.28 (d, 1H), 5.02 (s, 2H), 4.98 (d, 2H), 4.91 (d, 2H), 3.75 (q, 1H), 3.57 (s, 2H), 3.01 (s, 3H), 2.49- 2.41 (m, 1H), 2.39-2.28 (m, 2H), 1.99 - 1.95 (m, 2H), 1.83-1.76 (m, 2H), 1.64-1.57 (m, 2H), 1.25 (d, 3H). |
| 215 | 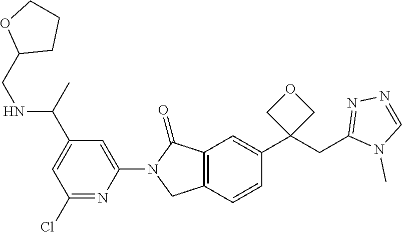 [SMILES: CC(NCC1CCCO1)c1cc(Cl)nc(N2Cc3ccc(C4(Cc5nncn5C)COC4)cc3C2=O)c1] Diastereomer 1 Starting materials: Int-1 and Int-205 Reaction conditions: B″ | LCMS: 524.2 [M + H]⁺; ¹HNMR (400 MHz, CD₃OD): δ 8.50 (s, 1H), 8.20 (s, 1H), 7.61 (d, 1H), 7.48 (s, 1H), 7.26- 7.24 (m, 2H), 5.12 (d, 2H), 5.09-5.07 (m, 4H), 4.02-3.99 (m, 1H), 3.91- 3.82 (m, 2H), 3.78-3.73 (m, 1H), 3.70 (s, 2H), 2.96 (s, 3H), 2.52-2.47 (m, 2H), 2.05-1.96 (m, 1H), 1.93-1.87 (m, 2H), 1.57-1.54 (m, 1H), 1.41 (d, 3H). |
| 216 | 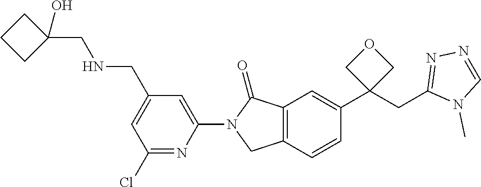 [SMILES: Cn1cnnc1CC1(c2ccc3c(c2)C(=O)N(c2cc(CNCC4(O)CCC4)cc(Cl)n2)C3)COC1] Starting materials: Int-1 and Int-207 Reaction conditions: C″ | LCMS: 509.3 [M + H]⁺; ¹H-NMR (400 MHz, DMSO-d6): δ 8.51 (s, 1H), 8.21 (s, 1H), 7.62 (d, 1H), 7.51 (s, 1H), 7.36 (dd, 1H), 7.28 (s, 1H), 5.03 (s, 2H), 4.97 (d, 2H), 4.95 (s, 1H), 4.91 (d, 2H), 3.86 (s, 2H), 3.57 (s, 2H), 3.02 (s, 3H), 2.55 (s, 2H), 2.10-2.00 (m, 2H), 1.96-1.85 (m, 2H). 1.69-1.55 (m, 1H), 1.48-1.36 (m, 1H). |
| 217 | 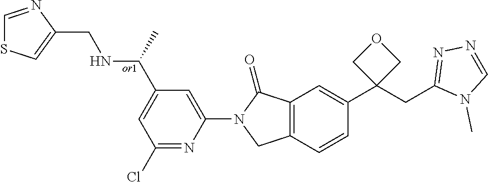 [SMILES: C[C@@H](NCc1cscn1)c1cc(Cl)nc(N2Cc3ccc(C4(Cc5nncn5C)COC4)cc3C2=O)c1] Enantiomer 1 Starting materials: Int-1 and Int-199 Reaction conditions: C″ | LCMS: 536.2 [M + H]⁺; ¹H-NMR (400 MHz, DMSO-d₆): δ 9.04 (d, 1H), 8.50 (s, 1H), 8.21 (s, 1H), 7.62 (d, 1H), 7.48 (s, 2H), 7.36 (dd, 1H), 7.32 (s, 1H), 5.03 (s, 2H), 4.98 (d, 2H), 4.91 (d, 2H), 3.89-3.81 (m, 1H), 3.72 (d, 2H), 3.57 (s, 2H), 3.00 (s, 3H), 2.85-2.78 (m, 1H), 1.32 (d, 3H). |
| 218 | 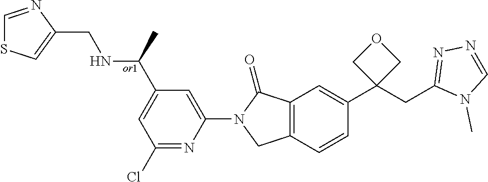 [SMILES: C[C@H](NCc1cscn1)c1cc(Cl)nc(N2Cc3ccc(C4(Cc5nncn5C)COC4)cc3C2=O)c1] Enantiomer 2 Starting materials: Int-1 and Int-199 Reaction conditions: C″ | LCMS: 536.2 [M + H]⁺; ¹H-NMR (400 MHz, DMSO-d₆): δ 9.04 (d, 1H), 8.50 (s, 1H), 8.21 (s, 1H), 7.62 (d, 1H), 7.48 (s, 2H), 7.36 (dd, 1H), 7.32 (s, 1H), 5.03 (s, 2H), 4.98 (d, 2H), 4.91 (d, 2H), 3.89-3.81 (m, 1H), 3.71 (d, 2H), 3.57 (s, 2H), 3.00 (s, 3H), 2.85-2.78 (m, 1H), 1.31 (d, 3H). |
| 219 | 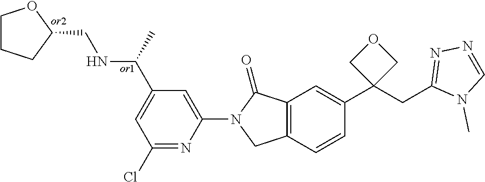 [SMILES: C[C@@H](NC[C@@H]1CCCO1)c1cc(Cl)nc(N2Cc3ccc(C4(Cc5nncn5C)COC4)cc3C2=O)c1] Enantiomer 1 Starting materials: Int-1 and Int-205 Reaction conditions: C″ | LCMS: 523.2 [M + H]⁺; ¹HNMR (400 MHz, DMSO-d₆): δ 8.47 (s, 1H), 8.21 (s, 1H), 7.62 (d, 1H), 7.49 (s, 1H), 7.35 (dd, 1H), 7.29 (d, 1H), 5.02 (s, 2H), 4.98 (d, 2H), 4.91 (d, 2H), 3.86-3.80 (m, 2H), 3.74-3.68 (m, 1H), 3.62- 3.58 (m, 1H), 3.57 (s, 2H), 3.01 (s, 3H), 2.48-2.43 (m, 1H), 2.40-2.35 (m, 1H), 2.20-2.10 (m, 1H), 1.91-1.75 (m, 3H), 1.59-1.50 (m, 1H), 1.28 (d, 3H). |
| 220 | 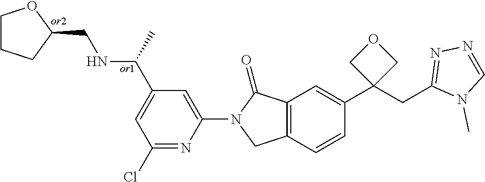 [SMILES: C[C@@H](NC[C@H]1CCCO1)c1cc(Cl)nc(N2Cc3ccc(C4(Cc5nncn5C)COC4)cc3C2=O)c1] Enantiomer 2 Starting materials: Int-1 and Int-205 Reaction conditions: C″ | LCMS: 523.2 [M + H]⁺; ¹HNMR (400 MHz, DMSO-d₆): δ 8.47 (s, 1H), 8.21 (s, 1H), 7.62 (d, 1H), 7.48 (s, 1H), 7.35 (dd, 1H), 7.29 (d, 1H), 5.02 (s, 2H), 4.98 (d, 2H), 4.91(d, 2H), 3.86-3.80 (m, 2H), 3.74-3.70 (m, 1H), 3.62-3.58 (m, 1H), 3.57 (s, 2H), 3.01 (s, 3H), 2.48-2.43 (m, 1H), 2.40-2.35 (m, 1H), 2.20-2.10 (m, 1H), 1.90-1.76 (m, 3H), 1.60-1.54 (m, 1H), 1.28 (d, 3H). |
| 221 | 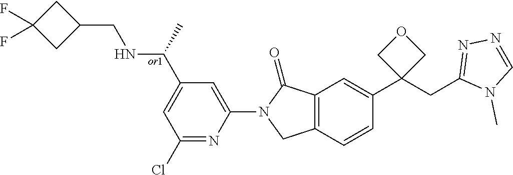 [SMILES: C[C@@H](NCC1CC(F)(F)C1)c1cc(Cl)nc(N2Cc3ccc(C4(Cc5nncn5C)COC4)cc3C2=O)c1] Enantiomer 1 Starting materials: Int-1 and Int-210 Reaction conditions: C″ | LCMS: 543.2 [M + H]⁺; ¹H-NMR (400 MHz, DMSO-d₆): δ 8.50 (s, 1H), 8.21 (s, 1H), 7.62 (d, 1H), 7.50 (s, 1H), 7.36 (dd, 1H), 7.28 (s, 1H), 5.02 (s, 2H), 4.98 (d, 2H), 4.91 (d, 2H) , 3.82-3.74 (m, 1H), 3.57 (s, 2H), 3.01 (s, 3H), 2.65-2.55 (m, 3H), 2.45-2.35 (m, 1H), 2.30-2.15 (m, 3H), 1.27 (d, 3H). |
| 222 | 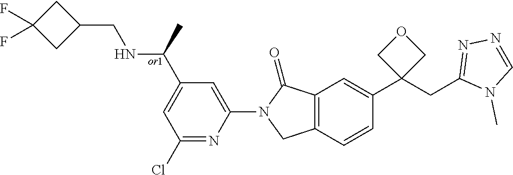 [SMILES: C[C@H](NCC1CC(F)(F)C1)c1cc(Cl)nc(N2Cc3ccc(C4(Cc5nncn5C)COC4)cc3C2=O)c1] Enantiomer 2 Starting materials: Int-1 and Int-210 Reaction conditions: C″ | LCMS: 543.2 [M + H]⁺; ¹H-NMR (400 MHz, DMSO-d₆): δ 8.50 (s, 1H), 8.21 (s, 1H), 7.62 (d, 1H), 7.50 (s, 1H), 7.36 (dd, 1H), 7.28 (s, 1H),5.02 (s, 2H), 4.98 (d, 2H), 4.91 (d, 2H), 3.80-3.75 (m, 1H), 3.57 (s, 2H), 3.02 (s, 3H), 2.65-2.55 (m, 3H), 2.45-2.35 (m, 1H), 2.32-2.14 (m, 3H), 1.27 (d, 3H). |
| 223 | 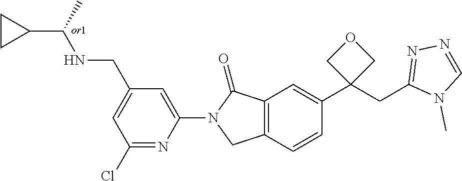 [SMILES: C[C@H](NCc1cc(Cl)nc(N2Cc3ccc(C4(Cc5nncn5C)COC4)cc3C2=O)c1)C1CC1] Enantiomer 1 Starting materials: Int-1 and Int-206 Reaction conditions: D″ | LCMS: 493.3 [M + H]⁺; ¹H-NMR (400 MHz, DMSO-d6): δ 8.49 (d, 1H), 8.21 (s, 1H), 7.62 (dd, 1H), 7.51 (d, 1H), 7.36 (dd, 1H), 7.28 (d, 1H), 5.02 (s, 2H), 5.00 (d, 2H), 4.91 (d, 2H), 3.87 (d, 2H), 3.57 (s, 2H), 3.03 (s, 3H), 1.92 - 1.80 (m, 1H), 1.29-1.23 (m, 1H), 1.09 (d, 3H), 0.72-0.61 (m, 1H), 0.49 - 0.40 (m, 1H), 0.39-0.30 (m, 1H), 0.26-0.16 (m, 1H). |
| 224 | 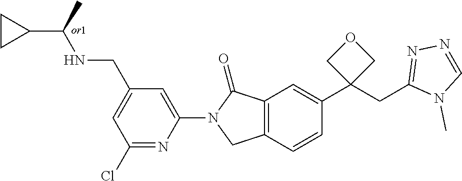 [SMILES: C[C@@H](NCc1cc(Cl)nc(N2Cc3ccc(C4(Cc5nncn5C)COC4)cc3C2=O)c1)C1CC1] Enantiomer 2 Starting materials: Int-1 and Int-206 Reaction conditions: D″ | LCMS: 493.2 [M + H]⁺; ¹H-NMR (400 MHz, DMSO-d6): δ 8.49 (d, 1H), 8.21 (s, 1H), 7.62 (dd, 1H), 7.51(s, 1H), 7.36 (dd, 1H), 7.28 (d, 1H), 5.02 (s, 2H), 4.97 (d, 2H), 4.90 (d, 2H) , 3.87 (d, 2H), 3.57 (s, 2H), 3.03 (s, 3H), 1.92 - 1.82 (m, 1H), 1.30-1.20 (m, 1H), 1.10 (d, 3H), 0.71-0.61 (m, 1H), 0.49 0.40 (m, 1H), 0.38-0.28 (m,1H), 0.27-0.18 (m, 1H). |
| 225 | 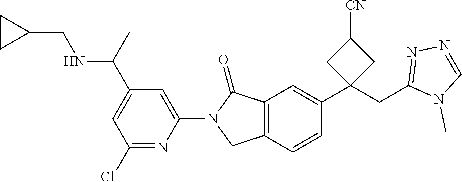 [SMILES: CC(NCC1CC1)c1cc(Cl)nc(N2Cc3ccc(C4(Cc5nncn5C)CC(C#N)C4)cc3C2=O)c1] Starting materials: Int-23 and Int-103 Reaction conditions: A″ | LCMS: 516.3 [M + H]⁺; ¹H-NMR (400 MHz, DMSO-d6): δ 8.48 (s, 1H), 8.19 (s, 1H), 7.62 (d, 1H), 7.49 ( s, 1H), 7.39 (dd, 1H), 7.29 (d, 1H), 5.02 (s, 2H), 3.90-3.80 (m, 1H), 3.36 (s, 2H), 3.26-3.19 (m, 2H), 2.98-2.88 (m, 3H), 2.85 (s, 3H), 2.35-2.28 (m, 1H), 2.25-2.20 (m, 1H), 1.28 (d, 3H), 0.92-0.80 (m, 1H), 0.46-0.31 (m, 2H), 0.08-0.06 (m, 2H). |
| 226 | 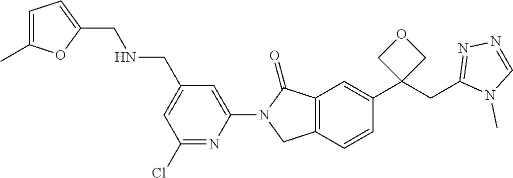 [SMILES: Cc1ccc(CNCc2cc(Cl)nc(N3Cc4ccc(C5(Cc6nncn6C)COC5)cc4C3=O)c2)o1] Starting materials: Int-1 and Int-211 Reaction conditions: A″ | LCMS: 519.0 [M + H]⁺; ¹H-NMR (400 MHz, DMSO-d6): δ 8.45 (d, 1H), 8.21 (s, 1H), 7.62 (d, 1H), 7.50 ( s, 1H), 7.36 (dd, 1H), 7.25 (s, 1H), 6.12 (d, 1H), 5.97 (dd, 1H), 5.02 (s, 2H), 4.98 (d, 2H), 4.91 (d, 2H), 3.77 (s, 2H), 3.62 (s, 2H), 3.57 (s, 2H), 3.03 (s, 3H), 2.15 (s, 3H). |
| 227 | 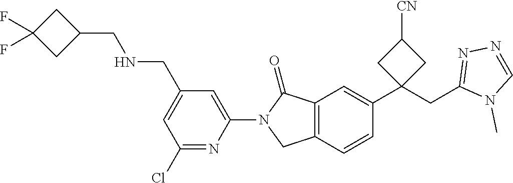 [SMILES: Cn1cnnc1CC1(c2ccc3c(c2)C(=O)N(c2cc(CNCC4CC(F)(F)C4)cc(Cl)n2)C3)CC(C#N)C1] Starting materials: Int-23 and Int-182 Reaction conditions: A″ | LCMS: 552.3 [M + H]⁺; ¹H-NMR (400 MHz, DMSO-d6): δ 8.49 (d, 1H), 8.19 (s, 1H), 7.62 (d, 1H), 7.52 ( s, 1H), 7.42-7.37 (m, 1H), 7.26 (d, 1H), 5.03 (s, 2H), 3.79 (s, 2H), 3.36 (s, 2H), 3.28 - 3.18 (m, 1H), 2.95-2.88 (m, 3H), 2.88 (s, 3H), 2.66-2.53 (m, 5H), 2.38 - 2.30 (m, 2H). |
| 228 | 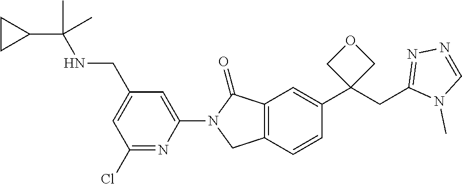 [SMILES: Cn1cnnc1CC1(c2ccc3c(c2)C(=O)N(c2cc(CNC(C)(C)C4CC4)cc(Cl)n2)C3)COC1] Starting materials: Int-1 and Int-212 Reaction conditions: A″ | LCMS: 507.2 [M + H]⁺; ¹H-NMR (400 MHz, DMSO-d6): 8 8.53 (s, 1H), 8.21 (s, 1H), 7.62 (d, 1H), 7.51 ( s, 1H), 7.37 (dd, 1H), 7.28 (s, 1H), 5.02 (s, 2H), 4.98 (d, 2H), 4.91 (d, 2H), 3.84 (s, 2H), 3.57 (s, 2H), 3.03 (s, 3H), 0.95 (s, 6H), 0.90-0.80 ( m, 1H), 0.31-0.28 (m, 4H). |
| 229 | 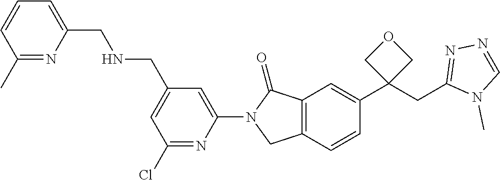 [SMILES: Cc1cccc(CNCc2cc(Cl)nc(N3Cc4ccc(C5(Cc6nncn6C)COC5)cc4C3=O)c2)n1] Starting materials: Int-1 and Int-213 Reaction conditions: C″ | LCMS: 530.2 [M + H]⁺; ¹H-NMR (400 MHz, DMSO-d6): δ 8.51 (s, 1H), 8.21 (s, 1H), 7.65-7.61 (m, 2H), 7.50 (s, 1H), 7.36 (dd, 1H), 7.29-7.28 (m, 2H), 7.12 (d, 1H), 5.03 (s, 2H), 4.97 (d, 2H), 4.91 (d, 2H), 3.83 (s, 2H), 3.76 (s, 2H), 3.57 (s, 2H), 3.02 (s, 3H), 2.44 (s, 3H). |
| 230 | 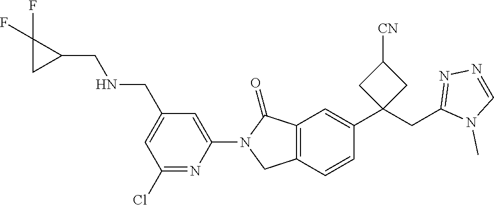 [SMILES: Cn1cnnc1CC1(c2ccc3c(c2)C(=O)N(c2cc(CNCC4CC4(F)F)cc(Cl)n2)C3)CC(C#N)C1] Starting materials: Int-23 and Int-184 Reaction conditions: D″ | LCMS: 538.2 [M + H]⁺; ¹H-NMR (400 MHz, DMSO-d6): δ 8.48 (s, 1H), 8.22-8.15 (m, 1H), 7.62 (d, 1H), 7.50 (d, 1H), 7.40 (dd, 1H), 7.28 (d, 1H), 5.03 (s, 2H), 3.88-3.78 ( m, 2H), 3.45 (s, 2H), 3.30-3.20 (m, 1H), 2.95 - 2.86 (m, 3H), 2.87 (s, 3H), 2.70- 2.59 (m, 2H), 1.92-1.80 (m, 1H), 1.60 - 1.50 (m, 1H), 1.28-1.15 (m, 1H). |
| 231 | 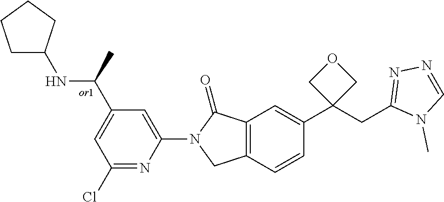 [SMILES: C[C@H](NC1CCCC1)c1cc(Cl)nc(N2Cc3ccc(C4(Cc5nncn5C)COC4)cc3C2=O)c1] Enantiomer 1 Starting materials: Int-1 and Int-214 Reaction conditions: A″ | LCMS: 507.20 [M + H]⁺; ¹HNMR (400 MHz, DMSO-d₆): δ 8.48 (s, 1H), 8.21 (s, 1H), 7.62 (d, 1H), 7.48 ( s, 1H), 7.35 (dd, 1H), 7.31 (s, 1H), 5.02 (s, 2H), 4.98 (d, 2H), 4.91 (d, 2H), 3.85- 3.78 (m, 1H), 3.57 (s, 2H), 3.01 (s, 3H), 2.84-2.75 (m, 1H), 1.71-1.51 (m, 4H), 1.48-1.30 (m, 4H), 1.29- 1.20 (m, 3H). |
| 232 | 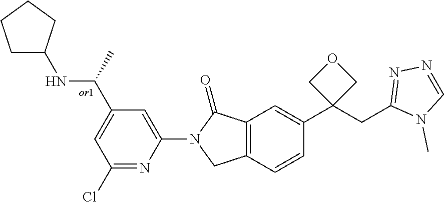 [SMILES: C[C@@H](NC1CCCC1)c1cc(Cl)nc(N2Cc3ccc(C4(Cc5nncn5C)COC4)cc3C2=O)c1] Enantiomer 2 Starting materials: Int-1 and Int-214 Reaction conditions: A″ | LCMS: 507.2 [M + H]⁺; ¹HNMR (400 MHz, DMSO-d₆): δ 8.48 (d, 1H), 8.21 (s, 1H), 7.62 (d, 1H), 7.48 ( s, 1H), 7.36 (d, 1H), 7.31 (d, 1H), 5.02 (s, 2H), 4.98 (d, 2H), 4.91 (d, 2H), 3.82-3.79 (m, 1H), 3.57 (s, 2H), 3.01 (s, 3H), 2.81-2.76 (m, 1H), 1.71-1.55 (m, 4H), 1.47-1.30 (m, 4H), 1.30-1.20 (m, 3H). |
| 233 | 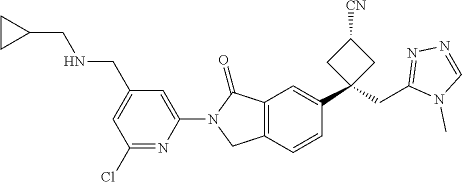 [SMILES: Cn1cnnc1C[C@]1(c2ccc3c(c2)C(=O)N(c2cc(CNCC4CC4)cc(Cl)n2)C3)C[C@H](C#N)C1] Diastereomer 1 Starting materials: Int-23 and Int-76 Reaction conditions: A″ | LCMS: 502.3 [M + H]⁺; ¹HNMR (400 MHz, DMSO-d6): δ 8.48 (d, 1H), 8.17 (s, 1H), 7.59 (d, 1H), 7.35 (d, 1H), 7.26 (s, 1H), 7.21 (dd, 1H), 5.02 (s, 2H), 3.81 (s, 2H), 3.80-3.75 (m, 1H), 3.32 (s, 3H), 3.11-3.08 (m, 2H), 2.76 (s, 3H), 2.68-2.71 (m, 1H), 2.36 ( d, 2H), 0.96-0.82 (m, 1H), 0.42-0.39 (m, 2H), 0.12-0.05 (m, 2H). |
| 234 | 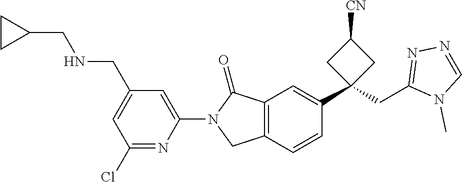 [SMILES: Cn1cnnc1C[C@]1(c2ccc3c(c2)C(=O)N(c2cc(CNCC4CC4)cc(Cl)n2)C3)C[C@@H](C#N)C1] Diastereomer 2 Starting materials: Int-23 and Int-76 Reaction conditions: A″ | LCMS: 502.3 [M + H]⁺; ¹HNMR (400 MHz, DMSO-d6): δ 8.48 (d, 1H), 8.19 (s, 1H), 7.61 (d, 1H), 7.50 ( s, 1H), 7.38 (d, 1H), 7.26 (d, 1H), 5.02 (s, 2H), 3.81 (s, 2H), 3.36 (s, 2H), 3.30-3.24 (m, 1H), 2.91-2.89 (m, 4H), 2.87 (s, 3H), 2.38 (d, 2H), 0.95-0.82 (m, 1H), 0.43-0.38 (m, 2H), 0.13-0.10 (m, 2H). |
| 235 | 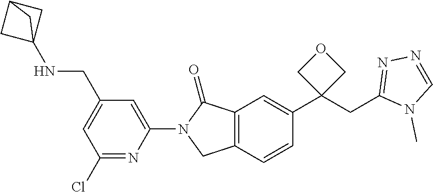 [SMILES: Cn1cnnc1CC1(c2ccc3c(c2)C(=O)N(c2cc(CNC45CC(C4)C5)cc(Cl)n2)C3)COC1] Starting materials: Int-1 and Int-215 Reaction conditions: A″ | LCMS: 491.3 [M + H]⁺; ¹HNMR (400 MHz, DMSO-d6): δ 8.49 (d, 1H), 8.21 (s, 1H), 7.61 (d, 1H), 7.50 (s, 1H), 7.38 (1H, d), 7.26 (d, 1H), 5.03 (s, 2H), 4.97 (d, 2H), 4.91 (d, 2H), 3.78 (s, 2H), 3.57 (s, 2H), 3.03 (s, 3H), 2.32 (s, 1H), 1.70 (s, 6H). |
| 236 | 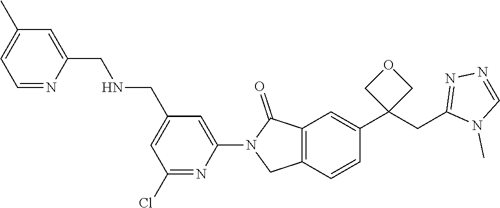 [SMILES: Cc1ccnc(CNCc2cc(Cl)nc(N3Cc4ccc(C5(Cc6nncn6C)COC5)cc4C3=O)c2)c1] Starting materials: Int-1 and Int-216 Reaction conditions: A″ | LCMS: 530.3 [M + H]⁺; ¹HNMR (400 MHz, DMSO-d6): δ 8.50, (d, 1H), 8.34 (d, 1H), 8.21 (s, 1H), 7.62 (d, 1H), 7.50 (d, 1H), 7.36 (dd, 1H,), 7.29 (d, 2H) 7.08 (d,1H), 5.03 (s, 2H), 4.97 (d, 2H), 4.90 (d, 2H), 3.83 (s, 2H), 3.77 (s, 2H), 3.57 (s, 2H), 3.02 (s, 3H), 2.32 (s, 3H). |
| 237 | 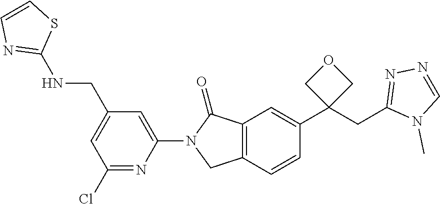 [SMILES: Cn1cnnc1CC1(c2ccc3c(c2)C(=O)N(c2cc(CNc4nccs4)cc(Cl)n2)C3)COC1] Starting materials: Int-1 and Int-217 Reaction conditions: A″ | LCMS: 508.1 [M + H]⁺; ¹HNMR (400 MHz, DMSO-d₆): δ 8.49 (d, 1H), 8.26 ( dd, 1H), 8.21 (s, 1H), 7.61 (d, 1H), 7.49 (d, 1H), 7.36 (dd, 1H), 7.21 ( s, 1H), 7.00 (d, 1H), 6.67 (d, 1H), 5.02 (s, 2H), 4.97 (d, 2H), 4.90 ( d, 2H), 4.54 (d, 2H), 3.57 (s, 2H), 3.01 (s, 3H). |
| 238 | 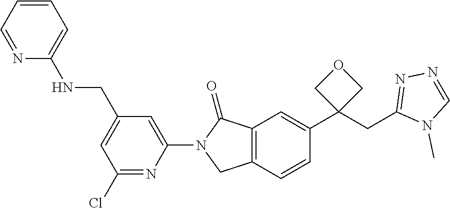 [SMILES: Cn1cnnc1CC1(c2ccc3c(c2)C(=O)N(c2cc(CNc4ccccn4)cc(Cl)n2)C3)COC1] Starting materials: Int-1 and Int-218 Reaction conditions: C″ | LCMS: 502.2 [M + H]⁺; ¹H-NMR (400 MHz, DMSO-d₆): δ 8.47 (d, 1H), 8.20 (s, 1H), 7.94-7.92 (m, 1H), 7.61 (d, 1H), 7.48 (s, 1H), 7.45-7.38 (m, 1H), 7.38-7.32 (m, 1H), 7.30-7.25 (m, 1H), 7.18 (d, 1H), 6.57 (d, 1H), 6.55- 6.50 (m, 1H), 5.00 (s, 2H), 4.97 (d, 2H), 4.90 (d, 2H), 4.55 (d, 2H), 3.56 (s, 2H), 3.00 (s, 3H). |
| 239 | 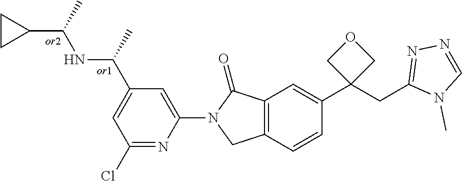 [SMILES: C[C@H](N[C@H](C)c1cc(Cl)nc(N2Cc3ccc(C4(Cc5nncn5C)COC4)cc3C2=O)c1)C1CC1] Enantiomer 1 Starting materials: Int-1 and Int-219 Reaction conditions: A″ | LCMS: 507.2 [M + H]⁺; ¹HNMR (400 MHz, DMSO-d₆): δ 8.46 (d, 1H), 8.21 (s, 1H), 7.61 (d, 1H), 7.50 ( d, 1H), 7.35 (dd, 1H), 7.32 (d, 1H), 5.07-4.96 (m, 4H), 4.95-4.88 (m, 2H), 3.94- 3.92 (m, 1H), 3.57 (s, 2H), 3.01 (s, 3H), 1.67-1.65 (m, 1H),, 1.27 (d, 3H), 1.03 (d, 3H), 0.68-0.61 (m, 1H), 0.31 - 0.29 (m, 2H), 0.18-0.12 (m, 1H). |
| 240 | 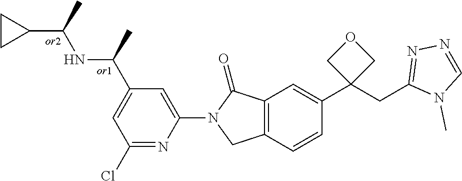 [SMILES: C[C@H](N[C@H](C)C1CC1)c1cc(Cl)nc(N2Cc3ccc(C4(Cc5nncn5C)COC4)cc3C2=O)c1] Enantiomer 2 Starting materials: Int-1 and Int-219 Reaction conditions: A″ | LCMS: 507.2 [M + H]⁺; ¹HNMR (400 MHz, DMSO-d₆): δ 8.46 (d, 1H), 8.22 (s, 1H), 7.61 (d, 1H), 7.50 (d, 1H), 7.36 - 7.33 (m, 2H), 5.01 (d, 2H), 4.99- 4.96 (m, 2H), 4.92-4.88 (m, 2H), 3.93 - 3.88 (m, 1H), 3.57 (s, 2H), 3.01 (s, 3H), 1.67-1.60 (m, 1H), 1.26 (d, 3H), 1.04 (d, 3H), 0.68-0.60 (m, 1H), 0.33 - 0.25 (m, 2H), 0.19-0.11 (s, 1H). |
| 241 | 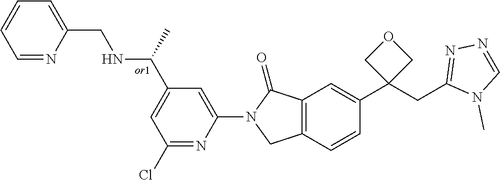 [SMILES: C[C@@H](NCc1ccccn1)c1cc(Cl)nc(N2Cc3ccc(C4(Cc5nncn5C)COC4)cc3C2=O)c1] Enantiomer 1 Starting materials: Int-1 and Int-220 Reaction conditions: A″ | LCMS: 530.2 [M + H]⁺ ; ¹HNMR (400 MHz, DMSO-d₆): δ 8.51 (s, 1H), 8.48 8.46 (m, 1H), 8.21 (s, 1H), 7.78-7.74 (m, 1H), 7.62 (d, 1H), 7.48-7.45 (m, 2H), 7.25 (dd, 1H), 7.24 (s, 1H), 7.25- 7.23 (m, 1H) 5.03 (s, 2H), 4.98 (d, 2H), 4.91 (d, 2H), 3.88-3.80 ( m, 1H), 3.71 - 3.61 (m, 2H), 3.57 (s, 2H), 3.00 (s, 3H), 2.97-2.94 (m, 1H), 1.33 (d, 3H). |
| 242 | 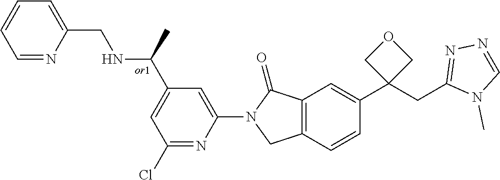 [SMILES: C[C@H](NCc1ccccn1)c1cc(Cl)nc(N2Cc3ccc(C4(Cc5nncn5C)COC4)cc3C2=O)c1] Enantiomer 2 Starting materials: Int-1 and Int-220 Reaction conditions: A″ | LCMS: 530.2 [M + H]⁺; ¹HNMR (400 MHz, DMSO-d₆): δ 8.51 (s, 1H), 8.48 - 8.46 ( d, 1H), 8.21 (s, 1H), 7.78- 7.74 (m, 1H), 7.62 (d, 1H), 7.46-7.47 (m, 2H), 7.36 (dd, 1H), 7.32 (d, 1H), 7.26-7.23 (m, 1H), 5.03 (s, 2H), 4.98 (d, 2H), 4.91 (d, 2H), 3.89-3.80 (m, 1H), 3.70-3.65 (m, 2H), 3.00 (s, 3H), 2.95-2.90 (m, 1H), 1.33 (d, 3H). |
| 243 | 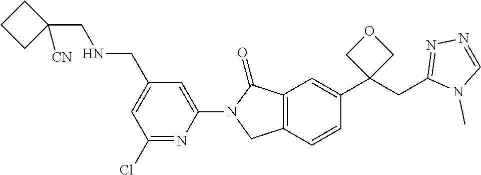 [SMILES: Cn1cnnc1CC1(c2ccc3c(c2)C(=O)N(c2cc(CNCC4(C#N)CCC4)cc(Cl)n2)C3)COC1] Starting materials: Int-1 and Int-221 Reaction conditions: A″ | LCMS: 518.2 [M + H]⁺; ¹HNMR (400 MHz, DMSO-d6): δ 8.49 (d, 1H), 8.21 (s, 1H), 7.62 (d,1H), 7.52 (s,1H), 7.36 (dd, 1H), 7.32 (d, 1H), 5.03 (s, 2H), 4.98 (d, 2H), 4.91 (d, 2H), 3.88 (bs,2H), 3.57 (s, 2H), 3.03 (s, 3H), 2.82 (s, 2H), 2.39-2.31 (m, 2H), 2.22- 2.12 (m, 2H), 2.05-1.95 (m, 2H). |
| 244 | 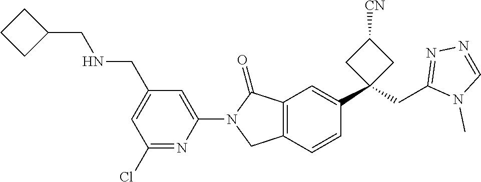 [SMILES: Cn1cnnc1C[C@]1(c2ccc3c(c2)C(=O)N(c2cc(CNCC4CCC4)cc(Cl)n2)C3)C[C@H](C#N)C1] Diastereomer 1 Starting materials: Int-23 and Int-172 Reaction conditions: D″ | LCMS: 516.2 [M + H]⁺; ¹HNMR (400 MHz, DMSO-d6): δ 8.48 (d, 1H), 8.17 (s, 1H), 7.59 (d, 1H), 7.35 ( s, 1H), 7.25 (d, 1H), 7.19 (dd, 1H), 5.02 (s, 2H), 3.81-3.78 (m, 1H), 3.76 (s, 2H), 3.29 (s, 2H), 3.12-3.02 (m, 2H), 2.76 (s, 3H), 2.65-2.59 (m, 2H), 2.46- 2.38 (m, 1H), 2.05-1.93 (m, 2H), 1.90 - 1.72 (m, 2H), 1.70-1.60 (m, 2H). |
| 245 | 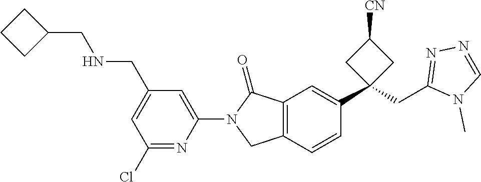 [SMILES: Cn1cnnc1C[C@]1(c2ccc3c(c2)C(=O)N(c2cc(CNCC4CCC4)cc(Cl)n2)C3)C[C@@H](C#N)C1] Diastereomer 2 Starting materials: Int-23 and Int-172 Reaction conditions: D″ | LCMS: 516.2 [M + H]⁺; ¹HNMR (400 MHz, DMSO-d6): δ 8.48 (d, 1H), 8.19 (s, 1H), 7.61 (d, 1H), 7.51 (d, 1H), 7.39 (dd, 1H), 7.25 (d, 1H), 5.02 (s, 2H), 3.76 (s, 2H), 3.36 (s, 2H), 3.29- 3.20 (m, 1H), 2.94-2.86 (m, 4H), 2.87 (s, 3H), 2.45-2.36 (m, 1H), 2.05- 1.95 (m, 2H), 1.90-1.73 (m, 2H), 1.72 - 1.60 (m, 2H). |
| 246 | 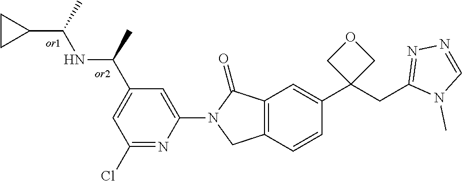 [SMILES: C[C@H](N[C@@H](C)C1CC1)c1cc(Cl)nc(N2Cc3ccc(C4(Cc5nncn5C)COC4)cc3C2=O)c1] Diastereomer 1 Starting materials: Int-1 and Int-219 Reaction conditions: A″ | LCMS: 507.2 [M + H]⁺; ¹HNMR (400 MHz, CD3OD): δ 8.48 (d, 1H), 8.20 (s, 1H), 7.62 ( d, 1H), 7.48 (d, 1H), 7.27- 7.24 (m, 2H), 5.13 (d, 2H), 5.10 (s, 2H), 5.08 (d, 2H), 4.32-4.26 (m, 1H), 3.70 (s, 2H), 2.95 (s, 3H), 1.81-1.77 (m, 1H), 1.39 (d, 3H), 1.17 (d, 3H), 0.72-0.64 (m, 1H), 0.63-0.55 (m,1H), 0.42-0.34 (m, 1H), 0.16- 0.12 (m,1H),-0.06-−0.10 (m, 1H). |
| 247 | 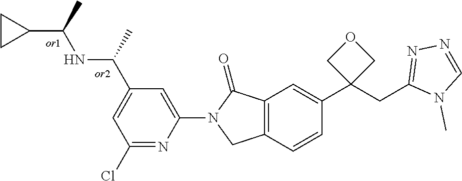 [SMILES: C[C@@H](N[C@H](C)C1CC1)c1cc(Cl)nc(N2Cc3ccc(C4(Cc5nncn5C)COC4)cc3C2=O)c1] Diastereomer 2 Starting materials: Int-1 and Int-219 Reaction conditions: A″ | LCMS: 507.2 [M + 1H]⁺; ¹HNMR (400 MHz, CD3OD): δ 8.49 (d, 1H), 8.20 (s, 1H), 7.63 (d, 1H), 7.48 (s, 1H), 7.27- 7.25 (m, 2H), 5.13 (d, 2H), 5.10 (s, 2H), 5.08 (d, 2H), 4.34-4.28 (m, 1H), 3.70 (s, 2H), 2.96 (s, 3H), 1.85-1.78 (m, 1H), 1.40 (d, 3H), 1.19 (d, 3H), 0.72-0.64 (m, 1H), 0.63-0.55 (m,1H), 0.45-0.34 (m, 1H), 0.18- 0.10 (m,1H),-0.04-−0.10 (m, 1H). |
| 248 | 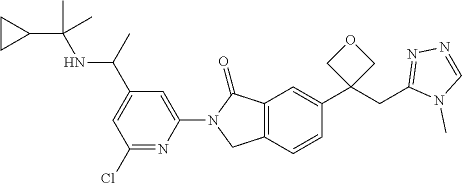 [SMILES: CC(NC(C)(C)C1CC1)c1cc(Cl)nc(N2Cc3ccc(C4(Cc5nncn5C)COC4)cc3C2=O)c1] Starting materials: Int-1 and Int-222 Reaction conditions: A″ | LCMS: 521.2 [M + H]⁺; ¹HNMR (400 MHz, DMSO-d₆): δ 8.60 (s, 1H), 8.21 (s, 1H), 7.61 (d, 1H), 7.49 (s, 1H), 7.36 - 7.33 (m , 2H), 5.01 (s, 2H), 4.99 (d, 2H), 4.97 (d, 2H), 4.17-4.09 ( m, 1H), 3.57 (s, 2H), 3.01 (s, 3H), 1.25 (d, 3H), 0.86 (s, 3H), 0.83 (s, 3H), 0.76-0.70 (m, 1H), 0.24-0.22 (m, 3H), 0.16- 0.09 (m, 1H). |
| 249 | 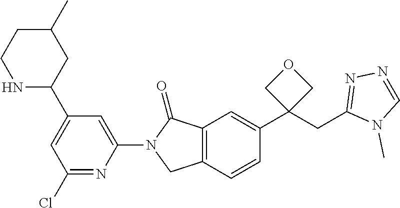 [SMILES: CC1CCNC(c2cc(Cl)nc(N3Cc4ccc(C5(Cc6nncn6C)COC5)cc4C3=O)c2)C1] Starting materials: Int-1 and Int-161 Reaction conditions: A″ | LCMS: 493.1 [M + H]⁺;¹HNMR (400 MHz, DMSO-d6): δ 8.50 (s, 1H), 8.21 (s, 1H), 7.62 (d, 1H), 7.47 ( s, 1H), 7.38 (dd, 1H), 7.27 (s, 1H), 5.02 (s, 2H), 4.98 (d, 2H), 4.91 (d, 2H), 3.66- 3.63 (m, 1H), 3.58 (s, 2H), 3.08-3.05 (m, 1H), 3.02 (s, 3H), 2.67-2.61 (m, 1H), 1.78-1.75 (m, 1H), 1.62-1.58 (m, 2H), 1.18-1.05 (m, 2H), 0.91 (d, 3H). |
| 250 | 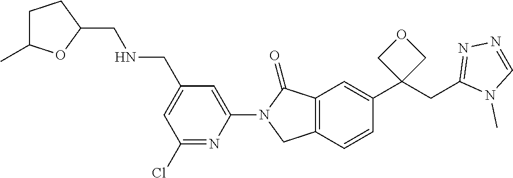 [SMILES: CC1CCC(CNCc2cc(Cl)nc(N3Cc4ccc(C5(Cc6nncn6C)COC5)cc4C3=O)c2)O1] Starting materials: Int-1 and Int-162 Reaction conditions: A″ | LCMS: 523.20 [M + H]⁺; ¹HNMR (400 MHz, DMSO-d6): δ 8.53 (d, 1H), 8.20 (s, 1H), 7.64 (dd, 1H), 7.49 ( s, 1H), 7.27-7.25 (m, 2H), 5.13 (d, 2H), 5.11 (s, 2H), 5.08 (d, 2H), 4.10-4.00 (m, 2H), 3.94 (s, 2H), 3.70 (s, 2H), 2.96 (s, 3H), 2.78 (dd, 1H), 2.70 (dd, 1H), 2.10 - 2.01 (m, 2H), 1.75-1.68 (m, 1H), 1.45-1.55 (m, 1H), 1.26 (d, 3H). |
| 251 | 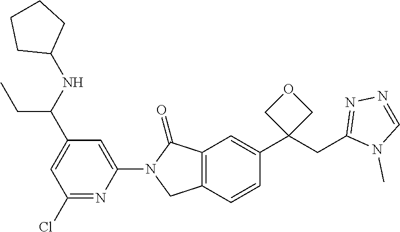 [SMILES: CCC(NC1CCCC1)c1cc(Cl)nc(N2Cc3ccc(C4(Cc5nncn5C)COC4)cc3C2=O)c1] Starting materials: Int-1 and Int-223 Reaction conditions: C″ | LCMS: 521.3 [M + H]⁺; ¹HNMR (400 MHz, DMSO-d₆): δ 8.44 (d, 1H), 8.21 (s, 1H), 7.62 (d, 1H), 7.47 (d, 1H), 7.35 (dd, 1H), 7.30 (d, 1H), 5.03 (s, 2H), 4.98 (d, 2H), 4.90 (d, 2H), 3.58-3.50 (m, 3H), 3.00 (s, 3H), 2.80-2.72 (m, 1H), 1.71-1.48 (m, 7H), 1.45-1.28 (m, 3H), 0.80 (t, 3H). |
| 252 | 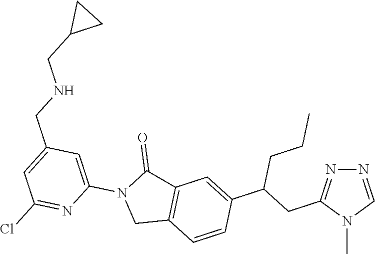 [SMILES: CCCC(Cc1nncn1C)c1ccc2c(c1)C(=O)N(c1cc(CNCC3CC3)cc(Cl)n1)C2] Starting materials: Int-121 and Int-76 Reaction conditions: A″ | LCMS: 479.2 [M + H]⁺; ¹H-NMR (400 MHz, DMSO-d₆): δ 8.50 (d, 1H), 8.25 (s, 1H), 7.67 (s, 1H), 7.63-7.57 (m, 2H), 7.26 (s, 1H), 5.01 (s, 2H), 3.81 (s, 2H), 3.46 (s, 3H), 3.35-3.22 (m, 1H), 3.12 (dd, 1H), 3.01 (dd 1H), 2.39 (d, 2H), 1.80-1.62 (m, 2H), 1.20-1.13 (m, 2H), 0.95-0.89 (m, 1H), 0.83 (t, 3H), 0.45-0.38 (m, 2H), 0.16-0.10 (m, 2H). |
| 253 | 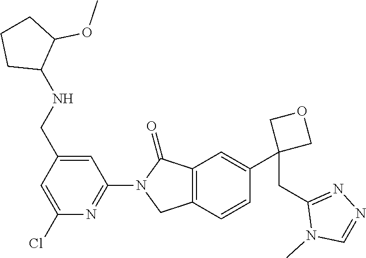 [SMILES: COC1CCCC1NCc1cc(Cl)nc(N2Cc3ccc(C4(Cc5nncn5C)COC4)cc3C2=O)c1] Starting materials: Int-1 and Int-224 Reaction conditions: A″ | LCMS: 523.3 [M + H]⁺; ¹H-NMR (400 MHz, DMSO-d₆): δ 8.49 (s, 1H), 8.21 (s, 1H), 7.62 (d, 1H), 7.51 ( d, 1H), 7.38 (dd, 1H) , 7.28 (s, 1H), 5.03 (s, 2H), 4.97 (d, 2H), 4.91 (d, 2H), 3.81 (s, 2H), 3.57 (s, 2H), 3.51-3.49 (m, 1H), 3.19 (s, 3H), 3.03 (s, 3H), 2.85-2.93 (m, 1H), 1.90-1.75 (m, 2H), 1.68- 1.48 (m, 3H), 1.40-1.31 (m, 1H). |
| 254 | 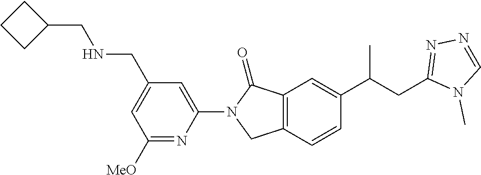 [SMILES: COc1cc(CNCC2CCC2)cc(N2Cc3ccc(C(C)Cc4nncn4C)cc3C2=O)n1] Starting materials: Int-2 and Int-225 Reaction conditions: A″ | LCMS: 461.3 [M + H]⁺; ¹HNMR (400 MHz, DMSO-d6): δ 8.28 (s, 1H), 8.08 (d, 1H), 7.71 (s, 1H), 7.63-7.59 (m, 2H), 6.58 (d, 1H), 5.08 (s, 2H), 3.91 (s, 3H), 3.71 (s, 2H), 3.49 (s, 3H), 3.48- 3.40 (m, 1H), 3.06 (d, 2H), 2.48-2.35 (m, 1H), 2.05-1.95 (m, 2H), 1.90-1.72 (m, 2H), 1.71-1.60 (m, 2H), 1.34 (d, 3H). |
| 255 | 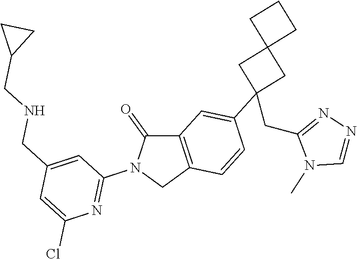 [SMILES: Cn1cnnc1CC1(c2ccc3c(c2)C(=O)N(c2cc(CNCC4CC4)cc(Cl)n2)C3)CC2(CCC2)C1] Starting materials: Int-123 and Int-76 Reaction conditions: D″ | LCMS: 517.15 [M + H]⁺; ¹HNMR (CD3OD, 400 MHz): δ 8.53 (s, 1H), 8.15 (s, 1H), 7.56 (d, 1H), 7.47 (d, 1H), 7.26-7.24 (m, 2H), 5.09 (s, 2H), 3.94 (s, 2H), 3.25 (s, 2H), 2.85 (d, 2H), 2.73 (s, 3H), 2.56 (d, 2H), 2.50 (d, 2H), 2.40 - 2.30 (m, 2H), 1.94-1.91 (m, 4H), 1.05-1.00 (m, 1H), 0.59-0.53 (m, 2H), 0.24-0.20 (m, 2H). |
| 256 | 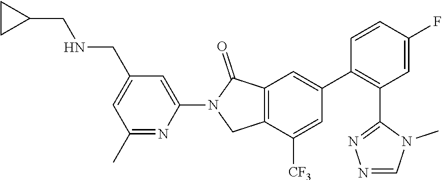 [SMILES: Cc1cc(CNCC2CC2)cc(N2Cc3c(cc(-c4ccc(F)cc4-c4nncn4C)cc3C(F)(F)F)C2=O)n1] Starting materials: Int-151 and Int-142 Reaction conditions: C″ | LCMS: 551.20 [M + H]⁺; ¹H-NMR (400 MHz, DMSO-d6): δ 8.50 (s, 1H), 8.32 (s, 1H), 7.86-7.81 (m, 2H), 7.66- 7.59 (m, 3H), 7.07 (s, 1H), 5.23 (s, 2H), 3.76 (s, 2H), 3.35 (s, 3H), 2.49 (s, 3H), 2.38 (d, 2H), 0.91-0.84 (m, 1H), 0.4-0.39 (m, 2H), 0.13-0.10 (m, 2H). |
| 257 | 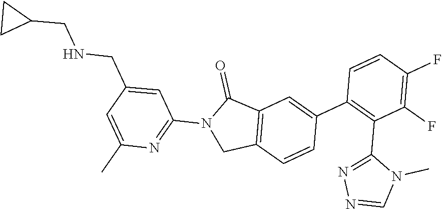 [SMILES: Cc1cc(CNCC2CC2)cc(N2Cc3ccc(-c4ccc(F)c(F)c4-c4nncn4C)cc3C2=O)n1] Starting materials: Int-151 and Int-139 Reaction conditions: D″ | LCMS: 501.20 [M + H]⁺; ¹H-NMR (400 MHz, DMSO-d6): δ 8.56 (s, 1H), 8.31 (s, 1H), 7.89-7.81 (m, 1H), 7.64 (d, 1H), 7.58-7.55 (m, 1H), 7.52 (s, 1H), 7.39 (dd, 1H), 7.04 (s, 1H), 5.08 (s, 2H), 3.76 (s, 2H), 3.32 (s, 3H), 2.45 (s, 3H), 2.39 (d, 2H), 0.95-0.90 (m, 1H), 0.4-0.40 (m, 2H), 0.15-0.10 (m, 2H). |
| 258 | 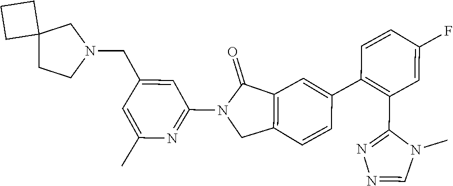 [SMILES: Cc1cc(CN2CCC3(CCC3)C2)cc(N2Cc3ccc(-c4ccc(F)cc4-c4nncn4C)cc3C2=O)n1] Starting materials: Int-236 and Int-236 Reaction conditions: C″ | LCMS: 523.2 [M + H]⁺; ¹H-NMR (400 MHz, DMSO-d6): δ 8.44 (s, 1H), 8.31 (s, 1H), 7.75-7.71 (m, 1H), 7.64 (d, 1H), 7.61-7.58 (m, 1H), 7.53-7.50 (m, 2H), 7.37 (dd, 1H), 6.99 (s, 1H), 5.08 (s, 2H), 3.58 (s, 2H), 3.14 (s, 3H), 2.52-2.50 (m, 4H), 2.46 (s, 3H), 1.98 - 1.89 (m, 4H), 1.86-1.76 (m, 4H). |
| 259 | 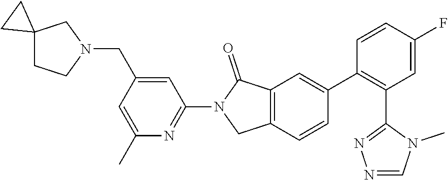 [SMILES: Cc1cc(CN2CCC3(CC3)C2)cc(N2Cc3ccc(-c4ccc(F)cc4-c4nncn4C)cc3C2=O)n1] Starting materials: Int-235 and Int-236 Reaction conditions: C″ | LCMS: 509.2 [M + H]⁺; ¹H-NMR (400 MHz, DMSO-d6): δ 8.44 (s, 1H), 8.32 (s, 1H), 7.75-7.71 (m, 1H), 7.64 (d, 1H), 7.61-7.50 (m, 3H), 7.38 (dd, 1H), 7.02 (s, 1H), 5.08 (s, 2H), 3.61 (s, 2H), 3.14 (s, 3H), 2.71-2.68 (m, 2H), 2.46 (s, 5H), 1.79-1.75 (m, 2H), 0.55- 0.49 (m, 4H). |
| 260 | 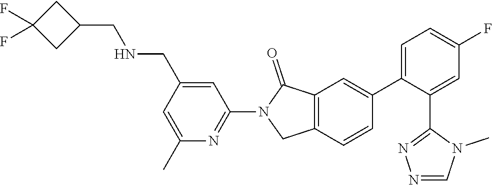 [SMILES: Cc1cc(CNCC2CC(F)(F)C2)cc(N2Cc3ccc(-c4ccc(F)cc4-c4nncn4C)cc3C2=O)n1] Starting materials: Int-237 and Int-99 Reaction conditions: C″ | LCMS: 533.3 [M + H]⁺; ¹H-NMR (400 MHz, DMSO-d6): δ 8.44 (s, 1H), 8.32 (s, 1H), 7.73 (dd, 1H), 7.64 (d, 1H), 7.65-7.52 (m, 1H), 7.52-7.50 (m, 2H), 7.38 (dd, 1H), 7.03 (s, 1H), 5.08 (s, 2H), 3.74 (s, 2H), 3.15 (s, 3H), 2.68 - 2.56 (m, 4H), 2.46 (s, 3H), 1.38- 1.20 (m, 3H). |
| 261 | 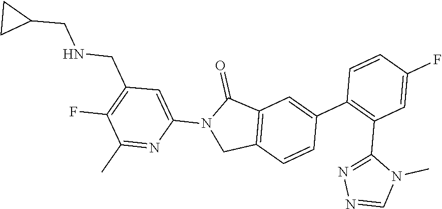 [SMILES: Cc1nc(N2Cc3ccc(-c4ccc(F)cc4-c4nncn4C)cc3C2=O)cc(CNCC2CC2)c1F] Starting materials: Int-160 and Int-99 Reaction conditions: C″ | LCMS: 501.15 [M + H]⁺; ¹H-NMR (400 MHz, DMSO-d6): δ 8.50 (d, 1H), 8.44 (s, 1H), 7.75-7.71 (m, 1H), 7.64 (d, 1H), 7.60-7.50 (m, 3H), 7.39 (dd,1H), 5.07 (s, 2H), 3.82 (s, 2H), 3.14 (s, 3H) 2.45-2.43 (m, 5H), 0.96 - 0.91 (m, 1H), 0.45-0.40 (m, 2H), 0.15-0.10 (m, 2H). |
| 262 | 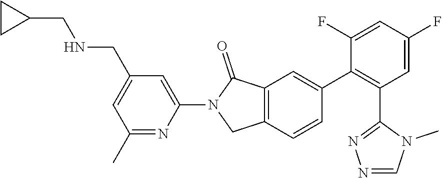 [SMILES: Cc1cc(CNCC2CC2)cc(N2Cc3ccc(-c4c(F)cc(F)cc4-c4nncn4C)cc3C2=O)n1] Starting materials: Int-151 and Int-140 Reaction conditions: B″ | LCMS: 501.20 [M + H]⁺; ¹H-NMR (400 MHz, DMSO-d6): δ 8.41 (s, 1H), 8.30 (bs, 1H), 7.73-7.65 (m, 2H), 7.57 (s, 1H), 7.48-7.40 (m, 2H), 7.04 (s, 1H), 5.08 (s, 2H), 3.75 (s, 2H), 3.28 (s, 3H) 2.45 (s, 3H), 2.39 (d, 2H), 0.93 - 0.89 (m, 1H), 0.43-0.39 (m, 2H), 0.14-0.10 (m, 2H). |
| 263 | 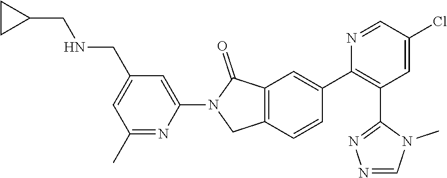 [SMILES: Cc1cc(CNCC2CC2)cc(N2Cc3ccc(-c4ncc(Cl)cc4-c4nncn4C)cc3C2=O)n1] Starting materials: Int-238 and Int-141 Reaction conditions: A″ | LCMS: 500.20 [M + H]⁺; ¹H-NMR (400 MHz, DMSO-d6): δ 9.00 (d, 1H), 8.56 (s, 1H), 8.32 (s, 1H), 8.29 (d, 1H), 7.70-7.66 (m, 2H), 7.55 (dd, 1H), 7.05 (s, 1H), 5.11 (s, 2H), 3.76 (s, 2H), 3.19 (s, 3H), 2.46 (s, 3H), 2.40 (d, 2H), 0.94-0.90 (m, 1H), 0.44-0.39 (m, 2H), 0.14-0.10 (m, 2H). |
| 264 | 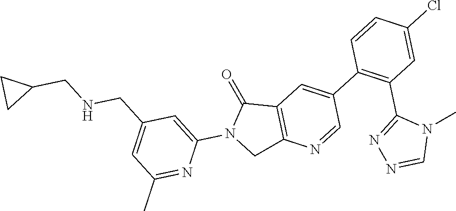 [SMILES: Cc1cc(CNCC2CC2)cc(N2Cc3ncc(-c4ccc(Cl)cc4-c4nncn4C)cc3C2=O)n1] Starting materials: Int-238 and Int-143 Reaction conditions: D″ | LCMS: 500.2 [M + H]⁺; ¹H-NMR (400 MHz, CDCl₃): δ 8.58 (d, 1H), 8.30 (s, 1H), 8.09 (s, 1H), 8.03 (d, 1H) 7.71 (d, 1H), 7.69 (dd, 1H), 7.56 (d, 1H), 7.04 (s, 1H), 5.16 (s, 2H), 3.89 (s, 2H), 3.24 (s, 3H), 2.57 (d, 2H), 2.56 (s, 3H), 1.06 - 1.02 (m, 1H), 0.56-0.51 (m, 2H), 0.19-0.17 (m, 2H). |
| 265 | 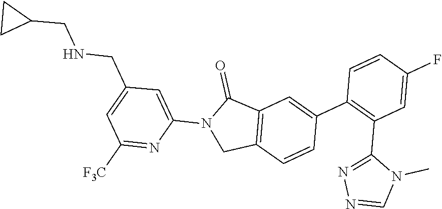 [SMILES: Cn1cnnc1-c1cc(F)ccc1-c1ccc2c(c1)C(=O)N(c1cc(CNCC3CC3)cc(C(F)(F)F)n1)C2] Starting materials: Int-239 and Int-99 Reaction conditions: A″ | LCMS: 537.2 [M + H]⁺; ¹H-NMR (400 MHz, DMSO-d6): δ 8.78 (s, 1H), 8.45 (s, 1H), 7.74 (dd, 1H), 7.70-7.53 (m, 4H), 7.51 (d, 1H),7.43 (dd, 1H), 5.10 (s, 2H), 3.90 (s, 2H), 3.16 (s, 3H), 2.41 (d, 2H), 0.94-0.86 (m, 1H), 0.42- 0.40 (m, 2H), 0.14-0.10 (m, 2H). |
| 266 | 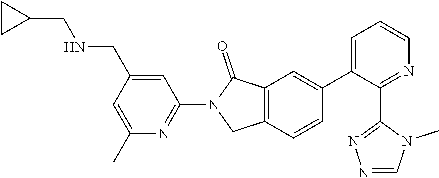 [SMILES: Cc1cc(CNCC2CC2)cc(N2Cc3ccc(-c4cccnc4-c4nncn4C)cc3C2=O)n1] Starting materials: Int-238 and Int-131 Reaction conditions: C″ | LCMS: 466.4 [M + H]⁺; ¹H-NMR (400 MHz, DMSO-d6): δ 8.81 (dd, 1H), 8.53 (s, 1H), 8.33 (s, 1H), 8.10 (dd, 1H), 7.72-7.69 (m, 1H), 7.67-7.63 (m, 2H), 7.50 (dd, 1H), 7.05 (s, 1H), 5.10 (s, 2H), 3.77 (s, 2H), 3.66 (s, 3H), 2.46 (s, 3H), 2.41 (d, 2H), 0.94-0.90 (m, 1H), 0.44-0.39 (m, 2H), 0.14- 0.10 (m, 2H). |
| 267 | 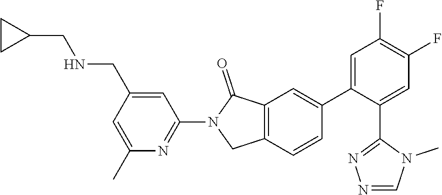 [SMILES: Cc1cc(CNCC2CC2)cc(N2Cc3ccc(-c4cc(F)c(F)cc4-c4nncn4C)cc3C2=O)n1] Starting materials: Int-238 and Int-132 Reaction conditions: D″ | LCMS: 501.20 [M + H]⁺; ¹H-NMR (400 MHz, DMSO-d6): δ 8.45 (s, 1H), 8.31 (s, 1H), 7.89-7.75 (m, 2H), 7.65 (d, 1H), 7.54 (d, 1H), 7.40 (dd, 1H), 7.04 (s,1H), 5.09 (s, 2H), 3.76 (s, 2H), 3.15 (s, 3H), 2.45 (s, 3H), 2.40 (d, 2H), 0.93-0.85 (m, 1H), 0.45-0.35 (m, 2H), 0.16-0.10 (m, 2H). |
| 268 | 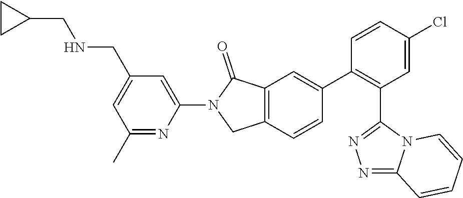 [SMILES: Cc1cc(CNCC2CC2)cc(N2Cc3ccc(-c4ccc(Cl)cc4-c4nnc5ccccn45)cc3C2=O)n1] Starting materials: Int-238 and Int-248 Reaction conditions: A″ | LCMS: 535.20 [M + H]⁺; ¹H-NMR (400 MHz, DMSO-d6): δ 8.26 (s, 1H), 7.89 - 7.84 (m, 3H), 7.77-7.72 (m, 2H), 7.52 - 7.49 (m, 2H), 7.32-7.25 (m, 2H), 7.02 (s, 1H), 6.77-6.74 (m, 1H), 4.98 (s, 2H), 3.74 (s, 2H), 2.44 (s, 3H), 2.43- 2.38 (d, 2H), 0.91-0.86 (m, 1H), 0.43 - 0.39 (m, 2H), 0.13-0.09 (m, 2H). |
| 269 | 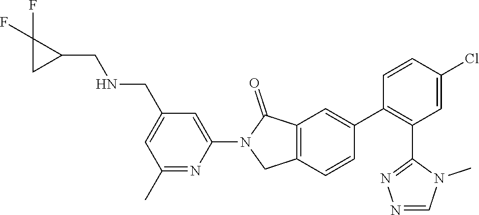 [SMILES: Cc1cc(CNCC2CC2(F)F)cc(N2Cc3ccc(-c4ccc(Cl)cc4-c4nncn4C)cc3C2=O)n1] Starting materials: Int-240 and Int-93 Reaction conditions: A″ | LCMS: 535.3 [M + H]⁺; ¹H-NMR (400 MHz, DMSO-d6): δ 8.44 (s, 1H), 8.31 (s, 1H), 7.80-7.78 (m, 1H), 7.72- 7.62 (m, 3H), 7.66-7.64 (m, 1H), 7.40 (dd, 1H), 7.05 (s, 1H), 5.08 (s, 2H), 3.76-3.74 (m, 2H), 3.12 (s, 3H), 2.70- 2.52 (m, 2H), 2.49 (s, 3H), 1.88-1.82 (m, 1H), 1.55-1.52 (m, 1H), 1.25- 1.18 (m, 1H). |
| 270 | 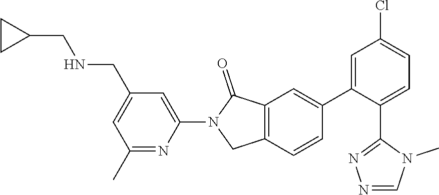 [SMILES: Cc1cc(CNCC2CC2)cc(N2Cc3ccc(-c4cc(Cl)ccc4-c4nncn4C)cc3C2=O)n1] Starting materials: Int-238 and Int-133 Reaction conditions: D″ | LCMS: 499.2 [M + H]⁺; ¹H-NMR (400 MHz, DMSO-d₆): δ 8.43 (s, 1H), 8.31 (s, 1H), 7.78 (d, 1H), 7.70-7.62 (m, 3H), 7.55 (s, 1H), 7.44 (dd, 1H), 7.04 (s, 1H), 5.10 (s, 2H), 3.76 (s, 2H), 3.09 (s, 3H), 2.45 (s, 3H), 2.40 (d, 2H), 0.94 - 0.86 (m, 1H), 0.44-0.39 (m, 2H), 0.13-0.10 (m, 2H). |
| 271 | 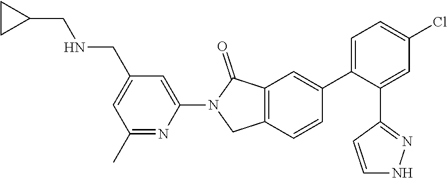 [SMILES: Cc1cc(CNCC2CC2)cc(N2Cc3ccc(-c4ccc(Cl)cc4-c4cc[nH]n4)cc3C2=O)n1] Starting materials: Int-238 and Int-146 Reaction conditions: A″ | LCMS: 484.2 [M + H]⁺; ¹H-NMR (400 MHz, DMSO-d₆): δ 12.9 (bs, 1H), 8.32 (s, 1H), 7.77 (bs, 1H), 7.67 (dd, 1H), 7.59 (bs, 1H), 7.55-7.40 (m, 4H), 7.05 (s, 1H), 5.68 (bs, 1H), 5.12 (s, 2H), 3.76 (s, 2H), 2.49 (s, 3H), 2.39 (d, 2H), 0.93-0.86 (m, 1H), 0.43-0.40 (m, 2H), 0.15-0.09 (m, 2H). |
| 272 | 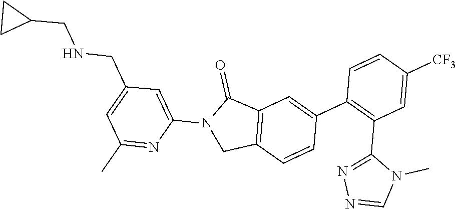 [SMILES: Cc1cc(CNCC2CC2)cc(N2Cc3ccc(-c4ccc(C(F)(F)F)cc4-c4nncn4C)cc3C2=O)n1] Starting materials: Int-238 and Int-134 Reaction conditions: D″ | LCMS: 533.3 [M + H]⁺; ¹H-NMR (400 MHz, DMSO-d6): δ 8.46 (s, 1H), 8.31 (s, 1H), 8.10 (d, 1H), 7.99 (s, 1H), 7.93 (d, 1H), 7.69 (d, 1H), 7.59 (s, 1H), 7.45 (d, 1H), 7.05 (s, 1H), 5.10 (s, 2H), 3.76 (s, 2H), 3.11 (s, 3H), 2.44 (s, 3H), 2.40 (d, 2H), 0.92-0.86 (m, 1H), 0.43- 0.39 (m, 2H), 0.1-0.10 (m, 2H). |
| 273 | 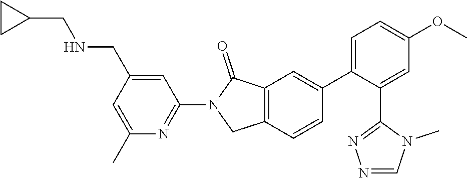 [SMILES: COc1ccc(-c2ccc3c(c2)C(=O)N(c2cc(CNCC4CC4)cc(C)n2)C3)c(-c2nncn2C)c1] Starting materials: Int-238 and Int-135 Reaction conditions: C″ | LCMS: 495.3 [M + H]⁺; ¹H-NMR (400 MHz, DMSO-d6): δ 8.42 (s, 1H), 8.04 (s, 1H), 7.64-7.58 (m, 2H), 7.45 (d, 1H), 7.35 (dd, 1H), 7.29 (dd, 1H), 7.14 (d,1H), 7.04 (d, 1H), 5.07 (s, 2H), 3.88 (s, 3H), 3.75 (s, 2H), 3.09 (s, 3H), 2.45 (s, 3H), 2.40 (d, 2H), 0.99-0.82 (m, 1H), 0.45-0.36 (m, 2H), 0.15-0.07 (m, 2H). |
| 274 | 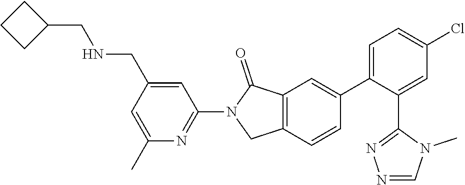 [SMILES: Cc1cc(CNCC2CCC2)cc(N2Cc3ccc(-c4ccc(Cl)cc4-c4nncn4C)cc3C2=O)n1] Starting materials: Int-241 and Int-93 Reaction conditions: C″ | LCMS: 513.4 [M + H]⁺; ¹H-NMR (400 MHz, DMSO-d6): δ 8.44 (s, 1H), 8.31 (s, 1H), 7.82-7.78 (m, 1H), 7.75- 7.69 (m, 2H), 7.68-7.62 (m, 1H), 7.53 (d, 1H), 7.40 (dd, 1H), 7.03 (s, 1H), 5.09 (s, 2H), 3.71 (s, 2H), 3.12 (s, 3H), 2.53 (d, 2H), 2.45 (s, 3H), 2.45-2.38 (m, 1H), 2.30-1.96 (m, 2H), 1.88- 1.75 (m, 2H), 1.70-1.58 (m, 2H). |
| 275 | 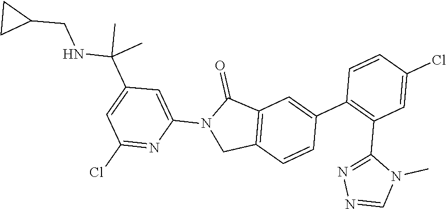 [SMILES: Cn1cnnc1-c1cc(Cl)ccc1-c1ccc2c(c1)C(=O)N(c1cc(C(C)(C)NCC3CC3)cc(Cl)n1)C2] Starting materials: Int-164 and Int-93 Reaction conditions: A″ | LCMS: 547.2 [M + H]⁺; ¹H-NMR (400 MHz, DMSO-d6): 8 8.59 (s, 1H), 8.44 (s, 1H), 7.80 (dd, 1H), 7.72-7.70 (m, 2H), 7.67 (d, 1H), 7.52 (s, 1H), 7.44 (dd, 1H), 7.38 (s, 1H), 5.06 (s, 2H), 3.12 (s, 3H), 2.22 (t, 1H), 2.10 (t, 2H), 1.37 (s, 6H), 0.88-0.84 (m, 1H), 0.40 - 0.35 (m, 2H), 0.07-0.05 (m, 2H). |
| 276 | 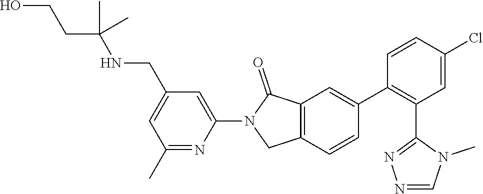 [SMILES: Cc1cc(CNC(C)(C)CCO)cc(N2Cc3ccc(-c4ccc(Cl)cc4-c4nncn4C)cc3C2=O)n1] Starting materials: Int-242 and Int-93 Reaction conditions: A″ | LCMS: 531.20 [M + H]⁺; ¹H-NMR (400 MHz, DMSO-d6): 8 8.44 (s, 1H), 8.33 (s, 1H), 7.80 (dd, 1H), 7.72-7.70 (m, 2H), 7.65 (d, 1H), 7.52 (d, 1H), 7.40 (dd, 1H), 7.05 (s, 1H), 5.08 (s, 2H), 3.68 (s, 2H), 3.56 (t, 2H), 3.12 (s, 3H), 2.45 (s, 3H), 1.60 (t, 2H), 1.09 (s, 6H). |
| 277 | 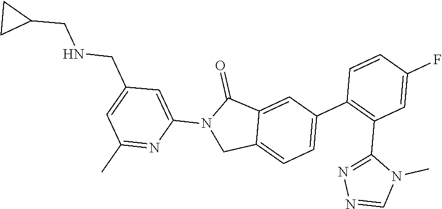 Starting materials: Int-238 and Int-136 Reaction conditions: D″ | LCMS: 479.30 [M + H]⁺; ¹H-NMR (400 MHz, CD₃OD): δ 8.37 (s, 1H), 8.32 (s, 1H), 7.71 (s, 1H), 7.60-7.71 (m, 3H), 7.47 (s, 1H), 7.45 (dd, 1H), 7.08 (s, 1H), 5.14 (s, 2H), 3.89 (s, 2H), 3.16 (s, 3H), 2.56 (d, 2H), 2.53 (s, 3H), 2.52 (s, 3H) 1.10-0.98 (m, 1H), 0.59-0.54 (m, 2H), 0.24-0.20 (m, 2H). |
| 278 | 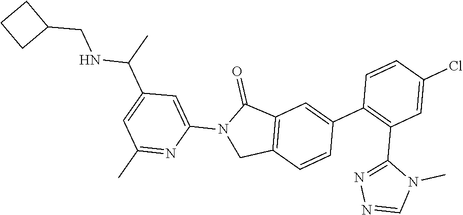 [SMILES: Cc1cc(C(C)NCC2CCC2)cc(N2Cc3ccc(-c4ccc(Cl)cc4-c4nncn4C)cc3C2=O)n1] Starting materials: Int-243 and Int-193 Reaction conditions: D″ | LCMS: 527.3 [M + H]⁺; ¹H-NMR (400 MHz, DMSO-d₆): δ 8.44 (s, 1H), 8.31 (s, 1H), 7.80 (dd, 1H), 7.78-7.70 (m, 2H), 7.65 (d, 1H), 7.51 (d, 1H), 7.40 (dd, 1H), 7.04 (s, 1H), 5.09 (s, 2H), 3.67 (q, 1H), 3.11 (s, 3H), 2.45 (s, 3H), 2.43-2.38 (m, 1H), 2.40-2.33 (m, 2H), 2.00-1.93 (m, 3H), 1.83-1.74 (m, 2H), 1.63-1.59 (m, 2H), 1.24 (d, 3H). |
| 279 | 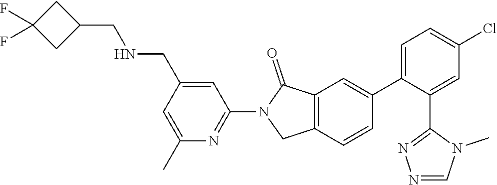 [SMILES: Cc1cc(CNCC2CC(F)(F)C2)cc(N2Cc3ccc(-c4ccc(Cl)cc4-c4nncn4C)cc3C2=O)n1] Starting materials: Int-244 and Int-93 Reaction conditions: A″ | LCMS: 549.2 [M + H]⁺; ¹H-NMR (400 MHz, DMSO-d6): δ 8.44 (s, 1H), 8.32 (s, 1H), 7.80 (dd, 1H), 7.72-7.70 (m, 2H), 7.65 (d, 1H), 7.53 (s, 1H), 7.39 (dd, 1H), 7.04 (s, 1H), 5.09 (s, 2H), 3.73 (s, 2H), 3.12 (s, 3H), 2.65-2.58 (m, 4H), 2.45 (s, 3H), 2.30-2.22 (m, 2H). |
| 280 | 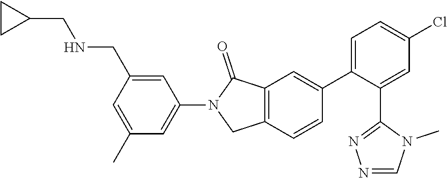 [SMILES: Cc1cc(CNCC2CC2)cc(N2Cc3ccc(-c4ccc(Cl)cc4-c4nncn4C)cc3C2=O)c1] Starting materials: Int-5 and Int-93 Reaction conditions: C″ | LCMS: 498.2 [M + H]⁺; ¹H-NMR (400 MHz, DMSO-d6): δ 8.44 (s, 1H), 7.80 (dd, 1H), 7.72-7.70 (m, 2H), 7.65 (s, 1H), 7.61-7.59 (m, 2H), 7.51 (s, 1H), 7.36 (dd, 1H), 6.99 (s, 1H), 5.01 (s, 2H), 3.72 (s, 2H), 3.11 (s, 3H), 2.40 (d, 2H), 2.34 (s, 3H), 0.94-0.86 (m, 1H), 0.42-0.40 (m, 2H), 0.12-0.10 (m, 2H). |
| 281 | 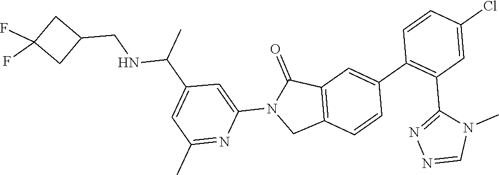 [SMILES: Cc1cc(C(C)NCC2CC(F)(F)C2)cc(N2Cc3ccc(-c4ccc(Cl)cc4-c4nncn4C)cc3C2=O)n1] Starting materials: Int-246 and Int-93 Reaction conditions: C″ | LCMS: 563.23 [M + H]⁺; ¹H-NMR (400 MHz, DMSO-d₆): δ 8.44 (s, 1H), 8.32 (s, 1H), 7.80 (dd, 1H), 7.72-7.67 (m, 2H), 7.65 (d, 1H), 7.52 (d, 1H), 7.40 (dd, 1H), 7.05 (s, 1H), 5.08 (s, 2H), 3.70 (q, 1H), 3.11 (s, 3H), 2.62- 2.57 (m, 2H), 2.46 (s, 3H), 2.40-2.19 (m, 5H), 1.25 (d, 3H). |
| 282 | 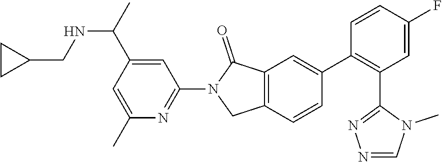 [SMILES: Cc1cc(C(C)NCC2CC2)cc(N2Cc3ccc(-c4ccc(F)cc4-c4nncn4C)cc3C2=O)n1] Starting materials: Int-226 and Int-99 Reaction conditions: C″ | LCMS: 497.20 [M + H]⁺; ¹H-NMR (400 MHz, DMSO-d6): δ 8.44 (s, 1H), 8.30 (s, 1H), 7.72 (dd, 1H), 7.64 (d, 1H), 7.59 (dt, 1H), 7.55-7.49 (m, 2H), 7.39 (dd, 1H), 7.04 (s, 1H), 5.08 (s, 2H), 3.75 (q, 1H), 3.14 (s, 3H), 2.45 (s, 3H), 2.30-2.18 (m, 2H), 2.15- 2.09 ( m, 1H), 1.26 (d, 3H), 0.90-0.82 (m, 1H), 0.42-0.32 (m, 2H), 0.09- 0.02 (m, 2H). |
| 283 | 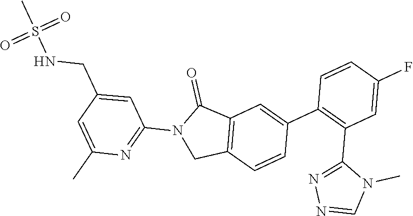 [SMILES: Cc1cc(CNS(C)(=O)=O)cc(N2Cc3ccc(-c4ccc(F)cc4-c4nncn4C)cc3C2=O)n1] Starting materials: Int-168 and Int-99 Reaction conditions: C″ | LCMS: 507.2 [M + H]⁺; ¹H-NMR (400 MHz, DMSO-d6): δ 8.45 (s, 1H), 8.35 (s, 1H), 7.78-7.72 (m, 2H), 7.65 (d, 1H), 7.58 (dt, 1H), 7.54-7.51 (m, 2H), 7.39 (dd, 1H), 7.04 (s, 1H), 5.09 (s, 2H), 4.22 (d, 2H), 3.15 (s, 3H), 2.96 (s, 3H) 2.48 (s, 3H). |
| 284 | 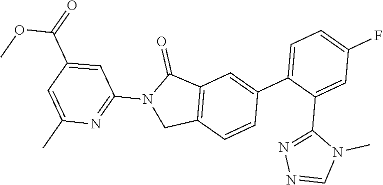 [SMILES: COC(=O)c1cc(C)nc(N2Cc3ccc(-c4ccc(F)cc4-c4nncn4C)cc3C2=O)c1] Starting materials: Int-151a and Int-99 Reaction conditions: C″ | LCMS: 458.1 [M + H]⁺; ¹H-NMR (400 MHz, DMSO-d6): δ 8.84 (s, 1H), 8.45 (s, 1H), 7.73 (dd, 1H) , 7.66 (d, 1H), 7.59 (dt, 1H), 7.55-7.51 (m, 3H), 7.41 (dd, 1H), 5.11 (s, 2H), 3.93 (s, 3H), 3.16 (s, 3H), 2.57 (s, 3H). |
| 285 | 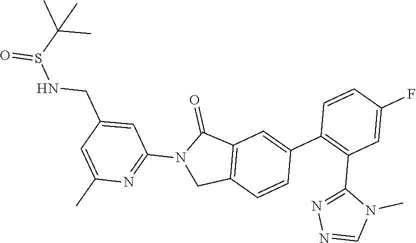 [SMILES: Cc1cc(CNS(=O)C(C)(C)C)cc(N2Cc3ccc(-c4ccc(F)cc4-c4nncn4C)cc3C2=O)n1] Starting materials: Int-167 and Int-99 Reaction conditions: C″ | LCMS: 533.30 [M + H]⁺; ¹H-NMR (400 MHz, DMSO-d6): δ 8.44 (s, 1H), 8.38 (s, 1H), 7.74 (dd, 1H), 7.64 (d, 1H), 7.61-7.50 (m, 3H), 7.37 (dd, 1H), 7.04 (s, 1H), 5.93 (t, 1H), 5.08 (s, 2H), 4.27-4.15 (m, 2H), 3.14 (s, 3H), 2.46 (s, 3H), 1.19 (s, 9H). |
| 286 | 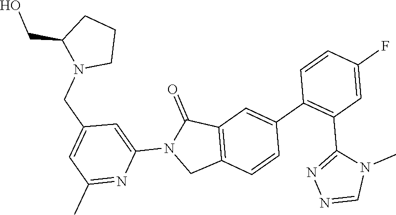 [SMILES: Cc1cc(CN2CCC[C@@H]2CO)cc(N2Cc3ccc(-c4ccc(F)cc4-c4nncn4C)cc3C2=O)n1] Starting materials: Int-227 and Int-99 Reaction conditions: C″ | LCMS: 513.3 [M + H]⁺; ¹H-NMR (400 MHz, DMSO-d6): δ 8.44 (s, 1H), 8.30 (s, 1H), 7.73 (dd, 1H), 7.65 (d, 1H), 7.59 (dt, 1H), 7.52 (dd, 1H), 7.49 (s, 1H), 7.40 (s, 1H), 7.03 (s, 1H), 5.08 (s, 2H), 4.47 (t, 1H), 4.11 (d, 1H), 3.52- 3.46 (m, 1H), 3.34 (d, 1H), 3.14 (s, 3H), 2.90-2.85 (m, 1H), 2.65-2.55 (m, 1H), 2.45 (s, 3H), 2.22-2.15 (m, 1H), 1.91-1.82 (m, 1H), 1.70-1.52 (m, 3H). |
| 287 | 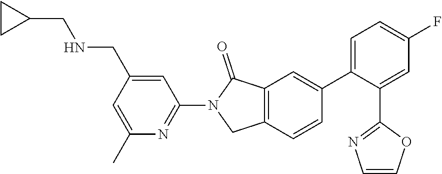 [SMILES: Cc1cc(CNCC2CC2)cc(N2Cc3ccc(-c4ccc(F)cc4-c4ncco4)cc3C2=O)n1] Starting materials: Int-151 and Int-147 Reaction conditions: C″ | LCMS: 469.2 [M + H]⁺; ¹H-NMR (400 MHz, DMSO-d6): δ 8.34 (s, 1H), 8.05 (s, 1H), 7.75 (dd, 1H), 7.71 (d, 1H), 7.60-7.48 (m, 4H), 7.31 (s, 1H), 7.06 (s,1H), 5.14 (s, 2H), 3.79 (s, 2H), 2.47 (s, 3H), 2.43 (d, 2H), 0.98-0.88 (m, 1H), 0.45 0.40 (m, 2H), 0.16-0.11 (m, 2H). |
| 288 | 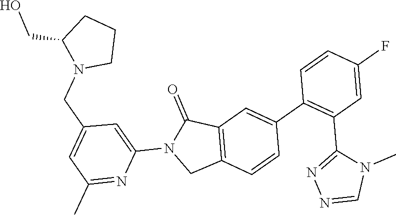 [SMILES: Cc1cc(CN2CCC[C@H]2CO)cc(N2Cc3ccc(-c4ccc(F)cc4-c4nncn4C)cc3C2=O)n1] Starting materials: Int-228 and Int-99 Reaction conditions: C″ | LCMS: 513.2 [M + H]⁺; ¹H-NMR (400 MHz, DMSO-d6): δ 8.44 (s, 1H), 8.29 (s, 1H), 7.73 (dd, 1H), 7.65 (d, 1H), 7.58 (dt, 1H), 7.50 (dd, 1H), 7.48 (s, 1H), 7.40 (s, 1H), 7.03 (s, 1H), 5.08 (s, 2H), 4.47 (t, 1H), 4.11 (d, 1H), 3.52- 3.45 (m, 1H), 3.38 (d, 1H), 3.14 (s, 3H), 2.90-2.84 (m, 1H), 2.63-2.58 (m, 1H), 2.46 (s, 3H), 2.22-2.12 (m, 1H), 1.91-1.81 (m, 1H), 1.70-1.52 (m, 3H). |
| 289 | 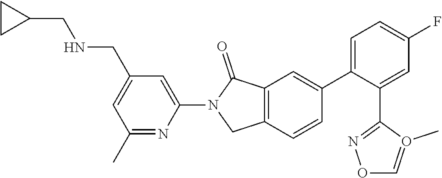 Starting materials: Int-151 and Int-149 Reaction conditions: C″ | LCMS: 483.2 [M + H]⁺; ¹H-NMR (400 MHz, DMSO-d6): δ 8.66 (s, 1H), 8.31 (s, 1H), 7.72-7.65 (m, 2H), 7.57-7.47 (m, 3H), 7.44 (dd, 1H), 7.04 (s, 1H), 5.10 (s, 2H), 3.75 (s, 2H), 2.42 (s, 3H), 2.39 (d, 2H), 1.46 (s, 3H), 0.95-0.85 (m, 1H), 0.42-0.38 (m, 2H), 0.14-0.09 (m, 2H). |
| 290 | 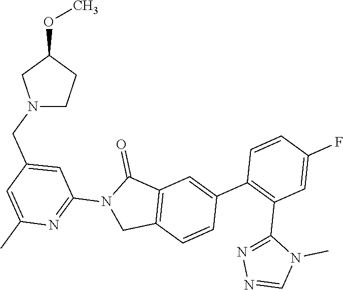 [SMILES: CO[C@H]1CCN(Cc2cc(C)nc(N3Cc4ccc(-c5ccc(F)cc5-c5nncn5C)cc4C3=O)c2)C1] Starting materials: Int-229 and Int-99 Reaction conditions: C″ | LCMS: 513.20 [M + H]⁺; ¹H-NMR (400 MHz, DMSO-d₆): δ 8.45 (s, 1H), 8.29 (s, 1H), 7.74 (dd, 1H), 7.64 (d, 1H), 7.59 (dt, 1H), 7.55-7.48 (m, 2H), 7.39 (dd, 1H), 7.00 (s, 1H), 5.08 (s, 2H), 3.95-3.89 (m, 1H), 3.60 (s, 2H), 3.17 (s, 3H), 3.14 (s, 3H), 2.78- 2.72 (m, 1H), 2.62-2.54 (m, 1H), 2.49 - 2.42 (s, 5H), 2.05-1.97 (m, 1H), 1.73-1.65 (m, 1H). |
| 291 | 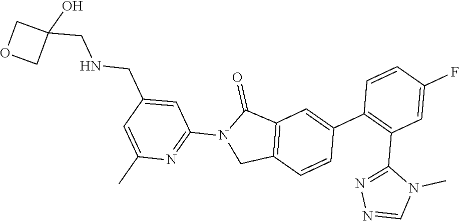 [SMILES: Cc1cc(CNCC2(O)COC2)cc(N2Cc3ccc(-c4ccc(F)cc4-c4nncn4C)cc3C2=O)n1] Starting materials: Int-230 and Int-99 Reaction conditions: C″ | LCMS: 515.20 [M + H]⁺; ¹H-NMR (400 MHz, DMSO-d6): δ 8.44 (s, 1H), 8.35 (s, 1H), 7.74 (dd, 1H), 7.62 (d, 1H), 7.56 (dt, 1H), 7.55-7.49 (m, 2H), 7.38 (dd, 1H), 7.05 (s, 1H), 5.68 (s, 1H), 5.09 (s, 2H), 4.42-4.38 (m, 4H), 3.81 (s, 2H), 3.14 (s, 3H), 2.75 (s, 2H), 2.46 (s, 3H). |
| 292 | 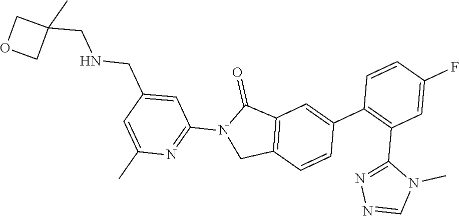 [SMILES: Cc1cc(CNCC2(C)COC2)cc(N2Cc3ccc(-c4ccc(F)cc4-c4nncn4C)cc3C2=O)n1] Starting materials: Int-231 and Int-99 Reaction conditions: C″ | LCMS: 513.2 [M + H]⁺; ¹H-NMR (400 MHz, DMSO-d6): δ 8.44 (s, 1H), 8.36 (s, 1H), 7.74 (dd, 1H), 7.64 (d, 1H), 7.58 (dt, 1H), 7.54-7.50 (m, 2H), 7.37 (dd, 1H), 7.05 (s, 1H), 5.09 (s, 2H), 4.37 (d, 2H), 4.19 (d, 2H), 3.77 (s, 2H), 3.14 (s, 3H), 2.64 (s, 2H), 2.46 (s, 3H), 1.27 (s, 3H). |
| 293 | 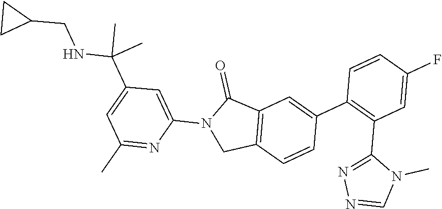 [SMILES: Cc1cc(C(C)(C)NCC2CC2)cc(N2Cc3ccc(-c4ccc(F)cc4-c4nncn4C)cc3C2=O)n1] Starting materials: Int-163 and Int-93 Reaction conditions: C″ | LCMS: 511.2 [M + H]⁺; ¹H-NMR (400 MHz, DMSO-d₆): δ 8.44 (s, 1H), 8.43 (s, 1H), 7.72 (dd, 1H), 7.65 (d, 1H), 7.59 (dt, 1H), 7.52 (dd, 1H), 7.49 (s, 1H), 7.40 (dd, 1H), 7.16 (s, 1H), 5.08 (s, 2H), 3.13 (s, 3H), 2.46 (s, 3H), 2.11 (d, 2H), 1.36 (s, 6H), 0.90-0.82 (m, 1H), 0.40-0.35 (m, 2H), 0.08-0.02 (m, 2H). |
| 294 | 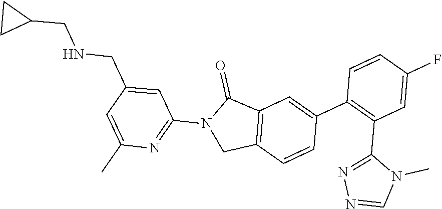 Starting materials: Int-151 and Int-137 Reaction conditions: C″ | LCMS: 497.2 [M + H]⁺; ¹H-NMR (400 MHz, DMSO-d₆): δ 8.55 (s, 1H), 8.32 (s, 1H), 7.73 (dd, 1H), 7.65 (d, 1H), 7.59 (dt, 1H), 7.55-7.48 (m, 2H), 7.45- 7.35 (m, 1H), 7.05 (s, 1H), 5.08 (s, 2H), 3.79 (s, 2H), 3.51-3.42 (m, 2H), 2.46 (s, 3H), 2.43 (d, 2H), 0.99-0.90 (m, 4H), 0.45-0.40 (m, 2H), 0.15-0.10 (m, 2H). |
| 295 | 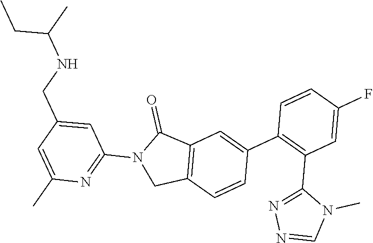 [SMILES: CCC(C)NCc1cc(C)nc(N2Cc3ccc(-c4ccc(F)cc4-c4nncn4C)cc3C2=O)c1] Starting materials: Int-232 and Int-99 Reaction conditions: C″ | LCMS: 485.3 [M + H]⁺; ¹H-NMR (400 MHz, CD3OD): δ 8.40 (s, 1H), 8.32 (d, 1H), 7.76 (dd, 1H), 7.71 (s, 1H), 7.62 (d, 1H), 7.54 (dt, 1H), 7.48-7.08 (m, 2H), 7.08 (s, 1H), 5.15 (s, 2H), 3.83 (ABq, 2H), 3.20 (s, 3H), 2.66-2.59 (m, 1H), 2.53 (s, 3H), 1.68-1.60 (m, 1H), 1.42-1.30 (m, 1H), 1.13 (d, 3H), 0.95 (t, 3H). |
| 296 | 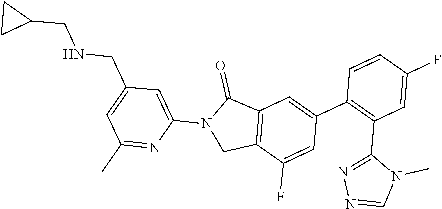 [SMILES: Cc1cc(CNCC2CC2)cc(N2Cc3c(F)cc(-c4ccc(F)cc4-c4nncn4C)cc3C2=O)n1] Starting materials: Int-151 and Int-144 Reaction conditions: C″ | LCMS: 501.20 [M + H]⁺; ¹H-NMR (400 MHz, DMSO-d6): δ 8.49 (s, 1H), 8.29 (s, 1H), 7.77 (dd, 1H), 7.62- 7.52 (m, 2H), 7.33 (s,1H), 7.29 (dd,1H), 7.07 (s,1H), 5.14 (s, 2H), 3.77 (s, 2H), 3.26 (s, 3H) 2.46 (s, 3H), 2.40 (d, 2H), 0.95-0.85 (m, 1H), 0.44 - 0.39 (m, 2H), 0.14-0.09 (m, 2H). |
| 297 | 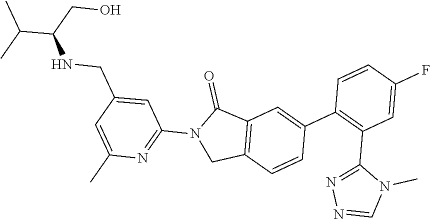 [SMILES: Cc1cc(CN[C@H](CO)C(C)C)cc(N2Cc3ccc(-c4ccc(F)cc4-c4nncn4C)cc3C2=O)n1] Starting materials: Int-233 and Int-99 Reaction conditions: C″ | LCMS: 515.1 [M + H]⁺; ¹H-NMR (400 MHz, DMSO-d6): δ 8.44 (s, 1H), 8.36 (brs, 1H), 7.74 (dd, 1H), 7.64 (d, 1H), 7.58 (dt, 1H), 7.55-7.50 (m, 2H), 7.39 (dd, 1H), 7.07 (s, 1H), 5.09 (s, 2H), 4.46-4.39 (m, 1H), 3.90-3.70 (m, 2H), 3.52-3.45 (m, 1H), 3.40-3.35 (m, 1H), 3.15 (s, 3H), 2.46 (s, 3H), 2.34-2.24 (m, 1H), 1.85-1.75 (m, 1H), 0.91 (d, 3H), 0.86 (d, 3H). |
| 298 | 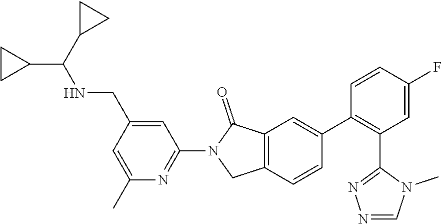 [SMILES: Cc1cc(CNC(C2CC2)C2CC2)cc(N2Cc3ccc(-c4ccc(F)cc4-c4nncn4C)cc3C2=O)n1] Starting materials: Compound 137a Reaction conditions: E″ | LCMS: 523.30 [M + H]⁺; ¹H-NMR (400 MHz, DMSO-d6): δ 8.45 (s, 1H), 8.32 (s, 1H), 7.75-7.72 (m, 1H), 7.63 (d, 1H), 7.61-7.58 (m, 1H), 7.57-7.50 (m, 2H), 7.38 (dd, 1H), 7.05 (s, 1H), 5.08 (s, 2H), 3.89 (s, 2H), 3.14 (s, 3H), 2.45 (s, 3H), 0.84-0.80 (m, 2H), 0.44-0.34 (m, 4H), 0.23-0.18 (m, 4H). |
| 299 | 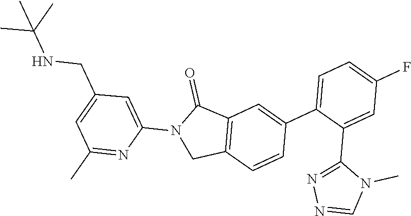 [SMILES: Cc1cc(CNC(C)(C)C)cc(N2Cc3ccc(-c4ccc(F)cc4-c4nncn4C)cc3C2=O)n1] Starting materials: Int-234 and Int-99 Reaction conditions: C″ | LCMS: 485.3 [M + H]⁺; ¹H-NMR (400 MHz, DMSO-d6): δ 8.45 (s, 1H), 8.34 (s, 1H), 7.79-7.70 (m, 1H), 7.64 (d, 1H), 7.62-7.55 (m, 1H), 7.54-7.48 (m, 2H), 7.39 (dd, 1H), 7.06 (s, 1H), 5.08 (s, 2H), 3.69 (s, 2H), 3.15 (s, 3H), 2.45 (s, 3H), 1.10 (s, 9H). |
| 300 | 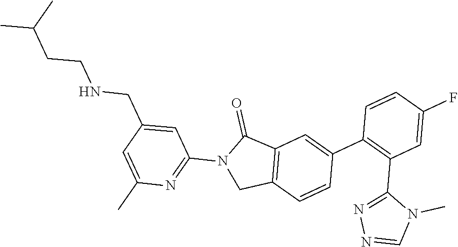 [SMILES: Cc1cc(CNCCC(C)C)cc(N2Cc3ccc(-c4ccc(F)cc4-c4nncn4C)cc3C2=O)n1] Starting materials: Compound 137a Reaction conditions: E″ | LCMS: 499.2 [M + H]⁺; ¹H-NMR (400 MHz, CD3OD): δ 8.40 (s, 1H), 8.31 (s, 1H), 7.76-7.75 (m, 1H), 7.64 (s, 1H), 7.62 (d, 1H), 7.58-7.50 (m, 1H), 7.48 - 7.44 (m, 2H), 7.07 (s, 1H), 5.16 (s, 2H), 3.83 (s, 2H), 3.20 (s, 3H), 2.66 (t, 2H), 2.53 (s, 3H), 1.65-1.63 (m, 1H), 1.51-1.30 (m, 2H), 0.94 (d, 6H). |
| 301 | 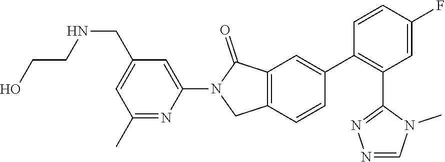 [SMILES: Cc1cc(CNCCO)cc(N2Cc3ccc(-c4ccc(F)cc4-c4nncn4C)cc3C2=O)n1] Starting materials: Compound 137a Reaction conditions: E″ | LCMS: 473.2 [M + H]⁺; ¹H-NMR-400 MHz, DMSO-d₆): δ 8.45 (s, 1H), 8.31 (s, 1H), 7.73 (dd, 1H), 7.64 (d, 1H), 7.61-7.58 (m, 1H), 7.56-7.50 (m, 2H), 7.39 (dd, 1H), 7.04 (s, 1H), 5.08 (s, 2H), 4.51 (t, 1H), 3.75 (s, 2H), 3.49 (q, 2H), 3.15 (s, 3H), 2.59 (t, 2H), 2.46 (s, 3H). |
| 302 | 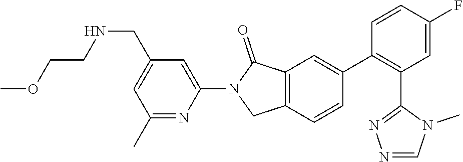 Starting materials: Compound 137a Reaction conditions: E″ | LCMS: 487.2 [M + H]⁺; ¹HNMR-400 MHz- DMSO-d₆): δ 8.44 (s, 1H), 8.30 (s, 1H), 7.73 (dd, 1H), 7.64 (d, 1H), 7.58 (td, 1H), 7.53-7.50 (m, 2H), 7.39 (dd, 1H), 7.04 (s, 1H), 5.08 (s, 2H), 3.75 (s, 2H), 3.42 (t, 2H), 3.26 (s, 3H), 3.14 (s, 3H), 2.67 (t, 2H), 2.46 (s, 3H). |
| 303 | 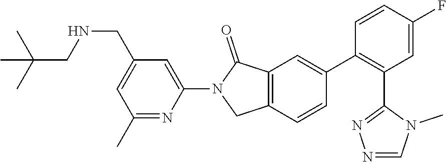 [SMILES: Cc1cc(CNCC(C)(C)C)cc(N2Cc3ccc(-c4ccc(F)cc4-c4nncn4C)cc3C2=O)n1] Starting materials: Compound 137a Reaction conditions: E″ | LCMS: 499.3 [M + H]⁺; ¹HNMR-400 MHz- DMSO-d₆): δ 8.44 (s, 1H), 8.33 (s, 1H), 7.75-7.71 (m, 1H), 7.64 (d, 1H), 7.60-7.56 (m, 1H), 7.53-7.50 (m, 2H), 7.37 (dd, 1H), 7.04 (s, 1H), 5.08 (s, 2H), 3.75 (s, 2H), 3.14 (s, 3H), 2.45 (s, 3H), 2.24 (s, 2H), 0.90 (s, 9H). |
| 304 | 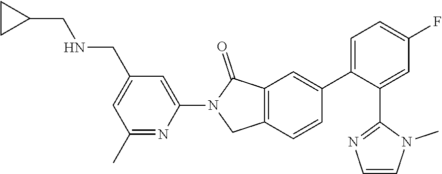 [SMILES: Cc1cc(CNCC2CC2)cc(N2Cc3ccc(-c4ccc(F)cc4-c4nccn4C)cc3C2=O)n1] Starting materials: Int-151 and Int-150 Reaction conditions: C″ | LCMS: 482.2 [M + H]⁺; ¹H-NMR (400 MHz, DMSO-d6): δ 8.31 (s, 1H), 7.68 (d, 1H), 7.62 (d, 1H), 7.50 (td, 1H), 7.45 (d, 1H), 7.41-7.34 (m, 2H), 7.07 (d, 1H), 7.04 (s, 1H), 6.95 (s, 1H), 5.08 (s, 2H), 3.76 (s, 2H), 3.03 (s, 3H), 2.45 (s, 3H), 2.39 (d, 2H), 0.95-0.86 (m, 1H), 0.43-0.39 (m, 2H), 0.14-0.09 (m, 2H). |
| 305 | 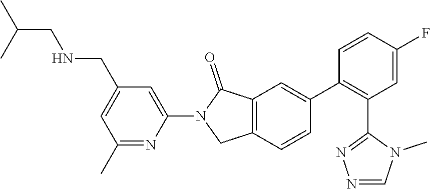 [SMILES: Cc1cc(CNCC(C)C)cc(N2Cc3ccc(-c4ccc(F)cc4-c4nncn4C)cc3C2=O)n1] Starting materials: Compound 137a Reaction conditions: E″ | LCMS: 485.2 [M + H]⁺; ¹H-NMR (400 MHz, DMSO-d6): δ 8.44 (s, 1H), 8.32 (s, 1H), 7.73-7.71 (m, 1H), 7.64 (d, 1H), 7.59-7.56 (m, 1H), 7.53-7.50 (m, 2H), 7.38 (dd, 1H), 7.04 (s, 1H), 5.08 (s, 2H), 3.72 (s, 2H), 3.15 (s, 3H), 2.45 (s, 3H), 2.31 (d, 2H), 1.75-1.65 (m, 1H), 0.89 (m, 6H). |
| 306 | 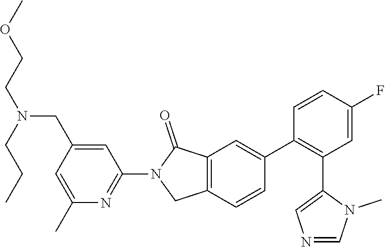 [SMILES: CCCN(CCOC)Cc1cc(C)nc(N2Cc3ccc(-c4ccc(F)cc4-c4cncn4C)cc3C2=O)c1] Starting materials: Compound 137a Reaction conditions: E″ | LCMS: 529.2 [M + H]⁺; ¹H-NMR (400 MHz, DMSO-d₆): δ 8.44 (s, 1H), 8.34 (s, 1H), 7.75-7.70 (m, 1H), 7.64 (d, 1H), 7.61-7.55 (m, 1H), 7.53-7.49 (m, 2H), 7.38 (dd, 1H), 7.01 (s, 1H), 5.08 (s, 2H), 3.63 (s, 2H), 3.45 (t, 2H), 3.23 (s, 3H), 3.14 (s, 3H), 2.62 (t, 2H), 2.45 (s, 3H), 2.42 (d, 2H), 1.50-1.40 (m, 2H), 0.85 (t, 3H). |
| 307 | 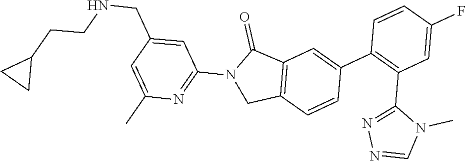 [SMILES: Cc1cc(CNCCC2CC2)cc(N2Cc3ccc(-c4ccc(F)cc4-c4nncn4C)cc3C2=O)n1] Starting materials: Compound 137a Reaction conditions: E″ | LCMS: 497.2 [M + H]⁺; ¹H-NMR (400 MHz, DMSO-d₆): δ 8.44 (s, 1H), 8.32 (s, 1H), 7.75-7.71 (m, 1H), 7.67 (d, 1H), 7.62-7.55 (m, 1H), 7.55-7.49 (m, 2H), 7.39 (dd, 1H), 7.05 (s, 1H), 5.09 (s, 2H), 3.74 (s, 2H), 3.15 (s, 3H), 2.59 (t, 2H), 2.46 (s, 3H), 1.40-1.30 (m, 2H), 0.78-0.68 (m, 1H), 0.41- 0.36 (m, 2H), 0.03-0.01 (m, 2H). |
| 308 | 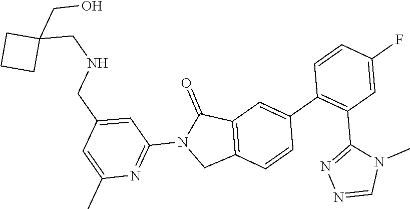 [SMILES: Cc1cc(CNCC2(CO)CCC2)cc(N2Cc3ccc(-c4ccc(F)cc4-c4nncn4C)cc3C2=O)n1] Starting materials: Compound 137a Reaction conditions: E″ | LCMS: 527.2 [M + H]⁺; ¹H-NMR (400 MHz, CD3OD): δ 8.40 (s, 1H), 8.32 (s, 1H), 7.77-7.73 (m, 1H), 7.71 (s,1H), 7.62 (d, 1H), 7.58-7.51 (m, 1H), 7.50 - 7.43 (m, 2H), 7.07 (s, 1H), 5.16 (s, 2H), 3.84 (s, 2H), 3.68 (s, 2H), 3.20 (s, 3H), 2.77 (s, 2H), 2.53 (s, 3H), 1.91- 1.83 (m, 6H). |
| 309 | 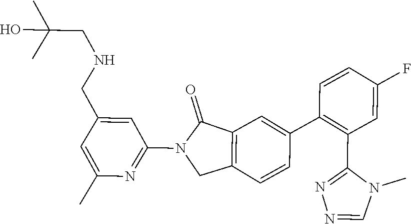 [SMILES: Cc1cc(CNCC(C)(C)O)cc(N2Cc3ccc(-c4ccc(F)cc4-c4nncn4C)cc3C2=O)n1] Starting materials: Compound 137a Reaction conditions: E″ | LCMS: 501.2 [M + H]⁺; ¹H-NMR (400 MHz, CD3OD): δ 8.40 (s, 1H), 8.33 (s, 1H), 7.77-7.75 (m, 1H), 7.71 (s, 1H), 7.63 (d, 1H), 7.56-7.54 (m, 1H), 7.48 - 7.44 (m, 2H), 7.09 (s, 1H), 5.16 (s, 2H), 3.87 (s, 2H), 3.20 (s, 3H), 2.58 (s, 2H), 2.53 (s, 3H), 1.25 (s, 6H). |
| 310 | 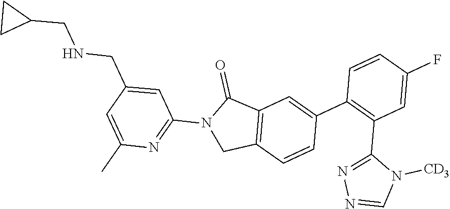 [SMILES: [2H]C([2H])([2H])n1cnnc1-c1cc(F)ccc1-c1ccc2c(c1)C(=O)N(c1cc(CNCC3CC3)cc(C)n1)C2] Starting materials: Int-151 and Int-148 Reaction conditions: C″ | LCMS: 486.2 [M + H]⁺; ¹H-NMR (400 MHz, DMSO-d₆): δ 8.44 (s, 1H), 8.31 (s, 1H), 7.73 (dd, 1H), 7.64 (d, 1H), 7.64-7.59 (m, 1H), 7.55-7.50 (m, 2H), 7.37 (dd, 1H), 7.04 (s, 1H), 5.08 (s, 2H), 3.75 (s, 2H), 2.45 (s, 3H), 2.39 (d, 2H), 0.98-0.82 (m, 1H), 0.43- 0.38 (m, 2H), 0.13-0.09 (m, 2H) |
| 311 | 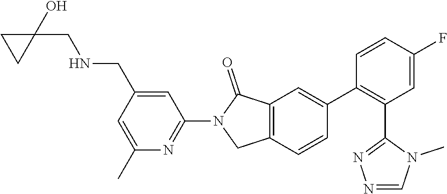 [SMILES: Cc1cc(CNCC2(O)CC2)cc(N2Cc3ccc(-c4ccc(F)cc4-c4nncn4C)cc3C2=O)n1] Starting materials: Compound 137a Reaction conditions: E″ | LCMS: 499.2 [M + H]⁺; 1H NMR (400 MHz, CD3OD): δ 8.40 (s, 1H), 8.34 (s, 1H), 7.75 (dd, 1H), 7.71 (s, 1H), 7.63 (d, 1H), 7.57-7.52 (m, 1H), 7.48- 7.44 (m, 2H), 7.10 (s, 1H), 5.16 (s, 2H), 3.95 (s, 2H), 3.20 (s, 3H), 2.78 (s, 2H), 2.53 (s, 3H), 0.79-0.74 (m, 2H), 0.62-0.57 (m, 2H). |
| 312 | 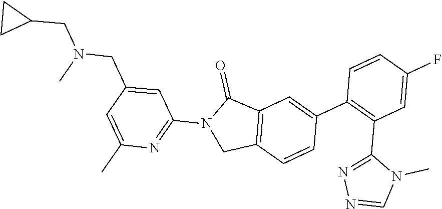 [SMILES: Cc1cc(CN(C)CC2CC2)cc(N2Cc3ccc(-c4ccc(F)cc4-c4nncn4C)cc3C2=O)n1] Starting materials: Compound 137a Reaction conditions: E″ | LCMS: 497.2 [M + H]⁺ ; ¹H-NMR (400 MHz, DMSO-d6): δ 8.44 (s, 1H), 8.33 (s, 1H), 7.75-7.71 (m, 1H), 7.64 (d, 1H), 7.61-7.56 (m, 1H), 7.53-7.50 (m, 2H), 7.39 (dd, 1H), 7.01 (s, 1H), 5.08 (s, 2H), 3.55 (s, 2H), 3.14 (s, 3H), 2.46 (s, 3H), 2.27 (d, 2H), 2.24 (s, 3H), 0.94-0.85 (m, 1H), 0.52-0.46 (m, 2H), 0.13-0.08 (m, 2H). |
| 313 | 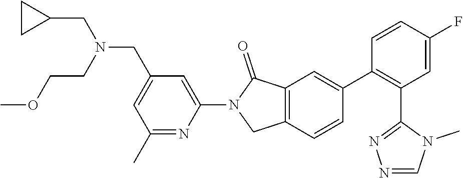 Starting materials: Compound 137a Reaction conditions: E″ | LCMS: 541.25 [M + H]⁺; ¹H-NMR (400 MHz, DMSO-d6): δ 8.45 (s, 1H), 8.34 (s, 1H), 7.75-7.72 (m, 1H), 7.68 (d, 1H), 7.63-7.58 (m, 1H), 7.53-7.50 (m, 2H), 7.39 (dd,1H), 7.03 (s,1H), 5.08 (s, 2H), 3.72 (s, 2H), 3.47 (t, 2H) 3.23 (s, 3H), 3.14 (s, 3H), 2.73 (t, 2H), 2.46 (s, 3H), 2.38 (d, 2H), 0.92-0.84 (m, 1H), 0.47-0.42 (m, 2H), 0.10- 0.06 (m, 2H). |
| 314 | 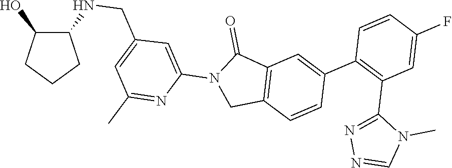 [SMILES: Cc1cc(CN[C@@H]2CCC[C@H]2O)cc(N2Cc3ccc(-c4ccc(F)cc4-c4nncn4C)cc3C2=O)n1] Starting materials: Compound 137a Reaction conditions: E″ | LCMS: 513.2 [M + H]⁺; ¹H-NMR (400 MHz, DMSO-d6): δ 8.45 (s, 1H), 8.31 (s, 1H), 7.73 (dd, 1H), 7.64 (d, 1H), 7.58 (td, 1H), 7.54-7.50 (m, 2H), 7.39 (dd, 1H), 7.05 (s, 1H), 5.09 (s, 2H), 4.51 (d, 1H), 3.79-3.76 (m, 1H), 3.75 (s, 2H), 3.15 (s, 3H), 2.80-2.73 (m, 1H), 2.46 (s, 3H), 1.89-1.78 (m, 2H), 1.63-1.52 (m, 2H), 1.45-1.25 (m, 2H). |
| 315 | 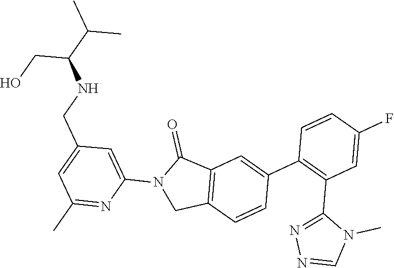 [SMILES: Cc1cc(CN[C@@H](CO)C(C)C)cc(N2Cc3ccc(-c4ccc(F)cc4-c4nncn4C)cc3C2=O)n1] Starting materials: Compound 137a Reaction conditions: E″ | LCMS: 515.2 [M + H]⁺; ¹H-NMR (400 MHz, DMSO-d6): δ 8.44 (s, 1H), 8.35 (s, 1H), 7.73 (dd, 1H), 7.64 (d, 1H), 7.58 (td, 1H), 7.54-7.50 (m, 2H), 7.38 (dd, 1H), 7.06 (s, 1H), 5.09 (s, 2H), 4.42 (t, 1H), 3.78 (ABq, 2H), 3.50- 3.44 (m, 1H), 3.40-3.34 (m, 1H), 3.14 (s, 3H), 2.46 (s, 3H), 2.30-2.25 (m, 1H), 1.85-1.75 (m, 1H), 0.90 (d, 3H), 0.88 (d, 3H). |
| 316 | 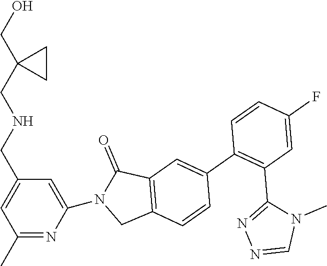 [SMILES: Cc1cc(CNCC2(CO)CC2)cc(N2Cc3ccc(-c4ccc(F)cc4-c4nncn4C)cc3C2=O)n1] Starting materials: Compound 137a Reaction conditions: E″ | LCMS: 513.15 [M + H]⁺; ¹H-NMR (400 MHz, DMSO-d6): δ 8.44 (s, 1H), 8.30 (s, 1H), 7.73 (dd, 1H), 7.64 (d, 1H), 7.58 (td, 1H), 7.54-7.50 (m, 2H), 7.38 (dd, 1H), 7.04 (s, 1H), 5.08 (s, 2H), 3.74 (s, 2H), 3.37 (s, 2H), 3.14 (s, 3H), 2.46 (s, 3H), 0.37 0.30 (m, 4H). |
| 317 | 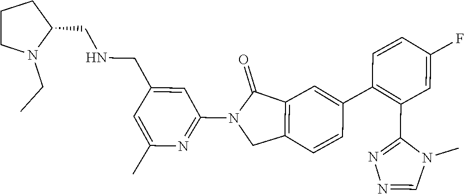 [SMILES: CCN1CCC[C@@H]1CNCc1cc(C)nc(N2Cc3ccc(-c4ccc(F)cc4-c4nncn4C)cc3C2=O)c1] Starting materials: Compound 137a Reaction conditions: E″ | LCMS: 540.2 [M + H]⁺; ¹H-NMR (400 MHz, CD3OD): δ 8.39 (s, 1H), 8.37 (s, 1H), 7.75 (dd, 1H), 7.66-7.64 (m, 2H), 7.57-7.45 (m, 3H), 7.06 (s, 1H), 5.16 (s, 2H), 3.89 (s, 2H), 3.60-3.50 (bm, 1H), 3.45-3.33 (bm, 2H), 3.20 (s, 3H), 3.02-2.91 (bm, 2H), 2.90- 2.82 (m, 2H), 2.53 (s, 3H), 2.28-2.21 (m, 1H), 2.12-1.93 (m, 2H), 1.85- 1.78 (m, 1H), 1.33 (t, 3H). |
| 318 | 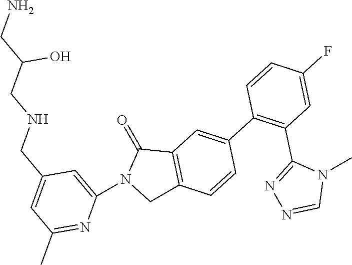 [SMILES: Cc1cc(CNCC(O)CN)cc(N2Cc3ccc(-c4ccc(F)cc4-c4nncn4C)cc3C2=O)n1] Starting materials: Compound 137a Reaction conditions: E″ | LCMS: 502.4 [M + H]⁺; ¹H-NMR (400 MHz, CD3OD): δ 8.40 (s, 1H), 8.33 (s, 1H), 7.75 (dd, 1H), 7.68 (d, 1H), 7.64 (d, 1H), 7.56 (td, 1H), 7.50-7.45 (m, 2H), 7.07 (s, 1H), 5.16 (s, 2H), 3.91- 3.79 (m, 3H), 3.20 (s, 3H), 2.95-2.87 (m, 1H), 2.81-2.70 (m, 2H), 2.69- 2.64 (m, 1H), 2.64 (s, 3H). |
| 319 | 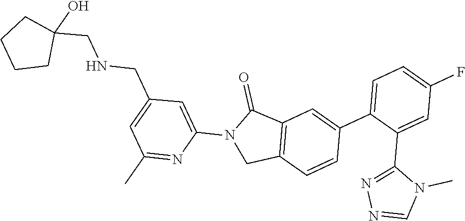 [SMILES: Cc1cc(CNCC2(O)CCCC2)cc(N2Cc3ccc(-c4ccc(F)cc4-c4nncn4C)cc3C2=O)n1] Starting materials: Compound 137a Reaction conditions: E″ | LCMS: 527.3 [M + H]⁺; ¹H-NMR (400 MHz, DMSO-d6): δ 8.44 (s, 1H), 8.32 (s, 1H), 7.73 (dd, 1H), 7.64 (d, 1H), 7.58 (td, 1H), 7.54-7.50 (m, 2H), 7.38 (dd, 1H), 7.05 (s, 1H), 5.08 (s, 2H), 4.20 (s, 1H), 3.78 (s, 2H), 3.44 (s, 2H), 3.14 (s, 3H), 2.46 (s, 3H), 1.73-1.63 (m, 2H), 1.62-1.42 (m, 6H). |
| 320 | 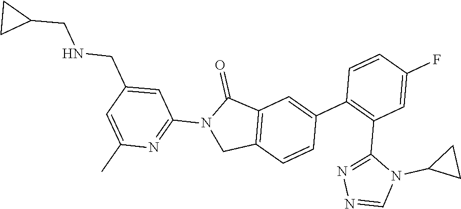 [SMILES: Cc1cc(CNCC2CC2)cc(N2Cc3ccc(-c4ccc(F)cc4-c4nncn4C4CC4)cc3C2=O)n1] Starting materials: Int-151 and Int-138 Reaction conditions: C″ | LCMS: 509.2 [M + H]⁺; 1H NMR (400 MHz, DMSO-d6): δ 8.48 (s, 1H), 8.31 (s, 1H), 7.73 (dd, 1H), 7.65 (d, 1H), 7.58 (td, 1H), 7.54-7.49 (m, 2H), 7.42 (dd, 1H), 7.04 (s, 1H), 5.09 (s, 2H), 3.76 (s, 2H), 3.15 (s, 3H), 2.48-2.46 (m, 1H), 2.45 (s, 3H), 2.39 (d, 2H), 0.97-0.85 (m, 1H), 0.71-0.59 (m, 4H), 0.45-0.38 (m, 2H), 0.15-0.08 (m, 2H). |
| 321 | 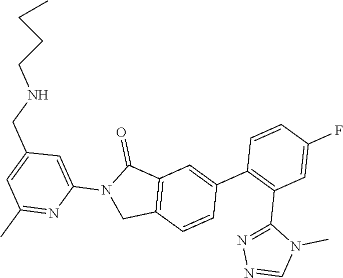 [SMILES: CCCCNCc1cc(C)nc(N2Cc3ccc(-c4ccc(F)cc4-c4nncn4C)cc3C2=O)c1] Starting materials: Compound 137a Reaction conditions: E″ | LCMS: 485.2 [M + H]⁺; 1H NMR (400 MHz, DMSO-d6): δ 8.44 (s, 1H), 8.31 (s, 1H), 7.73 (dd, 1H), 7.64 (d, 1H), 7.58 (td, 1H), 7.54-7.49 (m, 2H), 7.39 (dd, 1H), 7.04 (s, 1H), 5.08 (s, 2H), 3.72 (s, 2H), 3.15 (s, 3H), 2.45 (s, 3H), 1.47-1.39 (m, 2H), 1.38-1.29 (m, 2H), 0.88 (t, 3H). |
| 322 | 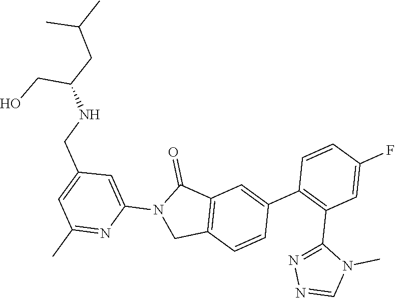 [SMILES: Cc1cc(CN[C@H](CO)CC(C)C)cc(N2Cc3ccc(-c4ccc(F)cc4-c4nncn4C)cc3C2=O)n1] Starting materials: Compound 137a Reaction conditions: E″ | LCMS: 529.2 [M + H]⁺; 1H NMR (400 MHz, DMSO-d6): δ 8.44 (s, 1H), 8.32 (s, 1H), 7.73 (dd, 1H), 7.64 (d, 1H), 7.58 (td, 1H), 7.54-7.49 (m, 2H), 7.38 (dd, 1H), 7.06 (s, 1H), 5.08 (s, 2H), 4.46 (t, 1H), 3.82-3.74 (m, 2H), 3.45 - 3.39 (m, 1H), 3.33-3.29 (m, 1H), 3.15 (s, 3H), 2.46 (s, 3H), 1.80-1.70 (m, 1H), 1.31-1.15 (m, 3H), 0.87 (d, 3H), 0.82 (d, 3H). |
| 323 | 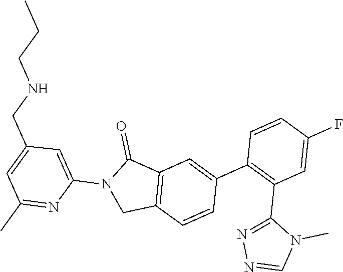 [SMILES: CCCNCc1cc(C)nc(N2Cc3ccc(-c4ccc(F)cc4-c4nncn4C)cc3C2=O)c1] Starting materials: Compound 137a Reaction conditions: E″ | LCMS: 471.2 [M + H]⁺; 1H NMR (400 MHz, DMSO-d6): δ 8.44 (s, 1H), 8.31 (s, 1H), 7.73 (dd, 1H), 7.64 (d, 1H), 7.58 (td, 1H), 7.54-7.49 (m, 2H), 7.38 (dd, 1H), 7.04 (s, 1H), 5.08 (s, 2H), 3.72 (s, 2H), 3.14 (s, 3H), 2.47-2.44 (m, 5H), 1.51-1.40 (m, 2H), 0.89 (t, 3H). |
| 324 | 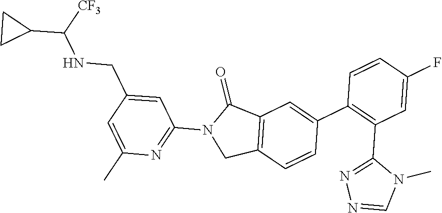 [SMILES: Cc1cc(CNC(C2CC2)C(F)(F)F)cc(N2Cc3ccc(-c4ccc(F)cc4-c4nncn4C)cc3C2=O)n1] Starting materials: Compound 137a Reaction conditions: E″ | LCMS: 551.15 [M + H]⁺; 1H NMR (400 MHz, DMSO-d6): δ 8.45 (s, 1H), 8.33 (s, 1H), 7.73 (dd, 1H), 7.64 (d, 1H), 7.58 (td, 1H), 7.54-7.49 (m, 2H), 7.38 (dd, 1H), 7.06 (s, 1H), 5.08 (s, 2H), 3.91 (d, 2H), 3.15 (s, 3H), 2.93-2.88 (m, 1H), 2.61-2.53 (m, 1H), 2.46 (s, 3H), 0.98-0.89 (m, 1H), 0.58-0.54 (m, 2H), 0.41-0.33 (m, 2H). |
| 325 | 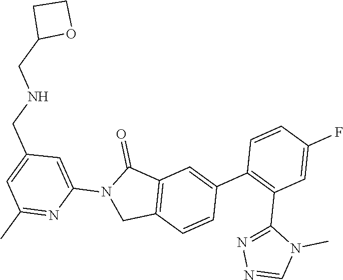 [SMILES: Cc1cc(CNCC2CCO2)cc(N2Cc3ccc(-c4ccc(F)cc4-c4nncn4C)cc3C2=O)n1] Starting materials: Compound 137a Reaction conditions: E″ | LCMS: 499.2 [M + H]⁺; ¹H-NMR (400 MHz, DMSO-d6): δ 8.44 (s, 1H), 8.33 (s, 1H), 7.73 (dd, 1H), 7.64 (d, 1H), 7.58 (td, 1H), 7.53-7.49 (m, 2H), 7.38 (dd, 1H), 7.04 (s, 1H), 5.09 (s, 2H), 4.84-4. 78 (m, 1H), 4.55-4.48 (m, 1H), 4.44-4.38 (m, 1H), 3.79 (s, 2H), 3.14 (s, 3H), 2.78-2.70 (m, 2H), 2.60 - 2.55 (m, 1H), 2.46 (s, 3H), 2.42- 2.38 (m, 1H). |
| 326 | 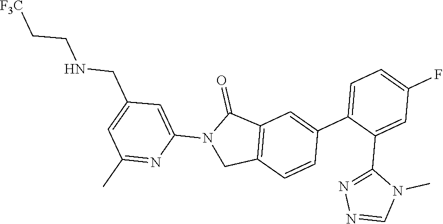 [SMILES: Cc1cc(CNCCC(F)(F)F)cc(N2Cc3ccc(-c4ccc(F)cc4-c4nncn4C)cc3C2=O)n1] Starting materials: Compound 137a Reaction conditions: E″ | LCMS: 525.1 [M + H]⁺; ¹H-NMR (400 MHz, CD3OD): δ 8.39 (s, 1H), 8.33 (s, 1H), 7.75 (dd, 1H), 7.71-7.70 (m, 1H), 7.62 (dd, 1H), 7.56 (td, 1H), 7.48 - 7.43 (m, 2H), 7.07 (s, 1H), 5.16 (s, 2H), 3.85 (s, 2H), 3.20 (s, 3H), 2.88 (dd, 2H), 2.53 (s, 3H), 2.49-2.39 (m, 2H). |
| 327 | 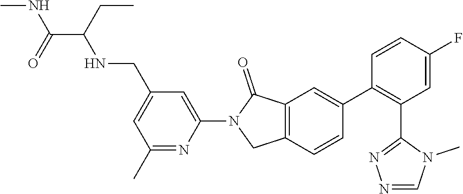 Starting materials: Compound 137a Reaction conditions: E″ | LCMS: 528.2 [M + H]⁺; ¹H-NMR (400 MHz, DMSO-d6): δ 8.45 (s, 1H), 8.32 (s, 1H), 7.85-7.78 (m, 1H), 7.73 (dd, 1H), 7.64 (d, 1H), 7.58 (td, 1H), 7.55- 7.49 (m, 2H), 7.38 (dd,1H), 7.04 (s, 1H), 5.08 (s, 2H), 3.74 (d, 1H), 3.53 (d, 1H), 3.15 (s, 3H), 2.89 (t, 1H), 2.63 (d, 2H), 2.45 (s, 3H), 1.58-1.48 (m, 2H), 0.87 (t, 3H). |
| 328 | 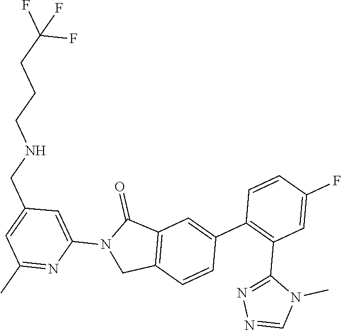 [SMILES: Cc1cc(CNCCCC(F)(F)F)cc(N2Cc3ccc(-c4ccc(F)cc4-c4nncn4C)cc3C2=O)n1] Starting materials: Compound 137a Reaction conditions: E″ | LCMS: 539.2 [M + H]⁺; ¹H-NMR (400 MHz, CD3OD): δ 8.40 (s, 1H), 8.32 (s, 1H), 7.75 (dd, 1H), 7.71 (d, 1H), 7.62 (d, 1H), 7.54 (td, 1H), 7.48-7.43 (m, 2H), 7.07 (s, 1H), 5.16 (s, 2H), 3.83 (s, 2H), 3.22 (s, 3H), 2.70 (t, 2H), 2.53 (s, 3H), 2.33-2.18 (m, 2H), 1.85-1.75 (m, 2H). |
| 329 | 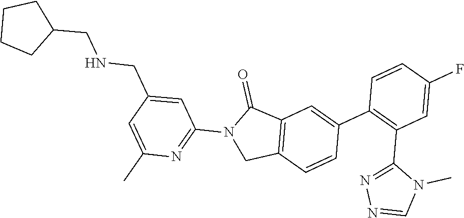 [SMILES: Cc1cc(CNCC2CCCC2)cc(N2Cc3ccc(-c4ccc(F)cc4-c4nncn4C)cc3C2=O)n1] Starting materials: Compound 137a Reaction conditions: E″ | LCMS: 511.2 [M + H]⁺; ¹H-NMR (400 MHz, DMSO-d6): δ 8.44 (s, 1H), 8.33 (s, 1H), 7.73 (dd, 1H), 7.64 (d, 1H), 7.58 (td, 1H), 7.53-7.50 (m, 2H), 7.39 (dd, 1H), 7.05 (s, 1H), 5.08 (s, 2H), 3.77 (s, 2H), 3.15 (s, 3H), 2.46 (brs, 5H), 2.05-1.95 (m, 1H), 1.78-1.67 (m, 2H), 1.60-1.42 (m, 4H), 1.24- 1.12 (m, 2H). |
| 330 | 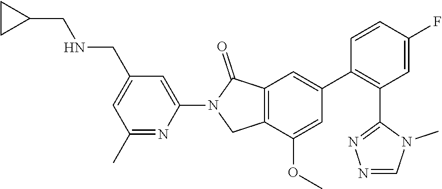 [SMILES: COc1cc(-c2ccc(F)cc2-c2nncn2C)cc2c1CN(c1cc(CNCC3CC3)cc(C)n1)C2=O] Starting materials: Int-151 and Int-145 Reaction conditions: C″ | LCMS: 513.2 [M + H]⁺; 1H NMR (400 MHz, DMSO-d6): δ 8.47 (s, 1H), 8.28 (s, 1H), 7.80 (dd, 1H), 7.59 (td, 1H), 7.52 (dd, 1H), 7.17 (d, 1H), 7.04 (s, 1H), 6.88 (d, 1H), 4.95 (s, 2H), 3.76 (s, 2H), 3.75 (s, 3H), 3.13 (s, 1H), 2.45 (s, 3H), 2.39 (d, 2H), 0.98-0.86 (m, 1H), 0.43-0.38 (m, 2H), 0.14-0.09 (m, 2H). |

### Example 21. 2-(4-(Aminomethyl)-6-methylpyridin-2-yl)-6-(4-fluoro-2-(4-methyl-4H-1,2,4triazol-3-yl)phenyl)isoindolin-1-one (Compound 331)

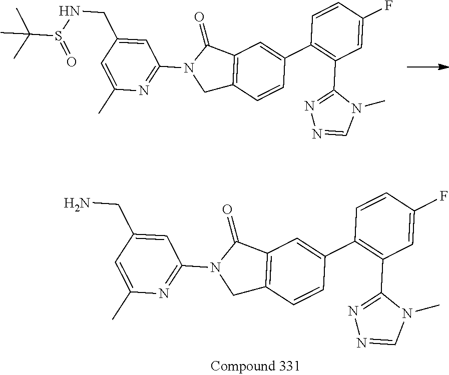 [SMILES: Cc1cc(CNS(=O)C(C)(C)C)cc(N2Cc3ccc(-c4ccc(F)cc4-c4nncn4C)cc3C2=O)n1 ; Cc1cc(CN)cc(N2Cc3ccc(-c4ccc(F)cc4-c4nncn4C)cc3C2=O)n1]

To a solution of N-((2-(6-(4-fluoro-2-(4-methyl-4H-1,2,4-triazol-3-yl)phenyl)-1-oxoisoindolin-2-yl)-6-methylpyridin-4-yl)methyl)-2-methylpropane-2-sulfinamide (0.29 g, 0.54 mmol) in 1,4-dioxane was added 4 N HCl in 1,4-dioxane (0.54 mL, 2.16 mmol) at 0° C. followed by stirring at RT for 2 h. The mixture was concentrated and purified by prep HPLC using water and acetonitrile as eluent to obtain the title compound as white solid (0.060 g, 25.74%); LCMS: 429.2 [M+H]⁺. ¹H-NMR (400 MHz, DMSO-d6): δ 8.44 (s, 1H), 8.29 (s, 1H), 7.73 (dd, 1H), 7.64 (d, 1H), 7.58 (td, 1H), 7.54-7.50 (m, 2H), 7.39 (dd, 1H), 7.06 (s, 1H), 5.08 (s, 2H), 3.76 (s, 2H), 3.15 (s, 3H), 2.45 (s, 3H).

### Example 22. 6-(4-Fluoro-2-(4-methyl-4H-1,2,4-triazol-3-yl)phenyl)-2-(6-methyl-4-(1-(1-methyl-1H-pyrazol-4-yl)vinyl)pyridin-2-yl)isoindolin-1-one (Compound 332) and 6-(4-Fluoro-2-(4-methyl-4H-1,2,4-triazol-3-yl)phenyl)-2-(6-methyl-4-(1-(1-methyl-1H-pyrazol-4-yl)ethyl)pyridin-2-yl)isoindolin-1-one (Compound 333)

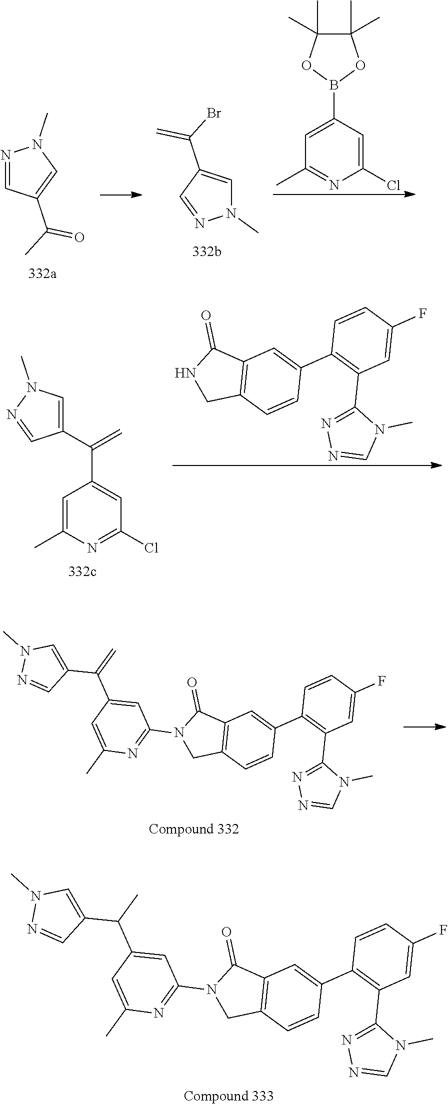

### a) 4-(1-Bromovinyl)-1-methyl-1H-pyrazole (332b)

To a solution of 1-(1-methyl-1H-pyrazol-4-yl) ethan1-one (5.0 g, 40.28 mmol) in DCM (100.0 mL) was added triethylamine (8.5 mL, 61.09 mmol) and triphenyl phosphite (12.5 g, 40.27 mmol) at −78° C. followed by dropwise addition of bromine (3.86 g, 48.33 mmol). The solution was stirred at RT for 12 h. The reaction was quenched with aqueous sodium bicarbonate solution and extracted with ethyl acetate.

The combined organic layer was dried over sodium sulphate, filtered, concentrated, and purified by silica-gel column chromatography on silica using 20-40% ethyl acetate in hexane as eluent to afford the title compound (2.0 g, 26.55%). LCMS: 187.0 [M+H]⁺.

### b) 2-Chloro-6-methyl-4-(1-(1-methyl-1H-pyrazol-4-yl) vinyl) pyridine (332c)

To a degassed solution of 2-chloro-6-methyl-4-(4,4,5,5-tetramethyl-1,3,2-dioxaborolan-2-yl)pyridine (2.0 g, 7.89 mmol), compound 332b (1.92 g, 10.26 mmol), and K₂CO₃ (3.27 g, 23.66 mmol) in dioxane (20.0 mL) and water (5.0 mL) was added Pd(amphos)Cl₂ (0.56 g, 0.78 mmol) followed by stirring at 80° C. for 6 h. The mixture was filtered through celite bed followed by washing with ethyl acetate. The filtrated was extracted with water. The combined organic layer was dried over sodium sulphate, filtered, concentrated, and purified by silica-gel flash column chromatography using 20-40% ethyl acetate in hexane as eluent to obtain the desired product (1.6 g, 86.78%). LCMS: 234.1 [M+H]⁺.

### c) 6-(4-Fluoro-2-(4-methyl-4H-1,2,4-triazol-3-yl)phenyl)-2-(6-methyl-4-(1-(1-methyl-1H-pyrazol-4-yl)vinyl)pyridin-2-yl)isoindolin-1-one (Compound 332)

The compound was prepared according to the procedure described for Example-17 with appropriate variations in reactants, quantities of reagents, and solvents. LCMS: 506.2 [M+H]⁺. ¹H-NMR (400 MHz, CDCl₃): δ 8.50 (s, 1H), 8.02 (s, 1H), 7.93 (d, 1H), 7.62-7.58 (m, 2H), 7.43-7.38 (m, 4H), 7.29 (dd, 1H), 7.00 (s, 1H), 5.56 (s, 1H), 5.40 (s, 1H), 5.16 (s, 2H), 3.94 (s, 3H), 3.02 (s, 3H), 2.55 (s, 3H).

### d) 6-(4-Fluoro-2-(4-methyl-4H-1,2,4-triazol-3-yl)phenyl)-2-(6-methyl-4-(1-(1-methyl-1H-pyrazol-4-yl)ethyl)pyridin-2-yl)isoindolin-1-one (Compound 333)

To a solution of compound 332 (0.07 g, 0.138 mmol) in EtOH (2 mL) was added Pd on carbon (10% weight) (0.029 g, 0.27 mmol) followed by stirring the mixture under hydrogen at RT for 3 h. The mixture was filtered through celite bed followed by washing with EtOH. The filtrate was concentrated and triturated with pentane to afford the title compound as white solid (0.055 g, 78.5%). LCMS: 508.2 [M+H]⁺. ¹H-NMR (400 MHz, CDCl₃): δ 8.38 (s, 1H), 8.01 (s, 1H), 7.92 (s, 1H), 7.61 (dd, 1H), 7.42-7.35 (m, 4H), 7.27 (dd, 1H), 7.20 (s, 1H), 6.80 (s, 1H), 5.12 (s, 2H), 4.07 (q, 1H), 3.88 (s, 3H), 3.00 (s, 3H), 2.49 (s, 3H), 1.63 (d, 3H).

### Example 23. 6-(4-Fluoro-2-(4-methyl-4H-1,2,4-triazol-3-yl)phenyl)-2-(6-methyl-4-(1-(1-methyl-1H-imidazol-2-yl)ethyl)pyridin-2-yl)isoindolin-1-one (Compound 334)

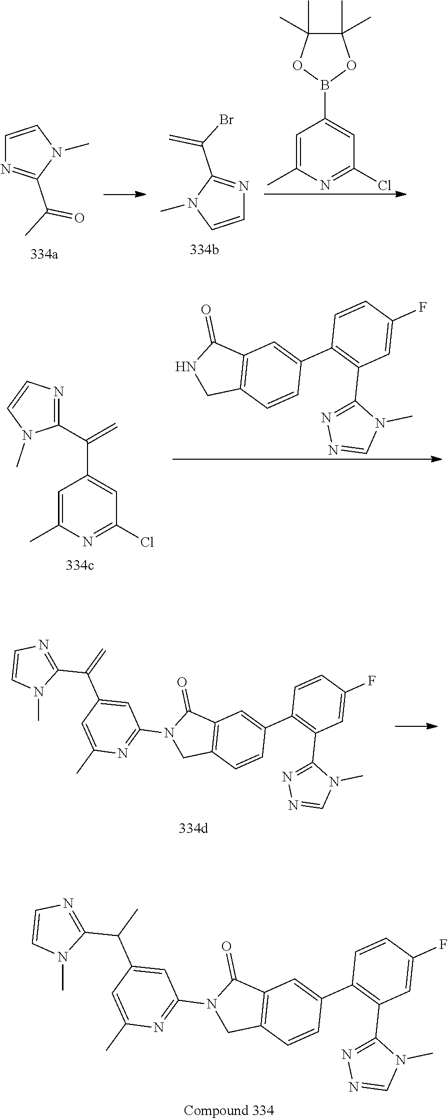

### a) 2-(1-Bromovinyl)-1-methyl-1H-imidazole (334b)

The compound was prepared according to the procedure described for compound 332b with appropriate variations in reactants, quantities of reagents, and solvents. LCMS: 187.0 [M+H]⁺.

### b) 2-Chloro-6-methyl-4-(1-(1-methyl-1H-imidazol-2-yl)vinyl)pyridine (334c)

The compound was prepared according to the procedure described for compound 332c with appropriate variations in reactants, quantities of reagents, and solvents. LCMS: 234.1 [M+H]⁺.

### c) 6-(4-Fluoro-2-(4-methyl-4H-1,2,4-triazol-3-yl)phenyl)-2-(6-methyl-4-(1-(1-methyl-1H-imidazol-2-yl)vinyl)pyridin-2-yl)isoindolin-1-one (334d)

The compound was prepared according to the procedure described compound 332 with appropriate variations in reactants, quantities of reagents, and solvents. LCMS: 506.2 [M+H]⁺.

### d) 6-(4-Fluoro-2-(4-methyl-4H-1,2,4-triazol-3-yl)phenyl)-2-(6-methyl-4-(1-(1-methyl-1H-imidazol-2-yl)ethyl)pyridin-2-yl)isoindolin-1-one (Compound 334)

The compound was prepared according to the procedure described for compound 333 with appropriate variations in reactants, quantities of reagents, and solvents. LCMS: 508.2 [M+H]⁺. 1H-NMR (400 MHz, DMSO-d6): δ 8.44 (s, 1H), 8.19 (s, 1H), 7.72 (dd, 1H), 7.64 (d, 1H), 7.59 (td, 1H), 7.52 (dd, 1H), 7.47 (d, 1H), 7.40 (dd, 1H), 7.05 (d, 1H), 6.86 (d, 1H), 6.85 (d, 1H), 5.06 (s, 2H), 4.34 (q, 1H), 3.43 (s, 3H), 3.13 (s, 3H), 2.42 (s, 3H), 1.59 (d, 3H).

### Example 24. (Z)-2-(6-Chloro-4-((1-methylpiperidin-3-ylidene)methyl)pyridin-2-yl)-6-(3-((4-methyl-4H-1,2,4-triazol-3-yl)methyl)oxetan-3-yl)isoindolin-1-one (Compound 335)

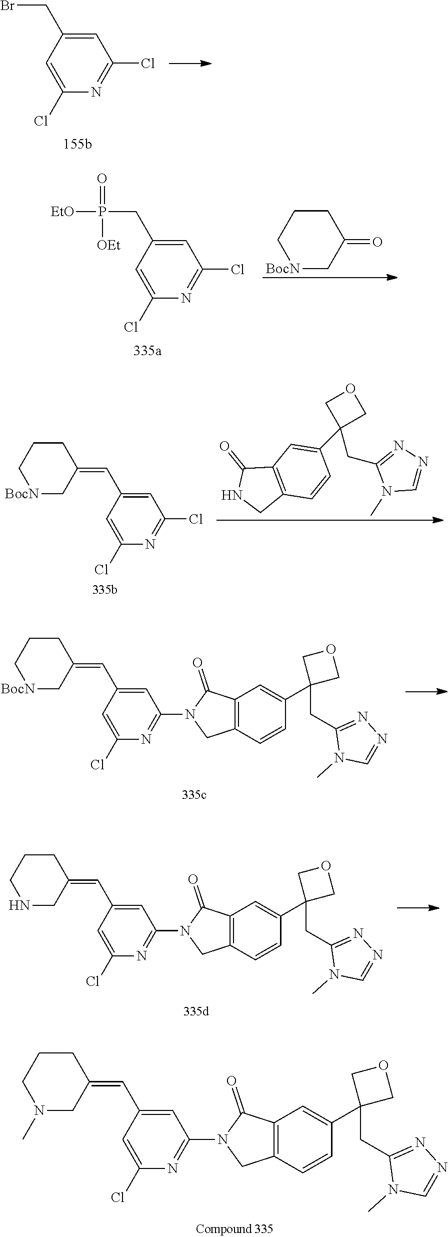

### a) Diethyl ((2,6-dichloropyridin-4-1)methyl)phosphonate (335a)

To the compound 4-(bromomethyl)-2,6-dichloropyridine (2.0 g, 8.30 mmol) was added triethyl phosphite (12 mL) followed by heating to 80° C. for 12 h. After 12 h the mixture was concentrated to afford the title compound (2.4 g) as crude product which was used in next step as such. LCMS: 298.0 [M+H]⁺.

### b) tert-Butyl (Z)-3-((2,6-dichloropyridin-4-yl)methylene)piperidine-1-carboxylate (335b)

To a solution of diisopropylamine (1.07 g, 10.60 mmol) in dry THF was added n-butyl lithium (0.67 g, 10.60 mmol) at −78° C. followed by stirring for 1 h. The resultant light-yellow solution was added to compound 335a (1.89 g, 6.36 mmol) in dry THF followed by stirring for 1 h. Then tert-butyl 3-oxopiperidine-1-carboxylate (0.84 g, 4.24 mmol) was added to the mixture followed by stirring for 3 h. The mixture was quenched with saturated aqueous ammonium chloride solution and extracted with ethyl acetate. The combined organic layer was dried over anhydrous sodium sulphate, filtered, concentrated, and purified by silica-gel flash column chromatography using 10-30% ethyl acetate in hexane as eluent to afford the title compound (1.0 g, 68.69%). LCMS: 343.1[M+H]⁺

### c) tert-Butyl (Z)-3-((2-chloro-6-(6-(3-((4-methyl-4H-1,2,4-triazol-3-yl)methyl)-oxetan-3-yl)-1-oxoisoindolin-2-yl)pyridin-4-yl)methylene)piperidine-1-carboxylate (335c)

To a degassed solution of compound 335b (0.19 g, 0.56 mmol), 6-(3-((4-methyl-4H-1,2,4-triazol-3-yl)methyl)oxetan-3-yl)isoindolin-1-one (016 g, 0.56 mmol), cesium carbonate (0.36 g, 1.12 mmol) and Xantphos (0.06 g, 0.11 mmol) in DMSO (4.0 ml) was added Pd(OAc)₂ (0.01 g, 0.05 mmol) followed by heating at 90° C. for 3 h. The mixture was cooled to RT, passed through celite bed followed by washing the celite pad with 10% methanol in DCM. The filtrate was diluted with water and extracted with 10% methanol in DCM. The combined organic layer was washed with brine, dried over anhydrous sodium sulphate, filtered, concentrated, and purified by silica-gel flash column chromatography using 8% MeOH in DCM as eluent to afford the title compound (0.15 g, 45.07%). LCMS: 591.4 [M+H]⁺.

### d) (Z)-2-(6-Chloro-4-(piperidin-3-ylidenemethyl)pyridin-2-yl)-6-(3-((4-methyl-4H-1,2,4-triazol-3-yl)methyl)oxetan-3-yl)isoindolin-1-one (335d)

To a stirred solution of compound 335c (0.13 g, 0.22 mmol) in DCM (1.7 ml) was added TFA (0.3 ml) followed by stirring at RT for 1 h. The mixture was concentrated, basified with saturated aqueous sodium bicarbonate solution and extracted with DCM. The combined organic layer was dried over anhydrous sodium sulphate, filtered, concentrated to afford the title compound (0.11 g) as crude product which was used in next step as such. LCMS: 491.3 [M+H]⁺.

### e) (Z)-2-(6-Chloro-4-((1-methylpiperidin-3-ylidene)methyl)pyridin-2-yl)-6-(3-((4-methyl-4H-1,2,4-triazol-3-yl)methyl)oxetan-3-yl)isoindolin-1-one (Compound 335)

To a solution of compound 335d (0.10 g, 0.20 mmol) and formaldehyde (0.06 g, 2.04 mmol) in MeOH was added formic acid (0.03 g, 0.81 mmol) and heated at 70° C. for 6 h. The mixture was concentrated, basified with saturated aqueous sodium bicarbonate solution and extracted with ethyl acetate. The combined organic layer was dried over anhydrous sodium sulphate, filtered, concentrated, and purified by preparative TLC method using 8% MeOH-DCM as eluent to afford the title compound (0.029 g, 2.81%). LCMS: 505.20 [M+H]⁺. ¹HNMR (400 MHz, DMSO-d6): δ 8.37 (d, 1H), 8.21 (s, 1H), 7.62 (d, 1H), 7.49 (s, 1H), 7.36 (dd, 1H), 7.13 (s, 1H), 5.58 (s, 1H), 5.02 (s, 2H), 4.98 (d, 2H), 4.90 (d, 2H), 3.57 (s, 2H), 3.34 (s, 2H), 3.01 (s, 3H), 2.70-2.65 (m, 3H), 2.38-2.35 (m, 2H), 2.12 (bm, 2H), 2.01-1.99 (m, 2H).

### Example 25. 2-(6-Methyl-4-((pyrrolidin-2-ylmethyl)amino)pyridin-2-yl)-6-(3-((4-methyl-4H-1,2,4-triazol-3-yl)methyl)oxetan-3-yl)isoindolin-1-one (Compound 336)

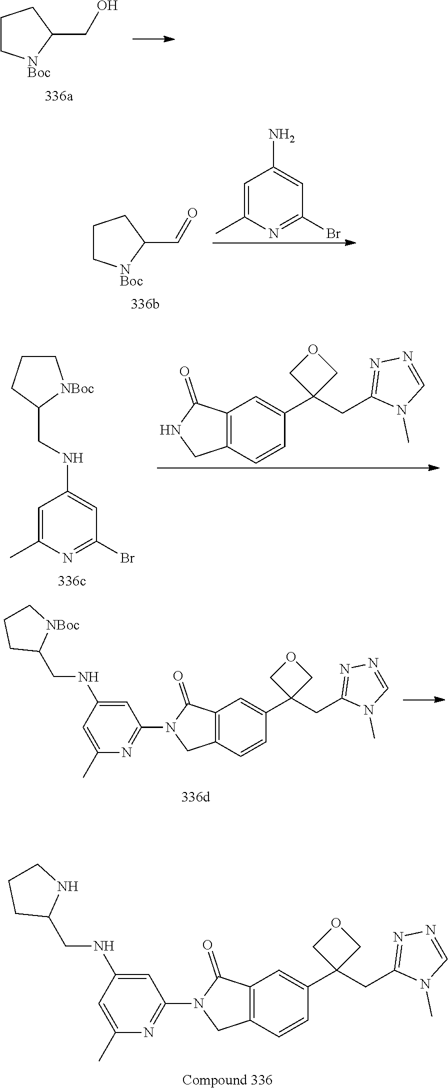

### a) tert-Butyl 2-formylpyrrolidine-1-carboxylate (336b)

To a stirred suspension of oxalyl chloride (15.76 g, 124.21 mmol) and 3 A° molecular sieves in DCM (125.0 mL) was added DMSO (19.40 g, 248.42 mmol) in DCM (25.0 ml) at −78° C. tert-Butyl 2-(hydroxymethyl)pyrrolidine-1-carboxylate (5.0 g, 24.84 mmol) in DCM (75.0 mL) was added dropwise followed by stirring for 30 min. Triethylamine (37.70 g, 372.6 mmol) was added to the mixture dropwise followed by stirring for 1 h at −78° C. The mixture was slowly warmed to RT and stirred for 12 h. The reaction mixture was quenched by saturated aqueous ammonium chloride solution and extracted with ethyl acetate. The combined organic layer was dried over anhydrous sodium sulphate, filtered, concentrated, and purified through silica-gel flash column chromatography using 10-20% ethyl acetate in hexane as eluent to afford the title compound (4.0 g, 80%). LCMS: 144 [M−56H]⁺.

### b) tert-Butyl 2-(((2-bromo-6-methylpyridin-4-yl) amino)methyl)pyrrolidine-1-carboxylate (336c)

To a solution of 2-bromo-6-methylpyridin-4-amine (1.10 g, 5.88 mmol) and compound 336b (2.92 g, 14.7 mmol) in DCM (50.0 mL) was added acetic acid (0.5 ml) followed by stirring at RT for 12 h. STAB (3.11 g, 14.71 mmol) was added to the mixture followed by stirring at RT for 16 h. The reaction mixture was quenched with water and extracted with DCM. The combined organic layer was treated as in previous step using 15-20% ethyl acetate in hexane as eluent to afford the title compound (1.0 g, 45.92%). LCMS: 372.1 [M+H]⁺.

### c) tert-Butyl 2-(((2-methyl-6-(6-(3-((4-methyl-4H-1,2,4-triazol-3-yl)methyl)-oxetan-3-yl)-1-oxoisoindolin-2-yl)pyridin-4-yl)amino)methyl)pyrrolidine-1-carboxylate (336d)

The compound was prepared according to the procedure described for Compound 128 with appropriate variations in reactants, quantities of reagents. LCMS: 574.3 [M+H]⁺.

### e) 2-(6-Methyl-4-((pyrrolidin-2-ylmethyl)amino)pyridin-2-yl)-6-(3-((4-methyl-4H-1,2,4-triazol-3-yl)methyl)oxetan-3-yl)isoindolin-1-one (Compound 336)

To a solution of compound 336d (0.04 g, 0.49 mmol) in DCM (1.35 ml) was added TFA (0.45 ml) at 5° C. followed by stirring at RT for 16 h. The mixture was concentrated and purified by passing through bicarbonate resin using 5% water in ACN (5.0 ml) as eluent to afford the title compound (0.009 g, 24.36%). LCMS: 474.3 [M+H]⁺. ¹HNMR (400 MHz, DMSO-d6): δ 8.21 (s, 1H), 7.60-7.57 (m, 2H), 7.42 (d, 1H), 7.29 (dd, 1H), 6.55 (t, 1H), 6.25 (d, 1H), 4.98-4.96 (m, 4H), 4.90 (d, 2H), 3.56 (s, 2H), 3.25-3.18 (m, 1H), 3.05-2.97 (m, 5H), 2.84-2.73 (m, 2H), 2.27 (s, 3H), 1.85-1.75 (m, 1H), 1.75-1.58 (m, 2H), 1.40-1.30 (m, 1H).

### Example 26. 2-(4-(((Cyclopropyl methyl) amino) methyl)-6-methyl-5-(trifluoro-methyl) pyridin-2-yl)-6-(4-fluoro-2-(4-methyl-4H-1,2,4-triazol-3-yl)phenyl)isoindolin-1-one (Compound 337)

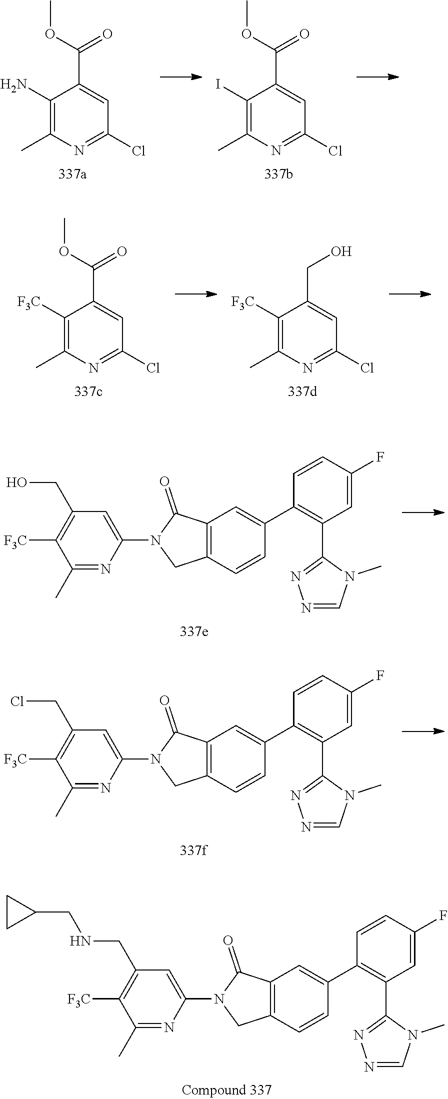

### a) Methyl 6-chloro-3-iodo-2-methylisonicotinate (337b)

To a solution of methyl 3-amino-6-chloro-2-methylisonicotinate (3.0 g, 14.95 mmol) (prepared according to WO2008/130021) in acetonitrile (12.5 mL) was added potassium iodide (12.41 g, 74.77 mmol), tert-butyl nitrite (7.71 g, 74.77 mmol) and copper iodide (3.41 g, 17.94 mmol) followed by heating at 80° C. for 12 h. The mixture cooled to RT, quenched with water, and extracted with ethyl acetate. The combined organic layer was dried over sodium sulphate, filtered, concentrated, and purified by silica-gel flash column chromatography using 0-10% ethyl acetate in hexane as eluent to obtain the title compound (1.7 g, 36.50%). LCMS: 311.9 [M+H]⁺.

### b) Methyl 6-chloro-2-methyl-3-(trifluoromethyl)isonicotinate (337c)

To a solution of compound 337b (0.60 g, 1.92 mmol), in DMF (3.0 mL), copper(I)iodide (0.55 g, 2.88 mmol) and methyl 2,2-difluoro-2-fluorosulfonylacetate (0.55 g, 2.88 mmol) was added followed by stirring at 70° C. for 12 h. The reaction mixture was quenched with brine and extracted with hexane. The combined organic layer was dried over sodium sulphate, filtered, concentrated to get the crude (0.45 g) which was used for the next step as such. LCMS: 254.0 [M+H]⁺.

### c) (6-Chloro-2-methyl-3-(trifluoromethyl) pyridin-4-yl) methanol (337d)

The compound was prepared according to the procedure described for Intermediate-151b with appropriate variations in reactants, quantities of reagents. LCMS: 226.0 [M+H]⁺.

### d) 6-(4-Fluoro-2-(4-methyl-4H-1,2,4-triazol-3-yl) phenyl)-2-(4-(hydroxy-methyl)-6-methyl-5-(trifluoromethyl) pyridin-2-yl) isoindolin-1-one (337e)

The compound was prepared according to the procedure described for Compound 136 with appropriate variations in reactants, quantities of reagents. LCMS: 498.1 [M+H]⁺.

### e) 2-(4-(Chloromethyl)-6-methyl-5-(trifluoromethyl) pyridin-2-yl)-6-(4-fluoro-2-(4-methyl-4H-1,2,4-triazol-3-yl) phenyl) isoindolin-1-one (337f)

The compound was prepared according to the procedure described for Compound 137a with appropriate variations in reactants, quantities of reagents. LCMS: 516.1 [M+H]⁺.

### f) 2-(4-(((Cyclopropyl methyl) amino) methyl)-6-methyl-5-(trifluoromethyl)-pyridin-2-yl)-6-(4-fluoro-2-(4-methyl-4H-1,2,4-triazol-3-yl)phenyl)isoindolin-1-one (Compound 337)

The compound was prepared according to the procedure described for Compound 137 with appropriate variations in reactants, quantities of reagents. LCMS: 551.0 [M+H]⁺. ¹H-NMR (400 MHz, DMSO): δ 8.85 (s, 1H), 8.45 (s, 1H), 7.74 (dd, 1H), 7.67 (d, 1H), 7.61-7.50 (m, 3H), 7.42 (dd, 1H), 5.12 (s, 2H), 3.93 (s, 2H), 3.17 (s, 3H), 2.68-2.61 (m, 3H), 2.44 (d, 2H), 0.98-0.90 (m, 1H), 0.45-0.40 (m, 2H), 0.20-0.12 (m, 2H).

### Example 27. 2-(4-(((1-Cyclopropyl-3-hydroxy-3-methylbutyl)amino)methyl)-6-methylpyridin-2-yl)-6-(4-fluoro-2-(4-methyl-4H-1,2,4-triazol-3-yl)phenyl)isoindolin-1-one (Compound 338)

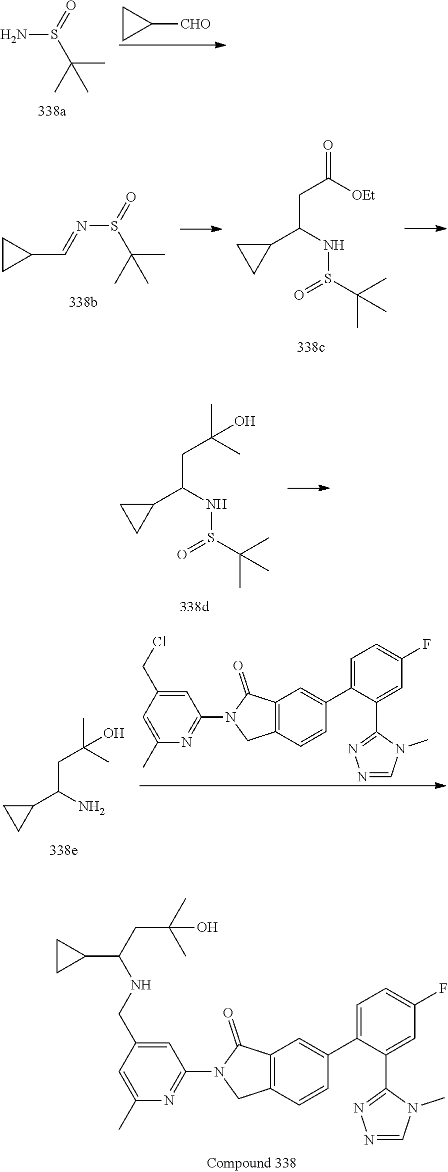

### a) (E)-N-(cyclopropylmethylene)-2-methylpropane-2-sulfinamide (338b)

To a solution of cyclopropanecarbaldehyde (5.0 g, 71.33 mmol) in THF (100.0 mL) was added 2-methyl-2-propanesulfinamide (8.64 g, 71.33 mmol) followed by heating to 40° C. for 14 h. The reaction mixture was quenched with brine and extracted with ethyl acetate. The combined organic layer was dried over sodium sulphate, filtered, concentrated, and purified by silica-gel flash column chromatography using 5-10% ethyl acetate in hexane as eluent to afford the title compound (2.0 g, 16.18%). LCMS: 174.1 [M+H]⁺.

### b) Ethyl 3-((tert-butylsulfinyl) amino)-3-cyclopropylpropanoate (338c)

To a stirred suspension of activated zinc (12.83 g, 196.22 mmol) in dry THF (17.0 ml) was added cooper(1) chloride (1.94 g, 19.62 mmol) under argon atmosphere followed by heating at 50° C. for 30 min. The resultant mixture was cooled to 0° C. Ethyl 2-bromoacetate (3.27g, 19.62 mmol) was added dropwise followed by heating the mixture at 50° C. for 30 min. The mixture was cooled to 0° C. and compound 338b in THF (8.0 mL) was added followed by stirring at RT for 14 h. The mixture was filtered through celite pad washed with ethyl acetate. The organic layer was washed with 0.25 M aqueous citric acid and with 1 M aqueous sodium bicarbonate. The combined organic layer was treated as in the previous step using 0-50% ethyl acetate in hexane as eluent to afford the title compound (1.7 g, 66.29%). LCMS: 262.2 [M+H]⁺.

### c) N-(1-Cyclopropyl-3-hydroxy-3-methylbutyl)-2-methylpropane-2-sulfinamide (338d)

To a solution of compound 338c (1.5 g, 5.74 mmol) in dry THF (20.0 mL) was added methyl magnesium bromide (3.0 M in diethyl ether (19.13 mL, 57.39 mmol) at RT followed by stirring for 14 h. The reaction mixture was quenched by saturated aqueous ammonium chloride solution at 0° C. and extracted with ethyl acetate. The combined organic layer was treated as in the previous step using using 40-50% ethyl acetate in hexane as eluent to afford the title compound (0.60 g, 42.27%). LCMS: 248.2 [M+H]⁺.

### d) 4-Amino-4-cyclopropyl-2-methylbutan-2-ol (338e)

To a solution of compound 338d (0.54 g, 2.18 mmol) in MeOH (1.35 mL) was added 4 M HCl in 1 4-dioxane (0.54 ml, 2.16 mmol) at 0° C. followed by stirring for 30 min at RT. The mixture was concentrated, neutralized by saturated NaHCO₃ solution, and extracted with DCM. The combined organic layer was dried over sodium sulphate, filtered and concentrated to get the solid. The solid was triturated with diethyl ether to afford the title compound (0.33 g) which was used in next step as such. LCMS: 144.2 [M+H]⁺.

### f) 2-(4-(((1-Cyclopropyl-3-hydroxy-3-methylbutyl) amino)methyl)-6-methyl-pyridin-2-yl)-6-(4-fluoro-2-(4-methyl-4H-1,2,4-triazol-3-yl)phenyl)isoindolin-1-one (Compound 338)

The compound was prepared according to the procedure described for Compound 137 with appropriate variations in reactants, quantities of reagents. LCMS: 555.2 [M+H]⁺. ¹H-NMR (400 MHz, DMSO-d6): δ 8.45 (s, 1H), 8.30 (s, 1H), 7.73 (dd, 1H), 7.64 (d, 1H), 7.59 (td, 1H), 7.54-7.50 (m, 2H), 7.39 (dd, 1H), 7.04 (s, 1H), 5.09 (s, 2H), 4.03 (d, 1H), 3.80 (d, 1H), 3.15 (s, 3H), 2.46 (s, 3H), 2.11-2.04 (m, 1H), 1.65 (dd, 1H), 1.55 (dd, 1H), 1.11 (s, 3H), 1.07 (s, 3H), 0.80-0.70 (m, 1H), 0.62-0.55 (m, 1H), 0.43-0.31 (m, 2H), −0.066-−0.012 (m, 1H).

### Example 28. 6-(4-Fluoro-2-(4-methyl-4H-1,2,4-triazol-3-yl)phenyl)-2-(6-methyl-4-(((piperidin-2-ylmethyl)amino)methyl)pyridin-2-yl)isoindolin-1-one (Compound 339)

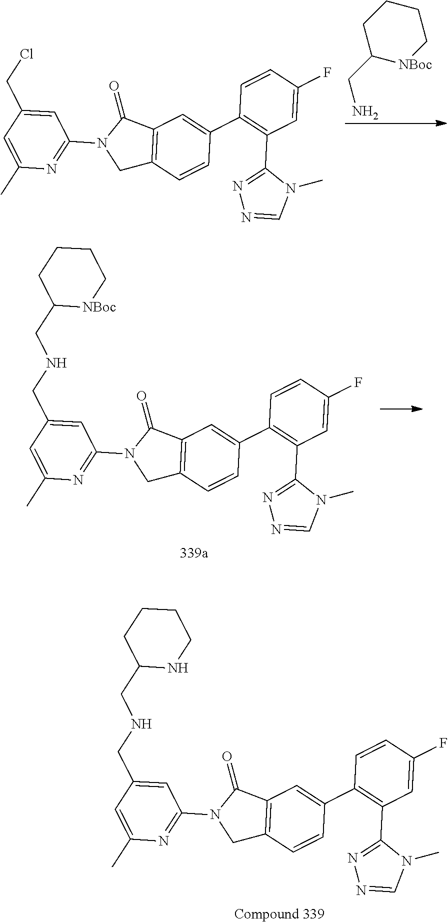

### a) tert-Butyl 2-((((2-(6-(4-fluoro-2-(4-methyl-4H-1,2,4-triazol-3-yl)phenyl)-1-oxoisoindolin-2-yl)-6-methylpyridin-4-yl)methyl)amino)methyl)piperidine-1-carboxylate (339a)

The compound was prepared according to the procedure described for compound 137 with appropriate variations in reactants, quantities of reagents. LCMS: 626.20 [M+H]⁺.

### b) 6-(4-Fluoro-2-(4-methyl-4H-1,2,4-triazol-3-yl)phenyl)-2-(6-methyl-4-(((piperidin-2-ylmethyl)amino)methyl)pyridin-2-yl)isoindolin-1-one (Compound 339)

The compound was prepared according to the procedure described for Compound 336 with appropriate variations in reactants, quantities of reagents. LCMS: 526.20 [M+H]⁺. ¹H-NMR (400 MHz, DMSO-d6): δ 8.44 (s, 1H), 8.30 (s, 1H), 7.73 (dd, 1H), 7.64 (d, 1H), 7.58 (dt, 1H), 7.54-7.49 (m, 2H), 7.39 (dd, 1H), 7.05 (s, 1H), 5.08 (s, 2H), 3.71 (s, 2H), 3.16 (s, 3H), 2.99-2.94 m, 1H), 2.60-2.53 (m, 2H), 2.46 (brs, 4H), 2.42-2.36 (m, 1H), 1.74-1.68 (m, 1H), 1.60-1.48 (m, 2H), 1.33-1.23 (m, 2H), 1.08-0.95 (m, 1H).

### Example 29. 2-(6-Chloro-4-((methyl(3,3,3-trifluoropropyl)amino)methyl)-pyridin-2-yl)-6-(3-((4-methyl-4H-1,2,4-triazol-3-yl)methyl)oxetan-3-yl)isoindolin-1-one (Compound 340)

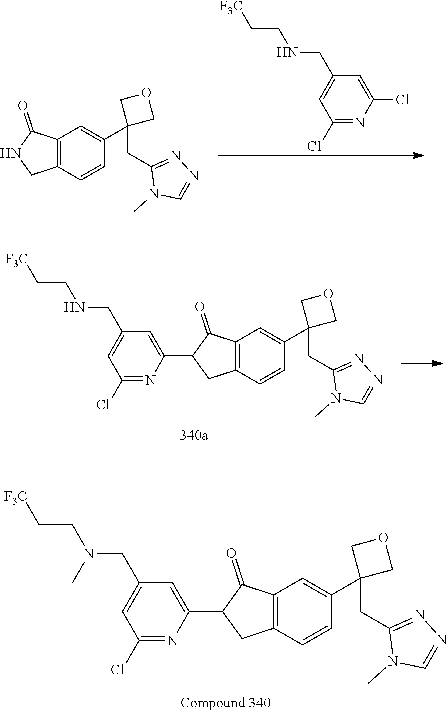 [SMILES: Cn1cnnc1CC1(c2ccc3c(c2)C(=O)NC3)COC1 ; FC(F)(F)CCNCc1cc(Cl)nc(Cl)c1 ; Cn1cnnc1CC1(c2ccc3c(c2)C(=O)C(c2cc(CNCCC(F)(F)F)cc(Cl)n2)C3)COC1 ; CN(CCC(F)(F)F)Cc1cc(Cl)nc(C2Cc3ccc(C4(Cc5nncn5C)COC4)cc3C2=O)c1]

### a) 2-(6-Chloro-4-(((3,3,3-trifluoropropyl)amino)methyl)pyridin-2-yl)-6-(3-((4-methyl-4H-1,2,4-triazol-3-yl)methyl)oxetan-3-yl)isoindolin-1-one (340a)

The compound was prepared according to the procedure described for Compound 128 with appropriate variations in reactants, quantities of reagents. LCMS: 521.1 [M+H]⁺.

### b) 2-(6-Chloro-4-((methyl(3,3,3-trifluoropropyl)amino)methyl)pyridin-2-yl)-6-(3-((4-methyl-4H-1,2,4-triazol-3-yl)methyl)oxetan-3-yl)isoindolin-1-one (Compound 340)

The compound was prepared according to the procedure described for Compound 335 with appropriate variations in reactants, quantities of reagents. LCMS: 535.3 [M+H]⁺. ¹HNMR (400 MHz, DMSO-d6): δ 8.47 (d, 1H), 8.21 (s, 1H), 7.62 (d, 1H), 7.51 (d, 1H), 7.37 (dd, 1H), 7.22 (d, 1H), 5.03 (s, 2H), 4.98 (d, 2H), 4.91 (d, 2H), 3.63 (s, 2H), 3.57 (s, 2H), 3.02 (s, 3H), 2.68-2.63 (m, 2H), 2.58-2.54 (m, 2H), 2.21 (s, 3H).

### ABBREVIATIONS

BOC—tert-Butyloxycarbonyl
DCM—Dichloromethane
DIPAL-H—Diisobutylaluminium hydride
DIPEA—N,N-diisopropylethylamine
DMA—Dimethylacetamide
DMF—N,N-Dimethylformamide
DMSO—Dimethylsulfoxide
EDCl—1-Ethyl-3-[3-dimethylaminopropyl]carbodiimide
HATU—2-(7-Aza-1H-benzotriazole-1-yl)-1,1,3,3-tetramethyluronium hexafluoro-phosphate
HOBT—1-hydroxybenzotriazol
HPLC—High-performance liquid chromatography
LCMS—Liquid chromatography-mass spectrometry
MTBE—Methyl tert-butyl ether
NCS—N-Chlorosuccinimide
Pd(amphos)Cl₂—Bis(di-tert-butyl(4-dimethylaminophenyl)phosphine)dichloro-palladium(II)
Pd(dppf)Cl₂—[1,1′-Bis(diphenylphosphino)ferrocene]palladium(II) dichloride
Pd₂(dba)₃—Tris(dibenzylideneacetone)dipalladium(0)
[Rh(COD)Cl]₂—Cyclooctadiene rhodium chloride dimer
RT—Room temperature
STAB—Sodium triacetoxyborohydride
TFA—Trifluoroacetic acid
THF—Tetrahydrofuran
TLC—Thin layer chromatography
XantPhos—4,5-Bis(diphenylphosphino)-9,9-dimethylxanthene

### EXPERIMENTS

### Experiment 1. Biochemical Assay for CBL-b Inhibition

The inhibitory activity of the test compounds was assessed by Ligand displacement TR-FRET assay. The human recombinant CBL-b (human Biotin CBL-b recombinant protein a.a. 39-426; synthesised in-house) was diluted in enzyme assay buffer (20 mM HEPES, 150 mM NaCl, 0.01% Triton X, 0.01% BSA, 0.5 mM TCEP) in a 10 μl volume and plated in 384-well plates (Cat #781075, Greiner) to a final concentration of 50 nM. The enzyme/compound mixture was incubated at RT for 60 min. The Bodipy FL-Probe described in WO 2020/210508 (synthesized in-house) diluted in enzyme assay buffer was added to the plate and incubated for 60 min RT, the final probe concentration was 0.3 μM. After 60 min incubation, 10 μl of detection mix (Terbium Streptavidin, (Cat #PV3576, Invitrogen) diluted in 1× Lance buffer) was added and incubated for 60 min at room temperature. Time-resolved fluorescence (excitation, 320 nm; emission donor, 615 nm; emission acceptor, 665 nm) was monitored by using 2030 multilabel reader Victor5 (PerkinElmer). The IC₅₀ (concentration for half maximal inhibition) and % maximum inhibition of the compounds were estimated by fitting the dose response data to a sigmoidal curve fitting equation using Graph pad Prism software V. 8.

The compounds of the invention were screened in the above mentioned assay and the IC₅₀ values of the compounds are set forth in Table 1 below wherein “A” refers to a group of compounds having IC₅₀ value of less than 0.08 μM, “B” refers to a group of compounds having IC₅₀ value in range of 0.08 to 0.3 μM and “C” refers to a group of compounds having IC₅₀ value in range of 0.3 μM to 5.0 μM.

TABLE 1

| Group | Compound No. |
|---|---|
| A | 2, 3, 4, 6, 10, 12, 18, 19, 25, 26, 27, 39, 40, 42, 52, 53, 57, |
| | 60, 64, 66, 78, 80, 82, 83, 85, 86, 89, 90, 91, 94, 101, 102, |
| | 103, 104, 105, 107, 108, 109, 110, 111, 112, 113, 116, 118, |
| | 119, 121, 122, 123, 124, 125, 126, 127, 131, 132, 133, 134, |
| | 141, 143, 144, 145, 147, 148, 151, 152, 156, 157, 158, 159, |
| | 160, 161, 162, 163, 164, 166, 167, 168, 169, 170, 171, 172, |
| | 175, 178, 179, 181, 182, 185, 187, 188, 189, 190, 193, 195, |
| | 196, 197, 198, 199, 201, 202, 205, 206, 207, 209, 210, 213, |
| | 214, 215, 216, 218, 219, 221, 222, 223, 224, 225, 227, 230, |
| | 231, 232, 233, 234, 237, 239, 240, 244, 246, 247, 248, 251, |
| | 253, 255, 256, 258, 259, 260, 261, 262, 263, 265, 269, 273, |
| | 274, 275, 277, 278, 279, 280, 281, 282, 289, 293, 294, 295, |
| | 296, 298, 300, 302, 303, 304, 305, 307, 308, 309, 310, 311, |
| | 312, 314, 315, 319, 320, 323, 325, 326, 328, 329 and 337 |
| B | 1, 5, 7, 9, 11, 13, 15, 16, 20, 23, 28, 29, 34, 35, 37, 44, 47, |
| | 48, 49, 56, 59, 62, 63, 65, 67, 68, 73, 74, 84, 87, 106, 117, |
| | 129, 130, 137, 149, 150, 153, 154, 174, 180, 183, 184, 186, |
| | 194, 203, 204, 217, 220, 226, 235, 238, 242, 243, 245, 250, |
| | 252, 254, 264, 267, 271, 272, 276, 283, 286, 287, 291, 292, |
| | 297, 301, 306, 316, 322, 331, 332, 335, 338 and 340 |
| C | 8, 14, 17, 21, 22, 30, 31, 32, 33, 36, 38, 41, 43, 45, 46, 50, |
| | 51, 54, 55, 58, 61, 69, 70, 71, 72, 75, 76, 77, 79, 81, 88, 92, |
| | 96, 97, 98, 99, 100, 114, 128, 136, 138, 139, 140, 142, 165, |
| | 173, 177, 191, 192, 200, 208, 211, 212, 228, 229, 236, 241, |
| | 249, 257, 266, 268, 270, 285, 288, 290, 299, 313, 317, 318, |
| | 324, 327, 333, 334, 336 and 339 |

### Experiment 2. Jurkat NFAT Reporter Assay Protocol

A human T lymphocyte Jurkat reporter cell line was further used to assess activation of the nuclear factor of activated T-cells (NFAT) signaling pathway by Cbl-b inhibitors. The reporter cell line is a genetically engineered Jurkat T cell line that expresses a luciferase reporter driven by an NFAT-response element (NFAT-RE). The Cbl-b inhibitor was incubated with 1×10⁵ Jurkat NFAT reporter cells per well for 30 minutes. Subsequently, cells were stimulated with soluble anti-human CD3/CD28 antibody for 6 hours resulting in NFAT-RE-mediated luminescence. The bioluminescent signal is detected and quantified using Bio-Glo™ Luciferase Assay System. Readouts were reported as fold change over baseline. Here, baseline was the measurement obtained from Jurkat NFAT reporter cells stimulated with anti-CD3 antibody and anti-CD28 antibody, where the cells were not incubated with a Cbl-b inhibitor.

The compounds of the invention were screened in the above mentioned assay and the EC₅₀ values of the compounds are set forth in Table 2 below wherein “A” refers to a group of compounds having EC₅₀ value of less than 0.3 μM, “B” refers to a group of compounds having EC₅₀ value in range of 0.3 to 1.0 μM and “C” refers to a group of compounds having EC₅₀ value in range of 1.0 μM to 10.0 μM.

TABLE 2

| Group | Compound No. |
|---|---|
| A | 1, 2, 4, 6, 10, 26, 27, 39, 40, 52, 56, 57, 60, 64, 66, 78, 80, |
| | 82, 83, 85, 86, 89, 90, 91, 101, 102, 103, 104, 105, 107, |
| | 108, 109, 110, 111, 112, 113, 121, 122, 124, 125, 126, 127, |
| | 131, 132, 134, 143, 144, 145, 148, 151, 152, 157, 159, 162, |
| | 166, 167, 168, 169, 170, 171, 179, 182, 196, 199, 201, 202, |
| | 205, 206, 207, 213, 214, 219, 221, 222, 223, 225, 231, 232, |
| | 233, 239, 240, 244, 246, 247, 248, 251, 253, 260, 262, 265, |
| | 274, 275, 278, 279, 281, 282, 295, 298, 305, 307, 309, 310, |
| | 315, 319, 323 and 329 |
| B | 7, 9, 11, 12, 15, 16, 19, 20, 25, 28, 34, 35, 42, 44, 48, 49, |
| | 53, 59, 62, 65, 67, 68, 84, 87, 88, 93, 94, 106, 119, 123, |
| | 147, 156, 160, 161, 163, 172, 175, 178, 181, 188, 197, 209, |
| | 215, 218, 218, 224, 227, 230, 269, 280, 293, 300, 302, 303, |
| | 314, 320 and 328 |
| C | 3, 5, 8, 13, 14, 17, 18, 23, 29, 30, 31, 33, 37, 45, 46, 47, 51, |
| | 58, 61, 71, 73,74, 92, 116, 117, 133, 158, 164, 185, 187, |
| | 189, 190, 193, 195, 198, 210, 216, 234, 237, 255, 256, 259, |
| | 261, 263, 273, 277, 294, 296, 304, 308, 311, 325, 326 and |
| | 337 |

### Experiment 3. Human PBMC Cytokine Release Assay

Peripheral blood mononuclear cells (PBMC) were isolated from healthy human donor using Histopaque (Sigma). The Cbl-inhibitor was added to 1×10⁵ cells per well and the plate was incubated for 30 minutes at 37° C. in 5% CO₂ at different concentrations with a final DMSO concentration of <0.5%. Following incubation with the Cbl-b inhibitor, human PBMCs were stimulated with Dynabeads™ Human T-Activator CD3/CD28 (Thermo Fisher Scientific) for 48 hours. Cell free supernatant were collected after end of incubation and were analyzed for cytokine secretion, including IL-2 by ELISA (R&D Systems). Readouts were reported as fold change over baseline. Here, baseline was the measurement obtained from human PBMCs stimulated with Dynabeads™ Human T-Activator CD3/CD28, where the cells were not incubated with a Cbl-b inhibitor.

Some compounds of the invention were screened in the above mentioned assay and the EC₅₀ values of the compounds are set forth in Table 3 below wherein “A” refers to a group of compounds having EC₅₀ value of less than 5.0 μM.

| Group | Compound No. |
|---|---|
| A | 1, 2, 4, 6, 11, 12, 25, 35, 39, 40, 52, 53, 56, 57, 59, 78 and 80 |

1. A compound of formula (I) or a pharmaceutically acceptable salt thereof 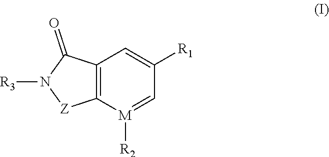
wherein
Z is CH₂ or NH;
M is C or N;
when M is C, then R₂ is hydrogen, halogen, C₁₋₇ alkoxy, cyano, halogen C₁₋₇ alkyl or —NH—C₁₋₇ alkyl;
when M is N, then R₂ is absent;
R₁ is any of the following groups 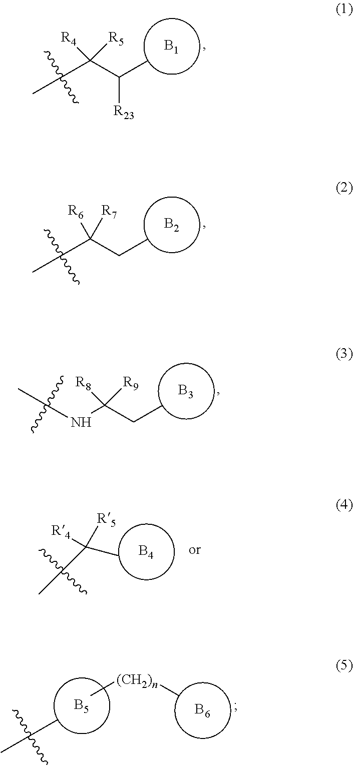
R₄, R₅, R₆, R₇, R₈ and R₉, are, independently, hydrogen, C₁₋₇ alkyl, halogen or halogen C₁₋₇ alkyl, or R₄ and R₅, or R₆ and R₇, or R₈ and R₉ together with the carbon atom to which they are attached form an optionally substituted C₃₋₇ cycloalkyl ring or an optionally substituted 3-6 membered heterocyclic ring having 1-3 heteroatoms as ring atoms independently selected from O, S and N;
R′₄ and R′₅ together with the carbon atom to which they are attached form an optionally substituted C₃-C₆ cycloalkyl ring or an optionally substituted 3-6 membered heterocyclic ring having 1-3 heteroatoms as ring atoms independently selected from O, S and N;
B₁, B₂, B₃, and B₄ are, independently, an optionally substituted 5-6 membered heterocyclic ring having 1-4 heteroatoms as ring atoms independently selected from O, S and N;
B₅ is an optionally substituted phenyl ring or an optionally substituted 5-6 membered heterocyclic ring having 1-4 heteroatoms as ring atoms independently selected from O, S and N;
B₆ is an optionally substituted 5-10 membered monocyclic, bicyclic or spiro bicyclic heterocyclic ring containing 1-4 heteroatoms as ring atoms independently selected from O, S and N;
R₃ is any of the following groups 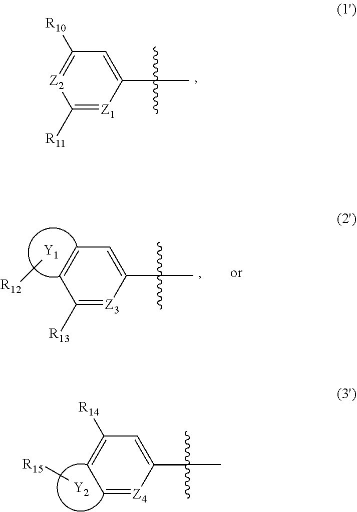
Z₁, Z₂, Z₃ and Z₄ are, independently, CH, CX, C—CF₃ or N;
X is a halogen;
Y₁ and Y₂ are, independently, a 3-6 membered carbocyclic ring or a 5-6 membered heterocyclic ring having 1-4 heteroatoms as ring atoms independently selected from O, S and N;
R₁₁, R₁₃ and R₁₅ are, independently, hydrogen, C₁₋₇ alkyl, C₂₋₇ alkenyl, C₃₋₇ cycloalkyl, C₃₋₇ cycloalkyl C₁₋₇ alkyl, halogen, C₁₋₇ alkoxy, halogen C₁₋₇ alkyl, or a 3-6 membered heterocyclic ring having 1-3 heteroatoms as ring atoms independently selected from O, S and N;
R₁₂ is hydrogen, C₁₋₇ alkyl or —C₁₋₇ alkyl-NR₁₆R₁₇;
R₁₀ and R₁₄, are, independently, —C(O)O—C₁₋₇ alkyl, hydroxy C₁₋₇ alkyl, halogen C₁₋₇ alkyl, —NR₂₁R₂₂, —C₁₋₇ alkyl-NR₂₁R₂₂, -hydroxy C₁₋₇ alkyl-NR₂₁R₂₂, or a group 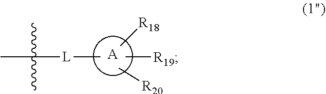
L is a bond, —CH₂—, —CH—, —CH(CH₃)—, —C(CH₂)— or —C(O)—;
A is a 5-10 membered monocyclic, bicyclic or spiro bicyclic heterocyclic ring containing 1-4 heteroatoms as ring atoms independently selected from O, S and N;
R₁₆ and R₁₇ are, independently, hydrogen or C₁₋₇ alkyl;
R₁₈, R₁₉ and R₂₀ are, independently, absent, hydrogen, C₁₋₇ alkyl, C₁₋₇ alkoxy, halogen, cyano, halogen C₁₋₇ alkyl, hydroxy, hydroxy C₁₋₇ alkyl or oxo;
R₂₁ and R₂₂ are, independently, hydrogen, C₁₋₇ alkyl, C₃₋₇ cycloalkyl, C₃₋₇ cycloalkyl C₁₋₇ alkyl, C₁₋₇ alkyl C₃₋₇ cycloalkyl, (C₃₋₇ cycloalkyl)₂ C₁₋₇ alkyl, C₁₋₇ alkyl C₃₋₇ cycloalkyl C₁₋₇ alkyl, C₃₋₇ cycloalkyl halogen C₁₋₇ alkyl, halogen C₁₋₇ alkyl C₃₋₇ cycloalkyl C₁₋₇ alkyl, halogen C₁₋₇ alkyl, halogen C₃₋₇ cycloalkyl, halogen C₃₋₇ cycloalkyl C₁₋₇ alkyl, hydroxy C₁₋₇ alkyl, hydroxy C₁₋₇ alkyl C₃₋₇ cycloalkyl C₁₋₇ alkyl, hydroxy C₃₋₇ cycloalkyl, C₁₋₇ alkoxy C₃₋₇ cycloalkyl, cyano C₃₋₇ cycloalkyl C₁₋₇ alkyl, hydroxy C₃₋₇ cycloalkyl C₁₋₇ alkyl, C₁₋₇ alkoxy C₃₋₇ cycloalkyl C₁₋₇ alkyl, C₁₋₇ alkoxy C₁₋₇ alkyl, aminohydroxy C₁₋₇ alkyl, C₁₋₇ alkyl amide C₁₋₇ alkyl, —SO₂—C₁₋₇ alkyl, —S(O)—C₁₋₇ alkyl, —C₁₋₇ alkyl-(O—C₁₋₇ alkyl)₁₋₃-NH₂, —C₁₋₇ alkyl (C₃₋₇ cycloalkyl)(OH), or a group 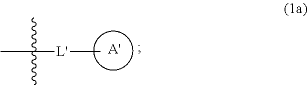
L′ is a bond, C₁₋₇ alkyl or C₂₋₇ alkenyl;
A′ is an optionally substituted 3-6 membered heterocyclic ring having 1-3 heteroatoms as ring atoms independently selected from O, S and N;
R₂₃ is independently, hydrogen, C₁₋₇ alkyl or halogen;
n is 0, 1 or 2;
wherein optional substitution, in each occurrence, is 1-2 substituents independently selected from C₁₋₇ alkyl, C₃₋₇ cycloalkyl, halogen, hydroxy, C₁₋₇ alkoxy, halogen C₁₋₇ alkyl and cyano.

2. A compound according to claim 1, wherein R₁ is group (1) or (5)

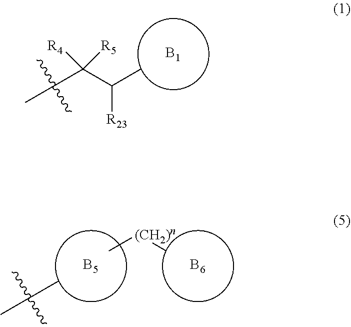

3. A compound according to any one of the preceding claims, wherein Z is CH₂.

4. A compound according to any one of the preceding claims, wherein M is C.

5. A compound according to any one of the preceding claims, wherein R₂ is hydrogen.

6. A compound according to any one of the preceding claims, wherein R₄ and R₅ are, independently, hydrogen or C₁₋₇ alkyl.

7. A compound according to claim 6, wherein R₄ is hydrogen and R₅ is C₁₋₇ alkyl.

8. A compound according to any one of the preceding claims, wherein R₄ and R₅ together with the carbon atom to which they are attached form an optionally substituted C₃-C₆ cycloalkyl ring or an optionally substituted 3-6 membered heterocyclic ring having 1-3 heteroatoms as ring atoms independently selected from O, S and N.

9. A compound according to claim 8, wherein R₄ and R₅ together with the carbon atom to which they are attached form an optionally substituted oxetanyl ring.

10. A compound according to claim 8, wherein R₄ and R₅ together with the carbon atom to which they are attached form an optionally substituted C₃-C₆ cycloalkyl ring.

11. A compound according to claim 10, wherein R₄ and R₅ together with the carbon atom to which they are attached form an optionally substituted cyclobutanyl ring.

12. A compound according to any one of the preceding claims, wherein B₆ is an optionally substituted 5-6 membered heterocyclic ring having 1-4 heteroatoms as ring atoms independently selected from O, S and N;

13. A compound according to any one of the preceding claims, wherein B₁, B₂, B₃, B₄ and B₆ are, independently, a triazolyl ring optionally substituted with one C₁₋₇ alkyl substituent.

14. A compound according to claim 13, wherein C₁₋₇ alkyl substituent is methyl.

15. A compound according to any one of the preceding claims, wherein R₂₃ is hydrogen.

16. A compound according to any one of the preceding claims, wherein B₅ is an optionally substituted phenyl ring.

17. A compound according to any one of the preceding claims, wherein B₆ is a triazolyl ring optionally substituted with one C₁₋₇ alkyl substituent.

18. A compound according to any one of the preceding claims, wherein n is 0.

19. A compound according to any one of the preceding claims, wherein R₃ is any of the following groups:

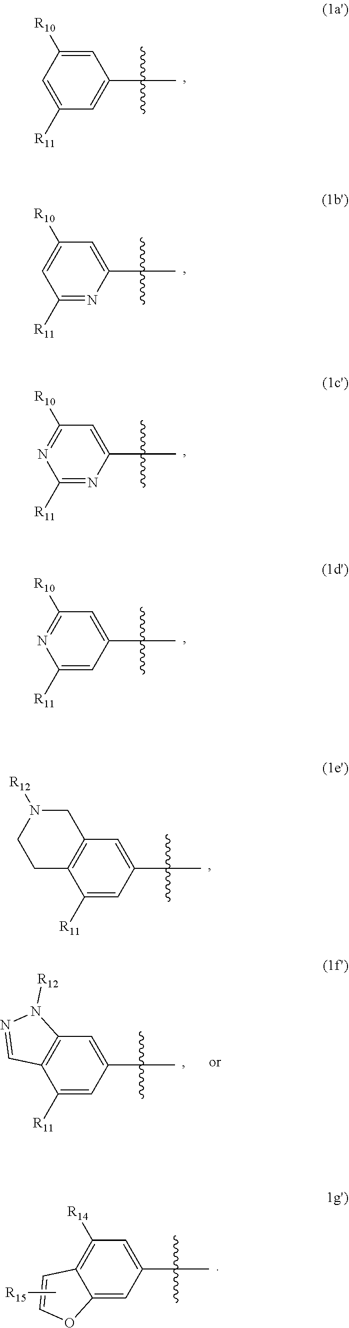

20. A compound according to claim 19, wherein R₃ is group (1b′).

21. A compound according to claim 19 or 20, wherein R₁₀ and R₁₄ is a group 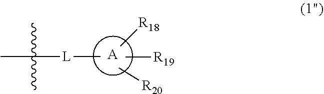
wherein -L- is —CH₂— or —CH(CH₃)—.

22. A compound according to claim 21, wherein A is a 5-10 membered monocyclic ring containing 1-4 heteroatoms as ring atoms independently selected from O, S and N.

23. A compound according to claim 22, wherein A is any of the following groups:

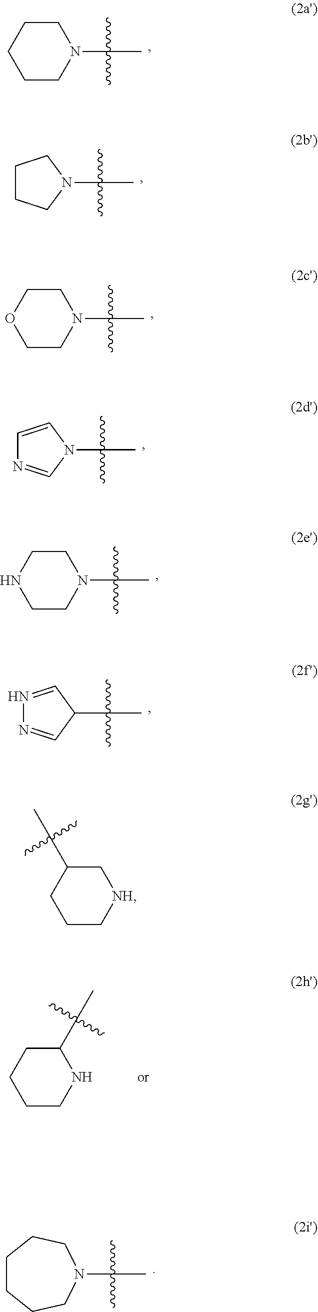

24. A compound according to claim 22, wherein A is group (2a′).

25. A compound according to claim 21, wherein A is a 5-10 membered bicyclic ring or spiro bicyclic heterocyclic ring containing 1-4 heteroatoms as ring atoms independently selected from O, S and N.

26. A compound according to claim 25, wherein A is any of the following groups:

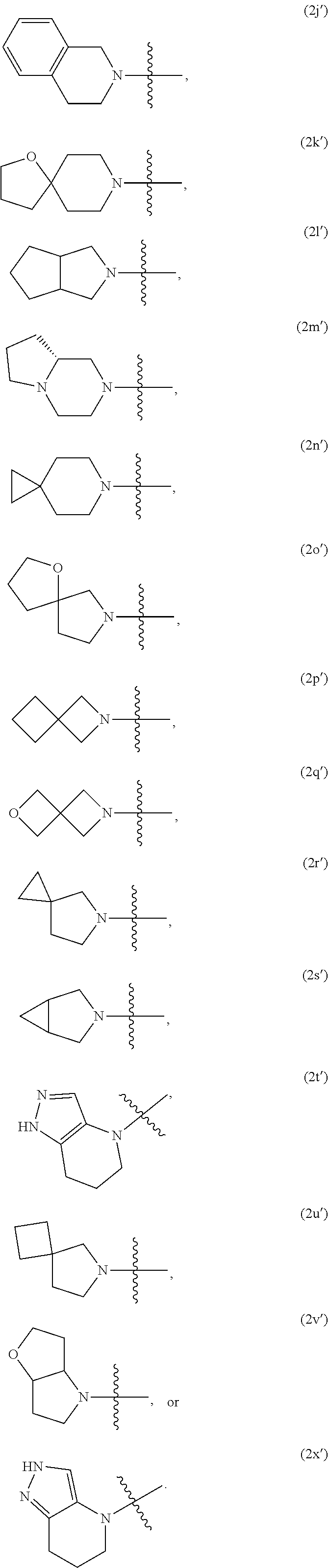

27. A compound according to claim 19 or 20, wherein R₁₀ is —C₁₋₇ alkyl-NR₂₁R₂₂.

28. A compound according to claim 27, wherein R₂₁ is hydrogen.

29. A compound according to claim 27 or 28, wherein R₂₂ is C₃₋₇ cycloalkyl, C₃₋₇ cycloalkyl C₁₋₇ alkyl, halogen C₁₋₇ alkyl, halogen C₃₋₇ cycloalkyl C₁₋₇ alkyl, hydroxy C₁₋₇ alkyl, C₁₋₇ alkyl C₃₋₇ cycloalkyl C₁₋₇ alkyl, C₁₋₇ alkoxy C₃₋₇ cycloalkyl, hydroxy C₃₋₇ cycloalkyl or hydroxy C₃₋₇ cycloalkyl C₁₋₇ alkyl or a group 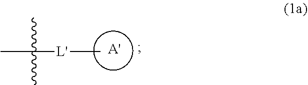
wherein
L′ is a bond or C₁₋₇ alkyl; and
A′ is an optionally substituted 3-6 membered heterocyclic ring having 1-3 heteroatoms as ring atoms independently selected from O, S and N.

30. A compound according to claim 1, having the structure (Ta) 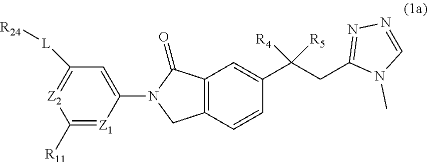
wherein Z₁ and Z₂ are, independently, CH, CX or N;
R₄ and R₅ are, independently hydrogen or C₁₋₇ alkyl, or R₄ and R₅ together with the carbon atom to which they are attached form optionally substituted cyclobutyl ring or an optionally substituted oxetane ring, wherein the optional substitution, in each occurrence, is 1-2 substituents independently selected from C₁₋₇ alkyl, halogen and cyano;
R₁₁ is hydrogen, C₁₋₇ alkyl, C₂₋₇ alkenyl, C₃₋₇ cycloalkyl, C₃₋₇ cycloalkyl C₁₋₇ alkyl, halogen, C₁₋₇ alkoxy or halogen C₁₋₇ alkyl;
L is —CH₂— or —CH(CH₃)—;
R₂₄ is —NR₂₁R₂₂ or a group 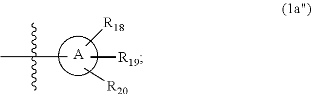
wherein A is a 5-10 membered monocyclic or bicyclic ring or spiro bicyclic heterocyclic ring containing 1-4 heteroatoms as ring atoms independently selected from O, S and N;
R₁₈, R₁₉ and R₂₀ are, independently, hydrogen, C₁₋₇ alkyl, halogen, cyano, halogen C₁₋₇ alkyl, hydroxy, hydroxy C₁₋₇ alkyl or oxo;
R₂₁ and R₂₂ are, independently, hydrogen, C₁₋₇ alkyl, C₃₋₇ cycloalkyl, C₃₋₇ cycloalkyl C₁₋₇ alkyl, C₁₋₇ alkyl C₃₋₇ cycloalkyl, C₁₋₇ alkyl C₃₋₇ cycloalkyl C₁₋₇ alkyl, halogen C₁₋₇ alkyl, halogen C₃₋₇ cycloalkyl or halogen C₃₋₇ cycloalkyl C₁₋₇ alkyl, hydroxy C₁₋₇ alkyl, C₁₋₇ alkoxy C₃₋₇ cycloalkyl, hydroxy C₃₋₇ cycloalkyl C₁₋₇ alkyl or hydroxy C₃₋₇ cycloalkyl.

31. A compound according to claim 30, wherein R₄ and R₅ together with the carbon atom to which they are attached form optionally substituted cyclobutyl ring or an optionally substituted oxetane ring, wherein the optional substitution, in each occurrence, is 1-2 substituents independently selected from C₁₋₇ alkyl, halogen and cyano;

32. A compound according to any one of the preceding claims, wherein R₄ is hydrogen and R₅ is C₁₋₇ alkyl, or R₄ and R₅ together with the carbon atom to which they are attached form a group of formula (3a) or (3b) 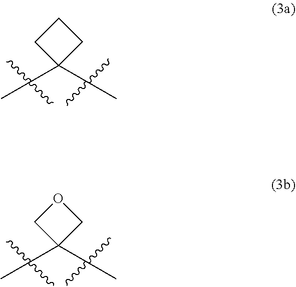 [SMILES: *C1(*)CCC1 ; *C1(*)COC1]
which groups may be optionally substituted with 1-2 substituents independently selected from C₁₋₇ alkyl, halogen and cyano.

33. A compound according to claim 32, wherein R₄ and R₅ together with the carbon atom to which they are attached form a group of formula (3a) or (3b) 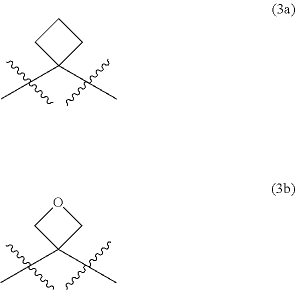 [SMILES: *C1(*)CCC1 ; *C1(*)COC1]
which groups may be optionally substituted with 1-2 substituents independently selected from C₁₋₇ alkyl, halogen and cyano.

34. A compound according to any one of the preceding claims, wherein R₂₁ is hydrogen and R₂₂ is C₃₋₇ cycloalkyl, C₃₋₇ cycloalkyl C₁₋₇ alkyl, halogen C₁₋₇ alkyl or halogen C₃₋₇ cycloalkyl C₁₋₇ alkyl, C₁₋₇ alkoxy C₃₋₇ cycloalkyl or hydroxy C₃₋₇ cycloalkyl.

35. A compound according to claim 1, having the structure (Ib) 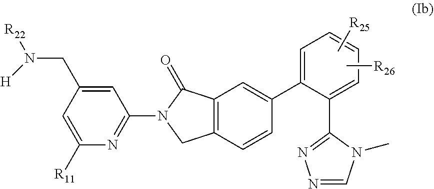
wherein
R₂₅ and R₂₆ are, independently, hydrogen, C₁₋₇ alkyl, C₁₋₇ alkoxy, halogen or cyano;
R₁₁ is hydrogen, C₁₋₇ alkyl or halogen;
R₂₂ is C₁₋₇ alkyl, C₃₋₇ cycloalkyl, C₃₋₇ cycloalkyl C₁₋₇ alkyl, C₁₋₇ alkyl C₃₋₇ cycloalkyl, (C₃₋₇ cycloalkyl)₂ C₁₋₇ alkyl, C₁₋₇ alkyl C₃₋₇ cycloalkyl C₁₋₇ alkyl, C₃₋₇ cycloalkyl halogen C₁₋₇ alkyl, halogen C₁₋₇ alkyl C₃₋₇ cycloalkyl C₁₋₇ alkyl, halogen C₁₋₇ alkyl, halogen C₃₋₇ cycloalkyl, halogen C₃₋₇ cycloalkyl C₁₋₇ alkyl, hydroxy C₁₋₇ alkyl, hydroxy C₁₋₇ alkyl C₃₋₇ cycloalkyl C₁₋₇ alkyl, hydroxy C₃₋₇ cycloalkyl, C₁₋₇ alkoxy C₃₋₇ cycloalkyl, cyano C₃₋₇ cycloalkyl C₁₋₇ alkyl, hydroxy C₃₋₇ cycloalkyl C₁₋₇ alkyl, C₁₋₇ alkoxy C₃₋₇ cycloalkyl C₁₋₇ alkyl, C₁₋₇ alkoxy C₁₋₇ alkyl, aminohydroxy C₁₋₇ alkyl, C₁₋₇ alkyl amide C₁₋₇ alkyl, —SO₂—C₁₋₇ alkyl, —S(O)—C₁₋₇ alkyl, —C₁₋₇ alkyl-(O—C₁₋₇ alkyl)₁₋₃-NH₂, —C₁₋₇ alkyl (C₃₋₇ cycloalkyl)(OH), or a group 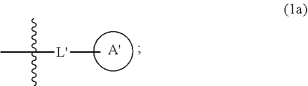
L′ is a bond, C₁₋₇ alkyl or C₂₋₇ alkenyl;
A′ is an optionally substituted 3-6 membered heterocyclic ring having 1-3 heteroatoms as ring atoms independently selected from O, S and N,
wherein optional substitution, in each occurrence, is 1-2 substituents independently selected from C₁₋₇ alkyl, C₃₋₇ cycloalkyl, halogen, hydroxy, C₁₋₇ alkoxy, halogen C₁₋₇ alkyl and cyano.

36. A compound according to claim 35, wherein R₂₂ is C₃₋₇ cycloalkyl, C₃₋₇ cycloalkyl C₁₋₇ alkyl, halogen C₁₋₇ alkyl, halogen C₃₋₇ cycloalkyl, halogen C₃₋₇ cycloalkyl C₁₋₇ alkyl, C₁₋₇ alkoxy C₃₋₇ cycloalkyl, hydroxy C₃₋₇ cycloalkyl or a group 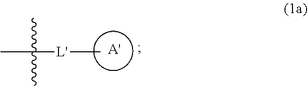
L′ is a bond or C₁₋₇ alkyl;
A′ is an optionally substituted 3-6 membered heterocyclic ring having 1-3 heteroatoms as ring atoms independently selected from O, S and N,
wherein optional substitution, in each occurrence, is 1-2 substituents independently selected from C₁₋₇ alkyl, C₃₋₇ cycloalkyl, halogen, hydroxy, C₁₋₇ alkoxy, halogen C₁₋₇ alkyl and cyano.

37. A compound according to claim 1, wherein the compound is
(S)-6-(3-((4-Methyl-4H-1,2,4-triazol-3-yl)methyl)oxetan-3-yl)-2-(3-((3-methyl-piperidin-1-yl)methyl)-5-(trifluoromethyl)phenyl)isoindolin-1-one (Compound 1);
(S)-6-(3-((4-Methyl-4H-1,2,4-triazol-3-yl)methyl)oxetan-3-yl)-2-(4-((3-methyl-piperidin-1-yl)methyl)-6-(trifluoromethyl)pyridin-2-yl)isoindolin-1-one (Compound 2);
(S)-2-(3-Fluoro-5-((3-methylpiperidin-1-yl)methyl)phenyl)-6-(3-((4-methyl-4H-1,2,4-triazol-3-yl)methyl)oxetan-3-yl)isoindolin-1-one (Compound 3);
(S)-6-(3-((4-Methyl-4H-1,2,4-triazol-3-yl)methyl)oxetan-3-yl)-2-(3-methyl-5-((3-methylpiperidin-1-yl)methyl)phenyl)isoindolin-1-one (Compound 4);
(S)-2-(3-Methoxy-5-((3-methylpiperidin-1-yl)methyl)phenyl)-6-(3-((4-methyl-4H-1,2,4-triazol-3-yl)methyl)oxetan-3-yl)isoindolin-1-one (Compound 5);
(S)-2-(3-Chloro-5-((3-methylpiperidin-1-yl)methyl)phenyl)-6-(3-((4-methyl-4H-1,2,4-triazol-3-yl)methyl)oxetan-3-yl)isoindolin-1-one (Compound 6);
(S)-2-(3-Cyclopropyl-5-((3-methylpiperidin-1-yl)methyl)phenyl)-6-(3-((4-methyl-4H-1,2,4-triazol-3-yl)methyl)oxetan-3-yl)isoindolin-1-one (Compound 7);
(S)-6-(3-((4-Methyl-4H-1,2,4-triazol-3-yl)methyl)oxetan-3-yl)-2-(3-((3-methyl-piperidin-1-yl)methyl)phenyl)isoindolin-1-one (Compound 8);
(S)-6-(3-((4-Methyl-4H-1,2,4-triazol-3-yl)methyl)oxetan-3-yl)-2-(3-((3-methyl-piperidin-1-yl)methyl)-5-vinylphenyl)isoindolin-1-one (Compound 9);
(S)-2-(3-Bromo-5-((3-methylpiperidin-1-yl)methyl)phenyl)-6-(3-((4-methyl-4H-1,2,4-triazol-3-yl)methyl)oxetan-3-yl)isoindolin-1-one (Compound 10);
(S)-2-(3-Ethyl-5-((3-methylpiperidin-1-yl)methyl)phenyl)-6-(3-((4-methyl-4H-1,2,4-triazol-3-yl)methyl)oxetan-3-yl)isoindolin-1-one (Compound 11);
6-(3-((4-Methyl-4H-1,2,4-triazol-3-yl)methyl)oxetan-3-yl)-2-(3-methyl-5-(1-((S)-3-methylpiperidin-1-yl)ethyl)phenyl)isoindolin-1-one (Compound 12);
2-(3-(((Cyclopropylmethyl)amino)methyl)-5-(trifluoromethyl)phenyl)-6-(3-((4-methyl-4H-1,2,4-triazol-3-yl)methyl)oxetan-3-yl)isoindolin-1-one (Compound 13);
(S)-6-(3-((4-Methyl-4H-1,2,4-triazol-3-yl)methyl)oxetan-3-yl)-2-(3-((3-methyl-piperidin-1-yl)methyl)-5-(piperidin-1-yl)phenyl)isoindolin-1-one (Compound 14);
(S)-2-(3-Isopropyl-5-((3-methylpiperidin-1-yl)methyl)phenyl)-6-(3-((4-methyl-4H-1,2,4-triazol-3-yl)methyl)oxetan-3-yl)isoindolin-1-one (Compound 15);
(S)-2-(3-(tert-Butyl)-5-((3-methylpiperidin-1-yl)methyl)phenyl)-6-(3-((4-methyl-4H-1,2,4-triazol-3-yl)methyl)oxetan-3-yl)isoindolin-1-one (Compound 16);
(S)-6-(3-((4-Methyl-4H-1,2,4-triazol-3-yl)methyl)oxetan-3-yl)-2-(2-((3-methyl-piperidin-1-yl)methyl)-6-(trifluoromethyl)pyridin-4-yl)isoindolin-1-one (Compound 17);
2-(3-(((Cyclopropylmethyl)amino)methyl)-5-methylphenyl)-6-(3-((4-methyl-4H-1,2,4-triazol-3-yl)methyl)oxetan-3-yl)isoindolin-1-one (Compound 18);
6-(3-((4-Methyl-4H-1,2,4-triazol-3-yl)methyl)oxetan-3-yl)-2-(3-methyl-5-(((3,3,3-trifluoropropyl)amino)methyl)phenyl)isoindolin-1-one (Compound 19);
2-(3-((((2,2-Difluorocyclopropyl)methyl)amino)methyl)-5-methylphenyl)-6-(3-((4-methyl-4H-1,2,4-triazol-3-yl)methyl)oxetan-3-yl)isoindolin-1-one (Compound 20);
(S)-6-(3-((4-Methyl-4H-1,2,4-triazol-3-yl)methyl)oxetan-3-yl)-2-(3-((3-methyl-piperidin-1-yl)methyl)-5-(pyrrolidin-1-yl)phenyl)isoindolin-1-one (Compound 21);
6-(3-((4-Methyl-4H-1,2,4-triazol-3-yl)methyl)oxetan-3-yl)-2-(3-methyl-5-(((2,2,2-trifluoroethyl)amino)methyl)phenyl)isoindolin-1-one (Compound 22);
(S)-6-(3-((4-Methyl-4H-1,2,4-triazol-3-yl)methyl)oxetan-3-yl)-2-(2-methyl-6-((3-methylpiperidin-1-yl)methyl)pyridin-4-yl)isoindolin-1-one (Compound 23);
(S)-2-(3-(Azetidin-1-yl)-5-((3-methylpiperidin-1-yl)methyl)phenyl)-6-(3-((4-methyl-4H-1,2,4-triazol-3-yl)methyl)oxetan-3-yl)isoindolin-1-one (Compound 24);
6-(3-((4-Methyl-4H-1,2,4-triazol-3-yl)methyl)oxetan-3-yl)-2-(6-(trifluoro-methyl)-4-(((3,3,3-trifluoropropyl)amino)methyl)pyridin-2-yl)isoindolin-1-one (Compound 25);
(S)-2-(6-Methyl-4-((3-methylpiperidin-1-yl)methyl)pyridin-2-yl)-6-(3-((4-methyl-4H-1,2,4-triazol-3-yl)methyl)oxetan-3-yl)isoindolin-1-one (Compound 26);
(S)-6-(3-((4-Methyl-4H-1,2,4-triazol-3-yl)methyl)oxetan-3-yl)-2-(6-(trifluoro-methyl)-4-((3-(trifluoromethyl)piperidin-1-yl)methyl)pyridin-2-yl)isoindolin-1-one (Compound 27);
2-(4-((4,4-Difluoro-3-methylpiperidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 28);
1-((2-(6-(3-((4-Methyl-4H-1,2,4-triazol-3-yl)methyl)oxetan-3-yl)-1-oxoisoindo-lin-2-yl)-6-(trifluoromethyl)pyridin-4-yl)methyl)piperidine-3-carbonitrile (Compound 29);
(S)-2-(4-((3-Hydroxypiperidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 30);
2-(4-((3,4-Dihydroisoquinolin-2(1H)-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 31);
2-(4-((1-Oxa-8-azaspiro[4.5]decan-8-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 32);
2-(4-((3-Fluoropiperidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 33);
(S)-6-(3-((4-Methyl-4H-1,2,4-triazol-3-yl)methyl)oxetan-3-yl)-2-(4-((3-methyl-pyrrolidin-1-yl)methyl)-6-(trifluoromethyl)pyridin-2-yl)isoindolin-1-one (Compound 34);
2-(4-((Hexahydrocyclopenta[c]pyrrol-2(1H)-yl)methyl)-6-(trifluoromethyl)pyri-din-2-yl)-6-(3-((4-methyl-4H-1,2,4-triazol-3-yl)methyl)oxetan-3-yl)isoindolin-1-one (Compound 35);
(S)-2-(4-(3-Hydroxypyrrolidine-1-carbonyl)-6-(trifluoromethyl)pyridin-2-yl)-6-(3-((4-methyl-4H-1,2,4-triazol-3-yl)methyl)oxetan-3-yl)isoindolin-1-one (Compound 36);
2-(4-((3-(Hydroxymethyl)piperidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 37);
6-(3-((4-Methyl-4H-1,2,4-triazol-3-yl)methyl)oxetan-3-yl)-2-(6-(trifluoro-methyl)-4-((4-(trifluoromethyl)piperidin-1-yl)methyl)pyridin-2-yl)isoindolin-1-one (Compound 38);
2-(4-(((3S,5R)-3,5-Dimethylpiperidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 39);
(S)-2-(6-Chloro-4-((3-methylpiperidin-1-yl)methyl)pyridin-2-yl)-6-(3-((4-methyl-4H-1,2,4-triazol-3-yl)methyl)oxetan-3-yl)isoindolin-1-one (Compound 40);
(R)-2-(4-((Hexahydropyrrolo[1,2-a]pyrazin-2(1H)-yl)methyl)-6-(trifluoro-methyl)pyridin-2-yl)-6-(3-((4-methyl-4H-1,2,4-triazol-3-yl)methyl)oxetan-3-yl)iso-indolin-1-one (Compound 41);
2-(4-(((3S,5S)-3,5-Dimethylpiperidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 42);
2-(4-((4-Hydroxypiperidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 43);
2-(4-((6-Azaspiro[2.5]octan-6-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 44);
6-(3-((4-Methyl-4H-1,2,4-triazol-3-yl)methyl)oxetan-3-yl)-2-(4-((2-methyl-piperidin-1-yl)methyl)-6-(trifluoromethyl)pyridin-2-yl)isoindolin-1-one (Compound 45);
(R)-6-(3-((4-Methyl-4H-1,2,4-triazol-3-yl)methyl)oxetan-3-yl)-2-(4-((2-methyl-morpholino)methyl)-6-(trifluoromethyl)pyridin-2-yl)isoindolin-1-one (Compound 46);
2-(4-((3,3-Difluoropiperidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 47);
2-(4-((4,4-Difluoropiperidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 48);
(S)-2-(3-(Cyclopropylmethyl)-5-((3-methylpiperidin-1-yl)methyl)phenyl)-6-(3-((4-methyl-4H-1,2,4-triazol-3-yl)methyl)oxetan-3-yl)isoindolin-1-one (Compound 49);
2-(4-((1-Oxa-7-azaspiro[4.4]nonan-7-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 50);
2-(4-((2-Azaspiro[3.3]heptan-2-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 51);
2-(6-Chloro-4-(((3S,5R)-3,5-dimethylpiperidin-1-yl)methyl)pyridin-2-yl)-6-(3-((4-methyl-4H-1,2,4-triazol-3-yl)methyl)oxetan-3-yl)isoindolin-1-one (Compound 52);
(S)-6-(3-((4-Methyl-4H-1,2,4-triazol-3-yl)methyl)oxetan-3-yl)-2-(2-methyl-6-((3-methylpiperidin-1-yl)methyl)pyrimidin-4-yl)isoindolin-1-one (Compound 53);
2-(4-((2-Methyl-1H-imidazol-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 54);
2-(4-((2-Oxa-6-azaspiro[3.3]heptan-6-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 55);
2-(3-(((3S,5R)-3,5-Dimethylpiperidin-1-yl)methyl)-5-methylphenyl)-6-(3-((4-methyl-4H-1,2,4-triazol-3-yl)methyl)oxetan-3-yl)isoindolin-1-one (Compound 56);
2-(6-Chloro-4-(((cyclopropylmethyl)amino)methyl)pyridin-2-yl)-6-(3-((4-methyl-4H-1,2,4-triazol-3-yl)methyl)oxetan-3-yl)isoindolin-1-one (Compound 57);
2-(6-Chloro-4-(((S)-3-methylpiperidin-1-yl)methyl)pyridin-2-yl)-6-((1s,3R)-3-methyl-1-(4-methyl-4H-1,2,4-triazol-3-yl)cyclobutyl)isoindolin-1-one (Compound 58);
2-(3-(((3S,5S)-3,5-Dimethylpiperidin-1-yl)methyl)-5-methylphenyl)-6-(3-((4-methyl-4H-1,2,4-triazol-3-yl)methyl)oxetan-3-yl)isoindolin-1-one (Compound 59);
2-(6-Chloro-4-(((3S,5S)-3,5-dimethylpiperidin-1-yl)methyl)pyridin-2-yl)-6-(3-((4-methyl-4H-1,2,4-triazol-3-yl)methyl)oxetan-3-yl)isoindolin-1-one (Compound 60);
(S)-2-(4-((3-Fluoropyrrolidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 61);
2-(4-((5-Azaspiro[2.4]heptan-5-yl)methyl)-6-chloropyridin-2-yl)-6-(3-((4-methyl-4H-1,2,4-triazol-3-yl)methyl)oxetan-3-yl)isoindolin-1-one (Compound 62);
2-(4-((4-Methyl-3-oxopiperazin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 63);
2-(6-Chloro-4-(piperidin-1-ylmethyl)pyridin-2-yl)-6-(3-((4-methyl-4H-1,2,4-triazol-3-yl)methyl)oxetan-3-yl)isoindolin-1-one (Compound 64);
2-(3-(((3S,5R)-3,5-Dimethylpiperidin-1-yl)methyl)-5-(trifluoromethyl)phenyl)-6-(3-((4-methyl-4H-1,2,4-triazol-3-yl)methyl)oxetan-3-yl)isoindolin-1-one (Compound 65);
2-(6-Chloro-4-(((3,3,3-trifluoropropyl)amino)methyl)pyridin-2-yl)-6-(3-((4-methyl-4H-1,2,4-triazol-3-yl)methyl)oxetan-3-yl)isoindolin-1-one (Compound 66);
(S)-2-(6-Chloro-4-((3-methylpyrrolidin-1-yl)methyl)pyridin-2-yl)-6-(3-((4-methyl-4H-1,2,4-triazol-3-yl)methyl)oxetan-3-yl)isoindolin-1-one (Compound 67);
(R)-2-(6-Chloro-4-((3-methylpyrrolidin-1-yl)methyl)pyridin-2-yl)-6-(3-((4-methyl-4H-1,2,4-triazol-3-yl)methyl)oxetan-3-yl)isoindolin-1-one (Compound 68);
2-(4-((5-Methyl-1H-imidazol-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 69);
2-(4-((4-Methyl-1H-imidazol-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 70);
2-(4-((3-Azabicyclo[3.1.0]hexan-3-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 71);
2-(4-(((2R,6S)-2,6-Dimethylmorpholino)methyl)-6-(trifluoromethyl)pyridin-2-yl)-6-(3-((4-methyl-4H-1,2,4-triazol-3-yl)methyl)oxetan-3-yl)isoindolin-1-one (Compound 72);
(S)-2-(6-Cyclopropyl-4-((3-methylpiperidin-1-yl)methyl)pyridin-2-yl)-6-(3-((4-methyl-4H-1,2,4-triazol-3-yl)methyl)oxetan-3-yl)isoindolin-1-one (Compound 73);
2-(4-(((3,3-Difluorocyclobutyl)amino)methyl)-6-(trifluoromethyl)pyridin-2-yl)-6-(3-((4-methyl-4H-1,2,4-triazol-3-yl)methyl)oxetan-3-yl)isoindolin-1-one (Compound 74);
2-(3-(2-(Ethylamino)ethyl)-5-(trifluoromethyl)phenyl)-6-(3-((4-methyl-4H-1,2,4-triazol-3-yl)methyl)oxetan-3-yl)isoindolin-1-one (Compound 75);
2-(1-(2-(Dimethylamino)ethyl)-4-methyl-1H-indazol-6-yl)-6-(3-((4-methyl-4H-1,2,4-triazol-3-yl)methyl)oxetan-3-yl)isoindolin-1-one (Compound 76);
2-(2-Ethyl-5-(trifluoromethyl)-1,2,3,4-tetrahydroisoquinolin-7-yl)-6-(3-((4-methyl-4H-1,2,4-triazol-3-yl)methyl)oxetan-3-yl)isoindolin-1-one (Compound 77);
2-(4-(((3S,5R)-3,5-Dimethylpiperidin-1-yl)methyl)-6-methylpyridin-2-yl)-6-(3-((4-methyl-4H-1,2,4-triazol-3-yl)methyl)oxetan-3-yl)isoindolin-1-one (Compound 78);
2-(6-Chloro-4-((diethylamino)methyl)pyridin-2-yl)-6-(3-((4-methyl-4H-1,2,4-triazol-3-yl)methyl)oxetan-3-yl)isoindolin-1-one (Compound 79);
2-(4-(((Cyclopropylmethyl)amino)methyl)-6-(trifluoromethyl)pyridin-2-yl)-6-(3-((4-methyl-4H-1,2,4-triazol-3-yl)methyl)oxetan-3-yl)isoindolin-1-one (Compound 80);
2-(4-((5-Methyl-2,5-diazabicyclo[2.2.1]heptan-2-yl)methyl)-6-(trifluoromethyl)-pyridin-2-yl)-6-(3-((4-methyl-4H-1,2,4-triazol-3-yl)methyl)oxetan-3-yl)isoindolin-1-one (Compound 81);
2-(4-(((3S,5R)-3,5-Dimethylpiperidin-1-yl)methyl)-6-(trifluoromethyl)pyridin-2-yl)-6-(1-((4-methyl-4H-1,2,4-triazol-3-yl)methyl)cyclobutyl)isoindolin-1-one (Compound 82);
2-(6-Chloro-4-(((S)-3-methylpiperidin-1-yl)methyl)pyridin-2-yl)-6-(1-(4-methyl-4H-1,2,4-triazol-3-yl)propan-2-yl)isoindolin-1-one (Compound 83);
2-(6-Chloro-4-(((3S,5R)-3,5-dimethylpiperidin-1-yl)methyl)pyridin-2-yl)-6-(1-(4-methyl-4H-1,2,4-triazol-3-yl)propan-2-yl)isoindolin-1-one (Compound 84);
2-(6-Chloro-4-(((3S,5R)-3,5-dimethylpiperidin-1-yl)methyl)pyridin-2-yl)-6-(1-(4-methyl-4H-1,2,4-triazol-3-yl)propan-2-yl)isoindolin-1-one (Compound 85);
2-(4-(((3S,5R)-3,5-Dimethylpiperidin-1-yl)methyl)-6-(trifluoromethyl)pyridin-2-yl)-6-((S)-1-(4-methyl-4H-1,2,4-triazol-3-yl)propan-2-yl)isoindolin-1-one (Compound 86);
2-(4-(((3S,5R)-3,5-Dimethylpiperidin-1-yl)methyl)-6-(trifluoromethyl)pyridin-2-yl)-6-((R)-1-(4-methyl-4H-1,2,4-triazol-3-yl)propan-2-yl)isoindolin-1-one (Compound 87);
6-(3-((4-Methyl-4H-1,2,4-triazol-3-yl)methyl)oxetan-3-yl)-2-(4-(((1-methyl-cyclobutyl)amino)methyl)-6-(trifluoromethyl)pyridin-2-yl)isoindolin-1-one (Compound 88);
(S)-2-(6-Chloro-4-((3-methylpiperidin-1-yl)methyl)pyridin-2-yl)-6-(1-((4-methyl-4H-1,2,4-triazol-3-yl)methyl)cyclobutyl)isoindolin-1-one (Compound 89);
2-(6-Chloro-4-(((S)-3-methylpiperidin-1-yl)methyl)pyridin-2-yl)-6-((R)-1-(4-methyl-4H-1,2,4-triazol-3-yl)propan-2-yl)isoindolin-1-one (Compound 90);
2-(6-Chloro-4-(((S)-3-methylpiperidin-1-yl)methyl)pyridin-2-yl)-6-((S)-1-(4-methyl-4H-1,2,4-triazol-3-yl)propan-2-yl)isoindolin-1-one (Compound 91);
2-(4-((1-Methyl-1,5,6,7-tetrahydro-4H-pyrazolo[4,3-b]pyridin-4-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 92);
2-(4-((2,5-Dimethylpiperidin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 93);
(S)-6-(3-((4-Methyl-4H-1,2,4-triazol-3-yl)methyl)oxetan-3-yl)-2-(4-((3-methyl-piperidin-1-yl)methyl)benzofuran-6-yl)isoindolin-1-one (Compound 94);
2-(4-(1H-Pyrazol-4-yl)-6-(trifluoromethyl)pyridin-2-yl)-6-(3-((4-methyl-4H-1,2,4-triazol-3-yl)methyl)oxetan-3-yl)isoindolin-1-one (Compound 95);
(R)-6-(3-((4-Methyl-4H-1,2,4-triazol-3-yl)methyl)oxetan-3-yl)-2-(4-((3-methyl-piperazin-1-yl)methyl)-6-(trifluoromethyl)pyridin-2-yl)isoindolin-1-one (Compound 96);
(R)-2-(4-((3,4-Dimethylpiperazin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 97);
2-(4-(((3R,5S)-3,5-Dimethylpiperazin-1-yl)methyl)-6-(trifluoromethyl)pyridin-2-yl)-6-(3-((4-methyl-4H-1,2,4-triazol-3-yl)methyl)oxetan-3-yl)isoindolin-1-one (Compound 98);
2-(4-(13-Amino-5,8,11-trioxa-2-azatridecyl)-6-chloropyridin-2-yl)-6-(3-((4-methyl-4H-1,2,4-triazol-3-yl) methyl)oxetan-3-yl)isoindolin-1-one (Compound 99);
2-(3-(1-Hydroxy-2-(isopropylamino) ethyl)-5-(trifluoromethyl)phenyl)-6-(3-((4-methyl-4H-1,2,4-triazol-3-yl)methyl)oxetan-3-yl)isoindolin-1-one (Compound 100);
6-(4-Chloro-2-(4-methyl-4H-1,2,4-triazol-3-yl)phenyl)-2-(4-(((cyclopropyl-methyl)amino)methyl)-6-methylpyridin-2-yl)isoindolin-1-one (Compound 101);
6-(4-Chloro-2-(4-methyl-4H-1,2,4-triazol-3-yl)phenyl)-2-(6-chloro-4-(((cyclopropylmethyl)amino)methyl)pyridin-2-yl)isoindolin-1-one (Compound 102);
6-(4-Chloro-2-(4-methyl-4H-1,2,4-triazol-3-yl) phenyl)-2-(4-(1-((cyclopropylmethyl)amino)ethyl)-6-methylpyridin-2-yl)isoindolin-1-one (Compound 103);
6-(4-Chloro-2-(4-methyl-4H-1,2,4-triazol-3-yl)phenyl)-2-(6-chloro-4-(1-(cyclopentylamino)ethyl)pyridin-2-yl)isoindolin-1-one, enantiomer 1 (Compound 104);
6-(4-Chloro-2-(4-methyl-4H-1,2,4-triazol-3-yl)phenyl)-2-(6-chloro-4-(1-(cyclopentylamino)ethyl)pyridin-2-yl)isoindolin-1-one, enantiomer 2 (Compound 105);
6-(4-Chloro-2-(4-methyl-4H-1,2,4-triazol-3-yl)phenyl)-2-(6-chloro-4-((cyclopentylamino)methyl)pyridin-2-yl)isoindolin-1-one (Compound 106);
6-(4-Chloro-2-(4-methyl-4H-1,2,4-triazol-3-yl)phenyl)-2-(6-chloro-4-(1-((cyclobutylmethyl)amino)ethyl)pyridin-2-yl)isoindolin-1-one (Compound 107);
6-(4-Chloro-2-(4-methyl-4H-1,2,4-triazol-3-yl)phenyl)-2-(6-chloro-4-(((1-cyclopropylethyl)amino)methyl)pyridin-2-yl)isoindolin-1-one [enantiomer 1](Compound 108);
6-(4-Chloro-2-(4-methyl-4H-1,2,4-triazol-3-yl)phenyl)-2-(6-chloro-4-(((1-cyclopropylethyl)amino)methyl)pyridin-2-yl)isoindolin-1-one [enantiomer 2](Compound 109);
6-(4-Chloro-2-(4-methyl-4H-1,2,4-triazol-3-yl)phenyl)-2-(6-chloro-4-(1-((cyclopropylmethyl)amino)ethyl)pyridin-2-yl)isoindolin-1-one [enantiomer 1](Compound 110);
6-(4-Chloro-2-(4-methyl-4H-1,2,4-triazol-3-yl)phenyl)-2-(6-chloro-4-(1-((cyclopropylmethyl)amino)ethyl)pyridin-2-yl)isoindolin-1-one [enantiomer 2](Compound 111);
(S)-4-(2-(6-Chloro-4-((3-methylpiperidin-1-yl)methyl)pyridin-2-yl)-3-oxoisoindolin-5-yl)-3-(4-methyl-4H-1,2,4-triazol-3-yl)benzonitrile (Compound 112);
(S)-6-(4-Chloro-2-(4-methyl-4H-1,2,4-triazol-3-yl)phenyl)-2-(6-chloro-4-((3-methylpiperidin-1-yl)methyl)pyridin-2-yl)isoindolin-1-one (Compound 113);
(S)-2-(6-Chloro-4-((3-methylpiperidin-1-yl)methyl)pyridin-2-yl)-6-(1-methyl-4-(4-methyl-4H-1,2,4-triazol-3-yl)-1H-pyrazol-5-yl)isoindolin-1-one (Compound 114);
(S)-2-(6-Chloro-4-((3-methylpiperidin-1-yl)methyl)pyridin-2-yl)-6-(2-((4-methyl-4H-1,2,4-triazol-3-yl)methyl)phenyl)isoindolin-1-one (Compound 115);
2-(6-Chloro-4-(((3,3,3-trifluoropropyl)amino)methyl)pyridin-2-yl)-6-(2-(4-methyl-4H-1,2,4-triazol-3-yl)phenyl)isoindolin-1-one (Compound 116);
2-(4-(((3S,5R)-3,5-Dimethylpiperidin-1-yl)methyl)-6-(trifluoromethyl)pyridin-2-yl)-6-(2-(4-methyl-4H-1,2,4-triazol-3-yl)phenyl)isoindolin-1-one (Compound 117);
(S)-2-(6-Chloro-4-((3-methylpiperidin-1-yl)methyl)pyridin-2-yl)-6-(2-(4-methyl-4H-1,2,4-triazol-3-yl)phenyl)isoindolin-1-one (Compound 118);
6-(4-Chloro-2-(4-methyl-4H-1,2,4-triazol-3-yl)phenyl)-2-(3-chloro-5-(((cyclopropylmethyl)amino)methyl)phenyl)isoindolin-1-one (Compound 119);
2-(4-(((Cyclopropylmethyl)amino)methyl)-6-methylpyridin-2-yl)-6-(4-methyl-3-(4-methyl-4H-1,2,4-triazol-3-yl)phenyl)isoindolin-1-one (Compound 120);
6-(4-Chloro-2-(4-methyl-4H-1,2,4-triazol-3-yl)phenyl)-2-(6-chloro-4-(1-(((3,3-difluorocyclobutyl)methyl)amino)ethyl)pyridin-2-yl)isoindolin-1-one (Compound 121);
6-(4-Chloro-2-(4-methyl-4H-1,2,4-triazol-3-yl)phenyl)-2-(4-(((1-cyclopropyl-ethyl)amino)methyl)-6-methylpyridin-2-yl)isoindolin-1-one (Compound 122);
6-(4-Chloro-2-(4-methyl-4H-1,2,4-triazol-3-yl)phenyl)-2-(6-chloro-4-((((tetra-hydrofuran-2-yl)methyl)amino)methyl)pyridin-2-yl)isoindolin-1-one [enantiomer 1](Compound 123);
6-(4-Chloro-2-(4-methyl-4H-1,2,4-triazol-3-yl)phenyl)-2-(6-chloro-4-((((tetra-hydrofuran-2-yl)methyl)amino)methyl)pyridin-2-yl)isoindolin-1-one [enantiomer 2](Compound 124);
6-(4-Chloro-2-(4-methyl-4H-1,2,4-triazol-3-yl)phenyl)-2-(4-(1-(cyclopentyl-amino)ethyl)-6-methylpyridin-2-yl)isoindolin-1-one [enantiomer 1](Compound 125);
6-(4-Chloro-2-(4-methyl-4H-1,2,4-triazol-3-yl)phenyl)-2-(4-(1-(cyclopentyl-amino)ethyl)-6-methylpyridin-2-yl)isoindolin-1-one [enantiomer 2](Compound 126);
2-(4-(((Cyclopropylmethyl)amino)methyl)-6-methylpyridin-2-yl)-6-(4-fluoro-2-(4-methyl-4H-1,2,4-triazol-3-yl)phenyl)isoindolin-1-one (Compound 127);
2-(6-Chloro-4-(((1-cyclopropylethyl)amino)methyl)pyridin-2-yl)-6-(3-((4-methyl-4H-1,2,4-triazol-3-yl)methyl)oxetan-3-yl)isoindolin-1-one (Compound 131);
6-(4-Chloro-2-(4-methyl-4H-1,2,4-triazol-3-yl)phenyl)-2-(4-(((cyclopropyl-methyl)amino)methyl)-6-(trifluoromethyl)pyridin-2-yl)isoindolin-1-one (Compound 132);
2-(6-(((Cyclopropylmethyl)amino)methyl)-2-methylpyrimidin-4-yl)-6-(4-fluoro-2-(4-methyl-4H-1,2,4-triazol-3-yl)phenyl)isoindolin-1-one (Compound 133);
2-(4-(((Cyclobutylmethyl)amino)methyl)-6-methylpyridin-2-yl)-6-(4-fluoro-2-(4-methyl-4H-1,2,4-triazol-3-yl)phenyl)isoindolin-1-one (Compound 134);
2-(6-Chloro-4-(((cyclobutylmethyl)amino)methyl)pyridin-2-yl)-6-(3-((4-methyl-4H-1,2,4-triazol-3-yl)methyl)oxetan-3-yl)isoindolin-1-one (Compound 144);
2-(6-Chloro-4-(((cyclopropylmethyl)amino)methyl)pyridin-2-yl)-6-(1-((4-methyl-4H-1,2,4-triazol-3-yl)methyl)cyclobutyl)isoindolin-1-one (Compound 145);
2-(4-(((Cyclopropylmethyl)amino)methyl)-6-methylpyridin-2-yl)-6-(3-((4-methyl-4H-1,2,4-triazol-3-yl)methyl)oxetan-3-yl)isoindolin-1-one (Compound 147);
2-(6-Chloro-4-((3-ethylpiperidin-1-yl)methyl)pyridin-2-yl)-6-(3-((4-methyl-4H-1,2,4-triazol-3-yl)methyl)oxetan-3-yl)isoindolin-1-one (Compound 148);
2-(6-Chloro-4-(((3,3,3-trifluoropropyl)amino)methyl)pyridin-2-yl)-6-(1-((4-methyl-4H-1,2,4-triazol-3-yl)methyl)cyclobutyl)isoindolin-1-one (Compound 151);
(S)-2-(6-Chloro-4-((3-methylpiperidin-1-yl)methyl)pyridin-2-yl)-6-(3,3-difluoro-1-((4-methyl-4H-1,2,4-triazol-3-yl)methyl)cyclobutyl)isoindolin-1-one (Compound 152);
3-(2-(6-Chloro-4-(((3,3,3-trifluoropropyl)amino)methyl)pyridin-2-yl)-3-oxoisoindolin-5-yl)-3-((4-methyl-4H-1,2,4-triazol-3-yl)methyl)cyclobutane-1-carbonitrile (Compound 158);
2-(6-Chloro-4-(((cyclopentylmethyl)amino)methyl)pyridin-2-yl)-6-(3-((4-methyl-4H-1,2,4-triazol-3-yl)methyl)oxetan-3-yl)isoindolin-1-one (Compound 159);
(S)-2-(6-Chloro-4-(((cyclopropylmethyl)amino)methyl)pyridin-2-yl)-6-(1-(4-methyl-4H-1,2,4-triazol-3-yl)propan-2-yl)isoindolin-1-one (Compound 160);
(R)-2-(6-chloro-4-(((cyclopropylmethyl)amino)methyl)pyridin-2-yl)-6-(1-(4-methyl-4H-1,2,4-triazol-3-yl)propan-2-yl)isoindolin-1-one (Compound 161);
2-(4-(((Cyclobutylmethyl)amino)methyl)-6-(trifluoromethyl)pyridin-2-yl)-6-(3-((4-methyl-4H-1,2,4-triazol-3-yl)methyl)oxetan-3-yl)isoindolin-1-one (Compound 162);
(S)-2-(6-Chloro-4-(((3,3,3-trifluoropropyl)amino)methyl)pyridin-2-yl)-6-(1-(4-methyl-4H-1,2,4-triazol-3-yl)propan-2-yl)isoindolin-1-one (Compound 163);
(R)-2-(6-Chloro-4-(1-((cyclopropylmethyl)amino)ethyl)pyridin-2-yl)-6-(3-((4-methyl-4H-1,2,4-triazol-3-yl)methyl)oxetan-3-yl)isoindolin-1-one (Compound 166);
(S)-2-(6-Chloro-4-(1-((cyclopropylmethyl)amino)ethyl)pyridin-2-yl)-6-(3-((4-methyl-4H-1,2,4-triazol-3-yl)methyl)oxetan-3-yl)isoindolin-1-one (Compound 167);
3-(2-(6-Chloro-4-(((cyclopropylmethyl)amino)methyl)pyridin-2-yl)-3-oxoiso-indolin-5-yl)-3-((4-methyl-4H-1,2,4-triazol-3-yl)methyl)cyclobutane-1-carbonitrile (Compound 168);
2-(6-Chloro-4-((((3,3-difluorocyclobutyl)methyl)amino)methyl)pyridin-2-yl)-6-(3-((4-methyl-4H-1,2,4-triazol-3-yl)methyl)oxetan-3-yl)isoindolin-1-one (Compound 169);
(R)-2-(6-Chloro-4-(((cyclobutylmethyl)amino)methyl)pyridin-2-yl)-6-(1-(4-methyl-4H-1,2,4-triazol-3-yl)propan-2-yl)isoindolin-1-one (Compound 170);
(S)-2-(6-Chloro-4-(((cyclobutylmethyl)amino)methyl)pyridin-2-yl)-6-(1-(4-methyl-4H-1,2,4-triazol-3-yl)propan-2-yl)isoindolin-1-one (Compound 171);
2-(6-Chloro-4-((((1-methylcyclobutyl)methyl)amino)methyl)pyridin-2-yl)-6-(3-((4-methyl-4H-1,2,4-triazol-3-yl)methyl)oxetan-3-yl)isoindolin-1-one (Compound 172);
2-(6-Chloro-4-((((2,2-difluorocyclopropyl)methyl)amino)methyl)pyridin-2-yl)-6-(3-((4-methyl-4H-1,2,4-triazol-3-yl)methyl)oxetan-3-yl)isoindolin-1-one (Compound 175);
(1S,3r)-3-(2-(6-Chloro-4-(((S)-3-methylpiperidin-1-yl)methyl)pyridin-2-yl)-3-oxoisoindolin-5-yl)-3-((4-methyl-4H-1,2,4-triazol-3-yl)methyl)cyclobutane-1-carbo-nitrile (Compound 179);
2-(6-Chloro-4-(((S)-3-methylpiperidin-1-yl)methyl)pyridin-2-yl)-6-((S)-1,1,1-trifluoro-3-(4-methyl-4H-1,2,4-triazol-3-yl)propan-2-yl)isoindolin-1-one (Compound 181);
3-(2-(6-Chloro-4-(((cyclobutylmethyl)amino)methyl)pyridin-2-yl)-3-oxoiso-indolin-5-yl)-3-((4-methyl-4H-1,2,4-triazol-3-yl)methyl)cyclobutane-1-carbonitrile (Compound 182);
2-(6-Chloro-4-((((tetrahydrofuran-2-yl)methyl)amino)methyl)pyridin-2-yl)-6-(3-((4-methyl-4H-1,2,4-triazol-3-yl)methyl)oxetan-3-yl)isoindolin-1-one (Compound 188);
2-(6-Chloro-4-(((2-hydroxycyclopentyl)amino)methyl)pyridin-2-yl)-6-(3-((4-methyl-4H-1,2,4-triazol-3-yl)methyl)oxetan-3-yl)isoindolin-1-one (Compound 193);
(S)-2-(6-Chloro-4-((((tetrahydrofuran-2-yl)methyl)amino)methyl)pyridin-2-yl)-6-(3-((4-methyl-4H-1,2,4-triazol-3-yl)methyl)oxetan-3-yl)isoindolin-1-one (Compound 196);
2-(6-Chloro-4-(1-(((2,2-difluorocyclopropyl)methyl)amino)ethyl)pyridin-2-yl)-6-(3-((4-methyl-4H-1,2,4-triazol-3-yl)methyl)oxetan-3-yl)isoindolin-1-one (Compound 199);
2-(6-Chloro-4-((cyclopentylamino)methyl)pyridin-2-yl)-6-(3-((4-methyl-4H-1,2,4-triazol-3-yl)methyl)oxetan-3-yl)isoindolin-1-one (Compound 201);
2-(6-Chloro-4-(((3,3,3-trifluoro-2-methylpropyl)amino)methyl)pyridin-2-yl)-6-(3-((4-methyl-4H-1,2,4-triazol-3-yl)methyl)oxetan-3-yl)isoindolin-1-one (Compound 202);
2-(6-Chloro-4-(1-(((2,2-difluorocyclopropyl)methyl)amino)ethyl)pyridin-2-yl)-6-(3-((4-methyl-4H-1,2,4-triazol-3-yl)methyl)oxetan-3-yl)isoindolin-1-one (Compound 206);
2-(6-Chloro-4-(((2-hydroxycyclopentyl)amino)methyl)pyridin-2-yl)-6-(3-((4-methyl-4H-1,2,4-triazol-3-yl)methyl)oxetan-3-yl)isoindolin-1-one (Compound 209);
2-(6-Chloro-4-(1-((cyclobutylmethyl)amino)ethyl)pyridin-2-yl)-6-(3-((4-methyl-4H-1,2,4-triazol-3-yl)methyl)oxetan-3-yl)isoindolin-1-one (Compound 213);
2-(6-Chloro-4-((R)-1-((((S)-tetrahydrofuran-2-yl)methyl)amino)ethyl)pyridin-2-yl)-6-(3-((4-methyl-4H-1,2,4-triazol-3-yl)methyl)oxetan-3-yl)isoindolin-1-one (Compound 219);
(S)-2-(6-Chloro-4-(1-(((3,3-difluorocyclobutyl)methyl)amino)ethyl)pyridin-2-yl)-6-(3-((4-methyl-4H-1,2,4-triazol-3-yl)methyl)oxetan-3-yl)isoindolin-1-one (Compound 222);
(R)-2-(6-Chloro-4-(((1-cyclopropylethyl)amino)methyl)pyridin-2-yl)-6-(3-((4-methyl-4H-1,2,4-triazol-3-yl)methyl)oxetan-3-yl)isoindolin-1-one (Compound 223);
3-(2-(6-Chloro-4-(1-((cyclopropylmethyl)amino)ethyl)pyridin-2-yl)-3-oxoiso-indolin-5-yl)-3-((4-methyl-4H-1,2,4-triazol-3-yl)methyl)cyclobutane-1-carbonitrile (Compound 225);
3-(2-(6-Chloro-4-((((3,3-difluorocyclobutyl)methyl)amino)methyl)pyridin-2-yl)-3-oxoisoindolin-5-yl)-3-((4-methyl-4H-1,2,4-triazol-3-yl)methyl)cyclobutane-1-carbo-nitrile (Compound 227);
3-(2-(6-Chloro-4-((((2,2-difluorocyclopropyl)methyl)amino)methyl)pyridin-2-yl)-3-oxoisoindolin-5-yl)-3-((4-methyl-4H-1,2,4-triazol-3-yl)methyl)cyclobutane-1-carbonitrile (Compound 230);
(R)-2-(6-Chloro-4-(1-(cyclopentylamino)ethyl)pyridin-2-yl)-6-(3-((4-methyl-4H-1,2,4-triazol-3-yl)methyl)oxetan-3-yl)isoindolin-1-one (Compound 231);
(S)-2-(6-Chloro-4-(1-(cyclopentylamino)ethyl)pyridin-2-yl)-6-(3-((4-methyl-4H-1,2,4-triazol-3-yl)methyl)oxetan-3-yl)isoindolin-1-one (Compound 232);
(1r,3r)-3-(2-(6-Chloro-4-(((cyclopropylmethyl)amino)methyl)pyridin-2-yl)-3-oxoisoindolin-5-yl)-3-((4-methyl-4H-1,2,4-triazol-3-yl)methyl)cyclobutane-1-carbo-nitrile (Compound 233);
(1s,3s)-3-(2-(6-Chloro-4-(((cyclopropylmethyl)amino)methyl)pyridin-2-yl)-3-oxoisoindolin-5-yl)-3-((4-methyl-4H-1,2,4-triazol-3-yl)methyl)cyclobutane-1-carbonitrile (Compound 234);
2-(6-Chloro-4-((R)-1-(((S)-1-cyclopropylethyl)amino)ethyl)pyridin-2-yl)-6-(3-((4-methyl-4H-1,2,4-triazol-3-yl)methyl)oxetan-3-yl)isoindolin-1-one (Compound 239);
(1r,3r)-3-(2-(6-Chloro-4-(((cyclobutylmethyl)amino)methyl)pyridin-2-yl)-3-oxoisoindolin-5-yl)-3-((4-methyl-4H-1,2,4-triazol-3-yl)methyl)cyclobutane-1-carbonitrile (Compound 244);
2-(6-Chloro-4-(1-((2-cyclopropylpropan-2-yl)amino)ethyl)pyridin-2-yl)-6-(3-((4-methyl-4H-1,2,4-triazol-3-yl)methyl)oxetan-3-yl)isoindolin-1-one (Compound 247);
2-(6-Chloro-4-(1-(cyclopentylamino)propyl)pyridin-2-yl)-6-(3-((4-methyl-4H-1,2,4-triazol-3-yl)methyl)oxetan-3-yl)isoindolin-1-one (Compound 251);
2-(6-Chloro-4-(((2-methoxycyclopentyl)amino)methyl)pyridin-2-yl)-6-(3-((4-methyl-4H-1,2,4-triazol-3-yl)methyl)oxetan-3-yl)isoindolin-1-one (Compound 253);
2-(6-Chloro-4-(((cyclopropylmethyl)amino)methyl)pyridin-2-yl)-6-(2-((4-methyl-4H-1,2,4-triazol-3-yl)methyl)spiro[3.3]heptan-2-yl)isoindolin-1-one (Compound 255);
2-(4-(((Cyclopropylmethyl)amino)methyl)-6-methylpyridin-2-yl)-6-(4-fluoro-2-(4-methyl-4H-1,2,4-triazol-3-yl)phenyl)-4-(trifluoromethyl)isoindolin-1-one (Compound 256);
2-(4-((((3,3-Difluorocyclobutyl)methyl)amino)methyl)-6-methylpyridin-2-yl)-6-(4-fluoro-2-(4-methyl-4H-1,2,4-triazol-3-yl)phenyl)isoindolin-1-one (Compound 260);
2-(4-(((Cyclopropylmethyl)amino)methyl)-5-fluoro-6-methylpyridin-2-yl)-6-(4-fluoro-2-(4-methyl-4H-1,2,4-triazol-3-yl)phenyl)isoindolin-1-one (Compound 261);
6-(5-Chloro-3-(4-methyl-4H-1,2,4-triazol-3-yl)pyridin-2-yl)-2-(4-(((cyclo-propylmethyl)amino)methyl)-6-methylpyridin-2-yl)isoindolin-1-one (Compound 263);
2-(4-(((Cyclopropylmethyl)amino)methyl)-6-(trifluoromethyl)pyridin-2-yl)-6-(4-fluoro-2-(4-methyl-4H-1,2,4-triazol-3-yl)phenyl)isoindolin-1-one (Compound 265);
2-(4-(((Cyclopropylmethyl)amino)methyl)-6-methylpyridin-2-yl)-6-(4-methoxy-2-(4-methyl-4H-1,2,4-triazol-3-yl)phenyl)isoindolin-1-one (Compound 273);
6-(4-Chloro-2-(4-methyl-4H-1,2,4-triazol-3-yl)phenyl)-2-(4-(((cyclobutyl-methyl)amino)methyl)-6-methylpyridin-2-yl)isoindolin-1-one (Compound 274);
6-(4-Chloro-2-(4-methyl-4H-1,2,4-triazol-3-yl)phenyl)-2-(6-chloro-4-(2-((cyclopropylmethyl)amino)propan-2-yl)pyridin-2-yl)isoindolin-1-one (Compound 275);
2-(4-(((Cyclopropylmethyl)amino)methyl)-6-methylpyridin-2-yl)-6-(4-methyl-2-(4-methyl-4H-1,2,4-triazol-3-yl)phenyl)isoindolin-1-one (Compound 277);
6-(4-Chloro-2-(4-methyl-4H-1,2,4-triazol-3-yl)phenyl)-2-(4-(1-((cyclobutyl-methyl)amino)ethyl)-6-methylpyridin-2-yl)isoindolin-1-one (Compound 278);
6-(4-Chloro-2-(4-methyl-4H-1,2,4-triazol-3-yl)phenyl)-2-(4-((((3,3-difluoro-cyclobutyl)methyl)amino)methyl)-6-methylpyridin-2-yl)isoindolin-1-one (Compound 279);
6-(4-Chloro-2-(4-methyl-4H-1,2,4-triazol-3-yl)phenyl)-2-(3-(((cyclopropyl-methyl)amino)methyl)-5-methylphenyl)isoindolin-1-one (Compound 280);
6-(4-Chloro-2-(4-methyl-4H-1,2,4-triazol-3-yl)phenyl)-2-(4-(1-(((3,3-difluoro-cyclobutyl)methyl)amino)ethyl)-6-methylpyridin-2-yl)isoindolin-1-one (Compound 281);
2-(4-(1-((Cyclopropylmethyl)amino)ethyl)-6-methylpyridin-2-yl)-6-(4-fluoro-2-(4-methyl-4H-1,2,4-triazol-3-yl)phenyl)isoindolin-1-one (Compound 282);
2-(4-(((Cyclopropylmethyl)amino)methyl)-6-methylpyridin-2-yl)-6-(4-fluoro-2-(4-methylisoxazol-3-yl)phenyl)isoindolin-1-one (Compound 289);
2-(4-(2-((Cyclopropylmethyl)amino)propan-2-yl)-6-methylpyridin-2-yl)-6-(4-fluoro-2-(4-methyl-4H-1,2,4-triazol-3-yl)phenyl)isoindolin-1-one (Compound 293);
2-(4-(((Cyclopropylmethyl)amino)methyl)-6-methylpyridin-2-yl)-6-(2-(4-ethyl-4H-1,2,4-triazol-3-yl)-4-fluorophenyl)isoindolin-1-one (Compound 294);
2-(4-((sec-Butylamino)methyl)-6-methylpyridin-2-yl)-6-(4-fluoro-2-(4-methyl-4H-1,2,4-triazol-3-yl)phenyl)isoindolin-1-one (Compound 295);
2-(4-(((Dicyclopropylmethyl)amino)methyl)-6-methylpyridin-2-yl)-6-(4-fluoro-2-(4-methyl-4H-1,2,4-triazol-3-yl)phenyl)isoindolin-1-one (Compound 298);
6-(4-Fluoro-2-(4-methyl-4H-1,2,4-triazol-3-yl)phenyl)-2-(4-((isopentylamino)-methyl)-6-methylpyridin-2-yl)isoindolin-1-one (Compound 300);
6-(4-Fluoro-2-(4-methyl-4H-1,2,4-triazol-3-yl)phenyl)-2-(4-(((2-methoxyethyl)-amino)methyl)-6-methylpyridin-2-yl)isoindolin-1-one (Compound 302);
6-(4-Fluoro-2-(4-methyl-4H-1,2,4-triazol-3-yl)phenyl)-2-(6-methyl-4-((neo-pentylamino)methyl)pyridin-2-yl)isoindolin-1-one (Compound 303);
2-(4-(((2-Cyclopropylethyl)amino)methyl)-6-methylpyridin-2-yl)-6-(4-fluoro-2-(4-methyl-4H-1,2,4-triazol-3-yl)phenyl)isoindolin-1-one (Compound 307);
6-(4-Fluoro-2-(4-methyl-4H-1,2,4-triazol-3-yl)phenyl)-2-(4-(((2-hydroxy-2-methylpropyl)amino)methyl)-6-methylpyridin-2-yl)isoindolin-1-one (Compound 309);
2-(4-(((Cyclopropylmethyl)amino)methyl)-6-methylpyridin-2-yl)-6-(4-fluoro-2-(4-(methyl-d3)-4H-1,2,4-triazol-3-yl)phenyl)isoindolin-1-one (Compound 310);
6-(4-Fluoro-2-(4-methyl-4H-1,2,4-triazol-3-yl)phenyl)-2-(4-((((1R,2R)-2-hydroxycyclopentyl)amino)methyl)-6-methylpyridin-2-yl)isoindolin-1-one (Compound 314);
(R)-6-(4-Fluoro-2-(4-methyl-4H-1,2,4-triazol-3-yl)phenyl)-2-(4-(((1-hydroxy-3-methylbutan-2-yl)amino)methyl)-6-methylpyridin-2-yl)isoindolin-1-one (Compound 315);
6-(4-Fluoro-2-(4-methyl-4H-1,2,4-triazol-3-yl)phenyl)-2-(4-((((1-hydroxy-cyclopentyl)methyl)amino)methyl)-6-methylpyridin-2-yl)isoindolin-1-one (Compound 319);
6-(2-(4-Cyclopropyl-4H-1,2,4-triazol-3-yl)-4-fluorophenyl)-2-(4-(((cyclopropyl-methyl)amino)methyl)-6-methylpyridin-2-yl)isoindolin-1-one (Compound 320);
6-(4-Fluoro-2-(4-methyl-4H-1,2,4-triazol-3-yl)phenyl)-2-(6-methyl-4-((propyl-amino)methyl)pyridin-2-yl)isoindolin-1-one (Compound 323);
6-(4-Fluoro-2-(4-methyl-4H-1,2,4-triazol-3-yl)phenyl)-2-(6-methyl-4-(((oxetan-2-ylmethyl)amino)methyl)pyridin-2-yl)isoindolin-1-one (Compound 325);
6-(4-Fluoro-2-(4-methyl-4H-1,2,4-triazol-3-yl)phenyl)-2-(6-methyl-4-(((3,3,3-trifluoropropyl)amino)methyl)pyridin-2-yl)isoindolin-1-one (Compound 326);
6-(4-Fluoro-2-(4-methyl-4H-1,2,4-triazol-3-yl)phenyl)-2-(6-methyl-4-(((4,4,4-trifluorobutyl)amino)methyl)pyridin-2-yl)isoindolin-1-one (Compound 328);
2-(4-(((Cyclopentylmethyl)amino)methyl)-6-methylpyridin-2-yl)-6-(4-fluoro-2-(4-methyl-4H-1,2,4-triazol-3-yl)phenyl)isoindolin-1-one (Compound 329);
2-(4-(((Cyclopropylmethyl)amino)methyl)-6-methyl-5-(trifluoromethyl)pyridin-2-yl)-6-(4-fluoro-2-(4-methyl-4H-1,2,4-triazol-3-yl)phenyl)isoindolin-1-one (Compound 337);
or a tautomer or a pharmaceutically acceptable salt thereof.

38. A compound according to claim 1 for use as a medicament.

39. A compound for use according to claim 38, for use in the treatment of a disease or condition wherein inhibition of Cbl-b is desired.

40. A compound for use according to claim 39, wherein the disease is cancer.

41. A compound for use according to claim 40, wherein the cancer is squamous cell carcinoma, bladder cancer, gastric cancer, liver cancer, lung cancer and colon cancer, bone cancer, pancreatic cancer, melanoma, uterine cancer, ovarian cancer, rectal cancer, stomach cancer, testicular cancer, uterine cancer, endometrial cancer, non-Hodgkin's lymphoma or leukemia.

42. A compound for use according to any one of claims to 38-41, wherein a therapeutically effective amount of a compound of formula (I) or a pharmaceutically acceptable salt thereof is administered in addition to one or more anti-cancer agents.

43. A method for the treatment of a disease or condition wherein inhibition of Cbl-b is desired comprising administering to a subject in need thereof a therapeutically effective amount of a compound according to claim 1.

44. A method according to claim 43 wherein the disease is cancer.

45. A method according to claim 44 wherein the cancer is squamous cell carcinoma, bladder cancer, gastric cancer, liver cancer, lung cancer and colon cancer, bone cancer, pancreatic cancer, melanoma, uterine cancer, ovarian cancer, rectal cancer, stomach cancer, testicular cancer, uterine cancer, endometrial cancer, non-Hodgkin's lymphoma or leukemia.

46. A method according to any of claims 43 to 45, wherein a therapeutically effective amount of a compound of formula (I) or a pharmaceutically acceptable salt thereof is administered in addition to one or more anti-cancer agents.

47. A pharmaceutical composition comprising a compound according to any of claims 1 to 37 together with a pharmaceutically acceptable carrier.
